(19) United States
(12) Patent Application Publication
(10) Pub. No.: US 2015/0133469 A1
(43) Pub. Date: May 14, 2015

(54) EARLY DETECTION OF TUBERCULOSIS TREATMENT RESPONSE

(71) Applicants: BAYLOR RESEARCH INSTITUTE, Dallas, TX (US); MEDICAL RESEARCH COUNCIL, Swindon (GB); IMPERIAL COLLEGE HEATHCARE NHS TRUST, London (GB)

(72) Inventors: Anne O'Garra, London (GB); Chloe Bloom, London (GB); Matthew Paul Reddoch Berry, London (GB); Robert Wilkinson, Observatory (ZA); Jacques F. Banchereau, Montclair, NJ (US); Damien Chaussabel, Bainbridge Island, WA (US); Maria Virginia Pascual, Dallas, TX (US)

(21) Appl. No.: 14/384,891

(22) PCT Filed: Mar. 13, 2013
(86) PCT No.: PCT/US2013/030986

§ 371 Date: Sep. 12, 2014

Related U.S. Application Data
(60) Provisional application No. 61/610,121, filed on Mar. 13, 2012.

(51) Int. Cl. C12Q 1/68; A61K 31/133; A61K 31/455; A61K 31/496; A61K 31/4965

(52) U.S. Cl. CPC C12Q 1/6883; A61K 31/496; A61K 31/4965; A61K 31/455; A61K 31/133; C12Q 2600/158; C12Q 2600/118
USPC 514/254.11

(57) ABSTRACT

The present invention includes methods for early detection of a treatment response in a patient suspected of being infected with Mycobacterium tuberculosis. Changes in the blood transcriptome are detectable within two weeks of the initiation of antituberculosis therapy.

### CROSS-REFERENCE TO RELATED APPLICATIONS

This application claims priority to, and incorporates by reference in its entirety, provisional patent application Ser. No. 61/610,121 filed Mar. 13, 2012, and which is also titled “Early Detection of Tuberculosis Treatment Response.”

### TECHNICAL FIELD OF THE INVENTION

The present invention relates in general to the field of Mycobacterium tuberculosis infection, and more particularly, to methods for monitoring treatment response and determining treatment effectiveness.

### STATEMENT OF FEDERALLY FUNDED RESEARCH

None.

### BACKGROUND OF THE INVENTION

Without limiting the scope of the invention, its background is described in connection with the treatment of Mycobacterium tuberculosis infection.

United States Patent Application Publication No. 2009/0104602, entitled “Diagnosis of Tuberculosis,” filed by Fernandez-Reyes et al. describes methods of diagnosing tuberculosis comprising: (i) providing expression data of two or more markers in a subject, wherein at least two of said markers are selected from transthyretin, neopterin, C-reactive protein (CRP), serum amyloid A (SAA), serum albumin, apoliopoprotein-A1 (Apo-A1), apolipoprotein-A2 (Apo-A2), hemoglobin beta, haptoglobin protein, DEP domain protein, leucine-rich alpha-2-glycoprotein (A2GL) and hypothetical protein DFKZp6671032; and (ii) comparing said expression data to expression data of said marker from a group of control subjects, wherein said control subjects comprise patients suffering from inflammatory conditions other than tuberculosis (TB), thereby determining whether or not said test subject has TB.

United States Patent Application Publication No. 2003/0138813, entitled “Method of diagnosis and disease risk assessment,” filed by Engstrand et al., relates to methods of determining information about the likely clinical outcome of a microbiological infection in a patient and also to methods of selecting a suitable therapeutic regimen for a patient with a microbiological infection. The application describes analyzing the virulence gene of Mycobacterium tuberculosis to determine the likely clinical outcome.

Nahid et al., CDC/NIH Workshop Report, “Tuberculosis biomarker and surrogate endpoint research roadmap,” Am J Respir Crit Care Med. 2011 Oct. 15; 184(8):972-9, states that Centers for Disease Control and Prevention and National Institutes of Health convened a multidisciplinary meeting to discuss surrogate markers of treatment response in tuberculosis. It is said that, at a minimum, a biomarker of treatment response most useful for drug development would need to: 1) correspond closely with treatment outcomes; 2) have a wide dynamic range that would allow analysis as a continuous variable; and 3) provide this information from a limited number of early time points.

Wallis et al., “Biomarkers and diagnostics for tuberculosis: progress, needs, and translation into practice,” Lancet 2010 May 29; 375(9729):1920-37. Epub 2010 May 18, states that host or pathogen-specific tuberculosis biomarkers provide prognostic information, either for individual patients or study cohorts. It is said that detection of volatile organic compounds in the breath of patients with pulmonary tuberculosis has been reported, but no study has reported changes during treatment. It is stated that studies have examined levels of M. tuberculosis antigen 85 and 85B RNA in sputum during treatment, and the magnitude and duration of increases in this protein during the first week of treatment predicted relapse or failure in four of 42 patients.

Berry et al., “An interferon-inducible neutrophil-driven blood transcriptional signature in human tuberculosis,” Nature 2010 Aug. 19; 466(7309):973-7, reports identification of a whole-blood 393 transcript signature for active tuberculosis in intermediate and high-burden settings, which is missing in the majority of individuals with latent tuberculosis, and missing from healthy controls. The signature correlated with radiological extent of disease and diminished after two months of treatment and reverted to that of healthy controls after completion of treatment. An 86-transcript signature discriminated between active TB and other inflammatory and infectious diseases, and this signature was also diminished after two months of treatment. The tuberculosis signature was dominated by a neutrophil-driven interferon (IFN)-inducible gene profile, consisting of both IFN-gamma and type I IFN-alpha beta signaling.

Marchant et al., “Serological markers of disease activity in tuberculosis and HIV infection,” Clin Exp Immunol. 2000 October; 122(1):10-2, states that markers of disease activity are needed to evaluate disease progression and to monitor response to therapy. It is suggested that e.g., soluble tumour necrosis factor receptor type 1 (sTNF-RI) and beta 2-macroglobulin, could be used as independent markers of disease activity in TB and HIV infection, respectively.

Frahm et al., “Discriminating between latent and active tuberculosis with multiple biomarker responses,” Tuberculosis (Edinb). 2011 May; 91(3):250-6. Epub 2011 Mar. 10, states that twenty-five biomarkers were evaluated and it was found that IL-15 and MCP-1 identified 83% of active and 88% of latent infections. The combination of IL-15 and MCP-1 responses was accurate in distinguishing persons with active tuberculosis from persons with latent tuberculosis in this study.

### SUMMARY OF THE INVENTION

Part of the inventive subject matter that the present invention provides includes methods for early detection of a treatment response in a patient suspected of being infected with Mycobacterium tuberculosis. In some embodiments, changes in the blood transcriptome are detectable within two weeks or less of the initiation of antituberculosis therapy.

In one aspect, a method is provided for evaluating tuberculosis treatment response in a patient, the method comprising: measuring expression levels of genes in a biological sample from the patient to generate a first gene expression profile, wherein the biological sample is obtained at a first time point that is before or concurrent with commencement of tuberculosis treatment; measuring expression levels of genes in a second biological sample from the patient to generate a second gene expression profile, wherein the second biological sample is obtained after commencement of the treatment but at two weeks or less after commencement; and calculating a temporal molecular response value by comparing the first and second gene expression profiles. In a related aspect, a method is provided for evaluating effectiveness of tuberculosis treatment, the method comprising: measuring expression levels of genes in a biological sample from a tuberculosis patient to generate a first gene expression profile, wherein the biological sample is obtained at a first time point that is before or concurrent with commencement of tuberculosis treatment; administering the treatment to the patient; measuring expression levels of genes in a second biological sample from the patient to generate a second gene expression profile, wherein the second biological sample is obtained after commencement of the treatment but at two weeks or less after commencement; and calculating a temporal molecular response value by comparing the first and second gene expression profiles. In some embodiments, a significant temporal molecular response value is a biomarker for an effective treatment.

In a further related aspect, a method is provided for treating a patient with Mycobacterium tuberculosis infection, the method comprising: measuring expression levels of genes in a biological sample from the patient to generate a first gene expression profile, wherein the biological sample is obtained at a first time point that is before or concurrent with commencement of treatment for the infection; administering a treatment for the infection to the patient; measuring expression levels of genes in a second biological sample from the patient to generate a second gene expression profile, wherein the second biological sample is obtained after commencement of the treatment but at two weeks or less after commencement; and calculating a temporal molecular response value by comparing the first and second gene expression profiles. In an associated method, the treatment is continued if the temporal molecular response value is significant. In a further associated method, the treatment is discontinued if the temporal molecular response value is not significant.

In some embodiments, the biological sample is blood. In addition, a gene expression profile may comprise RNA transcriptome expression data. Genes of a gene expression profile may comprise 2, 3, 4, 5, 6, 7, 8, 9, or 10 genes, between 11 and 20 genes, between 21 and 30 genes, between 31 and 50 genes, between 51 and 75 genes, between 76 and 100 genes, between 101 and 200 genes, between 201 and 300 genes, between 301 and 500 genes, between 501 and 750 genes, or more than 751 genes. In addition, genes of a gene expression profile may comprise genes selected from Table 1, 3, 4, 5, 6, 7, 8, 9, 10, 11, or 12 disclosed herein.

In related embodiments of these methods, the second time point is 13, 12, 11, 10, 9, 8, 7, 6, 5, 4, 3, or 2 days or less, or 1 day or less, after commencement of treatment. In other related embodiments, genes of the gene expression profile comprise 1, 2, 3, 4, 5, 6 or more different genes selected from the group consisting of IF135, IFIT1, IFIT3, IFITM1, IRF1, JAK2, SOCS1, STAT1, TAP1, CD40LG, CD79A, CD79B, FAS, FCER1G, IL15, IL1B, IL1RN, SLAMF1, TLR2, TLR5, TNFSF13B, C2, C1QB, C1QC, C4BPA, CD59, CR1, SERPING1, C5, CASP1, IFIH1, IL1B, IRF7, NLRC4, NOD2, MAPK14, OSM, SOCS3, CD86, CXCL10, FCER1G, TLR8, CD86, CREB5, FCGR1A, FCGR1B, IL15, IL23A, STAT2, CASP5, ITGAX, PLCG1, F2RL1, IL18R1, IL18RAP, IRAK3, NFAT5, PDGFA, PLCG1, TRAF5, CD3E, FCGR1C, FCGR2C, FCGR3B, and LCK.

In one embodiment, the present invention includes a method to determine effectiveness of a treatment for tuberculosis in a patient suspected of being infected with Mycobacterium tuberculosis, the method comprising: obtaining a first gene expression dataset from a sample of the patient at a first time point, wherein the first time point is before or simultaneous with a commencement of the treatment for tuberculosis; obtaining a second gene expression dataset from the sample of the patient at a second time point, wherein the second time point is less than 2 months after the commencement of the treatment for tuberculosis, wherein the first gene expression dataset and the second gene expression dataset comprises one or more genes; comparing the first gene expression dataset with the second gene expression dataset; determining that treatment is effective if a significant change between the first gene expression dataset and the second gene expression dataset is detected; or determining that treatment is ineffective if no change or less than a significant change between the first gene expression dataset and the second gene expression dataset is detected. In one aspect, the gene expression data set comprises 2, 3, 4, 5, 6, 7, 8, 9, or 10 genes, between 10 and 19 genes, between 20 and 99 genes or 100 or more genes of genes listed in table 1, 3, 4, 5, 6, 7, 8, 9, 10, 11, or 12. In another aspect, the gene expression data set comprises one or more genes selected from the group consisting of genes related to IFN Signaling selected from the group consisting of IF135, IFIT1, IFIT3, IFITM1, OAS1, IRF1, JAK2, SOCS1, STAT1, STAT2, and TAP1, significantly changed upon two weeks after initiation of anti-TB drug treatment. In another aspect, the gene expression data set comprises one or more genes selected from the group consisting of genes related to T and B cell signaling selected from the group consisting of CD40LG, CD79A, CD79A, CD79B, FAS, FCER1G, IL15, IL23A, IL1B, IL1RN, SLAMF1, TLR2, TLR5, TLR8, TNFSF13B, TNFRSF13B, and CD86. In another aspect, the gene expression data set comprises one or more genes selected from the group consisting of genes related to a complement system selected from the group consisting of C2, C1QB, C1QC, C4BPA, CD59, CR1, and SERPING1. In another aspect, the gene expression data set comprises one or more genes selected from the group consisting of genes having a role in pattern recognition selected from the group consisting of C5, C1QB, C1QC, CASP1, IFIH1, IL1B, IRF7, NLRC4, OAS1, OAS2, OAS3, NOD2, TLR2, TLR5, TLR8, and C3AR1. In another aspect, the gene expression data set comprises one or more genes selected from the group consisting of genes related to JAK family kinases in IL-6 type cytokine signaling selected from the group consisting of MAPK14, OSM, SOCS1, SOCS3, and STAT1. In another aspect, the gene expression data set comprises one or more genes selected from the group consisting of genes having a role in communication between innate and adaptive immune cells selected from the group consisting of CD86, CD40LG, CXCL10, FCER1G, IL15, IL1B, IL1RN, TLR2, TLR5, TLR8, TNFRSF13B, and TNFSF13B. In another aspect, the gene expression data set comprises one or more genes selected from the group consisting of genes related to dendritic cell maturation selected from the group consisting of CD86, CD40LG, CREB5, FCER1G, FCGR1A, FCGR1B, IL15, IL1B, IL1RN, IL23A, JAK2, MAPK14, STAT1, STAT2, and TLR2. In another aspect, the gene expression data set comprises one or more genes selected from the group consisting of genes related to TREM signaling selected from the group consisting of CASP1, CASP5, IL1B, ITGAX, JAK2, NOD2, PLCG1, TLR2, and TLR5. In another aspect, the gene expression data set comprises one or more genes selected from the group consisting of genes related to a role of macrophages, fibroblasts, and endothelial cells in rheumatoid arthritis selected from the group consisting of C5, CREB5, F2RL1, FCGR1A, IL15, IL18R1, IL18RAP, IL1B, IL1RN, IRAK3, JAK2, MAPK14, NFAT5, OSM, PDGFA, PLCG1, SOCS1, SOCS3, TLR2, TLR5, TNFSF13B, and TRAF5. In another aspect, the gene expression data set comprises one or more genes selected from the group consisting of genes related to systemic lupus erythematous signaling selected from the group consisting of C5, CD3E, CD40LG, CD79A, CD79B, FCER1G, FCGR1A, FCGR1B, FCGR1C, FCGR2C, FCGR3B, IL1B, IL1RN, LCK, NFAT5, PLCG1, and TNFSF13B. In another aspect, the gene expression data set comprises one or more genes selected from the group consisting of IFI35, IFIT1, IFIT3, IFITM1, OAS1, IRF1, JAK2, SOCS1, STAT1, STAT2, TAP1, CD40LG, CD79A, CD79A, CD79B, FAS, FCER1G, IL15, IL23A, IL1B, IL1RN, SLAMF1, TLR2, TLR5, TLR8, TNFSF13B, TNFRSF13B, CD86, C2, C1QB, C1QC, C4BPA, CD59, CR1, SERPING1, C5, CASP1, IFIH1, IRF7, NLRC4, OAS2, OAS3, NOD2, TLR2, TLR5, TLR8, C3AR1, MAPK14, OSM, SOCS3, STAT1, CD86, CD40LG, CXCL10, FCER1G, IL15, IL1RN, TLR2, TLR5, TLR8, TNFRSF13B, TNFSF13B, CD86, CD40LG, CREB5, FCER1G, FCGR1A, FCGR1B, IL15, IL1RN, IL23A, JAK2, MAPK14, CASP5, ITGAX, JAK2, NOD2, PLCG1, TLR2, TLR5, CREB5, F2RL1, FCGR1A, IL15, IL18R1, IL18RAP, IL1RN, IRAK3, JAK2, MAPK14, NFAT5, OSM, PDGFA, PLCG1, SOCS3, TLR2, TLR5, TNFSF13B, TRAF5, CD3E, CD40LG, CD79A, FCER1G, FCGR1A, FCGR1B, FCGR1C, FCGR2C, FCGR3B, IL1RN, LCK, NFAT5, PLCG1, TNFSF13B.

In another aspect, the second time point is between the start of treatment and two weeks after commencement of treatment. In another aspect, the significant change between the first gene expression dataset and the second gene expression dataset comprises the sum of transcripts that are greater than 2-fold different between the first and second time points, expressed as a percentage of the total number of transcripts in each of the gene signatures (Temporal Molecular Response Algorithm derived for this study). In another aspect the second time point is between 2 weeks and 2 months, showing a significant change after the commencement of treatment. In another aspect, 2 months and 6 months after the commencement of treatment. In another aspect, the change between the first gene expression dataset and the second gene expression dataset comprises is at least twofold change of expression most significant as described by the Temporal Molecular Response from initiation of treatment to after 2 weeks. In another aspect, the change between the first gene expression dataset and the second gene expression is observed in between 10 and 100 percent of genes. In another aspect, the treatment comprises treatment with rifampin, pyrazinamide, isoniazid ethambutol, or combinations thereof. In certain aspects, the treatment comprises treatment with anti-mycobacterial drugs against drug-sensitive Mtb, including the addition or substitution of other anti-mycobacterial agents such as levofloxacin, moxifloxacin, prothioniamide, ethionamide, cycloserine, amikacin, streptomycin, kanamycin, para-amino salicylic acid, capreomycin, linezolid, TMC-205, or other similar drugs. In addition it could be applied to the monitoring of new drugs being tested for greater efficacy, and also new drugs tested against MDR- and XDR-TB.

Another embodiment is a method of performing a clinical trial to evaluate the effectiveness of a candidate drug believed to be useful in treating Mycobacterium tuberculosis, the method comprising: (a) obtaining a biological sample from a patient with a Mycobacterium tuberculosis infection; (b) from the patient sample determining a first gene expression dataset from the sample of the patient at a first time point, wherein the first time point is before or simultaneous with a commencement of the treatment for Mycobacterium tuberculosis in one or more biological sample of the patient; (b) administering a candidate drug to the patient, and obtaining a second gene expression dataset from a second sample obtained from the patient at a second time point, wherein the second time point is less than 2 months after commencement of the treatment for Mycobacterium tuberculosis, wherein the first gene expression dataset and the second gene expression comprises one or more genes; comparing the first gene expression dataset with the second gene expression dataset following the treatment with the candidate drug; and determining that treatment is effective if a significant change between the first gene expression dataset and the second gene expression dataset is detected or determining that treatment is ineffective if less than a significant change between the first gene expression dataset and the second gene expression dataset is detected.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a more complete understanding of the features and advantages of the present invention, reference is now made to the detailed description of the invention along with the accompanying figures and in which: FIGS. 1A through FIG. 10E are further detailed in Example 1 herein; FIGS. 11A through 18C are further detailed in Example 2 herein; and FIGS. 19 through 21 are detailed in Example 3 herein.

FIGS. 1A-1B illustrate numbers enrolled, assigned to cohorts, and included in the analysis of 2011 cohorts. As shown in FIG. 1A, a total of 67 active (29) and latent (38) TB patients were enrolled into an Untreated South Africa 2011 Cohort. A total of 20 active TB patients were included in an Treated South Africa 2011 Cohort. Eleven were randomised into an Active TB Training Set and nine into an Active TB Test Set. As shown in FIG. 1B, a total of eight active TB patients were enrolled into a Treated UK 2011 Cohort.

FIGS. 2A-2F illustrate that a blood gene expression treatment response is readily detectable after only two weeks of treatment and independent of the pre-treatment signature. For FIG. 2A, a profile plot of all detectable transcripts (16835) obtained without any filtering, in the treated active TB patients in the South Africa 2011 cohort, including patients with missing time points, is presented. It can be seen that gene expression changes after just two weeks of treatment. For FIG. 2B, 664 differentially expressed transcripts between untreated active and latent TB patients in the Untreated South Africa 2011 cohort were obtained by twofold change from the median and stringent statistical filtering (Mann Whitney, Bonferroni p<0.01). The heatmap shows dynamic change of gene expression in response to treatment in the Treated South Africa 2011 cohort normalized to the median of all transcripts. FIG. 2C illustrates Ingenuity Pathway Analysis (IPA) of the 664 transcripts and shows the top significant pathways. FIG. 2D illustrates Interferon signaling pathway from the 664 list in IPA. FIG. 2E illustrates that weighted molecular distance to health (MDTH) of the Treated South Africa 2011 cohort significantly diminishes over treatment time (Friedman, Dunns, bars represent median & IQR, ***=p<0.001, **=p<0.01, *=p<0.05). FIG. 2F documents that temporal molecular response further shows significant and early changes in response to anti-TB treatment (ANOVA repeated measures, Tukeys, bars represent mean & SD).

FIGS. 3A-3F illustrate that a specific treatment response signature significantly diminishes at two weeks and two months after initiation of treatment and after completion of treatment. A specific TB treatment response signature was derived from significantly differentially expressed genes between untreated samples in the South Africa Active TB Training Set and their corresponding six month samples using 391 transcripts. FIG. 3A shows a heatmap of South Africa 2011 Active TB Training Set, normalised to the median of all transcripts, shows transcripts differentiating over time in response to treatment. FIG. 3B illustrates that a temporal molecular response further shows significant and early changes in response to TB treatment in the Active TB Training Set (Repeated measures, Tukeys, bars represent mean & SD, ***=p<0.001, **=p<0.01, *=p<0.05). FIG. 3C displays a heatmap of South Africa 2011 Active TB Test Set, normalized to the median of all transcripts, and shows transcripts differentiating over time in response to treatment. FIG. 3D illustrates that a temporal molecular response also shows in the Active TB Test Set significant and early changes, significantly after two weeks of initiation of treatment, in response to TB treatment. The present inventors have developed this Temporal Molecular Response Algorithm for quantifying an active TB patient's individual response to treatment; it facilitates, enables, and is of advantage for use in the clinical setting and in drug development clinical trials. FIG. 3E shows the IPA of the 391 transcripts showing the most significant pathways. FIG. 3F illustrates a Venn diagram that shows many overlapping genes between the active TB 664-transcript signature and the treatment-specific 391-transcript signature.

FIGS. 4A-4B illustrate that each individual patient's transcriptional response (391 gene list) occurred at a variable rate for the 391 gene list, which represents differentially expressed genes derived from comparing the untreated expression profiles and their corresponding end-of-treatment (six months) expression profiles in the South Africa 2011 Active TB Training Set. FIG. 4A displays a heatmap of South Africa 2011 cohort Active TB Training Set, normalized to the median of all transcripts, and shows hierarchical clustered transcripts differentiating over time per individual. FIG. 4B illustrates that each patient's temporal molecular response diminishes in the Active TB Training Set cohort but at different rates.

FIGS. 5A-5C illustrate that change in treatment specific signature (391 gene list) is validated in an independent UK cohort. The 391 gene list is derived from the differentially expressed genes between the untreated and six month treated samples in the Treated South Africa 2011 cohort. FIG. 5A displays a heatmap of the Treated UK 2011 Cohort, normalized to the median of all transcripts, and shows diminution of the treatment specific transcriptional signature in the UK cohort in response to successful anti-TB treatment. In FIG. 5B, a temporal molecular response shows significant changes in response at two weeks in the UK cohort (linear mixed models, bars represent mean & SD, ***=p<0.001, **=p<0.01, *=p<0.05). FIG. 5C illustrates that a diminished response can be seen in each patient by his or her temporal molecular response with apparent different patient response rates.

FIG. 6 illustrates that the changing transcriptional response is independent of the magnitude of the untreated transcriptional response. Weighted molecular distance to health (MDTH) during treatment has been shown to correlate with radiological extent of active TB disease (Berry et al., Nature 2010; 466:973-977). FIG. 6 shows that MDTH of the 664-transcript signature does not significantly correlate with the temporal molecular treatment response at two weeks or two months compared to pre-treatment (Pearson's correlation, p<0.05), but does at six months and 12 months (Pearson's correlation, p<0.05). While the treatment response in FIG. 5 correlates with cure by MDTH and Temporal Molecular Response, the treatment response rate cannot be predicted by the magnitude of the transcriptional response as measured by the MDTH before treatment.

FIGS. 7A-7B illustrate that individual patient's transcriptional responses occurred at a variable rate in an independently validated test set—the 391 gene list, differentially expressed genes derived from comparing the untreated expression profiles and their corresponding end-of-treatment (six months) expression profiles in the South Africa 2011 Active TB Training Set. FIG. 7A displays a heatmap of South Africa 2011 cohort Active TB Test Set and shows hierarchical clustered transcripts normalised to the median of all transcripts, differentiating over time per individual. FIG. 7B illustrates each patient's temporal molecular response in the South Africa 2011 cohort Active TB Test Set.

FIGS. 8A-8C show that the Berry et al. (2010) 393-transcript and 86-transcript TB signatures significantly diminish in response to successful treatment in the South Africa 2011 Cohort. The 393-transcript and 86-transcript signatures were defined as described (Berry et al., Nature 2010; 466:973-977) as differentiating active TB patients from latent TB patients/healthy controls (393 signature), and differentiating active TB patients from patients with other inflammatory and infectious diseases (86 signature). Both signatures diminished in response to anti-TB treatment in the Treated South Africa 2011 cohort. FIG. 8A displays a heatmap that shows hierarchical clustering of the transcripts, normalized to the median of all transcripts, with samples grouped into time points. FIG. 8B displays a heatmap that shows hierarchical clustering of the transcripts, normalized to the median of all transcripts, with samples grouped per individual. FIG. 8C illustrates that temporal molecular response further shows significant and early changes in response to anti-TB treatment as early as two weeks after treatment initiation (ANOVA repeated measures, Tukeys, bars represent mean & SD, ***=p<0.001, **=p<0.01, *=p<0.05).

FIG. 9 illustrates the numbers enrolled, assigned to cohorts, and included in the analysis of a South Africa 2009 cohort. A total of 51 active and latent TB patients were enrolled into the South Africa 2009 Berry et al cohort (Nature 2010; 466:973-977). Forty-four of these patients were included in the Untreated South Africa 2011 cohort, where they were additionally sampled and monitored post-treatment.

FIGS. 10A-10E illustrate that a change in active TB transcriptional signatures derived by identical analysis from the different cohorts is still observed and is significant after two weeks. The active TB transcriptional signatures were shown for each cohort as unsupervised hierarchical clustering between the untreated active and latent TB samples, then by Ingenuity Pathway Analysis (IPA), then by forced grouping of the samples showing diminishing of the transcriptional signature in response to treatment in a Treated South Africa 2011 Cohort and lastly by the temporal molecular response. 2011 cohorts were processed on different Illumina HT12 BeadChip versions: V3 and V4. To compensate for this, the V3 probes were translated into V4 format; there are slightly fewer probes in V4 than V3. Transcripts were obtained by the same approach and unsupervised clustering showed distinct clustering of the active and latent TB samples in all three of the 2009 cohorts. IPA of the transcripts shows the most highly significant pathways contains IFN-signaling in all three cohorts. FIG. 10A shows that for UK training set 2009, 565 transcripts in Illumina HT-12 V3 BeadChip, translates to 540 transcripts in Illumina HT-12 V4. FIG. 10B shows that for UK test set 2009, 224 transcripts in Illumina HT-12 V3 BeadChip, translates to 214 transcripts in Illumina HT-12 V4 BeadChip. FIG. 10C shows that for South Africa cohort 2009, 711 transcripts in Illumina FIT-12 V3 BeadChip, translates to 684 transcripts in Illumina HT-12 V4 BeadChip. FIG. 10D displays a Venn diagram comparing the active TB transcriptional signatures from each 2009 cohort. FIG. 10E displays a Venn diagram comparing: 1) all overlapping transcripts in ≧2 segments of the Venn diagram in FIG. 10D (344 transcripts in Illumina HT-12 V3 BeadChip, translates to 332 transcripts in Illumina HT-12 V4); 2) the South Africa 2011 active TB 664-transcript signature; and 3) the South Africa 2011 treatment specific 391-transcript signature. Regardless of how this host blood transcriptional signature was derived it was significantly changed after two weeks post initiation of successful drug treatment.

FIGS. 11A-11B. South Africa: As illustrated in FIG. 11A, a total of 67 active and latent TB patients were enrolled into an untreated South Africa 2011 Cohort. A total of 29 active TB patients were included in a treated South Africa 2011 Cohort. Fifteen were randomised into an Active TB Training Set and fourteen into an Active TB Test Set. UK: As illustrated in FIG. 11B, a total of eight active TB patients were enrolled into the treated UK 2011 Cohort. See also FIG. 1 at doi:10.1371/journal.pone.0046191.g001 (Bloom et al. 2012 “Detectable changes in the blood transcriptome are present after two weeks of antituberculosis therapy,” PLOS ONE 7(10): e46191).

FIGS. 12A-12C. Active TB signatures of Berry et al (2010) also significantly diminish in response to successful treatment. 393-transcript and 86-transcript signatures were defined as described (Reis-Filho and Pusztai 2011; Lancet 378: 1812-1823) differentiating active TB patients from latent TB patients/healthy controls (393 signature), and differentiating active TB patients from patients with other inflammatory and infectious diseases (86 signature). Both signatures diminished in response to anti-TB treatment in the treated South Africa 2011 cohort. Heatmap of FIG. 12A displays hierarchical clustering of the transcripts, normalised to the median of all transcripts, with samples grouped into time points. Heatmap of FIG. 12B displays hierarchical clustering of the transcripts, normalised to the median of all transcripts, with samples grouped per individual. In FIG. 12C, temporal molecular response further shows significant and early changes in response to anti-TB treatment (linear mixed models, bars represent mean & 95% confidence intervals, ***p<0.001, **=p<0.01, *=p<0.05). Summary of demographics and clinical data include, as in FIG. 11A, for a South Africa 2011 cohort: Of the 29 untreated active TB patients, 16 were also included in the previous Berry et al (2010) study, and, of the 38 untreated latent TB patients, 17 were also included in the previous Berry et al. (2010) study. For the study results of FIG. 12A-12C, all untreated samples were processed again alongside all the other samples. The UK 2011 cohort is as described in FIG. 11B. See also Supporting FIG. 3 at doi:10.1371/journal.pone.0046191.g001 (Bloom et al. 2012).

FIGS. 13A-13E. A blood transcriptional response is detectable after two weeks of treatment. In FIG. 13A, a profile plot of a set of all detectable transcripts (15837), obtained without any filtering, in the treated active TB patients in the South Africa 2011 cohort is displayed. It can be seen that gene expression changes after just two weeks of treatment. In the heatmap of FIG. 13B, 664 differentially expressed transcripts, between untreated active and latent TB patients in the untreated South Africa 2011 cohort, were obtained by twofold change from the median and stringent statistical filtering (Mann Whitney, Bonferroni p<0.01). The heatmap shows the dynamic change of gene expression in response to treatment in the treated South Africa 2011 cohort normalised to the median of all transcripts. In previously presented FIG. 2C, Ingenuity Pathway Analysis (IPA) of the 664 transcripts shows the top significant pathways. In FIG. 2D, an Interferon signaling pathway from the 664 list in IPA is shown. In FIG. 13C, weighted molecular distance to health (MDTH) of a treated South Africa 2011 cohort shows the signature significantly diminishes over time (linear mixed models, bars represent median & IQR, ***=p<0.001, **=p<0.01, *=p<0.05). As shown in FIG. 13D, temporal molecular response further shows significant and early changes in response to anti-TB treatment (linear mixed models, bars represent mean & 95% confidence intervals). See also FIG. 2 at doi:10.1371/journal.pone.0046191.g002 (Bloom et al. 2012).

FIGS. 14A-14B. Individual patient's transcriptional response occurred at a variable rate. For 320 gene list, differentially expressed genes derived from comparing the untreated expression profiles and their corresponding end-of-treatment (six months) expression profiles in the South Africa 2011 Active TB Training Set are evidenced. Heatmap of FIG. 14A is of South Africa 2011 cohort Active TB Test Set and shows hierarchical clustered transcripts normalised to the median of all transcripts, differentiating over time per individual. Diagrams of FIG. 14B illustrate each patient's temporal molecular response in the South Africa 2011 cohort Active TB Test Set. See also Supporting FIG. 2 at doi:10.1371/journal.pone.0046191.g001 (Bloom et al. 2012).

FIG. 15. The changing transcriptional response is independent of the magnitude of the untreated transcriptional signature. MDTH has been shown to correlate with radiological extent of active TB disease (see ref. [11] of Example 2). The magnitude of the patient's temporal molecular response during treatment, at both two weeks and two months, did not correlate with the magnitude of their untreated transcriptional signature, as evidenced measured by MDTH (linear regression r²<0.25, p>0.01). However, the patient's temporal molecular response after treatment, at six months and 12 months, did significantly correlate with his or her untreated MDTH (linear regression r²=0.32, p=0.003 and r²=0.38, p=0.0004, respectively). See also Supporting FIG. 1 at doi:10.1371/journal.pone.0046191.g001 (Bloom et al. 2012).

FIGS. 16A-16F. Specific treatment response signature significantly diminishes at two weeks and onwards. A specific TB treatment response signature of 320 transcripts was derived from significantly differentially expressed genes between untreated samples in the South Africa Active TB Training Set and their corresponding six month samples. Heatmap of FIG. 16A represents South Africa 2011 Active TB Training Set, normalised to the median of all transcripts, and shows transcripts differentiating over time in response to treatment. FIG. 16B displays corresponding temporal molecular response that further shows significant and early changes in response to TB treatment in the Active TB Training Set (linear mixed models, bars represent mean & 95% confidence intervals, ***=p<0.001, **=p<0.01, *=p<0.05). Heatmap of FIG. 16C represents South Africa 2011 Active TB Test Set, normalised to the median of all transcripts, and shows transcripts differentiating over time in response to treatment. FIG. 16D displays corresponding temporal molecular response that also shows in the Active TB Test Set significant and early changes in response to TB treatment. FIG. 16E summarizes IPA of the 320 transcripts showing the most significant pathways. FIG. 16F is a Venn diagram showing many overlapping genes between the active TB 664-transcript signature and the treatment specific 320-signature. See also FIG. 3 at doi:10.1371/journal.pone.0046191.g003 (Bloom et al. 2012).

FIGS. 17A-17B. Individual patient's transcriptional response occurred at a variable rate. FIGS. 17-17B concerns the 320 gene list and differentially expressed genes derived from comparing the untreated expression profiles and their corresponding end-of-treatment (six months) expression profiles in a South Africa 2011 Active TB Training Set. Heatmap of FIG. 17A is of South Africa 2011 cohort Active TB Training Set, normalised to the median of all transcripts, and shows hierarchical clustered transcripts differentiating over time per individual. For FIG. 17B, each patient's temporal molecular response diminishes in the Active TB Training Set cohort. See also FIG. 4 at doi:10.1371/journal.pone.0046191.g004 (Bloom et al. 2012).

FIGS. 18A-18C. Change in treatment specific signature is validated in an independent UK cohort. FIGS. 18A-18C concern the 320 gene list and differentially expressed genes between the untreated and six-month treated samples in the treated South Africa 2011 cohort. Heatmap of FIG. 18A is of the treated UK 2011 Cohort, normalised to the median of all transcripts, and shows diminution of the treatment specific transcriptional signature in the UK cohort in response to successful anti-TB treatment. For FIG. 18B, the temporal molecular response shows significant changes at two weeks in the UK cohort (linear mixed models, bars represent mean & 95% confidence intervals, ***=p<0.001, **=p<0.01, *=p<0.05). In FIG. 18C, a diminished response can be seen in each patient by their temporal molecular response. See also FIG. 5 at doi:10.1371/journal.pone.0046191.g005 (Bloom et al. 2012).

FIGS. 19, 20, and 21 provide heatmaps and corresponding temporal molecular response data for Patient ID 2208, Patient ID 2220, and Patient ID 2232, respectively, for 320, 86, and 393 transcript lists. FIGS. 19, 20, and 21 also provide summaries of clinical symptoms for Patient ID 2208, Patient ID 2220, and Patient ID 2232, respectively.

Table 1 lists genes present in the top significantly represented canonical pathways of Ingenuity Pathway Analysis in the 664 transcript list from an Untreated South Africa 2011 Cohort.

Summaries of demographics and clinical data are provided in Table 2A, a South African 2011 cohort, and Table 2B, a UK 2011 cohort.

Table 3 lists genes present in the top significantly represented canonical pathways of Ingenuity Pathway Analysis in active TB transcriptional signatures of 2009 UK and South Africa cohorts.

Tables 4 through 12 provide gene transcript lists for 224, 86, 393, 565, 664, 391, 1129, 711, and 320 genes, respectively.

Related summaries of demographics and clinical data are provided in Table 13A, a South African 2011 cohort, and Table 13B, a UK 2011 cohort.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate understanding of this invention, a number of terms are defined below. Terms defined herein have meanings as commonly understood by a person of ordinary skill in the areas relevant to the present invention. Terms such as “a”, “an,” and “the” are not intended to refer to only a singular entity but include the general class of which a specific example may be used for illustration. The terminology herein is used to describe specific embodiments of the invention, but their usage does not delimit the invention, except as outlined in the claims.

Globally there are approximately nine million new active tuberculosis (TB) cases and 1.7 million deaths annually. Effective anti-TB treatment monitoring is difficult as determining a treatment response by currently used methods takes at least two months. Inadequate treatment leads to worsening disease, disease transmission, and drug resistance. Currently, the best accepted method to predict treatment success in pulmonary tuberculosis is the two-month sputum culture conversion. However, this method is of low sensitivity for prediction of individual treatment response and is difficult to implement since many patients cannot produce sputum. In the United States 30% of TB patients are treated empirically and in South Africa 50% are treated with confirmation of diagnosis by culture. Currently, no recognized biomarkers of poor adherence or inadequate treatment earlier than two months exist.

The present inventors determined if blood transcriptional signatures change in response to anti-TB treatment and could act as biomarkers of a successful response.

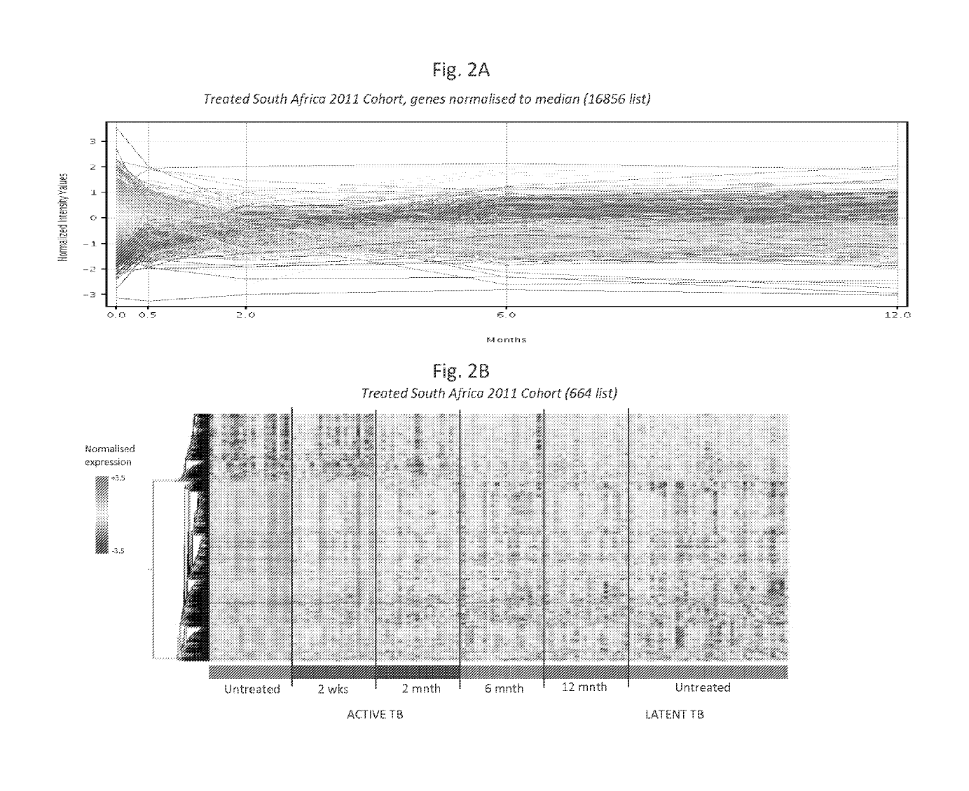

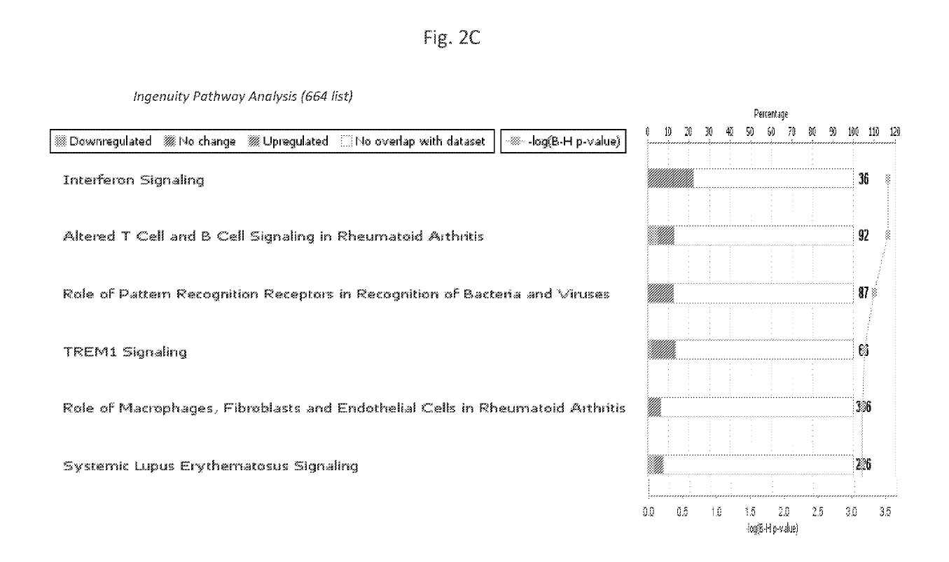

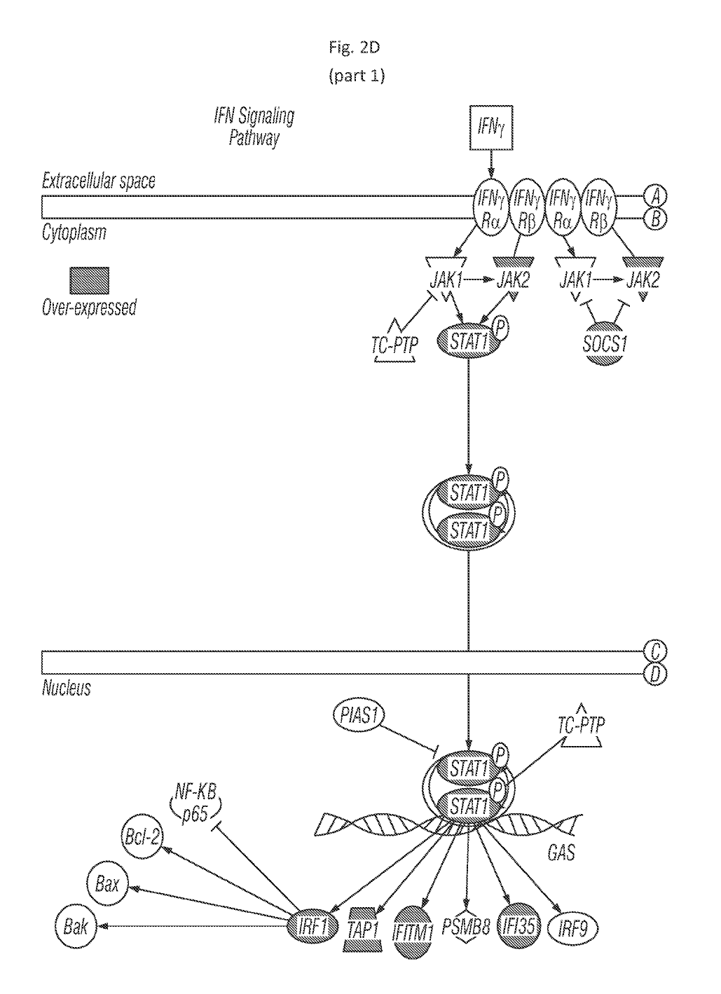

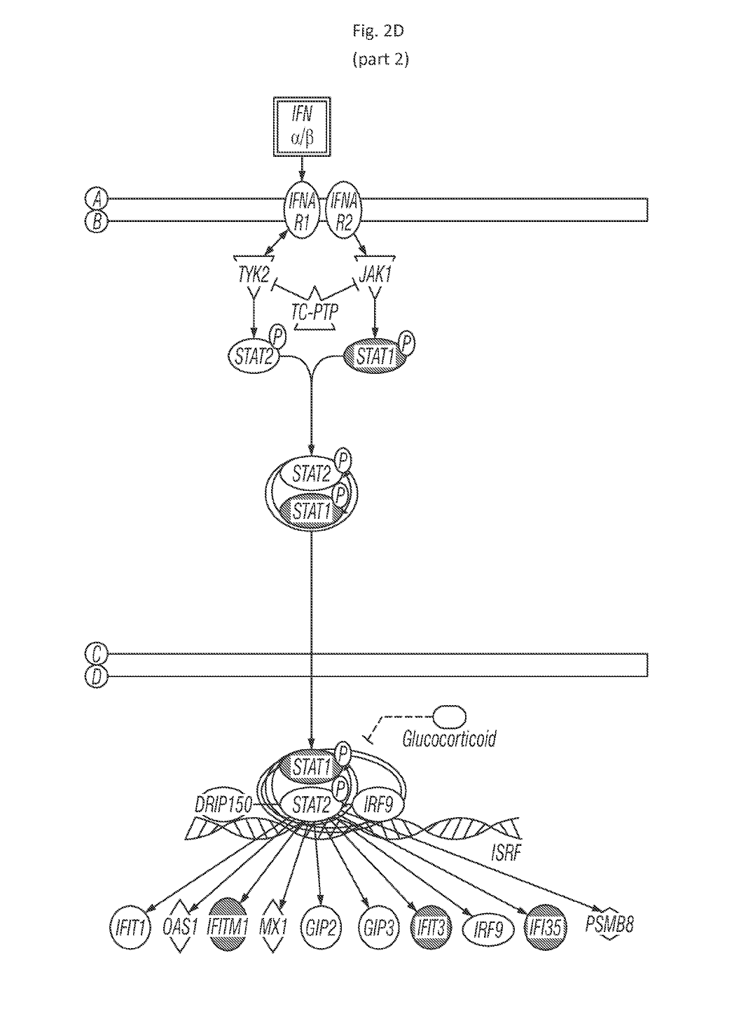

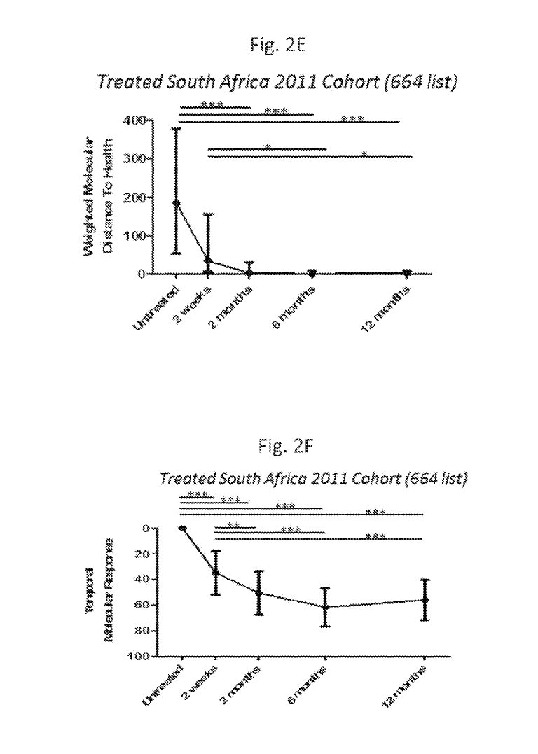

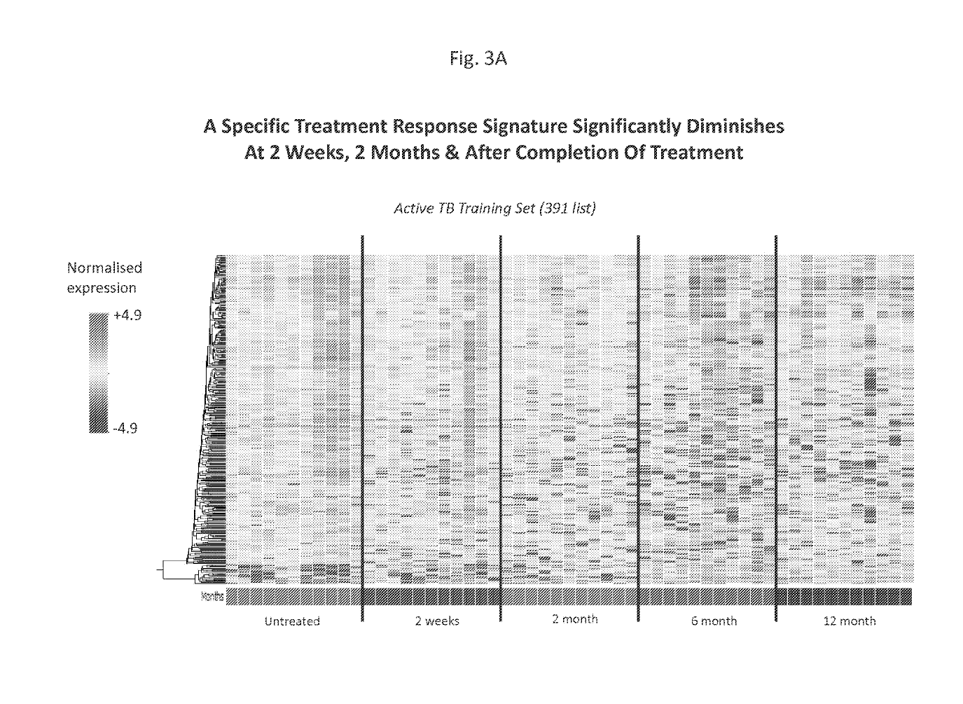

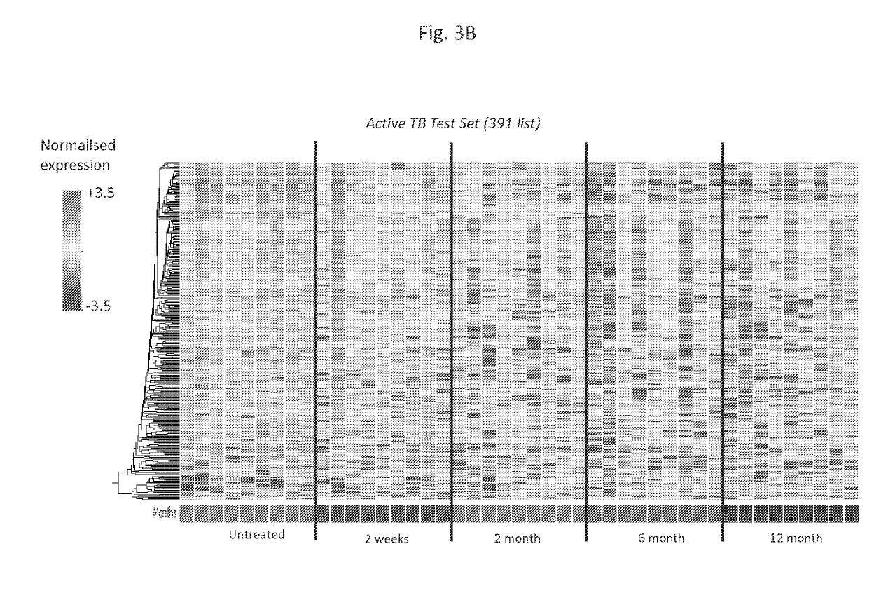

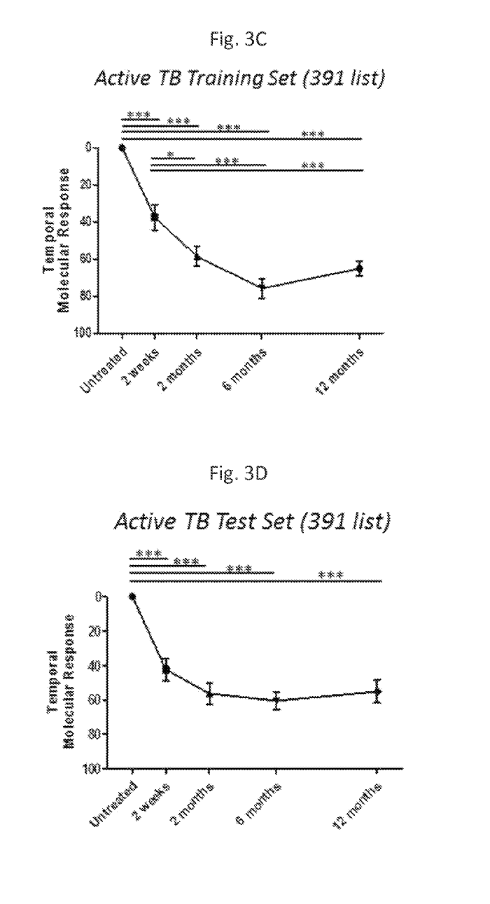

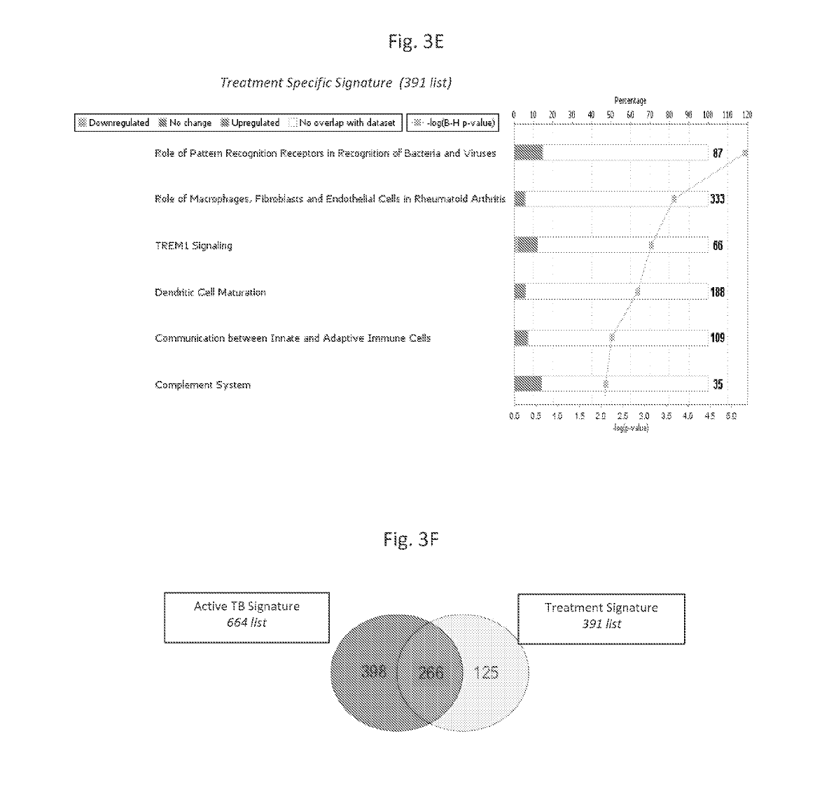

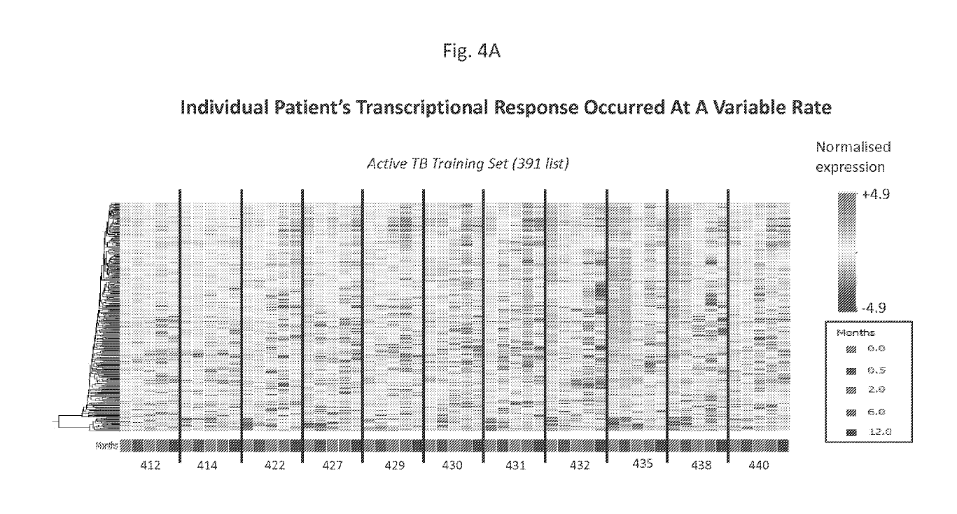

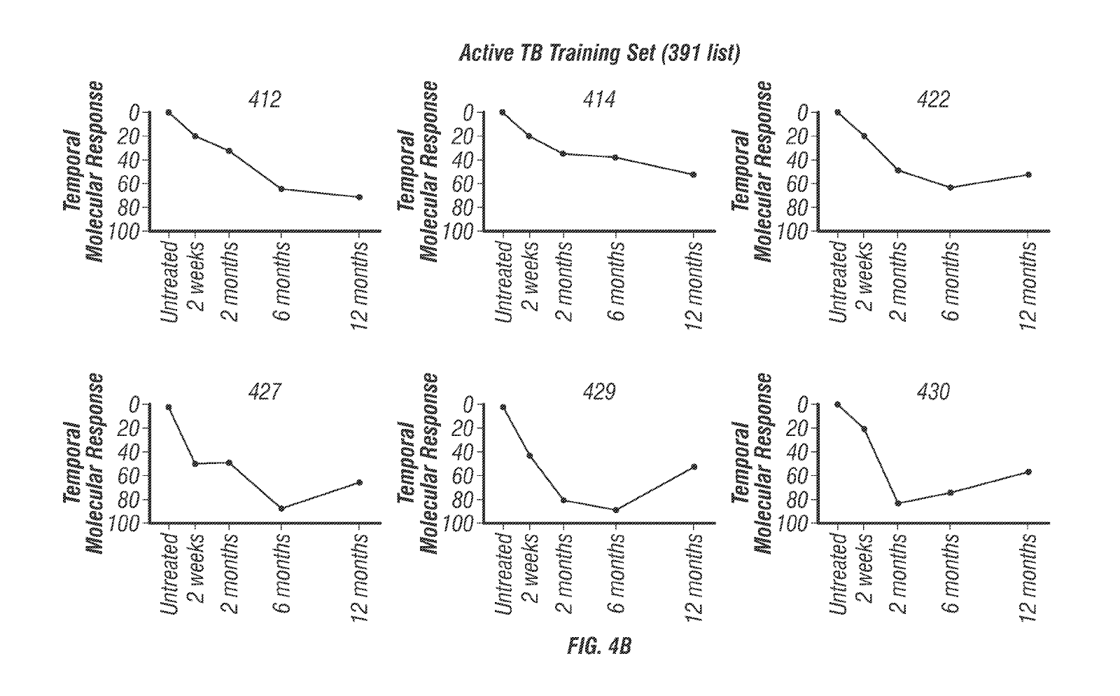

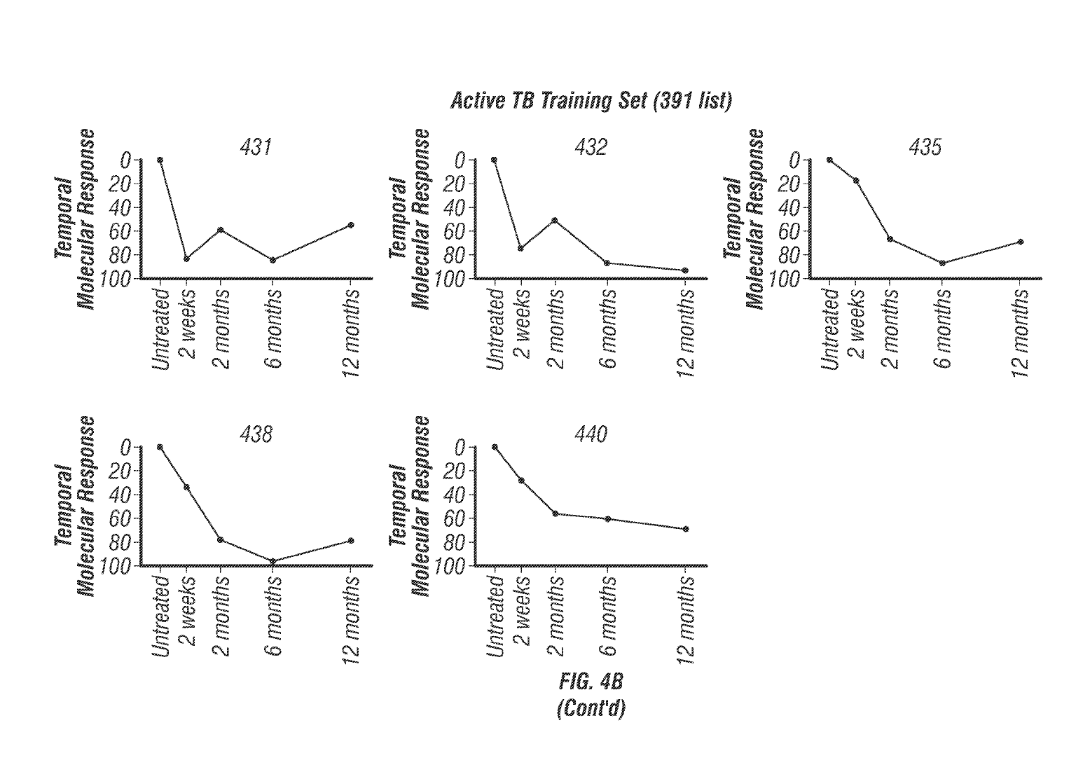

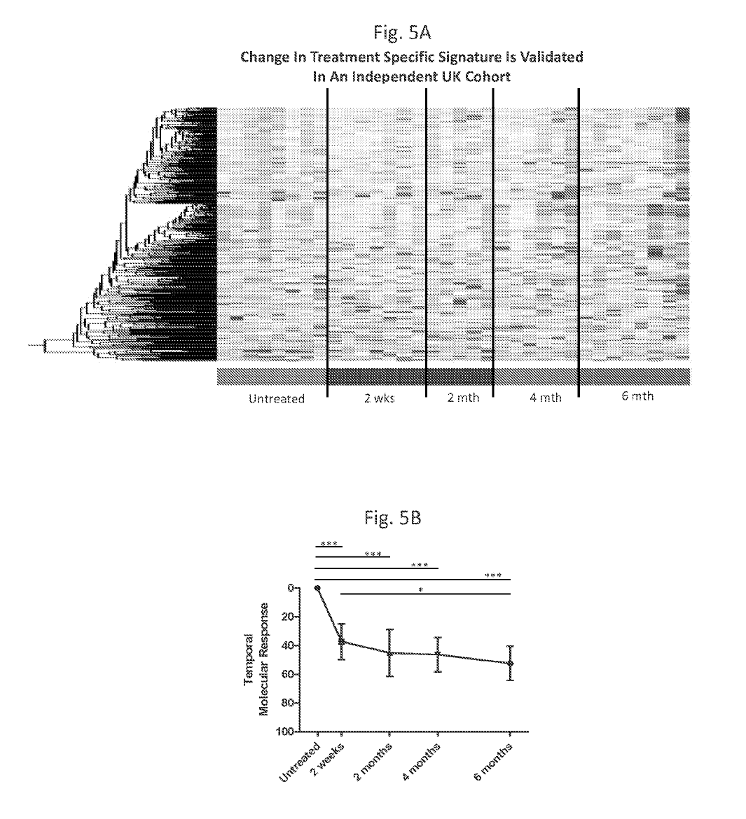

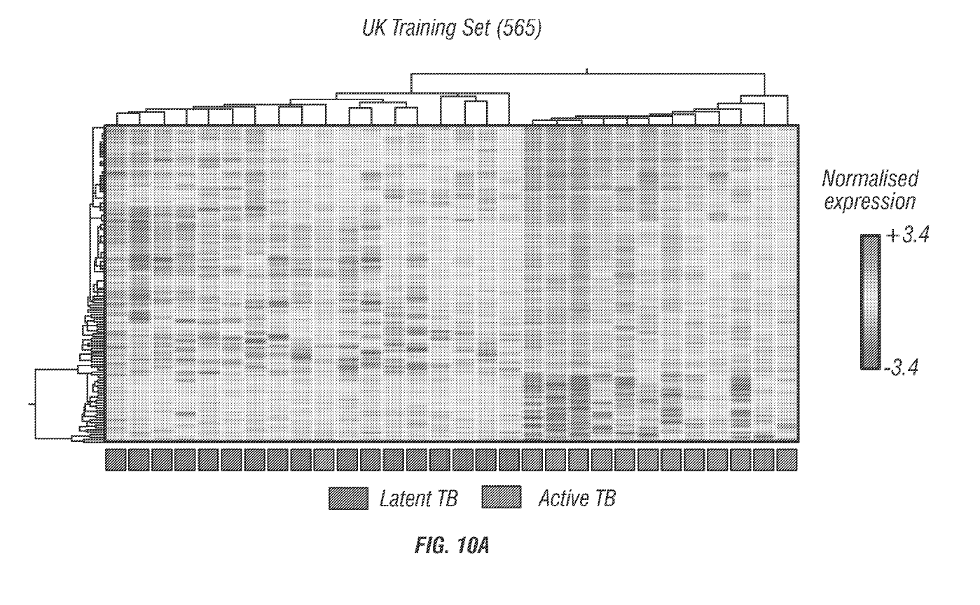

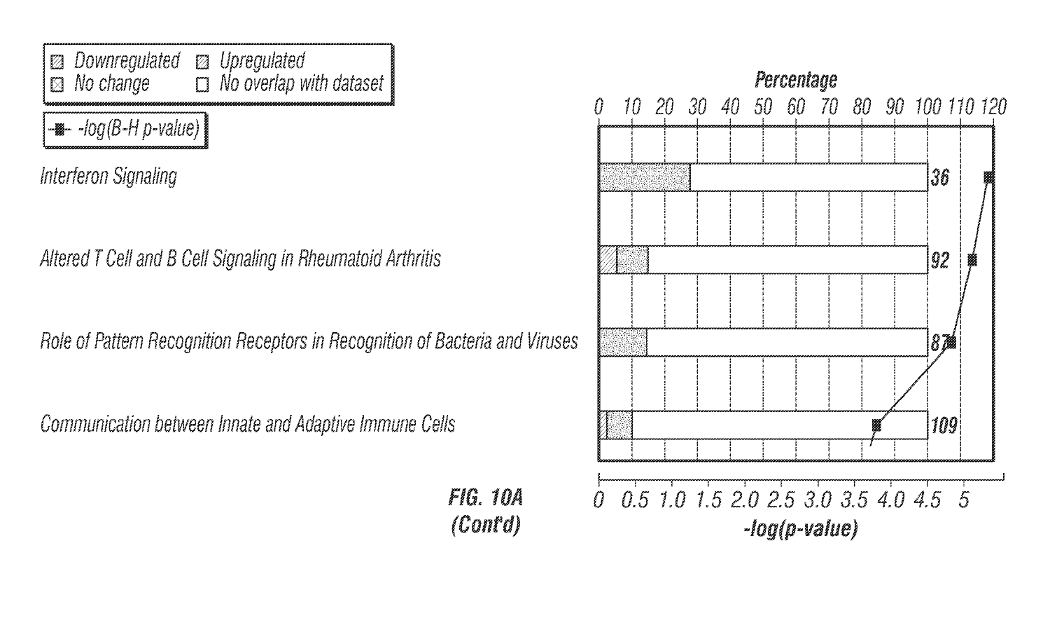

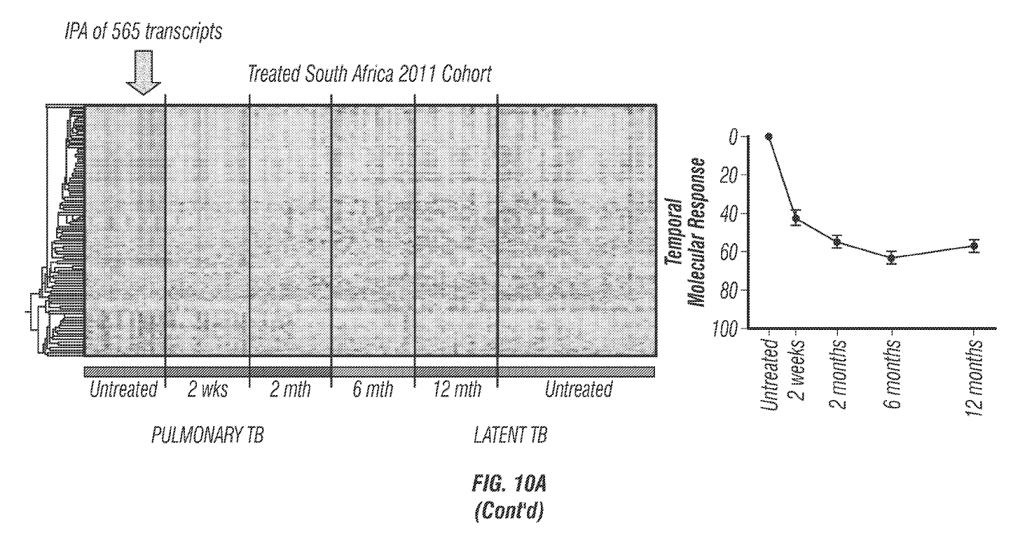

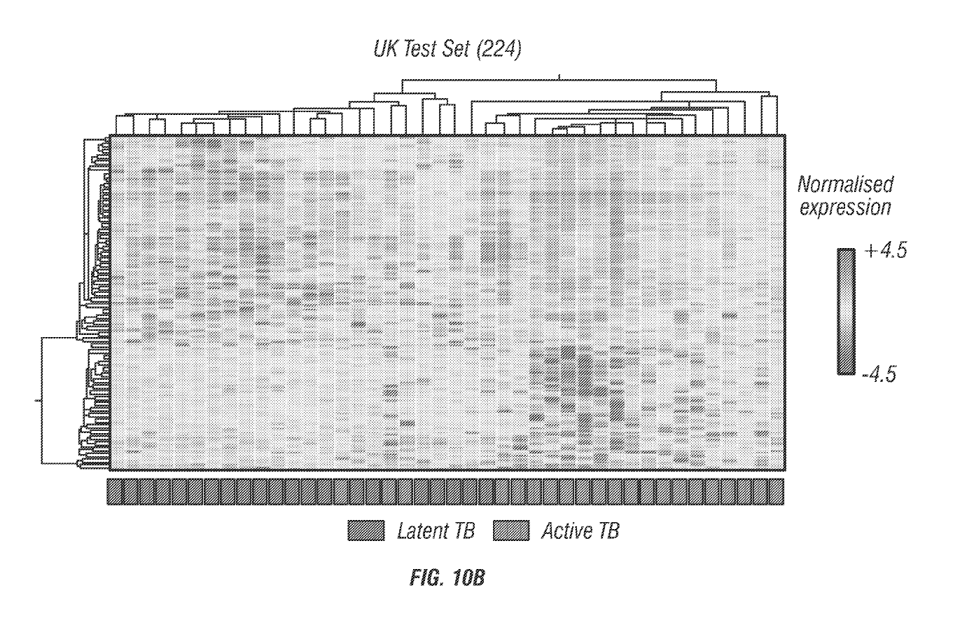

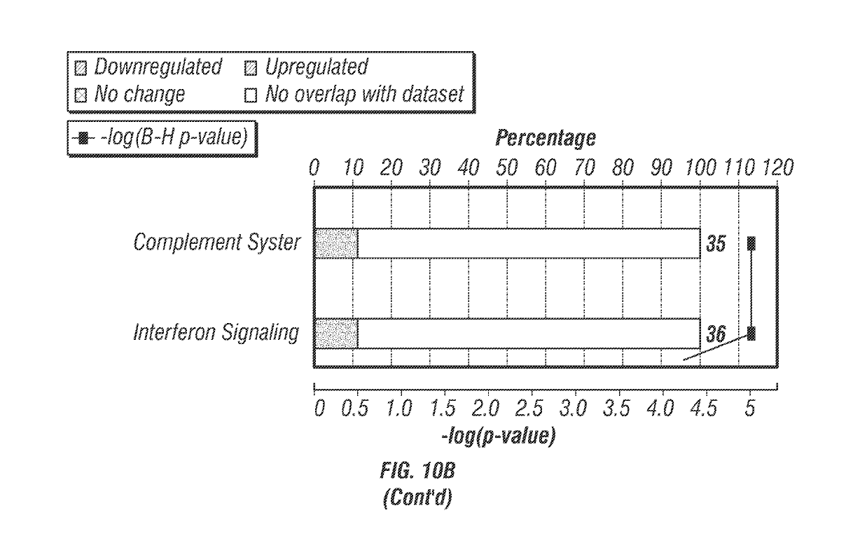

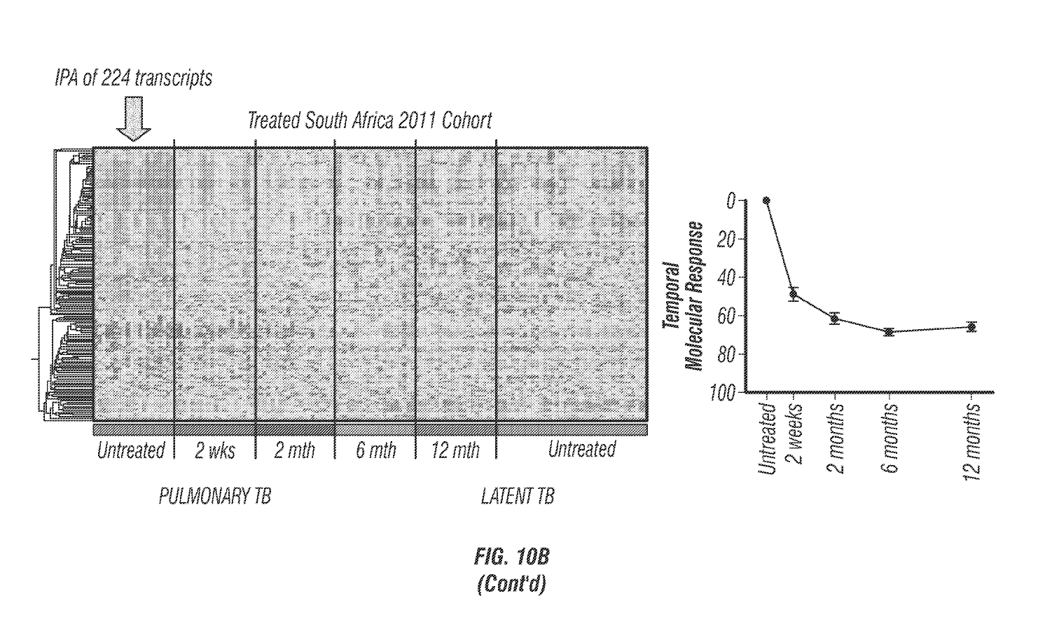

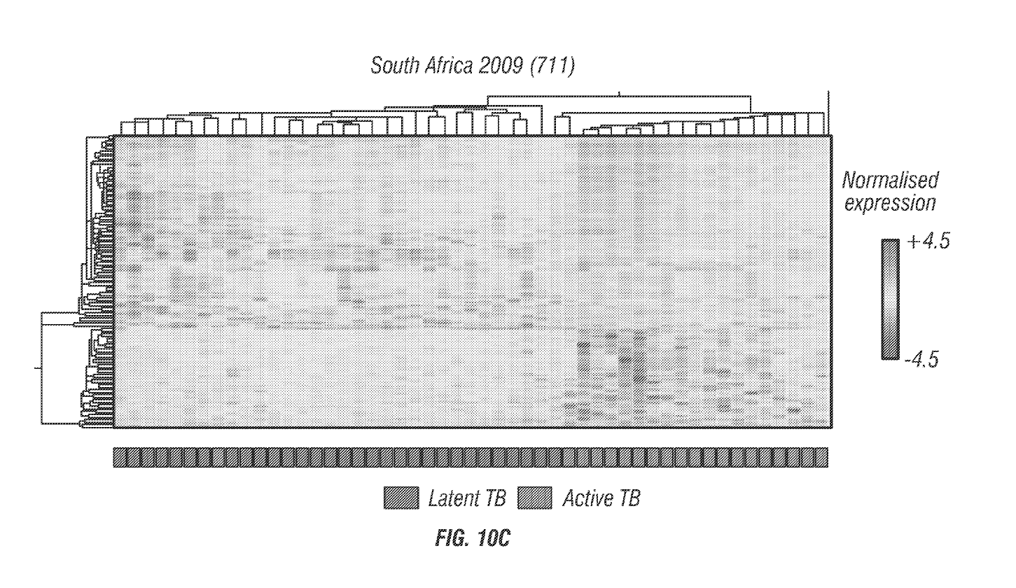

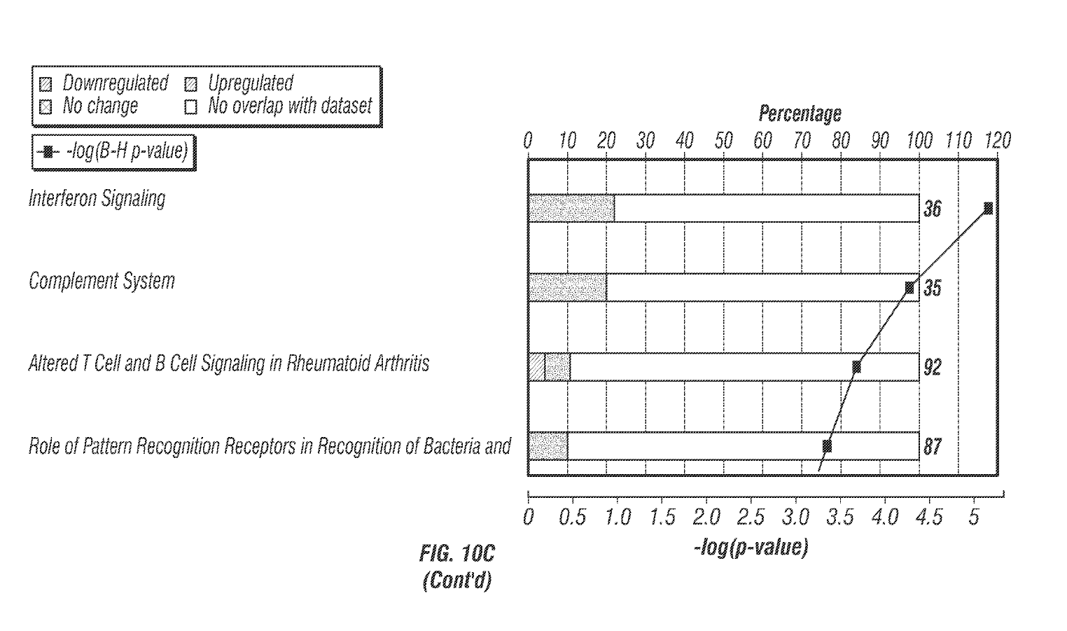

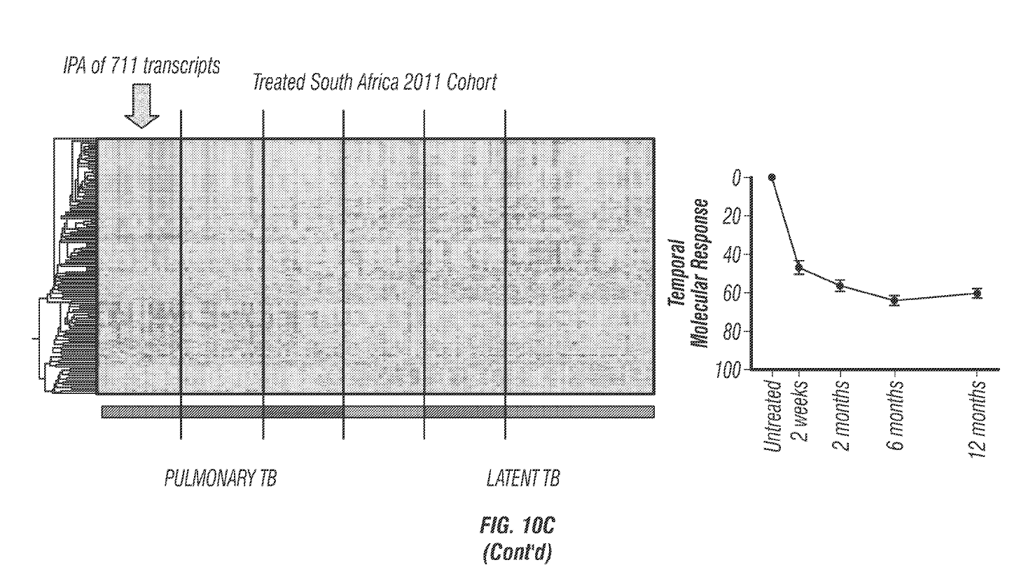

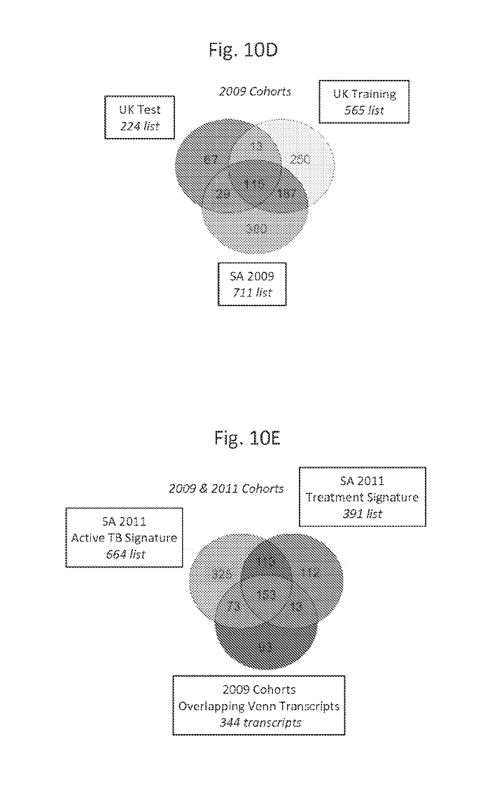

Surprisingly, transcriptional blood gene signatures {e.g.: a 664—(Table 8) (FIG. 2B); a 391—(Table 9) (e.g., FIGS. 3A, 4A & 7A); an 86—(Table 5) (FIGS. 8A & 8B); a 393—(Table 6) (FIGS. 8A & 8B); a 565—(Table 7) (FIG. 10A); a 224—(Table 4) (FIG. 10B); a 711—(Table 11) (FIG. 10C); or a 1129-transcript signature (Table 10)} diminish within two weeks after commencement of tuberculosis treatment; genes significantly altered in transcription include, e.g., interferon-signaling genes including type I and type II IFN, genes related to the innate immune pathways, genes related to complement, toll like receptors, a NOD like receptor gene, and interleukin-1B.

As demonstrated herein, a change in whole blood host transcriptional signatures is significantly detectable as early as two weeks or sooner after commencement of treatment for tuberculosis; this provides early biomarkers for treatment monitoring.

In short, blood transcriptional profiles of untreated active and latent TB patients in South Africa were analyzed, before, during (at two weeks and at two months), at the end of (six months) and after (12 months) anti-TB treatment. The signature in active TB patients as compared to latent individuals (664 transcripts) was significantly diminished by two weeks after initiation of treatment and this significant response was measured using a novel algorithm (termed “Temporal Molecular Responses”) developed for this study. A specific treatment response-transcriptional signature (391 transcripts) was derived and validated in two independent cohorts, to which two quantitative scoring algorithms were applied to score the changes in the transcriptional response. The most significantly represented pathways were determined using Ingenuity Pathway Analysis. The South African active TB-transcriptional signature revealed more differentially expressed genes than previously reported in UK cohorts. Interferon inducible genes were highly significantly elevated in all cohorts. The active TB-transcriptional signatures and the treatment specific transcriptional-signature significantly diminished after two weeks of treatment and continued to diminish significantly until six months. Significant changes in the transcriptional signatures measured by blood tests were readily detectable just two weeks after treatment initiation. Therefore transcriptional responses provide a clinical tool for monitoring an individual TB patient's response to treatment.

As used herein, a “significant change” between gene expression datasets is indicative that treatment is effective; in contrast, treatment is ineffective if less than a significant change between the first gene expression dataset and the second gene expression dataset is detected. A significant change can be determined by a person of ordinary skill in the art upon viewing a clearly visible change in transcriptional response using a heatmap or time-scaled profile plot of normalized intensity values or a simple time-scaled line graph of the transcriptional signature between the first and second time point. In some embodiments, the significant change can be determined upon generating a simple time-scaled line graph (also called a profile plot) of normalized signal intensity values. See, for example, FIG. 2A. Further embodiments determine a significant change by employing heatmaps. This is shown in FIGS. 2B, 3A, 3C, 4A, 5A, 7A, 8A, 8B, and 10A-C. The heatmaps are ordered either by time point or by each participant and may also show normalized intensity values. In yet further embodiments, the significant change is determined by line graphs showing molecular distance to health (MDTH), as shown in FIG. 2E. In addition the significant change can be determined and described via a temporal molecular response algorithm, as provided for in FIGS. 2F, 3B, 3D, 4B, 5B, 5C, 7B, 8C, 10A-C. To statistically demonstrate and describe a significant change, a conventional level of significance of 5% (only a 5% chance this change would occur by chance=p<0.05) may be employed. As non-limiting example upon testing more than three patients, a statistical significance value of p<0.05 between the first and second time point's temporal molecular response can be employed. In other examples, the number of patients examined may, for example, be eight and the p value<0.001. When applying the temporal molecular response algorithm to all participants at all the time points, the present inventors demonstrated that the participants' temporal molecular response at two weeks was statistically significant compared to their pre-treatment temporal molecular response. In addition, at all time points after two weeks, all participants' response continued to improve and the actual results showed that there was merely a 0.1% chance that this change occurred by chance. In some embodiments a significant change is determined for a single individual (independently from results from other individuals). In certain aspects, a percentage using the temporal molecular response is determined. The percentage reflects the percentage of genes that are changing over time relative to the transcriptional signature being tested. This enhances the ability to monitor individual patients in hospitals/clinics. In certain aspects, 19% or more correlates with a good treatment response and constitutes a significant change. In other aspects, any value above 10% correlates with a good treatment response and constitutes a significant change. In some embodiment, an individual's temporal molecular response value of greater than 15% difference between the first and second time point constitutes a significant change.

As used herein, the term “array” refers to a solid support or substrate with one or more peptides or nucleic acid probes attached to the support. Arrays typically have one or more different nucleic acid or peptide probes that are coupled to a surface of a substrate in different, known locations. These arrays, also described as “microarrays” or “gene-chips” that may have 10,000; 20,000, 30,000; or 40,000 different identifiable genes based on the known genome, e.g., the human genome. These pan-arrays are used to detect the entire “transcriptome” or transcriptional pool of genes that are expressed or found in a sample, e.g., nucleic acids that are expressed as RNA, mRNA and the like that may be subjected to RT and/or RT-PCR to made a complementary set of DNA replicons. Arrays may be produced using mechanical synthesis methods, light directed synthesis methods and the like that incorporate a combination of non-lithographic and/or photolithographic methods and solid phase synthesis methods.

Various techniques for the synthesis of these nucleic acid arrays have been described, e.g., fabricated on a surface of virtually any shape or even a multiplicity of surfaces. Arrays may be peptides or nucleic acids on beads, gels, polymeric surfaces, fibers such as fiber optics, glass or any other appropriate substrate. Arrays may be packaged in such a manner as to allow for diagnostics or other manipulation of an all-inclusive device, see for example, U.S. Pat. No. 6,955,788, which is incorporated herein by reference in its entirety.

As used herein, the term “disease” refers to a physiological state of an organism with any abnormal biological state of a cell. Disease includes, but is not limited to, an interruption, cessation or disorder of cells, tissues, body functions, systems or organs that may be inherent, inherited, caused by an infection, caused by abnormal cell function, abnormal cell division and the like. A disease that leads to a “disease state” is generally detrimental to the biological system, that is, the host of the disease. With respect to the present invention, any biological state, such as an infection (e.g., viral, bacterial, fungal, helminthic, etc.), inflammation, autoinflammation, autoimmunity, anaphylaxis, allergies, premalignancy, malignancy, surgical, transplantation, physiological, and the like that is associated with a disease or disorder is considered to be a disease state. A pathological state is generally the equivalent of a disease state.

Disease states may also be categorized into different levels of disease state. As used herein, the level of a disease or disease state is a measure reflecting the progression of a disease or disease state as well as the physiological response upon, during and after treatment. Generally, a disease or disease state will progress through levels or stages, wherein the effects of the disease become increasingly severe. The level of a disease state may be impacted by the physiological state of cells in the sample.

As used herein, the terms “therapy” or “therapeutic regimen” refer to those medical steps taken to alleviate or alter a disease state, e.g., a course of treatment intended to reduce or eliminate the affects or symptoms of a disease using pharmacological, surgical, dietary and/or other techniques. A therapeutic regimen may include a prescribed dosage of one or more drugs or surgery. Therapies will most often be beneficial and reduce the disease state but in many instances the effect of a therapy will have non-desirable or side-effects. The effect of therapy will also be impacted by the physiological state of the host, e.g., age, gender, genetics, weight, other disease conditions, etc.

As used herein, the term “pharmacological state” or “pharmacological status” refers to those samples from diseased individuals that will be, are and/or were treated with one or more drugs, surgery and the like that may affect the pharmacological state of one or more nucleic acids in a sample, e.g., newly transcribed, stabilized and/or destabilized as a result of the pharmacological intervention. The pharmacological state of a sample relates to changes in the biological status before, during and/or after drug treatment and may serve as a diagnostic or prognostic function, as taught herein. Some changes following drug treatment or surgery may be relevant to the disease state and/or may be unrelated side-effects of the therapy. Changes in the pharmacological state are the likely results of the duration of therapy, types and doses of drugs prescribed, degree of compliance with a given course of therapy, and/or un-prescribed drugs ingested.

As used herein, the term “biological state” refers to the state of the transcriptome (that is the entire collection of RNA transcripts) of the cellular sample isolated and purified for the analysis of changes in expression. The biological state reflects the physiological state of the cells in the blood sample by measuring the abundance and/or activity of cellular constituents, characterizing according to morphological phenotype or a combination of the methods for the detection of transcripts.

As used herein, the term “expression profile” refers to the relative abundance of RNA, DNA abundances or activity levels. The expression profile can be a measurement for example of the transcriptional state or the translational state by any number of methods and using any of a number of gene-chips, gene arrays, beads, multiplex PCR, quantitative PCR, run-on assays, Northern blot analysis, or using RNA-seq, nanostring, nanopore RNA sequencing etc. Apparatus and system for the determination and/or analysis of gene expression that are readily commercially available.

As used herein, the term “transcriptional state” of a sample includes the identities and relative abundances of the RNA species, especially mRNAs present in the sample. The entire transcriptional state of a sample, that is the combination of identity and abundance of RNA, is also referred to herein as the transcriptome. Generally, a substantial fraction of all the relative constituents of the entire set of RNA species in the sample are measured.

Regarding the “expression level,” the group comparison for a given disease provides the list of differentially expressed transcripts. It was found that different diseases yield different subsets of gene transcripts.

Using the present invention it is possible to determine the effectiveness of a treatment for tuberculosis at the gene-level; i.e., two diseases can have the same vector (identical proportion of differentially expressed transcripts, identical “polarity”), but the gene composition of the vector can still be disease-specific. Gene-level expression provides the distinct advantage of greatly increasing the resolution of the analysis. Furthermore, the present invention takes advantage of composite transcriptional markers.

As used herein, the term “composite transcriptional markers” refers to the average expression values of multiple genes (composite of transcripts) as compared to using individual genes as markers (and the composition of these markers can be disease-specific). The composite transcriptional markers approach is unique because the user can develop multivariate microarray scores to assess disease severity in patients with, e.g., tuberculosis (TB) or systemic lupus erythematosus (SLE), or to derive expression vectors disclosed herein. It has been found that using the composite transcriptional markers of the present invention the results found herein are reproducible across microarray platform, thereby providing greater reliability for regulatory approval.

Gene expression monitoring systems for use with the present invention may include customized gene arrays with a limited and/or basic number of genes that are specific and/or customized for the one or more target diseases. Unlike the general, pan-genome arrays that are in customary use, the present invention provides for not only the use of these general pan-arrays for retrospective gene and genome analysis without the need to use a specific platform, but more importantly, it provides for the development of customized arrays that provide an optimal gene set for analysis without the need for the thousands of other, non-relevant genes. One distinct advantage of the optimized arrays and gene sets of the present invention over the existing art is a reduction in the financial costs (e.g., cost per assay, materials, equipment, time, personnel, training, etc.), and more importantly, the environmental cost of manufacturing pan-arrays where the vast majority of the data is irrelevant. By reducing the total number of genes for analysis, or eliminating genes for analysis, it is possible to, e.g., eliminate the need to manufacture thousands of expensive platinum masks for photolithography during the manufacture of pan-genetic chips that provide vast amounts of irrelevant data. Using the present invention it is possible to completely avoid the need for microarrays if the limited probe set(s) of the present invention are used with, e.g., digital optical chemistry arrays, ball bead arrays, multiplex PCR, quantitative PCR, “RNA-seq” for measuring mRNA levels using next-generation sequencing technologies, nanostring-type technologies or any other method, apparatus and system for the determination and/or analysis of gene expression that are readily commercially available.

The “molecular fingerprinting system” of the present invention may be used to facilitate and conduct a comparative analysis of expression in different cells or tissues, different subpopulations of the same cells or tissues, different physiological states of the same cells or tissue, different developmental stages of the same cells or tissue, or different cell populations of the same tissue against other diseases and/or normal cell controls. In some cases, the normal or wild-type expression data may be from samples analyzed at or about the same time or it may be expression data obtained or culled from existing gene array expression databases, e.g., public databases such as the NCBI Gene Expression Omnibus database.

As used herein, the term “differentially expressed” refers to the measurement of a cellular constituent (e.g., nucleic acid, protein, enzymatic activity and the like) that varies in two or more samples, e.g., between a disease sample and a normal sample. The cellular constituent may be on or off (present or absent), upregulated relative to a reference or downregulated relative to the reference. For use with gene-chips or gene-arrays, differential gene expression of nucleic acids, e.g., mRNA or other RNAs (miRNA, siRNA, hnRNA, rRNA, tRNA, etc.) may be used to distinguish between cell types or nucleic acids. Most commonly, the measurement of the transcriptional state of a cell is accomplished by quantitative reverse transcriptase (RT) and/or quantitative reverse transcriptase-polymerase chain reaction (RT-PCR), genomic expression analysis, post-translational analysis, modifications to genomic DNA, translocations, in situ hybridization and the like.

The skilled artisan will appreciate readily that samples may be obtained from a variety of sources including, e.g., single cells, a collection of cells, tissue, cell culture and the like. In certain cases, it may even be possible to isolate sufficient RNA from cells found in, e.g., urine, blood, saliva, tissue or biopsy samples and the like. In certain circumstances, enough cells and/or RNA may be obtained from: mucosal secretion, feces, tears, blood plasma, peritoneal fluid, interstitial fluid, intradural, cerebrospinal fluid, sweat or other bodily fluids. The nucleic acid source, e.g., from tissue or cell sources, may include a tissue biopsy sample, one or more sorted cell populations, cell culture, cell clones, transformed cells, biopsies or a single cell. The tissue source may include, e.g., brain, liver, heart, kidney, lung, spleen, retina, bone, neural, lymph node, endocrine gland, reproductive organ, blood, nerve, vascular tissue, and olfactory epithelium.

The present invention includes the following basic components, which may be used alone or in combination, namely, one or more data mining algorithms, one a novel algorithm specifically developed for this TB treatment monitoring, the Temporal Molecular Response; the characterization of blood leukocyte transcriptional gene sets; the use of aggregated gene transcripts in multivariate analyses for the molecular diagnostic/prognostic of human diseases; and/or visualization of transcriptional gene set-level data and results. Using the present invention it is also possible to develop and analyze composite transcriptional markers. The composite transcriptional markers for individual patients in the absence of control sample analysis may be further aggregated into a reduced multivariate score.

An explosion in data acquisition rates has spurred the development of mining tools and algorithms for the exploitation of microarray data and biomedical knowledge. Approaches aimed at uncovering the function of transcriptional systems constitute promising methods for the identification of robust molecular signatures of disease. Indeed, such analyses can transform the perception of large-scale transcriptional studies by taking the conceptualization of microarray data past the level of individual genes or lists of genes.

The present inventors have recognized that current microarray-based research is facing significant challenges with the analysis of data that are notoriously “noisy,” that is, data that are difficult to interpret and do not compare well across laboratories and platforms. A widely accepted approach for the analysis of microarray data begins with the identification of subsets of genes differentially expressed between study groups. Users may try subsequently to “make sense” out of resulting gene lists using standard algorithms and existing scientific knowledge and by validating in independent sample sets and in different microarray analyses.

### Example 1

Pulmonary tuberculosis (PTB) is a major and increasing cause of morbidity and mortality worldwide caused by Mycobacterium tuberculosis (M. tuberculosis). However, the majority of individuals infected with M. tuberculosis remain asymptomatic, retaining the infection in a latent form, and it is thought that this latent state is maintained by an active immune response. Blood is the pipeline of the immune system, and as such it is the ideal biologic material from which the health and immune status of an individual can be established.

Blood represents a reservoir and a migration compartment for cells of the innate and the adaptive immune systems, including either neutrophils, dendritic cells and monocytes, or B and T lymphocytes, respectively, which during infection will have been exposed to infectious agents in the tissue. For this reason whole blood from infected individuals provides an accessible source of clinically relevant material where an unbiased molecular phenotype can be obtained using gene expression microarrays as previously described for the study of cancer in tissues (Alizadeh A A., 2000; Golub, T R., 1999; Bittner, 2000), and autoimmunity (Bennet, 2003; Baechler, E C, 2003; Burczynski, M E, 2005; Chaussabel, D., 2005; Cobb, J P., 2005; Kaizer, E C., 2007; Allantaz, 2005; Allantaz, 2007), and inflammation (Thach, D C., 2005) and infectious disease (Ramillo, Blood, 2007) in blood or tissue (Bleharski, J R et al., 2003). Microarray analyses of gene expression in blood leucocytes have identified diagnostic and prognostic gene expression signatures, which have led to a better understanding of mechanisms of disease onset and responses to treatment (Bennet, L 2003; Rubins, K H., 2004; Baechler, E C, 2003; Pascual, V., 2005; Allantaz, F., 2007; Allantaz, F., 2007). These microarray approaches have been attempted for the study of active and latent TB but as yet have yielded small numbers of differentially expressed genes only (Jacobsen, M., Kaufmann, S H., 2006; Mistry, R, Lukey, P T, 2007), and in relatively small numbers of patients (Mistry, R., 2007), which may not be robust enough to distinguish between other inflammatory and infectious diseases. The present inventors recognize that a neutrophil driven blood transcriptional signature in active TB patients was missing in the majority of latent TB individuals and in healthy controls. See, also (9). This signature of active TB was reflective of lung radiographic disease and was diminished after two months of treatment. The signature was dominated by interferon-inducible genes, and at a modular level the active TB signature was distinct from other infectious or autoimmune diseases.

To define an immune signature in TB, the blood of TB patients before and after commencement of treatment and controls were analyzed; patients were selected using very stringent clinical criteria.

Approximately one third of the world is infected with the pathogen Mycobacterium tuberculosis (Mtb), the cause of TB. While most remain asymptomatic, termed latent, approximately 10% develop active TB during their lifetime (1). Over nine million new cases of active TB and 1.7 million deaths annually have been reported (2). Improved diagnostics, more effective and shorter treatments than the current minimum of six months, and improvements in treatment monitoring are badly needed.

Active pulmonary TB diagnosis requires culture of Mtb, which may take up to six weeks (3). Conventional determination of antibiotic sensitivities demands several more weeks of culture. Mtb is isolated from sputum, which is often difficult to obtain, or from lung washings using invasive and expensive methods, which are prohibitive in developing countries. Due to insufficient samples and poor availability of culture, approximately 30% of patients in the USA and 50% of South African patients are treated empirically (2, 4). Although the World Health Organization (WHO) endorsed Xpert MTB/RIF automated molecular test for Mtb results in rapid diagnosis, this test still requires sputum (5). After diagnosis there are no available early biomarkers correlating with treatment success, resulting in significant delay in assessing treatment response. In poor responders this delay can result in worsening disease and spread of drug resistant bacteria. Currently sputum conversion to negative culture after two months of treatment is the only accepted biomarker (6). However a systematic review and meta-analysis to assess its accuracy to predict an individual's treatment failure revealed low sensitivity and modest specificity (7). Chest X-rays are commonly used to assess response but are not universally available and assessment is difficult to standardize (8). Lack of practicable treatment monitoring is concerning due to the development of multidrug resistant (MDR) and extensively drug resistant (XDR) TB, mainly caused by non-adherence or inappropriate drug regimens, resulting in a detrimental impact on global TB treatment programs.

A whole blood transcriptional signature can distinguish active TB from latent TB and other diseases, and be correlated with radiographic extent of disease (9). This active TB blood signature diminished after two months of successful treatment and reverted to that of healthy individuals after completing treatment (9). Early blood biomarkers correlating with treatment response will allow monitoring of patients without sputum, expedite knowledge of an individual's treatment response and may permit stratification of patients requiring differing treatment regimens. Furthermore early biomarkers can be instrumental in drug development.

Certain embodiments of the present invention are designed to establish that early changes in a blood transcriptional response can be observed during anti-TB treatment. Furthermore, it adds to previous results by examining the transcriptional treatment response directly in a larger cohort from a high-burden TB country, South Africa (2).

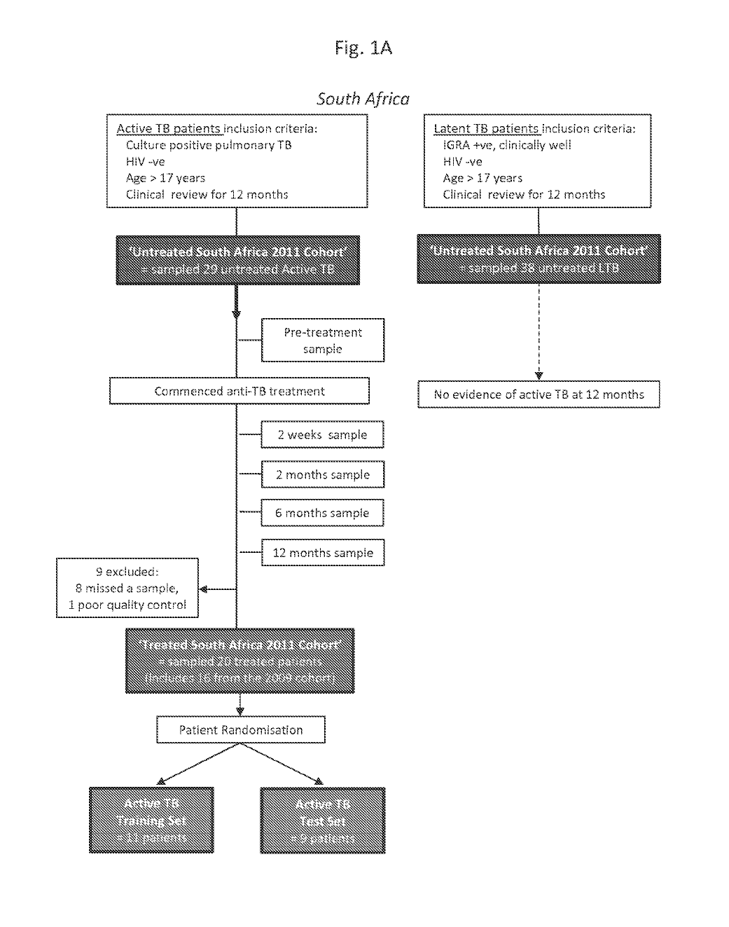

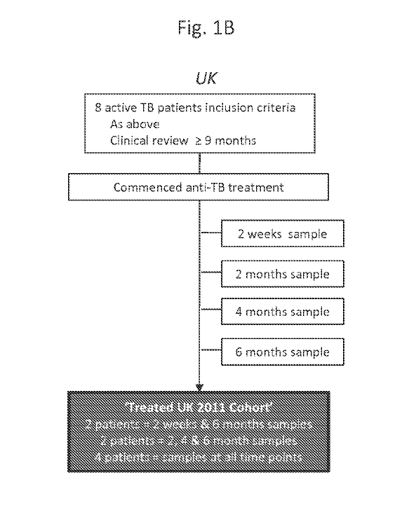

METHODS: Study Population: Blood was collected between May 2008-November 2011 in Ubuntu TB/HIV clinic, South Africa and Royal Free Hospital NHS Trust, London from patients (age>17 years) with Mtb culture positive active pulmonary TB (FIG. 1A; Table 2A,B). Latent TB patients were asymptomatic with a positive QuantiFERON-TB Gold In-Tube assay (Cellestis). South African active TB patients were sampled before treatment and at two weeks and two, six, and 12 months after treatment initiation. Response was assessed clinically. The UK 2011 TB patients were sampled before treatment and at two weeks and two, four and six months after treatment initiation. Chest X-rays were performed before and during treatment. The 2009 cohorts were as previously described (9).

Expression Profiling: The following were performed according to the manufacturer's instructions. Blood was collected into Tempus tubes (Applied Biosystems/Ambion). 2011 sample's RNA was isolated using MagMAX-96 Blood RNA Isolation Kit (Applied Biosystems/Ambion), globin reduced using GLOBINclear 96-well format kit (Applied Biosystems/Ambion), biotinylated, amplified antisense complementary RNA (cRNA) targets were prepared using Illumina CustomPrep RNA amplification kit (Applied Biosystems/Ambion). RNA integrity and yield were assessed using Agilent 2100 Bioanalyzer (Agilent Technologies) and NanoDrop 800 spectrophotometer (NanoDrop Products, Thermo Fisher Scientific), respectively. Labeled cRNA was hybridized to Illumina Human HT-12 V4 BeadChip arrays (Illumina) and scanned on an Illumina iScan. GenomeStudio (Illumina) was used for quality control and to generate signal intensity values. 2009 sample's RNA was processed as previously described (9). Using GeneSpring GX version 11.5 (Agilent Technologies) raw data were analyzed by the following: background subtraction, filtering by detection significance (p<0.01), threshold set, log 2 transformed, per-chip normalised (75th percentile shift algorithm) and per-gene normalised to median of latent TB samples.

Statistical Analysis: GeneSpring 11.5 was used to select transcripts with an expression fold change (active TB-signature: twofold expression from latent TB samples; treatment specific signature: threefold expression in 8/11 training set matched untreated and six-month treated samples). Statistical filtering was then applied using non-parametric tests and multiple testing corrections (Benjamini Hochberg or Bonferroni) (10, 11). The Treated South Africa 2011 cohort was randomised into a training and test set (12). Derived signatures were then applied to the: Treated South Africa 2011 cohort, Treated UK 2011 Cohort, and cohorts from the earlier Berry et al. (2010) study. Data was displayed in heatmaps generated by hierarchical clustering (distance metric: Pearson's uncentered with average linkage (13)) showing either clustering of transcripts and samples, or just clustering of transcripts.

Molecular distance to health (MDTH) was determined as previously described (14). In one embodiment, the Temporal Molecular Response was calculated from the sum of transcripts that were greater than twofold different between one time point and the baseline values, then expressed as a percentage of the total number of transcripts in that signature. MDTH and temporal molecular response were calculated in Microsoft Excel 2010. Graphs, p-values and linear regression were generated in GraphPad Prism version 5 for Windows except linear mixed models was performed in SASTM software (SAS Institute Inc., USA). Ingenuity Pathway Analysis (Ingenuity Systems, Inc., Redwood, Calif.), identified significant canonical pathways (Fisher's exact Benjamini Hochberg p<0.05).

Study Population and Inclusion Criteria: All participants in South Africa were recruited from the Ubuntu TB/HIV clinic in Khayelitsha, a large peri-urban African township in Cape Town which has over 1000 TB notifications annually. During the period May 2008-August 2010 whole blood was collected from adult patients (age>17 years) with drug sensitive Mtb culture proven active pulmonary TB (FIG. 1A). Due to the population's high Mtb exposure, controls were considered as asymptomatic individuals with previous exposure to Mtb (latent TB patients); exposure was evidenced by a positive QuantiFERON-TB Gold In-Tube (QFT). Participants with latent TB were recruited from individuals self-referring to the voluntary testing clinic. All participants had negative HIV status.

All participants in the 2009 UK Training and Test cohorts were selected as previously described (9). The UK 2011 Active TB Validation Cohort were all Mtb culture proven adults (>17 years) recruited between August 2009-November 2011 from the Royal Free Hospital, London (FIG. 1B). Clinical and demographic data was recorded for all participants and stored in a database.

Follow Up Period: All 20 Treated 2011 South Africa active TB patients completed a full six months of treatment. Each patient was sampled for venous blood at every time point: two weeks, two months, six months and 12 months after initiation of treatment (FIG. 1A). Patient's response to anti-TB treatment was assessed clinically during the 12-month period. All patients were discharged from the program as cured.

Eight Treated 2011 UK Active TB patients completed a full six months of treatment, one patient completed nine months of treatment due to radiographic uncertainty of treatment success. Each patient was sampled for venous blood at two weeks, two months, four months and six months after initiation of treatment (FIG. 1B). Four patients had a sample at every time point, three patients had samples at two, four and six months, and four patients had samples at two weeks and six months. As part of their routine medical care all patients had chest X-rays minimally at the beginning and end of their treatment and were discharged from the program as cured.

IFNγ Release Assay Testing: The QFT Assay (Cellestis) was performed according to the manufacturer's instructions.

Gene Expression Profiling: 3 ml of whole blood were collected into Tempus tubes (Applied Biosystems/Ambion) by standard phlebotomy, vigorously mixed immediately after collection, and stored between −20 and −80° C. before RNA extraction. South Africa and UK 2011 sample's RNA was isolated using 1.5 ml whole blood and the MagMAX-96 Blood RNA Isolation Kit (Applied Biosystems/Ambion) according to the manufacturer's instructions. 250 μg of isolated total RNA was globin reduced using the GLOBINclear 96-well format kit (Applied Biosystems/Ambion) according to the manufacturer's instructions. Total and globin-reduced RNA integrity was assessed using an Agilent 2100 Bioanalyzer (Agilent Technologies). RNA yield was assessed using a NanoDrop 800 spectrophotometer (NanoDrop Products, Thermo Fisher Scientific). Biotinylated, amplified antisense complementary RNA (cRNA) targets were then prepared from 200-250 ng of the globin-reduced RNA using the Illumina CustomPrep RNA amplification kit (Applied Biosystems/Ambion). 750 ng of labeled cRNA was hybridized overnight to Illumina Human HT-12 V4 BeadChip arrays (Illumina), which contained more than 47,000 probes. The arrays were washed, blocked, stained and scanned on an Illumina iScan, as per manufacturer's instructions. GenomeStudio (Illumina) was then used to perform quality control and generate signal intensity values.

South African and UK 2009 sample's RNA was isolated as previously described and hybridized to Illumina Human HT-12 V3 BeadChip arrays (Illumina) (9). Probes were translated from the HT-12 V3 BeadChip arrays to HT-12 V4 BeadChip arrays using GeneSpring GX version 11.5 (Agilent Technologies) and translated to slightly fewer probes in V4.

Raw data were processed using GeneSpring GX version 11.5 (Agilent Technologies), and the following was applied to all analysis. After background subtraction each probe was attributed a flag to denote its signal intensity detection p-value. Flags were used to filter out probe sets that did not result in a ‘present’ call in at least 10% of the samples, where the ‘present’ lower cut off=0.99. Signal values were then set to a threshold level of 1, log 2 transformed, and per-chip normalised using 75th percentile shift algorithm. Next per-gene normalisation was applied by dividing each messenger RNA transcript by the median intensity of the latent TB samples. All statistical analysis was performed after this stage.

All data collected and analyzed in the experiments adhere to the Minimal Information About a Microarray Experiment (MIAME) guidelines.

Statistical Analysis: GeneSpring 11.5 was used to select transcripts that displayed a degree of expression variability. A filter was set to include only transcripts that had at least twofold changes from the median intensity of all latent TB samples and present in at least 10% of the samples. This approach was used to determine all the ‘active TB-transcriptional signatures’. To divide the South Africa 2011 cohort into a training and test set, a computer algorithm was used for randomization (12). For the specific treatment response signature transcripts had to satisfy a threefold expression filter in eight of the 11 training set matched untreated and six month treated samples. Selected transcripts were then filtered by different levels of statistical stringency in GeneSpring 11.5. Non-parametric tests with multiple testing corrections were applied to all analyses (10, 11). The active TB-transcriptional signatures were generated by Mann Whitney unpaired Benjamini Hochberg p<0.01 or Bonferroni p<0.01 (FIG. 2B). The statistical filter used to generate the specific TB treatment response-transcriptional signature was Mann Whitney paired Benjamini Hochberg p<0.05. The 393 and 86 active TB signatures were obtained as described previously (FIG. 8) (9). The transcript lists for each signature were then applied to the cohorts they were derived from and/or to the following cohorts: South Africa 2011 active TB Training and Test Set, UK 2011 Cohort and the three cohorts from an earlier study. Visualization of the data was performed by heatmaps using hierarchical clustering where the correlation distance metric employed for the clustering was Pearson's uncentered with average linkage (13). Heatmaps displayed either hierarchical clustering of both transcripts and samples or hierarchical clustering of transcripts with forced grouping of samples. Visualization of common and different transcripts by Venn diagrams was performed in GeneSpring 11.5. Translation of probes/transcripts between V3 HT12 and V4 HT12 chip was performed using the probe ID and Illumina specific probe_id. Slightly fewer probes were translated from V3 to V4.

Molecular distance to health (MDTH) was determined for each time point, as previously described (14). Temporal Molecular Response was determined per individual, for each transcriptional signature, by calculating the sum of the transcripts that were greater than twofold up or down at a specific time point, e.g. two weeks, compared to the raw pre-treatment intensity values. For intensity values of zero a value of 10 to the power of −20 (10⁻²⁰) was introduced. The calculated number of altered transcripts was then expressed as a percentage of the total number of transcripts in the transcriptional signature. This calculation was then repeated for the rest of the time points. MDTH and temporal molecular response were calculated in Microsoft Excel 2010. GraphPad Prism version 5 for Windows was used to generate graphs, determine linear regression, and determine associated p-values using either Friedman and Dunn's multiple comparison test for MDTH data or ANOVA repeated measures and Tukey's multiple comparison test for temporal molecular response data. Linear mixed models, fixed effects, was performed in SAS/STAT® software (SAS Institute Inc., USA). Statistical tests applied were dependent on the distribution of the data as determined by D'Agostino and Pearson omnibus normality test. Pathway analyses were performed using Ingenuity Pathway Analysis (Ingenuity Systems, Inc., Redwood, Calif.). Canonical pathways analysis identified the most significantly represented pathways in the datasets (Fisher's exact Benjamini Hochberg p<0.05).

Results: Participants Demographics and Characteristics: Participant numbers in the South Africa 2011 cohort are described in FIG. 1; 29 active TB patients were recruited and sampled for transcriptomic analysis; all active TB patients were treated for six months with quadruple antitubercular therapy (rifampin, pyrazinamide, isoniazid and ethambutol) for two months followed by rifampin and isoniazid for four months. Of these, 20 were resampled after two weeks, and after two, six and 12 months after initiation of treatment; blood from 38 latent individuals was sampled as asymptomatic controls. Demographics and clinical characteristics of the South Africa 2011 and UK 2011 cohorts are reported in Tables 2A and 2B. Patient demographics and clinical characteristics of the 2009 South Africa cohort and UK cohorts have been previously described (9). All treated active TB patients included in the study had drug sensitive treatment, took all treatment prescribed, showed successful clinical/radiological response to treatment, did not relapse within one year and were discharged from the program as cured.

A Change in Transcriptional Response is Readily Detectable after Two Weeks of Treatment: To determine whether an active TB transcriptional signature in the blood of the 2011 South Africa cohort was perturbed upon treatment, gene expression profiles of only significantly detectable genes without further filtering (detected p<0.01 from background, 16,856 transcripts), were examined in the 20 active TB patients before, during (two weeks and two months), at the end (six months), and after treatment (12 months). By plotting the expression profiles of the 16856 transcripts along a time scaled x-axis, a marked change was readily observed after two weeks of anti-TB treatment (FIG. 2A).

An active TB 664-transcript signature was derived from differentially expressed genes in the active TB patients compared to the latent TB patients in the Untreated South Africa 2011 cohort (FIG. 2B). First, all transcripts were normalized to the median of the latent TB patients, then only transcripts with an equal or greater than twofold change from the median were selected, before finally applying a stringent statistical filter (Bonferroni; FIG. 2B; 664 transcripts). When these signatures were applied to the Treated South Africa 2011 Cohort, a marked and rapid change in the transcriptional response was observed as early as two weeks, which then continued through two and six months after treatment initiation (FIG. 2B). As previously reported, Ingenuity Pathway Analysis (IPA) of these blood transcriptional signatures demonstrated a highly significant over-representation of Interferon (IFN)-signaling genes including Type I and Type II IFN (FIGS. 2C and D, p<0.001).

The Transcriptional Response Changes Significantly at Two Weeks, Two Months & Six Months after Treatment Initiation: Since the South Africa untreated active TB signatures diminished in response to treatment, the present inventors determined that there was a significant change in the transcriptional signature during treatment. For this determination, the molecular distance to health (MDTH) algorithm was determined as this generates a quantitative score for the degree of transcriptional perturbation in a disease cohort relative to the controls (14). The present inventors recognized that MDTH positively correlates with the severity of active pulmonary TB, as defined by the radiological extent of disease (9). The present inventors found that the median MDTH associated with the 664 South African untreated active TB-transcriptional signature altered significantly at two, six, and twelve months, compared to the median pre-treatment MDTH (FIG. 2E).

To expand on the treatment induced transcriptional response, a metric was developed that allowed us to evaluate each individual's change in gene expression relative to their own expression profile, rather than relative to a control group. This ‘temporal molecular response’ offers a potential advantage in the clinical setting to allow separate assessment of each patient's outcome. For a given signature, the temporal molecular response was determined by measuring the transcriptional perturbation between two time points, and expressing this value as a percentage of the total number of transcripts constituting the signature. The mean temporal molecular response associated with the South Africa untreated active TB 664 transcript signature altered rapidly and significantly as early as two weeks, and continued to alter significantly at two months and six months (FIG. 2F). This finding demonstrates that the transcriptional signature not only changes rapidly and as early as two weeks, but also continues to significantly change between two weeks and two months.

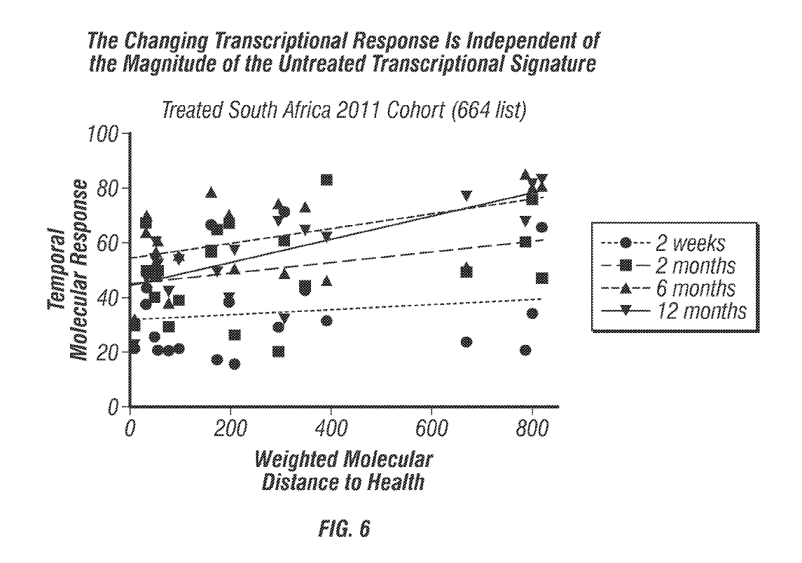

The changing transcriptional response is independent of the magnitude of the untreated transcriptional signature: It may be predicted that individuals with more extensive disease would respond to treatment differently from those with minimal disease. However the magnitude of the patient's temporal molecular response during treatment, at both two weeks and two months, did not correlate with the magnitude of their pre-treatment transcriptional signature, as evidenced by MDTH (r2<0.1, non-significant) (FIG. 6). However, the patient's temporal molecular response after treatment, at six months and 12 months did significantly correlate with their pre-treatment MDTH (r2=0.25, p=0.02 and r2=0.57, p=0.001 respectively) (FIG. 6). This finding, that the magnitude of the pre-treatment signature does not appear to be able to predict the patient's response, further underscores the benefit of the temporal molecular response in offering continuous monitoring throughout treatment.

A specific ‘TB treatment response signature’ significantly diminishes at two weeks, two months and after completion of treatment: The 664-transcript active TB signature significantly and rapidly changed in response to treatment (FIGS. 2B, E and F). This signature was derived by identification of differentially expressed genes between untreated active TB and latent TB patients.

In addition, a transcriptional signature that specifically reflected the response of patients to clinically successful anti-TB treatment (comparing time points 0 and 6 months) was determined. To determine the treatment specific signature, a computer algorithm was used to randomize the South Africa 2011 cohort into two groups of patients (12) (FIG. 1). This allowed us to derive the signature from one group of patients (Active TB Training Set) and then validate findings in another independent group of patients (Active TB Test Set). Upon analysis, 391 transcripts were found to be significantly differentially expressed between the untreated Active TB Training Set samples and their matched six-month treated samples. The 391-transcript treatment specific signature was shown to rapidly and significantly change at two weeks, two and six months after treatment initiation in the Active TB Training set. This was validated in the Active TB Test Set (FIG. 3A-E). In both cohorts the change in the temporal molecular response was significant at two weeks post-treatment (FIGS. 3B and D). Analysis of the 391 transcripts by IPA indicated the most significantly represented pathways were related to the innate immune pathways, encompassing genes related to complement, Toll-like receptors, a NOD like receptor gene and interleukin-1B, which were all significantly altered with treatment (FIG. 3E). Of the 391-transcript treatment signature genes, 68% were also present in the 664-transcript active TB signature (FIG. 3F).

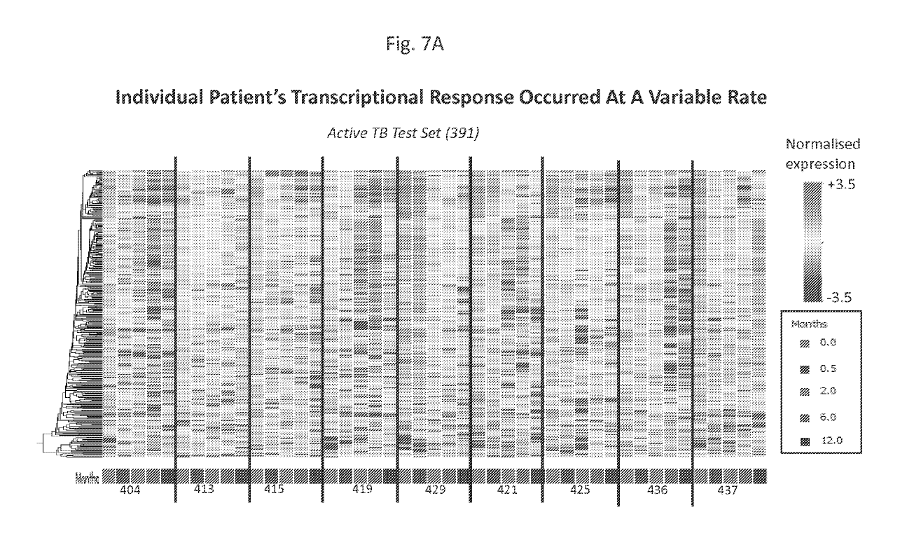

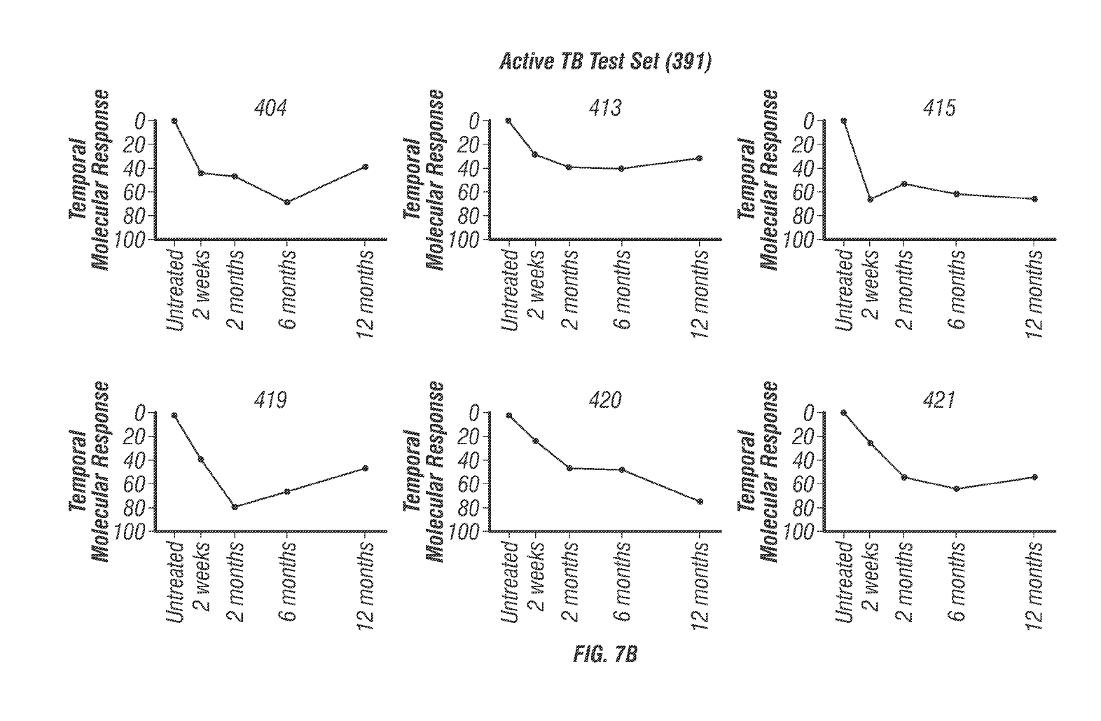

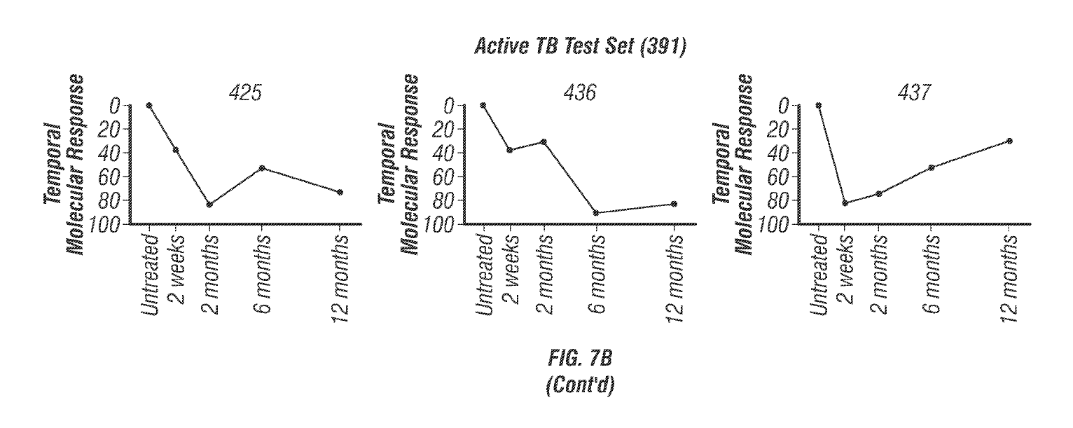

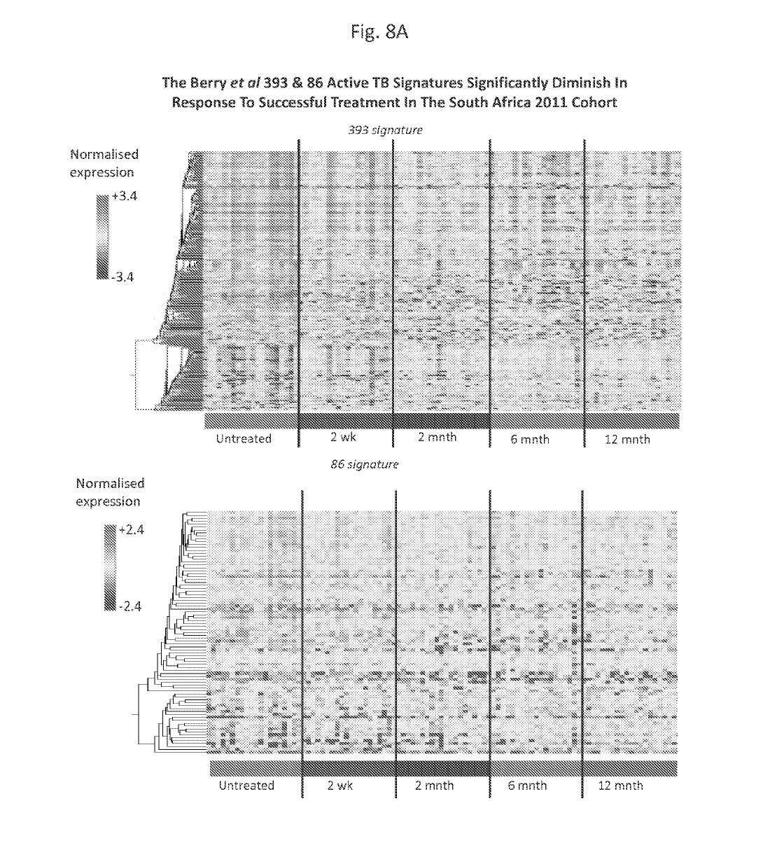

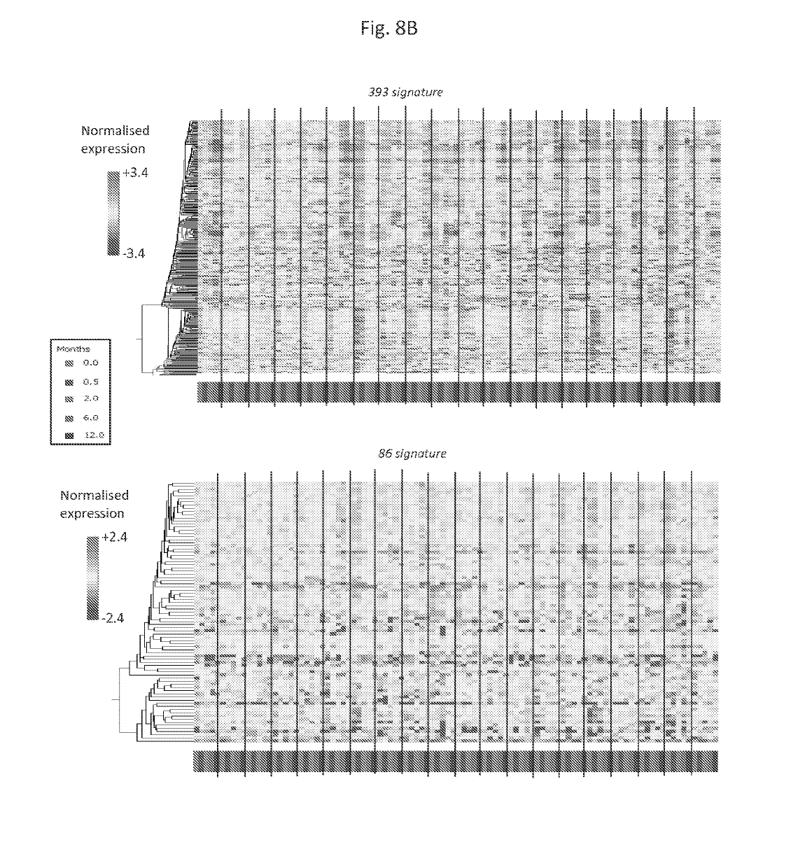

Measuring an individual patient's transcriptional response to anti-TB treatment: Each patient's discrete treatment response is shown in the heatmaps and by graphical form using the temporal molecular response in FIG. 4 and FIG. 7. All 20 patients in the active TB treated cohort had a rapid and early positive temporal response after two weeks of treatment. Interestingly, not all the individual transcriptional responses were identical (FIG. 4A, FIG. 7A) as demonstrated by the quantitative scoring provided by the temporal molecular responses (FIG. 4B, FIG. 7B).

By both the MDTH and temporal molecular response, it was observed that none of the transcriptional signatures revealed significant differences between two months and six months post treatment initiation (FIGS. 2E and F; FIGS. 3B and D; and FIG. 8C). This could suggest that the transcriptional response reaches a plateau at two months and would therefore be no different from latent TB patients. Therefore, each of the time points was compared: two, six, and 12 months to the latent TB expression profiles. It was determined that 151 transcripts were differentially expressed between two months and latent TB (Mann Whitney paired Benjamini Hochberg p<0.01). However no genes were significantly differentially expressed between six & 12 months, six months & latent TB, and 12 months & latent TB (Mann Whitney paired Benjamini Hochberg p>0.01).

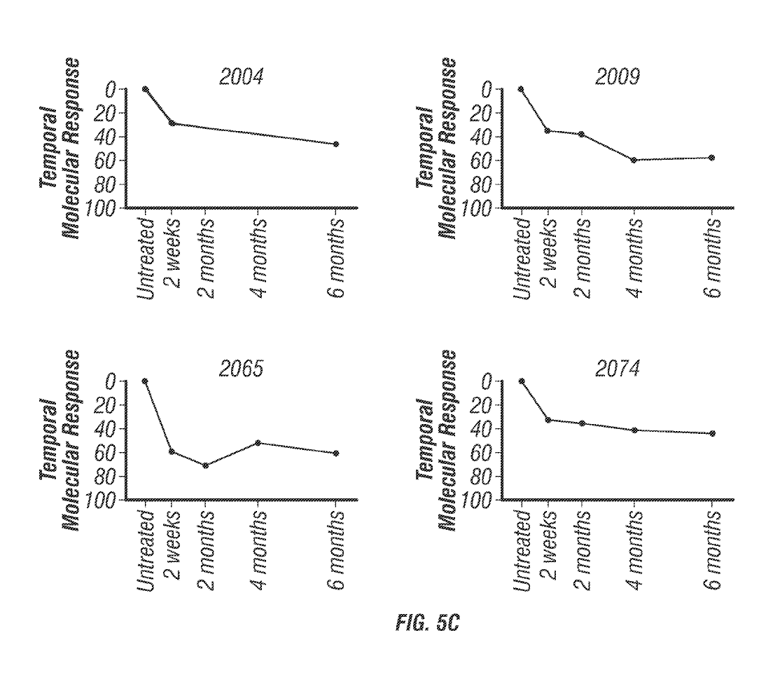

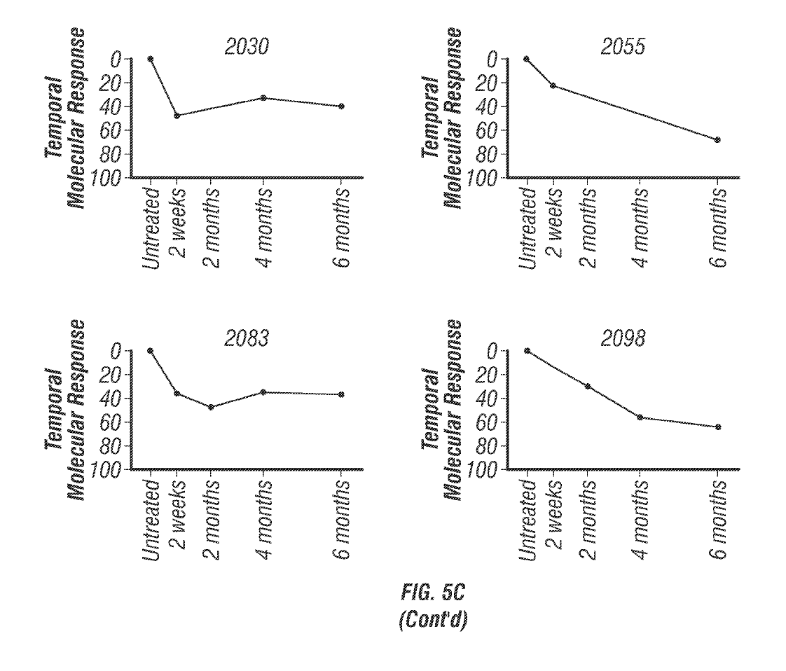

Validation of early anti-TB treatment blood transcriptional response: To determine whether the significant change in the 391-transcript treatment specific signature that had been demonstrated in a South Africa cohort was also generalizable to patients in an intermediate burden setting, the signature was tested in UK. As observed in the South Africa cohort the signature was rapidly and significantly diminished at two weeks onwards post commencing treatment (FIGS. 5A and B). The changes in the blood transcriptional response could be clearly quantified in individual patients as shown by the temporal molecular response (FIG. 5C). The significant transcriptional blood change correlated with successful treatment of patients as assessed after six months by radiographic and clinical parameters.

The significant change in transcriptional response at two weeks occurs when applying active TB signatures derived from other cohorts: thus far it was demonstrated that the 664-transcript active TB signature (FIG. 2), and the treatment specific 391-transcript signature (FIG. 3) diminished rapidly and significantly with treatment at two weeks. Then it was sought to determine if active TB transcriptional signatures derived from other cohorts would also diminish in response to treatment. Therefore active TB transcriptional signatures derived from cohorts used in the earlier Berry et al. (2010) study (9) were applied to all the time points of the Treated South Africa 2011 samples.

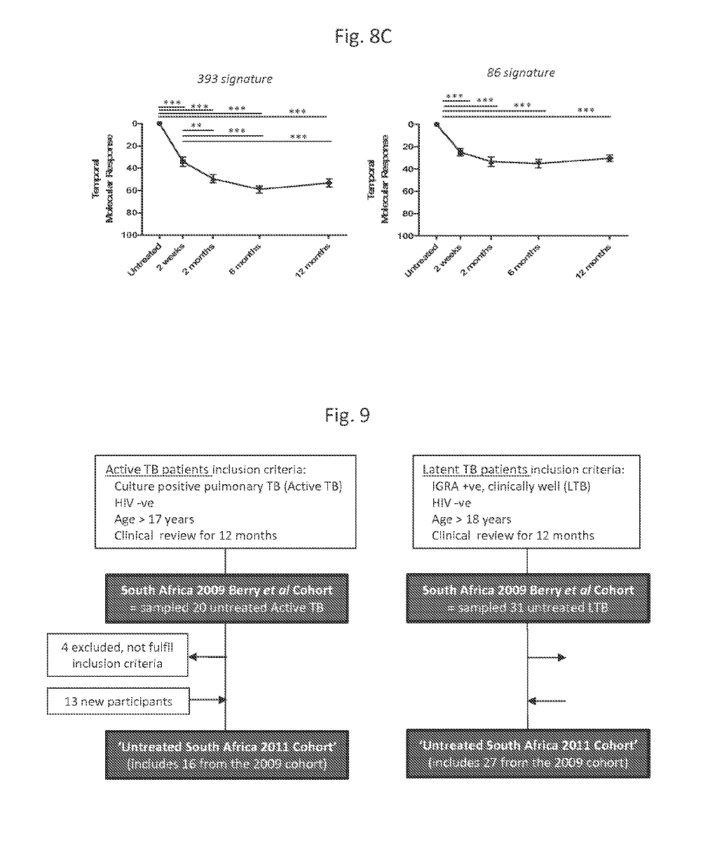

The established 393- and 86-transcript active TB signatures from the earlier Berry et al. (2010) study (9) obtained by comparing active TB patients to latent TB patients and healthy controls were applied first. The present inventors demonstrate that the 393 and 86-transcript active TB signatures significantly and rapidly diminished in the South Africa 2011 cohort, and this occurred as early again as two weeks (FIG. 8A-C). Additionally active TB signatures from the Berry et al (2010) cohorts were derived, only comparing active TB patients to latent TB patients (removing healthy controls), using the same analysis approach as was used to determine the 664-active TB signature in the Untreated South Africa 2011 cohort (FIG. 2A-C and FIG. 2E-F). The active TB transcriptional signatures thus obtained from the different cohorts (named 2009 UK Training set, 2009 UK Test set and 2009 South Africa set, FIG. 9) showed significant changes in the transcriptional response in the Treated South Africa 2011 Cohort at two weeks, two months and at the end of treatment (FIG. 10A-C).

A core set of genes (344 transcripts) were found to be overlapping between these 2009 derived active TB transcriptional signatures (FIG. 10D). The overlapping genes had many genes in common with both the South Africa 2011 derived active TB 664-transcript signature and the 2011 treatment specific 391-transcript signature (FIG. 10E).

The whole blood active TB-transcriptional signatures derived from a South African cohort, dominated by IFN signaling and innate immune response genes, showed a readily detectable change in response to clinically successful anti-TB treatment. Importantly the treatment-associated changes in the active TB-transcriptional signature and a specific TB treatment response-transcriptional signature were rapid and highly significant as early as two weeks after the initiation of therapy. The transcriptional response to treatment could be individually measured in each patient and was independent of the magnitude of their pre-treatment transcriptional signature. The significant and early change in the treatment specific transcriptional signature was then validated in a UK cohort. These findings demonstrate that blood transcriptional signatures can be pragmatic as early surrogate markers of a successful treatment response, and can be used as biomarkers in both the clinical setting and in drug development. In certain embodiments, the method is useful for improving stratification and monitoring of clinical treatment of active TB patients, testing novel therapies in to enhance efficacy in treatment of drug-sensitive Mtb infection in clinical trials, and in the testing of novel drugs for use in the potential treatment of MDR- and XDR-TB.

An active TB transcriptional signature, originally derived from a UK cohort, which distinguished active TB patients from patients with other inflammatory and infectious diseases and which correlated with the radiographic extent of disease, was demonstrated (9). The transcriptional response in the high-burden TB country of South Africa was evaluated before, during, and after anti-TB treatment. The study of TB patients in both the UK and South Africa provided gene expression profiles across diverse host populations, exposed to different local environments and likely different Mtb strains. The South African cohort contained participants from Khayelitsha, a large peri-urban African township in Cape Town, where 1.5% of the population develops active TB annually. It was found that South Africa active TB patients (all HIV uninfected) had more differentially expressed genes that those from the UK. This may be explained by a higher incidence of co-infection with other micro-organisms and viruses, besides HIV, or a higher burden of Mtb infection due to delayed diagnosis relative to countries like the UK, present in South Africa. For example, although the exact helminth prevalence in adults is unknown, data from surveys suggest between 70-100% of children are infected (15). Although the number of genes differed between the South Africa and UK cohorts, the most significantly represented pathways, IFN signaling and innate immune response pathways, were the same. Notably many of the genes contained within the innate immune response pathways in all the cohorts, were also interferon inducible genes (Table 1), including the complement genes, Toll-like receptor genes, and familiar IFN inducible genes such as CXCL10 and OAS genes (16-20).

Studies in TB. Maertzdorf et al examined whole blood gene expression of active TB and latent TB patients in cohorts from both South Africa and The Gambia (21, 22). Although some IFN inducible and innate response genes were significantly over expressed in the active TB patients, different microarray chips and analysis strategy were employed.

While it is widely appreciated that the diagnosis of TB has many difficulties, the present inventors recognize that TB treatment monitoring is a difficult challenge in trying to eradicate Mtb infection. So much so that in April 2010 the Centers for Disease Control and National Institutes of Health brought together experts in the field and research scientists with the sole purpose of addressing this problem (23). The consequences of poor treatment monitoring, and therefore impending inadequate treatment, includes worsening of a patient's disease, increasing potential for disease spread and most worryingly an escalation in drug resistant mycobacteria. Currently the two-month sputum culture conversion rate, used to measure anti-TB treatment response, is the only biomarker of successful TB treatment (6). However sputum culture conversion is a time consuming test, since it takes several weeks to grow the bacilli and results can be compromised by contamination. In fact, often the patients who have improved are unable to expectorate sputum at two months but then are wrongly labeled as having a negative result (24). Furthermore although sputum conversion has efficacy as a surrogate end point of treatment response in clinical trials evaluating new drugs, a systematic review and meta-analysis to assess its accuracy to predict an individual's treatment failure revealed low sensitivity and modest specificity (7, 25). While other biomarkers have also been trialed, including C-reactive protein, IFN-γ and neopterin, all have shown poor sensitivity and specificity (26). Chest X-rays are commonly used in the clinical setting as a marker of treatment response but they generally improve more slowly than the clinical response and lack specificity as interpretation can be confounded by previous lung damage (24). Moreover, interpretation of chest X-ray changes in response to treatment has not yet been standardised, and the facilities are not always available in developing countries (8). Therefore there is clearly a need for early and easily detectable biomarkers for treatment monitoring, capable of detecting drug resistance or poor treatment adherence and available for patients unable to produce sputum. In addition, such blood biomarkers of early anti-TB treatment response would be vital in clinical trials to aid the evaluation and development of more effective new and shorter treatment regimens.

In UK patients, active TB signatures, 393 and 86 transcripts, diminished at two months of treatment (9). The present inventors now show a significant blood transcriptional response to treatment occurs rapidly and as early as two weeks (FIGS. 2A, 2B, 2E, 2F, 3A-D, 4A-B, 5A-C, 7A-B. 8A-C, 10A-C) or sooner. This significant change in transcriptional response continued between two weeks and two months and again between two months and six months. It was demonstrated that this significant change occurred in all active TB-transcriptional signatures derived from both South Africa and UK cohorts. The use of this approach as an early biomarker correlating with treatment response strengthens its use as an adjunct diagnostic tool as well as an early treatment biomarker. A significant response in the treatment specific-transcriptional signature, derived from a clinically successfully treated active TB cohort, and validated in two other treated active TB cohorts, was demonstrated (FIGS. 3C-D and 5A-C). This treatment specific transcriptional signature also had many genes in common with the active TB transcriptional signatures. Therefore, this study will help guide future exploration for a highly specific subset of genes that explicitly correlates with a patient's mycobacterial response to anti-TB treatment, therefore acting as a surrogate marker of treatment failure or success.

Although no other studies have looked directly at the transcriptional response to TB treatment, two other studies have observed some transcriptional changes, but only measured at two months or after treatment completion. Mistry et al. studied whole blood gene expression in patients from South Africa, comparing patients with active TB, recurrent reactivation of TB, cured TB and latent TB (28). These study methods were different from the methods employed herein because Mistry et al. did not measure the transcriptional profile during treatment but only after completion of treatment. Mistry et al. showed that those who were cured from TB displayed similar expression profiles to those with latent infection. There was a small but non-significant increase in gene expression six months after stopping the anti-TB treatment in a small number of active TB patients (e.g., FIG. 4B). This may be explained by the continuous high exposure to infections in South Africa, as anti-TB treatment consists of antibiotics capable of treating a broad-spectrum of bacteria. Joosten et al. showed in a small cohort from The Gambia that their active TB gene set, distinguishing active from latent TB, also diminished at two months of treatment (29). Embodiments of the present invention, however, demonstrate the accomplishment of a novel, inexpensive, fast automated molecular method to measure blood gene expression profiles. Techniques such as this can be applied to early treatment blood transcriptomic of TB treatment response. Therefore early treatment blood transcriptome analysis of TB treatment response at two weeks (or sooner) has great potential for development as a pragmatic blood biomarker for clinical use.

A further problem in the management of TB is the extended length of treatment, requiring a minimum of six months, which has a negative impact on patient adherence and treatment completion. Therefore the ability to stratify patients into groups that may require shorter lengths of treatment, particularly in resource limited settings, could be of value in improving patient compliance and reducing treatment related side effects. It is shown herein that transcriptional response of some patients appeared to plateau before six months (FIGS. 4B, 5C and 7B), suggesting a tailored treatment response for individual patients may be possible, and that blood transcriptional signatures could help with this stratification.

### Example 2

This example provides further advances by the present inventors in methods for monitoring changes in blood transcriptional signatures in response to antituberculosis treatment and details use of these changes as early biomarkers of a successful response. In particular, significant changes in the transcriptional signatures measured by blood tests were readily detectable just two weeks after treatment initiation, and transcriptional response to treatment is shown as being readily measured in individual patients. These findings further support that blood transcriptional signatures are useful as early surrogate biomarkers of successful treatment response. Unlike Example 1, use of a 320 gene (Table 12) transcriptional signature is prominently disclosed in Example 2.

More specifically, blood transcriptional profiles of untreated active tuberculosis patients in South Africa were analysed before, during (two weeks and two months), at the end of (six months) and after (12 months) antituberculosis treatment, and compared to individuals with latent tuberculosis. An active-tuberculosis transcriptional signature and a specific treatment-response transcriptional signature were derived. The specific treatment response transcriptional signature was tested in two independent cohorts. Two quantitative scoring algorithms were applied to measure the changes in the transcriptional response. The most significantly represented pathways were again determined using Ingenuity Pathway Analysis. An active tuberculosis 664-transcript signature and a treatment specific 320-transcript signature significantly diminished after two weeks of treatment in all cohorts, and these continued to diminish until six months. The transcriptional response to treatment could be individually measured in each patient.

Active pulmonary TB diagnosis requires culture of Mtb, which may take up to six weeks [3]. Although the World Health Organization (WHO) endorsed GeneXpert MTB/RIF automated molecular test for Mtb results in rapid diagnosis [4], this test still requires sputum which may be difficult to obtain. Difficulties in obtaining sputum lead to approximately 30% of patients in the USA and 50% of South African patients to be treated empirically [2], [5]. After diagnosis there are no available early biomarkers correlating with treatment success, resulting in significant delay in assessing treatment response. Currently conversion to negative culture after two months of treatment is the only accepted biomarker [6]. However a systematic review and meta-analysis of sputum conversion revealed low sensitivity and modest specificity for the prediction of treatment failure [7]. Chest X-rays are commonly used to assess response but are not universally available and assessment is difficult to standardise [8]. This lack of effective treatment monitoring can lead to the development and spread of multidrug resistant (MDR) and extensively drug resistant (XDR) TB, which are mainly caused by non-adherence or inappropriate drug regimens, with a detrimental impact on global TB control.

To date transcriptional profiling has been used successfully in cancer classification, to identify prognostic biomarkers [9], and to distinguish between inflammatory and infectious diseases [10]. Moreover, a whole blood transcriptional signature may be used to distinguish active TB from latent TB and other diseases, and it is correlated with radiographic extent of disease [11]. This active TB blood signature diminished in seven patients after two months of successful treatment and reverted to that of healthy individuals after completing treatment [11]. Earlier blood biomarkers correlating with treatment response would improve monitoring of individual patient treatment responses without the need for sputum production, which may permit stratification of patients requiring differing treatment regimens. Additionally, early biomarkers may aid in anti-TB drug development.

The study detailed in this example was designed to establish if early changes in blood transcriptional responses can be observed during standard anti-TB treatment. It adds to previous studies in part by examining the transcriptional treatment response directly in a larger cohort from a high-burden TB country, South Africa [2].

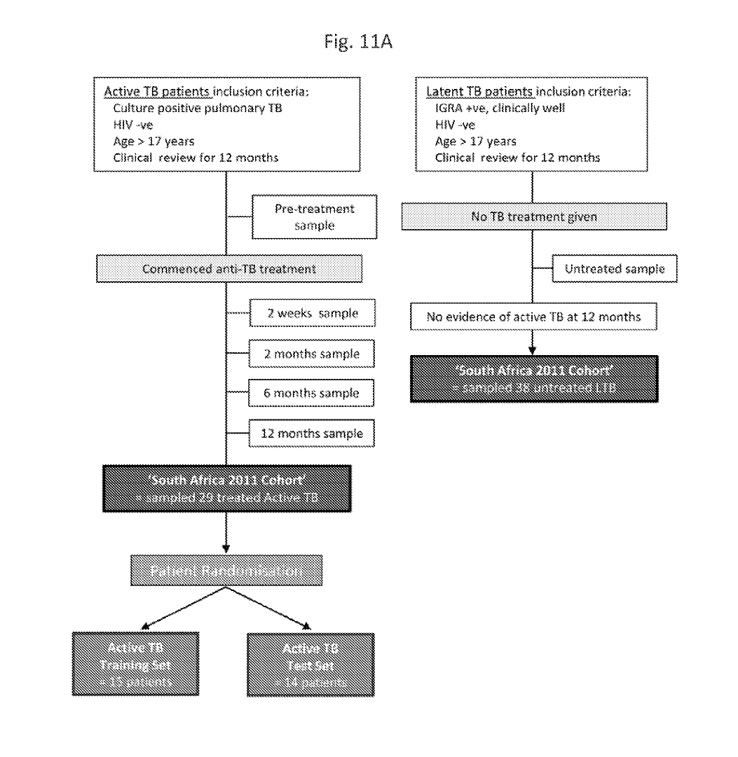

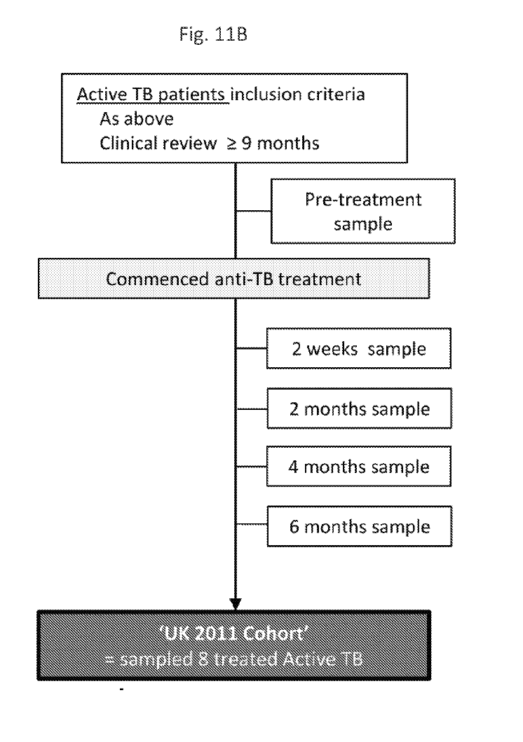

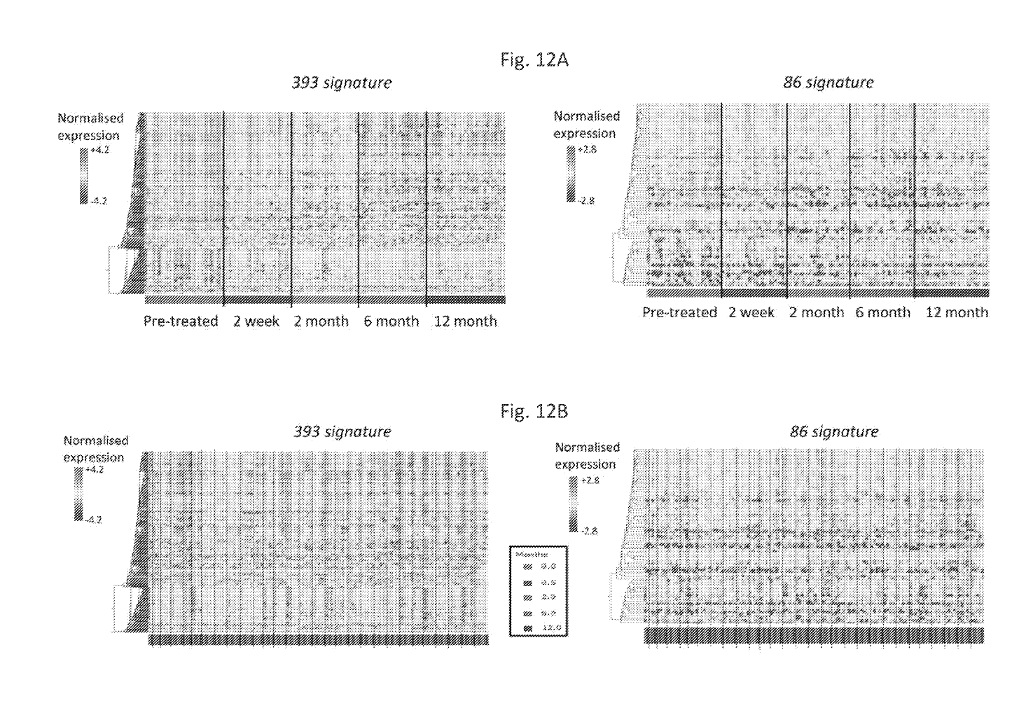

Materials and Methods. All participants in South Africa were recruited from the Ubuntu TB/HIV clinic in Khayelitsha, a large peri-urban African township in Cape Town which has over 1000 TB notifications annually. During the period May 2008-August 2010 whole blood was collected from adult patients (age>17 years) with drug sensitive Mtb culture proven active pulmonary TB (FIG. 11A). Due to the population's high Mtb exposure, controls were considered as asymptomatic individuals with previous exposure to Mtb (latent TB patients); exposure was evidenced by a positive QuantiFERON-TB Gold In-Tube (QFT) (Cellestis). Participants with latent TB were recruited from individuals self-referring to the voluntary testing clinic. All participants had negative HIV status.

The UK 2011 Active TB Validation Cohort were all Mtb culture proven adults (>17 years) recruited between August 2009-November 2011 from the Royal Free Hospital, London (FIG. 11B). All participants in an earlier 2009 study were selected as previously described [11]. Clinical and demographic data was recorded for all participants and stored in a database.

Ethics Statement. This study was approved by the University of Cape Town Faculty of Health Sciences Human Research Ethics Committee, Cape Town, South Africa (FHS HREC 012/2007), and the Central London 3 Research Ethics Committee (09/H0716/41). All participants gave informed written consent.

Follow Up Period. All 29 treated 2011 South Africa active TB patients completed a full 6 months of treatment. Patients were sampled for venous blood at time points: pre-treatment (29/29 patients), 2 weeks (25/29 patients), 2 months (24/29 patients), 6 months (25/29 patients) and 12 months (29/29 patients) after initiation of treatment (FIG. 11A). Patient's response to anti-TB treatment was assessed clinically during the 12 month period. All patients were discharged from the program as cured.

Eight treated 2011 UK Active TB patients completed a full six months of treatment, one patient completed nine months of treatment due to radiographic uncertainty of treatment success. Each patient was sampled for venous blood at two weeks, two months, four months and six months after initiation of treatment (FIG. 11B). Four patients had a sample at every time point, three patients had samples at two, four and six months, and four patients had samples at two weeks and six months. As part of their routine medical care all patients had chest X-rays at the beginning and end of their treatment.

IFNγ, Release Assay Testing. The QFT Assay (Cellestis) was performed according to the manufacturer's instructions.

Gene Expression Profiling. 3 ml of whole blood were collected into Tempus tubes (Applied Biosystems/Ambion) by standard phlebotomy, vigorously mixed immediately after collection, and stored between −20 and −80° C. before RNA extraction. South Africa and UK 2011 sample's RNA was isolated using 1.5 ml whole blood and the MagMAX-96 Blood RNA Isolation Kit (Applied Biosystems/Ambion) according to the manufacturer's instructions. 250 μg of isolated total RNA was globin reduced using the GLOBINclear 96-well format kit (Applied Biosystems/Ambion) according to the manufacturer's instructions. Total and globin-reduced RNA integrity was assessed using an Agilent 2100 Bioanalyzer (Agilent Technologies). RNA yield was assessed using a NanoDrop800 spectrophotometer (NanoDrop Products, Thermo Fisher Scientific). Biotinylated, amplified antisense complementary RNA (cRNA) targets were then prepared from 200-250 ng of the globin-reduced RNA using the Illumina CustomPrep RNA amplification kit (Applied Biosystems/Ambion). 750 ng of labeled cRNA was hybridized overnight to Illumina Human HT-12 V4 BeadChip arrays (Illumina), which contained more than 47,000 probes. The arrays were washed, blocked, stained and scanned on an Illumina iScan, as per manufacturer's instructions. GenomeStudio (Illumina) was then used to perform quality control and generate signal intensity values.

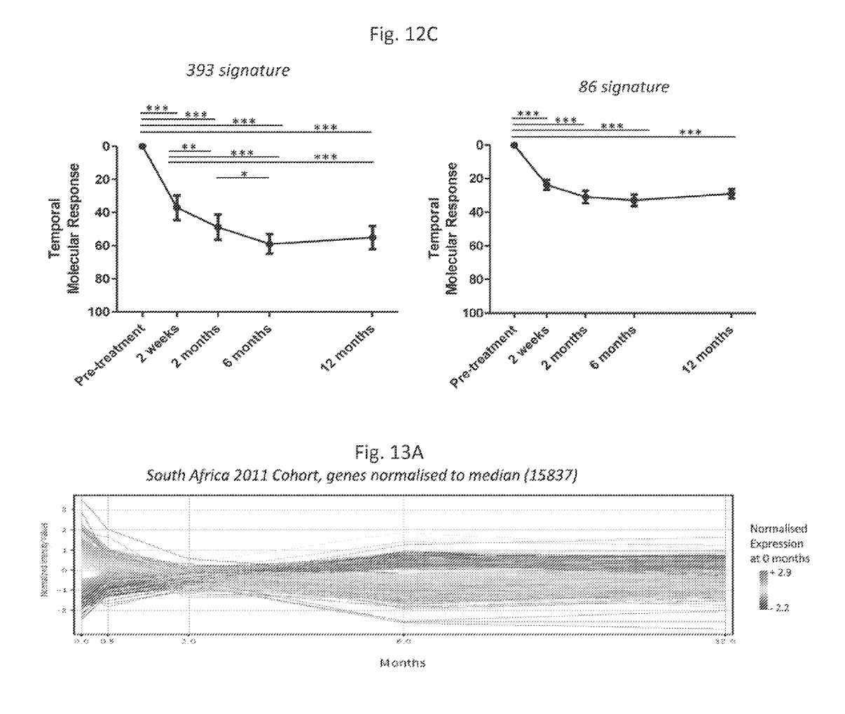

The 393- and 86-transcript signatures were translated from the HT-12 V3 BeadChip arrays to HT-12 V4 BeadChip arrays using GeneSpring GX version 11.5 (Agilent Technologies) and translated to slightly fewer probes in V4 (FIG. 12A-12C).

Raw data were processed using GeneSpring GX version 11.5 (Agilent Technologies) and the following was applied to all analyses. After background subtraction each probe was attributed a flag to denote its signal intensity detection p-value. Flags were used to filter out probe sets that did not result in a ‘present’ call in at least 10% of the samples, where the ‘present’ lower cut off=0.99. Signal values were then set to a threshold level of 1, log 2 transformed, and per-chip normalised using 75^{th} percentile shift algorithm. Next per-gene normalisation was applied by dividing each messenger RNA transcript by the median intensity of the latent TB samples. All statistical analysis was performed after this stage.

The raw and normalised microarray data has been deposited with the GEO (GSE40553). All data collected and analysed in the experiments adhere to the Minimal Information About a Microarray Experiment (MIAME) guidelines.

Data Analysis. GeneSpring 11.5 was used to select transcripts that displayed a degree of expression variability. A filter was set to include only transcripts that had at least twofold changes from the median and present in at least 10% of the samples. To divide the South Africa 2011 cohort into a training and test set, a computer algorithm was used for randomisation [12]. For the specific treatment response signature transcripts had to satisfy a threefold expression filter in 12 of the 15 training set matched untreated and six month treated samples.

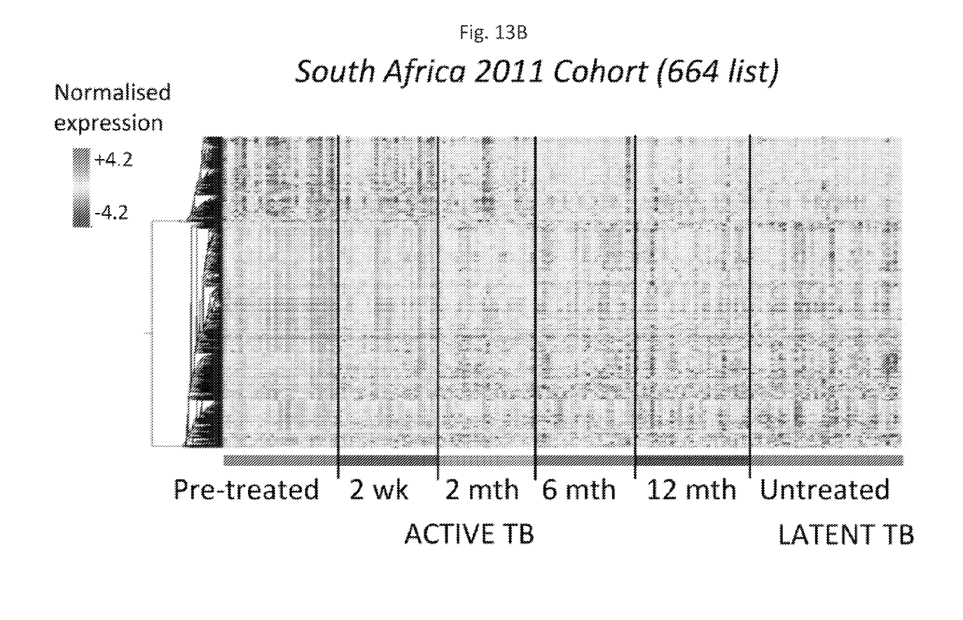

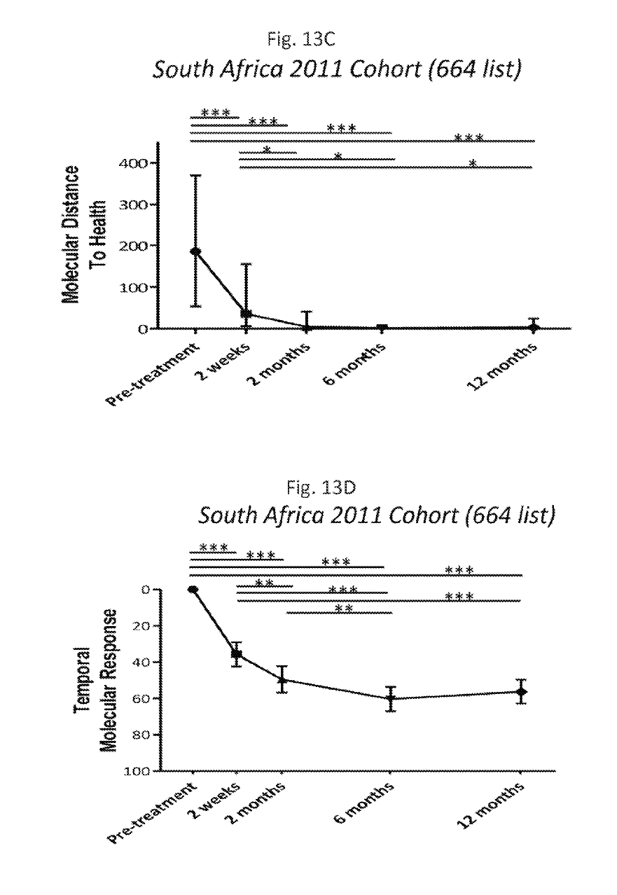

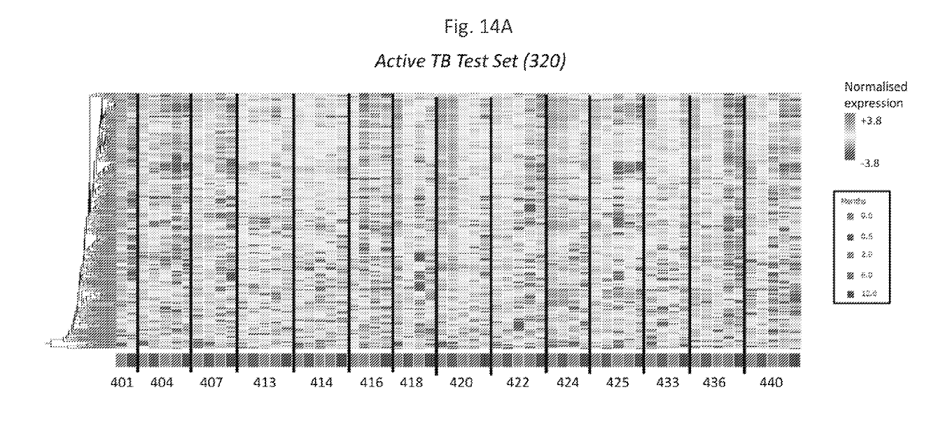

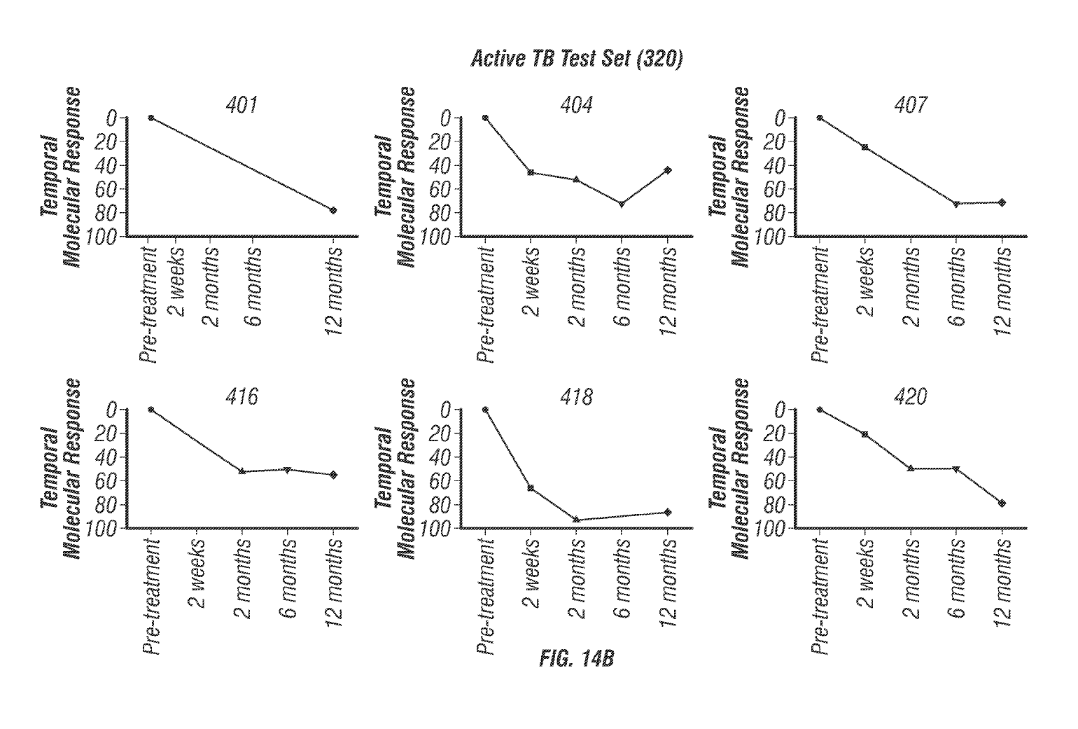

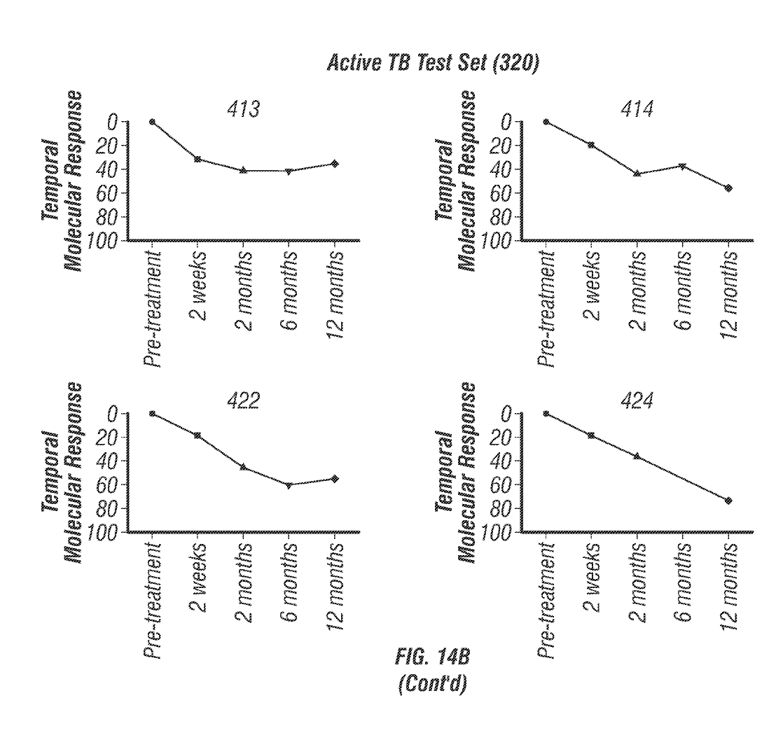

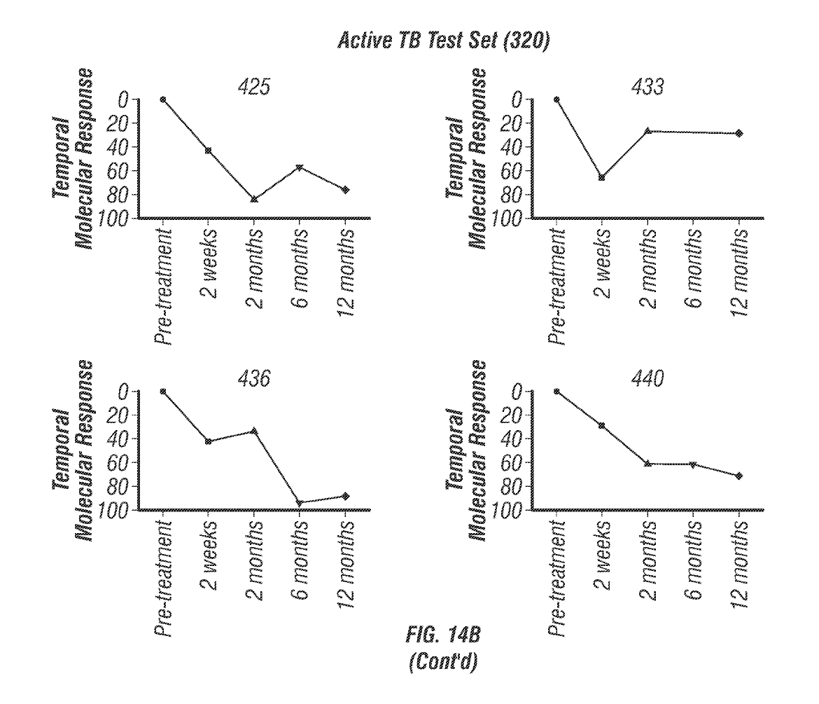

Selected transcripts were then filtered by different levels of statistical stringency in GeneSpring 11.5. Non-parametric tests with multiple testing corrections were applied to all analyses [13], [14]. The active TB-transcriptional signatures was generated by Mann Whitney unpaired Bonferroni p<0.01. FIG. 13A. The statistical filter used to generate the specific TB treatment response-transcriptional signature was Mann Whitney paired Benjamini Hochberg p<0.01. The 393 and 86 active TB signatures were obtained as described previously [11] (see also FIG. 14A-14B). Visualisation of the data was performed by heatmaps using hierarchical clustering where the correlation distance metric employed for the clustering was Pearson's uncentered with average linkage [15]. Heatmaps displayed either hierarchical clustering of both transcripts and samples or hierarchical clustering of transcripts with forced grouping of samples. Visualisation of common and different transcripts by Venn diagrams was performed in GeneSpring 11.5.

Molecular distance to health (MDTH) was determined for each time point, as previously described [16]. The temporal molecular response was calculated for a particular gene list for each individual patient. The raw intensity transcript values in the gene list were consecutively compared at each time point to the baseline (pre-treatment). The numbers of transcripts that were at least two-fold up or two-fold down from the baseline were added together for each time point. This sum was then divided by the total number of transcripts in the gene list to calculate a percentage score for each time point. This generated a percentage score of change at each time point compared to the baseline, where the baseline always remains zero (no change from itself). To allow for two-fold changes from zero any baseline raw transcript intensity values of zero were converted to 10⁻²⁰ (ten raised to the power of minus twenty). MDTH and temporal molecular response were calculated in Microsoft Excel 2010. GraphPad Prism version 5 for Windows was used to generate graphs and determine simple linear regression. Linear mixed models, fixed effects, were used to determine p-values associated with MDTH and temporal molecular response graphs, using SAS/STAT® software (SAS Institute Inc., USA). Pathway analyses were performed using Ingenuity Pathway Analysis (Ingenuity Systems, Inc., Redwood, Calif.). Canonical pathways analysis identified the most significantly represented pathways in the datasets (Fisher's exact Benjamini Hochberg p<0.05).

Results. Participants Demographics and Characteristics. Participant numbers in the 2011 cohorts are described in FIG. 11A-11B; 29 South African and 8 UK active TB patients were recruited and sampled for transcriptomic analysis. All treated active TB patients had fully sensitive Mtb, took all treatment prescribed, showed successful clinical/radiological response to standard therapy (rifampin, pyrazinamide, isoniazid and ethambutol for two months followed by rifampin and isoniazid for four months), did not relapse within one year and were discharged from the program as cured. The 29 South African patients were sampled at: pre-treatment (29/29 patients), two weeks (25/29 patients), two months (24/29 patients), six months (25/29 patients) and 12 months (29/29 patients) after initiation of treatment. Thirty-eight South African latent individuals were sampled as asymptomatic controls. Only five latent individuals were aware of prolonged contact with another individual with active TB. Participant characteristics are reported in Table 13A and Table 13B.

A Change in Transcriptional Response is Readily Detectable after Two Weeks of Treatment. To determine whether an active TB blood transcriptional signature was perturbed upon treatment, gene expression profiles of significantly detectable genes without further filtering (detected p<0.01 from background, 15,837 transcripts) were examined in the 29 active TB patients before, during (two weeks and two months), at the end of (six months), and after treatment (12 months). By plotting the expression profiles of the 15,837 transcripts along a time scaled x-axis, a marked change was readily observed after two weeks of anti-TB treatment (FIG. 13A).

Next an active TB 664-transcript signature (as in Table 8; see also Table S2 at at doi:10.1371/journal.pone.0046191.g001 by Bloom et al. 2012) was derived from differentially expressed genes in the pre-treatment active TB patients compared to the latent TB patients in the South Africa 2011 cohort. First, all transcripts were normalised to the median of the latent TB patients, then only transcripts with ≧twofold change from the median were selected, before applying a statistical filter. When this signature was applied to the South Africa 2011 Cohort, during and after treatment, a marked and rapid change in the transcriptional response was observed as early as two weeks, which then continued through two and six months, after treatment initiation (FIG. 13B). In agreement with the previous study, Ingenuity Pathway Analysis (IPA) of the active TB 664-transcript signature demonstrated a highly significant over-representation of Interferon (IFN)-signaling genes including Type I and Type II IFN (FIG. 13C-D), p<0.001, Table 1; See also Table 1 at doi:10.1371/journal.pone.0046191.t001 (Bloom et al. 2012).

The Transcriptional Response Changes Significantly at Two Weeks after Treatment Initiation. Since it was observed that the South Africa active TB 664-transcript signature diminished in response to treatment, determining if this was a statistically significant change was desirable. To assess this, the previously described weighted molecular distance to health (MDTH) algorithm was employed as this generates a quantitative score for the degree of transcriptional perturbation in a disease cohort relative to the controls [16]. Moreover, as already has been demonstrated, MDTH positively correlates with the severity of active pulmonary TB, as defined by the radiological extent of disease [11]. The median MDTH of the South African untreated active TB 664-transcript signature was found to have decreased significantly at two weeks onwards, compared to the median pre-treatment MDTH (FIG. 13E).

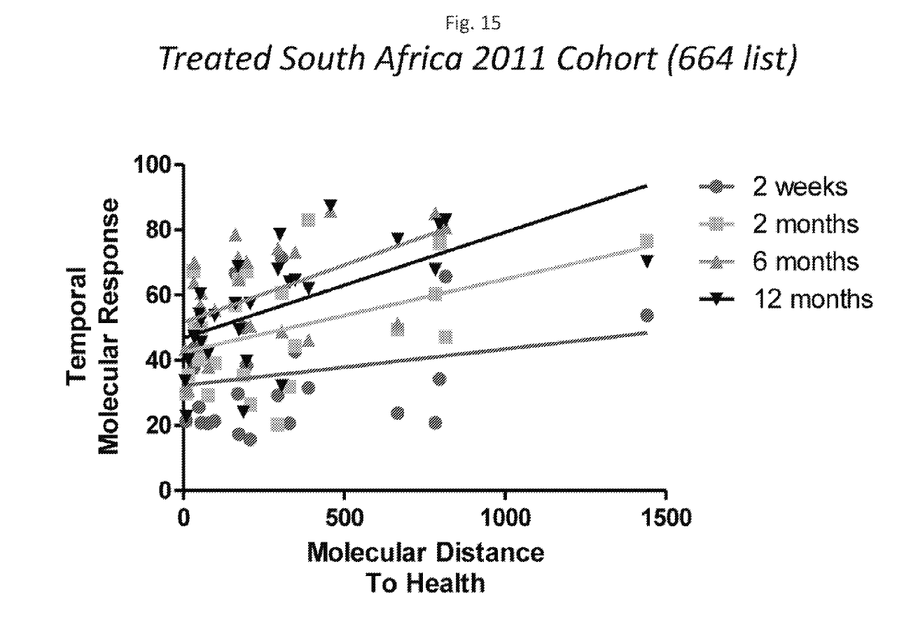

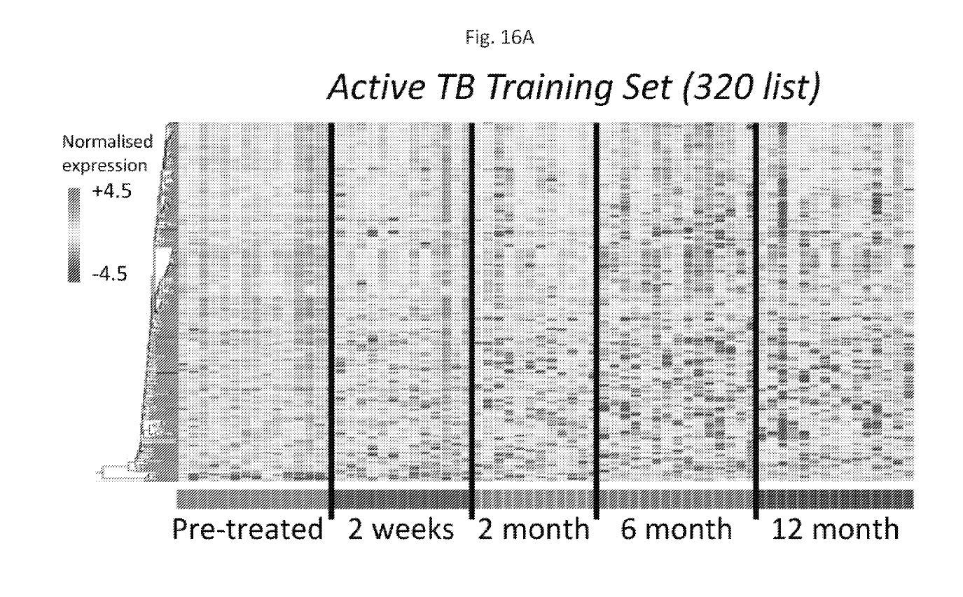

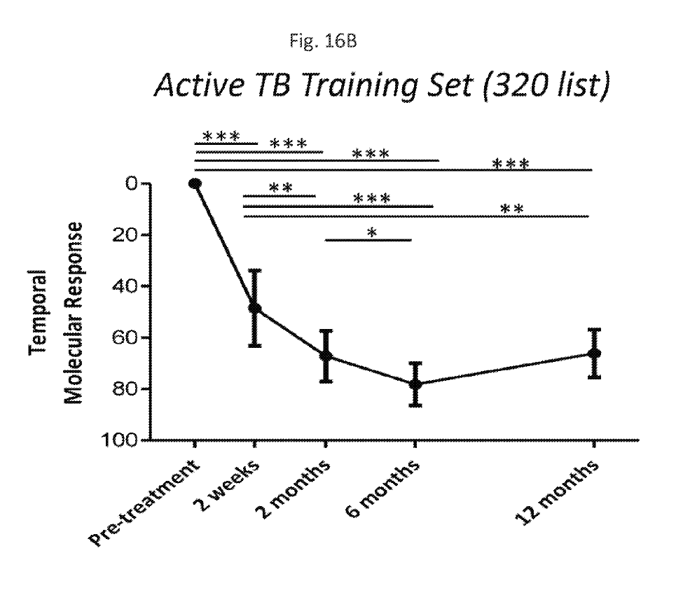

The present inventors then developed a novel metric that provides a quantitative measure of an individual's temporal change in gene expression. This ‘temporal molecular response’ offers a potential advantage in the clinical setting, allowing assessment of each patient's expression change without reference to a control group. For a given signature the temporal molecular response was determined by measuring the transcriptional perturbation between two time points, and expressing this value as a percentage of the total number of transcripts constituting the signature. The mean temporal molecular response calculated for the active TB 664-transcript signature revealed a statistically significant change in the transcriptional response at two weeks after treatment initiation (FIG. 13F). This continued to change between two weeks and two months, and between two weeks and six months, after treatment initiation (FIG. 13F). The magnitude of the patient's temporal molecular response during treatment (at two weeks and two months) did not correlate with the magnitude of their untreated transcriptional signature, as measured by MDTH (p<0.01) (FIG. 15). This suggests a patient's untreated transcriptional signature is not predictive of the patient's treatment response.

As a result, this active TB 664-transcript signature (derived from untreated active and latent TB patients) significantly and rapidly changed after two weeks of initiating treatment (FIGS. 13B, 13E, and 13F).

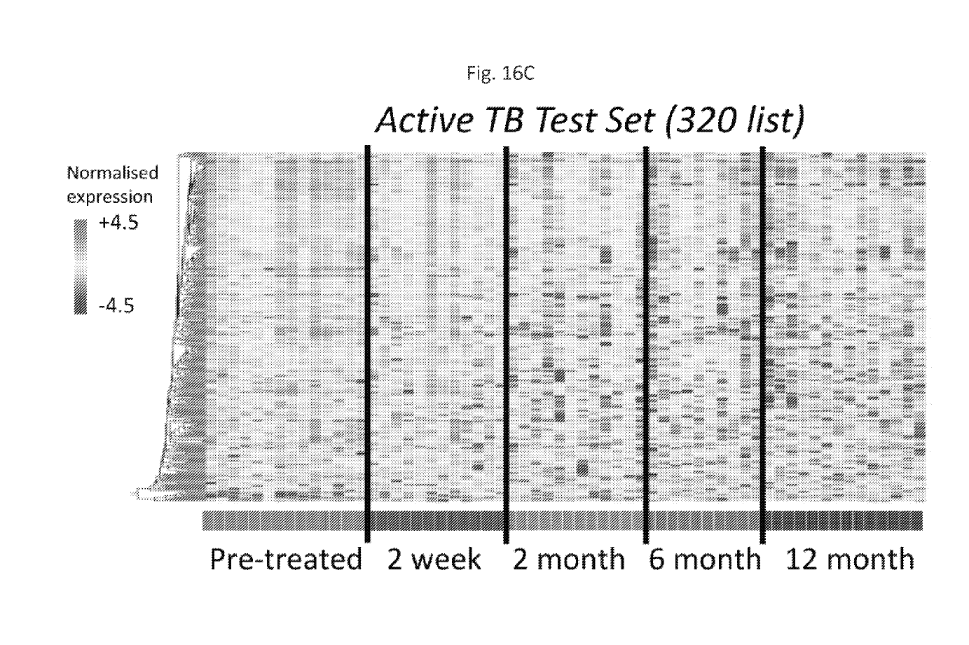

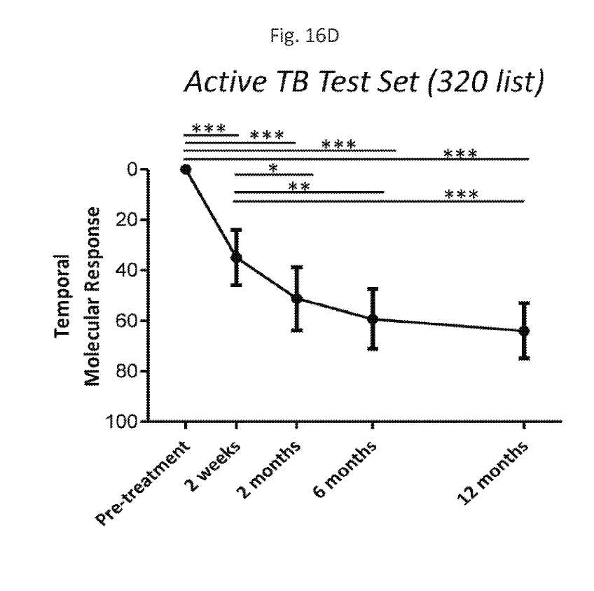

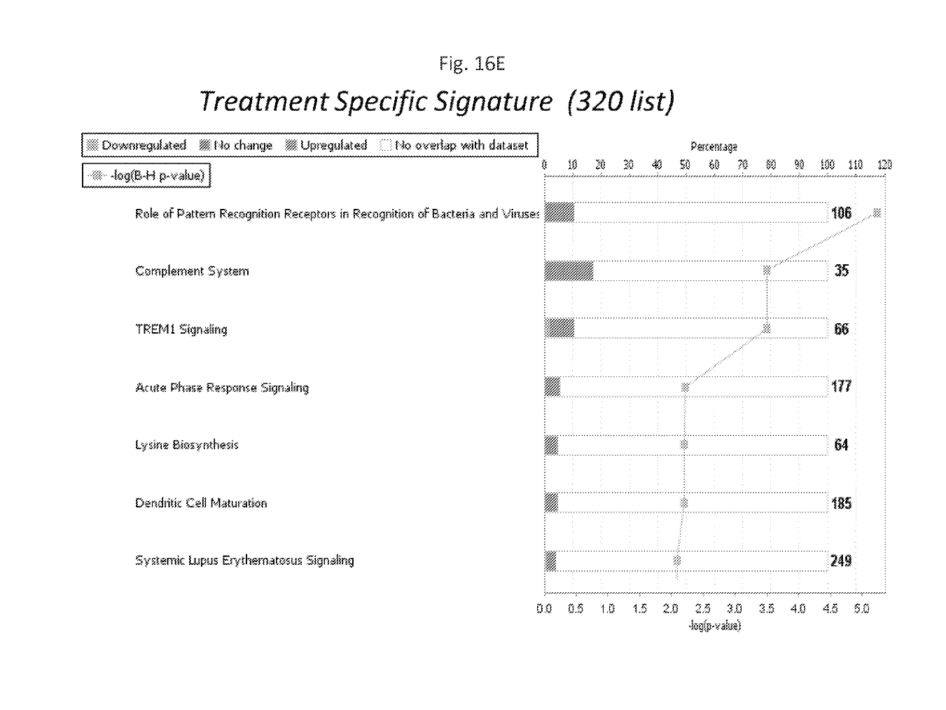

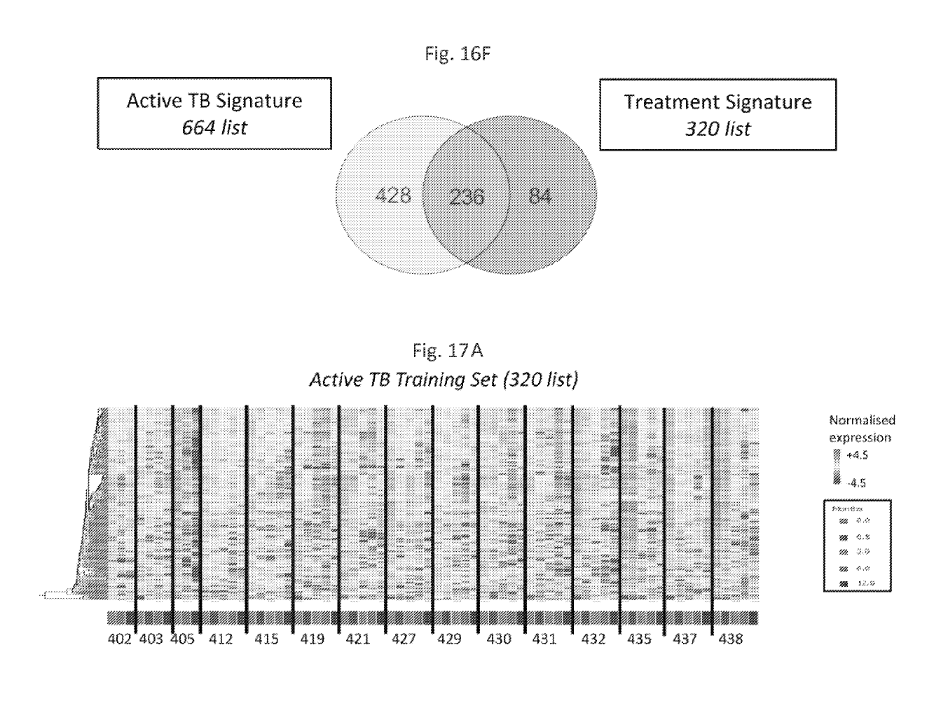

A Specific TB Treatment Response Signature Also Significantly Diminishes at Two Weeks Post Treatment. Defining transcriptional signature that specifically reflected the patients' response to clinically successful anti-TB treatment (comparing time points zero and six months) was next sought. To determine this treatment specific signature, a computer algorithm was first used to randomise the South Africa 2011 cohort into two groups of patients [12] (FIG. 11A). This allowed the signature from one group of patients (active TB Training Set) to be derived and then the findings in another independent group of patients (active TB Test Set) to be validated. 320 transcripts (Table 12) were found to be significantly differentially expressed between the pre-treatment active TB Training Set samples and their paired six-month treated samples (FIG. 16A). The treatment specific 320-transcript signature was shown to rapidly and significantly change at two weeks onwards after treatment initiation, in the active TB Training set (FIGS. 16A and 16B). This was validated in the active TB Test Set (FIGS. 16C and 16D). In both cohorts the change in the temporal molecular response was significant at two weeks post-treatment (FIGS. 16B and 16D). Analysis of the 320 transcripts by IPA indicated the most significantly represented pathways were related to the innate immune pathways, encompassing genes related to complement and Toll-like receptors (FIG. 16E). 74% of genes present in the treatment specific 320-transcript signature were also contained the active TB 664-transcript signature (FIG. 16F).

Although by applying the temporal molecular response it was observed that the treatment specific 320-transcript signature changed significantly between two weeks and six months post treatment initiation, this was no longer apparent between two months and six months post treatment initiation (FIGS. 3B and D). This could suggest that the transcriptional response reaches a plateau at two months and therefore the two month gene expression profiles would not be significantly different from the latent TB expression profiles. To establish whether any significant changes occurred between two months and the latent TB patients, each of the time points was compared: two, six and 12 months to the latent TB profiles. It was determined that 96 transcripts were significantly differentially expressed between two months and latent TB (Mann Whitney paired Benjamini Hochberg p<0.01, data not shown). Ingenuity Pathway Analysis demonstrated the top three significant pathways associated with the 96 transcripts were ‘role of NFAT in regulation of the immune response’, ‘integrin signaling’ and ‘primary immunodeficiency signaling’ (data not shown). However no genes were significantly differentially expressed between six & 12 months, six months & latent TB, and 12 months & latent TB (Mann Whitney paired Benjamini Hochberg p>0.01).

Measuring an Individual Patient's Transcriptional Response to Anti-TB Treatment. Each patient's discrete treatment specific response (320 transcripts) is shown in the heatmaps of FIGS. 14A & 17A and using the temporal molecular responses in FIGS. 14B & 17B. All 29 patients in the active TB treated cohort had a rapid and early positive temporal response after two weeks of treatment. Interestingly, not all the individual transcriptional responses were identical (FIGS. 14A & 17A) as demonstrated by the quantitative scoring provided by the temporal molecular responses (FIGS. 14B & 17B).

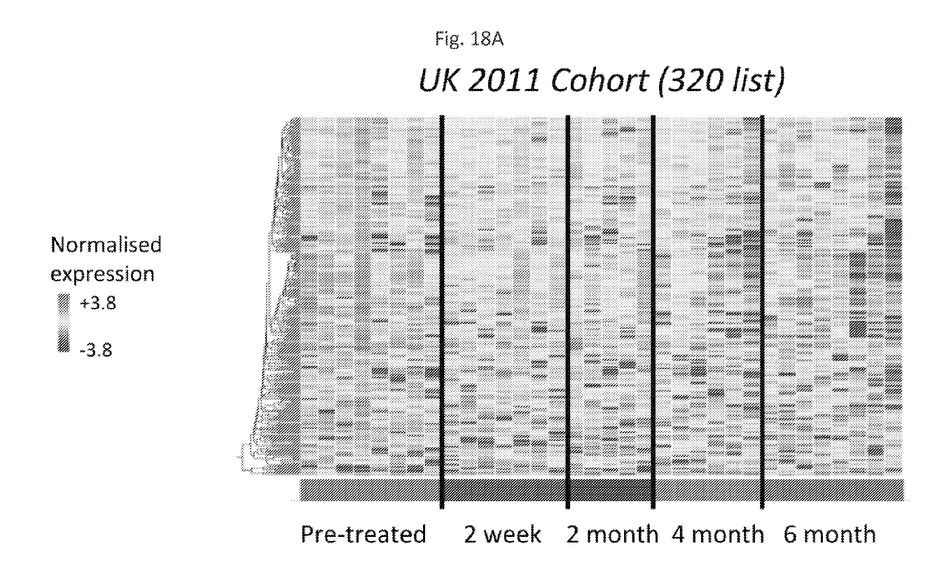

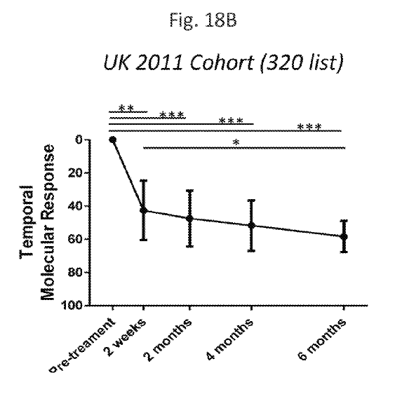

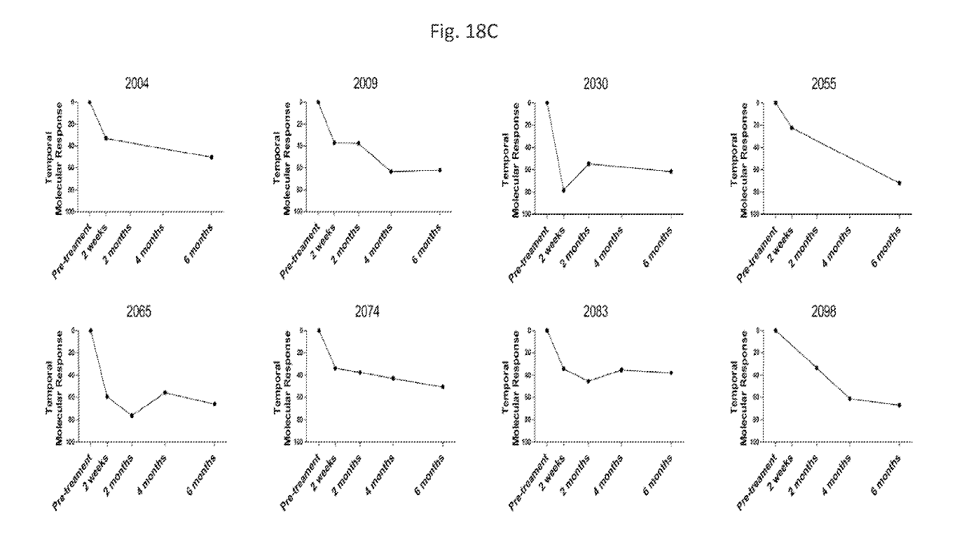

To determine whether the significant change in the treatment specific 320-transcript signature that had been demonstrated in a South African cohort was also applicable to patients in an intermediate burden setting, the 320-transcript signature was tested in a UK cohort. As observed in the South African cohort, the signature was rapidly and significantly diminished from two weeks post-treatment initiation (FIGS. 18A and 18B). The changes in the blood transcriptional response could be clearly quantified in individual patients as shown by the temporal molecular response (FIG. 18C). The significant transcriptional blood change correlated with successful treatment of patients as assessed after six months by radiographic and clinical parameters (data not shown).

For additional validation that active-TB transcriptional signatures show significant changes as early as two weeks after treatment initiation, it was demonstrated that the active TB signatures (393- and 86-transcript signatures) from an earlier study [11], also significantly diminished after two weeks treatment, in the South Africa 2011 treated cohort (FIG. 12A-12C).

Discussion. As disclosed herein, a whole blood active-TB transcriptional signature was derived consisting of 664 transcripts capable of distinguishing untreated South African active TB patients from South African latent TB patients. It was demonstrated that this active-TB transcriptional signature significantly diminishes in active TB patients after just two weeks of initiation of clinically successful anti-TB treatment. In addition, it was demonstrated that a treatment-specific transcriptional signature, consisting of 320 transcripts, derived from comparing a cohort of South African untreated active TB samples to their paired six-month end-of-treatment samples, also significantly diminishes after just two weeks of anti-TB treatment. Furthermore the significant change in the treatment-specific signature was validated in two more clinically successfully treated cohorts, from the high TB-burden setting of South Africa and from the intermediate TB-burden setting of London, UK. Both the active-TB and treatment-specific transcriptional signatures were dominated by IFN signaling and innate immune response genes. The transcriptional response to anti-TB treatment could also be individually quantified for each patient. Together, these findings suggest that blood transcriptional signatures could be used as early surrogate biomarkers of a successful treatment response, in both the clinical setting and in drug development.

TB treatment monitoring is a major challenge for attempts to eradicate Mtb infection. In April 2010 the Centers for Disease Control and National Institutes of Health brought together experts in the field and research scientists with the sole purpose of addressing this problem [17]. Poor treatment monitoring, and hence inadequate treatment, leads to worsening of a patient's disease, increasing the potential for disease spread and the risk of developing drug resistant mycobacteria. Currently the two-month sputum culture conversion is the only biomarker of successful TB treatment [6]. However it is time consuming, taking several weeks to grow the bacilli and results can be compromised by contamination. Moreover patients who have clinically improved may be unable to expectorate sputum at two months and potentially incorrectly labeled as having a negative culture [18]. Furthermore, although sputum conversion is commonly used as a surrogate end point for treatment response in clinical trials evaluating new drugs, a systematic review and meta-analysis to assess its accuracy in predicting an individual's treatment failure revealed low sensitivity and only modest specificity [7], [19]. While other biomarkers have also been trialed, including C-reactive protein, IFN-γ and neopterin, all have similarly shown poor sensitivity and specificity [20]. Chest X-rays are commonly used in the clinical setting as a marker of treatment response but they generally improve slower than the clinical response and lack specificity as interpretation can be confounded by previous lung damage [18]. Moreover interpretation of radiographic changes in response to treatment has not yet been standardised, and the facilities are not always available in developing countries [8]. Therefore there is clearly a need for early and easily detectable biomarkers for treatment monitoring, capable of potentially identifying poor responses due to drug resistance or lack of treatment adherence, and available for patients unable to produce sputum.

In an earlier study, it was demonstrated in a small number of patients that blood transcriptional signatures in UK active TB patients diminished after two months of anti-TB treatment [11]. In study disclosed herein, a significant blood transcriptional response to anti-TB treatment has been shown to occur rapidly, as early as two weeks (FIGS. 12A, 12B, 12C, 13A, 13B, 13E, 13F, 14A, 14B, 16A, 16B, 16C, 16D, 17A, 17B, 18A, 18B, and 18C). This early transcriptional response could be as a consequence of the observed rapid and high killing capacity of antimycobacterial antibiotics leading to a substantial reduction in mycobacterial load [21], [22], [23]. Although the signatures derived may not be completely specific for active TB, since clinically similar diseases such as sarcoidosis show common transcripts [24], demonstration of a response to antimycobacterial therapy as shown herein, could help resolve this overlap and so improve diagnostic specificity.

With the disclosure of this Example, it is shown that the whole blood active-TB transcriptional signature is dominated by IFN signaling and innate immune response genes. These findings are in agreement with previous work [11], and with other gene expression studies in TB [25], [26]. This robust correlation occurring between different host populations, likely different Mtb strains, diverse environments and microarray analysis strategies indicates that blood transcriptomics may be developed into robust novel diagnostic tools. Furthermore, as demonstrated herein, the derived treatment specific 320-transcript signature also had many genes in common with the active TB 664-transcript signature (FIG. 3F). This overlap of genes may help in future development of gene subsets that correlate with a patient's response to anti-TB treatment, acting as a surrogate marker of treatment failure or success.

Due to the ethical design of this study, active TB patients who did not respond to TB treatment are not presented. But this study has demonstrated a very important proof-of-principle that active TB patients who are successfully treated have a dramatic measurable change in their blood gene expression profiles as early as two weeks. The use of a commercially available whole genome microarray platform together with broadly available bioinformatics analyses programmes should easily allow rapid validation in subsequent TB treatment studies, including a comparison with patients with MDR-TB and HIV/TB co-infected cohorts. This study focused on TB patients who are not co-infected with HIV, as they represent the majority of patients infected with Mtb. WHO 2010 reports that of the 1.4 million deaths, three-quarters were not known to be co-infected with HIV [2].

No other studies appear to have specifically derived transcriptional signatures of response to TB treatment. However, two other studies have described relevant treatment related transcriptional differences. Mistry et al. found that patients who had completed a course of anti-TB treatment displayed similar expression profiles to a latent TB group, but Mistry et al. did not examine any patients during their anti-TB treatment course, and Mistry et al. used custom arrays [27], making it therefore more difficult for others to validate. Joosten et al. showed in a small number of samples that their active TB gene set diminished after two months of anti-TB treatment; however they did not examine any patients at earlier timepoints [28]. The early TB treatment blood transcriptional signature disclosed herein has great potential for development in blood biomarkers for clinical use and could be measured in the field using a polymerase chain reaction assay, similar to the WHO endorsed GeneXpert MTB/RIF test already in use for TB diagnostics in both developing and developed countries. However a blood host biomarker, based on the transcriptional signature of the study disclosed herein, would have advantages over the GeneXpert test since it would not require sputum.

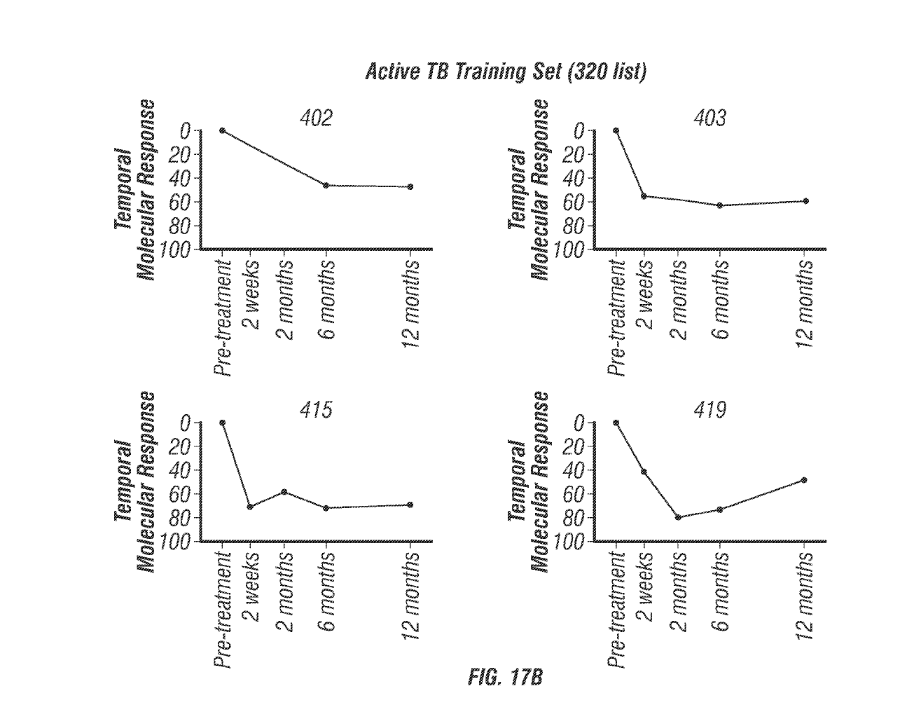

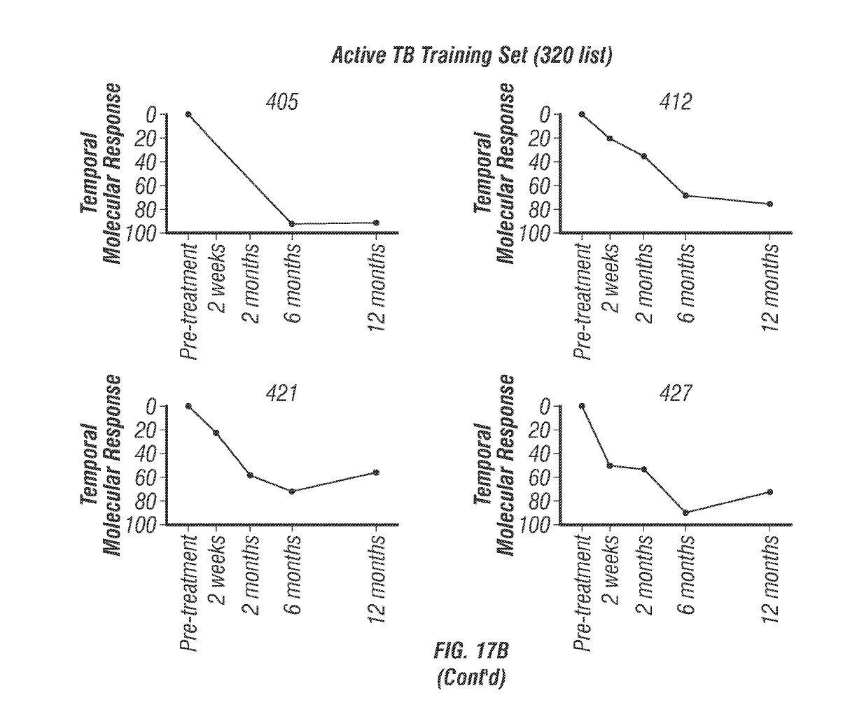

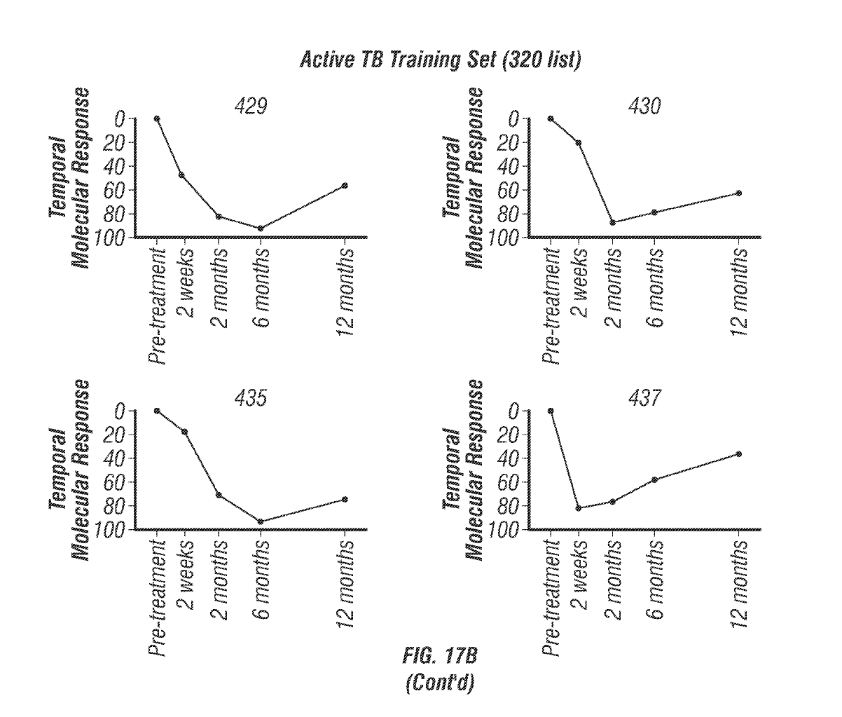

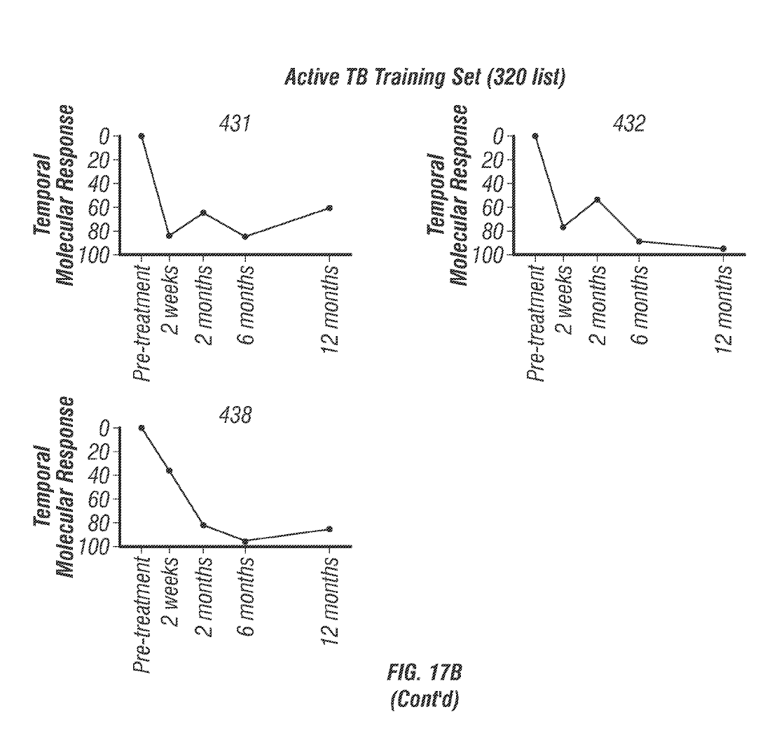

A further problem in the management of TB is the extended length of treatment, requiring a minimum duration of six months. However the treatment duration required for maximum efficacy and preventing resistance, has not been fully established. The ability therefore to stratify patients into groups requiring shorter or longer treatment durations, particularly in resource limited settings, could be of value in improving patient compliance and reducing treatment related side effects. We demonstrate here that some patient's transcriptional response appeared to plateau before six months (FIGS. 14A, 14B, 17A & 17B) suggesting blood transcriptional signatures may help develop personalized treatment regimes.

### Example 3

Use of a 320 gene transcriptional signature, as prominently disclosed in Example 2, was also prominently used for tests disclosed in this Example 3. In particular, data from preliminary studies demonstrate that blood-derived transcriptional signatures are diminished between three to six days after initiation of anti-TB treatment.

Materials and Methods. Recruitment methodology (i.e., inclusion and exclusion criteria) as detailed in the above Examples was not changed. For a small sample of six patients having symptoms of TB, blood was sampled every day for the first two weeks (depending on patient availability; i.e., missing data points for various days are due to patient unavailability). Three of these patients were later confirmed as having TB and as unambiguously meeting the previously-noted inclusion and exclusion criteria: Patient ID 2208; Patient ID 2220; and Patient ID 2232.

For RNA isolation and analysis, a few small methodology modifications were made to the methodologies of Example 2. First, sample RNA was isolated using 1 ml whole blood and the PerfectPure RNA Blood Kit (Invitrogen/Applied Biosystems/Ambion) according to the manufacturer's instructions. Second, 0.7-2.2 μg isolated total RNA was globin reduced using the GLOBINclear Human Kit (Invitrogen/Applied Biosystems/Ambion) according to the manufacturer's instructions. Third, raw data were processed using GeneSpring GX version 12 (Agilent Technologies).

In addition to generating heatmaps and temporal molecular response data from 320-transcript signatures for Patient ID 2208, Patient ID 2220, and Patient ID 2232, heatmaps and temporal molecular response data were generated for 393- and 86-transcript signatures for each of these patients using the methodologies of Example 2. In detail, 393- and 86-transcript signatures were translated from the HT-12 V3 BeadChip arrays to HT-12 V4 BeadChip arrays using GeneSpring GX version 11.5 (Agilent Technologies) and translated to slightly fewer probes in V4′ due to slight differences in probe sets between Illumina Human HT-12 V3 and V4 BeadChip versions. That is, data were also obtained for 380 list (i.e., Illumina Human HT-12 V4 BeadChip translation of Illumina Human HT-12 V3 BeadChip 393 list) and 83 list (i.e., Illumina Human HT-12 V4 BeadChip translation of Illumina Human HT-12 V3 BeadChip 86 list) treatment response profiles.

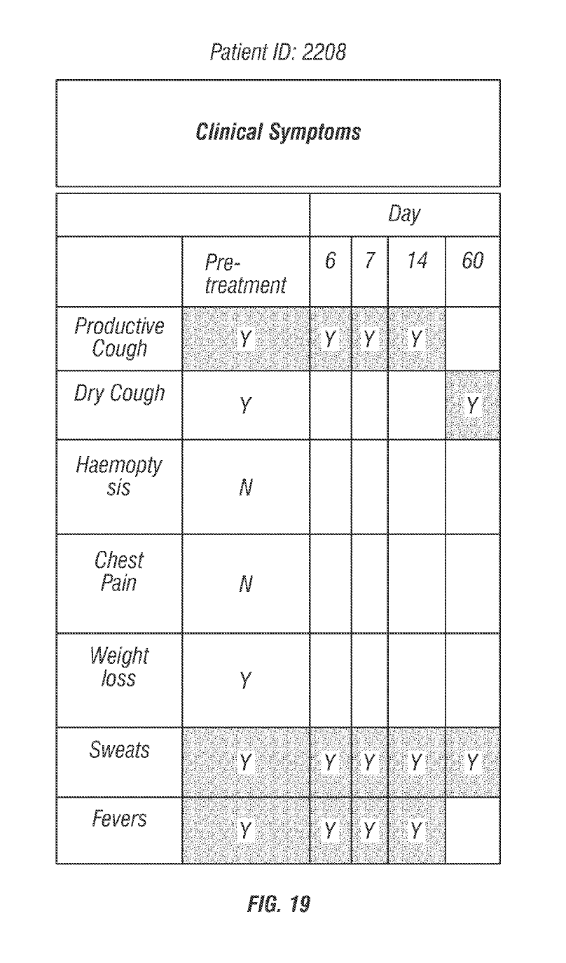

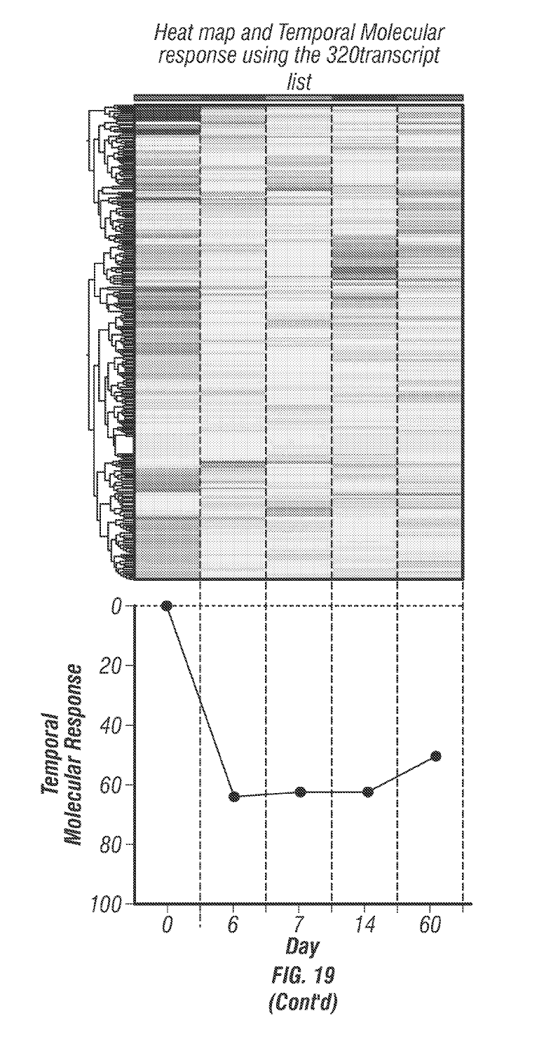

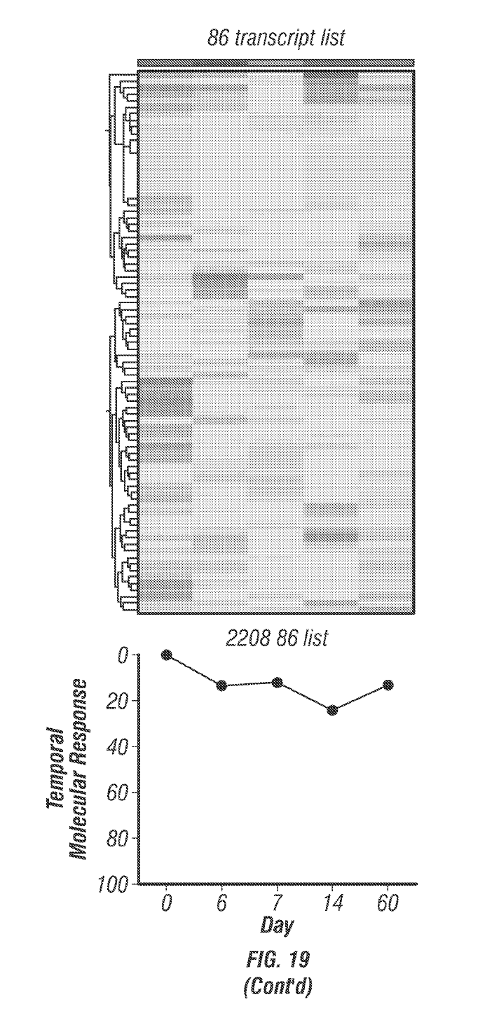

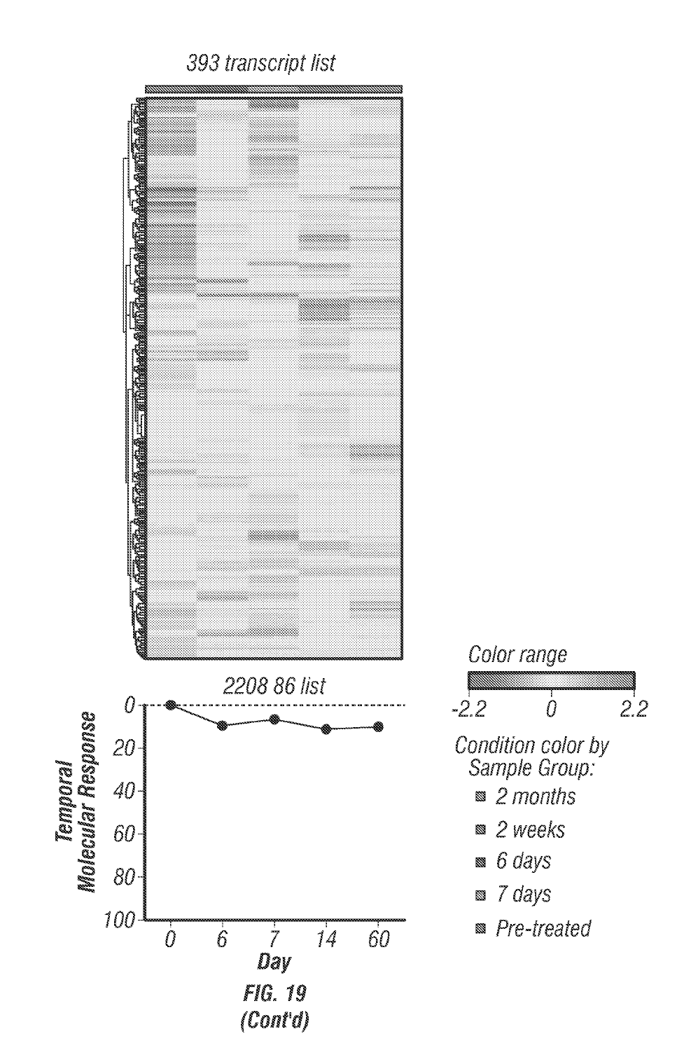

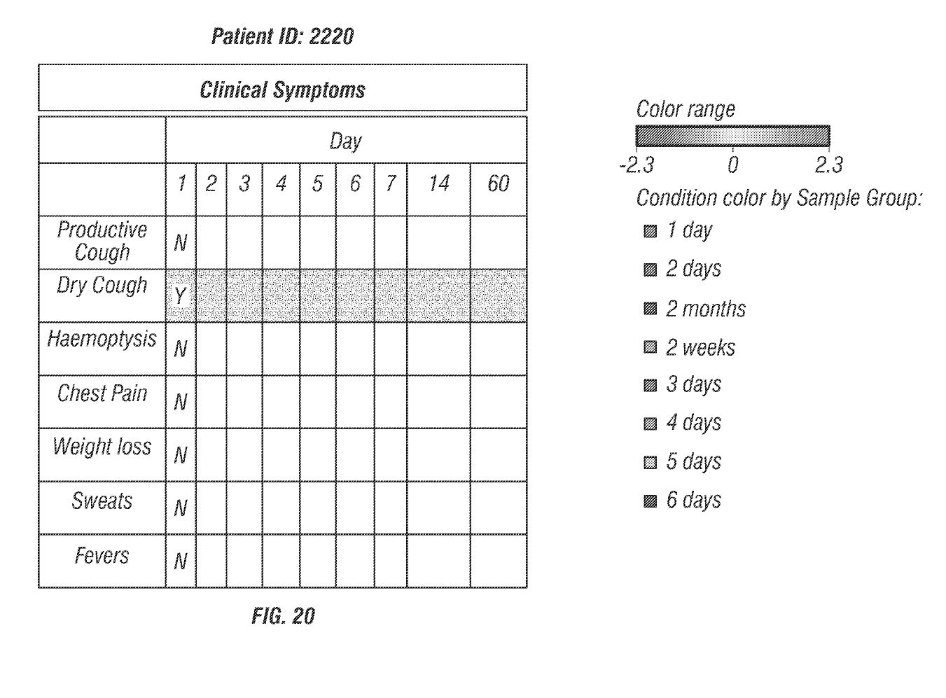

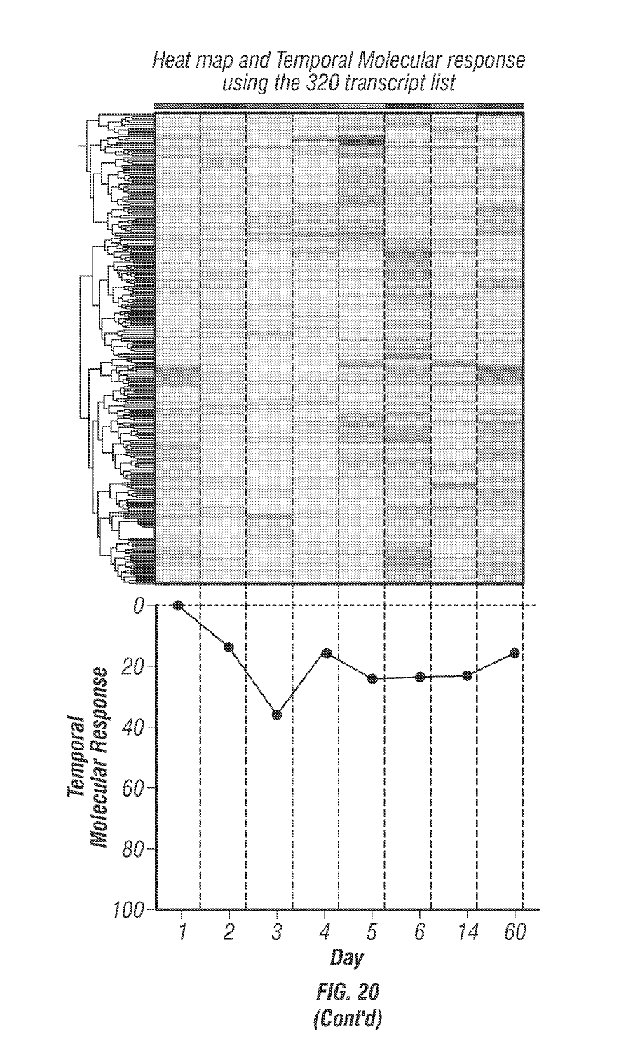

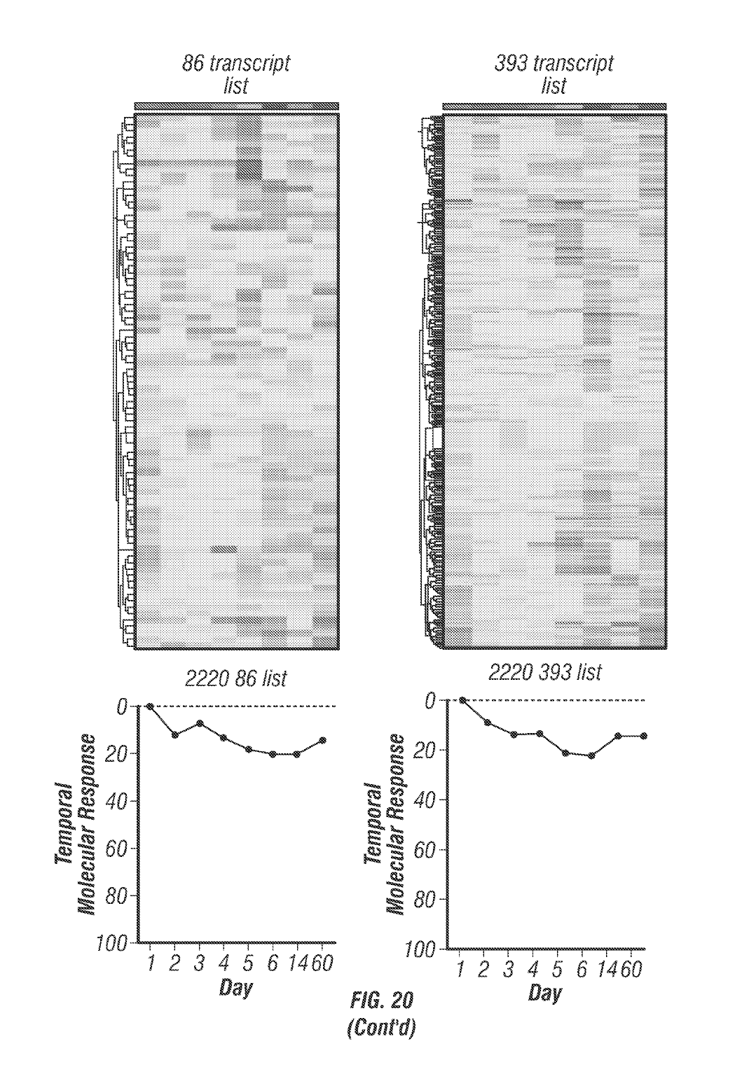

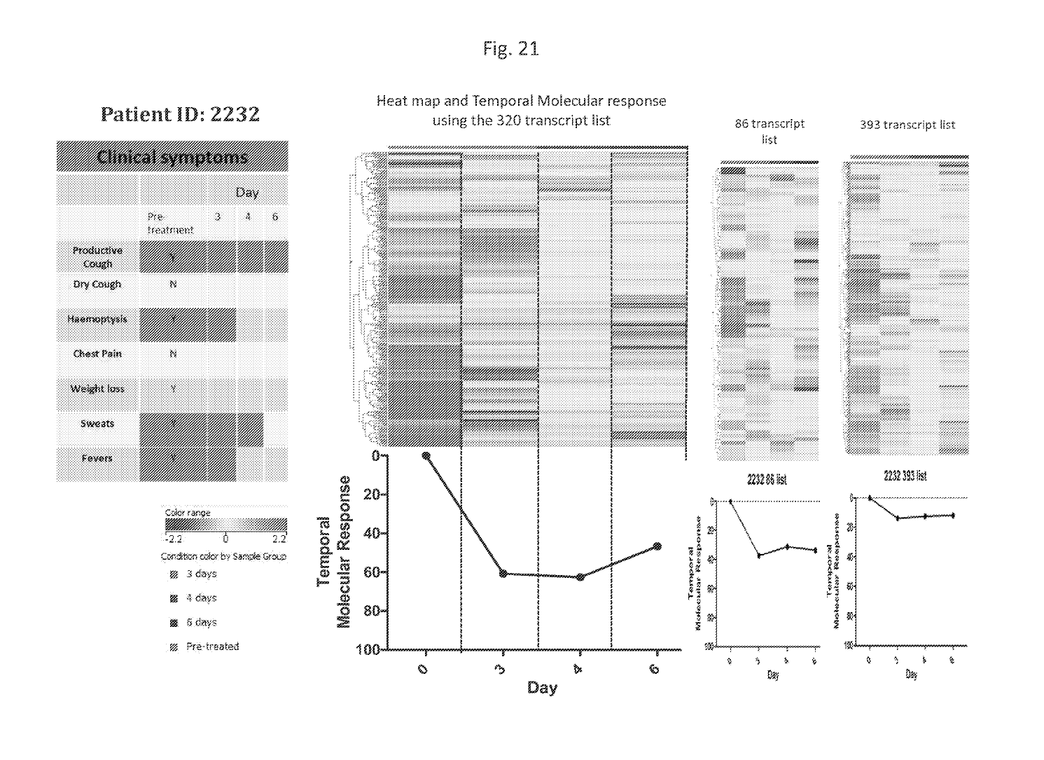

Results. Heatmaps and corresponding temporal molecular response data for Patient ID 2208, Patient ID 2220, and Patient 2232 are provided in FIGS. 19, 20, and 21, respectively, for 320, 86, and 393 transcript lists. For each of the three patients, less temporal molecular response is shown for either the 86 or the 393 transcript lists than for the 320 transcript list. This is consistent with the 393 and 86 transcript lists not being derived for treatment response. In contrast, temporal molecular response for the 320 transcript list is more pronounced. For example, the “Day 3” temporal molecular response data points for Patient ID 2220 and Patient ID 2232, i.e., the data point differences from the “0” baseline at “Day 3” for these patients, is more pronounced for the 320-transcript list than for either the 86- or 393-transcript lists.

In summary, transcriptional signatures, measured in easily accessible whole blood, showed a detectable response to anti-TB treatment, and this response was rapid and could be measured as early as two weeks (or, as preliminary data from Example 3 show, very much sooner) after initiation of treatment—far more quickly, and more consistently, than in currently available tests. In addition, this early response to anti-TB treatment was demonstrated in both high- and intermediate-burden settings. Transcriptional response could be measured for each individual TB patient, thus providing a potential clinical tool for single patient treatment monitoring. Furthermore, this monitoring promises to aid in patient stratification for treatment(s) with differing regimen lengths. These findings provide compelling evidence for a biomarker successful in assessing early anti-TB treatment response. This biomarker of early treatment response would allow rapid detection of both inadequate treatment regimens and poor treatment compliance, and therefore shows particular usefulness for reducing the spread of TB as brought about through the generation and spread of drug resistant Mtb.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference in the entirety of each to the same extent as if each individual publication or patent application was specifically and individually indicated to be incorporated by reference in its entirety.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the teem “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. As used herein, the phrase “consisting essentially of” limits the scope of a claim to the specified materials or steps and those that do not materially affect the basic and novel characteristic(s) of the claimed invention. As used herein, the phrase “consisting of” excludes any element, step, or ingredient not specified in the claim except for, e.g., impurities ordinarily associated with the element or limitation.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

TABLES

TABLE 1
Genes Present in the Top Significantly Represented
Ingenuity Pathways (Untreated South Africa 2011 Cohort)(Ingenuity
Pathway Analysis of 664 Transcripts)

| | | Altered T&B Cell | | Role of PRRs in | |
|---|---|---|---|---|---|
| IFN | | Signaling in | | Recognition of | |
| Signaling | | Rheumatoid Arthritis | | Viruses and Bacteria | |
| | Fold | | Fold | | Fold |
| Gene | Change | Gene | Change | Gene | Change |
| IFI35 | 2.32 | CD40LG | −3.09 | C5 | 2.45 |
| IFIT3 | 5.81 | CD79A | −5.09 | C1QB | 24.63 |
| IFITM1 | 2.39 | CD79B | −2.15 | C1QC | 4.66 |
| IRF1 | 2.26 | FAS | 2.43 | CASP1 | 2.11 |
| JAK2 | 2.38 | FCER1G | 2.05 | IFIH1 | 2.39 |
| SOCS1 | 3.33 | IL15 | 5.53 | IL1B | 2.29 |
| STAT1 | 3.40 | IL1B | 2.29 | IRF7 | 2.25 |
| TAP1 | 2.42 | IL1RN | 2.06 | NLRC4 | 3.17 |
| | | SLAMF1 | −2.84 | NOD2 | 2.50 |
| | | TLR2 | 3.12 | TLR2 | 3.12 |
| | | TLR5 | 3.07 | TLR5 | 3.07 |
| | | TNFSF13B | 2.36 | | |

| | | Role of Macrophages, | | | |
|---|---|---|---|---|---|
| TREM1 | | Fibroblasts, Endothelial Cells | | SLE | |
| Signaling | | in Rheumatoid Arthritis | | Signaling | |
| | Fold | | Fold | | Fold |
| Gene | Change | Gene | Change | Gene | Change |
| CASP1 | 2.11 | C5 | 2.45 | C5 | 2.45 |
| CASP5 | 8.58 | CREB5 | 3.70 | CD3E | −2.20 |
| IL1B | 2.29 | F2RL1 | 2.27 | CD40LG | −3.09 |
| ITGAX | 4.97 | FCGR1A | 10.37 | CD79A | −5.09 |
| JAK2 | 2.38 | IL15 | 5.53 | CD79B | −2.15 |
| NOD2 | 2.50 | IL18R1 | 2.41 | FCER1G | 2.05 |
| PLCG1 | −2.02 | IL18RAP | 2.32 | FCGR1A | 10.37 |
| TLR2 | 3.12 | IL1B | 2.29 | FCGR1B | 12.33 |
| TLR5 | 3.07 | IL1RN | 2.06 | FCGR1C | 9.66 |
| | | IRAK3 | 2.05 | FCGR2C | 5.03 |
| | | JAK2 | 2.38 | FCGR3B | 2.13 |
| | | MAPK14 | 5.04 | IL1B | 2.29 |
| | | NFAT5 | 2.56 | IL1RN | 2.06 |
| | | OSM | 2.76 | LCK | −2.22 |
| | | PDGFA | 2.16 | NFAT5 | 2.56 |
| | | PLCG1 | −2.02 | PLCG1 | −2.02 |
| | | SOCS1 | 3.33 | TNFSF13B | 2.36 |
| | | SOCS3 | 4.06 | | |
| | | TLR2 | 3.12 | | |
| | | TLR5 | 3.07 | | |
| | | TNFSF13B | 2.36 | | |
| | | TRAF5 | −2.12 | | |

TABLE 2A
South Africa Cohort

| | Untreated | Untreated | Treated |
|---|---|---|---|
| | Active TB | Latent TB | Active TB |
| | Cohort | Cohort | Cohort |
| Total | 29 | 38 | 20 |
| Gender (total males) | 19 | 17 | 12 |
| Ethnicity | 29 Black | 38 Black | 20 Black |
| | African | African | African |
| Age (ave. & range, yrs) | 34 (21-65) | 22 (18-44) | 36 (19-65) |
| Previous TB (total no.) | 6 | 0 | 3 |
| Productive cough (total no.) | 29 | 0 | 20 |
| Smear +ve (total no.) | 28 | N/A | 19 |
| Night sweats (total no.) | 25 | 0 | 22 |
| Weight loss (total no.) | 28 | 0 | 19 |
| Chest x-ray performed | 1 (1/1 abnormal) | Not done | 0 |
| (total no.) | | | |

TABLE 2B
UK 2011 Cohort

| | Treated Active TB Cohort |
|---|---|
| Total | 8 |
| Gender (total males) | 3 |
| Ethnicity | 2 Black African, 3 Indian subcont., |
| | 2 SE Asian, 1 Caucasian |
| Age (ave. & range, yrs) | 33 (19-67) |
| Previous TB (total no.) | 0 |
| Productive cough (total no.) | 6 |
| Smear +ve (total no.) | 4 |
| Night sweats (total no.) | 4 |
| Weight loss (total no.) | 4 |
| Chest x-ray performed (total no.) | 8 (7/8 abnormal) |

TABLE 3
Genes Present in the Top Significantly Represented
Ingenuity Pathway (UK & South Africa 2009 Cohorts).
SOUTH AFRICA 2009 (711)

| | |
|---|---|
| IFN Signaling | Complement System |

| Symbol | Fold Change | Symbol | Fold Change |
|---|---|---|---|
| IFI35 | 2.514 | C2 | 2.127 |
| IFIT1 | 2.143 | C1QB | 6.215 |
| IFIT3 | 4.149 | C1QC | 7.875 |
| IFITM1 | 2.155 | C4BPA | 2.675 |
| OAS1 | 2.476 | CD59 | 2.535 |
| SOCS1 | 4.038 | CR1 | 2.443 |
| STAT1 | 3.063 | SERPING1 | 10.744 |
| TAP1 | 2.205 | | |

| | |
|---|---|
| Altered T & B | Role of Pattern Recognition |
| Cell Signaling in | Receptors in Recognition of |
| Rheumatoid Arthritis | Bacteria and Viruses |

| Symbol | Fold Change | Symbol | Fold Change |
|---|---|---|---|
| CD79A | −4.048 | C1QB | 6.215 |
| CD79B | −2.100 | C1QC | 7.875 |
| FAS | 2.096 | CASP1 | 2.024 |
| FCER1G | 2.026 | IL1B | 2.052 |
| IL15 | 2.609 | IRF7 | 2.021 |
| IL1B | 2.052 | NLRC4 | 2.039 |
| IL23A | −2.253 | OAS1 | 2.476 |
| TLR5 | 2.885 | OAS3 | 2.578 |
| TNFRSF13B | −2.693 | TLR5 | 2.885 |
| TNFSF13B | 2.115 | | |

| | |
|---|---|
| Role of JAK Family | Communication Between |
| Kinases in IL-6 Type | Innate & Adaptive |
| Cytokine Signaling | Immune Cells |

| | Symbol | Fold Change | Symbol | Fold Change |
|---|---|---|---|---|
| | MAPK14 | 2.591 | | |
| | OSM | 2.514 | | |
| | SOCS1 | 4.038 | | |
| | SOCS3 | 3.147 | | |
| | STAT1 | 3.063 | | |
| Dendritic Cell Maturation | | | | |
| | Symbol | Fold Change | | |

UK TRAINING 2009 (565)

| | |
|---|---|
| IFN Signaling | Complement System |

| Symbol | Fold Change | Symbol | Fold Change |
|---|---|---|---|
| IFI35 | 2.685 | | |
| IFIT1 | 3.071 | | |
| IFIT3 | 5.317 | | |
| IFITM1 | 2.014 | | |
| JAK2 | 2.424 | | |
| OAS1 | 3.454 | | |
| SOCS1 | 2.960 | | |
| STAT1 | 2.900 | | |
| STAT2 | 2.079 | | |
| TAP1 | 2.291 | | |

| | |
|---|---|
| Altered T & B | Role of Pattern Recognition |
| Cell Signaling in | Receptors in Recognition of |
| Rheumatoid Arthritis | Bacteria and Viruses |

| Symbol | Fold Change | Symbol | Fold Change |
|---|---|---|---|
| CD86 | −2.077 | C1QB | 9.514 |
| CD40LG | −2.021 | C3AR1 | 2.065 |
| CD79A | −2.051 | CASP1 | 2.146 |
| FAS | 2.185 | IFIH1 | 2.027 |
| FCER1G | 2.002 | IL1B | 2.037 |
| IL15 | 2.599 | IRF7 | 2.438 |
| IL1B | 2.037 | NLRC4 | 2.276 |
| IL1RN | 2.297 | OAS1 | 3.454 |
| IL23A | −2.964 | OAS2 | 3.335 |
| TLR2 | 2.005 | OAS3 | 3.246 |
| TLR5 | 2.346 | TLR2 | 2.005 |
| TLR8 | 2.189 | TLR5 | 2.346 |
| TNFRSF13B | −2.714 | TLR8 | 2.189 |
| TNFSF13B | 2.025 | | |

| | |
|---|---|
| Role of JAK Family | Communication Between |
| Kinases in IL-6 Type | Innate & Adaptive |
| Cytokine Signaling | Immune Cells |

| | Symbol | Fold Change | Symbol | Fold Change |
|---|---|---|---|---|
| | | | CD86 | −2.077 |
| | | | CD40LG | −2.021 |
| | | | CXCL10 | 3.407 |
| | | | FCER1G | 2.002 |
| | | | IL15 | 2.599 |
| | | | IL1B | 2.037 |
| | | | IL1RN | 2.297 |
| | | | TLR2 | 2.005 |
| | | | TLR5 | 2.346 |
| | | | TLR8 | 2.189 |
| | | | TNFRSF13B | −2.714 |
| | | | TNFSF13B | 2.025 |
| Dendritic Cell | | | | |
| Maturation | | | | |
| | Symbol | Fold Change | | |
| | CD86 | −2.077 | | |
| | CD40LG | −2.021 | | |
| | CREB5 | 2.530 | | |
| | FCER1G | 2.002 | | |
| | FCGR1A | 6.840 | | |
| | FCGR1B | 7.042 | | |
| | IL15 | 2.599 | | |
| | IL1B | 2.037 | | |
| | IL1RN | 2.297 | | |
| | IL23A | −2.964 | | |
| | JAK2 | 2.424 | | |
| | MAPK14 | 3.174 | | |
| | STAT1 | 2.900 | | |
| | STAT2 | 2.079 | | |
| | TLR2 | 2.005 | | |

UK TEST 2009 (224)

| | |
|---|---|
| IFN Signaling | Complement System |

| Symbol | Fold Change | Symbol | Fold Change |
|---|---|---|---|
| IFIT3 | 2.899 | C2 | 2.419 |
| OAS1 | 2.257 | C1QB | 5.045 |
| SOCS1 | 2.296 | C1QC | 3.630 |
| STAT1 | 2.185 | SERPING1 | 5.462 |

TABLE 4
List of the 224 genes

| ProbeID | Symbol | Entrez_Gene_ID | Definition |
|---|---|---|---|
| 2710020 | | | BX093329 Soares_parathyroid_tumor_NbHPA Homo |
| | | | sapiens cDNA clone IMAGp998A124183; |
| | | | IMAGE: 1648403, mRNA sequence |
| 7550392 | GLUL | 2752 | Homo sapiens glutamate-ammonia ligase (glutamine |
| | | | synthetase) (GLUL), transcript variant 3, mRNA. |
| 2190349 | KCNJ15 | 3772 | Homo sapiens potassium inwardly-rectifying channel, |
| | | | subfamily J, member 15 (KCNJ15), transcript variant 3, |
| | | | mRNA. |
| 4120243 | PSG9 | 5678 | Homo sapiens pregnancy specific beta-1-glycoprotein 9 |
| | | | (PSG9), mRNA. |
| 5700725 | EPSTI1 | 94240 | Homo sapiens epithelial stromal interaction 1 (breast) |
| | | | (EPSTI1), transcript variant 2, mRNA. |
| 670594 | C2 | 717 | Homo sapiens complement component 2 (C2), mRNA. |
| 2070646 | GPR84 | 53831 | Homo sapiens G protein-coupled receptor 84 (GPR84), |
| | | | mRNA. |
| 130181 | ANKRD22 | 118932 | Homo sapiens ankyrin repeat domain 22 (ANKRD22), |
| | | | mRNA. |
| 5860075 | CAMP | 820 | Homo sapiens cathelicidin antimicrobial peptide |
| | | | (CAMP), mRNA. |
| 6480246 | LOC649095 | 649095 | PREDICTED: Homo sapiens hypothetical LOC649095 |
| | | | (LOC649095), mRNA. |
| 4290368 | PSTPIP2 | 9050 | Homo sapiens proline-serine-threonine phosphatase |
| | | | interacting protein 2 (PSTPIP2), mRNA. |
| 2360348 | CMPK2 | 129607 | Homo sapiens cytidine monophosphate (UMP-CMP) |
| | | | kinase 2, mitochondrial (CMPK2), nuclear gene |
| | | | encoding mitochondrial protein, mRNA. |
| 2630195 | VAMP5 | 10791 | Homo sapiens vesicle-associated membrane protein 5 |
| | | | (myobrevin) (VAMP5), mRNA. |
| 2370228 | CCDC65 | 85478 | Homo sapiens coiled-coil domain containing 65 |
| | | | (CCDC65), mRNA. |
| 6620209 | FCGR1B | 2210 | Homo sapiens Fc fragment of IgG, high affinity Ib, |
| | | | receptor (CD64) (FCGR1B), transcript variant 2, |
| | | | mRNA. |
| 130609 | FCGBP | 8857 | Homo sapiens Fc fragment of IgG binding protein |
| | | | (FCGBP), mRNA. |
| 1510364 | GBP5 | 115362 | Homo sapiens guanylate binding protein 5 (GBP5), |
| | | | mRNA. |
| 6370768 | ETV7 | 51513 | Homo sapiens ets variant 7 (ETV7), mRNA. |
| 3440095 | PSG3 | 5671 | Homo sapiens pregnancy specific beta-1-glycoprotein 3 |
| | | | (PSG3), mRNA. |
| 4050039 | FCGR3A | 2214 | Homo sapiens Fc fragment of IgG, low affinity IIIa, |
| | | | receptor (CD16a) (FCGR3A), mRNA. |
| 70730 | GAS6 | 2621 | Homo sapiens growth arrest-specific 6 (GAS6), mRNA. |
| 1470091 | IL15 | 3600 | Homo sapiens interleukin 15 (IL15), transcript variant |
| | | | 1, mRNA. |
| 2490315 | | | Homo sapiens cDNA: FLJ23098 fis, clone LNG07440 |
| 6040577 | SLAMF8 | 56833 | Homo sapiens SLAM family member 8 (SLAMF8), |
| | | | mRNA. |
| 2760500 | CD38 | 952 | Homo sapiens CD38 molecule (CD38), mRNA. |
| 2680161 | GGTL3 | 2686 | Homo sapiens gamma-glutamyltransferase-like 3 |
| | | | (GGTL3), transcript variant 2, mRNA. |
| 2030309 | SERPING1 | 710 | Homo sapiens serpin peptidase inhibitor, clade G (C1 |
| | | | inhibitor), member 1 (SERPING1), transcript variant 2, |
| | | | mRNA. |
| 3170273 | FER1L3 | 26509 | Homo sapiens fer-1-like 3, myoferlin (C. elegans) |
| | | | (FER1L3), transcript variant 2, mRNA. |
| 580343 | ZNF540 | 163255 | Homo sapiens zinc finger protein 540 (ZNF540), |
| | | | mRNA. |
| 4920612 | GNLY | 10578 | Homo sapiens granulysin (GNLY), transcript variant |
| | | | 519, mRNA. |
| 4880333 | FLJ40504 | 284085 | Homo sapiens hypothetical protein FLJ40504 |
| | | | (FLJ40504), mRNA. |
| 50132 | ALS2CR16 | 130029 | PREDICTED: Homo sapiens amyotrophic lateral |
| | | | sclerosis 2 (juvenile) chromosome region, candidate 16 |
| | | | (ALS2CR16), mRNA. |
| 1110039 | CEP68 | 23177 | Homo sapiens centrosomal protein 68 kDa (CEP68), |
| | | | mRNA. |
| 5270403 | | | Homo sapiens cDNA FLJ20012 fis, clone ADKA03438 |
| 7160164 | MATK | 4145 | Homo sapiens megakaryocyte-associated tyrosine |
| | | | kinase (MATK), transcript variant 1, mRNA. |
| 6840035 | GBP1 | 2633 | Homo sapiens guanylate binding protein 1, interferon- |
| | | | inducible, 67 kDa (GBP1), mRNA. |
| 2190148 | GBP1 | 2633 | Homo sapiens guanylate binding protein 1, interferon- |
| | | | inducible, 67 kDa (GBP1), mRNA. |
| 150730 | ANKRD36 | 375248 | Homo sapiens ankyrin repeat domain 36 (ANKRD36), |
| | | | mRNA. |
| 240053 | GCH1 | 2643 | Homo sapiens GTP cyclohydrolase 1 (GCH1), |
| | | | transcript variant 4, mRNA. |
| 3800398 | FBXO6 | 26270 | Homo sapiens F-box protein 6 (FBXO6), mRNA. |
| 650753 | AFF3 | 3899 | Homo sapiens AF4/FMR2 family, member 3 (AFF3), |
| | | | transcript variant 2, mRNA. |
| 6220332 | SLC6A12 | 6539 | Homo sapiens solute carrier family 6 (neurotransmitter |
| | | | transporter, betaine/GABA), member 12 (SLC6A12), |
| | | | mRNA. |
| 4890270 | LY6E | 4061 | Homo sapiens lymphocyte antigen 6 complex, locus E |
| | | | (LY6E), mRNA. |
| 2760019 | | | Homo sapiens cDNA FLJ46319 fis, clone |
| | | | TESTI4042420 |
| 940356 | IL15RA | 3601 | Homo sapiens interleukin 15 receptor, alpha (IL15RA), |
| | | | transcript variant 1, mRNA. |
| 3870458 | BRDG1 | 26228 | Homo sapiens BCR downstream signaling 1 (BRDG1), |
| | | | mRNA. |
| 4730059 | BATF2 | 116071 | Homo sapiens basic leucine zipper transcription factor, |
| | | | ATF-like 2 (BATF2), mRNA. |
| 840709 | EIF2AK3 | 9451 | Homo sapiens eukaryotic translation initiation factor 2- |
| | | | alpha kinase 3 (EIF2AK3), mRNA. |
| 2070114 | CLEC4D | 338339 | Homo sapiens C-type lectin domain family 4, member |
| | | | D (CLEC4D), mRNA. |
| 7510647 | CACNA1E | 777 | Homo sapiens calcium channel, voltage-dependent, R |
| | | | type, alpha 1E subunit (CACNA1E), mRNA. |
| 2000661 | NCAPH2 | 29781 | Homo sapiens non-SMC condensin II complex, subunit |
| | | | H2 (NCAPH2), transcript variant 2, mRNA. |
| 2970286 | CEACAM1 | 634 | Homo sapiens carcinoembryonic antigen-related cell |
| | | | adhesion molecule 1 (biliary glycoprotein) |
| | | | (CEACAM1), transcript variant 2, mRNA. |
| 5700753 | CEACAM1 | 634 | Homo sapiens carcinoembryonic antigen-related cell |
| | | | adhesion molecule 1 (biliary glycoprotein) |
| | | | (CEACAM1), transcript variant 2, mRNA. |
| 1240270 | LMTK2 | 22853 | Homo sapiens lemur tyrosine kinase 2 (LMTK2), |
| | | | mRNA. |
| 1580435 | TGM2 | 7052 | Homo sapiens transglutaminase 2 (C polypeptide, |
| | | | protein-glutamine-gamma-glutamyltransferase) |
| | | | (TGM2), transcript variant 1, mRNA. |
| 3420593 | LMNB1 | 4001 | Homo sapiens lamin B1 (LMNB1), mRNA. |
| 4780128 | ATF3 | 467 | Homo sapiens activating transcription factor 3 (ATF3), |
| | | | transcript variant 4, mRNA. |
| 2640025 | HP | 3240 | Homo sapiens haptoglobin (HP), mRNA. |
| 6330471 | BLK | 640 | Homo sapiens B lymphoid tyrosine kinase (BLK), |
| | | | mRNA. |
| 290661 | CLN8 | 2055 | Homo sapiens ceroid-lipofuscinosis, neuronal 8 |
| | | | (epilepsy, progressive with mental retardation) (CLN8), |
| | | | mRNA. |
| 3180731 | MGST1 | 4257 | Homo sapiens microsomal glutathione S-transferase 1 |
| | | | (MGST1), transcript variant 1b, mRNA. |
| 4810079 | KIAA1632 | 57724 | Homo sapiens KIAA1632 (KIAA1632), mRNA. |
| 6040653 | TRIM6 | 117854 | Homo sapiens tripartite motif-containing 6 (TRIM6), |
| | | | transcript variant 2, mRNA. |
| 3610681 | OR2A9P | 441295 | Homo sapiens olfactory receptor, family 2, subfamily |
| | | | A, member 9 pseudogene (OR2A9P), non-coding RNA. |
| 5810086 | PHOSPHO2 | 493911 | Homo sapiens phosphatase, orphan 2 (PHOSPHO2), |
| | | | mRNA. |
| 130274 | LCK | 3932 | Homo sapiens lymphocyte-specific protein tyrosine |
| | | | kinase (LCK), transcript variant 2, mRNA. |
| 5890564 | ANKDD1A | 348094 | Homo sapiens ankyrin repeat and death domain |
| | | | containing 1A (ANKDD1A), mRNA. |
| 6760681 | LOC650761 | 650761 | PREDICTED: Homo sapiens similar to T-cell receptor |
| | | | alpha chain V region CTL-L17 precursor |
| | | | (LOC650761), mRNA. |
| 2690477 | LOC648710 | 648710 | PREDICTED: Homo sapiens similar to leukocyte |
| | | | immunoglobulin-like receptor, subfamily B (with TM |
| | | | and ITIM domains), member 3 (LOC648710), mRNA. |
| 1050215 | KCNJ15 | 3772 | Homo sapiens potassium inwardly-rectifying channel, |
| | | | subfamily J, member 15 (KCNJ15), transcript variant 1, |
| | | | mRNA. |
| 1240037 | DKFZP564O0823 | 25849 | Homo sapiens DKFZP564O0823 protein |
| | | | (DKFZP564O0823), mRNA. |
| 620403 | LOC400759 | 400759 | Homo sapiens similar to Interferon-induced guanylate- |
| | | | binding protein 1 (GTP-binding protein 1) (Guanine |
| | | | nucleotide-binding protein 1) (HuGBP-1) (LOC400759) |
| | | | on chromosome 1. |
| 780079 | LOC654053 | 654053 | PREDICTED: Homo sapiens similar to hypothetical |
| | | | LOC389634 (LOC654053), mRNA. |
| 1110592 | EBF1 | 1879 | Homo sapiens early B-cell factor 1 (EBF1), mRNA. |
| 3180392 | MARCO | 8685 | Homo sapiens macrophage receptor with collagenous |
| | | | structure (MARCO), mRNA. |
| 3520102 | CCL4L1 | 9560 | Homo sapiens chemokine (C-C motif) ligand 4-like 1 |
| | | | (CCL4L1), mRNA. |
| 130753 | AIM2 | 9447 | Homo sapiens absent in melanoma 2 (AIM2), mRNA. |
| 1260270 | AIM2 | 9447 | Homo sapiens absent in melanoma 2 (AIM2), mRNA. |
| 6400364 | NR4A2 | 4929 | Homo sapiens nuclear receptor subfamily 4, group A, |
| | | | member 2 (NR4A2), transcript variant 1, mRNA. |
| 5390427 | NAIP | 4671 | Homo sapiens NLR family, apoptosis inhibitory protein |
| | | | (NAIP), transcript variant 1, mRNA. |
| 5340240 | NAIP | 4671 | Homo sapiens NLR family, apoptosis inhibitory protein |
| | | | (NAIP), transcript variant 1, mRNA. |
| 7100338 | MMP28 | 79148 | Homo sapiens matrix metallopeptidase 28 (MMP28), |
| | | | transcript variant 3, mRNA. |
| 2750475 | D4S234E | 27065 | Homo sapiens DNA segment on chromosome 4 |
| | | | (unique) 234 expressed sequence (D4S234E), transcript |
| | | | variant 2, mRNA. |
| 4610136 | GPATCH4 | 54865 | Homo sapiens G patch domain containing 4 |
| | | | (GPATCH4), transcript variant 2, mRNA. |
| 1010360 | GCH1 | 2643 | Homo sapiens GTP cyclohydrolase 1 (GCH1), |
| | | | transcript variant 3, mRNA. |
| 4010270 | LOC440731 | 440731 | PREDICTED: Homo sapiens hypothetical LOC440731, |
| | | | transcript variant 2 (LOC440731), mRNA. |
| 3850435 | AGPAT3 | 56894 | Homo sapiens 1-acylglycerol-3-phosphate O- |
| | | | acyltransferase 3 (AGPAT3), mRNA. |
| 5220524 | HPCAL4 | 51440 | Homo sapiens hippocalcin like 4 (HPCAL4), mRNA. |
| 5910019 | C1QB | 713 | Homo sapiens complement component 1, q |
| | | | subcomponent, B chain (C1QB), mRNA. |
| 4640309 | | | EST391381 MAGE resequences, MAGP Homo sapiens |
| | | | cDNA, mRNA sequence |
| 2710709 | FCGR1B | 2210 | Homo sapiens Fc fragment of IgG, high affinity Ib, |
| | | | receptor (CD64) (FCGR1B), transcript variant 1, |
| | | | mRNA. |
| 1260482 | GZMK | 3003 | Homo sapiens granzyme K (granzyme 3; tryptase II) |
| | | | (GZMK), mRNA. |
| 3930368 | INCA | 440068 | Homo sapiens inhibitory caspase recruitment domain |
| | | | (CARD) protein (INCA), mRNA. |
| 520463 | GPER | 2852 | Homo sapiens G protein-coupled estrogen receptor 1 |
| | | | (GPER), transcript variant 3, mRNA. |
| 360132 | LHFPL2 | 10184 | Homo sapiens lipoma HMGIC fusion partner-like 2 |
| | | | (LHFPL2), mRNA. |
| 3990435 | | | BX282075 NIH_MGC_120 Homo sapiens cDNA clone |
| | | | IMAGp998M2011561; IMAGE: 5223355, mRNA |
| | | | sequence |
| 6350082 | FAM102A | 399665 | Homo sapiens family with sequence similarity 102, |
| | | | member A (FAM102A), transcript variant 1, mRNA. |
| 4610280 | MEIS3P1 | 4213 | Homo sapiens Meis homeobox 3 pseudogene 1 |
| | | | (MEIS3P1), non-coding RNA. |
| 6380136 | NFATC2IP | 84901 | PREDICTED: Homo sapiens nuclear factor of activated |
| | | | T-cells, cytoplasmic, calcineurin-dependent 2 |
| | | | interacting protein, transcript variant 3 (NFATC2IP), |
| | | | mRNA. |
| 3440630 | ESPN | 83715 | Homo sapiens espin (ESPN), mRNA. |
| 2710093 | ZNF74 | 7625 | Homo sapiens zinc finger protein 74 (ZNF74), |
| | | | transcript variant 1, mRNA. |
| 6420594 | TIFA | 92610 | Homo sapiens TRAF-interacting protein with forkhead- |
| | | | associated domain (TIFA), mRNA. |
| 1070367 | C19orf59 | 199675 | Homo sapiens chromosome 19 open reading frame 59 |
| | | | (C19orf59), mRNA. |
| 3890609 | PLSCR1 | 5359 | Homo sapiens phospholipid scramblase 1 (PLSCR1), |
| | | | mRNA. |
| 4150692 | PARP14 | 54625 | Homo sapiens poly (ADP-ribose) polymerase family, |
| | | | member 14 (PARP14), mRNA. |
| 780035 | SYTL2 | 54843 | Homo sapiens synaptotagmin-like 2 (SYTL2), |
| | | | transcript variant a, mRNA. |
| 990239 | KCNE3 | 10008 | Homo sapiens potassium voltage-gated channel, Isk- |
| | | | related family, member 3 (KCNE3), mRNA. |
| 10360 | OAS3 | 4940 | Homo sapiens 2′-5′-oligoadenylate synthetase 3, |
| | | | 100 kDa (OAS3), mRNA. |
| 990768 | OAS3 | 4940 | Homo sapiens 2′-5′-oligoadenylate synthetase 3, |
| | | | 100 kDa (OAS3), mRNA. |
| 3190521 | MS4A1 | 931 | Homo sapiens membrane-spanning 4-domains, |
| | | | subfamily A, member 1 (MS4A1), transcript variant 1, |
| | | | mRNA. |
| 5340767 | CEACAM1 | 634 | Homo sapiens carcinoembryonic antigen-related cell |
| | | | adhesion molecule 1 (biliary glycoprotein) |
| | | | (CEACAM1), transcript variant 1, mRNA. |
| 1940373 | ZNF831 | 128611 | Homo sapiens zinc finger protein 831 (ZNF831), |
| | | | mRNA. |
| 6380411 | IKZF3 | 22806 | Homo sapiens IKAROS family zinc finger 3 (Aiolos) |
| | | | (IKZF3), transcript variant 1, mRNA. |
| 580433 | DENND1A | 57706 | Homo sapiens DENN/MADD domain containing 1A |
| | | | (DENND1A), transcript variant 1, mRNA. |
| 3310376 | TIMM10 | 26519 | Homo sapiens translocase of inner mitochondrial |
| | | | membrane 10 homolog (yeast) (TIMM10), nuclear gene |
| | | | encoding mitochondrial protein, mRNA. |
| 5340170 | SNORD21 | 6083 | Homo sapiens small nucleolar RNA, C/D box 21 |
| | | | (SNORD21), non-coding RNA. |
| 7200035 | DLEC1 | 9940 | Homo sapiens deleted in lung and esophageal cancer 1 |
| | | | (DLEC1), transcript variant DLEC1-S3, mRNA. |
| 7330671 | APOBEC3A | 200315 | Homo sapiens apolipoprotein B mRNA editing enzyme, |
| | | | catalytic polypeptide-like 3A (APOBEC3A), mRNA. |
| 3360343 | RSAD2 | 91543 | Homo sapiens radical S-adenosyl methionine domain |
| | | | containing 2 (RSAD2), mRNA. |
| 5310437 | MYL9 | 10398 | Homo sapiens myosin, light chain 9, regulatory |
| | | | (MYL9), transcript variant 1, mRNA. |
| 5340468 | ITGA2B | 3674 | Homo sapiens integrin, alpha 2b (platelet glycoprotein |
| | | | IIb of IIb/IIIa complex, antigen CD41B) (ITGA2B), |
| | | | mRNA. |
| 1070487 | KLRC3 | 3823 | Homo sapiens killer cell lectin-like receptor subfamily |
| | | | C, member 3 (KLRC3), transcript variant 1, mRNA. |
| 1010246 | IFI6 | 2537 | Homo sapiens interferon, alpha-inducible protein 6 |
| | | | (IFI6), transcript variant 2, mRNA. |
| 1090390 | OAS1 | 4938 | Homo sapiens 2′,5′-oligoadenylate synthetase 1, |
| | | | 40/46 kDa (OAS1), transcript variant 3, mRNA. |
| 7040035 | OAS1 | 4938 | Homo sapiens 2′,5′-oligoadenylate synthetase 1, |
| | | | 40/46 kDa (OAS1), transcript variant 3, mRNA. |
| 990131 | GUCY1A3 | 2982 | Homo sapiens guanylate cyclase 1, soluble, alpha 3 |
| | | | (GUCY1A3), mRNA. |
| 5910632 | SMARCD3 | 6604 | Homo sapiens SWI/SNF related, matrix associated, |
| | | | actin dependent regulator of chromatin, subfamily d, |
| | | | member 3 (SMARCD3), transcript variant 2, mRNA. |
| 6770497 | PLAG1 | 5324 | Homo sapiens pleiomorphic adenoma gene 1 (PLAG1), |
| | | | mRNA. |
| 4180301 | ZNF365 | 22891 | Homo sapiens zinc finger protein 365 (ZNF365), |
| | | | transcript variant A, mRNA. |
| 1820114 | LILRB4 | 11006 | Homo sapiens leukocyte immunoglobulin-like receptor, |
| | | | subfamily B (with TM and ITIM domains), member 4 |
| | | | (LILRB4), transcript variant 2, mRNA. |
| 1240358 | RAB20 | 55647 | Homo sapiens RAB20, member RAS oncogene family |
| | | | (RAB20), mRNA. |
| 1990300 | SOCS1 | 8651 | Homo sapiens suppressor of cytokine signaling 1 |
| | | | (SOCS1), mRNA. |
| 6180491 | ADAM28 | 10863 | Homo sapiens ADAM metallopeptidase domain 28 |
| | | | (ADAM28), transcript variant 3, mRNA. |
| 630315 | DHRS9 | 10170 | Homo sapiens dehydrogenase/reductase (SDR family) |
| | | | member 9 (DHRS9), transcript variant 1, mRNA. |
| 6840600 | GLT25D2 | 23127 | Homo sapiens glycosyltransferase 25 domain |
| | | | containing 2 (GLT25D2), mRNA. |
| 3060044 | LOC642852 | 642852 | PREDICTED: Homo sapiens hypothetical LOC642852 |
| | | | (LOC642852), mRNA. |
| 6760286 | | | RST24587 Athersys RAGE Library Homo sapiens |
| | | | cDNA, mRNA sequence |
| 3130220 | TMEM158 | 25907 | Homo sapiens transmembrane protein 158 |
| | | | (TMEM158), mRNA. |
| 520035 | GTPBP3 | 84705 | Homo sapiens GTP binding protein 3 (mitochondrial) |
| | | | (GTPBP3), nuclear gene encoding mitochondrial |
| | | | protein, transcript variant IV, mRNA. |
| 3290292 | LAP3 | 51056 | Homo sapiens leucine aminopeptidase 3 (LAP3), |
| | | | mRNA. |
| 4850592 | P2RY14 | 9934 | Homo sapiens purinergic receptor P2Y, G-protein |
| | | | coupled, 14 (P2RY14), transcript variant 2, mRNA. |
| 1660504 | ZNF181 | 339318 | Homo sapiens zinc finger protein 181 (ZNF181), |
| | | | mRNA. |
| 6510170 | IFIT3 | 3437 | Homo sapiens interferon-induced protein with |
| | | | tetratricopeptide repeats 3 (IFIT3), mRNA. |
| 1500280 | IFIT3 | 3437 | Homo sapiens interferon-induced protein with |
| | | | tetratricopeptide repeats 3 (IFIT3), mRNA. |
| 1980671 | RIC8B | 55188 | Homo sapiens resistance to inhibitors of cholinesterase |
| | | | 8 homolog B (C. elegans) (RIC8B), mRNA. |
| 2360164 | KLRC2 | 3822 | Homo sapiens killer cell lectin-like receptor subfamily |
| | | | C, member 2 (KLRC2), mRNA. |
| 4150270 | ANKRD22 | 118932 | Homo sapiens ankyrin repeat domain 22 (ANKRD22), |
| | | | mRNA. |
| 6420008 | PROS1 | 5627 | Homo sapiens protein S (alpha) (PROS1), mRNA. |
| 6650735 | | | Homo sapiens T cell receptor alpha locus, mRNA |
| | | | (cDNA clone MGC: 23964 IMAGE: 4687209), complete |
| | | | cds |
| 7610113 | SLC26A8 | 116369 | Homo sapiens solute carrier family 26, member 8 |
| | | | (SLC26A8), transcript variant 2, mRNA. |
| 5360626 | SLC26A8 | 116369 | Homo sapiens solute carrier family 26, member 8 |
| | | | (SLC26A8), transcript variant 2, mRNA. |
| 6580626 | CXorf57 | 55086 | Homo sapiens chromosome X open reading frame 57 |
| | | | (CXorf57), mRNA. |
| 620484 | TNFRSF13B | 23495 | Homo sapiens tumor necrosis factor receptor |
| | | | superfamily, member 13B (TNFRSF13B), mRNA. |
| 4540239 | DEFA1 | 1667 | Homo sapiens defensin, alpha 1 (DEFA1), mRNA. |
| 290739 | DNASE1L3 | 1776 | Homo sapiens deoxyribonuclease I-like 3 |
| | | | (DNASE1L3), mRNA. |
| 6420121 | | | tx34g11.x1 NCI_CGAP_Lu24 Homo sapiens cDNA |
| | | | clone IMAGE: 2271524 3, mRNA sequence |
| 6960687 | DUSP2 | 1844 | Homo sapiens dual specificity phosphatase 2 (DUSP2), |
| | | | mRNA. |
| 6980133 | | | HUMGS0004661 Human adult (K. Okubo) Homo |
| | | | sapiens cDNA 3, mRNA sequence |
| 5700086 | CENPK | 64105 | Homo sapiens centromere protein K (CENPK), mRNA. |
| 6590646 | FAM26F | 441168 | Homo sapiens family with sequence similarity 26, |
| | | | member F (FAM26F), mRNA. |
| 3780193 | FCRLA | 84824 | Homo sapiens Fc receptor-like A (FCRLA), mRNA. |
| 430402 | ABCA3 | 21 | Homo sapiens ATP-binding cassette, sub-family A |
| | | | (ABC1), member 3 (ABCA3), mRNA. |
| 2370639 | C6orf150 | 115004 | Homo sapiens chromosome 6 open reading frame 150 |
| | | | (C6orf150), mRNA. |
| 4900239 | CD274 | 29126 | Homo sapiens CD274 molecule (CD274), mRNA. |
| 6650242 | IFITM3 | 10410 | Homo sapiens interferon induced transmembrane |
| | | | protein 3 (1-8 U) (IFITM3), mRNA. |
| 1820750 | STAT1 | 6772 | Homo sapiens signal transducer and activator of |
| | | | transcription 1, 91 kDa (STAT1), transcript variant |
| | | | alpha, mRNA. |
| 5820064 | MYST4 | 23522 | Homo sapiens MYST histone acetyltransferase |
| | | | (monocytic leukemia) 4 (MYST4), mRNA. |
| 7210356 | | | CR992331 RZPD no. 9016 Homo sapiens cDNA clone |
| | | | RZPDp9016A0141 5, mRNA sequence |
| 6860437 | SLC25A26 | 115286 | Homo sapiens solute carrier family 25, member 26 |
| | | | (SLC25A26), nuclear gene encoding mitochondrial |
| | | | protein, mRNA. |
| 3870338 | IFI44L | 10964 | Homo sapiens interferon-induced protein 44-like |
| | | | (IFI44L), mRNA. |
| 2340577 | AQP10 | 89872 | Homo sapiens aquaporin 10 (AQP10), mRNA. |
| 3290411 | KIAA1804 | 84451 | Homo sapiens mixed lineage kinase 4 (KIAA1804), |
| | | | mRNA. |
| 7510537 | SCO2 | 9997 | Homo sapiens SCO cytochrome oxidase deficient |
| | | | homolog 2 (yeast) (SCO2), nuclear gene encoding |
| | | | mitochondrial protein, mRNA. |
| 3930128 | | | BX110640 Soares_testis_NHT Homo sapiens cDNA |
| | | | clone IMAGp998B094156, mRNA sequence |
| 7400152 | P2RY10 | 27334 | Homo sapiens purinergic receptor P2Y, G-protein |
| | | | coupled, 10 (P2RY10), transcript variant 1, mRNA. |
| 2970747 | DEFA3 | 1668 | Homo sapiens defensin, alpha 3, neutrophil-specific |
| | | | (DEFA3), mRNA. |
| 6760593 | OSBPL10 | 114884 | Homo sapiens oxysterol binding protein-like 10 |
| | | | (OSBPL10), mRNA. |
| 3140487 | BMX | 660 | Homo sapiens BMX non-receptor tyrosine kinase |
| | | | (BMX), mRNA. |
| 520086 | FCGR1A | 2209 | Homo sapiens Fc fragment of IgG, high affinity Ia, |
| | | | receptor (CD64) (FCGR1A), mRNA. |
| 2570300 | IFI44 | 10561 | Homo sapiens interferon-induced protein 44 (IFI44), |
| | | | mRNA. |
| 6280543 | OASL | 8638 | Homo sapiens 2′-5′-oligoadenylate synthetase-like |
| | | | (OASL), transcript variant 1, mRNA. |
| 2450707 | SIGLEC16 | 400709 | Homo sapiens sialic acid binding Ig-like lectin 16 |
| | | | (gene/pseudogene) (SIGLEC16), non-coding RNA. |
| 5570039 | LOC728744 | 728744 | PREDICTED: Homo sapiens hypothetical LOC728744 |
| | | | (LOC728744), mRNA. |
| 3710068 | WARS | 7453 | Homo sapiens tryptophanyl-tRNA synthetase (WARS), |
| | | | transcript variant 2, mRNA. |
| 3180528 | MMP9 | 4318 | Homo sapiens matrix metallopeptidase 9 (gelatinase B, |
| | | | 92 kDa gelatinase, 92 kDa type IV collagenase) |
| | | | (MMP9), mRNA. |
| 430255 | LOC647606 | 647606 | PREDICTED: Homo sapiens hypothetical protein |
| | | | LOC647606 (LOC647606), mRNA. |
| 460463 | SMARCD3 | 6604 | Homo sapiens SWI/SNF related, matrix associated, |
| | | | actin dependent regulator of chromatin, subfamily d, |
| | | | member 3 (SMARCD3), transcript variant 1, mRNA. |
| 6860717 | HOXC4 | 3221 | Homo sapiens homeobox C4 (HOXC4), transcript |
| | | | variant 1, mRNA. |
| 3800576 | ASGR2 | 433 | Homo sapiens asialoglycoprotein receptor 2 (ASGR2), |
| | | | transcript variant 3, mRNA. |
| 4280273 | GM2A | 2760 | Homo sapiens GM2 ganglioside activator (GM2A), |
| | | | mRNA. |
| 1980524 | GBP4 | 115361 | Homo sapiens guanylate binding protein 4 (GBP4), |
| | | | mRNA. |
| 4560561 | BACH2 | 60468 | Homo sapiens BTB and CNC homology 1, basic |
| | | | leucine zipper transcription factor 2 (BACH2), mRNA. |
| 6110575 | FAIM | 55179 | Homo sapiens Fas apoptotic inhibitory molecule |
| | | | (FAIM), transcript variant 1, mRNA. |
| 290437 | HRK | 8739 | Homo sapiens harakiri, BCL2 interacting protein |
| | | | (contains only BH3 domain) (HRK), mRNA. |
| 5670100 | TCN2 | 6948 | Homo sapiens transcobalamin II; macrocytic anemia |
| | | | (TCN2), mRNA. |
| 1440341 | C1QC | 714 | Homo sapiens complement component 1, q |
| | | | subcomponent, C chain (C1QC), mRNA. |
| 270240 | SLC26A8 | 116369 | Homo sapiens solute carrier family 26, member 8 |
| | | | (SLC26A8), transcript variant 1, mRNA. |
| 4860224 | WARS | 7453 | Homo sapiens tryptophanyl-tRNA synthetase (WARS), |
| | | | transcript variant 1, mRNA. |
| 3290072 | | | BX116726 NCI_CGAP_Pr28 Homo sapiens cDNA |
| | | | clone IMAGp998J065569, mRNA sequence |
| 4220187 | DYSF | 8291 | Homo sapiens dysferlin, limb girdle muscular |
| | | | dystrophy 2B (autosomal recessive) (DYSF), mRNA. |
| 2340441 | SLC22A18AS | 5003 | Homo sapiens solute carrier family 22 (organic cation |
| | | | transporter), member 18 antisense (SLC22A18AS), |
| | | | mRNA. |
| 520408 | IFIT3 | 3437 | Homo sapiens interferon-induced protein with |
| | | | tetratricopeptide repeats 3 (IFIT3), mRNA. |
| 3520601 | MPO | 4353 | Homo sapiens myeloperoxidase (MPO), nuclear gene |
| | | | encoding mitochondrial protein, mRNA. |
| 4010184 | C8orf13 | 83648 | Homo sapiens chromosome 8 open reading frame 13 |
| | | | (C8orf13), mRNA. |
| 1410221 | S100A12 | 6283 | Homo sapiens S100 calcium binding protein A12 |
| | | | (S100A12), mRNA. |
| 6270553 | CXCL10 | 3627 | Homo sapiens chemokine (C-X-C motif) ligand 10 |
| | | | (CXCL10), mRNA. |
| 4120424 | COCH | 1690 | Homo sapiens coagulation factor C homolog, cochlin |
| | | | (Limulus polyphemus) (COCH), mRNA. |
| 1470176 | SYT17 | 51760 | Homo sapiens synaptotagmin XVII (SYT17), mRNA. |
| 6290612 | TMEM107 | 84314 | Homo sapiens transmembrane protein 107 |
| | | | (TMEM107), transcript variant 1, mRNA. |
| 3940703 | RORA | 6095 | Homo sapiens RAR-related orphan receptor A (RORA), |
| | | | transcript variant 3, mRNA. |
| 4670458 | Sep-04 | 5414 | Homo sapiens septin 4 (SEPT4), transcript variant 2, |
| | | | mRNA. |
| 6200156 | | | BX107738 Soares_testis_NHT Homo sapiens cDNA |
| | | | clone IMAGp998F181825, mRNA sequence |
| 1450427 | RTP4 | 64108 | Homo sapiens receptor (chemosensory) transporter |
| | | | protein 4 (RTP4), mRNA. |
| 4210039 | ABCB1 | 5243 | Homo sapiens ATP-binding cassette, sub-family B |
| | | | (MDR/TAP), member 1 (ABCB1), mRNA. |
| 3780047 | GBP6 | 163351 | Homo sapiens guanylate binding protein family, |
| | | | member 6 (GBP6), mRNA. |
| 7380400 | GBP6 | 163351 | Homo sapiens guanylate binding protein family, |
| | | | member 6 (GBP6), mRNA. |
| 5690463 | C11orf80 | 79703 | Homo sapiens chromosome 11 open reading frame 80 |
| | | | (C11orf80), mRNA. |
| 3180474 | | | DKFZp686H1820_r1 686 (synonym: hlcc3) Homo |
| | | | sapiens cDNA clone DKFZp686H1820 5, mRNA |
| | | | sequence |
| 4060008 | CAMK2N1 | 55450 | Homo sapiens calcium/calmodulin-dependent protein |
| | | | kinase II inhibitor 1 (CAMK2N1), mRNA. |
| 5490403 | CD1E | 913 | Homo sapiens CD1e molecule (CD1E), transcript |
| | | | variant 5, mRNA. |
| 7160468 | DHRS9 | 10170 | Homo sapiens dehydrogenase/reductase (SDR family) |
| | | | member 9 (DHRS9), transcript variant 1, mRNA. |
| 5570343 | TWISTNB | 221830 | Homo sapiens TWIST neighbor (TWISTNB), mRNA. |
| 50324 | ITPRIPL1 | 150771 | Homo sapiens inositol 1,4,5-triphosphate receptor |
| | | | interacting protein-like 1 (ITPRIPL1), transcript variant |
| | | | 2, mRNA. |
| 2140242 | TNFAIP6 | 7130 | Homo sapiens tumor necrosis factor, alpha-induced |
| | | | protein 6 (TNFAIP6), mRNA. |

TABLE 5
List of the 86 genes

| ProbeID | Symbol | Entrez_Gene_ID | Definition |
|---|---|---|---|
| 5820753 | RBCK1 | 10616 | Homo sapiens RanBP-type and C3HC4-type zinc finger |
| | | | containing 1 (RBCK1), transcript variant 1, mRNA. |
| 1580427 | THEM2 | 55856 | Homo sapiens thioesterase superfamily member 2 (THEM2), |
| | | | mRNA. |
| 1030201 | WDR33 | 55339 | Homo sapiens WD repeat domain 33 (WDR33), transcript |
| | | | variant 3, mRNA. |
| 380102 | TMEM51 | 55092 | Homo sapiens transmembrane protein 51 (TMEM51), mRNA. |
| 4860438 | DMXL2 | 23312 | Homo sapiens Dmx-like 2 (DMXL2), mRNA. |
| 6380338 | POLB | 5423 | Homo sapiens polymerase (DNA directed), beta (POLB), |
| | | | mRNA. |
| 3170273 | FER1L3 | 26509 | Homo sapiens fer-1-like 3, myoferlin (C. elegans) (FER1L3), |
| | | | transcript variant 2, mRNA. |
| 610598 | CHI3L2 | 1117 | Homo sapiens chitinase 3-like 2 (CHI3L2), transcript variant |
| | | | 1, mRNA. |
| 7560706 | C9orf109 | 286333 | PREDICTED: Homo sapiens chromosome 9 open reading |
| | | | frame 109 (C9orf109), mRNA. |
| 4040450 | VCPIP1 | 80124 | Homo sapiens valosin containing protein (p97)/p47 complex |
| | | | interacting protein 1 (VCPIP1), mRNA. |
| 5270403 | | | Homo sapiens cDNA FLJ20012 fis, clone ADKA03438 |
| 5900564 | SLC16A6 | 9120 | Homo sapiens solute carrier family 16, member 6 |
| | | | (monocarboxylic acid transporter 7) (SLC16A6), mRNA. |
| 1570669 | LACTB | 114294 | Homo sapiens lactamase, beta (LACTB), nuclear gene |
| | | | encoding mitochondrial protein, transcript variant 1, mRNA. |
| 1510088 | ATP1B3 | 483 | PREDICTED: Homo sapiens ATPase, Na+/K+ transporting, |
| | | | beta 3 polypeptide, transcript variant 2 (ATP1B3), mRNA. |
| 6760450 | CAPS | 828 | Homo sapiens calcyphosine (CAPS), transcript variant 2, |
| | | | mRNA. |
| 5900692 | GSTK1 | 373156 | Homo sapiens glutathione S-transferase kappa 1 (GSTK1), |
| | | | mRNA. |
| 7380100 | MCL1 | 4170 | Homo sapiens myeloid cell leukemia sequence 1 (BCL2- |
| | | | related) (MCL1), transcript variant 1, mRNA. |
| 2360537 | NFKBIB | 4793 | Homo sapiens nuclear factor of kappa light polypeptide gene |
| | | | enhancer in B-cells inhibitor, beta (NFKBIB), transcript |
| | | | variant 1, mRNA. |
| 1510538 | EPB41L3 | 23136 | Homo sapiens erythrocyte membrane protein band 4.1-like 3 |
| | | | (EPB41L3), mRNA. |
| 240392 | | | Homo sapiens cDNA FLJ34585 fis, clone KIDNE2008758 |
| 5220070 | HLA-F | 3134 | Homo sapiens major histocompatibility complex, class I, F |
| | | | (HLA-F), transcript variant 1, mRNA. |
| 2570328 | TR1B2 | 28951 | Homo sapiens tribbles homolog 2 (Drosophila) (TRIB2), |
| | | | mRNA. |
| 6550703 | WSB2 | 55884 | Homo sapiens WD repeat and SOCS box-containing 2 |
| | | | (WSB2), mRNA. |
| 450468 | MRPL44 | 65080 | Homo sapiens mitochondrial ribosomal protein L44 |
| | | | (MRPL44), nuclear gene encoding mitochondrial protein, |
| | | | mRNA. |
| 4290209 | TMC6 | 11322 | Homo sapiens transmembrane channel-like 6 (TMC6), |
| | | | mRNA. |
| 2760730 | LOC644086 | 644086 | PREDICTED: Homo sapiens hypothetical protein |
| | | | LOC644086, transcript variant 1 (LOC644086), mRNA. |
| 2490240 | PTPRE | 5791 | Homo sapiens protein tyrosine phosphatase, receptor type, E |
| | | | (PTPRE), transcript variant 2, mRNA. |
| 1770152 | MS4A6A | 64231 | Homo sapiens membrane-spanning 4-domains, subfamily A, |
| | | | member 6A (MS4A6A), transcript variant 3, mRNA. |
| 6290452 | NAT1 | 9 | PREDICTED: Homo sapiens N-acetyltransferase 1 (arylamine |
| | | | N-acetyltransferase) (NAT1), mRNA. |
| 110161 | EFR3A | 23167 | Homo sapiens EFR3 homolog A (S. cerevisiae) (EFR3A), |
| | | | mRNA. |
| 4590228 | GLRX | 2745 | Homo sapiens glutaredoxin (thioltransferase) (GLRX), |
| | | | mRNA. |
| 4810079 | KIAA1632 | 57724 | Homo sapiens KIAA1632 (KIAA1632), mRNA. |
| 4890592 | PDE7A | 5150 | Homo sapiens phosphodiesterase 7A (PDE7A), transcript |
| | | | variant 2, mRNA. |
| 4730343 | ATP6V0E1 | 8992 | Homo sapiens ATPase, H+ transporting, lysosomal 9 kDa, V0 |
| | | | subunit e1 (ATP6V0E1), mRNA. |
| 2320110 | ATP6V0E1 | 8992 | Homo sapiens ATPase, H+ transporting, lysosomal 9 kDa, V0 |
| | | | subunit e1 (ATP6V0E1), mRNA. |
| 6100136 | CASP4 | 837 | Homo sapiens caspase 4, apoptosis-related cysteine peptidase |
| | | | (CASP4), transcript variant gamma, mRNA. |
| 1660300 | PRCP | 5547 | Homo sapiens prolylcarboxypeptidase (angiotensinase C) |
| | | | (PRCP), transcript variant 2, mRNA. |
| 110064 | RNASEL | 6041 | Homo sapiens ribonuclease L (2′,5′-oligoisoadenylate |
| | | | synthetase-dependent) (RNASEL), mRNA. |
| 2900524 | CCR2 | 1231 | Homo sapiens chemokine (C-C motif) receptor 2 (CCR2), |
| | | | transcript variant B, mRNA. |
| 7200753 | TLR7 | 51284 | Homo sapiens toll-like receptor 7 (TLR7), mRNA. |
| 2060600 | RBMS1 | 5937 | Homo sapiens RNA binding motif, single stranded interacting |
| | | | protein 1 (RBMS1), transcript variant 3, mRNA. |
| 2000445 | PSMB10 | 5699 | Homo sapiens proteasome (prosome, macropain) subunit, beta |
| | | | type, 10 (PSMB10), mRNA. |
| 770538 | LYSMD2 | 256586 | Homo sapiens LysM, putative peptidoglycan-binding, domain |
| | | | containing 2 (LYSMD2), mRNA. |
| 6580717 | ZMYND15 | 84225 | Homo sapiens zinc finger, MYND-type containing 15 |
| | | | (ZMYND15), mRNA. |
| 1230278 | LOC440348 | 440348 | Homo sapiens similar to nuclear pore complex interacting |
| | | | protein (LOC440348), mRNA. |
| 4040711 | PSMA4 | 5685 | Homo sapiens proteasome (prosome, macropain) subunit, |
| | | | alpha type, 4 (PSMA4), mRNA. |
| 3420543 | PSMA4 | 5685 | Homo sapiens proteasome (prosome, macropain) subunit, |
| | | | alpha type, 4 (PSMA4), mRNA. |
| 3710332 | PSMB3 | 5691 | Homo sapiens proteasome (prosome, macropain) subunit, beta |
| | | | type, 3 (PSMB3), mRNA. |
| 3180554 | PIGU | 128869 | Homo sapiens phosphatidylinositol glycan anchor |
| | | | biosynthesis, class U (PIGU), mRNA. |
| 1990041 | LRRC37A4 | 55073 | Homo sapiens leucine rich repeat containing 37, member A4 |
| | | | (pseudogene) (LRRC37A4), non-coding RNA. XM_934274 |
| | | | XM_941652 XM_945358 XM_945361 XM_945364 |
| 630204 | RAC1 | 5879 | Homo sapiens ras-related C3 botulinum toxin substrate 1 (rho |
| | | | family, small GTP binding protein Rac1) (RAC1), transcript |
| | | | variant Rac1, mRNA. |
| 1030010 | POMP | 51371 | Homo sapiens proteasome maturation protein (POMP), |
| | | | mRNA. |
| 2370358 | TYROBP | 7305 | Homo sapiens TYRO protein tyrosine kinase binding protein |
| | | | (TYROBP), transcript variant 1, mRNA. |
| 290022 | Sep-01 | 1731 | PREDICTED: Homo sapiens septin 1, transcript variant 3 |
| | | | (SEPT1), mRNA. |
| 6840577 | KPNB1 | 3837 | Homo sapiens karyopherin (importin) beta 1 (KPNB1), |
| | | | mRNA. |
| 7650343 | USP47 | 55031 | Homo sapiens ubiquitin specific peptidase 47 (USP47), |
| | | | mRNA. |
| 1010195 | DBI | 1622 | Homo sapiens diazepam binding inhibitor (GABA receptor |
| | | | modulator, acyl-Coenzyme A binding protein) (DBI), |
| | | | transcript variant 2, mRNA. |
| 520010 | LOC284701 | 284701 | PREDICTED: Homo sapiens hypothetical protein |
| | | | LOC284701, transcript variant 2 (LOC284701), mRNA. |
| 6020669 | LOC391811 | 391811 | PREDICTED: Homo sapiens similar to DNA polymerase |
| | | | delta subunit 2 (DNA polymerase delta subunit p50) |
| | | | (LOC391811), mRNA. |
| 4760537 | C20orf24 | 55969 | Homo sapiens chromosome 20 open reading frame 24 |
| | | | (C20orf24), transcript variant 2, mRNA. |
| 830035 | ATG3 | 64422 | Homo sapiens ATG3 autophagy related 3 homolog |
| | | | (S. cerevisiae) (ATG3), mRNA. |
| 4890255 | AGMAT | 79814 | Homo sapiens agmatine ureohydrolase (agmatinase) |
| | | | (AGMAT), mRNA. |
| 5390246 | CCR7 | 1236 | Homo sapiens chemokine (C-C motif) receptor 7 (CCR7), |
| | | | mRNA. |
| 2120224 | ANKRD13A | 88455 | Homo sapiens ankyrin repeat domain 13A (ANKRD13A), |
| | | | mRNA. |
| 3390221 | EIF4E3 | 317649 | Homo sapiens eukaryotic translation initiation factor 4E |
| | | | family member 3 (EIF4E3), mRNA. |
| 6380364 | SLC7A6 | 9057 | Homo sapiens solute carrier family 7 (cationic amino acid |
| | | | transporter, y+ system), member 6 (SLC7A6), transcript |
| | | | variant 2, mRNA. |
| 520484 | USP15 | 9958 | Homo sapiens ubiquitin specific peptidase 15 (USP15), |
| | | | mRNA. |
| 6040338 | LOC149448 | 149448 | PREDICTED: Homo sapiens hypothetical protein |
| | | | LOC149448 (LOC149448), mRNA. |
| 1440180 | C20orf24 | 55969 | Homo sapiens chromosome 20 open reading frame 24 |
| | | | (C20orf24), transcript variant 1, mRNA. |
| 540075 | NPC2 | 10577 | Homo sapiens Niemann-Pick disease, type C2 (NPC2), |
| | | | mRNA. |
| 2000110 | BRSK1 | 84446 | Homo sapiens BR serine/threonine kinase 1 (BRSK1), |
| | | | mRNA. |
| 3610280 | C9orf127 | 51754 | Homo sapiens chromosome 9 open reading frame 127 |
| | | | (C9orf127), mRNA. |
| 1090291 | DEPDC5 | 9681 | Homo sapiens DEP domain containing 5 (DEPDC5), |
| | | | transcript variant 1, mRNA. |
| 4560047 | CD74 | 972 | Homo sapiens CD74 molecule, major histocompatibility |
| | | | complex, class II invariant chain (CD74), transcript variant 1, |
| | | | mRNA. |
| 6620689 | MTHFD2 | 10797 | Homo sapiens methylenetetrahydrofolate dehydrogenase |
| | | | (NADP+ dependent) 2, methenyltetrahydrofolate |
| | | | cyclohydrolase (MTHFD2), nuclear gene encoding |
| | | | mitochondrial protein, transcript variant 2, mRNA. |
| 870632 | | | Homo sapiens clone 23700 mRNA sequence |
| 5820646 | CALCOCO2 | 10241 | Homo sapiens calcium binding and coiled-coil domain 2 |
| | | | (CALCOCO2), mRNA. |
| 1230068 | PPP1R3D | 5509 | Homo sapiens protein phosphatase 1, regulatory (inhibitor) |
| | | | subunit 3D (PPP1R3D), mRNA. |
| 6900301 | C4orf34 | 201895 | Homo sapiens chromosome 4 open reading frame 34 |
| | | | (C4orf34), mRNA. |
| 5700110 | | | qw67g08.x1 NCI_CGAP_Ov33 Homo sapiens cDNA clone |
| | | | IMAGE: 1996190 3, mRNA sequence |
| 6520725 | TNFRSF14 | 8764 | Homo sapiens tumor necrosis factor receptor superfamily, |
| | | | member 14 (herpesvirus entry mediator) (TNFRSF14), |
| | | | mRNA. |
| 2360035 | FCRL3 | 115352 | Homo sapiens Fc receptor-like 3 (FCRL3), transcript variant |
| | | | 2, mRNA. |
| 7320348 | CLEC1A | 51267 | Homo sapiens C-type lectin domain family 1, member A |
| | | | (CLEC1A), mRNA. |
| 6370133 | SEC23B | 10483 | Homo sapiens Sec23 homolog B (S. cerevisiae) (SEC23B), |
| | | | transcript variant 2, mRNA. |
| 2760112 | P2RY5 | 10161 | Homo sapiens purinergic receptor P2Y, G-protein coupled, 5 |
| | | | (P2RY5), mRNA. |
| 4490079 | DHRS9 | 10170 | Homo sapiens dehydrogenase/reductase (SDR family) |
| | | | member 9 (DHRS9), transcript variant 1, mRNA. |

TABLE 6
List of the 393 genes

| ProbeID | Symbol | Entrez_Gene_ID | Definition |
|---|---|---|---|
| 2190349 | KCNJ15 | 3772 | Homo sapiens potassium inwardly-rectifying channel, |
| | | | subfamily J, member 15 (KCNJ15), transcript variant 3, |
| | | | mRNA. |
| 5570139 | QPCT | 25797 | Homo sapiens glutaminyl-peptide cyclotransferase (QPCT), |
| | | | mRNA. |
| 5700725 | EPSTI1 | 94240 | Homo sapiens epithelial stromal interaction 1 (breast) |
| | | | (EPSTI1), transcript variant 2, mRNA. |
| 5220538 | WDFY3 | 23001 | Homo sapiens WD repeat and FYVE domain containing 3 |
| | | | (WDFY3), transcript variant 2, mRNA. |
| 2070646 | GPR84 | 53831 | Homo sapiens G protein-coupled receptor 84 (GPR84), |
| | | | mRNA. |
| 520162 | HPSE | 10855 | Homo sapiens heparanase (HPSE), mRNA. |
| 3850440 | FCER1G | 2207 | Homo sapiens Fc fragment of IgE, high affinity I, receptor |
| | | | for; gamma polypeptide (FCER1G), mRNA. |
| 130181 | ANKRD22 | 118932 | Homo sapiens ankyrin repeat |
| | | | domain 22 (ANKRD22), mRNA. |
| 620136 | LILRA5 | 353514 | Homo sapiens leukocyte immunoglobulin-like receptor, |
| | | | subfamily A (with TM domain), member 5 (LILRA5), |
| | | | transcript variant 3, mRNA. |
| 160370 | TPM2 | 7169 | Homo sapiens tropomyosin 2 (beta) (TPM2), transcript |
| | | | variant 2, mRNA. |
| 4290368 | PSTPIP2 | 9050 | Homo sapiens proline-serine-threonine phosphatase |
| | | | interacting protein 2 (PSTPIP2), mRNA. |
| 2360348 | CMPK2 | 129607 | Homo sapiens cytidine monophosphate (UMP-CMP) kinase |
| | | | 2, mitochondrial (CMPK2), nuclear gene encoding |
| | | | mitochondrial protein, mRNA. |
| 2630195 | VAMP5 | 10791 | Homo sapiens vesicle-associated membrane protein 5 |
| | | | (myobrevin) (VAMP5), mRNA. |
| 6620209 | FCGR1B | 2210 | Homo sapiens Fc fragment of IgG, high affinity Ib, receptor |
| | | | (CD64) (FCGR1B), transcript variant 2, mRNA. |
| 130609 | FCGBP | 8857 | Homo sapiens Fc fragment of IgG binding protein (FCGBP), |
| | | | mRNA. |
| 7210035 | SNORD13 | 692084 | Homo sapiens small nucleolar RNA, C/D box 13 |
| | | | (SNORD13), non-coding RNA. |
| 20048 | HIST2H2BF | 440689 | Homo sapiens histone cluster 2, H2bf (HIST2H2BF), |
| | | | mRNA. |
| 3990010 | | | AGENCOURT_7914287 NIH_MGC_71 Homo sapiens |
| | | | cDNA clone IMAGE: 6156595 5, mRNA sequence |
| 1510364 | GBP5 | 115362 | Homo sapiens guanylate binding protein 5 (GBP5), mRNA. |
| 1340241 | C5 | 727 | Homo sapiens complement component 5 (C5), mRNA. |
| 670202 | SESTD1 | 91404 | Homo sapiens SEC14 and spectrin domains 1 (SESTD1), |
| | | | mRNA. |
| 6370768 | ETV7 | 51513 | Homo sapiens ets variant 7 (ETV7), mRNA. |
| 7160274 | TXNDC12 | 51060 | Homo sapiens thioredoxin domain containing 12 |
| | | | (endoplasmic reticulum) (TXNDC12), mRNA. |
| 6400148 | SELM | 140606 | Homo sapiens selenoprotein M (SELM), mRNA. |
| 3120440 | | | full-length cDNA clone CS0DI056YK21 of Placenta Cot |
| | | | 25-normalized of Homo sapiens (human) |
| 6100474 | EVI2A | 2123 | Homo sapiens ecotropic viral integration site 2A (EVI2A), |
| | | | transcript variant 1, mRNA. |
| 4280632 | GAS6 | 2621 | Homo sapiens growth arrest-specific 6 (GAS6), mRNA. |
| 70730 | GAS6 | 2621 | Homo sapiens growth arrest-specific 6 (GAS6), mRNA. |
| 1470091 | IL15 | 3600 | Homo sapiens interleukin 15 (IL15), transcript variant 1, |
| | | | mRNA. |
| 2490315 | | | Homo sapiens cDNA: FLJ23098 fis, clone LNG07440 |
| 110639 | MAPK14 | 1432 | Homo sapiens mitogen-activated protein kinase 14 |
| | | | (MAPK14), transcript variant 2, mRNA. |
| 5050041 | GPR109B | 8843 | Homo sapiens G protein-coupled receptor 109B |
| | | | (GPR109B), mRNA. |
| 6560156 | DUSP3 | 1845 | Homo sapiens dual specificity phosphatase 3 (vaccinia virus |
| | | | phosphatase VH1-related) (DUSP3), mRNA. |
| 380102 | TMEM51 | 55092 | Homo sapiens transmembrane protein 51 (TMEM51), |
| | | | mRNA. |
| 3520327 | LOC642161 | 642161 | PREDICTED: Homo sapiens similar to T-cell receptor beta |
| | | | chain V region CTL-L17 precursor (LOC642161), mRNA. |
| 2760500 | CD38 | 952 | Homo sapiens CD38 molecule (CD38), mRNA. |
| 7570324 | ID3 | 3399 | Homo sapiens inhibitor of DNA binding 3, dominant |
| | | | negative helix-loop-helix protein (ID3), mRNA. |
| 3940356 | CR1 | 1378 | Homo sapiens complement component (3b/4b) receptor 1 |
| | | | (Knops blood group) (CR1), transcript variant S, mRNA. |
| 3370053 | | | Homo sapiens T cell receptor beta variable 21-1, mRNA |
| | | | (cDNA clone MGC: 46491 IMAGE: 5225843), complete cds |
| 2030309 | SERPING1 | 710 | Homo sapiens serpin peptidase inhibitor, clade G (C1 |
| | | | inhibitor), member 1 (SERPING1), transcript variant 2, |
| | | | mRNA. |
| 6770707 | SQRDL | 58472 | Homo sapiens sulfide quinone reductase-like (yeast) |
| | | | (SQRDL), mRNA. |
| 3170273 | FER1L3 | 26509 | Homo sapiens fer-1-like 3, myoferlin (C. elegans) |
| | | | (FER1L3), transcript variant 2, mRNA. |
| 6940164 | LBH | 81606 | PREDICTED: Homo sapiens hypothetical protein |
| | | | DKFZp566J091 (LBH), mRNA. |
| 4230112 | LBH | 81606 | PREDICTED: Homo sapiens hypothetical protein |
| | | | DKFZp566J091 (LBH), mRNA. |
| 5870008 | | | Human mRNA for T-cell specific protein |
| 7400743 | KIAA1618 | 57714 | Homo sapiens KIAA1618 (KIAA1618), mRNA. |
| 110338 | XRN1 | 54464 | Homo sapiens 5′-3′ exoribonuclease 1 (XRN1), transcript |
| | | | variant 2, mRNA. |
| 4120561 | LIMK2 | 3985 | Homo sapiens LIM domain kinase 2 (LIMK2), transcript |
| | | | variant 1, mRNA. |
| 4200719 | LOC730092 | 730092 | Homo sapiens RRN3 RNA polymerase I transcription factor |
| | | | homolog (S. cerevisiae) pseudogene (LOC730092) on |
| | | | chromosome 16. |
| 2680050 | SIRPG | 55423 | Homo sapiens signal-regulatory protein gamma (SIRPG), |
| | | | transcript variant 2, mRNA. |
| 7560520 | | | Homo sapiens cDNA FLJ41813 fis, clone NT2RI2011450 |
| 5270403 | | | Homo sapiens cDNA FLJ20012 fis, clone ADKA03438 |
| 5360156 | IFITM1 | 8519 | Homo sapiens interferon induced transmembrane protein 1 |
| | | | (9-27) (IFITM1), mRNA. |
| 3830228 | GPR109A | 338442 | Homo sapiens G protein-coupled receptor 109A |
| | | | (GPR109A), mRNA. |
| 70431 | LOC650799 | 650799 | PREDICTED: Homo sapiens similar to Ig lambda chain V-I |
| | | | region BL2 precursor (LOC650799), mRNA. |
| 2510220 | IFI35 | 3430 | Homo sapiens interferon-induced protein 35 (IFI35), |
| | | | mRNA. |
| 6400176 | IRF7 | 3665 | Homo sapiens interferon regulatory factor 7 (IRF7), |
| | | | transcript variant b, mRNA. |
| 2570112 | ABLIM1 | 3983 | Homo sapiens actin binding LIM protein 1 (ABLIM1), |
| | | | transcript variant 4, mRNA. |
| 5310445 | KREMEN1 | 83999 | Homo sapiens kringle containing transmembrane protein 1 |
| | | | (KREMEN1), transcript variant 4, mRNA. |
| 3390301 | KREMEN1 | 83999 | Homo sapiens kringle containing transmembrane protein 1 |
| | | | (KREMEN1), transcript variant 4, mRNA. |
| 6840035 | GBP1 | 2633 | Homo sapiens guanylate binding protein 1, interferon- |
| | | | inducible, 67 kDa (GBP1), mRNA. |
| 2190148 | GBP1 | 2633 | Homo sapiens guanylate binding protein 1, interferon- |
| | | | inducible, 67 kDa (GBP1), mRNA. |
| 3870594 | IFI16 | 3428 | Homo sapiens interferon, gamma-inducible protein 16 |
| | | | (IFI16), mRNA. |
| 240053 | GCH1 | 2643 | Homo sapiens GTP cyclohydrolase 1 (GCH1), transcript |
| | | | variant 4, mRNA. |
| 3830349 | IL7R | 3575 | Homo sapiens interleukin 7 receptor (IL7R), mRNA. |
| 1260424 | ADAM7 | 8756 | Homo sapiens ADAM metallopeptidase domain 7 |
| | | | (ADAM7), mRNA. |
| 3800398 | FBXO6 | 26270 | Homo sapiens F-box protein 6 (FBXO6), mRNA. |
| 5900524 | LACTB | 114294 | Homo sapiens lactamase, beta (LACTB), nuclear gene |
| | | | encoding mitochondrial protein, transcript variant 2, mRNA. |
| 5550279 | ENTPD1 | 953 | Homo sapiens ectonucleoside triphosphate |
| | | | diphosphohydrolase 1 (ENTPD1), transcript variant 2, |
| | | | mRNA. |
| 1430711 | KLF12 | 11278 | Homo sapiens Kruppel-like factor 12 (KLF12), mRNA. |
| 6770017 | KLF12 | 11278 | Homo sapiens Kruppel-like factor 12 (KLF12), mRNA. |
| 1570129 | TRAFD1 | 10906 | Homo sapiens TRAF-type zinc finger domain containing 1 |
| | | | (TRAFD1), mRNA. |
| 6200577 | CREB5 | 9586 | Homo sapiens cAMP responsive element binding protein 5 |
| | | | (CREB5), transcript variant 1, mRNA. |
| 4730059 | BATF2 | 116071 | Homo sapiens basic leucine zipper transcription factor, |
| | | | ATF-like 2 (BATF2), mRNA. |
| 1510538 | EPB41L3 | 23136 | Homo sapiens erythrocyte membrane protein band 4.1-like 3 |
| | | | (EPB41L3), mRNA. |
| 2070114 | CLEC4D | 338339 | Homo sapiens C-type lectin domain family 4, member D |
| | | | (CLEC4D), mRNA. |
| 7510647 | CACNA1E | 777 | Homo sapiens calcium channel, voltage-dependent, R type, |
| | | | alpha 1E subunit (CACNA1E), mRNA. |
| 2570328 | TRIB2 | 28951 | Homo sapiens tribbles homolog 2 (Drosophila) (TRIB2), |
| | | | mRNA. |
| 730528 | PLAUR | 5329 | Homo sapiens plasminogen activator, urokinase receptor |
| | | | (PLAUR), transcript variant 1, mRNA. |
| 2970286 | CEACAM1 | 634 | Homo sapiens carcinoembryonic antigen-related cell |
| | | | adhesion molecule 1 (biliary glycoprotein) (CEACAM1), |
| | | | transcript variant 2, mRNA. |
| 5700753 | CEACAM1 | 634 | Homo sapiens carcinoembryonic antigen-related cell |
| | | | adhesion molecule 1 (biliary glycoprotein) (CEACAM1), |
| | | | transcript variant 2, mRNA. |
| 4610180 | PIK3C2B | 5287 | Homo sapiens phosphoinositide-3-kinase, class 2, beta |
| | | | polypeptide (PIK3C2B), mRNA. |
| 3940477 | TAP2 | 6891 | Homo sapiens transporter 2, ATP-binding cassette, sub- |
| | | | family B (MDR/TAP) (TAP2), transcript variant 1, mRNA. |
| 3460053 | SIPA1L2 | 57568 | Homo sapiens signal-induced proliferation-associated 1 like |
| | | | 2(SIPA1L2), mRNA. |
| 1510026 | FLVCR2 | 55640 | Homo sapiens feline leukemia virus subgroup C cellular |
| | | | receptor family, member 2 (FLVCR2), mRNA. |
| 3420593 | LMNB1 | 4001 | Homo sapiens lamin B1 (LMNB1), mRNA. |
| 1940180 | PSMB9 | 5698 | Homo sapiens proteasome (prosome, macropain) subunit, |
| | | | beta type, 9 (large multifunctional peptidase 2) (PSMB9), |
| | | | transcript variant 1, mRNA. |
| 4780128 | ATF3 | 467 | Homo sapiens activating transcription factor 3 (ATF3), |
| | | | transcript variant 4, mRNA. |
| 5050347 | CD5 | 921 | Homo sapiens CD5 molecule (CD5), mRNA. |
| 160019 | SORT1 | 6272 | Homo sapiens sortilin 1 (SORT1), mRNA. |
| 2060440 | MAFB | 9935 | Homo sapiens v-maf musculoaponeurotic fibrosarcoma |
| | | | oncogene homolog B (avian) (MAFB), mRNA. |
| 2570019 | C11orf82 | 220042 | Homo sapiens chromosome 11 open reading frame 82 |
| | | | (C11orf82), mRNA. |
| 6330471 | BLK | 640 | Homo sapiens B lymphoid tyrosine kinase (BLK), mRNA. |
| 5390730 | PIK3IP1 | 113791 | Homo sapiens phosphoinositide-3-kinase interacting protein |
| | | | 1 (PIK3IP1), mRNA. |
| 3440360 | ZNF438 | 220929 | Homo sapiens zinc finger protein 438 (ZNF438), mRNA. |
| 130661 | LOC650795 | 650795 | PREDICTED: Homo sapiens similar to T-cell receptor alpha |
| | | | chain V region PY14 precursor (LOC650795), mRNA. |
| 4260386 | CTSL1 | 1514 | Homo sapiens cathepsin L1 (CTSL1), transcript variant 2, |
| | | | mRNA. |
| 6480059 | ACTA2 | 59 | Homo sapiens actin, alpha 2, smooth muscle, aorta |
| | | | (ACTA2), mRNA. |
| 3130669 | SATB1 | 6304 | Homo sapiens SATB homeobox 1 (SATB1), mRNA. |
| 6100136 | CASP4 | 837 | Homo sapiens caspase 4, apoptosis-related cysteine |
| | | | peptidase (CASP4), transcript variant gamma, mRNA. |
| 6040398 | SLC22A4 | 6583 | Homo sapiens solute carrier family 22 (organic |
| | | | cation/ergothioneine transporter), member 4 (SLC22A4), |
| | | | mRNA. |
| 6420750 | TLR2 | 7097 | Homo sapiens toll-like receptor 2 (TLR2), mRNA. |
| 2760689 | MAGEE1 | 57692 | Homo sapiens melanoma antigen family E, 1 (MAGEE1), |
| | | | mRNA. |
| 3420373 | SOD2 | 6648 | Homo sapiens superoxide dismutase 2, mitochondrial |
| | | | (SOD2), nuclear gene encoding mitochondrial protein, |
| | | | transcript variant 3, mRNA. |
| 5860377 | ABCG1 | 9619 | Homo sapiens ATP-binding cassette, sub-family G |
| | | | (WHITE), member 1 (ABCG1), transcript variant 2, mRNA. |
| 2230730 | SRBD1 | 55133 | Homo sapiens S1 RNA binding domain 1 (SRBD1), mRNA. |
| 3130291 | C12orf57 | 113246 | Homo sapiens chromosome 12 open reading frame 57 |
| | | | (C12orf57), mRNA. |
| 5910039 | NAT6 | 24142 | Homo sapiens N-acetyltransferase 6 (GCN5-related) |
| | | | (NAT6), mRNA. |
| 6650333 | LOC653867 | 653867 | PREDICTED: Homo sapiens similar to Occludin |
| | | | (LOC653867), mRNA. |
| 5890075 | LIN7A | 8825 | Homo sapiens lin-7 homolog A (C. elegans) (LIN7A), |
| | | | mRNA. |
| 3360431 | ZAK | 51776 | Homo sapiens sterile alpha motif and leucine zipper |
| | | | containing kinase AZK (ZAK), transcript variant 1, mRNA. |
| 5390328 | KCNJ15 | 3772 | Homo sapiens potassium inwardly-rectifying channel, |
| | | | subfamily J, member 15 (KCNJ15), transcript variant 1, |
| | | | mRNA. |
| 1050215 | KCNJ15 | 3772 | Homo sapiens potassium inwardly-rectifying channel, |
| | | | subfamily J, member 15 (KCNJ15), transcript variant 1, |
| | | | mRNA. |
| 6020097 | SERPINA1 | 5265 | Homo sapiens serpin peptidase inhibitor, clade A (alpha-1 |
| | | | antiproteinase, antitrypsin), member 1 (SERPINA1), |
| | | | transcript variant 2, mRNA. |
| 620403 | LOC400759 | 400759 | Homo sapiens similar to Interferon-induced guanylate- |
| | | | binding protein 1 (GTP-binding protein 1) (Guanine |
| | | | nucleotide-binding protein 1) (HuGBP-1) (LOC400759) on |
| | | | chromosome 1. |
| 6280470 | LOC728519 | 728519 | PREDICTED: Homo sapiens similar to Baculoviral IAP |
| | | | repeat-containing protein 1 (Neuronal apoptosis inhibitory |
| | | | protein) (LOC728519), mRNA. |
| 6560079 | SUCNR1 | 56670 | Homo sapiens succinate receptor 1 (SUCNR1), mRNA. |
| 160070 | C16orf30 | 79652 | Homo sapiens chromosome 16 open reading frame 30 |
| | | | (C16orf30), mRNA. |
| 5570114 | GADD45G | 10912 | Homo sapiens growth arrest and DNA-damage-inducible, |
| | | | gamma (GADD45G), mRNA. |
| 7320424 | HNRPA1L-2 | 664709 | Homo sapiens heterogeneous nuclear ribonucleoprotein A1 |
| | | | pseudogene (HNRPA1L-2), non-coding RNA. |
| 3400612 | WDFY1 | 57590 | Homo sapiens WD repeat and FYVE domain containing 1 |
| | | | (WDFY1), mRNA. |
| 2570161 | TPD52 | 7163 | Homo sapiens tumor protein D52 (TPD52), transcript |
| | | | variant 3, mRNA. |
| 5490743 | KIF1B | 23095 | Homo sapiens kinesin family member 1B (KIF1B), |
| | | | transcript variant 2, mRNA. |
| 3370242 | DKFZp761P0423 | 157285 | PREDICTED: Homo sapiens hypothetical protein |
| | | | DKFZp761P0423 (DKFZp761P0423), mRNA. |
| 3390121 | CASP5 | 838 | Homo sapiens caspase 5, apoptosis-related cysteine |
| | | | peptidase (CASP5), mRNA. |
| 4760433 | C16orf7 | 9605 | Homo sapiens chromosome 16 open reading frame 7 |
| | | | (C16orf7), mRNA. |
| 6370435 | ETS1 | 2113 | Homo sapiens v-ets erythroblastosis virus E26 oncogene |
| | | | homolog 1 (avian) (ETS1), mRNA. |
| 4890722 | CD28 | 940 | Homo sapiens CD28 molecule (CD28), mRNA. |
| 2320689 | LOC653610 | 653610 | PREDICTED: Homo sapiens similar to Histone H2A.o |
| | | | (H2A/o) (H2A.2) (H2a-615) (LOC653610), mRNA. |
| 5130767 | MAPK14 | 1432 | Homo sapiens mitogen-activated protein kinase 14 |
| | | | (MAPK14), transcript variant 1, mRNA. |
| 1260270 | AIM2 | 9447 | Homo sapiens absent in melanoma 2 (AIM2), mRNA. |
| 1340500 | CLEC12A | 160364 | Homo sapiens C-type lectin domain family 12, member A |
| | | | (CLEC12A), transcript variant 1, mRNA. |
| 3780358 | CLEC12A | 160364 | Homo sapiens C-type lectin domain family 12, member A |
| | | | (CLEC12A), transcript variant 1, mRNA. |
| 2230044 | DOCK9 | 23348 | Homo sapiens dedicator of cytokinesis 9 (DOCK9), mRNA. |
| 3990433 | ADAP2 | 55803 | Homo sapiens ArfGAP with dual PH domains 2 (ADAP2), |
| | | | mRNA. |
| 3830471 | KIAA0748 | 9840 | PREDICTED: Homo sapiens KIAA0748 gene product, |
| | | | transcript variant 2 (KIAA0748), mRNA. |
| 5390427 | NAIP | 4671 | Homo sapiens NLR family, apoptosis inhibitory protein |
| | | | (NAIP), transcript variant 1, mRNA. |
| 5340240 | NAIP | 4671 | Homo sapiens NLR family, apoptosis inhibitory protein |
| | | | (NAIP), transcript variant 1, mRNA. |
| 7550358 | NELL2 | 4753 | Homo sapiens NEL-like 2 (chicken) (NELL2), mRNA. |
| 4860600 | MAPK14 | 1432 | Homo sapiens mitogen-activated protein kinase 14 |
| | | | (MAPK14), transcript variant 3, mRNA. |
| 1010360 | GCH1 | 2643 | Homo sapiens GTP cyclohydrolase 1 (GCH1), transcript |
| | | | variant 3, mRNA. |
| 1260021 | ZBP1 | 81030 | Homo sapiens Z-DNA binding protein 1 (ZBP1), mRNA. |
| 4010270 | LOC440731 | 440731 | PREDICTED: Homo sapiens hypothetical LOC440731, |
| | | | transcript variant 2 (LOC440731), mRNA. |
| 3850435 | AGPAT3 | 56894 | Homo sapiens 1-acylglycerol-3-phosphate O-acyltransferase |
| | | | 3 (AGPAT3), mRNA. |
| 60470 | STX11 | 8676 | Homo sapiens syntaxin 11 (STX11), mRNA. |
| 5910019 | C1QB | 713 | Homo sapiens complement component 1, q subcomponent, |
| | | | B chain (C1QB), mRNA. |
| 2710709 | FCGR1B | 2210 | Homo sapiens Fc fragment of IgG, high affinity Ib, receptor |
| | | | (CD64) (FCGR1B), transcript variant 1, mRNA. |
| 1010672 | NR3C2 | 4306 | Homo sapiens nuclear receptor subfamily 3, group C, |
| | | | member 2 (NR3C2), mRNA. |
| 4040037 | EBI2 | 1880 | Homo sapiens Epstein-Barr virus induced gene 2 |
| | | | (lymphocyte-specific G protein-coupled receptor) (EBI2), |
| | | | mRNA. |
| 4490520 | EBI2 | 1880 | Homo sapiens Epstein-Barr virus induced gene 2 |
| | | | (lymphocyte-specific G protein-coupled receptor) (EBI2), |
| | | | mRNA. |
| 2120746 | | | EST10086 human nasopharynx Homo sapiens cDNA, |
| | | | mRNA sequence |
| 830440 | TLR5 | 7100 | Homo sapiens toll-like receptor 5 (TLR5), mRNA. |
| 3840554 | SPOCK2 | 9806 | Homo sapiens sparc/osteonectin, cwcv and kazal-like |
| | | | domains proteoglycan (testican) 2 (SPOCK2), mRNA. |
| 1260482 | GZMK | 3003 | Homo sapiens granzyme K (granzyme 3; tryptase II) |
| | | | (GZMK), mRNA. |
| 3930368 | INCA | 440068 | Homo sapiens inhibitory caspase recruitment domain |
| | | | (CARD) protein (INCA), mRNA. |
| 5260392 | PRDM1 | 639 | Homo sapiens PR domain containing 1, with ZNF domain |
| | | | (PRDM1), transcript variant 1, mRNA. |
| 7050056 | SRPK1 | 6732 | Homo sapiens SFRS protein kinase 1 (SRPK1), mRNA. |
| 6480064 | POLR1E | 64425 | Homo sapiens polymerase (RNA) I polypeptide E, 53 kDa |
| | | | (POLR1E), mRNA. |
| 360132 | LHFPL2 | 10184 | Homo sapiens lipoma HMGIC fusion partner-like 2 |
| | | | (LHFPL2), mRNA. |
| 1050025 | CR1 | 1378 | Homo sapiens complement component (3b/4b) receptor 1 |
| | | | (Knops blood group) (CR1), transcript variant F, mRNA. |
| 4640184 | KIAA1026 | 23254 | Homo sapiens kazrin (KIAA1026), transcript variant B, |
| | | | mRNA. |
| 1990427 | KIAA1026 | 23254 | Homo sapiens kazrin (KIAA1026), transcript variant B, |
| | | | mRNA. |
| 3840215 | FAM102A | 399665 | Homo sapiens family with sequence similarity 102, member |
| | | | A (FAM102A), transcript variant 1, mRNA. |
| 1850546 | FAM102A | 399665 | Homo sapiens family with sequence similarity 102, member |
| | | | A (FAM102A), transcript variant 1, mRNA. |
| 6860450 | ZNF525 | 170958 | PREDICTED: Homo sapiens zinc finger protein 525 |
| | | | (ZNF525), mRNA. |
| 5130440 | BIN1 | 274 | Homo sapiens bridging integrator 1 (BIN1), transcript |
| | | | variant 4, mRNA. |
| 6450326 | CHMP5 | 51510 | Homo sapiens chromatin modifying protein 5 (CHMP5), |
| | | | mRNA. |
| 6420594 | TIFA | 92610 | Homo sapiens TRAF-interacting protein with forkhead- |
| | | | associated domain (TIFA), mRNA. |
| 5690228 | CDK5RAP2 | 55755 | Homo sapiens CDK5 regulatory subunit associated protein 2 |
| | | | (CDK5RAP2), transcript variant 1, mRNA. |
| 5270544 | GRAP | 10750 | Homo sapiens GRB2-related adaptor protein (GRAP), |
| | | | mRNA. |
| 6650482 | EPHA4 | 2043 | Homo sapiens EPH receptor A4 (EPHA4), mRNA. |
| 1070367 | C19orf59 | 199675 | Homo sapiens chromosome 19 open reading frame 59 |
| | | | (C19orf59), mRNA. |
| 4260017 | CDK5RAP2 | 55755 | Homo sapiens CDK5 regulatory subunit associated protein 2 |
| | | | (CDK5RAP2), transcript variant 2, mRNA. |
| 3890609 | PLSCR1 | 5359 | Homo sapiens phospholipid scramblase 1 (PLSCR1), |
| | | | mRNA. |
| 870202 | TNFSF10 | 8743 | Homo sapiens tumor necrosis factor (ligand) superfamily, |
| | | | member 10 (TNFSF10), mRNA. |
| 3780382 | DKFZp761E198 | 91056 | Homo sapiens DKFZp761E198 protein (DKFZp761E198), |
| | | | mRNA. |
| 4150692 | PARP14 | 54625 | Homo sapiens poly (ADP-ribose) polymerase family, |
| | | | member 14 (PARP14), mRNA. |
| 5700735 | PARP9 | 83666 | Homo sapiens poly (ADP-ribose) polymerase family, |
| | | | member 9 (PARP9), mRNA. |
| 3140707 | PARP9 | 83666 | Homo sapiens poly (ADP-ribose) polymerase family, |
| | | | member 9 (PARP9), mRNA. |
| 1940132 | CD151 | 977 | Homo sapiens CD151 molecule (Raph blood group) |
| | | | (CD151), transcript variant 4, mRNA. |
| 6650064 | SIGLEC5 | 8778 | Homo sapiens sialic acid binding Ig-like lectin 5 |
| | | | (SIGLEC5), mRNA. |
| 5290292 | DHRS9 | 10170 | Homo sapiens dehydrogenase/reductase (SDR family) |
| | | | member 9 (DHRS9), transcript variant 2, mRNA. |
| 2470601 | IL1RN | 3557 | Homo sapiens interleukin 1 receptor antagonist (IL1RN), |
| | | | transcript variant 1, mRNA. |
| 990768 | OAS3 | 4940 | Homo sapiens 2′-5′-oligoadenylate synthetase 3, 100 kDa |
| | | | (OAS3), mRNA. |
| 6900521 | PHTF1 | 10745 | Homo sapiens putative homeodomain transcription factor 1 |
| | | | (PHTF1), mRNA. |
| 5340767 | CEACAM1 | 634 | Homo sapiens carcinoembryonic antigen-related cell |
| | | | adhesion molecule 1 (biliary glycoprotein) (CEACAM1), |
| | | | transcript variant 1, mRNA. |
| 450189 | XAF1 | 54739 | Homo sapiens XIAP associated factor 1 (XAF1), transcript |
| | | | variant 2, mRNA. |
| 7610440 | XAF1 | 54739 | Homo sapiens XIAP associated factor 1 (XAF1), transcript |
| | | | variant 2, mRNA. |
| 4890327 | IFIT5 | 24138 | Homo sapiens interferon-induced protein with |
| | | | tetratricopeptide repeats 5 (IFIT5), mRNA. |
| 6380411 | IKZF3 | 22806 | Homo sapiens IKAROS family zinc finger 3 (Aiolos) |
| | | | (IKZF3), transcript variant 1, mRNA. |
| 3710152 | TRIM5 | 85363 | Homo sapiens tripartite motif-containing 5 (TRIM5), |
| | | | transcript variant gamma, mRNA. |
| 380050 | RHBDF2 | 79651 | Homo sapiens rhomboid 5 homolog 2 (Drosophila) |
| | | | (RHBDF2), transcript variant 1, mRNA. |
| 3060543 | MORC2 | 22880 | Homo sapiens MORC family CW-type zinc finger 2 |
| | | | (MORC2), mRNA. |
| 5560193 | BCAS4 | 55653 | Homo sapiens breast carcinoma amplified sequence 4 |
| | | | (BCAS4), transcript variant 1, mRNA. |
| 3310376 | TIMM10 | 26519 | Homo sapiens translocase of inner mitochondrial membrane |
| | | | 10 homolog (yeast) (TIMM10), nuclear gene encoding |
| | | | mitochondrial protein, mRNA. |
| 7330392 | TAP1 | 6890 | Homo sapiens transporter 1, ATP-binding cassette, sub- |
| | | | family B (MDR/TAP) (TAP1), mRNA. |
| 6400022 | CAPN12 | 147968 | Homo sapiens calpain 12 (CAPN12), mRNA. |
| 5700754 | APOL2 | 23780 | Homo sapiens apolipoprotein L, 2 (APOL2), transcript |
| | | | variant beta, mRNA. |
| 4150100 | PASK | 23178 | Homo sapiens PAS domain containing serine/threonine |
| | | | kinase (PASK), mRNA. |
| 2140382 | PASK | 23178 | Homo sapiens PAS domain containing serine/threonine |
| | | | kinase (PASK), mRNA. |
| 6510707 | FER1L3 | 26509 | Homo sapiens fer-1-like 3, myoferlin (C. elegans) |
| | | | (FER1L3), transcript variant 1, mRNA. |
| 3830497 | KLF12 | 11278 | Homo sapiens Kruppel-like factor 12 (KLF12), transcript |
| | | | variant 2, mRNA. |
| 870408 | IL15 | 3600 | Homo sapiens interleukin 15 (IL15), transcript variant 3, |
| | | | mRNA. |
| 770253 | KIAA1618 | 57714 | PREDICTED: Homo sapiens KIAA1618 (KIAA1618), |
| | | | mRNA. |
| 4290619 | CREB5 | 9586 | Homo sapiens cAMP responsive element binding protein 5 |
| | | | (CREB5), transcript variant 4, mRNA. |
| 3360343 | RSAD2 | 91543 | Homo sapiens radical S-adenosyl methionine domain |
| | | | containing 2 (RSAD2), mRNA. |
| 1010246 | IFI6 | 2537 | Homo sapiens interferon, alpha-inducible protein 6 (IFI6), |
| | | | transcript variant 2, mRNA. |
| 1090390 | OAS1 | 4938 | Homo sapiens 2′,5′-oligoadenylate synthetase 1, 40/46 kDa |
| | | | (OAS1), transcript variant 3, mRNA. |
| 7040035 | OAS1 | 4938 | Homo sapiens 2′,5′-oligoadenylate synthetase 1, 40/46 kDa |
| | | | (OAS1), transcript variant 3, mRNA. |
| 7150661 | HK2 | 3099 | Homo sapiens hexokinase 2 (HK2), mRNA. |
| 2140020 | LIMK2 | 3985 | Homo sapiens LIM domain kinase 2 (LIMK2), transcript |
| | | | variant 2b, mRNA. |
| 3390048 | LIMK2 | 3985 | Homo sapiens LIM domain kinase 2 (LIMK2), transcript |
| | | | variant 2b, mRNA. |
| 5910632 | SMARCD3 | 6604 | Homo sapiens SWI/SNF related, matrix associated, actin |
| | | | dependent regulator of chromatin, subfamily d, member 3 |
| | | | (SMARCD3), transcript variant 2, mRNA. |
| 7320139 | SOX8 | 30812 | Homo sapiens SRY (sex determining region Y)-box 8 |
| | | | (SOX8), mRNA. |
| 6860240 | | | Homo sapiens T cell receptor alpha locus, mRNA (cDNA |
| | | | clone MGC: 88342 IMAGE: 30352166), complete cds |
| 3170136 | SAMD9L | 219285 | Homo sapiens sterile alpha motif domain containing 9-like |
| | | | (SAMD9L), mRNA. |
| 10201 | URG4 | 55665 | Homo sapiens up-regulated gene 4 (URG4), nuclear gene |
| | | | encoding mitochondrial protein, transcript variant 1, mRNA. |
| 4760601 | LOC728014 | 728014 | PREDICTED: Homo sapiens similar to huntingtin |
| | | | interacting protein 1 related (LOC728014), mRNA. |
| 4900747 | C11orf75 | 56935 | Homo sapiens chromosome 11 open reading frame 75 |
| | | | (C11orf75), mRNA. |
| 1820114 | LILRB4 | 11006 | Homo sapiens leukocyte immunoglobulin-like receptor, |
| | | | subfamily B (with TM and ITIM domains), member 4 |
| | | | (LILRB4), transcript variant 2, mRNA. |
| 130519 | STAT2 | 6773 | Homo sapiens signal transducer and activator of |
| | | | transcription 2, 113 kDa (STAT2), mRNA. |
| 1240358 | RAB20 | 55647 | Homo sapiens RAB20, member RAS oncogene family |
| | | | (RAB20), mRNA. |
| 1990300 | SOCS1 | 8651 | Homo sapiens suppressor of cytokine signaling 1 (SOCS1), |
| | | | mRNA. |
| 630315 | DHRS9 | 10170 | Homo sapiens dehydrogenase/reductase (SDR family) |
| | | | member 9 (DHRS9), transcript variant 1, mRNA. |
| 830403 | PSME2 | 5721 | Homo sapiens proteasome (prosome, macropain) activator |
| | | | subunit 2 (PA28 beta) (PSME2), mRNA. |
| 4890255 | AGMAT | 79814 | Homo sapiens agmatine ureohydrolase (agmatinase) |
| | | | (AGMAT), mRNA. |
| 2100196 | ISG15 | 9636 | Homo sapiens ISG15 ubiquitin-like modifier (ISG15), |
| | | | mRNA. |
| 3170601 | CLEC12A | 160364 | Homo sapiens C-type lectin domain family 12, member A |
| | | | (CLEC12A), transcript variant 2, mRNA. |
| 3290292 | LAP3 | 51056 | Homo sapiens leucine aminopeptidase 3 (LAP3), mRNA. |
| 4900435 | TNFSF13B | 10673 | Homo sapiens tumor necrosis factor (ligand) superfamily, |
| | | | member 13b (TNFSF13B), mRNA. |
| 1240553 | SESN1 | 27244 | Homo sapiens sestrin 1 (SESN1), mRNA. |
| 4850592 | P2RY14 | 9934 | Homo sapiens purinergic receptor P2Y, G-protein coupled, |
| | | | 14 (P2RY14), transcript variant 2, mRNA. |
| 1340364 | P2RY14 | 9934 | Homo sapiens purinergic receptor P2Y, G-protein coupled, |
| | | | 14 (P2RY14), transcript variant 2, mRNA. |
| 6510170 | IFIT3 | 3437 | Homo sapiens interferon-induced protein with |
| | | | tetratricopeptide repeats 3 (IFIT3), mRNA. |
| 1500280 | IFIT3 | 3437 | Homo sapiens interferon-induced protein with |
| | | | tetratricopeptide repeats 3 (IFIT3), mRNA. |
| 5270709 | ZNF429 | 353088 | Homo sapiens zinc finger protein 429 (ZNF429), mRNA. |
| 1440360 | GPR141 | 353345 | Homo sapiens G protein-coupled receptor 141 (GPR141), |
| | | | mRNA. |
| 5860521 | SLC30A1 | 7779 | Homo sapiens solute carrier family 30 (zinc transporter), |
| | | | member 1 (SLC30A1), mRNA. |
| 3890523 | IL7R | 3575 | PREDICTED: Homo sapiens interleukin 7 receptor (IL7R), |
| | | | mRNA. |
| 1980356 | | | af01b06.s1 Human bone marrow stromal cells Homo sapiens |
| | | | cDNA clone IMAGE: 1027283 3, mRNA sequence |
| 4120053 | PFKFB3 | 5209 | Homo sapiens 6-phosphofructo-2-kinase/fructose-2,6- |
| | | | biphosphatase 3 (PFKFB3), mRNA. |
| 4150270 | ANKRD22 | 118932 | Homo sapiens ankyrin repeat domain 22 (ANKRD22), |
| | | | mRNA. |
| 50706 | CD40LG | 959 | Homo sapiens CD40 ligand (CD40LG), mRNA. |
| 3990673 | AFF1 | 4299 | Homo sapiens AF4/FMR2 family, member 1 (AFF1), |
| | | | mRNA. |
| 7610113 | SLC26A8 | 116369 | Homo sapiens solute carrier family 26, member 8 |
| | | | (SLC26A8), transcript variant 2, mRNA. |
| 5360626 | SLC26A8 | 116369 | Homo sapiens solute carrier family 26, member 8 |
| | | | (SLC26A8), transcript variant 2, mRNA. |
| 2070170 | UBE2L6 | 9246 | Homo sapiens ubiquitin-conjugating enzyme E2L 6 |
| | | | (UBE2L6), transcript variant 1, mRNA. |
| 7000368 | UBE2L6 | 9246 | Homo sapiens ubiquitin-conjugating enzyme E2L 6 |
| | | | (UBE2L6), transcript variant 1, mRNA. |
| 770088 | DTX3L | 151636 | Homo sapiens deltex 3-like (Drosophila) (DTX3L), mRNA. |
| 5670465 | ADM | 133 | Homo sapiens adrenomedullin (ADM), mRNA. |
| 1820592 | HIST2H2AA3 | 8337 | Homo sapiens histone cluster 2, H2aa3 (HIST2H2AA3), |
| | | | mRNA. |
| 610451 | HIST2H2AA3 | 8337 | Homo sapiens histone cluster 2, H2aa3 (HIST2H2AA3), |
| | | | mRNA. |
| 5220142 | LIMK2 | 3985 | Homo sapiens LIM domain kinase 2 (LIMK2), transcript |
| | | | variant 2a, mRNA. |
| 670528 | GYG1 | 2992 | Homo sapiens glycogenin 1 (GYG1), mRNA. |
| 110397 | LOC652755 | 652755 | PREDICTED: Homo sapiens similar to Baculoviral IAP |
| | | | repeat-containing protein 1 (Neuronal apoptosis inhibitory |
| | | | protein) (LOC652755), mRNA. |
| 450348 | GNG10 | 2790 | Homo sapiens guanine nucleotide binding protein (G |
| | | | protein), gamma 10 (GNG10), mRNA. |
| 3420523 | RHBDF2 | 79651 | Homo sapiens rhomboid 5 homolog 2 (Drosophila) |
| | | | (RHBDF2), transcript variant 2, mRNA. |
| 650750 | RHBDF2 | 79651 | Homo sapiens rhomboid 5 homolog 2 (Drosophila) |
| | | | (RHBDF2), transcript variant 2, mRNA. |
| 4570075 | KIAA1641 | 57730 | PREDICTED: Homo sapiens KIAA1641, transcript variant |
| | | | 7 (KIAA1641), mRNA. |
| 1470132 | B3GNT8 | 374907 | Homo sapiens UDP-GlcNAc: betaGal beta-1,3-N- |
| | | | acetylglucosaminyltransferase 8 (B3GNT8), mRNA. |
| 2810246 | LBH | 81606 | Homo sapiens limb bud and heart development homolog |
| | | | (mouse) (LBH), mRNA. |
| 5050274 | DHRS12 | 79758 | Homo sapiens dehydrogenase/reductase (SDR family) |
| | | | member 12 (DHRS12), transcript variant 2, mRNA. |
| 3710086 | DHRS12 | 79758 | Homo sapiens dehydrogenase/reductase (SDR family) |
| | | | member 12 (DHRS12), transcript variant 2, mRNA. |
| 4210411 | NDRG2 | 57447 | Homo sapiens NDRG family member 2 (NDRG2), transcript |
| | | | variant 6, mRNA. |
| 6980133 | | | HUMGS0004661 Human adult (K. Okubo) Homo sapiens |
| | | | cDNA 3, mRNA sequence |
| 6590646 | FAM26F | 441168 | Homo sapiens family with sequence similarity 26, member F |
| | | | (FAM26F), mRNA. |
| 3780193 | FCRLA | 84824 | Homo sapiens Fc receptor-like A (FCRLA), mRNA. |
| 1070477 | ALDH1A1 | 216 | Homo sapiens aldehyde dehydrogenase 1 family, member |
| | | | A1 (ALDH1A1), mRNA. |
| 830615 | ZDHHC19 | 131540 | Homo sapiens zinc finger, DHHC-type containing 19 |
| | | | (ZDHHC19), mRNA. |
| 3940484 | MEF2D | 4209 | Homo sapiens myocyte enhancer factor 2D (MEF2D), |
| | | | mRNA. |
| 1410201 | CD59 | 966 | Homo sapiens CD59 molecule, complement regulatory |
| | | | protein (CD59), transcript variant 2, mRNA. |
| 1050360 | HLA-DPB1 | 3115 | Homo sapiens major histocompatibility complex, class II, |
| | | | DP beta 1 (HLA-DPB1), mRNA. |
| 2230129 | CLUAP1 | 23059 | Homo sapiens clusterin associated protein 1 (CLUAP1), |
| | | | transcript variant 2, mRNA. |
| 4900239 | CD274 | 29126 | Homo sapiens CD274 molecule (CD274), mRNA. |
| 940274 | GK | 2710 | Homo sapiens glycerol kinase (GK), transcript variant 2, |
| | | | mRNA. |
| 3940189 | JAK2 | 3717 | Homo sapiens Janus kinase 2 (a protein tyrosine kinase) |
| | | | (JAK2), mRNA. |
| 2750280 | CDK5R1 | 8851 | Homo sapiens cyclin-dependent kinase 5, regulatory subunit |
| | | | 1 (p35) (CDK5R1), mRNA. |
| 6650242 | IFITM3 | 10410 | Homo sapiens interferon induced transmembrane protein 3 |
| | | | (1-8 U) (IFITM3), mRNA. |
| 2070044 | CLYBL | 171425 | Homo sapiens citrate lyase beta like (CLYBL), mRNA. |
| 1820750 | STAT1 | 6772 | Homo sapiens signal transducer and activator of |
| | | | transcription 1, 91 kDa (STAT1), transcript variant alpha, |
| | | | mRNA. |
| 4810187 | STAT1 | 6772 | Homo sapiens signal transducer and activator of |
| | | | transcription 1, 91 kDa (STAT1), transcript variant alpha, |
| | | | mRNA. |
| 3440433 | | | BX092531 NCI_CGAP_Kid5 Homo sapiens cDNA clone |
| | | | IMAGp998I114659; IMAGE: 1900882, mRNA sequence |
| 4150189 | CTSL1 | 1514 | Homo sapiens cathepsin L1 (CTSL1), transcript variant 1, |
| | | | mRNA. |
| 1260066 | | | Homo sapiens cDNA: FLJ21199 fis, clone COL00235 |
| 6250615 | PGLYRP1 | 8993 | Homo sapiens peptidoglycan recognition protein 1 |
| | | | (PGLYRP1), mRNA. |
| 3870338 | IFI44L | 10964 | Homo sapiens interferon-induced protein 44-like (IFI44L), |
| | | | mRNA. |
| 3930601 | PARP10 | 84875 | PREDICTED: Homo sapiens poly (ADP-ribose) polymerase |
| | | | family, member 10 (PARP10), mRNA. |
| 160672 | TCF7 | 6932 | Homo sapiens transcription factor 7 (T-cell specific, HMG- |
| | | | box) (TCF7), transcript variant 2, mRNA. |
| 610400 | C11orf1 | 64776 | Homo sapiens chromosome 11 open reading frame 1 |
| | | | (C11orf1), mRNA. |
| 1240450 | CD27 | 939 | Homo sapiens CD27 molecule (CD27), mRNA. |
| 6100022 | HIST2H2AC | 8338 | Homo sapiens histone cluster 2, H2ac (HIST2H2AC), |
| | | | mRNA. |
| 5270520 | FAIM3 | 9214 | Homo sapiens Fas apoptotic inhibitory molecule 3 (FAIM3), |
| | | | mRNA. |
| 7510537 | SCO2 | 9997 | Homo sapiens SCO cytochrome oxidase deficient homolog |
| | | | 2 (yeast) (SCO2), nuclear gene encoding mitochondrial |
| | | | protein, mRNA. |
| 6620487 | CXCL6 | 6372 | Homo sapiens chemokine (C-X-C motif) ligand 6 |
| | | | (granulocyte chemotactic protein 2) (CXCL6), mRNA. |
| 3930128 | | | BX110640 Soares_testis_NHT Homo sapiens cDNA clone |
| | | | IMAGp998B094156, mRNA sequence |
| 6520180 | IL18RAP | 8807 | Homo sapiens interleukin 18 receptor accessory protein |
| | | | (IL18RAP), mRNA. |
| 2000110 | BRSK1 | 84446 | Homo sapiens BR serine/threonine kinase 1 (BRSK1), |
| | | | mRNA. |
| 3140487 | BMX | 660 | Homo sapiens BMX non-receptor tyrosine kinase (BMX), |
| | | | mRNA. |
| 4760703 | DDX58 | 23586 | Homo sapiens DEAD (Asp-Glu-Ala-Asp) box polypeptide |
| | | | 58 (DDX58), mRNA. |
| 3990224 | TNFRSF25 | 8718 | Homo sapiens tumor necrosis factor receptor superfamily, |
| | | | member 25 (TNFRSF25), transcript variant 10, mRNA. |
| 5720482 | HERC5 | 51191 | Homo sapiens hect domain and RLD 5 (HERC5), mRNA. |
| 2570079 | STAT1 | 6772 | Homo sapiens signal transducer and activator of |
| | | | transcription 1, 91 kDa (STAT1), transcript variant beta, |
| | | | mRNA. |
| 520086 | FCGR1A | 2209 | Homo sapiens Fc fragment of IgG, high affinity Ia, receptor |
| | | | (CD64) (FCGR1A), mRNA. |
| 2570300 | IFI44 | 10561 | Homo sapiens interferon-induced protein 44 (IFI44), |
| | | | mRNA. |
| 3890592 | | | RST5526 Athersys RAGE Library Homo sapiens cDNA, |
| | | | mRNA sequence |
| 6280543 | OASL | 8638 | Homo sapiens 2′-5′-oligoadenylate synthetase-like (OASL), |
| | | | transcript variant 1, mRNA. |
| 630619 | HPSE | 10855 | Homo sapiens heparanase (HPSE), mRNA. |
| 6620689 | MTHFD2 | 10797 | Homo sapiens methylenetetrahydrofolate dehydrogenase |
| | | | (NADP+ dependent) 2, methenyltetrahydrofolate |
| | | | cyclohydrolase (MTHFD2), nuclear gene encoding |
| | | | mitochondrial protein, transcript variant 2, mRNA. |
| 6370470 | APOL1 | 8542 | Homo sapiens apolipoprotein L, 1 (APOL1), transcript |
| | | | variant 2, mRNA. |
| 6290519 | STOM | 2040 | Homo sapiens stomatin (STOM), transcript variant 1, |
| | | | mRNA. |
| 5560471 | FCAR | 2204 | Homo sapiens Fc fragment of IgA, receptor for (FCAR), |
| | | | transcript variant 10, mRNA. |
| 6960168 | ATP8B2 | 57198 | Homo sapiens ATPase, class I, type 8B, member 2 |
| | | | (ATP8B2), transcript variant 1, mRNA. |
| 2030170 | COP1 | 114769 | Homo sapiens caspase-1 dominant-negative inhibitor |
| | | | pseudo-ICE (COP1), transcript variant 2, mRNA. |
| 6400681 | SP140 | 11262 | Homo sapiens SP140 nuclear body protein (SP140), |
| | | | transcript variant 2, mRNA. |
| 4890292 | TSHZ2 | 128553 | Homo sapiens teashirt zinc finger homeobox 2 (TSHZ2), |
| | | | mRNA. |
| 2450707 | SIGLEC16 | 400709 | Homo sapiens sialic acid binding Ig-like lectin 16 |
| | | | (gene/pseudogene) (SIGLEC16), non-coding RNA. |
| 5570039 | LOC728744 | 728744 | PREDICTED: Homo sapiens hypothetical LOC728744 |
| | | | (LOC728744), mRNA. |
| 3710068 | WARS | 7453 | Homo sapiens tryptophanyl-tRNA synthetase (WARS), |
| | | | transcript variant 2, mRNA. |
| 3180528 | MMP9 | 4318 | Homo sapiens matrix metallopeptidase 9 (gelatinase B, |
| | | | 92 kDa gelatinase, 92 kDa type IV collagenase) (MMP9), |
| | | | mRNA. |
| 2650156 | TAP2 | 6891 | Homo sapiens transporter 2, ATP-binding cassette, sub- |
| | | | family B (MDR/TAP) (TAP2), transcript variant 2, mRNA. |
| 4280482 | DRAM | 55332 | Homo sapiens damage-regulated autophagy modulator |
| | | | (DRAM), mRNA. |
| 6200161 | CECR6 | 27439 | Homo sapiens cat eye syndrome chromosome region, |
| | | | candidate 6 (CECR6), mRNA. |
| 3840288 | LRFN3 | 79414 | Homo sapiens leucine rich repeat and fibronectin type III |
| | | | domain containing 3 (LRFN3), mRNA. |
| 4860753 | ASGR2 | 433 | Homo sapiens asialoglycoprotein receptor 2 (ASGR2), |
| | | | transcript variant 3, mRNA. |
| 3800576 | ASGR2 | 433 | Homo sapiens asialoglycoprotein receptor 2 (ASGR2), |
| | | | transcript variant 3, mRNA. |
| 2600747 | IFIT2 | 3433 | Homo sapiens interferon-induced protein with |
| | | | tetratricopeptide repeats 2 (IFIT2), mRNA. |
| 1980524 | GBP4 | 115361 | Homo sapiens guanylate binding protein 4 (GBP4), mRNA. |
| 6480403 | TXNDC3 | 51314 | Homo sapiens thioredoxin domain containing 3 |
| | | | (spermatozoa) (TXNDC3), mRNA. |
| 4570441 | IFIH1 | 64135 | Homo sapiens interferon induced with helicase C domain 1 |
| | | | (IFIH1), mRNA. |
| 5810689 | PRRG4 | 79056 | Homo sapiens proline rich Gla (G-carboxyglutamic acid) 4 |
| | | | (transmembrane) (PRRG4), mRNA. |
| 6980546 | CACNA1I | 8911 | Homo sapiens calcium channel, voltage-dependent, T type, |
| | | | alpha 1I subunit (CACNA1I), transcript variant 2, mRNA. |
| 5890470 | CCR6 | 1235 | Homo sapiens chemokine (C-C motif) receptor 6 (CCR6), |
| | | | transcript variant 2, mRNA. |
| 4920110 | GADD45B | 4616 | Homo sapiens growth arrest and DNA-damage-inducible, |
| | | | beta (GADD45B), mRNA. |
| 5670100 | TCN2 | 6948 | Homo sapiens transcobalamin II; macrocytic anemia |
| | | | (TCN2), mRNA. |
| 6180088 | ATP6V0E2 | 155066 | Homo sapiens ATPase, H+ transporting V0 subunit e2 |
| | | | (ATP6V0E2), transcript variant 1, mRNA. |
| 270240 | SLC26A8 | 116369 | Homo sapiens solute carrier family 26, member 8 |
| | | | (SLC26A8), transcript variant 1, mRNA. |
| 1260445 | SLC26A8 | 116369 | Homo sapiens solute carrier family 26, member 8 |
| | | | (SLC26A8), transcript variant 1, mRNA. |
| 4290037 | NOV | 4856 | Homo sapiens nephroblastoma overexpressed gene (NOV), |
| | | | mRNA. |
| 7610053 | DDX60 | 55601 | Homo sapiens DEAD (Asp-Glu-Ala-Asp) box polypeptide |
| | | | 60 (DDX60), mRNA. |
| 520441 | LOC642606 | 642606 | PREDICTED: Homo sapiens hypothetical protein |
| | | | LOC642606 (LOC642606), mRNA. |
| 5960747 | TRIM22 | 10346 | Homo sapiens tripartite motif-containing 22 (TRIM22), |
| | | | mRNA. |
| 3460014 | C4orf18 | 51313 | Homo sapiens chromosome 4 open reading frame 18 |
| | | | (C4orf18), transcript variant 2, mRNA. |
| 4860224 | WARS | 7453 | Homo sapiens tryptophanyl-tRNA synthetase (WARS), |
| | | | transcript variant 1, mRNA. |
| 1940162 | GBP2 | 2634 | Homo sapiens guanylate binding protein 2, interferon- |
| | | | inducible (GBP2), mRNA. |
| 3290072 | | | BX116726 NCI_CGAP_Pr28 Homo sapiens cDNA clone |
| | | | IMAGp998J065569, mRNA sequence |
| 1770243 | IGF2BP3 | 10643 | Homo sapiens insulin-like growth factor 2 mRNA binding |
| | | | protein 3 (IGF2BP3), mRNA. |
| 7650097 | OASL | 8638 | Homo sapiens 2′-5′-oligoadenylate synthetase-like (OASL), |
| | | | transcript variant 2, mRNA. |
| 4220187 | DYSF | 8291 | Homo sapiens dysferlin, limb girdle muscular dystrophy 2B |
| | | | (autosomal recessive) (DYSF), mRNA. |
| 2030360 | FAM159A | 348378 | Homo sapiens family with sequence similarity 159, member |
| | | | A (FAM159A), mRNA. |
| 540161 | LOC552891 | 552891 | Homo sapiens hypothetical protein LOC552891 |
| | | | (LOC552891), mRNA. |
| 5550397 | APOL6 | 80830 | Homo sapiens apolipoprotein L, 6 (APOL6), mRNA. |
| 520408 | IFIT3 | 3437 | Homo sapiens interferon-induced protein with |
| | | | tetratricopeptide repeats 3 (IFIT3), mRNA. |
| 4120367 | LPCAT2 | 54947 | Homo sapiens lysophosphatidylcholine acyltransferase 2 |
| | | | (LPCAT2), mRNA. |
| 1580411 | CD3D | 915 | Homo sapiens CD3d molecule, delta (CD3-TCR complex) |
| | | | (CD3D), transcript variant 2, mRNA. |
| 7160592 | ACOT9 | 23597 | Homo sapiens acyl-CoA thioesterase 9 (ACOT9), transcript |
| | | | variant 2, mRNA. |
| 6270553 | CXCL10 | 3627 | Homo sapiens chemokine (C-X-C motif) ligand 10 |
| | | | (CXCL10), mRNA. |
| 5890095 | LILRA5 | 353514 | Homo sapiens leukocyte immunoglobulin-like receptor, |
| | | | subfamily A (with TM domain), member 5 (LILRA5), |
| | | | transcript variant 1, mRNA. |
| 7650128 | ECGF1 | 1890 | Homo sapiens endothelial cell growth factor 1 (platelet- |
| | | | derived) (ECGF1), mRNA. |
| 5310451 | BCL6 | 604 | Homo sapiens B-cell CLL/lymphoma 6 (zinc finger protein |
| | | | 51) (BCL6), transcript variant 2, mRNA. |
| 5420349 | LOC648099 | 648099 | PREDICTED: Homo sapiens similar to positive cofactor 2, |
| | | | glutamine/Q-rich-associated protein isoform b |
| | | | (LOC648099), mRNA. |
| 4150369 | GK | 2710 | Homo sapiens glycerol kinase (GK), transcript variant 1, |
| | | | mRNA. |
| 4250136 | LTB4R | 1241 | Homo sapiens leukotriene B4 receptor (LTB4R), mRNA. |
| 6250187 | MGC22014 | 200424 | PREDICTED: Homo sapiens hypothetical protein |
| | | | MGC22014 (MGC22014), mRNA. |
| 1780224 | MGC42367 | 343990 | Homo sapiens similar to 2010300C02Rik protein |
| | | | (MGC42367), mRNA. |
| 6350634 | DYNLT1 | 6993 | Homo sapiens dynein, light chain, Tctex-type 1 (DYNLT1), |
| | | | mRNA. |
| 2600369 | KRT72 | 140807 | Homo sapiens keratin 72 (KRT72), mRNA. |
| 4670458 | Sep-04 | 5414 | Homo sapiens septin 4 (SEPT4), transcript variant 2, |
| | | | mRNA. |
| 240594 | ANXA3 | 306 | Homo sapiens annexin A3 (ANXA3), mRNA. |
| 4920228 | FFAR2 | 2867 | Homo sapiens free fatty acid receptor 2 (FFAR2), mRNA. |
| 10136 | PML | 5371 | PREDICTED: Homo sapiens promyelocytic leukemia, |
| | | | transcript variant 12 (PML), mRNA. |
| 1450427 | RTP4 | 64108 | Homo sapiens receptor (chemosensory) transporter protein 4 |
| | | | (RTP4), mRNA. |
| 3610706 | BMX | 660 | Homo sapiens BMX non-receptor tyrosine kinase (BMX), |
| | | | mRNA. |
| 3610343 | NBN | 4683 | Homo sapiens nibrin (NBN), mRNA. |
| 6400603 | PVRIG | 79037 | Homo sapiens poliovirus receptor related immunoglobulin |
| | | | domain containing (PVRIG), mRNA. |
| 6940605 | | | Homo sapiens cDNA clone IMAGE: 5277162 |
| 7560593 | OSM | 5008 | Homo sapiens oncostatin M (OSM), mRNA. |
| 3130139 | C9orf66 | 157983 | Homo sapiens chromosome 9 open reading frame 66 |
| | | | (C9orf66), mRNA. |
| 3780370 | C9orf72 | 203228 | Homo sapiens chromosome 9 open reading frame 72 |
| | | | (C9orf72), transcript variant 2, mRNA. |
| 2230097 | TNFRSF25 | 8718 | Homo sapiens tumor necrosis factor receptor superfamily, |
| | | | member 25 (TNFRSF25), transcript variant 12, mRNA. |
| 6840767 | FRMD3 | 257019 | Homo sapiens FERM domain containing 3 (FRMD3), |
| | | | mRNA. |
| 6100379 | LOC648984 | 648984 | PREDICTED: Homo sapiens similar to Baculoviral IAP |
| | | | repeat-containing protein 1 (Neuronal apoptosis inhibitory |
| | | | protein) (LOC648984), mRNA. |
| 6550600 | MYC | 4609 | Homo sapiens v-myc myelocytomatosis viral oncogene |
| | | | homolog (avian) (MYC), mRNA. |
| 3780047 | GBP6 | 163351 | Homo sapiens guanylate binding protein family, member 6 |
| | | | (GBP6), mRNA. |
| 7380400 | GBP6 | 163351 | Homo sapiens guanylate binding protein family, member 6 |
| | | | (GBP6), mRNA. |
| 5910064 | TRIM5 | 85363 | Homo sapiens tripartite motif-containing 5 (TRIM5), |
| | | | transcript variant delta, mRNA. |
| 4230102 | SOCS3 | 9021 | Homo sapiens suppressor of cytokine signaling 3 (SOCS3), |
| | | | mRNA. |
| 4290491 | PSME1 | 5720 | Homo sapiens proteasome (prosome, macropain) activator |
| | | | subunit 1 (PA28 alpha) (PSME1), transcript variant 2, |
| | | | mRNA. |
| 3610327 | OAS2 | 4939 | Homo sapiens 2′-5′-oligoadenylate synthetase 2, 69/71 kDa |
| | | | (OAS2), transcript variant 1, mRNA. |
| 1430360 | ABHD14A | 25864 | Homo sapiens abhydrolase domain containing 14A |
| | | | (ABHD14A), mRNA. |
| 7400102 | ASPHD2 | 57168 | Homo sapiens aspartate beta-hydroxylase domain containing |
| | | | 2 (ASPHD2), mRNA. |
| 7160468 | DHRS9 | 10170 | Homo sapiens dehydrogenase/reductase (SDR family) |
| | | | member 9 (DHRS9), transcript variant 1, mRNA. |
| 4490079 | DHRS9 | 10170 | Homo sapiens dehydrogenase/reductase (SDR family) |
| | | | member 9 (DHRS9), transcript variant 1, mRNA. |
| 2140242 | TNFAIP6 | 7130 | Homo sapiens tumor necrosis factor, alpha-induced protein 6 |
| | | | (TNFAIP6), mRNA. |

TABLE 7
List of the 565 genes

| ProbeID | Symbol | Entrez_Gene_ID | Definition |
|---|---|---|---|
| 2710020 | | | BX093329 Soares_parathyroid_tumor_NbHPA Homo |
| | | | sapiens cDNA clone IMAGp998A124183; |
| | | | IMAGE: 1648403, mRNA sequence |
| 6020544 | CLEC12A | 160364 | Homo sapiens C-type lectin domain family 12, member A |
| | | | (CLEC12A), transcript variant 3, mRNA. |
| 2190349 | KCNJ15 | 3772 | Homo sapiens potassium inwardly-rectifying channel, |
| | | | subfamily J, member 15 (KCNJ15), transcript variant 3, |
| | | | mRNA. |
| 5570139 | QPCT | 25797 | Homo sapiens glutaminyl-peptide cyclotransferase |
| | | | (QPCT), mRNA. |
| 5700725 | EPSTI1 | 94240 | Homo sapiens epithelial stromal interaction 1 |
| | | | (breast) (EPSTI1), transcript variant 2, mRNA. |
| 670594 | C2 | 717 | Homo sapiens complement component 2 (C2), mRNA. |
| 5220538 | WDFY3 | 23001 | Homo sapiens WD repeat and FYVE domain containing 3 |
| | | | (WDFY3), transcript variant 2, mRNA. |
| 2070646 | GPR84 | 53831 | Homo sapiens G protein-coupled receptor 84 (GPR84), |
| | | | mRNA. |
| 520162 | HPSE | 10855 | Homo sapiens heparanase (HPSE), mRNA. |
| 3850440 | FCER1G | 2207 | Homo sapiens Fc fragment of IgE, high affinity I, receptor |
| | | | for; gamma polypeptide (FCER1G), mRNA. |
| 130181 | ANKRD22 | 118932 | Homo sapiens ankyrin repeat domain 22 (ANKRD22), |
| | | | mRNA. |
| 620136 | LILRA5 | 353514 | Homo sapiens leukocyte immunoglobulin-like receptor, |
| | | | subfamily A (with TM domain), member 5 (LILRA5), |
| | | | transcript variant 3, mRNA. |
| 5860075 | CAMP | 820 | Homo sapiens cathelicidin antimicrobial peptide (CAMP), |
| | | | mRNA. |
| 2850241 | CAPNS2 | 84290 | Homo sapiens calpain, small subunit 2 (CAPNS2), |
| | | | mRNA. |
| 4290368 | PSTPIP2 | 9050 | Homo sapiens proline-serine-threonine phosphatase |
| | | | interacting protein 2 (PSTPIP2), mRNA. |
| 3930411 | CAMTA1 | 23261 | Homo sapiens calmodulin binding transcription activator 1 |
| | | | (CAMTA1), mRNA. |
| 2360348 | CMPK2 | 129607 | Homo sapiens cytidine monophosphate (UMP-CMP) |
| | | | kinase 2, mitochondrial (CMPK2), nuclear gene encoding |
| | | | mitochondrial protein, mRNA. |
| 3140402 | VRK2 | 7444 | Homo sapiens vaccinia related kinase 2 (VRK2), mRNA. |
| 70167 | LY96 | 23643 | Homo sapiens lymphocyte antigen 96 (LY96), mRNA. |
| 2630195 | VAMP5 | 10791 | Homo sapiens vesicle-associated membrane protein 5 |
| | | | (myobrevin) (VAMP5), mRNA. |
| 2370228 | CCDC65 | 85478 | Homo sapiens coiled-coil domain containing 65 |
| | | | (CCDC65), mRNA. |
| 4290731 | KREMEN1 | 83999 | Homo sapiens kringle containing transmembrane protein 1 |
| | | | (KREMEN1), transcript variant 2, mRNA. |
| 5700451 | FKBP9L | 360132 | Homo sapiens FK506 binding protein 9-like (FKBP9L), |
| | | | mRNA. |
| 4540333 | PIWIL4 | 143689 | Homo sapiens piwi-like 4 (Drosophila) (PIWIL4), mRNA. |
| 6620209 | FCGR1B | 2210 | Homo sapiens Fc fragment of IgG, high affinity Ib, |
| | | | receptor (CD64) (FCGR1B), transcript variant 2, mRNA. |
| 130609 | FCGBP | 8857 | Homo sapiens Fc fragment of IgG binding protein |
| | | | (FCGBP), mRNA. |
| 20048 | HIST2H2BF | 440689 | Homo sapiens histone cluster 2, H2bf (HIST2H2BF), |
| | | | mRNA. |
| 3990010 | | | AGENCOURT_7914287 NIH_MGC_71 Homo sapiens |
| | | | cDNA clone IMAGE: 6156595 5, mRNA sequence |
| 1510364 | GBP5 | 115362 | Homo sapiens guanylate binding protein 5 (GBP5), |
| | | | mRNA. |
| 670202 | SESTD1 | 91404 | Homo sapiens SEC14 and spectrin domains 1 (SESTD1), |
| | | | mRNA. |
| 6370768 | ETV7 | 51513 | Homo sapiens ets variant 7 (ETV7), mRNA. |
| 3440095 | PSG3 | 5671 | Homo sapiens pregnancy specific beta-1-glycoprotein 3 |
| | | | (PSG3), mRNA. |
| 5810309 | SIRPB2 | 284759 | PREDICTED: Homo sapiens signal-regulatory protein |
| | | | beta 2, transcript variant 4 (SIRPB2), mRNA. |
| 360014 | CDH23 | 64072 | Homo sapiens cadherin-like 23 (CDH23), transcript |
| | | | variant 2, mRNA. |
| 6400148 | SELM | 140606 | Homo sapiens selenoprotein M (SELM), mRNA. |
| 4280632 | GAS6 | 2621 | Homo sapiens growth arrest-specific 6 (GAS6), mRNA. |
| 70730 | GAS6 | 2621 | Homo sapiens growth arrest-specific 6 (GAS6), mRNA. |
| 4850356 | LOC642120 | 642120 | PREDICTED: Homo sapiens similar to Maltase- |
| | | | glucoamylase, intestinal (LOC642120), mRNA. |
| 1030100 | | | cl02h05.z1 Hembase; Erythroid Precursor Cells (LCB: cl |
| | | | library) Homo sapiens cDNA clone cl02h05 5, mRNA |
| | | | sequence |
| 1470091 | IL15 | 3600 | Homo sapiens interleukin 15 (IL15), transcript variant 1, |
| | | | mRNA. |
| 2490315 | | | Homo sapiens cDNA: FLJ23098 fis, clone LNG07440 |
| 110639 | MAPK14 | 1432 | Homo sapiens mitogen-activated protein kinase 14 |
| | | | (MAPK14), transcript variant 2, mRNA. |
| 5050041 | GPR109B | 8843 | Homo sapiens G protein-coupled receptor 109B |
| | | | (GPR109B), mRNA. |
| 4860255 | ASPRV1 | 151516 | Homo sapiens aspartic peptidase, retroviral-like 1 |
| | | | (ASPRV1), mRNA. |
| 6900475 | ABHD2 | 11057 | Homo sapiens abhydrolase domain containing 2 |
| | | | (ABHD2), transcript variant 2, mRNA. |
| 7330477 | SLITRK4 | 139065 | Homo sapiens SLIT and NTRK-like family, member 4 |
| | | | (SLITRK4), mRNA. |
| 1850156 | CLDN23 | 137075 | Homo sapiens claudin 23 (CLDN23), mRNA. |
| 6560156 | DUSP3 | 1845 | Homo sapiens dual specificity phosphatase 3 (vaccinia |
| | | | virus phosphatase VH1-related) (DUSP3), mRNA. |
| 380102 | TMEM51 | 55092 | Homo sapiens transmembrane protein 51 (TMEM51), |
| | | | mRNA. |
| 6550367 | PAQR6 | 79957 | Homo sapiens progestin and adipoQ receptor family |
| | | | member VI (PAQR6), transcript variant 1, mRNA. |
| 3520327 | LOC642161 | 642161 | PREDICTED: Homo sapiens similar to T-cell receptor |
| | | | beta chain V region CTL-L17 precursor (LOC642161), |
| | | | mRNA. |
| 6040577 | SLAMF8 | 56833 | Homo sapiens SLAM family member 8 (SLAMF8), |
| | | | mRNA. |
| 2760500 | CD38 | 952 | Homo sapiens CD38 molecule (CD38), mRNA. |
| 6560075 | TRIM21 | 6737 | Homo sapiens tripartite motif-containing 21 (TRIM21), |
| | | | mRNA. |
| 7570324 | ID3 | 3399 | Homo sapiens inhibitor of DNA binding 3, dominant |
| | | | negative helix-loop-helix protein (ID3), mRNA. |
| 3940356 | CR1 | 1378 | Homo sapiens complement component (3b/4b) receptor 1 |
| | | | (Knops blood group) (CR1), transcript variant S, mRNA. |
| 4200669 | CR1 | 1378 | Homo sapiens complement component (3b/4b) receptor 1 |
| | | | (Knops blood group) (CR1), transcript variant S, mRNA. |
| 2030309 | SERPING1 | 710 | Homo sapiens serpin peptidase inhibitor, clade G (C1 |
| | | | inhibitor), member 1 (SERPING1), transcript variant 2, |
| | | | mRNA. |
| 6770707 | SQRDL | 58472 | Homo sapiens sulfide quinone reductase-like (yeast) |
| | | | (SQRDL), mRNA. |
| 4860438 | DMXL2 | 23312 | Homo sapiens Dmx-like 2 (DMXL2), mRNA. |
| 6380338 | POLB | 5423 | Homo sapiens polymerase (DNA directed), beta (POLB), |
| | | | mRNA. |
| 3170273 | FER1L3 | 26509 | Homo sapiens fer-1-like 3, myoferlin (C. elegans) |
| | | | (FER1L3), transcript variant 2, mRNA. |
| 4230112 | LBH | 81606 | PREDICTED: Homo sapiens hypothetical protein |
| | | | DKFZp566J091 (LBH), mRNA. |
| 580343 | ZNF540 | 163255 | Homo sapiens zinc finger protein 540 (ZNF540), mRNA. |
| 7400743 | KIAA1618 | 57714 | Homo sapiens KIAA1618 (KIAA1618), mRNA. |
| 110338 | XRN1 | 54464 | Homo sapiens 5′-3′ exoribonuclease 1 (XRN1), transcript |
| | | | variant 2, mRNA. |
| 3890441 | | | UI-H-EU0-azk-k-10-0-UI.s1 NCI_CGAP_Car1 Homo |
| | | | sapiens cDNA clone IMAGE: 5849985 3, mRNA |
| | | | sequence |
| 4120561 | LIMK2 | 3985 | Homo sapiens LIM domain kinase 2 (LIMK2), transcript |
| | | | variant 1, mRNA. |
| 1510255 | LOC648164 | 648164 | PREDICTED: Homo sapiens similar to germinal histone |
| | | | H4 gene, transcript variant 1 (LOC648164), mRNA. |
| 3360491 | MCTP1 | 79772 | Homo sapiens multiple C2 domains, transmembrane 1 |
| | | | (MCTP1), transcript variant L, mRNA. |
| 3450452 | KIF1B | 23095 | Homo sapiens kinesin family member 1B (KIF1B), |
| | | | transcript variant 1, mRNA. |
| 7320546 | LAMP3 | 27074 | Homo sapiens lysosomal-associated membrane protein 3 |
| | | | (LAMP3), mRNA. |
| 2680050 | SIRPG | 55423 | Homo sapiens signal-regulatory protein gamma (SIRPG), |
| | | | transcript variant 2, mRNA. |
| 7560520 | | | Homo sapiens cDNA FLJ41813 fis, clone NT2RI2011450 |
| 450424 | CXCR7 | 57007 | Homo sapiens chemokine (C—X—C motif) receptor 7 |
| | | | (CXCR7), mRNA. |
| 5270403 | | | Homo sapiens cDNA FLJ20012 fis, clone ADKA03438 |
| 5360156 | IFITM1 | 8519 | Homo sapiens interferon induced transmembrane protein 1 |
| | | | (9-27) (IFITM1), mRNA. |
| 3830228 | GPR109A | 338442 | Homo sapiens G protein-coupled receptor 109A |
| | | | (GPR109A), mRNA. |
| 70431 | LOC650799 | 650799 | PREDICTED: Homo sapiens similar to Ig lambda chain |
| | | | V-I region BL2 precursor (LOC650799), mRNA. |
| 2510220 | IFI35 | 3430 | Homo sapiens interferon-induced protein 35 (IFI35), |
| | | | mRNA. |
| 6400176 | IRF7 | 3665 | Homo sapiens interferon regulatory factor 7 (IRF7), |
| | | | transcript variant b, mRNA. |
| 1470382 | IRF7 | 3665 | Homo sapiens interferon regulatory factor 7 (IRF7), |
| | | | transcript variant b, mRNA. |
| 5310445 | KREMEN1 | 83999 | Homo sapiens kringle containing transmembrane protein 1 |
| | | | (KREMEN1), transcript variant 4, mRNA. |
| 3390301 | KREMEN1 | 83999 | Homo sapiens kringle containing transmembrane protein 1 |
| | | | (KREMEN1), transcript variant 4, mRNA. |
| 5310754 | VNN1 | 8876 | Homo sapiens vanin 1 (VNN1), mRNA. |
| 6840035 | GBP1 | 2633 | Homo sapiens guanylate binding protein 1, interferon- |
| | | | inducible, 67 kDa (GBP1), mRNA. |
| 2190148 | GBP1 | 2633 | Homo sapiens guanylate binding protein 1, interferon- |
| | | | inducible, 67 kDa (GBP1), mRNA. |
| 4260368 | UBE2C | 11065 | Homo sapiens ubiquitin-conjugating enzyme E2C |
| | | | (UBE2C), transcript variant 3, mRNA. |
| 240053 | GCH1 | 2643 | Homo sapiens GTP cyclohydrolase 1 (GCH1), transcript |
| | | | variant 4, mRNA. |
| 3830349 | IL7R | 3575 | Homo sapiens interleukin 7 receptor (IL7R), mRNA. |
| 2510082 | GCET2 | 257144 | Homo sapiens germinal center expressed transcript 2 |
| | | | (GCET2), transcript variant 1, mRNA. |
| 1260424 | ADAM7 | 8756 | Homo sapiens ADAM metallopeptidase domain 7 |
| | | | (ADAM7), mRNA. |
| 3800398 | FBXO6 | 26270 | Homo sapiens F-box protein 6 (FBXO6), mRNA. |
| 5900524 | LACTB | 114294 | Homo sapiens lactamase, beta (LACTB), nuclear gene |
| | | | encoding mitochondrial protein, transcript variant 2, |
| | | | mRNA. |
| 5220477 | DPY19L3 | 147991 | Homo sapiens dpy-19-like 3 (C. elegans) (DPY19L3), |
| | | | mRNA. |
| 460373 | LACTB | 114294 | Homo sapiens lactamase, beta (LACTB), nuclear gene |
| | | | encoding mitochondrial protein, transcript variant 1, |
| | | | mRNA. |
| 6220332 | SLC6A12 | 6539 | Homo sapiens solute carrier family 6 (neurotransmitter |
| | | | transporter, betaine/GABA), member 12 (SLC6A12), |
| | | | mRNA. |
| 4890270 | LY6E | 4061 | Homo sapiens lymphocyte antigen 6 complex, locus E |
| | | | (LY6E), mRNA. |
| 7610563 | SP4 | 6671 | Homo sapiens Sp4 transcription factor (SP4), mRNA. |
| 4850132 | | | Homo sapiens T cell receptor alpha variable 20, mRNA |
| | | | (cDNA clone MGC: 34712 IMAGE: 5201547), complete |
| | | | cds |
| 1570129 | TRAFD1 | 10906 | Homo sapiens TRAF-type zinc finger domain containing |
| | | | 1 (TRAFD1), mRNA. |
| 1260228 | CLEC5A | 23601 | Homo sapiens C-type lectin domain family 5, member A |
| | | | (CLEC5A), mRNA. |
| 6510026 | IL23A | 51561 | Homo sapiens interleukin 23, alpha subunit p19 (IL23A), |
| | | | mRNA. |
| 2650279 | LOC643695 | 643695 | PREDICTED: Homo sapiens hypothetical protein |
| | | | LOC643695 (LOC643695), mRNA. |
| 6200577 | CREB5 | 9586 | Homo sapiens cAMP responsive element binding protein |
| | | | 5 (CREB5), transcript variant 1, mRNA. |
| 4730059 | BATF2 | 116071 | Homo sapiens basic leucine zipper transcription factor, |
| | | | ATF-like 2 (BATF2), mRNA. |
| 840068 | C3AR1 | 719 | Homo sapiens complement component 3a receptor 1 |
| | | | (C3AR1), mRNA. |
| 2070114 | CLEC4D | 338339 | Homo sapiens C-type lectin domain family 4, member D |
| | | | (CLEC4D), mRNA. |
| 7510647 | CACNA1E | 777 | Homo sapiens calcium channel, voltage-dependent, R |
| | | | type, alpha 1E subunit (CACNA1E), mRNA. |
| 1510170 | NLRP2 | 55655 | Homo sapiens NLR family, pyrin domain containing 2 |
| | | | (NLRP2), mRNA. |
| 580332 | SLC41A1 | 254428 | Homo sapiens solute carrier family 41, member 1 |
| | | | (SLC41A1), mRNA. |
| 2710520 | ATPBD4 | 89978 | Homo sapiens ATP binding domain 4 (ATPBD4), mRNA. |
| 2120328 | | | 17g11 Human retina cDNA randomly primed sublibrary |
| | | | Homo sapiens cDNA, mRNA sequence |
| 730528 | PLAUR | 5329 | Homo sapiens plasminogen activator, urokinase receptor |
| | | | (PLAUR), transcript variant 1, mRNA. |
| 830750 | NCF1B | 654816 | Homo sapiens neutrophil cytosolic factor 1B pseudogene |
| | | | (NCF1B), non-coding RNA. |
| 2970286 | CEACAM1 | 634 | Homo sapiens carcinoembryonic antigen-related cell |
| | | | adhesion molecule 1 (biliary glycoprotein) (CEACAM1), |
| | | | transcript variant 2, mRNA. |
| 5700753 | CEACAM1 | 634 | Homo sapiens carcinoembryonic antigen-related cell |
| | | | adhesion molecule 1 (biliary glycoprotein) (CEACAM1), |
| | | | transcript variant 2, mRNA. |
| 1820725 | LRRK2 | 120892 | Homo sapiens leucine-rich repeat kinase 2 (LRRK2), |
| | | | mRNA. |
| 3520291 | LRRK2 | 120892 | Homo sapiens leucine-rich repeat kinase 2 (LRRK2), |
| | | | mRNA. |
| 5260113 | MFSD7 | 84179 | PREDICTED: Homo sapiens major facilitator superfamily |
| | | | domain containing 7 (MFSD7), mRNA. |
| 3940477 | TAP2 | 6891 | Homo sapiens transporter 2, ATP-binding cassette, sub- |
| | | | family B (MDR/TAP) (TAP2), transcript variant 1, |
| | | | mRNA. |
| 3460053 | SIPA1L2 | 57568 | Homo sapiens signal-induced proliferation-associated 1 |
| | | | like 2 (SIPA1L2), mRNA. |
| 1240270 | LMTK2 | 22853 | Homo sapiens lemur tyrosine kinase 2 (LMTK2), mRNA. |
| 6420239 | CYFIP1 | 23191 | Homo sapiens cytoplasmic FMR1 interacting protein 1 |
| | | | (CYFIP1), transcript variant 2, mRNA. |
| 6280609 | TSGA14 | 95681 | Homo sapiens testis specific, 14 (TSGA14), mRNA. |
| 4570358 | CNTNAP1 | 8506 | Homo sapiens contactin associated protein 1 (CNTNAP1), |
| | | | mRNA. |
| 1510026 | FLVCR2 | 55640 | Homo sapiens feline leukemia virus subgroup C cellular |
| | | | receptor family, member 2 (FLVCR2), mRNA. |
| 6510129 | CCDC149 | 91050 | Homo sapiens coiled-coil domain containing 149 |
| | | | (CCDC149), mRNA. |
| 3420593 | LMNB1 | 4001 | Homo sapiens lamin B1 (LMNB1), mRNA. |
| 1940180 | PSMB9 | 5698 | Homo sapiens proteasome (prosome, macropain) subunit, |
| | | | beta type, 9 (large multifunctional peptidase 2) (PSMB9), |
| | | | transcript variant 1, mRNA. |
| 4780128 | ATF3 | 467 | Homo sapiens activating transcription factor 3 (ATF3), |
| | | | transcript variant 4, mRNA. |
| 5050347 | CD5 | 921 | Homo sapiens CD5 molecule (CD5), mRNA. |
| 160148 | AK3L1 | 205 | Homo sapiens adenylate kinase 3-like 1 (AK3L1), nuclear |
| | | | gene encoding mitochondrial protein, transcript variant 6, |
| | | | mRNA. |
| 2640025 | HP | 3240 | Homo sapiens haptoglobin (HP), mRNA. |
| 160019 | SORT1 | 6272 | Homo sapiens sortilin 1 (SORT1), mRNA. |
| 2060440 | MAFB | 9935 | Homo sapiens v-maf musculoaponeurotic fibrosarcoma |
| | | | oncogene homolog B (avian) (MAFB), mRNA. |
| 4810707 | | | DB376622 PLACE1 Homo sapiens cDNA clone |
| | | | PLACE1006439 3, mRNA sequence |
| 2570019 | C11orf82 | 220042 | Homo sapiens chromosome 11 open reading frame 82 |
| | | | (C11orf82), mRNA. |
| 6330471 | BLK | 640 | Homo sapiens B lymphoid tyrosine kinase (BLK), mRNA. |
| 3440360 | ZNF438 | 220929 | Homo sapiens zinc finger protein 438 (ZNF438), mRNA. |
| 4850168 | SLC22A4 | 6583 | Homo sapiens solute carrier family 22 (organic |
| | | | cation/ergothioneine transporter), member 4 (SLC22A4), |
| | | | mRNA. |
| 130661 | LOC650795 | 650795 | PREDICTED: Homo sapiens similar to T-cell receptor |
| | | | alpha chain V region PY14 precursor (LOC650795), |
| | | | mRNA. |
| 4260386 | CTSL1 | 1514 | Homo sapiens cathepsin L1 (CTSL1), transcript variant 2, |
| | | | mRNA. |
| 6480059 | ACTA2 | 59 | Homo sapiens actin, alpha 2, smooth muscle, aorta |
| | | | (ACTA2), mRNA. |
| 3130669 | SATB1 | 6304 | Homo sapiens SATB homeobox 1 (SATB1), mRNA. |
| 6040653 | TRIM6 | 117854 | Homo sapiens tripartite motif-containing 6 (TRIM6), |
| | | | transcript variant 2, mRNA. |
| 6100136 | CASP4 | 837 | Homo sapiens caspase 4, apoptosis-related cysteine |
| | | | peptidase (CASP4), transcript variant gamma, mRNA. |
| 6040398 | SLC22A4 | 6583 | Homo sapiens solute carrier family 22 (organic |
| | | | cation/ergothioneine transporter), member 4 (SLC22A4), |
| | | | mRNA. |
| 670279 | ZNF485 | 220992 | Homo sapiens zinc finger protein 485 (ZNF485), mRNA. |
| 6420750 | TLR2 | 7097 | Homo sapiens toll-like receptor 2 (TLR2), mRNA. |
| 5910300 | RAB24 | 53917 | Homo sapiens RAB24, member RAS oncogene family |
| | | | (RAB24), transcript variant 1, mRNA. |
| 1580358 | WDFY3 | 23001 | Homo sapiens WD repeat and FYVE domain containing 3 |
| | | | (WDFY3), transcript variant 1, mRNA. |
| 1450414 | MLKL | 197259 | PREDICTED: Homo sapiens mixed lineage kinase |
| | | | domain-like (MLKL), mRNA. |
| 5890564 | ANKDD1A | 348094 | Homo sapiens ankyrin repeat and death domain containing |
| | | | 1A (ANKDD1A), mRNA. |
| 6480326 | MCTP1 | 79772 | Homo sapiens multiple C2 domains, transmembrane 1 |
| | | | (MCTP1), transcript variant S, mRNA. |
| 540091 | STEAP4 | 79689 | Homo sapiens STEAP family member 4 (STEAP4), |
| | | | mRNA. |
| 3420373 | SOD2 | 6648 | Homo sapiens superoxide dismutase 2, mitochondrial |
| | | | (SOD2), nuclear gene encoding mitochondrial protein, |
| | | | transcript variant 3, mRNA. |
| 6250497 | FBXL5 | 26234 | Homo sapiens F-box and leucine-rich repeat protein 5 |
| | | | (FBXL5), transcript variant 1, mRNA. |
| 2230730 | SRBD1 | 55133 | Homo sapiens S1 RNA binding domain 1 (SRBD1), |
| | | | mRNA. |
| 2030348 | | | AGENCOURT_14538823 NIH_MGC_191 Homo sapiens |
| | | | cDNA clone IMAGE: 30418790 5, mRNA sequence |
| 6650333 | LOC653867 | 653867 | PREDICTED: Homo sapiens similar to Occludin |
| | | | (LOC653867), mRNA. |
| 5220026 | KRT73 | 319101 | Homo sapiens keratin 73 (KRT73), mRNA. |
| 3360431 | ZAK | 51776 | Homo sapiens sterile alpha motif and leucine zipper |
| | | | containing kinase AZK (ZAK), transcript variant 1, |
| | | | mRNA. |
| 150333 | | | Homo sapiens (clone 5) macronuclear mRNA |
| 5390328 | KCNJ15 | 3772 | Homo sapiens potassium inwardly-rectifying channel, |
| | | | subfamily J, member 15 (KCNJ15), transcript variant 1, |
| | | | mRNA. |
| 1050215 | KCNJ15 | 3772 | Homo sapiens potassium inwardly-rectifying channel, |
| | | | subfamily J, member 15 (KCNJ15), transcript variant 1, |
| | | | mRNA. |
| 4570368 | CD86 | 942 | Homo sapiens CD86 antigen (CD28 antigen ligand 2, B7- |
| | | | 2 antigen) (CD86), transcript variant 2, mRNA. |
| 3450180 | OAS1 | 4938 | Homo sapiens 2′,5′-oligoadenylate synthetase 1, 40/46 kDa |
| | | | (OAS1), transcript variant 2, mRNA. |
| 6020097 | SERPINA1 | 5265 | Homo sapiens serpin peptidase inhibitor, clade A (alpha-1 |
| | | | antiproteinase, antitrypsin), member 1 (SERPINA1), |
| | | | transcript variant 2, mRNA. |
| 1240037 | DKFZP564O0823 | 25849 | Homo sapiens DKFZP564O0823 protein |
| | | | (DKFZP564O0823), mRNA. |
| 620403 | LOC400759 | 400759 | Homo sapiens similar to Interferon-induced guanylate- |
| | | | binding protein 1 (GTP-binding protein 1) (Guanine |
| | | | nucleotide-binding protein 1) (HuGBP-1) (LOC400759) |
| | | | on chromosome 1. |
| 6280470 | LOC728519 | 728519 | PREDICTED: Homo sapiens similar to Baculoviral IAP |
| | | | repeat-containing protein 1 (Neuronal apoptosis inhibitory |
| | | | protein) (LOC728519), mRNA. |
| 2630577 | CAV2 | 858 | Homo sapiens caveolin 2 (CAV2), transcript variant 1, |
| | | | mRNA. |
| 6560079 | SUCNR1 | 56670 | Homo sapiens succinate receptor 1 (SUCNR1), mRNA. |
| 5570114 | GADD45G | 10912 | Homo sapiens growth arrest and DNA-damage-inducible, |
| | | | gamma (GADD45G), mRNA. |
| 4830010 | USP30 | 84749 | Homo sapiens ubiquitin specific peptidase 30 (USP30), |
| | | | mRNA. |
| 6940369 | SIPA1L1 | 26037 | Homo sapiens signal-induced proliferation-associated 1 |
| | | | like 1 (SIPA1L1), mRNA. |
| 5490743 | KIF1B | 23095 | Homo sapiens kinesin family member 1B (KIF1B), |
| | | | transcript variant 2, mRNA. |
| 2360719 | IRAK3 | 11213 | Homo sapiens interleukin-1 receptor-associated kinase 3 |
| | | | (IRAK3), mRNA. |
| 1050020 | NLRC4 | 58484 | Homo sapiens NLR family, CARD domain containing 4 |
| | | | (NLRC4), mRNA. |
| 3390121 | CASP5 | 838 | Homo sapiens caspase 5, apoptosis-related cysteine |
| | | | peptidase (CASP5), mRNA. |
| 5890386 | LOC642103 | 642103 | PREDICTED: Homo sapiens similar to Maltase- |
| | | | glucoamylase, intestinal (LOC642103), mRNA. |
| 4760433 | C16orf7 | 9605 | Homo sapiens chromosome 16 open reading frame 7 |
| | | | (C16orf7), mRNA. |
| 1110592 | EBF1 | 1879 | Homo sapiens early B-cell factor 1 (EBF1), mRNA. |
| 5050193 | RLTPR | 146206 | Homo sapiens RGD motif, leucine rich repeats, |
| | | | tropomodulin domain and proline-rich containing |
| | | | (RLTPR), mRNA. |
| 2320689 | LOC653610 | 653610 | PREDICTED: Homo sapiens similar to Histone H2A.o |
| | | | (H2A/o) (H2A.2) (H2a-615) (LOC653610), mRNA. |
| 5130767 | MAPK14 | 1432 | Homo sapiens mitogen-activated protein kinase 14 |
| | | | (MAPK14), transcript variant 1, mRNA. |
| 5550367 | LTF | 4057 | Homo sapiens lactotransferrin (LTF), mRNA. |
| 840685 | IL1B | 3553 | Homo sapiens interleukin 1, beta (IL1B), mRNA. |
| 6580484 | MOV10 | 4343 | Homo sapiens Mov10, Moloney leukemia virus 10, |
| | | | homolog (mouse) (MOV10), mRNA. |
| 1400484 | LDOC1 | 23641 | Homo sapiens leucine zipper, down-regulated in cancer 1 |
| | | | (LDOC1), mRNA. |
| 130753 | AIM2 | 9447 | Homo sapiens absent in melanoma 2 (AIM2), mRNA. |
| 1260270 | AIM2 | 9447 | Homo sapiens absent in melanoma 2 (AIM2), mRNA. |
| 6860246 | GRAMD1C | 54762 | Homo sapiens GRAM domain containing 1C |
| | | | (GRAMD1C), mRNA. |
| 1340500 | CLEC12A | 160364 | Homo sapiens C-type lectin domain family 12, member A |
| | | | (CLEC12A), transcript variant 1, mRNA. |
| 3780358 | CLEC12A | 160364 | Homo sapiens C-type lectin domain family 12, member A |
| | | | (CLEC12A), transcript variant 1, mRNA. |
| 2230044 | DOCK9 | 23348 | Homo sapiens dedicator of cytokinesis 9 (DOCK9), |
| | | | mRNA. |
| 3990433 | ADAP2 | 55803 | Homo sapiens ArfGAP with dual PH domains 2 |
| | | | (ADAP2), mRNA. |
| 6280594 | MMP8 | 4317 | Homo sapiens matrix metallopeptidase 8 (neutrophil |
| | | | collagenase) (MMP8), mRNA. |
| 380070 | RPS23 | 6228 | Homo sapiens ribosomal protein S23 (RPS23), mRNA. |
| 5390427 | NAIP | 4671 | Homo sapiens NLR family, apoptosis inhibitory protein |
| | | | (NAIP), transcript variant 1, mRNA. |
| 5340240 | NAIP | 4671 | Homo sapiens NLR family, apoptosis inhibitory protein |
| | | | (NAIP), transcript variant 1, mRNA. |
| 7550358 | NELL2 | 4753 | Homo sapiens NEL-like 2 (chicken) (NELL2), mRNA. |
| 4860600 | MAPK14 | 1432 | Homo sapiens mitogen-activated protein kinase 14 |
| | | | (MAPK14), transcript variant 3, mRNA. |
| 6110753 | NBN | 4683 | Homo sapiens nibrin (NBN), transcript variant 2, mRNA. |
| 1010360 | GCH1 | 2643 | Homo sapiens GTP cyclohydrolase 1 (GCH1), transcript |
| | | | variant 3, mRNA. |
| 1170653 | PDGFA | 5154 | Homo sapiens platelet-derived growth factor alpha |
| | | | polypeptide (PDGFA), transcript variant 2, mRNA. |
| 2680072 | OLFM1 | 10439 | Homo sapiens olfactomedin 1 (OLFM1), transcript variant |
| | | | 1, mRNA. |
| 1260021 | ZBP1 | 81030 | Homo sapiens Z-DNA binding protein 1 (ZBP1), mRNA. |
| 4010270 | LOC440731 | 440731 | PREDICTED: Homo sapiens hypothetical LOC440731, |
| | | | transcript variant 2 (LOC440731), mRNA. |
| 60470 | STX11 | 8676 | Homo sapiens syntaxin 11 (STX11), mRNA. |
| 5910019 | C1QB | 713 | Homo sapiens complement component 1, q |
| | | | subcomponent, B chain (C1QB), mRNA. |
| 4890707 | PVRL2 | 5819 | Homo sapiens poliovirus receptor-related 2 (herpesvirus |
| | | | entry mediator B) (PVRL2), transcript variant alpha, |
| | | | mRNA. |
| 6580717 | ZMYND15 | 84225 | Homo sapiens zinc finger, MYND-type containing 15 |
| | | | (ZMYND15), mRNA. |
| 5360093 | SECTM1 | 6398 | Homo sapiens secreted and transmembrane 1 (SECTM1), |
| | | | mRNA. |
| 3890400 | CXCR5 | 643 | Homo sapiens chemokine (C—X—C motif) receptor 5 |
| | | | (CXCR5), transcript variant 2, mRNA. |
| 2710709 | FCGR1B | 2210 | Homo sapiens Fc fragment of IgG, high affinity Ib, |
| | | | receptor (CD64) (FCGR1B), transcript variant 1, mRNA. |
| 1010672 | NR3C2 | 4306 | Homo sapiens nuclear receptor subfamily 3, group C, |
| | | | member 2 (NR3C2), mRNA. |
| 4040037 | EBI2 | 1880 | Homo sapiens Epstein-Barr virus induced gene 2 |
| | | | (lymphocyte-specific G protein-coupled receptor) (EBI2), |
| | | | mRNA. |
| 4490520 | EBI2 | 1880 | Homo sapiens Epstein-Barr virus induced gene 2 |
| | | | (lymphocyte-specific G protein-coupled receptor) (EBI2), |
| | | | mRNA. |
| 2120746 | | | EST10086 human nasopharynx Homo sapiens cDNA, |
| | | | mRNA sequence |
| 1190167 | LOC152195 | 152195 | Homo sapiens hypothetical protein LOC152195 |
| | | | (LOC152195), mRNA. |
| 4210167 | CIITA | 4261 | Homo sapiens class II, major histocompatibility complex, |
| | | | transactivator (CIITA), mRNA. |
| 830440 | TLR5 | 7100 | Homo sapiens toll-like receptor 5 (TLR5), mRNA. |
| 1260482 | GZMK | 3003 | Homo sapiens granzyme K (granzyme 3; tryptase II) |
| | | | (GZMK), mRNA. |
| 3930368 | INCA | 440068 | Homo sapiens inhibitory caspase recruitment domain |
| | | | (CARD) protein (INCA), mRNA. |
| 3830138 | THRA | 7067 | Homo sapiens thyroid hormone receptor, alpha |
| | | | (erythroblastic leukemia viral (v-erb-a) oncogene |
| | | | homolog, avian) (THRA), transcript variant 2, mRNA. |
| 7050056 | SRPK1 | 6732 | Homo sapiens SFRS protein kinase 1 (SRPK1), mRNA. |
| 360132 | LHFPL2 | 10184 | Homo sapiens lipoma HMGIC fusion partner-like 2 |
| | | | (LHFPL2), mRNA. |
| 1050025 | CR1 | 1378 | Homo sapiens complement component (3b/4b) receptor 1 |
| | | | (Knops blood group) (CR1), transcript variant F, mRNA. |
| 4640184 | KIAA1026 | 23254 | Homo sapiens kazrin (KIAA1026), transcript variant B, |
| | | | mRNA. |
| 1990427 | KIAA1026 | 23254 | Homo sapiens kazrin (KIAA1026), transcript variant B, |
| | | | mRNA. |
| 3990435 | | | BX282075 NIH_MGC_120 Homo sapiens cDNA clone |
| | | | IMAGp998M2011561; IMAGE: 5223355, mRNA |
| | | | sequence |
| 3840215 | FAM102A | 399665 | Homo sapiens family with sequence similarity 102, |
| | | | member A (FAM102A), transcript variant 1, mRNA. |
| 1850546 | FAM102A | 399665 | Homo sapiens family with sequence similarity 102, |
| | | | member A (FAM102A), transcript variant 1, mRNA. |
| 2340632 | INADL | 10207 | Homo sapiens InaD-like (Drosophila) (INADL), transcript |
| | | | variant 4, mRNA. |
| 6450326 | CHMP5 | 51510 | Homo sapiens chromatin modifying protein 5 (CHMP5), |
| | | | mRNA. |
| 6420594 | TIFA | 92610 | Homo sapiens TRAF-interacting protein with forkhead- |
| | | | associated domain (TIFA), mRNA. |
| 1300192 | ARL4C | 10123 | Homo sapiens ADP-ribosylation factor-like 4C (ARL4C), |
| | | | mRNA. |
| 3290674 | PCNXL2 | 80003 | Homo sapiens pecanex-like 2 (Drosophila) (PCNXL2), |
| | | | transcript variant 1, mRNA. |
| 6650482 | EPHA4 | 2043 | Homo sapiens EPH receptor A4 (EPHA4), mRNA. |
| 4640561 | | | Homo sapiens T cell antigen receptor alpha chain |
| | | | (TCRVA2) mRNA, complete cds |
| 1070367 | C19orf59 | 199675 | Homo sapiens chromosome 19 open reading frame 59 |
| | | | (C19orf59), mRNA. |
| 4260017 | CDK5RAP2 | 55755 | Homo sapiens CDK5 regulatory subunit associated protein |
| | | | 2 (CDK5RAP2), transcript variant 2, mRNA. |
| 3890609 | PLSCR1 | 5359 | Homo sapiens phospholipid scramblase 1 (PLSCR1), |
| | | | mRNA. |
| 5550445 | FLJ22795 | 80154 | Homo sapiens hypothetical protein FLJ22795 (FLJ22795), |
| | | | mRNA. |
| 870202 | TNFSF10 | 8743 | Homo sapiens tumor necrosis factor (ligand) superfamily, |
| | | | member 10 (TNFSF10), mRNA. |
| 3780382 | DKFZp761E198 | 91056 | Homo sapiens DKFZp761E198 protein |
| | | | (DKFZp761E198), mRNA. |
| 4150692 | PARP14 | 54625 | Homo sapiens poly (ADP-ribose) polymerase family, |
| | | | member 14 (PARP14), mRNA. |
| 5700735 | PARP9 | 83666 | Homo sapiens poly (ADP-ribose) polymerase family, |
| | | | member 9 (PARP9), mRNA. |
| 3140707 | PARP9 | 83666 | Homo sapiens poly (ADP-ribose) polymerase family, |
| | | | member 9 (PARP9), mRNA. |
| 780035 | SYTL2 | 54843 | Homo sapiens synaptotagmin-like 2 (SYTL2), transcript |
| | | | variant a, mRNA. |
| 6650064 | SIGLEC5 | 8778 | Homo sapiens sialic acid binding Ig-like lectin 5 |
| | | | (SIGLEC5), mRNA. |
| 2970092 | SIGLEC5 | 8778 | Homo sapiens sialic acid binding Ig-like lectin 5 |
| | | | (SIGLEC5), mRNA. |
| 5290292 | DHRS9 | 10170 | Homo sapiens dehydrogenase/reductase (SDR family) |
| | | | member 9 (DHRS9), transcript variant 2, mRNA. |
| 990239 | KCNE3 | 10008 | Homo sapiens potassium voltage-gated channel, Isk- |
| | | | related family, member 3 (KCNE3), mRNA. |
| 150240 | TRIMP1 | 117852 | Homo sapiens tripartite motif-containing pseudogene 1 |
| | | | (TRIMP1), non-coding RNA. |
| 4480270 | | | DA830074 PLACE1 Homo sapiens cDNA clone |
| | | | PLACE1004374 5, mRNA sequence |
| 3060612 | HK3 | 3101 | Homo sapiens hexokinase 3 (white cell) (HK3), nuclear |
| | | | gene encoding mitochondrial protein, mRNA. |
| 5810047 | F2RL1 | 2150 | Homo sapiens coagulation factor II (thrombin) receptor- |
| | | | like 1 (F2RL1), mRNA. |
| 4150008 | LYST | 1130 | Homo sapiens lysosomal trafficking regulator (LYST), |
| | | | mRNA. |
| 2120612 | SGPP2 | 130367 | PREDICTED: Homo sapiens sphingosine-1-phosphate |
| | | | phosphotase 2 (SGPP2), mRNA. |
| 2470601 | IL1RN | 3557 | Homo sapiens interleukin 1 receptor antagonist (IL1RN), |
| | | | transcript variant 1, mRNA. |
| 990768 | OAS3 | 4940 | Homo sapiens 2′-5′-oligoadenylate synthetase 3, 100 kDa |
| | | | (OAS3), mRNA. |
| 6900521 | PHTF1 | 10745 | Homo sapiens putative homeodomain transcription factor |
| | | | 1 (PHTF1), mRNA. |
| 1300706 | MTHFD2 | 10797 | Homo sapiens methylenetetrahydrofolate dehydrogenase |
| | | | (NADP+ dependent) 2, methenyltetrahydrofolate |
| | | | cyclohydrolase (MTHFD2), nuclear gene encoding |
| | | | mitochondrial protein, transcript variant 1, mRNA. |
| 5340767 | CEACAM1 | 634 | Homo sapiens carcinoembryonic antigen-related cell |
| | | | adhesion molecule 1 (biliary glycoprotein) (CEACAM1), |
| | | | transcript variant 1, mRNA. |
| 450189 | XAF1 | 54739 | Homo sapiens XIAP associated factor 1 (XAF1), |
| | | | transcript variant 2, mRNA. |
| 7610440 | XAF1 | 54739 | Homo sapiens XIAP associated factor 1 (XAF1), |
| | | | transcript variant 2, mRNA. |
| 3060497 | ING5 | 84289 | Homo sapiens inhibitor of growth family, member 5 |
| | | | (ING5), mRNA. |
| 1940373 | ZNF831 | 128611 | Homo sapiens zinc finger protein 831 (ZNF831), mRNA. |
| 2710646 | MSRB2 | 22921 | Homo sapiens methionine sulfoxide reductase B2 |
| | | | (MSRB2), mRNA. |
| 4890327 | IFIT5 | 24138 | Homo sapiens interferon-induced protein with |
| | | | tetratricopeptide repeats 5 (IFIT5), mRNA. |
| 110372 | CSTA | 1475 | Homo sapiens cystatin A (stefin A) (CSTA), mRNA. |
| 6380411 | IKZF3 | 22806 | Homo sapiens IKAROS family zinc finger 3 (Aiolos) |
| | | | (IKZF3), transcript variant 1, mRNA. |
| 3710152 | TRIM5 | 85363 | Homo sapiens tripartite motif-containing 5 (TRIM5), |
| | | | transcript variant gamma, mRNA. |
| 4040386 | APOL1 | 8542 | Homo sapiens apolipoprotein L, 1 (APOL1), transcript |
| | | | variant 2, mRNA. |
| 2000300 | IFI30 | 10437 | Homo sapiens interferon, gamma-inducible protein 30 |
| | | | (IFI30), mRNA. |
| 380050 | RHBDF2 | 79651 | Homo sapiens rhomboid 5 homolog 2 (Drosophila) |
| | | | (RHBDF2), transcript variant 1, mRNA. |
| 4780427 | KIAA0082 | 23070 | Homo sapiens KIAA0082 (KIAA0082), mRNA. |
| 4220184 | | | PREDICTED: Homo sapiens LOC440151 (LOC440151), |
| | | | mRNA |
| 2570402 | KIF5C | 3800 | Homo sapiens kinesin family member 5C (KIF5C), |
| | | | mRNA. |
| 3310341 | FYB | 2533 | Homo sapiens FYN binding protein (FYB-120/130) |
| | | | (FYB), transcript variant 1, mRNA. |
| 5560193 | BCAS4 | 55653 | Homo sapiens breast carcinoma amplified sequence 4 |
| | | | (BCAS4), transcript variant 1, mRNA. |
| 1240674 | DENND1A | 57706 | Homo sapiens DENN/MADD domain containing 1A |
| | | | (DENND1A), transcript variant 1, mRNA. |
| 6370487 | TLR8 | 51311 | Homo sapiens toll-like receptor 8 (TLR8), transcript |
| | | | variant 1, mRNA. |
| 3310376 | TIMM10 | 26519 | Homo sapiens translocase of inner mitochondrial |
| | | | membrane 10 homolog (yeast) (TIMM10), nuclear gene |
| | | | encoding mitochondrial protein, mRNA. |
| 7330392 | TAP1 | 6890 | Homo sapiens transporter 1, ATP-binding cassette, sub- |
| | | | family B (MDR/TAP) (TAP1), mRNA. |
| 3460608 | HORMAD1 | 84072 | Homo sapiens HORMA domain containing 1 |
| | | | (HORMAD1), mRNA. |
| 2480360 | LOC653197 | 653197 | PREDICTED: Homo sapiens similar to hypothetical |
| | | | protein FLJ33915, transcript variant 1 (LOC653197), |
| | | | mRNA. |
| 6770347 | LOC643720 | 643720 | PREDICTED: Homo sapiens similar to Protein C21orf109 |
| | | | (LOC643720), mRNA. |
| 7380221 | LOC26010 | 26010 | Homo sapiens viral DNA polymerase-transactivated |
| | | | protein 6 (LOC26010), transcript variant 2, mRNA. |
| 1570358 | APOL2 | 23780 | Homo sapiens apolipoprotein L, 2 (APOL2), transcript |
| | | | variant beta, mRNA. |
| 5700754 | APOL2 | 23780 | Homo sapiens apolipoprotein L, 2 (APOL2), transcript |
| | | | variant beta, mRNA. |
| 4150100 | PASK | 23178 | Homo sapiens PAS domain containing serine/threonine |
| | | | kinase (PASK), mRNA. |
| 2140382 | PASK | 23178 | Homo sapiens PAS domain containing serine/threonine |
| | | | kinase (PASK), mRNA. |
| 5340170 | SNORD21 | 6083 | Homo sapiens small nucleolar RNA, C/D box 21 |
| | | | (SNORD21), non-coding RNA. |
| 6510707 | FER1L3 | 26509 | Homo sapiens fer-1-like 3, myoferlin (C. elegans) |
| | | | (FER1L3), transcript variant 1, mRNA. |
| 3830433 | | | BX096301 Soares retina N2b5HR Homo sapiens cDNA |
| | | | clone IMAGp998C16444, mRNA sequence |
| 870408 | IL15 | 3600 | Homo sapiens interleukin 15 (IL15), transcript variant 3, |
| | | | mRNA. |
| 1710553 | HSPA6 | 3310 | Homo sapiens heat shock 70 kDa protein 6 (HSP70B′) |
| | | | (HSPA6), mRNA. |
| 770253 | KIAA1618 | 57714 | PREDICTED: Homo sapiens KIAA1618 (KIAA1618), |
| | | | mRNA. |
| 1450400 | ZDHHC11 | 79844 | Homo sapiens zinc finger, DHHC-type containing 11 |
| | | | (ZDHHC11), mRNA. |
| 4290619 | CREB5 | 9586 | Homo sapiens cAMP responsive element binding protein |
| | | | 5 (CREB5), transcript variant 4, mRNA. |
| 3420026 | FAS | 355 | Homo sapiens Fas (TNF receptor superfamily, member 6) |
| | | | (FAS), transcript variant 7, mRNA. |
| 3360343 | RSAD2 | 91543 | Homo sapiens radical S-adenosyl methionine domain |
| | | | containing 2 (RSAD2), mRNA. |
| 2070020 | ZNF667 | 63934 | Homo sapiens zinc finger protein 667 (ZNF667), mRNA. |
| 450491 | CASP1 | 834 | Homo sapiens caspase 1, apoptosis-related cysteine |
| | | | peptidase (interleukin 1, beta, convertase) (CASP1), |
| | | | transcript variant delta, mRNA. |
| 7050382 | CASP1 | 834 | Homo sapiens caspase 1, apoptosis-related cysteine |
| | | | peptidase (interleukin 1, beta, convertase) (CASP1), |
| | | | transcript variant delta, mRNA. |
| 1050377 | PLVAP | 83483 | Homo sapiens plasmalemma vesicle associated protein |
| | | | (PLVAP), mRNA. |
| 2470215 | NDST1 | 3340 | Homo sapiens N-deacetylase/N-sulfotransferase (heparan |
| | | | glucosaminyl) 1 (NDST1), mRNA. |
| 1010246 | IFI6 | 2537 | Homo sapiens interferon, alpha-inducible protein 6 (IFI6), |
| | | | transcript variant 2, mRNA. |
| 1090390 | OAS1 | 4938 | Homo sapiens 2′,5′-oligoadenylate synthetase 1, 40/46 kDa |
| | | | (OAS1), transcript variant 3, mRNA. |
| 7040035 | OAS1 | 4938 | Homo sapiens 2′,5′-oligoadenylate synthetase 1, 40/46 kDa |
| | | | (OAS1), transcript variant 3, mRNA. |
| 2140020 | LIMK2 | 3985 | Homo sapiens LIM domain kinase 2 (LIMK2), transcript |
| | | | variant 2b, mRNA. |
| 3390048 | LIMK2 | 3985 | Homo sapiens LIM domain kinase 2 (LIMK2), transcript |
| | | | variant 2b, mRNA. |
| 5910632 | SMARCD3 | 6604 | Homo sapiens SWI/SNF related, matrix associated, actin |
| | | | dependent regulator of chromatin, subfamily d, member 3 |
| | | | (SMARCD3), transcript variant 2, mRNA. |
| 6770497 | PLAG1 | 5324 | Homo sapiens pleiomorphic adenoma gene 1 (PLAG1), |
| | | | mRNA. |
| 6100484 | TNNI2 | 7136 | Homo sapiens troponin I type 2 (skeletal, fast) (TNNI2), |
| | | | mRNA. |
| 3170136 | SAMD9L | 219285 | Homo sapiens sterile alpha motif domain containing 9-like |
| | | | (SAMD9L), mRNA. |
| 5050537 | OPLAH | 26873 | Homo sapiens 5-oxoprolinase (ATP-hydrolysing) |
| | | | (OPLAH), mRNA. |
| 3520167 | CD63 | 967 | Homo sapiens CD63 molecule (CD63), transcript variant |
| | | | 2, mRNA. |
| 4900747 | C11orf75 | 56935 | Homo sapiens chromosome 11 open reading frame 75 |
| | | | (C11orf75), mRNA. |
| 110592 | SUSD2 | 56241 | Homo sapiens sushi domain containing 2 (SUSD2), |
| | | | mRNA. |
| 1820114 | LILRB4 | 11006 | Homo sapiens leukocyte immunoglobulin-like receptor, |
| | | | subfamily B (with TM and ITIM domains), member 4 |
| | | | (LILRB4), transcript variant 2, mRNA. |
| 130519 | STAT2 | 6773 | Homo sapiens signal transducer and activator of |
| | | | transcription 2, 113 kDa (STAT2), mRNA. |
| 7570328 | STXBP4 | 252983 | Homo sapiens syntaxin binding protein 4 (STXBP4), |
| | | | mRNA. |
| 1240358 | RAB20 | 55647 | Homo sapiens RAB20, member RAS oncogene family |
| | | | (RAB20), mRNA. |
| 1990300 | SOCS1 | 8651 | Homo sapiens suppressor of cytokine signaling 1 |
| | | | (SOCS1), mRNA. |
| 460064 | DGKA | 1606 | Homo sapiens diacylglycerol kinase, alpha 80 kDa |
| | | | (DGKA), transcript variant 3, mRNA. |
| 7040477 | PLOD2 | 5352 | Homo sapiens procollagen-lysine, 2-oxoglutarate 5- |
| | | | dioxygenase 2 (PLOD2), transcript variant 2, mRNA. |
| 630315 | DHRS9 | 10170 | Homo sapiens dehydrogenase/reductase (SDR family) |
| | | | member 9 (DHRS9), transcript variant 1, mRNA. |
| 830403 | PSME2 | 5721 | Homo sapiens proteasome (prosome, macropain) activator |
| | | | subunit 2 (PA28 beta) (PSME2), mRNA. |
| 2900427 | P2RX5 | 5026 | Homo sapiens purinergic receptor P2X, ligand-gated ion |
| | | | channel, 5 (P2RX5), transcript variant 2, mRNA. |
| 2100196 | ISG15 | 9636 | Homo sapiens ISG15 ubiquitin-like modifier (ISG15), |
| | | | mRNA. |
| 3170601 | CLEC12A | 160364 | Homo sapiens C-type lectin domain family 12, member A |
| | | | (CLEC12A), transcript variant 2, mRNA. |
| 6280195 | LOC647104 | 647104 | PREDICTED: Homo sapiens hypothetical LOC647104 |
| | | | (LOC647104), mRNA. |
| 3290292 | LAP3 | 51056 | Homo sapiens leucine aminopeptidase 3 (LAP3), mRNA. |
| 7400328 | ZNF681 | 148213 | Homo sapiens zinc finger protein 681 (ZNF681), mRNA. |
| 4900435 | TNFSF13B | 10673 | Homo sapiens tumor necrosis factor (ligand) superfamily, |
| | | | member 13b (TNFSF13B), mRNA. |
| 4850592 | P2RY14 | 9934 | Homo sapiens purinergic receptor P2Y, G-protein |
| | | | coupled, 14 (P2RY14), transcript variant 2, mRNA. |
| 1340364 | P2RY14 | 9934 | Homo sapiens purinergic receptor P2Y, G-protein |
| | | | coupled, 14 (P2RY14), transcript variant 2, mRNA. |
| 6510170 | IFIT3 | 3437 | Homo sapiens interferon-induced protein with |
| | | | tetratricopeptide repeats 3 (IFIT3), mRNA. |
| 1500280 | IFIT3 | 3437 | Homo sapiens interferon-induced protein with |
| | | | tetratricopeptide repeats 3 (IFIT3), mRNA. |
| 4180544 | ROPN1L | 83853 | Homo sapiens ropporin 1-like (ROPN1L), mRNA. |
| 1440360 | GPR141 | 353345 | Homo sapiens G protein-coupled receptor 141 (GPR141), |
| | | | mRNA. |
| 5050647 | GPR141 | 353345 | Homo sapiens G protein-coupled receptor 141 (GPR141), |
| | | | mRNA. |
| 610358 | PGS1 | 9489 | Homo sapiens phosphatidylglycerophosphate synthase 1 |
| | | | (PGS1), mRNA. |
| 540390 | ALPK1 | 80216 | Homo sapiens alpha-kinase 1 (ALPK1), mRNA. |
| 7510386 | IL1RN | 3557 | Homo sapiens interleukin 1 receptor antagonist (IL1RN), |
| | | | transcript variant 4, mRNA. |
| 3180504 | KIAA1394 | 57571 | Homo sapiens KIAA1394 protein (KIAA1394), mRNA. |
| 1980356 | | | af01b06.s1 Human bone marrow stromal cells Homo |
| | | | sapiens cDNA clone IMAGE: 1027283 3, mRNA |
| | | | sequence |
| 4120053 | PFKFB3 | 5209 | Homo sapiens 6-phosphofructo-2-kinase/fructose-2,6- |
| | | | biphosphatase 3 (PFKFB3), mRNA. |
| 4150270 | ANKRD22 | 118932 | Homo sapiens ankyrin repeat domain 22 (ANKRD22), |
| | | | mRNA. |
| 50706 | CD40LG | 959 | Homo sapiens CD40 ligand (CD40LG), mRNA. |
| 3990673 | AFF1 | 4299 | Homo sapiens AF4/FMR2 family, member 1 (AFF1), |
| | | | mRNA. |
| 2140524 | HIST1H3D | 8351 | Homo sapiens histone cluster 1, H3d (HIST1H3D), |
| | | | mRNA. |
| 7610113 | SLC26A8 | 116369 | Homo sapiens solute carrier family 26, member 8 |
| | | | (SLC26A8), transcript variant 2, mRNA. |
| 5360626 | SLC26A8 | 116369 | Homo sapiens solute carrier family 26, member 8 |
| | | | (SLC26A8), transcript variant 2, mRNA. |
| 2070170 | UBE2L6 | 9246 | Homo sapiens ubiquitin-conjugating enzyme E2L 6 |
| | | | (UBE2L6), transcript variant 1, mRNA. |
| 7000368 | UBE2L6 | 9246 | Homo sapiens ubiquitin-conjugating enzyme E2L 6 |
| | | | (UBE2L6), transcript variant 1, mRNA. |
| 770088 | DTX3L | 151636 | Homo sapiens deltex 3-like (Drosophila) (DTX3L), |
| | | | mRNA. |
| 1980431 | DOK3 | 79930 | Homo sapiens docking protein 3 (DOK3), mRNA. |
| 5670465 | ADM | 133 | Homo sapiens adrenomedullin (ADM), mRNA. |
| 620484 | TNFRSF13B | 23495 | Homo sapiens tumor necrosis factor receptor superfamily, |
| | | | member 13B (TNFRSF13B), mRNA. |
| 1820592 | HIST2H2AA3 | 8337 | Homo sapiens histone cluster 2, H2aa3 (HIST2H2AA3), |
| | | | mRNA. |
| 610451 | HIST2H2AA3 | 8337 | Homo sapiens histone cluster 2, H2aa3 (HIST2H2AA3), |
| | | | mRNA. |
| 5220142 | LIMK2 | 3985 | Homo sapiens LIM domain kinase 2 (LIMK2), transcript |
| | | | variant 2a, mRNA. |
| 670528 | GYG1 | 2992 | Homo sapiens glycogenin 1 (GYG1), mRNA. |
| 2640619 | ZNF91 | 7644 | Homo sapiens zinc finger protein 91 (ZNF91), mRNA. |
| 110397 | LOC652755 | 652755 | PREDICTED: Homo sapiens similar to Baculoviral IAP |
| | | | repeat-containing protein 1 (Neuronal apoptosis inhibitory |
| | | | protein) (LOC652755), mRNA. |
| 2140541 | LOC389816 | 389816 | Homo sapiens cytokeratin associated protein |
| | | | (LOC389816), mRNA. |
| 6520474 | SPACA4 | 171169 | Homo sapiens sperm acrosome associated 4 (SPACA4), |
| | | | mRNA. |
| 3310538 | CD36 | 948 | Homo sapiens CD36 molecule (thrombospondin receptor) |
| | | | (CD36), transcript variant 3, mRNA. |
| 3520349 | LOC653980 | 653980 | PREDICTED: Homo sapiens similar to BTB and CNC |
| | | | homology 1, basic leucine zipper transcription factor 2, |
| | | | transcript variant 1 (LOC653980), mRNA. |
| 3420523 | RHBDF2 | 79651 | Homo sapiens rhomboid 5 homolog 2 (Drosophila) |
| | | | (RHBDF2), transcript variant 2, mRNA. |
| 650750 | RHBDF2 | 79651 | Homo sapiens rhomboid 5 homolog 2 (Drosophila) |
| | | | (RHBDF2), transcript variant 2, mRNA. |
| 840369 | LBH | 81606 | Homo sapiens limb bud and heart development homolog |
| | | | (mouse) (LBH), mRNA. |
| 4200746 | BPI | 671 | Homo sapiens bactericidal/permeability-increasing protein |
| | | | (BPI), mRNA. |
| 4200754 | ST3GAL4 | 6484 | Homo sapiens ST3 beta-galactoside alpha-2,3- |
| | | | sialyltransferase 4 (ST3GAL4), mRNA. |
| 5050274 | DHRS12 | 79758 | Homo sapiens dehydrogenase/reductase (SDR family) |
| | | | member 12 (DHRS12), transcript variant 2, mRNA. |
| 6980133 | | | HUMGS0004661 Human adult (K. Okubo) Homo sapiens |
| | | | cDNA 3, mRNA sequence |
| 6590646 | FAM26F | 441168 | Homo sapiens family with sequence similarity 26, |
| | | | member F (FAM26F), mRNA. |
| 3780193 | FCRLA | 84824 | Homo sapiens Fc receptor-like A (FCRLA), mRNA. |
| 1070477 | ALDH1A1 | 216 | Homo sapiens aldehyde dehydrogenase 1 family, member |
| | | | A1 (ALDH1A1), mRNA. |
| 830615 | ZDHHC19 | 131540 | Homo sapiens zinc finger, DHHC-type containing 19 |
| | | | (ZDHHC19), mRNA. |
| 1850463 | LOC732371 | 732371 | PREDICTED: Homo sapiens similar to mixed lineage |
| | | | kinase domain-like (LOC732371), mRNA. |
| 2370639 | C6orf150 | 115004 | Homo sapiens chromosome 6 open reading frame 150 |
| | | | (C6orf150), mRNA. |
| 1410201 | CD59 | 966 | Homo sapiens CD59 molecule, complement regulatory |
| | | | protein (CD59), transcript variant 2, mRNA. |
| 4900239 | CD274 | 29126 | Homo sapiens CD274 molecule (CD274), mRNA. |
| 940274 | GK | 2710 | Homo sapiens glycerol kinase (GK), transcript variant 2, |
| | | | mRNA. |
| 7610438 | MME | 4311 | Homo sapiens membrane metallo-endopeptidase (MME), |
| | | | transcript variant 1bis, mRNA. |
| 3940189 | JAK2 | 3717 | Homo sapiens Janus kinase 2 (a protein tyrosine kinase) |
| | | | (JAK2), mRNA. |
| 7650114 | PSCDBP | 9595 | Homo sapiens pleckstrin homology, Sec7 and coiled-coil |
| | | | domains, binding protein (PSCDBP), mRNA. |
| 6650242 | IFITM3 | 10410 | Homo sapiens interferon induced transmembrane protein 3 |
| | | | (1-8U) (IFITM3), mRNA. |
| 1820750 | STAT1 | 6772 | Homo sapiens signal transducer and activator of |
| | | | transcription 1, 91 kDa (STAT1), transcript variant alpha, |
| | | | mRNA. |
| 4810187 | STAT1 | 6772 | Homo sapiens signal transducer and activator of |
| | | | transcription 1, 91 kDa (STAT1), transcript variant alpha, |
| | | | mRNA. |
| 3440433 | | | BX092531 NCI_CGAP_Kid5 Homo sapiens cDNA clone |
| | | | IMAGp998I114659; IMAGE: 1900882, mRNA sequence |
| 1260451 | VWA5A | 4013 | Homo sapiens von Willebrand factor A domain containing |
| | | | 5A (VWA5A), transcript variant 2, mRNA. |
| 840717 | CTSL1 | 1514 | Homo sapiens cathepsin L1 (CTSL1), transcript variant 1, |
| | | | mRNA. |
| 4150189 | CTSL1 | 1514 | Homo sapiens cathepsin L1 (CTSL1), transcript variant 1, |
| | | | mRNA. |
| 1260066 | | | Homo sapiens cDNA: FLJ21199 fis, clone COL00235 |
| 6250615 | PGLYRP1 | 8993 | Homo sapiens peptidoglycan recognition protein 1 |
| | | | (PGLYRP1), mRNA. |
| 5870608 | PPP1R3B | 79660 | Homo sapiens protein phosphatase 1, regulatory |
| | | | (inhibitor) subunit 3B (PPP1R3B), mRNA. |
| 3870338 | IFI44L | 10964 | Homo sapiens interferon-induced protein 44-like (IFI44L), |
| | | | mRNA. |
| 1090132 | PARP10 | 84875 | PREDICTED: Homo sapiens poly (ADP-ribose) |
| | | | polymerase family, member 10 (PARP10), mRNA. |
| 3930601 | PARP10 | 84875 | PREDICTED: Homo sapiens poly (ADP-ribose) |
| | | | polymerase family, member 10 (PARP10), mRNA. |
| 160672 | TCF7 | 6932 | Homo sapiens transcription factor 7 (T-cell specific, |
| | | | HMG-box) (TCF7), transcript variant 2, mRNA. |
| 3180010 | SDK2 | 54549 | Homo sapiens sidekick homolog 2 (chicken) (SDK2), |
| | | | mRNA. |
| 6660162 | LRG1 | 116844 | Homo sapiens leucine-rich alpha-2-glycoprotein 1 |
| | | | (LRG1), mRNA. |
| 870561 | | | UI-H-BI3-akz-d-01-0-UI.s1 NCI_CGAP_Sub5 Homo |
| | | | sapiens cDNA clone IMAGE: 2736025 3, mRNA |
| | | | sequence |
| 6040673 | REPS2 | 9185 | Homo sapiens RALBP1 associated Eps domain containing |
| | | | 2 (REPS2), transcript variant 1, mRNA. |
| 540156 | PVRL3 | 25945 | Homo sapiens poliovirus receptor-related 3 (PVRL3), |
| | | | mRNA. |
| 2940379 | | | BX417275 Homo sapiens PLACENTA Homo sapiens |
| | | | cDNA clone CS0DE007YI01 5-PRIME, mRNA sequence |
| 990349 | MCTP2 | 55784 | Homo sapiens multiple C2 domains, transmembrane 2 |
| | | | (MCTP2), mRNA. |
| 7400372 | WSB1 | 26118 | Homo sapiens WD repeat and SOCS box-containing 1 |
| | | | (WSB1), transcript variant 3, mRNA. |
| 540433 | LOC652838 | 652838 | PREDICTED: Homo sapiens similar to lethal giant larvae |
| | | | homolog 2 isoform a (LOC652838), mRNA. |
| 1240450 | CD27 | 939 | Homo sapiens CD27 molecule (CD27), mRNA. |
| 6100022 | HIST2H2AC | 8338 | Homo sapiens histone cluster 2, H2ac (HIST2H2AC), |
| | | | mRNA. |
| 5270520 | FAIM3 | 9214 | Homo sapiens Fas apoptotic inhibitory molecule 3 |
| | | | (FAIM3), mRNA. |
| 7510537 | SCO2 | 9997 | Homo sapiens SCO cytochrome oxidase deficient |
| | | | homolog 2 (yeast) (SCO2), nuclear gene encoding |
| | | | mitochondrial protein, mRNA. |
| 1410192 | TDRD9 | 122402 | Homo sapiens tudor domain containing 9 (TDRD9), |
| | | | mRNA. |
| 5390504 | | | AGENCOURT_10185031 NIH_MGC_107 Homo sapiens |
| | | | cDNA clone IMAGE: 6569320 5, mRNA sequence |
| 3930128 | | | BX110640 Soares_testis_NHT Homo sapiens cDNA |
| | | | clone IMAGp998B094156, mRNA sequence |
| 7040176 | C20orf24 | 55969 | Homo sapiens chromosome 20 open reading frame 24 |
| | | | (C20orf24), transcript variant 1, mRNA. |
| 3940438 | NCF1 | 653361 | Homo sapiens neutrophil cytosolic factor 1 (NCF1), |
| | | | mRNA. |
| 6520180 | IL18RAP | 8807 | Homo sapiens interleukin 18 receptor accessory protein |
| | | | (IL18RAP), mRNA. |
| 130128 | LOC285016 | 285016 | Homo sapiens hypothetical protein LOC285016 |
| | | | (LOC285016), mRNA. |
| 2000110 | BRSK1 | 84446 | Homo sapiens BR serine/threonine kinase 1 (BRSK1), |
| | | | mRNA. |
| 6760593 | OSBPL10 | 114884 | Homo sapiens oxysterol binding protein-like 10 |
| | | | (OSBPL10), mRNA. |
| 3140487 | BMX | 660 | Homo sapiens BMX non-receptor tyrosine kinase (BMX), |
| | | | mRNA. |
| 3990224 | TNFRSF25 | 8718 | Homo sapiens tumor necrosis factor receptor superfamily, |
| | | | member 25 (TNFRSF25), transcript variant 10, mRNA. |
| 7510132 | OLR1 | 4973 | Homo sapiens oxidized low density lipoprotein (lectin- |
| | | | like) receptor 1 (OLR1), mRNA. |
| 5720482 | HERC5 | 51191 | Homo sapiens hect domain and RLD 5 (HERC5), mRNA. |
| 2570079 | STAT1 | 6772 | Homo sapiens signal transducer and activator of |
| | | | transcription 1, 91 kDa (STAT1), transcript variant beta, |
| | | | mRNA. |
| 4590022 | | | BX441478 Homo sapiens FETAL BRAIN Homo sapiens |
| | | | cDNA clone CS0DF018YO13 5-PRIME, mRNA |
| | | | sequence |
| 6980541 | TIMP2 | 7077 | Homo sapiens TIMP metallopeptidase inhibitor 2 |
| | | | (TIMP2), mRNA. |
| 520086 | FCGR1A | 2209 | Homo sapiens Fc fragment of IgG, high affinity Ia, |
| | | | receptor (CD64) (FCGR1A), mRNA. |
| 2570300 | IFI44 | 10561 | Homo sapiens interferon-induced protein 44 (IFI44), |
| | | | mRNA. |
| 3190537 | POLR2J3 | 548644 | Homo sapiens polymerase (RNA) II (DNA directed) |
| | | | polypeptide J3 (POLR2J3), mRNA. |
| 3890592 | | | RST5526 Athersys RAGE Library Homo sapiens cDNA, |
| | | | mRNA sequence |
| 6280543 | OASL | 8638 | Homo sapiens 2′-5′-oligoadenylate synthetase-like |
| | | | (OASL), transcript variant 1, mRNA. |
| 630619 | HPSE | 10855 | Homo sapiens heparanase (HPSE), mRNA. |
| 6620689 | MTHFD2 | 10797 | Homo sapiens methylenetetrahydrofolate dehydrogenase |
| | | | (NADP+ dependent) 2, methenyltetrahydrofolate |
| | | | cyclohydrolase (MTHFD2), nuclear gene encoding |
| | | | mitochondrial protein, transcript variant 2, mRNA. |
| 6550279 | GSTM2 | 2946 | Homo sapiens glutathione S-transferase M2 (muscle) |
| | | | (GSTM2), mRNA. |
| 6370470 | APOL1 | 8542 | Homo sapiens apolipoprotein L, 1 (APOL1), transcript |
| | | | variant 2, mRNA. |
| 5560471 | FCAR | 2204 | Homo sapiens Fc fragment of IgA, receptor for (FCAR), |
| | | | transcript variant 10, mRNA. |
| 3310121 | ATP8B2 | 57198 | Homo sapiens ATPase, class I, type 8B, member 2 |
| | | | (ATP8B2), transcript variant 1, mRNA. |
| 5270725 | COP1 | 114769 | Homo sapiens caspase-1 dominant-negative inhibitor |
| | | | pseudo-ICE (COP1), transcript variant 2, mRNA. |
| 2030170 | COP1 | 114769 | Homo sapiens caspase-1 dominant-negative inhibitor |
| | | | pseudo-ICE (COP1), transcript variant 2, mRNA. |
| 6840390 | FBXL16 | 146330 | Homo sapiens F-box and leucine-rich repeat protein 16 |
| | | | (FBXL16), mRNA. |
| 4890292 | TSHZ2 | 128553 | Homo sapiens teashirt zinc finger homeobox 2 (TSHZ2), |
| | | | mRNA. |
| 2450707 | SIGLEC16 | 400709 | Homo sapiens sialic acid binding Ig-like lectin 16 |
| | | | (gene/pseudogene) (SIGLEC16), non-coding RNA. |
| 5570039 | LOC728744 | 728744 | PREDICTED: Homo sapiens hypothetical LOC728744 |
| | | | (LOC728744), mRNA. |
| 5390204 | SCARF1 | 8578 | Homo sapiens scavenger receptor class F, member 1 |
| | | | (SCARF1), transcript variant 3, mRNA. |
| 2140059 | C1orf165 | 79656 | Homo sapiens chromosome 1 open reading frame 165 |
| | | | (C1orf165), mRNA. |
| 7040181 | PFKFB2 | 5208 | Homo sapiens 6-phosphofructo-2-kinase/fructose-2,6- |
| | | | biphosphatase 2 (PFKFB2), transcript variant 2, mRNA. |
| 5900543 | Sep-04 | 5414 | Homo sapiens septin 4 (SEPT4), transcript variant 3, |
| | | | mRNA. |
| 3710068 | WARS | 7453 | Homo sapiens tryptophanyl-tRNA synthetase (WARS), |
| | | | transcript variant 2, mRNA. |
| 3180528 | MMP9 | 4318 | Homo sapiens matrix metallopeptidase 9 (gelatinase B, |
| | | | 92 kDa gelatinase, 92 kDa type IV collagenase) (MMP9), |
| | | | mRNA. |
| 2650156 | TAP2 | 6891 | Homo sapiens transporter 2, ATP-binding cassette, sub- |
| | | | family B (MDR/TAP) (TAP2), transcript variant 2, |
| | | | mRNA. |
| 4280482 | DRAM | 55332 | Homo sapiens damage-regulated autophagy modulator |
| | | | (DRAM), mRNA. |
| 460463 | SMARCD3 | 6604 | Homo sapiens SWI/SNF related, matrix associated, actin |
| | | | dependent regulator of chromatin, subfamily d, member 3 |
| | | | (SMARCD3), transcript variant 1, mRNA. |
| 6200161 | CECR6 | 27439 | Homo sapiens cat eye syndrome chromosome region, |
| | | | candidate 6 (CECR6), mRNA. |
| 3840288 | LRFN3 | 79414 | Homo sapiens leucine rich repeat and fibronectin type III |
| | | | domain containing 3 (LRFN3), mRNA. |
| 4860753 | ASGR2 | 433 | Homo sapiens asialoglycoprotein receptor 2 (ASGR2), |
| | | | transcript variant 3, mRNA. |
| 3800576 | ASGR2 | 433 | Homo sapiens asialoglycoprotein receptor 2 (ASGR2), |
| | | | transcript variant 3, mRNA. |
| 2600747 | IFIT2 | 3433 | Homo sapiens interferon-induced protein with |
| | | | tetratricopeptide repeats 2 (IFIT2), mRNA. |
| 4290730 | LGALS3BP | 3959 | Homo sapiens lectin, galactoside-binding, soluble, 3 |
| | | | binding protein (LGALS3BP), mRNA. |
| 1980524 | GBP4 | 115361 | Homo sapiens guanylate binding protein 4 (GBP4), |
| | | | mRNA. |
| 6480403 | TXNDC3 | 51314 | Homo sapiens thioredoxin domain containing 3 |
| | | | (spermatozoa) (TXNDC3), mRNA. |
| 4570441 | IFIH1 | 64135 | Homo sapiens interferon induced with helicase C domain |
| | | | 1 (IFIH1), mRNA. |
| 5810689 | PRRG4 | 79056 | Homo sapiens proline rich Gla (G-carboxyglutamic acid) |
| | | | 4 (transmembrane) (PRRG4), mRNA. |
| 5090156 | NBL1 | 4681 | Homo sapiens neuroblastoma, suppression of |
| | | | tumorigenicity 1 (NBL1), transcript variant 1, mRNA. |
| 6980546 | CACNA1I | 8911 | Homo sapiens calcium channel, voltage-dependent, T |
| | | | type, alpha 1I subunit (CACNA1I), transcript variant 2, |
| | | | mRNA. |
| 110347 | PDK3 | 5165 | Homo sapiens pyruvate dehydrogenase kinase, isozyme 3 |
| | | | (PDK3), mRNA. |
| 290437 | HRK | 8739 | Homo sapiens harakiri, BCL2 interacting protein (contains |
| | | | only BH3 domain) (HRK), mRNA. |
| 4920110 | GADD45B | 4616 | Homo sapiens growth arrest and DNA-damage-inducible, |
| | | | beta (GADD45B), mRNA. |
| 6510754 | ALDH1A1 | 216 | Homo sapiens aldehyde dehydrogenase 1 family, member |
| | | | A1 (ALDH1A1), mRNA. |
| 5670100 | TCN2 | 6948 | Homo sapiens transcobalamin II; macrocytic anemia |
| | | | (TCN2), mRNA. |
| 2000148 | IFIT1 | 3434 | Homo sapiens interferon-induced protein with |
| | | | tetratricopeptide repeats 1 (IFIT1), transcript variant 2, |
| | | | mRNA. |
| 270240 | SLC26A8 | 116369 | Homo sapiens solute carrier family 26, member 8 |
| | | | (SLC26A8), transcript variant 1, mRNA. |
| 1260445 | SLC26A8 | 116369 | Homo sapiens solute carrier family 26, member 8 |
| | | | (SLC26A8), transcript variant 1, mRNA. |
| 7610053 | DDX60 | 55601 | Homo sapiens DEAD (Asp-Glu-Ala-Asp) box polypeptide |
| | | | 60 (DDX60), mRNA. |
| 4860746 | LOC642684 | 642684 | PREDICTED: Homo sapiens similar to Maltase- |
| | | | glucoamylase, intestinal (LOC642684), mRNA. |
| 2810673 | HOXA9 | 3205 | Homo sapiens homeobox A9 (HOXA9), mRNA. |
| 520441 | LOC642606 | 642606 | PREDICTED: Homo sapiens hypothetical protein |
| | | | LOC642606 (LOC642606), mRNA. |
| 5960747 | TRIM22 | 10346 | Homo sapiens tripartite motif-containing 22 (TRIM22), |
| | | | mRNA. |
| 3460014 | C4orf18 | 51313 | Homo sapiens chromosome 4 open reading frame 18 |
| | | | (C4orf18), transcript variant 2, mRNA. |
| 2060411 | LOC652616 | 652616 | PREDICTED: Homo sapiens similar to neutrophil |
| | | | cytosolic factor 1 (LOC652616), mRNA. |
| 4860224 | WARS | 7453 | Homo sapiens tryptophanyl-tRNA synthetase (WARS), |
| | | | transcript variant 1, mRNA. |
| 1940162 | GBP2 | 2634 | Homo sapiens guanylate binding protein 2, interferon- |
| | | | inducible (GBP2), mRNA. |
| 3290072 | | | BX116726 NCI_CGAP_Pr28 Homo sapiens cDNA clone |
| | | | IMAGp998J065569, mRNA sequence |
| 1770243 | IGF2BP3 | 10643 | Homo sapiens insulin-like growth factor 2 mRNA binding |
| | | | protein 3 (IGF2BP3), mRNA. |
| 7650097 | OASL | 8638 | Homo sapiens 2′-5′-oligoadenylate synthetase-like |
| | | | (OASL), transcript variant 2, mRNA. |
| 4220187 | DYSF | 8291 | Homo sapiens dysferlin, limb girdle muscular dystrophy |
| | | | 2B (autosomal recessive) (DYSF), mRNA. |
| 540161 | LOC552891 | 552891 | Homo sapiens hypothetical protein LOC552891 |
| | | | (LOC552891), mRNA. |
| 7650026 | MUC1 | 4582 | Homo sapiens mucin 1, cell surface associated (MUC1), |
| | | | transcript variant 6, mRNA. |
| 2340441 | SLC22A18AS | 5003 | Homo sapiens solute carrier family 22 (organic cation |
| | | | transporter), member 18 antisense (SLC22A18AS), |
| | | | mRNA. |
| 5550397 | APOL6 | 80830 | Homo sapiens apolipoprotein L, 6 (APOL6), mRNA. |
| 520408 | IFIT3 | 3437 | Homo sapiens interferon-induced protein with |
| | | | tetratricopeptide repeats 3 (IFIT3), mRNA. |
| 4120367 | LPCAT2 | 54947 | Homo sapiens lysophosphatidylcholine acyltransferase 2 |
| | | | (LPCAT2), mRNA. |
| 430019 | LOC391045 | 391045 | PREDICTED: Homo sapiens similar to Solute carrier |
| | | | family 2, facilitated glucose transporter member 3 |
| | | | (Glucose transporter type 3, brain) (LOC391045), mRNA. |
| 7100035 | IL28RA | 163702 | Homo sapiens interleukin 28 receptor, alpha (interferon, |
| | | | lambda receptor) (IL28RA), transcript variant 2, mRNA. |
| 7160592 | ACOT9 | 23597 | Homo sapiens acyl-CoA thioesterase 9 (ACOT9), |
| | | | transcript variant 2, mRNA. |
| 1410221 | S100A12 | 6283 | Homo sapiens S100 calcium binding protein A12 |
| | | | (S100A12), mRNA. |
| 6270553 | CXCL10 | 3627 | Homo sapiens chemokine (C—X—C motif) ligand 10 |
| | | | (CXCL10), mRNA. |
| 5890095 | LILRA5 | 353514 | Homo sapiens leukocyte immunoglobulin-like receptor, |
| | | | subfamily A (with TM domain), member 5 (LILRA5), |
| | | | transcript variant 1, mRNA. |
| 2350504 | ECGF1 | 1890 | Homo sapiens endothelial cell growth factor 1 (platelet- |
| | | | derived) (ECGF1), mRNA. |
| 7650128 | ECGF1 | 1890 | Homo sapiens endothelial cell growth factor 1 (platelet- |
| | | | derived) (ECGF1), mRNA. |
| 5310451 | BCL6 | 604 | Homo sapiens B-cell CLL/lymphoma 6 (zinc finger |
| | | | protein 51) (BCL6), transcript variant 2, mRNA. |
| 4150369 | GK | 2710 | Homo sapiens glycerol kinase (GK), transcript variant 1, |
| | | | mRNA. |
| 6900309 | ARSB | 411 | Homo sapiens arylsulfatase B (ARSB), transcript variant |
| | | | 1, mRNA. |
| 2230204 | OAS2 | 4939 | Homo sapiens 2′-5′-oligoadenylate synthetase 2, 69/71 kDa |
| | | | (OAS2), transcript variant 3, mRNA. |
| 4250136 | LTB4R | 1241 | Homo sapiens leukotriene B4 receptor (LTB4R), mRNA. |
| 6350634 | DYNLT1 | 6993 | Homo sapiens dynein, light chain, Tctex-type 1 |
| | | | (DYNLT1), mRNA. |
| 3940703 | RORA | 6095 | Homo sapiens RAR-related orphan receptor A (RORA), |
| | | | transcript variant 3, mRNA. |
| 2260725 | MGAM | 8972 | Homo sapiens maltase-glucoamylase (alpha-glucosidase) |
| | | | (MGAM), mRNA. |
| 2600369 | KRT72 | 140807 | Homo sapiens keratin 72 (KRT72), mRNA. |
| 4670458 | Sep-04 | 5414 | Homo sapiens septin 4 (SEPT4), transcript variant 2, |
| | | | mRNA. |
| 3420639 | Sep-04 | 5414 | Homo sapiens septin 4 (SEPT4), transcript variant 2, |
| | | | mRNA. |
| 240594 | ANXA3 | 306 | Homo sapiens annexin A3 (ANXA3), mRNA. |
| 6270075 | PITPNC1 | 26207 | Homo sapiens phosphatidylinositol transfer protein, |
| | | | cytoplasmic 1 (PITPNC1), transcript variant 1, mRNA. |
| 4920228 | FFAR2 | 2867 | Homo sapiens free fatty acid receptor 2 (FFAR2), mRNA. |
| 10136 | PML | 5371 | PREDICTED: Homo sapiens promyelocytic leukemia, |
| | | | transcript variant 12 (PML), mRNA. |
| 4890156 | RCAN3 | 11123 | Homo sapiens RCAN family member 3 (RCAN3), |
| | | | mRNA. |
| 4780075 | CEACAM8 | 1088 | Homo sapiens carcinoembryonic antigen-related cell |
| | | | adhesion molecule 8 (CEACAM8), mRNA. |
| 3850678 | LRRC25 | 126364 | Homo sapiens leucine rich repeat containing 25 |
| | | | (LRRC25), mRNA. |
| 3940504 | CD79A | 973 | Homo sapiens CD79a molecule, immunoglobulin- |
| | | | associated alpha (CD79A), transcript variant 2, mRNA. |
| 360224 | C9orf84 | 158401 | Homo sapiens chromosome 9 open reading frame 84 |
| | | | (C9orf84), transcript variant 1, mRNA. |
| 1450427 | RTP4 | 64108 | Homo sapiens receptor (chemosensory) transporter protein |
| | | | 4 (RTP4), mRNA. |
| 3610706 | BMX | 660 | Homo sapiens BMX non-receptor tyrosine kinase (BMX), |
| | | | mRNA. |
| 6940605 | | | Homo sapiens cDNA clone IMAGE: 5277162 |
| 1240424 | ANP32A | 8125 | Homo sapiens acidic (leucine-rich) nuclear |
| | | | phosphoprotein 32 family, member A (ANP32A), mRNA. |
| 7560593 | OSM | 5008 | Homo sapiens oncostatin M (OSM), mRNA. |
| 3780370 | C9orf72 | 203228 | Homo sapiens chromosome 9 open reading frame 72 |
| | | | (C9orf72), transcript variant 2, mRNA. |
| 2680523 | SAMD4A | 23034 | Homo sapiens sterile alpha motif domain containing 4A |
| | | | (SAMD4A), mRNA. |
| 5270300 | DIP2A | 23181 | Homo sapiens DIP2 disco-interacting protein 2 homolog |
| | | | A (Drosophila) (DIP2A), transcript variant 1, mRNA. |
| 7160255 | TMEM207 | 131920 | Homo sapiens transmembrane protein 207 (TMEM207), |
| | | | mRNA. |
| 5090743 | | | full-length cDNA clone CS0DI053YO09 of Placenta Cot |
| | | | 25-normalized of Homo sapiens (human) |
| 3170241 | LOC651524 | 651524 | PREDICTED: Homo sapiens similar to integrin alpha X |
| | | | precursor (LOC651524), mRNA. |
| 2230097 | TNFRSF25 | 8718 | Homo sapiens tumor necrosis factor receptor superfamily, |
| | | | member 25 (TNFRSF25), transcript variant 12, mRNA. |
| 6840767 | FRMD3 | 257019 | Homo sapiens FERM domain containing 3 (FRMD3), |
| | | | mRNA. |
| 6100379 | LOC648984 | 648984 | PREDICTED: Homo sapiens similar to Baculoviral IAP |
| | | | repeat-containing protein 1 (Neuronal apoptosis inhibitory |
| | | | protein) (LOC648984), mRNA. |
| 6220671 | PLAUR | 5329 | Homo sapiens plasminogen activator, urokinase receptor |
| | | | (PLAUR), transcript variant 2, mRNA. |
| 360475 | PLAUR | 5329 | Homo sapiens plasminogen activator, urokinase receptor |
| | | | (PLAUR), transcript variant 2, mRNA. |
| 2970019 | HIST1H4H | 8365 | Homo sapiens histone cluster 1, H4h (HIST1H4H), |
| | | | mRNA. |
| 3780047 | GBP6 | 163351 | Homo sapiens guanylate binding protein family, member |
| | | | 6 (GBP6), mRNA. |
| 7380400 | GBP6 | 163351 | Homo sapiens guanylate binding protein family, member |
| | | | 6 (GBP6), mRNA. |
| 5910064 | TRIM5 | 85363 | Homo sapiens tripartite motif-containing 5 (TRIM5), |
| | | | transcript variant delta, mRNA. |
| 4230102 | SOCS3 | 9021 | Homo sapiens suppressor of cytokine signaling 3 |
| | | | (SOCS3), mRNA. |
| 3060524 | LOC153561 | 153561 | Homo sapiens hypothetical protein LOC153561 |
| | | | (LOC153561), mRNA. |
| 4290491 | PSME1 | 5720 | Homo sapiens proteasome (prosome, macropain) activator |
| | | | subunit 1 (PA28 alpha) (PSME1), transcript variant 2, |
| | | | mRNA. |
| 3940392 | KIAA0367 | 23273 | Homo sapiens KIAA0367 (KIAA0367), mRNA. |
| 3610327 | OAS2 | 4939 | Homo sapiens 2′-5′-oligoadenylate synthetase 2, 69/71 kDa |
| | | | (OAS2), transcript variant 1, mRNA. |
| 1430360 | ABHD14A | 25864 | Homo sapiens abhydrolase domain containing 14A |
| | | | (ABHD14A), mRNA. |
| 2650605 | C4orf18 | 51313 | Homo sapiens chromosome 4 open reading frame 18 |
| | | | (C4orf18), transcript variant 2, mRNA. |
| 7400102 | ASPHD2 | 57168 | Homo sapiens aspartate beta-hydroxylase domain |
| | | | containing 2 (ASPHD2), mRNA. |
| 7160468 | DHRS9 | 10170 | Homo sapiens dehydrogenase/reductase (SDR family) |
| | | | member 9 (DHRS9), transcript variant 1, mRNA. |
| 4490079 | DHRS9 | 10170 | Homo sapiens dehydrogenase/reductase (SDR family) |
| | | | member 9 (DHRS9), transcript variant 1, mRNA. |
| 1940021 | GRN | 2896 | Homo sapiens granulin (GRN), mRNA. |
| 3710397 | EFNA1 | 1942 | Homo sapiens ephrin-A1 (EFNA1), transcript variant 1, |
| | | | mRNA. |
| 2140242 | TNFAIP6 | 7130 | Homo sapiens tumor necrosis factor, alpha-induced |
| | | | protein 6 (TNFAIP6), mRNA. |
| 4220327 | MRPL38 | 64978 | Homo sapiens mitochondrial ribosomal protein L38 |
| | | | (MRPL38), nuclear gene encoding mitochondrial protein, |
| | | | mRNA. |
| 6380040 | COL9A2 | 1298 | Homo sapiens collagen, type IX, alpha 2 (COL9A2), |
| | | | mRNA. |

TABLE 8
List of the 664 genes

| ProbeID | Symbol | Entrez_Gene_ID | Definition |
|---|---|---|---|
| 4730168 | SARS2 | 54938 | Homo sapiens seryl-tRNA synthetase 2, mitochondrial |
| | | | (SARS2), nuclear gene encoding mitochondrial protein, |
| | | | mRNA. |
| 2900224 | ENTPD4 | 9583 | Homo sapiens ectonucleoside triphosphate |
| | | | diphosphohydrolase 4 (ENTPD4), mRNA. |
| 5360398 | ERI1 | 90459 | Homo sapiens exoribonuclease 1 (ERI1), mRNA. |
| 7560114 | KLHDC8B | 200942 | Homo sapiens ketch domain containing 8B (KLHDC8B), |
| | | | mRNA. |
| 160612 | MGLL | 11343 | Homo sapiens monoglyceride lipase (MGLL), transcript |
| | | | variant 1, mRNA. |
| 2190349 | KCNJ15 | 3772 | Homo sapiens potassium inwardly-rectifying channel, |
| | | | subfamily J, member 15 (KCNJ15), transcript variant 3, |
| | | | mRNA. |
| 1500309 | | | Homo sapiens cDNA: FLJ22140 fis, clone HEP20977 |
| 5860669 | LOC100170939 | 100170939 | Homo sapiens glucuronidase, beta pseudogene |
| | | | (LOC100170939), non-coding RNA. |
| 5700725 | EPSTI1 | 94240 | Homo sapiens epithelial stromal interaction 1 (breast) |
| | | | (EPSTI1), transcript variant 2, mRNA. |
| 3450114 | FAM69B | 138311 | PREDICTED: Homo sapiens family with sequence |
| | | | similarity 69, member B (FAM69B), mRNA. |
| 6100356 | ALPL | 249 | Homo sapiens alkaline phosphatase, liver/bone/kidney |
| | | | (ALPL), transcript variant 1, mRNA. |
| 5220538 | WDFY3 | 23001 | Homo sapiens WD repeat and FYVE domain containing 3 |
| | | | (WDFY3), transcript variant 2, mRNA. |
| 1340039 | TFPI | 7035 | Homo sapiens tissue factor pathway inhibitor (lipoprotein- |
| | | | associated coagulation inhibitor) (TFPI), transcript variant |
| | | | 1, mRNA. |
| 4850731 | TFPI | 7035 | Homo sapiens tissue factor pathway inhibitor (lipoprotein- |
| | | | associated coagulation inhibitor) (TFPI), transcript variant |
| | | | 1, mRNA. |
| 2070646 | GPR84 | 53831 | Homo sapiens G protein-coupled receptor 84 (GPR84), |
| | | | mRNA. |
| 6650176 | TRMT11 | 60487 | Homo sapiens tRNA methyltransferase 11 homolog (S. cerevisiae) |
| | | | (TRMT11), mRNA. |
| 520162 | HPSE | 10855 | Homo sapiens heparanase (HPSE), mRNA. |
| 3850440 | FCER1G | 2207 | Homo sapiens Fc fragment of IgE, high affinity I, receptor |
| | | | for; gamma polypeptide (FCER1G), mRNA. |
| 130181 | ANKRD22 | 118932 | Homo sapiens ankyrin repeat domain 22 (ANKRD22), |
| | | | mRNA. |
| 620136 | LILRA5 | 353514 | Homo sapiens leukocyte immunoglobulin-like receptor, |
| | | | subfamily A (with TM domain), member 5 (LILRA5), |
| | | | transcript variant 3, mRNA. |
| 4120164 | LOC650116 | 650116 | PREDICTED: Homo sapiens hypothetical protein |
| | | | LOC650116 (LOC650116), mRNA. |
| 4290368 | PSTPIP2 | 9050 | Homo sapiens proline-serine-threonine phosphatase |
| | | | interacting protein 2 (PSTPIP2), mRNA. |
| 2630195 | VAMP5 | 10791 | Homo sapiens vesicle-associated membrane protein 5 |
| | | | (myobrevin) (VAMP5), mRNA. |
| 5570398 | FCGR1C | 100132417 | Homo sapiens Fc fragment of IgG, high affinity Ic, |
| | | | receptor (CD64) (FCGR1C), mRNA. |
| 770167 | CD79B | 974 | Homo sapiens CD79b molecule, immunoglobulin- |
| | | | associated beta (CD79B), transcript variant 3, mRNA. |
| 2370228 | CCDC65 | 85478 | Homo sapiens coiled-coil domain containing 65 |
| | | | (CCDC65), mRNA. |
| 510184 | LOC646434 | 646434 | PREDICTED: Homo sapiens hypothetical LOC646434 |
| | | | (LOC646434), mRNA. |
| 6620209 | FCGR1B | 2210 | Homo sapiens Fc fragment of IgG, high affinity Ib, |
| | | | receptor (CD64) (FCGR1B), transcript variant 2, mRNA. |
| 5960156 | MOBK1B | 55233 | Homo sapiens MOB1, Mps One Binder kinase activator- |
| | | | like 1B (yeast) (MOBK1B), mRNA. |
| 7560201 | NOC3L | 64318 | Homo sapiens nucleolar complex associated 3 homolog (S. cerevisiae) |
| | | | (NOC3L), mRNA. |
| 130609 | FCGBP | 8857 | Homo sapiens Fc fragment of IgG binding protein |
| | | | (FCGBP), mRNA. |
| 2230575 | LOC642267 | 642267 | PREDICTED: Homo sapiens similar to hypothetical |
| | | | protein LOC284701, transcript variant 2 (LOC642267), |
| | | | mRNA. |
| 3990010 | | | AGENCOURT_7914287 NIH_MGC_71 Homo sapiens |
| | | | cDNA clone IMAGE: 6156595 5, mRNA sequence |
| 3800600 | MAGED1 | 9500 | Homo sapiens melanoma antigen family D, 1 (MAGED1), |
| | | | transcript variant 2, mRNA. |
| 1510364 | GBP5 | 115362 | Homo sapiens guanylate binding protein 5 (GBP5), |
| | | | mRNA. |
| 1340241 | C5 | 727 | Homo sapiens complement component 5 (C5), mRNA. |
| 670202 | SESTD1 | 91404 | Homo sapiens SEC14 and spectrin domains 1 (SESTD1), |
| | | | mRNA. |
| 6370768 | ETV7 | 51513 | Homo sapiens ets variant 7 (ETV7), mRNA. |
| 3440095 | PSG3 | 5671 | Homo sapiens pregnancy specific beta-1-glycoprotein 3 |
| | | | (PSG3), mRNA. |
| 4890382 | ILVBL | 10994 | Homo sapiens ilvB (bacterial acetolactate synthase)-like |
| | | | (ILVBL), mRNA. |
| 50445 | LOC728262 | 728262 | PREDICTED: Homo sapiens hypothetical LOC728262 |
| | | | (LOC728262), mRNA. |
| 6580041 | GNLY | 10578 | Homo sapiens granulysin (GNLY), transcript variant |
| | | | NKG5, mRNA. |
| 580347 | LOC388572 | 388572 | PREDICTED: Homo sapiens hypothetical LOC388572 |
| | | | (LOC388572), mRNA. |
| 6250064 | IRF1 | 3659 | Homo sapiens interferon regulatory factor 1 (IRF1), |
| | | | mRNA. |
| 2850437 | LOC441124 | 441124 | PREDICTED: Homo sapiens hypothetical LOC441124 |
| | | | (LOC441124), mRNA. |
| 4280632 | GAS6 | 2621 | Homo sapiens growth arrest-specific 6 (GAS6), mRNA. |
| 1850220 | DAPP1 | 27071 | Homo sapiens dual adaptor of phosphotyrosine and 3- |
| | | | phosphoinositides (DAPP1), mRNA. |
| 3800753 | CDCA7L | 55536 | Homo sapiens cell division cycle associated 7-like |
| | | | (CDCA7L), mRNA. |
| 1470091 | IL15 | 3600 | Homo sapiens interleukin 15 (IL15), transcript variant 1, |
| | | | mRNA. |
| 2490315 | | | Homo sapiens cDNA: FLJ23098 fis, clone LNG07440 |
| 1190367 | IER3 | 8870 | Homo sapiens immediate early response 3 (IER3), mRNA. |
| 110639 | MAPK14 | 1432 | Homo sapiens mitogen-activated protein kinase 14 |
| | | | (MAPK14), transcript variant 2, mRNA. |
| 3450349 | CSF2RB | 1439 | Homo sapiens colony stimulating factor 2 receptor, beta, |
| | | | low-affinity (granulocyte-macrophage) (CSF2RB), |
| | | | mRNA. |
| 3180220 | GPR109B | 8843 | Homo sapiens G protein-coupled receptor 109B |
| | | | (GPR109B), mRNA. |
| 4610471 | HIST2H4B | 554313 | Homo sapiens histone cluster 2, H4b (HIST2H4B), |
| | | | mRNA. |
| 5890300 | DNAJC24 | 120526 | Homo sapiens DnaJ (Hsp40) homolog, subfamily C, |
| | | | member 24 (DNAJC24), mRNA. |
| 2970201 | ABHD2 | 11057 | Homo sapiens abhydrolase domain containing 2 (ABHD2), |
| | | | transcript variant 2, mRNA. |
| 6560156 | DUSP3 | 1845 | Homo sapiens dual specificity phosphatase 3 (vaccinia |
| | | | virus phosphatase VH1-related) (DUSP3), mRNA. |
| 270050 | ECE1 | 1889 | Homo sapiens endothelin converting enzyme 1 (ECE1), |
| | | | mRNA. |
| 7570324 | ID3 | 3399 | Homo sapiens inhibitor of DNA binding 3, dominant |
| | | | negative helix-loop-helix protein (ID3), mRNA. |
| 4200669 | CR1 | 1378 | Homo sapiens complement component (3b/4b) receptor 1 |
| | | | (Knops blood group) (CR1), transcript variant S, mRNA. |
| 3940356 | CR1 | 1378 | Homo sapiens complement component (3b/4b) receptor 1 |
| | | | (Knops blood group) (CR1), transcript variant S, mRNA. |
| 7560039 | TAPBP | 6892 | Homo sapiens TAP binding protein (tapasin) (TAPBP), |
| | | | transcript variant 3, mRNA. |
| 5080468 | MBOAT2 | 129642 | PREDICTED: Homo sapiens membrane bound O- |
| | | | acyltransferase domain containing 2 (MBOAT2), mRNA. |
| 2030309 | SERPING1 | 710 | Homo sapiens serpin peptidase inhibitor, clade G (C1 |
| | | | inhibitor), member 1 (SERPING1), transcript variant 2, |
| | | | mRNA. |
| 940369 | LOC100129637 | 100129637 | Homo sapiens hypothetical LOC100129637 |
| | | | (LOC100129637), non-coding RNA. XR_039552 |
| | | | XR_039796 |
| 5960731 | SOD2 | 6648 | Homo sapiens superoxide dismutase 2, mitochondrial |
| | | | (SOD2), nuclear gene encoding mitochondrial protein, |
| | | | transcript variant 1, mRNA. |
| 7570196 | TSPAN9 | 10867 | Homo sapiens tetraspanin 9 (TSPAN9), mRNA. |
| 3170273 | FER1L3 | 26509 | Homo sapiens fer-1-like 3, myoferlin (C. elegans) |
| | | | (FER1L3), transcript variant 2, mRNA. |
| 6940164 | LBH | 81606 | PREDICTED: Homo sapiens hypothetical protein |
| | | | DKFZp566J091 (LBH), mRNA. |
| 4730349 | PNKD | 25953 | Homo sapiens paroxysmal nonkinesiogenic dyskinesia |
| | | | (PNKD), transcript variant 2, mRNA. |
| 4780044 | LOC389386 | 389386 | PREDICTED: Homo sapiens misc_RNA (LOC389386), |
| | | | partial miscRNA. |
| 580343 | ZNF540 | 163255 | Homo sapiens zinc finger protein 540 (ZNF540), mRNA. |
| 5360064 | GNLY | 10578 | Homo sapiens granulysin (GNLY), transcript variant 519, |
| | | | mRNA. |
| 6650100 | WDR36 | 134430 | Homo sapiens WD repeat domain 36 (WDR36), mRNA. |
| 4810468 | SELP | 6403 | Homo sapiens selectin P (granule membrane protein |
| | | | 140 kDa, antigen CD62) (SELP), mRNA. |
| 610598 | CHI3L2 | 1117 | Homo sapiens chitinase 3-like 2 (CHI3L2), transcript |
| | | | variant 1, mRNA. |
| 5130025 | LOC100134703 | 100134703 | PREDICTED: Homo sapiens misc_RNA |
| | | | (LOC100134703), partial miscRNA. |
| 4250082 | CHD6 | 84181 | Homo sapiens chromodomain helicase DNA binding |
| | | | protein 6 (CHD6), mRNA. |
| 4120561 | LIMK2 | 3985 | Homo sapiens LIM domain kinase 2 (LIMK2), transcript |
| | | | variant 1, mRNA. |
| 7050370 | ANKRD9 | 122416 | Homo sapiens ankyrin repeat domain 9 (ANKRD9), |
| | | | mRNA. |
| 940220 | NOD2 | 64127 | Homo sapiens nucleotide-binding oligomerization domain |
| | | | containing 2 (NOD2), mRNA. |
| 3360491 | MCTP1 | 79772 | Homo sapiens multiple C2 domains, transmembrane 1 |
| | | | (MCTP1), transcript variant L, mRNA. |
| 4010139 | BANK1 | 55024 | Homo sapiens B-cell scaffold protein with ankyrin repeats |
| | | | 1 (BANK1), transcript variant 2, mRNA. |
| 2230678 | ACACB | 32 | Homo sapiens acetyl-Coenzyme A carboxylase beta |
| | | | (ACACB), mRNA. |
| 3610719 | SAMD3 | 154075 | Homo sapiens sterile alpha motif domain containing 3 |
| | | | (SAMD3), transcript variant 1, mRNA. |
| 1030167 | SAMD3 | 154075 | Homo sapiens sterile alpha motif domain containing 3 |
| | | | (SAMD3), transcript variant 1, mRNA. |
| 4670592 | MYOF | 26509 | Homo sapiens myoferlin (MYOF), transcript variant 1, |
| | | | mRNA. |
| 2650093 | NFAT5 | 10725 | Homo sapiens nuclear factor of activated T-cells 5, |
| | | | tonicity-responsive (NFAT5), transcript variant 2, mRNA. |
| 2680050 | SIRPG | 55423 | Homo sapiens signal-regulatory protein gamma (SIRPG), |
| | | | transcript variant 2, mRNA. |
| 730008 | | | xv24e03.x1 Soares_NFL_T_GBC_S1 Homo sapiens |
| | | | cDNA clone IMAGE: 2814076 3, mRNA sequence |
| 1450139 | FCGR3B | 2215 | Homo sapiens Fc fragment of IgG, low affinity IIIb, |
| | | | receptor (CD16b) (FCGR3B), mRNA. |
| 7160148 | ZNF329 | 79673 | Homo sapiens zinc finger protein 329 (ZNF329), mRNA. |
| 6550382 | CAMKK2 | 10645 | Homo sapiens calcium/calmodulin-dependent protein |
| | | | kinase kinase 2, beta (CAMKK2), transcript variant 4, |
| | | | mRNA. |
| 5420646 | CETP | 1071 | Homo sapiens cholesteryl ester transfer protein, plasma |
| | | | (CETP), mRNA. |
| 3830220 | UBASH3A | 53347 | Homo sapiens ubiquitin associated and SH3 domain |
| | | | containing, A (UBASH3A), transcript variant 2, mRNA. |
| 1470639 | LOC100129905 | 100129905 | PREDICTED: Homo sapiens similar to hCG2027326 |
| | | | (LOC100129905), mRNA. |
| 1510619 | LOC100129905 | 100129905 | PREDICTED: Homo sapiens similar to hCG2027326 |
| | | | (LOC100129905), mRNA. |
| 5560246 | TPM1 | 7168 | Homo sapiens tropomyosin 1 (alpha) (TPM1), transcript |
| | | | variant 3, mRNA. |
| 5360156 | IFITM1 | 8519 | Homo sapiens interferon induced transmembrane protein 1 |
| | | | (9-27) (IFITM1), mRNA. |
| 3830228 | GPR109A | 338442 | Homo sapiens G protein-coupled receptor 109A |
| | | | (GPR109A), mRNA. |
| 2510220 | IFI35 | 3430 | Homo sapiens interferon-induced protein 35 (IFI35), |
| | | | mRNA. |
| 1470382 | IRF7 | 3665 | Homo sapiens interferon regulatory factor 7 (IRF7), |
| | | | transcript variant b, mRNA. |
| 6400176 | IRF7 | 3665 | Homo sapiens interferon regulatory factor 7 (IRF7), |
| | | | transcript variant b, mRNA. |
| 3400392 | FPR2 | 2358 | Homo sapiens formyl peptide receptor 2 (FPR2), transcript |
| | | | variant 1, mRNA. |
| 5310445 | KREMEN1 | 83999 | Homo sapiens kringle containing transmembrane protein 1 |
| | | | (KREMEN1), transcript variant 4, mRNA. |
| 3390301 | KREMEN1 | 83999 | Homo sapiens kringle containing transmembrane protein 1 |
| | | | (KREMEN1), transcript variant 4, mRNA. |
| 5310754 | VNN1 | 8876 | Homo sapiens vanin 1 (VNN1), mRNA. |
| 6840035 | GBP1 | 2633 | Homo sapiens guanylate binding protein 1, interferon- |
| | | | inducible, 67 kDa (GBP1), mRNA. |
| 2190148 | GBP1 | 2633 | Homo sapiens guanylate binding protein 1, interferon- |
| | | | inducible, 67 kDa (GBP1), mRNA. |
| 240053 | GCH1 | 2643 | Homo sapiens GTP cyclohydrolase 1 (GCH1), transcript |
| | | | variant 4, mRNA. |
| 5220706 | FLJ33630 | 644873 | Homo sapiens hypothetical LOC644873 (FLJ33630), non- |
| | | | coding RNA. |
| 3800398 | FBXO6 | 26270 | Homo sapiens F-box protein 6 (FBXO6), mRNA. |
| 5900524 | LACTB | 114294 | Homo sapiens lactamase, beta (LACTB), nuclear gene |
| | | | encoding mitochondrial protein, transcript variant 2, |
| | | | mRNA. |
| 1570367 | IL18R1 | 8809 | Homo sapiens interleukin 18 receptor 1 (IL18R1), mRNA. |
| 840037 | | | UI-H-BI2-ahe-g-01-0-UI.s1 NCI_CGAP_Sub4 Homo |
| | | | sapiens cDNA clone IMAGE: 2726592 3, mRNA sequence |
| 780324 | ZNF337 | 26152 | Homo sapiens zinc finger protein 337 (ZNF337), mRNA. |
| 650753 | AFF3 | 3899 | Homo sapiens AF4/FMR2 family, member 3 (AFF3), |
| | | | transcript variant 2, mRNA. |
| 1430711 | KLF12 | 11278 | Homo sapiens Kruppel-like factor 12 (KLF12), mRNA. |
| 4860280 | | | Homo sapiens cDNA FLJ31407 fis, clone NT2NE2000137 |
| 520553 | NCALD | 83988 | Homo sapiens neurocalcin delta (NCALD), transcript |
| | | | variant 3, mRNA. |
| 1400373 | SLA | 6503 | Homo sapiens Src-like-adaptor (SLA), transcript variant 1, |
| | | | mRNA. |
| 3310682 | DOCK5 | 80005 | Homo sapiens dedicator of cytokinesis 5 (DOCK5), |
| | | | mRNA. |
| 1570129 | TRAFD1 | 10906 | Homo sapiens TRAF-type zinc finger domain containing 1 |
| | | | (TRAFD1), mRNA. |
| 3440377 | PCGF5 | 84333 | Homo sapiens polycomb group ring finger 5 (PCGF5), |
| | | | mRNA. |
| 6200577 | CREB5 | 9586 | Homo sapiens cAMP responsive element binding protein 5 |
| | | | (CREB5), transcript variant 1, mRNA. |
| 4730059 | BATF2 | 116071 | Homo sapiens basic leucine zipper transcription factor, |
| | | | ATF-like 2 (BATF2), mRNA. |
| 3420241 | SLC2A14 | 144195 | Homo sapiens solute carrier family 2 (facilitated glucose |
| | | | transporter), member 14 (SLC2A14), mRNA. |
| 7050519 | CLEC4D | 338339 | Homo sapiens C-type lectin domain family 4, member D |
| | | | (CLEC4D), mRNA. |
| 7510647 | CACNA1E | 777 | Homo sapiens calcium channel, voltage-dependent, R type, |
| | | | alpha 1E subunit (CACNA1E), mRNA. |
| 2570328 | TRIB2 | 28951 | Homo sapiens tribbles homolog 2 (Drosophila) (TRIB2), |
| | | | mRNA. |
| 70180 | LOC100133823 | 100133823 | PREDICTED: Homo sapiens misc_RNA |
| | | | (LOC100133823), miscRNA. |
| 4640768 | VPREB3 | 29802 | Homo sapiens pre-B lymphocyte gene 3 (VPREB3), |
| | | | mRNA. |
| 4480279 | Sep-01 | 1731 | Homo sapiens septin 1 (SEPT1), mRNA. XM_944608 |
| | | | XM_944610 |
| 2450392 | LOC642780 | 642780 | PREDICTED: Homo sapiens similar to hypothetical |
| | | | protein LOC284701, transcript variant 2 (LOC642780), |
| | | | mRNA. |
| 2630224 | LOC93622 | 93622 | PREDICTED: Homo sapiens hypothetical protein |
| | | | BC006130 (LOC93622), misc RNA. |
| 3840092 | PFAS | 5198 | Homo sapiens phosphoribosylformylglycinamidine |
| | | | synthase (FGAR amidotransferase) (PFAS), mRNA. |
| 2340327 | CD320 | 51293 | Homo sapiens CD320 molecule (CD320), mRNA. |
| 620019 | RPS26P10 | 401470 | PREDICTED: Homo sapiens ribosomal protein S26 |
| | | | pseudogene 10 (RPS26P10), mRNA. |
| 1070189 | AXIN2 | 8313 | Homo sapiens axin 2 (conductin, axil) (AXIN2), mRNA. |
| 6510735 | DDX60L | 91351 | Homo sapiens DEAD (Asp-Glu-Ala-Asp) box polypeptide |
| | | | 60-like (DDX60L), mRNA. |
| 730528 | PLAUR | 5329 | Homo sapiens plasminogen activator, urokinase receptor |
| | | | (PLAUR), transcript variant 1, mRNA. |
| 5050382 | SPARC | 6678 | Homo sapiens secreted protein, acidic, cysteine-rich |
| | | | (osteonectin) (SPARC), mRNA. |
| 4150014 | CEACAM1 | 634 | Homo sapiens carcinoembryonic antigen-related cell |
| | | | adhesion molecule 1 (biliary glycoprotein) (CEACAM1), |
| | | | transcript variant 2, mRNA. |
| 5700753 | CEACAM1 | 634 | Homo sapiens carcinoembryonic antigen-related cell |
| | | | adhesion molecule 1 (biliary glycoprotein) (CEACAM1), |
| | | | transcript variant 2, mRNA. |
| 1980768 | SIAH2 | 6478 | Homo sapiens seven in absentia homolog 2 (Drosophila) |
| | | | (SIAH2), mRNA. |
| 5960343 | PRIC285 | 85441 | Homo sapiens peroxisomal proliferator-activated receptor |
| | | | A interacting complex 285 (PRIC285), transcript variant 2, |
| | | | mRNA. |
| 6560152 | PPP2R3A | 5523 | Homo sapiens protein phosphatase 2 (formerly 2A), |
| | | | regulatory subunit B″, alpha (PPP2R3A), transcript variant |
| | | | 1, mRNA. |
| 1820725 | LRRK2 | 120892 | Homo sapiens leucine-rich repeat kinase 2 (LRRK2), |
| | | | mRNA. |
| 3520291 | LRRK2 | 120892 | Homo sapiens leucine-rich repeat kinase 2 (LRRK2), |
| | | | mRNA. |
| 4390398 | LCN2 | 3934 | Homo sapiens lipocalin 2 (LCN2), mRNA. |
| 1240097 | BAZ1A | 11177 | Homo sapiens bromodomain adjacent to zinc finger |
| | | | domain, 1A (BAZ1A), transcript variant 1, mRNA. |
| 2650113 | NR1I2 | 8856 | Homo sapiens nuclear receptor subfamily 1, group I, |
| | | | member 2 (NR1I2), transcript variant 3, mRNA. |
| 3610647 | URG4 | 55665 | Homo sapiens up-regulated gene 4 (URG4), nuclear gene |
| | | | encoding mitochondrial protein, transcript variant 2, |
| | | | mRNA. |
| 10440 | MARS2 | 92935 | Homo sapiens methionyl-tRNA synthetase 2, |
| | | | mitochondrial (MARS2), nuclear gene encoding |
| | | | mitochondrial protein, mRNA. |
| 2750575 | | | full-length cDNA clone CS0CAP005YH21 of Thymus of |
| | | | Homo sapiens (human) |
| 6200193 | TncRNA | 283131 | PREDICTED: Homo sapiens trophoblast-derived |
| | | | noncoding RNA (TncRNA), misc RNA. |
| 3460053 | SIPA1L2 | 57568 | Homo sapiens signal-induced proliferation-associated 1 |
| | | | like 2 (SIPA1L2), mRNA. |
| 6100347 | PTPRE | 5791 | Homo sapiens protein tyrosine phosphatase, receptor type, |
| | | | E (PTPRE), transcript variant 2, mRNA. |
| 6840246 | CTSA | 5476 | Homo sapiens cathepsin A (CTSA), transcript variant 1, |
| | | | mRNA. |
| 1580435 | TGM2 | 7052 | Homo sapiens transglutaminase 2 (C polypeptide, protein- |
| | | | glutamine-gamma-glutamyltransferase) (TGM2), transcript |
| | | | variant 1, mRNA. |
| 2970575 | CCL4L2 | 388372 | Homo sapiens chemokine (C-C motif) ligand 4-like 2 |
| | | | (CCL4L2), mRNA. |
| 7400487 | | | QV1-BT0630-040400-132-d11 BT0630 Homo sapiens |
| | | | cDNA, mRNA sequence |
| 1510026 | FLVCR2 | 55640 | Homo sapiens feline leukemia virus subgroup C cellular |
| | | | receptor family, member 2 (FLVCR2), mRNA. |
| 3420593 | LMNB1 | 4001 | Homo sapiens lamin B1 (LMNB1), mRNA. |
| 6660368 | MAP4K1 | 11184 | Homo sapiens mitogen-activated protein kinase kinase |
| | | | kinase kinase 1 (MAP4K1), transcript variant 1, mRNA. |
| 6450692 | PSMB9 | 5698 | Homo sapiens proteasome (prosome, macropain) subunit, |
| | | | beta type, 9 (large multifunctional peptidase 2) (PSMB9), |
| | | | transcript variant 1, mRNA. |
| 4780128 | ATF3 | 467 | Homo sapiens activating transcription factor 3 (ATF3), |
| | | | transcript variant 4, mRNA. |
| 5050347 | CD5 | 921 | Homo sapiens CD5 molecule (CD5), mRNA. |
| 5310411 | H2AFJ | 55766 | Homo sapiens H2A histone family, member J (H2AFJ), |
| | | | transcript variant 1, mRNA. |
| 6590131 | HP | 3240 | Homo sapiens haptoglobin (HP), mRNA. |
| 3420228 | PPP1R13B | 23368 | Homo sapiens protein phosphatase 1, regulatory (inhibitor) |
| | | | subunit 13B (PPP1R13B), mRNA. |
| 6110630 | HIST1H2BK | 85236 | Homo sapiens histone cluster 1, H2bk (HIST1H2BK), |
| | | | mRNA. |
| 5050402 | HIST1H2BK | 85236 | Homo sapiens histone cluster 1, H2bk (HIST1H2BK), |
| | | | mRNA. |
| 4670414 | TMEM140 | 55281 | Homo sapiens transmembrane protein 140 (TMEM140), |
| | | | mRNA. |
| 6330471 | BLK | 640 | Homo sapiens B lymphoid tyrosine kinase (BLK), mRNA. |
| 2190019 | CD160 | 11126 | Homo sapiens CD160 molecule (CD160), mRNA. |
| 1340403 | LOC100128460 | 100128460 | PREDICTED: Homo sapiens misc_RNA |
| | | | (LOC100128460), miscRNA. |
| 7210136 | CD19 | 930 | Homo sapiens CD19 molecule (CD19), mRNA. |
| 3440360 | ZNF438 | 220929 | Homo sapiens zinc finger protein 438 (ZNF438), mRNA. |
| 2810470 | C6orf25 | 80739 | Homo sapiens chromosome 6 open reading frame 25 |
| | | | (C6orf25), transcript variant 4, mRNA. |
| 4850168 | SLC22A4 | 6583 | Homo sapiens solute carrier family 22 (organic |
| | | | cation/ergothioneine transporter), member 4 (SLC22A4), |
| | | | mRNA. |
| 6480059 | ACTA2 | 59 | Homo sapiens actin, alpha 2, smooth muscle, aorta |
| | | | (ACTA2), mRNA. |
| 3780709 | KIAA1632 | 57724 | Homo sapiens KIAA1632 (KIAA1632), mRNA. |
| 6100136 | CASP4 | 837 | Homo sapiens caspase 4, apoptosis-related cysteine |
| | | | peptidase (CASP4), transcript variant gamma, mRNA. |
| 6040398 | SLC22A4 | 6583 | Homo sapiens solute carrier family 22 (organic |
| | | | cation/ergothioneine transporter), member 4 (SLC22A4), |
| | | | mRNA. |
| 5910543 | GLOD4 | 51031 | Homo sapiens glyoxalase domain containing 4 (GLOD4), |
| | | | mRNA. |
| 2710452 | POU2AF1 | 5450 | Homo sapiens POU class 2 associating factor 1 |
| | | | (POU2AF1), mRNA. |
| 6420750 | TLR2 | 7097 | Homo sapiens toll-like receptor 2 (TLR2), mRNA. |
| 6590669 | RAB24 | 53917 | Homo sapiens RAB24, member RAS oncogene family |
| | | | (RAB24), transcript variant 1, mRNA. |
| 5910300 | RAB24 | 53917 | Homo sapiens RAB24, member RAS oncogene family |
| | | | (RAB24), transcript variant 1, mRNA. |
| 2760689 | MAGEE1 | 57692 | Homo sapiens melanoma antigen family E, 1 (MAGEE1), |
| | | | mRNA. |
| 540091 | STEAP4 | 79689 | Homo sapiens STEAP family member 4 (STEAP4), |
| | | | mRNA. |
| 3420373 | SOD2 | 6648 | Homo sapiens superoxide dismutase 2, mitochondrial |
| | | | (SOD2), nuclear gene encoding mitochondrial protein, |
| | | | transcript variant 3, mRNA. |
| 6280458 | BCL6 | 604 | Homo sapiens B-cell CLL/lymphoma 6 (zinc finger |
| | | | protein 51) (BCL6), transcript variant 1, mRNA. |
| 5050086 | MPL | 4352 | Homo sapiens myeloproliferative leukemia virus oncogene |
| | | | (MPL), mRNA. |
| 1660681 | CCDC102A | 92922 | Homo sapiens coiled-coil domain containing 102A |
| | | | (CCDC102A), mRNA. |
| 1410278 | PF4V1 | 5197 | Homo sapiens platelet factor 4 variant 1 (PF4V1), mRNA. |
| 4250630 | KLRF1 | 51348 | Homo sapiens killer cell lectin-like receptor subfamily F, |
| | | | member 1 (KLRF1), mRNA. |
| 630255 | LOC652578 | 652578 | PREDICTED: Homo sapiens similar to Fc fragment of |
| | | | IgG, low affinity IIIa, receptor for (CD16) (LOC652578), |
| | | | mRNA. |
| 6900014 | PHAX | 51808 | Homo sapiens phosphorylated adaptor for RNA export |
| | | | (PHAX), mRNA. |
| 5890075 | LIN7A | 8825 | Homo sapiens lin-7 homolog A (C. elegans) (LIN7A), |
| | | | mRNA. |
| 3360431 | ZAK | 51776 | Homo sapiens sterile alpha motif and leucine zipper |
| | | | containing kinase AZK (ZAK), transcript variant 1, |
| | | | mRNA. |
| 7570184 | LOC283663 | 283663 | Homo sapiens hypothetical LOC283663 (LOC283663), |
| | | | non-coding RNA. |
| 5390328 | KCNJ15 | 3772 | Homo sapiens potassium inwardly-rectifying channel, |
| | | | subfamily J, member 15 (KCNJ15), transcript variant 1, |
| | | | mRNA. |
| 1050215 | KCNJ15 | 3772 | Homo sapiens potassium inwardly-rectifying channel, |
| | | | subfamily J, member 15 (KCNJ15), transcript variant 1, |
| | | | mRNA. |
| 4570164 | LOC389386 | 389386 | PREDICTED: Homo sapiens misc_RNA (LOC389386), |
| | | | partial miscRNA. |
| 6020097 | SERPINA1 | 5265 | Homo sapiens serpin peptidase inhibitor, clade A (alpha-1 |
| | | | antiproteinase, antitrypsin), member 1 (SERPINA1), |
| | | | transcript variant 2, mRNA. |
| 3850202 | IGSF6 | 10261 | Homo sapiens immunoglobulin superfamily, member 6 |
| | | | (IGSF6), mRNA. |
| 620403 | LOC400759 | 400759 | Homo sapiens similar to Interferon-induced guanylate- |
| | | | binding protein 1 (GTP-binding protein 1) (Guanine |
| | | | nucleotide-binding protein 1) (HuGBP-1) (LOC400759) |
| | | | on chromosome 1. |
| 6280470 | LOC728519 | 728519 | PREDICTED: Homo sapiens similar to Baculoviral IAP |
| | | | repeat-containing protein 1 (Neuronal apoptosis inhibitory |
| | | | protein) (LOC728519), mRNA. |
| 2630577 | CAV2 | 858 | Homo sapiens caveolin 2 (CAV2), transcript variant 1, |
| | | | mRNA. |
| 5570114 | GADD45G | 10912 | Homo sapiens growth arrest and DNA-damage-inducible, |
| | | | gamma (GADD45G), mRNA. |
| 1300139 | GNG7 | 2788 | Homo sapiens guanine nucleotide binding protein (G |
| | | | protein), gamma 7 (GNG7), mRNA. |
| 2710091 | FCGR2C | 9103 | Homo sapiens Fc fragment of IgG, low affinity IIc, |
| | | | receptor for (CD32) (FCGR2C), transcript variant 3, |
| | | | mRNA. |
| 6840020 | TNFSF13B | 10673 | Homo sapiens tumor necrosis factor (ligand) superfamily, |
| | | | member 13b (TNFSF13B), transcript variant 1, mRNA. |
| 5490743 | KIF1B | 23095 | Homo sapiens kinesin family member 1B (KIF1B), |
| | | | transcript variant 2, mRNA. |
| 2360719 | IRAK3 | 11213 | Homo sapiens interleukin-1 receptor-associated kinase 3 |
| | | | (IRAK3), mRNA. |
| 4880537 | LIMA1 | 51474 | Homo sapiens LIM domain and actin binding 1 (LIMA1), |
| | | | mRNA. |
| 1050020 | NLRC4 | 58484 | Homo sapiens NLR family, CARD domain containing 4 |
| | | | (NLRC4), mRNA. |
| 450753 | HIST1H2BC | 8347 | Homo sapiens histone cluster 1, H2bc (HIST1H2BC), |
| | | | mRNA. |
| 5870192 | SKAP1 | 8631 | Homo sapiens src kinase associated phosphoprotein 1 |
| | | | (SKAP1), transcript variant 2, mRNA. |
| 2070193 | LOC100128805 | 100128805 | PREDICTED: Homo sapiens hypothetical protein |
| | | | LOC100128805 (LOC100128805), mRNA. |
| 3390121 | CASP5 | 838 | Homo sapiens caspase 5, apoptosis-related cysteine |
| | | | peptidase (CASP5), mRNA. |
| 5890386 | LOC642103 | 642103 | PREDICTED: Homo sapiens similar to Maltase- |
| | | | glucoamylase, intestinal (LOC642103), mRNA. |
| 1010544 | GAR1 | 54433 | Homo sapiens GAR1 ribonucleoprotein homolog (yeast) |
| | | | (GAR1), transcript variant 1, mRNA. |
| 2060047 | B4GALT5 | 9334 | Homo sapiens UDP-Gal:betaGlcNAc beta 1,4- |
| | | | galactosyltransferase, polypeptide 5 (B4GALT5), mRNA. |
| 4760433 | C16orf7 | 9605 | Homo sapiens chromosome 16 open reading frame 7 |
| | | | (C16orf7), mRNA. |
| 2370255 | ALDH18A1 | 5832 | Homo sapiens aldehyde dehydrogenase 18 family, member |
| | | | A1 (ALDH18A1), nuclear gene encoding mitochondrial |
| | | | protein, transcript variant 1, mRNA. |
| 20441 | | | Homo sapiens cDNA FLJ20182 fis, clone COLF0190 |
| 130433 | CTTN | 2017 | Homo sapiens cortactin (CTTN), transcript variant 1, |
| | | | mRNA. |
| 2320689 | LOC653610 | 653610 | PREDICTED: Homo sapiens similar to Histone H2A.o |
| | | | (H2A/o) (H2A.2) (H2a-615) (LOC653610), mRNA. |
| 5130767 | MAPK14 | 1432 | Homo sapiens mitogen-activated protein kinase 14 |
| | | | (MAPK14), transcript variant 1, mRNA. |
| 5700615 | LOC100129552 | 100129552 | PREDICTED: Homo sapiens misc_RNA |
| | | | (LOC100129552), miscRNA. |
| 2480544 | KCNMB1 | 3779 | Homo sapiens potassium large conductance calcium- |
| | | | activated channel, subfamily M, beta member 1 |
| | | | (KCNMB1), mRNA. |
| 3370086 | P2RX7 | 5027 | Homo sapiens purinergic receptor P2X, ligand-gated ion |
| | | | channel, 7 (P2RX7), mRNA. |
| 840685 | IL1B | 3553 | Homo sapiens interleukin 1, beta (IL1B), mRNA. |
| 6450397 | CCNB1IP1 | 57820 | Homo sapiens cyclin B1 interacting protein 1 |
| | | | (CCNB1IP1), transcript variant 2, mRNA. |
| 4490403 | IQCB1 | 9657 | Homo sapiens IQ motif containing B1 (IQCB1), transcript |
| | | | variant 3, mRNA. |
| 4230678 | HIST2H2BE | 8349 | Homo sapiens histone cluster 2, H2be (HIST2H2BE), |
| | | | mRNA. |
| 1260270 | AIM2 | 9447 | Homo sapiens absent in melanoma 2 (AIM2), mRNA. |
| 6400414 | LOC650546 | 650546 | PREDICTED: Homo sapiens similar to ubiquitin specific |
| | | | protease 32, transcript variant 1 (LOC650546), mRNA. |
| 4180148 | LOC650546 | 650546 | PREDICTED: Homo sapiens similar to ubiquitin specific |
| | | | protease 32, transcript variant 1 (LOC650546), mRNA. |
| 1980184 | P2RY10 | 27334 | Homo sapiens purinergic receptor P2Y, G-protein coupled, |
| | | | 10 (P2RY10), transcript variant 2, mRNA. |
| 3450044 | LOC100128485 | 100128485 | PREDICTED: Homo sapiens similar to conserved |
| | | | hypothetical protein (LOC100128485), mRNA. |
| 4860681 | GUCY1A3 | 2982 | Homo sapiens guanylate cyclase 1, soluble, alpha 3 |
| | | | (GUCY1A3), mRNA. |
| 6760148 | CLEC2D | 29121 | Homo sapiens C-type lectin domain family 2, member D |
| | | | (CLEC2D), transcript variant 1, mRNA. |
| 290730 | HIST1H2BD | 3017 | Homo sapiens histone cluster 1, H2bd (HIST1H2BD), |
| | | | transcript variant 2, mRNA. |
| 6200669 | HIST1H2BD | 3017 | Homo sapiens histone cluster 1, H2bd (HIST1H2BD), |
| | | | transcript variant 2, mRNA. |
| 5390427 | NAIP | 4671 | Homo sapiens NLR family, apoptosis inhibitory protein |
| | | | (NAIP), transcript variant 1, mRNA. |
| 5340240 | NAIP | 4671 | Homo sapiens NLR family, apoptosis inhibitory protein |
| | | | (NAIP), transcript variant 1, mRNA. |
| 7550358 | NELL2 | 4753 | Homo sapiens NEL-like 2 (chicken) (NELL2), mRNA. |
| 5810136 | SLAMF1 | 6504 | Homo sapiens signaling lymphocytic activation molecule |
| | | | family member 1 (SLAMF1), mRNA. |
| 4860600 | MAPK14 | 1432 | Homo sapiens mitogen-activated protein kinase 14 |
| | | | (MAPK14), transcript variant 3, mRNA. |
| 6940402 | CUTL1 | 1523 | Homo sapiens cut-like 1, CCAAT displacement protein |
| | | | (Drosophila) (CUTL1), transcript variant 1, mRNA. |
| 2810064 | BTLA | 151888 | Homo sapiens B and T lymphocyte associated (BTLA), |
| | | | mRNA. |
| 1010360 | GCH1 | 2643 | Homo sapiens GTP cyclohydrolase 1 (GCH1), transcript |
| | | | variant 3, mRNA. |
| 1170653 | PDGFA | 5154 | Homo sapiens platelet-derived growth factor alpha |
| | | | polypeptide (PDGFA), transcript variant 2, mRNA. |
| 2230538 | LRRN3 | 54674 | Homo sapiens leucine rich repeat neuronal 3 (LRRN3), |
| | | | transcript variant 1, mRNA. |
| 5810598 | | | Homo sapiens cDNA clone MGC: 13035 |
| | | | IMAGE: 3613409, complete cds |
| 5270377 | LOC728790 | 728790 | PREDICTED: Homo sapiens hypothetical LOC728790 |
| | | | (LOC728790), mRNA. |
| 1510735 | NTN3 | 4917 | Homo sapiens netrin 3 (NTN3), mRNA. |
| 4880010 | ZBP1 | 81030 | Homo sapiens Z-DNA binding protein 1 (ZBP1), mRNA. |
| 4010270 | LOC440731 | 440731 | PREDICTED: Homo sapiens hypothetical LOC440731, |
| | | | transcript variant 2 (LOC440731), mRNA. |
| 60470 | STX11 | 8676 | Homo sapiens syntaxin 11 (STX11), mRNA. |
| 5910019 | C1QB | 713 | Homo sapiens complement component 1, q subcomponent, |
| | | | B chain (C1QB), mRNA. |
| 6620630 | XK | 7504 | Homo sapiens X-linked Kx blood group (McLeod |
| | | | syndrome) (XK), mRNA. |
| 6580717 | ZMYND15 | 84225 | Homo sapiens zinc finger, MYND-type containing 15 |
| | | | (ZMYND15), transcript variant 2, mRNA. |
| 5360093 | SECTM1 | 6398 | Homo sapiens secreted and transmembrane 1 (SECTM1), |
| | | | mRNA. |
| 5290647 | CXCR5 | 643 | Homo sapiens chemokine (C—X—C motif) receptor 5 |
| | | | (CXCR5), transcript variant 2, mRNA. |
| 3890400 | CXCR5 | 643 | Homo sapiens chemokine (C—X—C motif) receptor 5 |
| | | | (CXCR5), transcript variant 2, mRNA. |
| 460438 | FAS | 355 | Homo sapiens Fas (TNF receptor superfamily, member 6) |
| | | | (FAS), transcript variant 3, mRNA. |
| 2710709 | FCGR1B | 2210 | Homo sapiens Fc fragment of IgG, high affinity Ib, |
| | | | receptor (CD64) (FCGR1B), transcript variant 1, mRNA. |
| 4040037 | EBI2 | 1880 | Homo sapiens Epstein-Barr virus induced gene 2 |
| | | | (lymphocyte-specific G protein-coupled receptor) (EBI2), |
| | | | mRNA. |
| 4490520 | EBI2 | 1880 | Homo sapiens Epstein-Barr virus induced gene 2 |
| | | | (lymphocyte-specific G protein-coupled receptor) (EBI2), |
| | | | mRNA. |
| 3800050 | ADCY3 | 109 | Homo sapiens adenylate cyclase 3 (ADCY3), mRNA. |
| 6450154 | LSS | 4047 | Homo sapiens lanosterol synthase (2,3-oxidosqualene- |
| | | | lanosterol cyclase) (LSS), transcript variant 1, mRNA. |
| 830440 | TLR5 | 7100 | Homo sapiens toll-like receptor 5 (TLR5), mRNA. |
| 3060523 | NAMPT | 10135 | Homo sapiens nicotinamide phosphoribosyltransferase |
| | | | (NAMPT), mRNA. |
| 1260482 | GZMK | 3003 | Homo sapiens granzyme K (granzyme 3; tryptase II) |
| | | | (GZMK), mRNA. |
| 5560270 | CARD17 | 440068 | Homo sapiens caspase recruitment domain family, member |
| | | | 17 (CARD17), mRNA. |
| 3930368 | CARD17 | 440068 | Homo sapiens caspase recruitment domain family, member |
| | | | 17 (CARD17), mRNA. |
| 7320731 | MAP3K4 | 4216 | Homo sapiens mitogen-activated protein kinase kinase |
| | | | kinase 4 (MAP3K4), transcript variant 1, mRNA. |
| 4230735 | FAM129A | 116496 | Homo sapiens family with sequence similarity 129, |
| | | | member A (FAM129A), transcript variant 2, mRNA. |
| 7160750 | MIIP | 60672 | Homo sapiens migration and invasion inhibitory protein |
| | | | (MIIP), mRNA. |
| 6480064 | POLR1E | 64425 | Homo sapiens polymerase (RNA) I polypeptide E, 53 kDa |
| | | | (POLR1E), mRNA. |
| 4290056 | C17orf60 | 284021 | Homo sapiens chromosome 17 open reading frame 60 |
| | | | (C17orf60), mRNA. |
| 3190053 | LOC642816 | 642816 | PREDICTED: Homo sapiens similar to hypothetical |
| | | | protein LOC284701, transcript variant 1 (LOC642816), |
| | | | mRNA. |
| 360132 | LHFPL2 | 10184 | Homo sapiens lipoma HMGIC fusion partner-like 2 |
| | | | (LHFPL2), mRNA. |
| 1050025 | CR1 | 1378 | Homo sapiens complement component (3b/4b) receptor 1 |
| | | | (Knops blood group) (CR1), transcript variant F, mRNA. |
| 3840215 | FAM102A | 399665 | Homo sapiens family with sequence similarity 102, |
| | | | member A (FAM102A), transcript variant 1, mRNA. |
| 1850546 | FAM102A | 399665 | Homo sapiens family with sequence similarity 102, |
| | | | member A (FAM102A), transcript variant 1, mRNA. |
| 4220592 | CACNA2D3 | 55799 | Homo sapiens calcium channel, voltage-dependent, alpha |
| | | | 2/delta subunit 3 (CACNA2D3), mRNA. |
| 4070538 | TDP1 | 55775 | Homo sapiens tyrosyl-DNA phosphodiesterase 1 (TDP1), |
| | | | transcript variant 1, mRNA. |
| 6450326 | CHMP5 | 51510 | Homo sapiens chromatin modifying protein 5 (CHMP5), |
| | | | mRNA. |
| 4640427 | HIATL1 | 84641 | Homo sapiens hippocampus abundant transcript-like 1 |
| | | | (HIATL1), mRNA. |
| 6420594 | TIFA | 92610 | Homo sapiens TRAF-interacting protein with forkhead- |
| | | | associated domain (TIFA), mRNA. |
| 1300192 | ARL4C | 10123 | Homo sapiens ADP-ribosylation factor-like 4C (ARL4C), |
| | | | mRNA. |
| 5220767 | FLNB | 2317 | Homo sapiens filamin B, beta (actin binding protein 278) |
| | | | (FLNB), mRNA. |
| 6650482 | EPHA4 | 2043 | Homo sapiens EPH receptor A4 (EPHA4), mRNA. |
| 1070367 | C19orf59 | 199675 | Homo sapiens chromosome 19 open reading frame 59 |
| | | | (C19orf59), mRNA. |
| 6020523 | PPP2R2B | 5521 | Homo sapiens protein phosphatase 2 (formerly 2A), |
| | | | regulatory subunit B, beta isoform (PPP2R2B), transcript |
| | | | variant 4, mRNA. |
| 4260017 | CDK5RAP2 | 55755 | Homo sapiens CDK5 regulatory subunit associated protein |
| | | | 2 (CDK5RAP2), transcript variant 2, mRNA. |
| 3890609 | PLSCR1 | 5359 | Homo sapiens phospholipid scramblase 1 (PLSCR1), |
| | | | mRNA. |
| 1050292 | GP9 | 2815 | Homo sapiens glycoprotein IX (platelet) (GP9), mRNA. |
| 6480630 | ATP9A | 10079 | Homo sapiens ATPase, class II, type 9A (ATP9A), |
| | | | mRNA. |
| 870202 | TNFSF10 | 8743 | Homo sapiens tumor necrosis factor (ligand) superfamily, |
| | | | member 10 (TNFSF10), mRNA. |
| 3780382 | DKFZp761E198 | 91056 | Homo sapiens DKFZp761E198 protein (DKFZp761E198), |
| | | | mRNA. |
| 5560280 | SAMD14 | 201191 | Homo sapiens sterile alpha motif domain containing 14 |
| | | | (SAMD14), mRNA. |
| 4150692 | PARP14 | 54625 | Homo sapiens poly (ADP-ribose) polymerase family, |
| | | | member 14 (PARP14), mRNA. |
| 3310025 | OCIAD2 | 132299 | Homo sapiens OCIA domain containing 2 (OCIAD2), |
| | | | transcript variant 2, mRNA. |
| 5700735 | PARP9 | 83666 | Homo sapiens poly (ADP-ribose) polymerase family, |
| | | | member 9 (PARP9), mRNA. |
| 3140707 | PARP9 | 83666 | Homo sapiens poly (ADP-ribose) polymerase family, |
| | | | member 9 (PARP9), mRNA. |
| 1940132 | CD151 | 977 | Homo sapiens CD151 molecule (Raph blood group) |
| | | | (CD151), transcript variant 4, mRNA. |
| 4640270 | SIGLEC5 | 8778 | Homo sapiens sialic acid binding Ig-like lectin 5 |
| | | | (SIGLEC5), mRNA. |
| 2810471 | PTGDR | 5729 | Homo sapiens prostaglandin D2 receptor (DP) (PTGDR), |
| | | | mRNA. |
| 1030431 | ACSL1 | 2180 | Homo sapiens acyl-CoA synthetase long-chain family |
| | | | member 1 (ACSL1), mRNA. |
| 3360132 | SNX20 | 124460 | Homo sapiens sorting nexin 20 (SNX20), mRNA. |
| 1050068 | F2RL1 | 2150 | Homo sapiens coagulation factor II (thrombin) receptor- |
| | | | like 1 (F2RL1), mRNA. |
| 5810047 | F2RL1 | 2150 | Homo sapiens coagulation factor II (thrombin) receptor- |
| | | | like I (F2RL1), mRNA. |
| 5560075 | MFGE8 | 4240 | Homo sapiens milk fat globule-EGF factor 8 protein |
| | | | (MFGE8), mRNA. |
| 2470601 | IL1RN | 3557 | Homo sapiens interleukin 1 receptor antagonist (IL1RN), |
| | | | transcript variant 1, mRNA. |
| 3120474 | KIAA1671 | 85379 | PREDICTED: Homo sapiens KIAA1671 protein |
| | | | (KIAA1671), mRNA. |
| 6900521 | PHTF1 | 10745 | Homo sapiens putative homeodomain transcription factor |
| | | | 1 (PHTF1), mRNA. |
| 3190521 | MS4A1 | 931 | Homo sapiens membrane-spanning 4-domains, subfamily |
| | | | A, member 1 (MS4A1), transcript variant 1, mRNA. |
| 60719 | KCNJ2 | 3759 | Homo sapiens potassium inwardly-rectifying channel, |
| | | | subfamily J, member 2 (KCNJ2), mRNA. |
| 5340767 | CEACAM1 | 634 | Homo sapiens carcinoembryonic antigen-related cell |
| | | | adhesion molecule 1 (biliary glycoprotein) (CEACAM1), |
| | | | transcript variant 1, mRNA. |
| 3370577 | APP | 351 | Homo sapiens amyloid beta (A4) precursor protein (APP), |
| | | | transcript variant 3, mRNA. |
| 650110 | APP | 351 | Homo sapiens amyloid beta (A4) precursor protein (APP), |
| | | | transcript variant 3, mRNA. |
| 450189 | XAF1 | 54739 | Homo sapiens XIAP associated factor 1 (XAF1), transcript |
| | | | variant 2, mRNA. |
| 7610440 | XAF1 | 54739 | Homo sapiens XIAP associated factor 1 (XAF1), transcript |
| | | | variant 2, mRNA. |
| 1940373 | ZNF831 | 128611 | Homo sapiens zinc finger protein 831 (ZNF831), mRNA. |
| 6270040 | ZNF831 | 128611 | Homo sapiens zinc finger protein 831 (ZNF831), mRNA. |
| 2710646 | MSRB2 | 22921 | Homo sapiens methionine sulfoxide reductase B2 |
| | | | (MSRB2), mRNA. |
| 6380411 | IKZF3 | 22806 | Homo sapiens IKAROS family zinc finger 3 (Aiolos) |
| | | | (IKZF3), transcript variant 1, mRNA. |
| 380050 | RHBDF2 | 79651 | Homo sapiens rhomboid 5 homolog 2 (Drosophila) |
| | | | (RHBDF2), transcript variant 1, mRNA. |
| 6200019 | KLRB1 | 3820 | Homo sapiens killer cell lectin-like receptor subfamily B, |
| | | | member 1 (KLRB1), mRNA. |
| 4810768 | S1PR1 | 1901 | Homo sapiens sphingosine-1-phosphate receptor 1 |
| | | | (S1PR1), mRNA. |
| 3310341 | FYB | 2533 | Homo sapiens FYN binding protein (FYB-120/130) |
| | | | (FYB), transcript variant 1, mRNA. |
| 4260725 | CD79B | 974 | Homo sapiens CD79B antigen (immunoglobulin- |
| | | | associated beta) (CD79B), transcript variant 2, mRNA. |
| 990470 | LOC651738 | 651738 | PREDICTED: Homo sapiens hypothetical LOC651738 |
| | | | (LOC651738), mRNA. |
| 670603 | PLAGL1 | 5325 | Homo sapiens pleiomorphic adenoma gene-like 1 |
| | | | (PLAGL1), transcript variant 3, mRNA. |
| 3310376 | TIMM10 | 26519 | Homo sapiens translocase of inner mitochondrial |
| | | | membrane 10 homolog (yeast) (TIMM10), nuclear gene |
| | | | encoding mitochondrial protein, mRNA. |
| 150626 | LOC641710 | 641710 | PREDICTED: Homo sapiens hypothetical protein |
| | | | LOC641710, transcript variant 1 (LOC641710), mRNA. |
| 7610598 | TRAF5 | 7188 | Homo sapiens TNF receptor-associated factor 5 (TRAF5), |
| | | | transcript variant 1, mRNA. |
| 7330392 | TAP1 | 6890 | Homo sapiens transporter 1, ATP-binding cassette, sub- |
| | | | family B (MDR/TAP) (TAP1), mRNA. |
| 6770070 | FCRL2 | 79368 | Homo sapiens Fc receptor-like 2 (FCRL2), transcript |
| | | | variant 2, mRNA. |
| 7000474 | RALGAPA1 | 253959 | Homo sapiens Ral GTPase activating protein, alpha |
| | | | subunit 1 (catalytic) (RALGAPA1), transcript variant 2, |
| | | | mRNA. |
| 7400025 | MTP18 | 51537 | Homo sapiens mitochondrial protein 18 kDa (MTP18), |
| | | | nuclear gene encoding mitochondrial protein, transcript |
| | | | variant 1, mRNA. |
| 1010170 | BACH1 | 571 | Homo sapiens BTB and CNC homology 1, basic leucine |
| | | | zipper transcription factor 1 (BACH1), transcript variant 3, |
| | | | mRNA. |
| 6280301 | LOC643313 | 643313 | PREDICTED: Homo sapiens similar to hypothetical |
| | | | protein LOC284701, transcript variant 3 (LOC643313), |
| | | | mRNA. |
| 1570358 | APOL2 | 23780 | Homo sapiens apolipoprotein L, 2 (APOL2), transcript |
| | | | variant beta, mRNA. |
| 3370066 | APOL2 | 23780 | Homo sapiens apolipoprotein L, 2 (APOL2), transcript |
| | | | variant beta, mRNA. |
| 2140382 | PASK | 23178 | Homo sapiens PAS domain containing serine/threonine |
| | | | kinase (PASK), mRNA. |
| 6510707 | FER1L3 | 26509 | Homo sapiens fer-1-like 3, myoferlin (C. elegans) |
| | | | (FER1L3), transcript variant 1, mRNA. |
| 3800019 | NCR3 | 259197 | Homo sapiens natural cytotoxicity triggering receptor 3 |
| | | | (NCR3), mRNA. |
| 4210753 | CD8B | 926 | Homo sapiens CD8b molecule (CD8B), transcript variant |
| | | | 5, mRNA. |
| 3830433 | | | BX096301 Soares retina N2b5HR Homo sapiens cDNA |
| | | | clone IMAGp998C16444, mRNA sequence |
| 1710553 | HSPA6 | 3310 | Homo sapiens heat shock 70 kDa protein 6 (HSP70B′) |
| | | | (HSPA6), mRNA. |
| 5290369 | USP32 | 84669 | Homo sapiens ubiquitin specific peptidase 32 (USP32), |
| | | | mRNA. |
| 4150543 | FPR2 | 2358 | Homo sapiens formyl peptide receptor 2 (FPR2), transcript |
| | | | variant 2, mRNA. |
| 2030678 | HIST2H2AB | 317772 | Homo sapiens histone cluster 2, H2ab (HIST2H2AB), |
| | | | mRNA. |
| 770253 | KIAA1618 | 57714 | PREDICTED: Homo sapiens KIAA1618 (KIAA1618), |
| | | | mRNA. |
| 50136 | CMTM5 | 116173 | Homo sapiens CKLF-like MARVEL transmembrane |
| | | | domain containing 5 (CMTM5), transcript variant 3, |
| | | | mRNA. |
| 2600739 | VRK1 | 7443 | Homo sapiens vaccinia related kinase 1 (VRK1), mRNA. |
| 4290619 | CREB5 | 9586 | Homo sapiens cAMP responsive element binding protein 5 |
| | | | (CREB5), transcript variant 4, mRNA. |
| 3420026 | FAS | 355 | Homo sapiens Fas (TNF receptor superfamily, member 6) |
| | | | (FAS), transcript variant 7, mRNA. |
| 2030209 | MTF1 | 4520 | Homo sapiens metal-regulatory transcription factor 1 |
| | | | (MTF1), mRNA. |
| 1260192 | QKI | 9444 | Homo sapiens quaking homolog, KH domain RNA |
| | | | binding (mouse) (QKI), transcript variant 2, mRNA. |
| 7160066 | C20orf196 | 149840 | Homo sapiens chromosome 20 open reading frame 196 |
| | | | (C20orf196), mRNA. |
| 7050382 | CASP1 | 834 | Homo sapiens caspase 1, apoptosis-related cysteine |
| | | | peptidase (interleukin 1, beta, convertase) (CASP1), |
| | | | transcript variant delta, mRNA. |
| 5310437 | MYL9 | 10398 | Homo sapiens myosin, light chain 9, regulatory (MYL9), |
| | | | transcript variant 1, mRNA. |
| 2480327 | MYL9 | 10398 | Homo sapiens myosin, light chain 9, regulatory (MYL9), |
| | | | transcript variant 1, mRNA. |
| 1580750 | LOC100130828 | 100130828 | PREDICTED: Homo sapiens hypothetical LOC100130828 |
| | | | (LOC100130828), mRNA. |
| 5340468 | ITGA2B | 3674 | Homo sapiens integrin, alpha 2b (platelet glycoprotein IIb |
| | | | of IIb/IIIa complex, antigen CD41B) (ITGA2B), mRNA. |
| 1010246 | IFI6 | 2537 | Homo sapiens interferon, alpha-inducible protein 6 (IFI6), |
| | | | transcript variant 2, mRNA. |
| 2140020 | LIMK2 | 3985 | Homo sapiens LIM domain kinase 2 (LIMK2), transcript |
| | | | variant 2b, mRNA. |
| 3390048 | LIMK2 | 3985 | Homo sapiens LIM domain kinase 2 (LIMK2), transcript |
| | | | variant 2b, mRNA. |
| 5910632 | SMARCD3 | 6604 | Homo sapiens SWI/SNF related, matrix associated, actin |
| | | | dependent regulator of chromatin, subfamily d, member 3 |
| | | | (SMARCD3), transcript variant 2, mRNA. |
| 5570180 | LCK | 3932 | Homo sapiens lymphocyte-specific protein tyrosine kinase |
| | | | (LCK), mRNA. |
| 3170136 | SAMD9L | 219285 | Homo sapiens sterile alpha motif domain containing 9-like |
| | | | (SAMD9L), mRNA. |
| 5260349 | NGFRAP1 | 27018 | Homo sapiens nerve growth factor receptor (TNFRSF16) |
| | | | associated protein 1 (NGFRAP1), transcript variant 1, |
| | | | mRNA. |
| 6860220 | NGFRAP1 | 27018 | Homo sapiens nerve growth factor receptor (TNFRSF16) |
| | | | associated protein 1 (NGFRAP1), transcript variant 1, |
| | | | mRNA. |
| 4760601 | LOC728014 | 728014 | PREDICTED: Homo sapiens similar to huntingtin |
| | | | interacting protein 1 related (LOC728014), mRNA. |
| 3940041 | SIRPG | 55423 | Homo sapiens signal-regulatory protein gamma (SIRPG), |
| | | | transcript variant 3, mRNA. |
| 4900747 | C11orf75 | 56935 | Homo sapiens chromosome 11 open reading frame 75 |
| | | | (C11orf75), mRNA. |
| 2030148 | WDR54 | 84058 | Homo sapiens WD repeat domain 54 (WDR54), mRNA. |
| 6400332 | CNIH4 | 29097 | Homo sapiens cornichon homolog 4 (Drosophila) |
| | | | (CNIH4), mRNA. |
| 3170703 | LY9 | 4063 | Homo sapiens lymphocyte antigen 9 (LY9), transcript |
| | | | variant 2, mRNA. |
| 1240358 | RAB20 | 55647 | Homo sapiens RAB20, member RAS oncogene family |
| | | | (RAB20), mRNA. |
| 1990300 | SOCS1 | 8651 | Homo sapiens suppressor of cytokine signaling 1 |
| | | | (SOCS1), mRNA. |
| 7040477 | PLOD2 | 5352 | Homo sapiens procollagen-lysine, 2-oxoglutarate 5- |
| | | | dioxygenase 2 (PLOD2), transcript variant 2, mRNA. |
| 7200338 | BMP2K | 55589 | Homo sapiens BMP2 inducible kinase (BMP2K), |
| | | | transcript variant 1, mRNA. |
| 1470685 | ITGB3 | 3690 | Homo sapiens integrin, beta 3 (platelet glycoprotein IIIa, |
| | | | antigen CD61) (ITGB3), mRNA. |
| 630315 | DHRS9 | 10170 | Homo sapiens dehydrogenase/reductase (SDR family) |
| | | | member 9 (DHRS9), transcript variant 1, mRNA. |
| 3800707 | XPO4 | 64328 | Homo sapiens exportin 4 (XPO4), mRNA. |
| 620167 | FCRL3 | 115352 | Homo sapiens Fc receptor-like 3 (FCRL3), mRNA. |
| 540382 | RSAD1 | 55316 | Homo sapiens radical S-adenosyl methionine domain |
| | | | containing 1 (RSAD1), mRNA. |
| 6760286 | | | RST24587 Athersys RAGE Library Homo sapiens cDNA, |
| | | | mRNA sequence |
| 4290148 | HIST2H2AA4 | 723790 | Homo sapiens histone cluster 2, H2aa4 (HIST2H2AA4), |
| | | | mRNA. |
| 1780440 | CD79A | 973 | Homo sapiens CD79a molecule, immunoglobulin- |
| | | | associated alpha (CD79A), transcript variant 1, mRNA. |
| 3130220 | TMEM158 | 25907 | Homo sapiens transmembrane protein 158 (TMEM158), |
| | | | mRNA. |
| 3290292 | LAP3 | 51056 | Homo sapiens leucine aminopeptidase 3 (LAP3), mRNA. |
| 830341 | TBRG4 | 9238 | Homo sapiens transforming growth factor beta regulator 4 |
| | | | (TBRG4), transcript variant 2, mRNA. |
| 4900435 | TNFSF13B | 10673 | Homo sapiens tumor necrosis factor (ligand) superfamily, |
| | | | member 13b (TNFSF13B), transcript variant 1, mRNA. |
| 1240553 | SESN1 | 27244 | Homo sapiens sestrin 1 (SESN1), mRNA. |
| 4850592 | P2RY14 | 9934 | Homo sapiens purinergic receptor P2Y, G-protein coupled, |
| | | | 14 (P2RY14), transcript variant 2, mRNA. |
| 4610241 | P2RY14 | 9934 | Homo sapiens purinergic receptor P2Y, G-protein coupled, |
| | | | 14 (P2RY14), transcript variant 2, mRNA. |
| 1340364 | P2RY14 | 9934 | Homo sapiens purinergic receptor P2Y, G-protein coupled, |
| | | | 14 (P2RY14), transcript variant 2, mRNA. |
| 6560072 | | | EST366269 MAGE resequences, MAGC Homo sapiens |
| | | | cDNA, mRNA sequence |
| 6510170 | IFIT3 | 3437 | Homo sapiens interferon-induced protein with |
| | | | tetratricopeptide repeats 3 (IFIT3), mRNA. |
| 1500280 | IFIT3 | 3437 | Homo sapiens interferon-induced protein with |
| | | | tetratricopeptide repeats 3 (IFIT3), mRNA. |
| 4180544 | ROPN1L | 83853 | Homo sapiens ropporin 1-like (ROPN1L), mRNA. |
| 3290044 | TTC27 | 55622 | Homo sapiens tetratricopeptide repeat domain 27 (TTC27), |
| | | | mRNA. |
| 610358 | PGS1 | 9489 | Homo sapiens phosphatidylglycerophosphate synthase 1 |
| | | | (PGS1), mRNA. |
| 2190184 | PRSS23 | 11098 | Homo sapiens protease, serine, 23 (PRSS23), mRNA. |
| 1400689 | JAM3 | 83700 | Homo sapiens junctional adhesion molecule 3 (JAM3), |
| | | | mRNA. |
| 7050458 | DBP | 1628 | Homo sapiens D site of albumin promoter (albumin D- |
| | | | box) binding protein (DBP), mRNA. |
| 2190121 | GGA2 | 23062 | Homo sapiens golgi associated, gamma adaptin ear |
| | | | containing, ARF binding protein 2 (GGA2), mRNA. |
| 7570575 | LOC642788 | 642788 | PREDICTED: Homo sapiens hypothetical protein |
| | | | LOC642788, transcript variant 2 (LOC642788), mRNA. |
| 540390 | ALPK1 | 80216 | Homo sapiens alpha-kinase 1 (ALPK1), mRNA. |
| 5080601 | COBLL1 | 22837 | Homo sapiens COBL-like 1 (COBLL1), mRNA. |
| 6860639 | ATPGD1 | 57571 | Homo sapiens ATP-grasp domain containing 1 |
| | | | (ATPGD1), transcript variant 2, mRNA. |
| 2070369 | TREML1 | 340205 | Homo sapiens triggering receptor expressed on myeloid |
| | | | cells-like 1 (TREML1), mRNA. |
| 4890092 | TSEN54 | 283989 | Homo sapiens tRNA splicing endonuclease 54 homolog |
| | | | (S. cerevisiae) (TSEN54), mRNA. |
| 4120053 | PFKFB3 | 5209 | Homo sapiens 6-phosphofructo-2-kinase/fructose-2,6- |
| | | | biphosphatase 3 (PFKFB3), mRNA. |
| 2940484 | GNB4 | 59345 | Homo sapiens guanine nucleotide binding protein (G |
| | | | protein), beta polypeptide 4 (GNB4), mRNA. |
| 4150270 | ANKRD22 | 118932 | Homo sapiens ankyrin repeat domain 22 (ANKRD22), |
| | | | mRNA. |
| 6420008 | PROS1 | 5627 | Homo sapiens protein S (alpha) (PROS1), mRNA. |
| 4230328 | CALD1 | 800 | Homo sapiens caldesmon 1 (CALD1), transcript variant 3, |
| | | | mRNA. |
| 50706 | CD40LG | 959 | Homo sapiens CD40 ligand (CD4OLG), mRNA. |
| 2140524 | HIST1H3D | 8351 | Homo sapiens histone cluster 1, H3d (HIST1H3D), |
| | | | mRNA. |
| 7610113 | SLC26A8 | 116369 | Homo sapiens solute carrier family 26, member 8 |
| | | | (SLC26A8), transcript variant 2, mRNA. |
| 5360626 | SLC26A8 | 116369 | Homo sapiens solute carrier family 26, member 8 |
| | | | (SLC26A8), transcript variant 2, mRNA. |
| 2070170 | UBE2L6 | 9246 | Homo sapiens ubiquitin-conjugating enzyme E2L 6 |
| | | | (UBE2L6), transcript variant 1, mRNA. |
| 7000368 | UBE2L6 | 9246 | Homo sapiens ubiquitin-conjugating enzyme E2L 6 |
| | | | (UBE2L6), transcript variant 1, mRNA. |
| 6280092 | SLC25A23 | 79085 | Homo sapiens solute carrier family 25 (mitochondrial |
| | | | carrier; phosphate carrier), member 23 (SLC25A23), |
| | | | nuclear gene encoding mitochondrial protein, mRNA. |
| 770088 | DTX3L | 151636 | Homo sapiens deltex 3-like (Drosophila) (DTX3L), |
| | | | mRNA. |
| 1980431 | DOK3 | 79930 | Homo sapiens docking protein 3 (DOK3), mRNA. |
| 5310711 | FAM160B1 | 57700 | Homo sapiens family with sequence similarity 160, |
| | | | member B1 (FAM160B1), transcript variant 2, mRNA. |
| 4780068 | SULT1B1 | 27284 | Homo sapiens sulfotransferase family, cytosolic, 1B, |
| | | | member 1 (SULT1B1), mRNA. |
| 5670465 | ADM | 133 | Homo sapiens adrenomedullin (ADM), mRNA. |
| 620091 | LOC730234 | 730234 | PREDICTED: Homo sapiens misc_RNA (LOC730234), |
| | | | miscRNA. |
| 1820592 | HIST2H2AA3 | 8337 | Homo sapiens histone cluster 2, H2aa3 (HIST2H2AA3), |
| | | | mRNA. |
| 610451 | HIST2H2AA3 | 8337 | Homo sapiens histone cluster 2, H2aa3 (HIST2H2AA3), |
| | | | mRNA. |
| 5220142 | LIMK2 | 3985 | Homo sapiens LIM domain kinase 2 (LIMK2), transcript |
| | | | variant 2a, mRNA. |
| 1050008 | MMRN1 | 22915 | Homo sapiens multimerin 1 (MMRN1), mRNA. |
| 670528 | GYG1 | 2992 | Homo sapiens glycogenin 1 (GYG1), mRNA. |
| 6960397 | EPHX2 | 2053 | Homo sapiens epoxide hydrolase 2, cytoplasmic (EPHX2), |
| | | | mRNA. |
| 2570041 | WASF3 | 10810 | Homo sapiens WAS protein family, member 3 (WASF3), |
| | | | mRNA. |
| 2350747 | ABLIM3 | 22885 | Homo sapiens actin binding LIM protein family, member 3 |
| | | | (ABLIM3), mRNA. |
| 6060053 | LILRA6 | 79168 | Homo sapiens leukocyte immunoglobulin-like receptor, |
| | | | subfamily A (with TM domain), member 6 (LILRA6), |
| | | | mRNA. |
| 2350278 | PNOC | 5368 | Homo sapiens prepronociceptin (PNOC), mRNA. |
| 4570725 | LOC653604 | 653604 | PREDICTED: Homo sapiens similar to H3 histone, family |
| | | | 2 isoform 2 (LOC653604), mRNA. |
| 3420523 | RHBDF2 | 79651 | Homo sapiens rhomboid 5 homolog 2 (Drosophila) |
| | | | (RHBDF2), transcript variant 2, mRNA. |
| 3360500 | EGF | 1950 | Homo sapiens epidermal growth factor (beta-urogastrone) |
| | | | (EGF), mRNA. |
| 7000270 | PYHIN1 | 149628 | Homo sapiens pyrin and HIN domain family, member 1 |
| | | | (PYHIN1), transcript variant b2, mRNA. |
| 4200754 | ST3GAL4 | 6484 | Homo sapiens ST3 beta-galactoside alpha-2,3- |
| | | | sialyltransferase 4 (ST3GAL4), mRNA. |
| 110196 | TMEM19 | 55266 | Homo sapiens transmembrane protein 19 (TMEM19), |
| | | | mRNA. |
| 3710086 | DHRS12 | 79758 | Homo sapiens dehydrogenase/reductase (SDR family) |
| | | | member 12 (DHRS12), transcript variant 2, mRNA. |
| 6980133 | | | HUMGS0004661 Human adult (K. Okubo) Homo sapiens |
| | | | cDNA 3, mRNA sequence |
| 6590646 | FAM26F | 441168 | Homo sapiens family with sequence similarity 26, member |
| | | | F (FAM26F), mRNA. |
| 3780193 | FCRLA | 84824 | Homo sapiens Fc receptor-like A (FCRLA), mRNA. |
| 5290465 | CASP7 | 840 | Homo sapiens caspase 7, apoptosis-related cysteine |
| | | | peptidase (CASP7), transcript variant gamma, mRNA. |
| 2230241 | F13A1 | 2162 | Homo sapiens coagulation factor XIII, A1 polypeptide |
| | | | (F13A1), mRNA. |
| 60524 | DENND1A | 57706 | Homo sapiens DENN/MADD domain containing 1A |
| | | | (DENND1A), transcript variant 2, mRNA. |
| 6620161 | SPIB | 6689 | Homo sapiens Spi-B transcription factor (Spi-1/PU.1 |
| | | | related) (SPIB), mRNA. |
| 2490463 | MXI1 | 4601 | Homo sapiens MAX interactor 1 (MXI1), transcript |
| | | | variant 2, mRNA. |
| 3370400 | LOC643313 | 643313 | PREDICTED: Homo sapiens similar to hypothetical |
| | | | protein LOC284701, transcript variant 1 (LOC643313), |
| | | | mRNA. |
| 4260632 | PDZD4 | 57595 | Homo sapiens PDZ domain containing 4 (PDZD4), |
| | | | mRNA. |
| 4900239 | CD274 | 29126 | Homo sapiens CD274 molecule (CD274), mRNA. |
| 940274 | GK | 2710 | Homo sapiens glycerol kinase (GK), transcript variant 2, |
| | | | mRNA. |
| 3940189 | JAK2 | 3717 | Homo sapiens Janus kinase 2 (a protein tyrosine kinase) |
| | | | (JAK2), mRNA. |
| 3850630 | HOMER2 | 9455 | Homo sapiens homer homolog 2 (Drosophila) (HOMER2), |
| | | | transcript variant 3, mRNA. |
| 6380672 | CA4 | 762 | Homo sapiens carbonic anhydrase IV (CA4), mRNA. |
| 6650242 | IFITM3 | 10410 | Homo sapiens interferon induced transmembrane protein 3 |
| | | | (1-8U) (IFITM3), mRNA. |
| 2470017 | LILRB3 | 11025 | Homo sapiens leukocyte immunoglobulin-like receptor, |
| | | | subfamily B (with TM and ITIM domains), member 3 |
| | | | (LILRB3), transcript variant 2, mRNA. |
| 1820750 | STAT1 | 6772 | Homo sapiens signal transducer and activator of |
| | | | transcription 1, 91 kDa (STAT1), transcript variant alpha, |
| | | | mRNA. |
| 4810187 | STAT1 | 6772 | Homo sapiens signal transducer and activator of |
| | | | transcription 1, 91 kDa (STAT1), transcript variant alpha, |
| | | | mRNA. |
| 7210672 | OR2AG1 | 144125 | Homo sapiens olfactory receptor, family 2, subfamily AG, |
| | | | member 1 (OR2AG1), mRNA. |
| 1260066 | | | Homo sapiens cDNA: FLJ21199 fis, clone COL00235 |
| 6660162 | LRG1 | 116844 | Homo sapiens leucine-rich alpha-2-glycoprotein 1 |
| | | | (LRG1), mRNA. |
| 3130307 | TYMP | 1890 | Homo sapiens thymidine phosphorylase (TYMP), |
| | | | transcript variant 1, mRNA. |
| 6960022 | Sep-05 | 5413 | Homo sapiens septin 5 (SEPT5), mRNA. |
| 1450484 | HIST1H2BE | 8344 | Homo sapiens histone cluster 1, H2be (HIST1H2BE), |
| | | | mRNA. |
| 990349 | MCTP2 | 55784 | Homo sapiens multiple C2 domains, transmembrane 2 |
| | | | (MCTP2), mRNA. |
| 2340577 | AQP10 | 89872 | Homo sapiens aquaporin 10 (AQP10), mRNA. |
| 7400372 | WSB1 | 26118 | Homo sapiens WD repeat and SOCS box-containing 1 |
| | | | (WSB1), transcript variant 3, mRNA. |
| 3800241 | CDH6 | 1004 | Homo sapiens cadherin 6, type 2, K-cadherin (fetal kidney) |
| | | | (CDH6), mRNA. |
| 6100022 | HIST2H2AC | 8338 | Homo sapiens histone cluster 2, H2ac (HIST2H2AC), |
| | | | mRNA. |
| 5550474 | RAB24 | 53917 | Homo sapiens RAB24, member RAS oncogene family |
| | | | (RAB24), transcript variant 2, mRNA. |
| 5270520 | FAIM3 | 9214 | Homo sapiens Fas apoptotic inhibitory molecule 3 |
| | | | (FAIM3), mRNA. |
| 770348 | LOC649260 | 649260 | PREDICTED: Homo sapiens similar to LIM and senescent |
| | | | cell antigen-like domains 1 (LOC649260), mRNA. |
| 4610066 | TMEM194A | 23306 | Homo sapiens transmembrane protein 194A |
| | | | (TMEM194A), transcript variant 2, mRNA. |
| 7510537 | SCO2 | 9997 | Homo sapiens SCO cytochrome oxidase deficient homolog |
| | | | 2 (yeast) (SCO2), nuclear gene encoding mitochondrial |
| | | | protein, mRNA. |
| 380082 | LOC646144 | 646144 | PREDICTED: Homo sapiens similar to tousled-like kinase |
| | | | 2, transcript variant 2 (LOC646144), mRNA. |
| 1470470 | GPR97 | 222487 | Homo sapiens G protein-coupled receptor 97 (GPR97), |
| | | | mRNA. |
| 5260424 | GPR97 | 222487 | Homo sapiens G protein-coupled receptor 97 (GPR97), |
| | | | mRNA. |
| 4230441 | GPR97 | 222487 | Homo sapiens G protein-coupled receptor 97 (GPR97), |
| | | | mRNA. |
| 1450301 | SLAMF6 | 114836 | Homo sapiens SLAM family member 6 (SLAMF6), |
| | | | mRNA. |
| 2710152 | ODF3B | 440836 | Homo sapiens outer dense fiber of sperm tails 3B |
| | | | (ODF3B), mRNA. |
| 2370743 | LOC100132858 | 100132858 | PREDICTED: Homo sapiens hypothetical protein |
| | | | LOC100132858 (LOC100132858), mRNA. |
| 6550259 | CD151 | 977 | Homo sapiens CD151 molecule (Raph blood group) |
| | | | (CD151), transcript variant 5, mRNA. |
| 7400152 | P2RY10 | 27334 | Homo sapiens purinergic receptor P2Y, G-protein coupled, |
| | | | 10 (P2RY10), transcript variant 1, mRNA. |
| 6520180 | IL18RAP | 8807 | Homo sapiens interleukin 18 receptor accessory protein |
| | | | (IL18RAP), mRNA. |
| 4890598 | BRSK1 | 84446 | Homo sapiens BR serine/threonine kinase 1 (BRSK1), |
| | | | mRNA. |
| 6760593 | OSBPL10 | 114884 | Homo sapiens oxysterol binding protein-like 10 |
| | | | (OSBPL10), mRNA. |
| 1440750 | S1PR5 | 53637 | Homo sapiens sphingosine-1-phosphate receptor 5 |
| | | | (S1PR5), mRNA. |
| 1850754 | LOC644642 | 644642 | PREDICTED: Homo sapiens hypothetical protein |
| | | | LOC644642 (LOC644642), mRNA. |
| 3140487 | BMX | 660 | Homo sapiens BMX non-receptor tyrosine kinase (BMX), |
| | | | mRNA. |
| 360343 | RHOBTB1 | 9886 | Homo sapiens Rho-related BTB domain containing 1 |
| | | | (RHOBTB1), transcript variant 2, mRNA. |
| 3990224 | TNFRSF25 | 8718 | Homo sapiens tumor necrosis factor receptor superfamily, |
| | | | member 25 (TNFRSF25), transcript variant 10, mRNA. |
| 2570079 | STAT1 | 6772 | Homo sapiens signal transducer and activator of |
| | | | transcription 1, 91 kDa (STAT1), transcript variant beta, |
| | | | mRNA. |
| 1570424 | WNK1 | 65125 | Homo sapiens WNK lysine deficient protein kinase 1 |
| | | | (WNK1), mRNA. |
| 4010681 | LOC374443 | 374443 | Homo sapiens CLR pseudogene (LOC374443), non- |
| | | | coding RNA. XR_001315 |
| 5360243 | ITGB5 | 3693 | Homo sapiens integrin, beta 5 (ITGB5), mRNA. |
| | | | XM_944688 XM_944693 |
| 2490411 | ITGB5 | 3693 | Homo sapiens integrin, beta 5 (ITGB5), mRNA. |
| | | | XM_944688 XM_944693 |
| 2650114 | ITGB5 | 3693 | Homo sapiens integrin, beta 5 (ITGB5), mRNA. |
| | | | XM_944688 XM_944693 |
| 520086 | FCGR1A | 2209 | Homo sapiens Fc fragment of IgG, high affinity Ia, |
| | | | receptor (CD64) (FCGR1A), mRNA. |
| 2570300 | IFI44 | 10561 | Homo sapiens interferon-induced protein 44 (IFI44), |
| | | | mRNA. |
| 130161 | LCK | 3932 | Homo sapiens lymphocyte-specific protein tyrosine kinase |
| | | | (LCK), transcript variant 1, mRNA. |
| 630619 | HPSE | 10855 | Homo sapiens heparanase (HPSE), mRNA. |
| 6620689 | MTHFD2 | 10797 | Homo sapiens methylenetetrahydrofolate dehydrogenase |
| | | | (NADP+ dependent) 2, methenyltetrahydrofolate |
| | | | cyclohydrolase (MTHFD2), nuclear gene encoding |
| | | | mitochondrial protein, transcript variant 2, mRNA. |
| 6290672 | LOC728417 | 728417 | PREDICTED: Homo sapiens hypothetical protein |
| | | | LOC728417 (LOC728417), mRNA. |
| 2570482 | LOC728417 | 728417 | PREDICTED: Homo sapiens hypothetical protein |
| | | | LOC728417 (LOC728417), mRNA. |
| 5560471 | FCAR | 2204 | Homo sapiens Fc fragment of IgA, receptor for (FCAR), |
| | | | transcript variant 10, mRNA. |
| 3420259 | MIR21 | 406991 | Homo sapiens microRNA 21 (MIR21), microRNA. |
| 3850246 | HOPX | 84525 | Homo sapiens HOP homeobox (HOPX), transcript variant |
| | | | 3, mRNA. |
| 6860164 | CLEC1B | 51266 | Homo sapiens C-type lectin domain family 1, member B |
| | | | (CLEC1B), transcript variant 1, mRNA. |
| 3610274 | PJA1 | 64219 | Homo sapiens praja ring finger 1 (PJA1), transcript variant |
| | | | 2, mRNA. |
| 2030170 | CARD16 | 114769 | Homo sapiens caspase recruitment domain family, member |
| | | | 16 (CARD16), transcript variant 2, mRNA. |
| 870193 | SP140 | 11262 | Homo sapiens SP140 nuclear body protein (SP140), |
| | | | transcript variant 2, mRNA. |
| 6840390 | FBXL16 | 146330 | Homo sapiens F-box and leucine-rich repeat protein 16 |
| | | | (FBXL16), mRNA. |
| 5570039 | LOC728744 | 728744 | PREDICTED: Homo sapiens hypothetical LOC728744 |
| | | | (LOC728744), mRNA. |
| 4780678 | ZAP70 | 7535 | Homo sapiens zeta-chain (TCR) associated protein kinase |
| | | | 70 kDa (ZAP70), transcript variant 1, mRNA. |
| 5390204 | SCARF1 | 8578 | Homo sapiens scavenger receptor class F, member 1 |
| | | | (SCARF1), transcript variant 3, mRNA. |
| 5310634 | FASN | 2194 | Homo sapiens fatty acid synthase (FASN), mRNA. |
| 7040181 | PFKFB2 | 5208 | Homo sapiens 6-phosphofructo-2-kinase/fructose-2,6- |
| | | | biphosphatase 2 (PFKFB2), transcript variant 2, mRNA. |
| 5900543 | Sep-04 | 5414 | Homo sapiens septin 4 (SEPT4), transcript variant 3, |
| | | | mRNA. |
| 3710068 | WARS | 7453 | Homo sapiens tryptophanyl-tRNA synthetase (WARS), |
| | | | transcript variant 2, mRNA. |
| 3180528 | MMP9 | 4318 | Homo sapiens matrix metallopeptidase 9 (gelatinase B, |
| | | | 92 kDa gelatinase, 92 kDa type IV collagenase) (MMP9), |
| | | | mRNA. |
| 6130075 | LOC391769 | 391769 | PREDICTED: Homo sapiens similar to HIStone family |
| | | | member (his-72) (LOC391769), mRNA. |
| 2650156 | TAP2 | 6891 | Homo sapiens transporter 2, ATP-binding cassette, sub- |
| | | | family B (MDR/TAP) (TAP2), transcript variant 2, |
| | | | mRNA. |
| 460463 | SMARCD3 | 6604 | Homo sapiens SWI/SNF related, matrix associated, actin |
| | | | dependent regulator of chromatin, subfamily d, member 3 |
| | | | (SMARCD3), transcript variant 1, mRNA. |
| 1260133 | LOC642334 | 642334 | PREDICTED: Homo sapiens similar to hypothetical |
| | | | protein LOC284701, transcript variant 1 (LOC642334), |
| | | | mRNA. |
| 4860753 | ASGR2 | 433 | Homo sapiens asialoglycoprotein receptor 2 (ASGR2), |
| | | | transcript variant 3, mRNA. |
| 3800576 | ASGR2 | 433 | Homo sapiens asialoglycoprotein receptor 2 (ASGR2), |
| | | | transcript variant 3, mRNA. |
| 2600747 | IFIT2 | 3433 | Homo sapiens interferon-induced protein with |
| | | | tetratricopeptide repeats 2 (IFIT2), mRNA. |
| 2480450 | GALM | 130589 | Homo sapiens galactose mutarotase (aldose 1-epimerase) |
| | | | (GALM), mRNA. |
| 1660246 | GNB4 | 59345 | Homo sapiens guanine nucleotide binding protein (G |
| | | | protein), beta polypeptide 4 (GNB4), mRNA. |
| 5820491 | MAP7 | 9053 | Homo sapiens microtubule-associated protein 7 (MAP7), |
| | | | mRNA. |
| 1980524 | GBP4 | 115361 | Homo sapiens guanylate binding protein 4 (GBP4), |
| | | | mRNA. |
| 20075 | NDUFAF3 | 25915 | Homo sapiens NADH dehydrogenase (ubiquinone) 1 alpha |
| | | | subcomplex, assembly factor 3 (NDUFAF3), nuclear gene |
| | | | encoding mitochondrial protein, transcript variant 4, |
| | | | mRNA. |
| 3290170 | PAAF1 | 80227 | Homo sapiens proteasomal ATPase-associated factor 1 |
| | | | (PAAF1), mRNA. |
| 5270674 | GPR68 | 8111 | Homo sapiens G protein-coupled receptor 68 (GPR68), |
| | | | mRNA. |
| 4570441 | IFIH1 | 64135 | Homo sapiens interferon induced with helicase C domain 1 |
| | | | (IFIH1), mRNA. |
| 5810689 | PRRG4 | 79056 | Homo sapiens proline rich Gla (G-carboxyglutamic acid) 4 |
| | | | (transmembrane) (PRRG4), mRNA. |
| 3870170 | LOC728093 | 728093 | PREDICTED: Homo sapiens misc_RNA (LOC728093), |
| | | | Partial miscRNA. |
| 3420128 | AP3M2 | 10947 | Homo sapiens adaptor-related protein complex 3, mu 2 |
| | | | subunit (AP3M2), mRNA. |
| 7650440 | BACH2 | 60468 | Homo sapiens BTB and CNC homology 1, basic leucine |
| | | | zipper transcription factor 2 (BACH2), mRNA. |
| 70753 | C9orf123 | 90871 | Homo sapiens chromosome 9 open reading frame 123 |
| | | | (C9orf123), mRNA. |
| 7100703 | LOC100132287 | 100132287 | PREDICTED: Homo sapiens misc_RNA |
| | | | (LOC100132287), miscRNA. |
| 6220739 | GRAMD1B | 57476 | Homo sapiens GRAM domain containing 1B |
| | | | (GRAMD1B), mRNA. |
| 5890470 | CCR6 | 1235 | Homo sapiens chemokine (C-C motif) receptor 6 (CCR6), |
| | | | transcript variant 2, mRNA. |
| 540484 | MGC16384 | 114130 | PREDICTED: Homo sapiens hypothetical protein |
| | | | MGC16384 (MGC16384), mRNA. |
| 4920110 | GADD45B | 4616 | Homo sapiens growth arrest and DNA-damage-inducible, |
| | | | beta (GADD45B), mRNA. |
| 2260131 | PLCG1 | 5335 | Homo sapiens phospholipase C, gamma 1 (PLCG1), |
| | | | transcript variant 2, mRNA. |
| 3800402 | ESAM | 90952 | Homo sapiens endothelial cell adhesion molecule (ESAM), |
| | | | mRNA. |
| 4560746 | FCAR | 2204 | Homo sapiens Fc fragment of IgA, receptor for (FCAR), |
| | | | transcript variant 3, mRNA. |
| 1690044 | CA5B | 11238 | Homo sapiens carbonic anhydrase VB, mitochondrial |
| | | | (CASE), nuclear gene encoding mitochondrial protein, |
| | | | mRNA. |
| 5340646 | ASAP2 | 8853 | Homo sapiens ArfGAP with SH3 domain, ankyrin repeat |
| | | | and PH domain 2 (ASAP2), transcript variant 1, mRNA. |
| 1440341 | C1QC | 714 | Homo sapiens complement component 1, q subcomponent, |
| | | | C chain (C1QC), mRNA. |
| 1780600 | CD3E | 916 | Homo sapiens CD3e molecule, epsilon (CD3-TCR |
| | | | complex) (CD3E), mRNA. |
| 1190519 | MS4A1 | 931 | Homo sapiens membrane-spanning 4-domains, subfamily |
| | | | A, member 1 (MS4A1), transcript variant 3, mRNA. |
| 270240 | SLC26A8 | 116369 | Homo sapiens solute carrier family 26, member 8 |
| | | | (SLC26A8), transcript variant 1, mRNA. |
| 4290037 | NOV | 4856 | Homo sapiens nephroblastoma overexpressed gene |
| | | | (NOV), mRNA. |
| 7610053 | DDX60 | 55601 | Homo sapiens DEAD (Asp-Glu-Ala-Asp) box polypeptide |
| | | | 60 (DDX60), mRNA. |
| 4860746 | LOC642684 | 642684 | PREDICTED: Homo sapiens similar to Maltase- |
| | | | glucoamylase, intestinal (LOC642684), mRNA. |
| 630397 | KIAA0319L | 79932 | Homo sapiens KIAA0319-like (KIAA0319L), transcript |
| | | | variant 1, mRNA. |
| 5960747 | TRIM22 | 10346 | Homo sapiens tripartite motif-containing 22 (TRIM22), |
| | | | mRNA. |
| 3460014 | C4orf18 | 51313 | Homo sapiens chromosome 4 open reading frame 18 |
| | | | (C4orf18), transcript variant 2, mRNA. |
| 60093 | NCALD | 83988 | Homo sapiens neurocalcin delta (NCALD), mRNA. |
| 4860224 | WARS | 7453 | Homo sapiens tryptophanyl-tRNA synthetase (WARS), |
| | | | transcript variant 1, mRNA. |
| 1940162 | GBP2 | 2634 | Homo sapiens guanylate binding protein 2, interferon- |
| | | | inducible (GBP2), mRNA. |
| 1770243 | IGF2BP3 | 10643 | Homo sapiens insulin-like growth factor 2 mRNA binding |
| | | | protein 3 (IGF2BP3), mRNA. |
| 4540398 | BEND5 | 79656 | Homo sapiens BEN domain containing 5 (BEND5), |
| | | | mRNA. |
| 4220187 | DYSF | 8291 | Homo sapiens dysferlin, limb girdle muscular dystrophy |
| | | | 2B (autosomal recessive) (DYSF), mRNA. |
| 2030360 | FAM159A | 348378 | Homo sapiens family with sequence similarity 159, |
| | | | member A (FAM159A), mRNA. |
| 7330142 | TGFB1I1 | 7041 | Homo sapiens transforming growth factor beta 1 induced |
| | | | transcript 1 (TGFB1I1), transcript variant 2, mRNA. |
| 620750 | CALD1 | 800 | Homo sapiens caldesmon 1 (CALD1), transcript variant 5, |
| | | | mRNA. |
| 5550397 | APOL6 | 80830 | Homo sapiens apolipoprotein L, 6 (APOL6), mRNA. |
| 7040592 | ITGAX | 3687 | PREDICTED: Homo sapiens integrin, alpha X |
| | | | (complement component 3 receptor 4 subunit) (ITGAX), |
| | | | mRNA. |
| 520408 | IFIT3 | 3437 | Homo sapiens interferon-induced protein with |
| | | | tetratricopeptide repeats 3 (IFIT3), mRNA. |
| 4120367 | LPCAT2 | 54947 | Homo sapiens lysophosphatidylcholine acyltransferase 2 |
| | | | (LPCAT2), mRNA. |
| 4070600 | ZNF654 | 55279 | Homo sapiens zinc finger protein 654 (ZNF654), mRNA. |
| 430019 | LOC391045 | 391045 | PREDICTED: Homo sapiens similar to Solute carrier |
| | | | family 2, facilitated glucose transporter member 3 |
| | | | (Glucose transporter type 3, brain) (LOC391045), mRNA. |
| 4850600 | ZNF17 | 7565 | Homo sapiens zinc finger protein 17 (ZNF17), mRNA. |
| 2690670 | ACTR5 | 79913 | Homo sapiens ARP5 actin-related protein 5 homolog |
| | | | (yeast) (ACTR5), mRNA. |
| 2000064 | CTPS2 | 56474 | Homo sapiens CTP synthase II (CTPS2), transcript variant |
| | | | 2, mRNA. |
| 1410221 | S100A12 | 6283 | Homo sapiens S100 calcium binding protein A12 |
| | | | (S100A12), mRNA. |
| 6270553 | CXCL10 | 3627 | Homo sapiens chemokine (C—X—C motif) ligand 10 |
| | | | (CXCL10), mRNA. |
| 3940685 | SIGLEC9 | 27180 | Homo sapiens sialic acid binding Ig-like lectin 9 |
| | | | (SIGLEC9), mRNA. |
| 990014 | C22orf32 | 91689 | Homo sapiens chromosome 22 open reading frame 32 |
| | | | (C22orf32), mRNA. |
| 6370414 | CLECL1 | 160365 | Homo sapiens C-type lectin-like 1 (CLECL1), mRNA. |
| 5890095 | LILRA5 | 353514 | Homo sapiens leukocyte immunoglobulin-like receptor, |
| | | | subfamily A (with TM domain), member 5 (LILRA5), |
| | | | transcript variant 1, mRNA. |
| 1400601 | C20orf100 | 84969 | Homo sapiens chromosome 20 open reading frame 100 |
| | | | (C20orf100), mRNA. |
| 4040187 | C6orf190 | 387357 | Homo sapiens chromosome 6 open reading frame 190 |
| | | | (C6orf190), mRNA. |
| 7650128 | ECGF1 | 1890 | Homo sapiens endothelial cell growth factor 1 (platelet- |
| | | | derived) (ECGF1), mRNA. |
| 6180228 | FCRL3 | 115352 | Homo sapiens Fc receptor-like 3 (FCRL3), transcript |
| | | | variant 2, mRNA. |
| 6350131 | CTRL | 1506 | Homo sapiens chymotrypsin-like (CTRL), mRNA. |
| 5310451 | BCL6 | 604 | Homo sapiens B-cell CLL/lymphoma 6 (zinc finger |
| | | | protein 51) (BCL6), transcript variant 2, mRNA. |
| 7200706 | LOXL3 | 84695 | Homo sapiens lysyl oxidase-like 3 (LOXL3), mRNA. |
| 4150369 | GK | 2710 | Homo sapiens glycerol kinase (GK), transcript variant 1, |
| | | | mRNA. |
| 5340279 | LOC100133565 | 100133565 | PREDICTED: Homo sapiens similar to hCG23738 |
| | | | (LOC100133565), mRNA. |
| 4250136 | LTB4R | 1241 | Homo sapiens leukotriene B4 receptor (LTB4R), mRNA. |
| 6450594 | CD79B | 974 | Homo sapiens CD79B antigen (immunoglobulin- |
| | | | associated beta) (CD79B), transcript variant 1, mRNA. |
| 450202 | ELF2 | 1998 | Homo sapiens E74-like factor 2 (ets domain transcription |
| | | | factor) (ELF2), transcript variant 1, mRNA. |
| 4480615 | PEAR1 | 375033 | Homo sapiens platelet endothelial aggregation receptor 1 |
| | | | (PEAR1), mRNA. |
| 2260725 | MGAM | 8972 | Homo sapiens maltase-glucoamylase (alpha-glucosidase) |
| | | | (MGAM), mRNA. |
| 4670458 | Sep-04 | 5414 | Homo sapiens septin 4 (SEPT4), transcript variant 2, |
| | | | mRNA. |
| 240594 | ANXA3 | 306 | Homo sapiens annexin A3 (ANXA3), mRNA. |
| 6200184 | MEGF9 | 1955 | Homo sapiens multiple EGF-like-domains 9 (MEGF9), |
| | | | mRNA. |
| 2030551 | GPR27 | 2850 | Homo sapiens G protein-coupled receptor 27 (GPR27), |
| | | | mRNA. |
| 4210253 | PTPRJ | 5795 | Homo sapiens protein tyrosine phosphatase, receptor type, |
| | | | J (PTPRJ), mRNA. |
| 4920228 | FFAR2 | 2867 | Homo sapiens free fatty acid receptor 2 (FFAR2), mRNA. |
| 10136 | PML | 5371 | PREDICTED: Homo sapiens promyelocytic leukemia, |
| | | | transcript variant 12 (PML), mRNA. |
| 1430068 | USF1 | 7391 | Homo sapiens upstream transcription factor 1 (USF1), |
| | | | transcript variant 1, mRNA. |
| 3940504 | CD79A | 973 | Homo sapiens CD79a molecule, immunoglobulin- |
| | | | associated alpha (CD79A), transcript variant 2, mRNA. |
| 2810400 | KLHL3 | 26249 | Homo sapiens kelch-like 3 (Drosophila) (KLHL3), |
| | | | mRNA. |
| 1450427 | RTP4 | 64108 | Homo sapiens receptor (chemosensory) transporter protein |
| | | | 4 (RTP4), mRNA. |
| 60554 | PLXNC1 | 10154 | Homo sapiens plexin C1 (PLXNC1), mRNA. |
| 6900528 | RHOT1 | 55288 | Homo sapiens ras homolog gene family, member T1 |
| | | | (RHOT1), transcript variant 1, mRNA. |
| 3610706 | BMX | 660 | Homo sapiens BMX non-receptor tyrosine kinase (BMX), |
| | | | mRNA. |
| 7380626 | FLJ20273 | 54502 | Homo sapiens RNA-binding protein (FLJ20273), mRNA. |
| 7510379 | SACS | 26278 | Homo sapiens spastic ataxia of Charlevoix-Saguenay |
| | | | (sacsin) (SACS), mRNA. |
| 3850441 | MAZ | 4150 | Homo sapiens MYC-associated zinc finger protein |
| | | | (purine-binding transcription factor) (MAZ), transcript |
| | | | variant 2, mRNA. |
| 630017 | ZNF783 | 155060 | Homo sapiens zinc finger family member 783 (ZNF783), |
| | | | mRNA. |
| 7560593 | OSM | 5008 | Homo sapiens oncostatin M (OSM), mRNA. |
| 6560411 | C9orf66 | 157983 | Homo sapiens chromosome 9 open reading frame 66 |
| | | | (C9orf66), mRNA. |
| 20452 | TYMP | 1890 | Homo sapiens thymidine phosphorylase (TYMP), |
| | | | transcript variant 3, mRNA. |
| 4280192 | TARP | 445347 | Homo sapiens TCR gamma alternate reading frame protein |
| | | | (TARP), nuclear gene encoding mitochondrial protein, |
| | | | transcript variant 2, mRNA. |
| 6840767 | FRMD3 | 257019 | Homo sapiens FERM domain containing 3 (FRMD3), |
| | | | mRNA. |
| 6100379 | LOC648984 | 648984 | PREDICTED: Homo sapiens similar to Baculoviral IAP |
| | | | repeat-containing protein 1 (Neuronal apoptosis inhibitory |
| | | | protein) (LOC648984), mRNA. |
| 6220671 | PLAUR | 5329 | Homo sapiens plasminogen activator, urokinase receptor |
| | | | (PLAUR), transcript variant 2, mRNA. |
| 360475 | PLAUR | 5329 | Homo sapiens plasminogen activator, urokinase receptor |
| | | | (PLAUR), transcript variant 2, mRNA. |
| 5420678 | LOC100132119 | 100132119 | PREDICTED: Homo sapiens misc_RNA |
| | | | (LOC100132119), miscRNA. |
| 2970019 | HIST1H4H | 8365 | Homo sapiens histone cluster 1, H4h (HIST1H4H), |
| | | | mRNA. |
| 6370523 | KBTBD8 | 84541 | Homo sapiens kelch repeat and BTB (POZ) domain |
| | | | containing 8 (KBTBD8), mRNA. |
| 1050050 | C9orf45 | 81571 | PREDICTED: Homo sapiens chromosome 9 open reading |
| | | | frame 45 (C9orf45), misc RNA. |
| 3780047 | GBP6 | 163351 | Homo sapiens guanylate binding protein family, member 6 |
| | | | (GBP6), mRNA. |
| 5910064 | TRIM5 | 85363 | Homo sapiens tripartite motif-containing 5 (TRIM5), |
| | | | transcript variant delta, mRNA. |
| 4230102 | SOCS3 | 9021 | Homo sapiens suppressor of cytokine signaling 3 |
| | | | (SOCS3), mRNA. |
| 2030403 | OLIG1 | 116448 | Homo sapiens oligodendrocyte transcription factor 1 |
| | | | (OLIG1), mRNA. |
| 1090739 | LIMS1 | 3987 | Homo sapiens LIM and senescent cell antigen-like |
| | | | domains 1 (LIMS1), mRNA. |
| 6060142 | AFF1 | 4299 | Homo sapiens AF4/FMR2 family, member 1 (AFF1), |
| | | | mRNA. |
| 3060524 | LOC153561 | 153561 | Homo sapiens hypothetical protein LOC153561 |
| | | | (LOC153561), mRNA. |
| 4210544 | SELPLG | 6404 | Homo sapiens selectin P ligand (SELPLG), mRNA. |
| 4810292 | CR2 | 1380 | Homo sapiens complement component (3d/Epstein Barr |
| | | | virus) receptor 2 (CR2), transcript variant 2, mRNA. |
| 2650605 | C4orf18 | 51313 | Homo sapiens chromosome 4 open reading frame 18 |
| | | | (C4orf18), transcript variant 2, mRNA. |
| 7400102 | ASPHD2 | 57168 | Homo sapiens aspartate beta-hydroxylase domain |
| | | | containing 2 (ASPHD2), mRNA. |
| 7160468 | DHRS9 | 10170 | Homo sapiens dehydrogenase/reductase (SDR family) |
| | | | member 9 (DHRS9), transcript variant 1, mRNA. |
| 2120026 | LOC100134300 | 100134300 | PREDICTED: Homo sapiens misc_RNA |
| | | | (LOC100134300), miscRNA. |
| 1500674 | HIST2H3C | 126961 | Homo sapiens histone cluster 2, H3c (HIST2H3C), |
| | | | mRNA. |
| 2140242 | TNFAIP6 | 7130 | Homo sapiens tumor necrosis factor, alpha-induced protein |
| | | | 6 (TNFAIP6), mRNA. |

TABLE 9
List of the 391 genes

| ProbeID | Symbol | Entrez_Gene_ID | Definition |
|---|---|---|---|
| 2190349 | KCNJ15 | 3772 | Homo sapiens potassium inwardly-rectifying channel, |
| | | | subfamily J, member 15 (KCNJ15), transcript variant 3, |
| | | | mRNA. |
| 5860669 | LOC100170939 | 100170939 | Homo sapiens glucuronidase, beta pseudogene |
| | | | (LOC100170939), non-coding RNA. |
| 2100670 | CD177 | 57126 | Homo sapiens CD177 molecule (CD177), mRNA. |
| 4060717 | LOC400499 | 400499 | Homo sapiens hypothetical gene supported by AK126539 |
| | | | (LOC400499), mRNA. |
| 5700725 | EPSTI1 | 94240 | Homo sapiens epithelial stromal interaction 1 (breast) |
| | | | (EPSTI1), transcript variant 2, mRNA. |
| 5220538 | WDFY3 | 23001 | Homo sapiens WD repeat and FYVE domain containing |
| | | | 3 (WDFY3), transcript variant 2, mRNA. |
| 130181 | ANKRD22 | 118932 | Homo sapiens ankyrin repeat domain 22 (ANKRD22), |
| | | | mRNA. |
| 3870138 | TBC1D24 | 57465 | Homo sapiens TBC1 domain family, member 24 |
| | | | (TBC1D24), mRNA. XM_945422 |
| 620136 | LILRA5 | 353514 | Homo sapiens leukocyte immunoglobulin-like receptor, |
| | | | subfamily A (with TM domain), member 5 (LILRA5), |
| | | | transcript variant 3, mRNA. |
| 5860075 | CAMP | 820 | Homo sapiens cathelicidin antimicrobial peptide |
| | | | (CAMP), mRNA. |
| 4290368 | PSTPIP2 | 9050 | Homo sapiens proline-serine-threonine phosphatase |
| | | | interacting protein 2 (PSTPIP2), mRNA. |
| 2630195 | VAMP5 | 10791 | Homo sapiens vesicle-associated membrane protein 5 |
| | | | (myobrevin) (VAMP5), mRNA. |
| 5570398 | FCGR1C | 100132417 | Homo sapiens Fc fragment of IgG, high affinity Ic, |
| | | | receptor (CD64) (FCGR1C), mRNA. |
| 6620209 | FCGR1B | 2210 | Homo sapiens Fc fragment of IgG, high affinity Ib, |
| | | | receptor (CD64) (FCGR1B), transcript variant 2, mRNA. |
| 2230575 | LOC642267 | 642267 | PREDICTED: Homo sapiens similar to hypothetical |
| | | | protein LOC284701, transcript variant 2 (LOC642267), |
| | | | mRNA. |
| 20048 | HIST2H2BF | 440689 | Homo sapiens histone cluster 2, H2bf (HIST2H2BF), |
| | | | mRNA. |
| 3990010 | | | AGENCOURT_7914287 NIH_MGC_71 Homo sapiens |
| | | | cDNA clone IMAGE: 6156595 5, mRNA sequence |
| 1510364 | GBP5 | 115362 | Homo sapiens guanylate binding protein 5 (GBP5), |
| | | | mRNA. |
| 6270681 | BST1 | 683 | Homo sapiens bone marrow stromal cell antigen 1 |
| | | | (BST1), mRNA. |
| 1340241 | C5 | 727 | Homo sapiens complement component 5 (C5), mRNA. |
| 6370768 | ETV7 | 51513 | Homo sapiens ets variant 7 (ETV7), mRNA. |
| 3440095 | PSG3 | 5671 | Homo sapiens pregnancy specific beta-1-glycoprotein 3 |
| | | | (PSG3), mRNA. |
| 50445 | LOC728262 | 728262 | PREDICTED: Homo sapiens hypothetical LOC728262 |
| | | | (LOC728262), mRNA. |
| 1030278 | C10orf33 | 84795 | Homo sapiens chromosome 10 open reading frame 33 |
| | | | (C10orf33), mRNA. |
| 580347 | LOC388572 | 388572 | PREDICTED: Homo sapiens hypothetical LOC388572 |
| | | | (LOC388572), mRNA. |
| 4060014 | LOC441124 | 441124 | PREDICTED: Homo sapiens hypothetical LOC441124 |
| | | | (LOC441124), mRNA. |
| 4280632 | GAS6 | 2621 | Homo sapiens growth arrest-specific 6 (GAS6), mRNA. |
| 770048 | LOC642120 | 642120 | PREDICTED: Homo sapiens similar to Maltase- |
| | | | glucoamylase, intestinal (LOC642120), mRNA. |
| 1470091 | IL15 | 3600 | Homo sapiens interleukin 15 (IL15), transcript variant 1, |
| | | | mRNA. |
| 2490315 | | | Homo sapiens cDNA: FLJ23098 fis, clone LNG07440 |
| 7100161 | VNN2 | 8875 | Homo sapiens vanin 2 (VNN2), transcript variant 1, |
| | | | mRNA. |
| 110639 | MAPK14 | 1432 | Homo sapiens mitogen-activated protein kinase 14 |
| | | | (MAPK14), transcript variant 2, mRNA. |
| 3450349 | CSF2RB | 1439 | Homo sapiens colony stimulating factor 2 receptor, beta, |
| | | | low-affinity (granulocyte-macrophage) (CSF2RB), |
| | | | mRNA. |
| 3180220 | GPR109B | 8843 | Homo sapiens G protein-coupled receptor 109B |
| | | | (GPR109B), mRNA. |
| 1030463 | PROK2 | 60675 | Homo sapiens prokineticin 2 (PROK2), mRNA. |
| 5340324 | FUT7 | 2529 | Homo sapiens fucosyltransferase 7 (alpha (1,3) |
| | | | fucosyltransferase) (FUT7), mRNA. |
| 6840626 | PTGS1 | 5742 | Homo sapiens prostaglandin-endoperoxide synthase 1 |
| | | | (prostaglandin G/H synthase and cyclooxygenase) |
| | | | (PTGS1), transcript variant 1, mRNA. |
| 6560156 | DUSP3 | 1845 | Homo sapiens dual specificity phosphatase 3 (vaccinia |
| | | | virus phosphatase VH1-related) (DUSP3), mRNA. |
| 4200669 | CR1 | 1378 | Homo sapiens complement component (3b/4b) receptor 1 |
| | | | (Knops blood group) (CR1), transcript variant S, mRNA. |
| 3940356 | CR1 | 1378 | Homo sapiens complement component (3b/4b) receptor 1 |
| | | | (Knops blood group) (CR1), transcript variant S, mRNA. |
| 7560039 | TAPBP | 6892 | Homo sapiens TAP binding protein (tapasin) (TAPBP), |
| | | | transcript variant 3, mRNA. |
| 2030309 | SERPING1 | 710 | Homo sapiens serpin peptidase inhibitor, clade G (C1 |
| | | | inhibitor), member 1 (SERPING1), transcript variant 2, |
| | | | mRNA. |
| 5960731 | SOD2 | 6648 | Homo sapiens superoxide dismutase 2, mitochondrial |
| | | | (SOD2), nuclear gene encoding mitochondrial protein, |
| | | | transcript variant 1, mRNA. |
| 4780044 | LOC389386 | 389386 | PREDICTED: Homo sapiens misc_RNA (LOC389386), |
| | | | partial miscRNA. |
| 4810468 | SELP | 6403 | Homo sapiens selectin P (granule membrane protein |
| | | | 140 kDa, antigen CD62) (SELP), mRNA. |
| 5130025 | LOC100134703 | 100134703 | PREDICTED: Homo sapiens misc_RNA |
| | | | (LOC100134703), partial miscRNA. |
| 4120561 | LIMK2 | 3985 | Homo sapiens LIM domain kinase 2 (LIMK2), transcript |
| | | | variant 1, mRNA. |
| 1510255 | LOC648164 | 648164 | PREDICTED: Homo sapiens similar to germinal histone |
| | | | H4 gene, transcript variant 1 (LOC648164), mRNA. |
| 3360491 | MCTP1 | 79772 | Homo sapiens multiple C2 domains, transmembrane 1 |
| | | | (MCTP1), transcript variant L, mRNA. |
| 2230678 | ACACB | 32 | Homo sapiens acetyl-Coenzyme A carboxylase beta |
| | | | (ACACB), mRNA. |
| 730008 | | | xv24e03.x1 Soares_NFL_T_GBC_S1 Homo sapiens |
| | | | cDNA clone IMAGE: 2814076 3, mRNA sequence |
| 5420646 | CETP | 1071 | Homo sapiens cholesteryl ester transfer protein, plasma |
| | | | (CETP), mRNA. |
| 3830228 | GPR109A | 338442 | Homo sapiens G protein-coupled receptor 109A |
| | | | (GPR109A), mRNA. |
| 2510220 | IFI35 | 3430 | Homo sapiens interferon-induced protein 35 (IFI35), |
| | | | mRNA. |
| 1470382 | IRF7 | 3665 | Homo sapiens interferon regulatory factor 7 (IRF7), |
| | | | transcript variant b, mRNA. |
| 3400392 | FPR2 | 2358 | Homo sapiens formyl peptide receptor 2 (FPR2), |
| | | | transcript variant 1, mRNA. |
| 5310445 | KREMEN1 | 83999 | Homo sapiens kringle containing transmembrane protein |
| | | | 1 (KREMEN1), transcript variant 4, mRNA. |
| 3390301 | KREMEN1 | 83999 | Homo sapiens kringle containing transmembrane protein |
| | | | 1 (KREMEN1), transcript variant 4, mRNA. |
| 5310754 | VNN1 | 8876 | Homo sapiens vanin 1 (VNN1), mRNA. |
| 870066 | KLF3 | 51274 | Homo sapiens Kruppel-like factor 3 (basic) (KLF3), |
| | | | mRNA. |
| 6840035 | GBP1 | 2633 | Homo sapiens guanylate binding protein 1, interferon- |
| | | | inducible, 67 kDa (GBP1), mRNA. |
| 2190148 | GBP1 | 2633 | Homo sapiens guanylate binding protein 1, interferon- |
| | | | inducible, 67 kDa (GBP1), mRNA. |
| 7320689 | LOC100133422 | 100133422 | PREDICTED: Homo sapiens hypothetical protein |
| | | | LOC100133422 (LOC100133422), mRNA. |
| 3800398 | FBXO6 | 26270 | Homo sapiens F-box protein 6 (FBXO6), mRNA. |
| 5900524 | LACTB | 114294 | Homo sapiens lactamase, beta (LACTB), nuclear gene |
| | | | encoding mitochondrial protein, transcript variant 2, |
| | | | mRNA. |
| 6220332 | SLC6A12 | 6539 | Homo sapiens solute carrier family 6 (neurotransmitter |
| | | | transporter, betaine/GABA), member 12 (SLC6A12), |
| | | | mRNA. |
| 3840692 | CACNA1S | 779 | Homo sapiens calcium channel, voltage-dependent, L |
| | | | type, alpha 1S subunit (CACNA1S), mRNA. |
| 1400373 | SLA | 6503 | Homo sapiens Src-like-adaptor (SLA), transcript variant |
| | | | 1, mRNA. |
| 3310682 | DOCK5 | 80005 | Homo sapiens dedicator of cytokinesis 5 (DOCK5), |
| | | | mRNA. |
| 6200577 | CREB5 | 9586 | Homo sapiens cAMP responsive element binding protein |
| | | | 5 (CREB5), transcript variant 1, mRNA. |
| 4730059 | BATF2 | 116071 | Homo sapiens basic leucine zipper transcription factor, |
| | | | ATF-like 2 (BATF2), mRNA. |
| 2600735 | TLR6 | 10333 | Homo sapiens toll-like receptor 6 (TLR6), mRNA. |
| 3420241 | SLC2A14 | 144195 | Homo sapiens solute carrier family 2 (facilitated glucose |
| | | | transporter), member 14 (SLC2A14), mRNA. |
| 7050519 | CLEC4D | 338339 | Homo sapiens C-type lectin domain family 4, member D |
| | | | (CLEC4D), mRNA. |
| 4640768 | VPREB3 | 29802 | Homo sapiens pre-B lymphocyte gene 3 (VPREB3), |
| | | | mRNA. |
| 2450392 | LOC642780 | 642780 | PREDICTED: Homo sapiens similar to hypothetical |
| | | | protein LOC284701, transcript variant 2 (LOC642780), |
| | | | mRNA. |
| 6510735 | DDX60L | 91351 | Homo sapiens DEAD (Asp-Glu-Ala-Asp) box |
| | | | polypeptide 60-like (DDX60L), mRNA. |
| 3520044 | LOC100133866 | 100133866 | PREDICTED: Homo sapiens similar to |
| | | | phosphodiesterase 4D interacting protein (myomegalin) |
| | | | (LOC100133866), mRNA. |
| 4150014 | CEACAM1 | 634 | Homo sapiens carcinoembryonic antigen-related cell |
| | | | adhesion molecule 1 (biliary glycoprotein) (CEACAM1), |
| | | | transcript variant 2, mRNA. |
| 5700753 | CEACAM1 | 634 | Homo sapiens carcinoembryonic antigen-related cell |
| | | | adhesion molecule 1 (biliary glycoprotein) (CEACAM1), |
| | | | transcript variant 2, mRNA. |
| 1820725 | LRRK2 | 120892 | Homo sapiens leucine-rich repeat kinase 2 (LRRK2), |
| | | | mRNA. |
| 6200193 | TncRNA | 283131 | PREDICTED: Homo sapiens trophoblast-derived |
| | | | noncoding RNA (TncRNA), misc RNA. |
| 3460053 | SIPA1L2 | 57568 | Homo sapiens signal-induced proliferation-associated 1 |
| | | | like 2 (SIPA1L2), mRNA. |
| 1580435 | TGM2 | 7052 | Homo sapiens transglutaminase 2 (C polypeptide, |
| | | | protein-glutamine-gamma-glutamyltransferase) (TGM2), |
| | | | transcript variant 1, mRNA. |
| 3420593 | LMNB1 | 4001 | Homo sapiens lamin B1 (LMNB1), mRNA. |
| 1500131 | CPEB4 | 80315 | Homo sapiens cytoplasmic polyadenylation element |
| | | | binding protein 4 (CPEB4), mRNA. |
| 1690162 | | | AGENCOURT_14360862 NIH_MGC_187 Homo |
| | | | sapiens cDNA clone IMAGE: 30405414 5, mRNA |
| | | | sequence |
| 6590131 | HP | 3240 | Homo sapiens haptoglobin (HP), mRNA. |
| 3440360 | ZNF438 | 220929 | Homo sapiens zinc finger protein 438 (ZNF438), mRNA. |
| 6220075 | GPR177 | 79971 | Homo sapiens G protein-coupled receptor 177 (GPR177), |
| | | | transcript variant 1, mRNA. |
| 2100458 | LOC729010 | 729010 | PREDICTED: Homo sapiens hypothetical LOC729010 |
| | | | (LOC729010), mRNA. |
| 2810470 | C6orf25 | 80739 | Homo sapiens chromosome 6 open reading frame 25 |
| | | | (C6orf25), transcript variant 4, mRNA. |
| 6100136 | CASP4 | 837 | Homo sapiens caspase 4, apoptosis-related cysteine |
| | | | peptidase (CASP4), transcript variant gamma, mRNA. |
| 6040398 | SLC22A4 | 6583 | Homo sapiens solute carrier family 22 (organic |
| | | | cation/ergothioneine transporter), member 4 (SLC22A4), |
| | | | mRNA. |
| 6420750 | TLR2 | 7097 | Homo sapiens toll-like receptor 2 (TLR2), mRNA. |
| 6590669 | RAB24 | 53917 | Homo sapiens RAB24, member RAS oncogene family |
| | | | (RAB24), transcript variant 1, mRNA. |
| 2750767 | LOC100129960 | 100129960 | PREDICTED: Homo sapiens misc_RNA |
| | | | (LOC100129960), miscRNA. |
| 5270753 | ARG1 | 383 | Homo sapiens arginase, liver (ARG1), mRNA. |
| 3120142 | SULT1A3 | 6818 | Homo sapiens sulfotransferase family, cytosolic, 1A, |
| | | | phenol-preferring, member 3 (SULT1A3), transcript |
| | | | variant 1, mRNA. |
| 540091 | STEAP4 | 79689 | Homo sapiens STEAP family member 4 (STEAP4), |
| | | | mRNA. |
| 3420373 | SOD2 | 6648 | Homo sapiens superoxide dismutase 2, mitochondrial |
| | | | (SOD2), nuclear gene encoding mitochondrial protein, |
| | | | transcript variant 3, mRNA. |
| 6650333 | LOC653867 | 653867 | PREDICTED: Homo sapiens similar to Occludin |
| | | | (LOC653867), mRNA. |
| 2690477 | LOC648710 | 648710 | PREDICTED: Homo sapiens similar to leukocyte |
| | | | immunoglobulin-like receptor, subfamily B (with TM and |
| | | | ITIM domains), member 3 (LOC648710), mRNA. |
| 630255 | LOC652578 | 652578 | PREDICTED: Homo sapiens similar to Fc fragment of |
| | | | IgG, low affinity IIIa, receptor for (CD16) (LOC652578), |
| | | | mRNA. |
| 1190528 | MANSC1 | 54682 | Homo sapiens MANSC domain containing 1 (MANSC1), |
| | | | mRNA. |
| 5890075 | LIN7A | 8825 | Homo sapiens lin-7 homolog A (C. elegans) (LIN7A), |
| | | | mRNA. |
| 3360431 | ZAK | 51776 | Homo sapiens sterile alpha motif and leucine zipper |
| | | | containing kinase AZK (ZAK), transcript variant 1, |
| | | | mRNA. |
| 5390328 | KCNJ15 | 3772 | Homo sapiens potassium inwardly-rectifying channel, |
| | | | subfamily J, member 15 (KCNJ15), transcript variant 1, |
| | | | mRNA. |
| 1050215 | KCNJ15 | 3772 | Homo sapiens potassium inwardly-rectifying channel, |
| | | | subfamily J, member 15 (KCNJ15), transcript variant 1, |
| | | | mRNA. |
| 5290523 | ARHGAP25 | 9938 | Homo sapiens Rho GTPase activating protein 25 |
| | | | (ARHGAP25), transcript variant 1, mRNA. |
| 4570164 | LOC389386 | 389386 | PREDICTED: Homo sapiens misc_RNA (LOC389386), |
| | | | partial miscRNA. |
| 6020097 | SERPINA1 | 5265 | Homo sapiens serpin peptidase inhibitor, clade A (alpha-1 |
| | | | antiproteinase, antitrypsin), member 1 (SERPINA1), |
| | | | transcript variant 2, mRNA. |
| 620403 | LOC400759 | 400759 | Homo sapiens similar to Interferon-induced guanylate- |
| | | | binding protein 1 (GTP-binding protein 1) (Guanine |
| | | | nucleotide-binding protein 1) (HuGBP-1) (LOC400759) |
| | | | on chromosome 1. |
| 6280470 | LOC728519 | 728519 | PREDICTED: Homo sapiens similar to Baculoviral IAP |
| | | | repeat-containing protein 1 (Neuronal apoptosis |
| | | | inhibitory protein) (LOC728519), mRNA. |
| 2630577 | CAV2 | 858 | Homo sapiens caveolin 2 (CAV2), transcript variant 1, |
| | | | mRNA. |
| 5910553 | CAV2 | 858 | Homo sapiens caveolin 2 (CAV2), transcript variant 1, |
| | | | mRNA. |
| 6250010 | GPRIN3 | 285513 | Homo sapiens GPRIN family member 3 (GPRIN3), |
| | | | mRNA. |
| 5570114 | GADD45G | 10912 | Homo sapiens growth arrest and DNA-damage-inducible, |
| | | | gamma (GADD45G), mRNA. |
| 4890181 | RAP1GAP | 5909 | Homo sapiens RAP1 GTPase activating protein |
| | | | (RAP1GAP), mRNA. |
| 1050020 | NLRC4 | 58484 | Homo sapiens NLR family, CARD domain containing 4 |
| | | | (NLRC4), mRNA. |
| 3390121 | CASP5 | 838 | Homo sapiens caspase 5, apoptosis-related cysteine |
| | | | peptidase (CASP5), mRNA. |
| 5960239 | CCR2 | 1231 | Homo sapiens chemokine (C-C motif) receptor 2 (CCR2), |
| | | | transcript variant B, mRNA. |
| 2900524 | CCR2 | 1231 | Homo sapiens chemokine (C-C motif) receptor 2 (CCR2), |
| | | | transcript variant B, mRNA. |
| 4760433 | C16orf7 | 9605 | Homo sapiens chromosome 16 open reading frame 7 |
| | | | (C16orf7), mRNA. |
| 3290458 | FEZ1 | 9638 | Homo sapiens fasciculation and elongation protein zeta 1 |
| | | | (zygin I) (FEZ1), transcript variant 1, mRNA. |
| 2320689 | LOC653610 | 653610 | PREDICTED: Homo sapiens similar to Histone H2A.o |
| | | | (H2A/o) (H2A.2) (H2a-615) (LOC653610), mRNA. |
| 1230047 | CBS | 875 | Homo sapiens cystathionine-beta-synthase (CBS), |
| | | | mRNA. |
| 5130767 | MAPK14 | 1432 | Homo sapiens mitogen-activated protein kinase 14 |
| | | | (MAPK14), transcript variant 1, mRNA. |
| 840685 | IL1B | 3553 | Homo sapiens interleukin 1, beta (IL1B), mRNA. |
| 1260270 | AIM2 | 9447 | Homo sapiens absent in melanoma 2 (AIM2), mRNA. |
| 6200669 | HIST1H2BD | 3017 | Homo sapiens histone cluster 1, H2bd (HIST1H2BD), |
| | | | transcript variant 2, mRNA. |
| 5390427 | NAIP | 4671 | Homo sapiens NLR family, apoptosis inhibitory protein |
| | | | (NAIP), transcript variant 1, mRNA. |
| 5340240 | NAIP | 4671 | Homo sapiens NLR family, apoptosis inhibitory protein |
| | | | (NAIP), transcript variant 1, mRNA. |
| 4860600 | MAPK14 | 1432 | Homo sapiens mitogen-activated protein kinase 14 |
| | | | (MAPK14), transcript variant 3, mRNA. |
| 1010360 | GCH1 | 2643 | Homo sapiens GTP cyclohydrolase 1 (GCH1), transcript |
| | | | variant 3, mRNA. |
| 1170653 | PDGFA | 5154 | Homo sapiens platelet-derived growth factor alpha |
| | | | polypeptide (PDGFA), transcript variant 2, mRNA. |
| 5270377 | LOC728790 | 728790 | PREDICTED: Homo sapiens hypothetical LOC728790 |
| | | | (LOC728790), mRNA. |
| 1510735 | NTN3 | 4917 | Homo sapiens netrin 3 (NTN3), mRNA. |
| 4010270 | LOC440731 | 440731 | PREDICTED: Homo sapiens hypothetical LOC440731, |
| | | | transcript variant 2 (LOC440731), mRNA. |
| 1580360 | LOC651612 | 651612 | PREDICTED: Homo sapiens hypothetical protein |
| | | | LOC651612 (LOC651612), mRNA. |
| 5910019 | C1QB | 713 | Homo sapiens complement component 1, q |
| | | | subcomponent, B chain (C1QB), mRNA. |
| 6620630 | XK | 7504 | Homo sapiens X-linked Kx blood group (McLeod |
| | | | syndrome) (XK), mRNA. |
| 4890707 | PVRL2 | 5819 | Homo sapiens poliovirus receptor-related 2 (herpesvirus |
| | | | entry mediator B) (PVRL2), transcript variant alpha, |
| | | | mRNA. |
| 1010592 | CD36 | 948 | Homo sapiens CD36 molecule (thrombospondin receptor) |
| | | | (CD36), transcript variant 1, mRNA. |
| 2710709 | FCGR1B | 2210 | Homo sapiens Fc fragment of IgG, high affinity Ib, |
| | | | receptor (CD64) (FCGR1B), transcript variant 1, mRNA. |
| 5390747 | SPTB | 6710 | Homo sapiens spectrin, beta, erythrocytic (SPTB), |
| | | | transcript variant 2, mRNA. |
| 3940056 | AIG1 | 51390 | Homo sapiens androgen-induced 1 (AIG1), mRNA. |
| 1190167 | LOC152195 | 152195 | Homo sapiens hypothetical protein LOC152195 |
| | | | (LOC152195), mRNA. |
| 3800050 | ADCY3 | 109 | Homo sapiens adenylate cyclase 3 (ADCY3), mRNA. |
| 830440 | TLR5 | 7100 | Homo sapiens toll-like receptor 5 (TLR5), mRNA. |
| 3140386 | NFATC3 | 4775 | Homo sapiens nuclear factor of activated T-cells, |
| | | | cytoplasmic, calcineurin-dependent 3 (NFATC3), |
| | | | transcript variant 3, mRNA. |
| 3060523 | NAMPT | 10135 | Homo sapiens nicotinamide phosphoribosyltransferase |
| | | | (NAMPT), mRNA. |
| 2230379 | NAMPT | 10135 | Homo sapiens nicotinamide phosphoribosyltransferase |
| | | | (NAMPT), mRNA. |
| 2230402 | TTC35 | 9694 | Homo sapiens tetratricopeptide repeat domain 35 |
| | | | (TTC35), mRNA. |
| 5560270 | CARD17 | 440068 | Homo sapiens caspase recruitment domain family, |
| | | | member 17 (CARD17), mRNA. |
| 3930368 | CARD17 | 440068 | Homo sapiens caspase recruitment domain family, |
| | | | member 17 (CARD17), mRNA. |
| 1740433 | MSL3L1 | 10943 | Homo sapiens male-specific lethal 3-like 1 (Drosophila) |
| | | | (MSL3L1), transcript variant 1, mRNA. |
| 3190053 | LOC642816 | 642816 | PREDICTED: Homo sapiens similar to hypothetical |
| | | | protein LOC284701, transcript variant 1 (LOC642816), |
| | | | mRNA. |
| 360132 | LHFPL2 | 10184 | Homo sapiens lipoma HMGIC fusion partner-like 2 |
| | | | (LHFPL2), mRNA. |
| 1780564 | CDC42 | 998 | Homo sapiens cell division cycle 42 (GTP binding |
| | | | protein, 25 kDa) (CDC42), transcript variant 3, mRNA. |
| 1050025 | CR1 | 1378 | Homo sapiens complement component (3b/4b) receptor 1 |
| | | | (Knops blood group) (CR1), transcript variant F, mRNA. |
| 670167 | TALDO1 | 6888 | PREDICTED: Homo sapiens transaldolase 1 (TALDO1), |
| | | | mRNA. |
| 6420594 | TIFA | 92610 | Homo sapiens TRAF-interacting protein with forkhead- |
| | | | associated domain (TIFA), mRNA. |
| 520523 | CYP4F3 | 4051 | Homo sapiens cytochrome P450, family 4, subfamily F, |
| | | | polypeptide 3 (CYP4F3), mRNA. |
| 1070367 | C19orf59 | 199675 | Homo sapiens chromosome 19 open reading frame 59 |
| | | | (C19orf59), mRNA. |
| 4260017 | CDK5RAP2 | 55755 | Homo sapiens CDK5 regulatory subunit associated |
| | | | protein 2 (CDK5RAP2), transcript variant 2, mRNA. |
| 3890609 | PLSCR1 | 5359 | Homo sapiens phospholipid scramblase 1 (PLSCR1), |
| | | | mRNA. |
| 3780382 | DKFZp761E198 | 91056 | Homo sapiens DKFZp761E198 protein |
| | | | (DKFZp761E198), mRNA. |
| 5560280 | SAMD14 | 201191 | Homo sapiens sterile alpha motif domain containing 14 |
| | | | (SAMD14), mRNA. |
| 3180209 | ANK1 | 286 | Homo sapiens ankyrin 1, erythrocytic (ANK1), transcript |
| | | | variant 5, mRNA. |
| 5700735 | PARP9 | 83666 | Homo sapiens poly (ADP-ribose) polymerase family, |
| | | | member 9 (PARP9), mRNA. |
| 1940132 | CD151 | 977 | Homo sapiens CD151 molecule (Raph blood group) |
| | | | (CD151), transcript variant 4, mRNA. |
| 6650064 | SIGLEC5 | 8778 | Homo sapiens sialic acid binding Ig-like lectin 5 |
| | | | (SIGLEC5), mRNA. |
| 4640270 | SIGLEC5 | 8778 | Homo sapiens sialic acid binding Ig-like lectin 5 |
| | | | (SIGLEC5), mRNA. |
| 1030431 | ACSL1 | 2180 | Homo sapiens acyl-CoA synthetase long-chain family |
| | | | member 1 (ACSL1), mRNA. |
| 3360132 | SNX20 | 124460 | Homo sapiens sorting nexin 20 (SNX20), mRNA. |
| 5810047 | F2RL1 | 2150 | Homo sapiens coagulation factor II (thrombin) receptor- |
| | | | like 1 (F2RL1), mRNA. |
| 2470601 | IL1RN | 3557 | Homo sapiens interleukin 1 receptor antagonist (IL1RN), |
| | | | transcript variant 1, mRNA. |
| 60719 | KCNJ2 | 3759 | Homo sapiens potassium inwardly-rectifying channel, |
| | | | subfamily J, member 2 (KCNJ2), mRNA. |
| 5340767 | CEACAM1 | 634 | Homo sapiens carcinoembryonic antigen-related cell |
| | | | adhesion molecule 1 (biliary glycoprotein) (CEACAM1), |
| | | | transcript variant 1, mRNA. |
| 4250678 | MGC16121 | 84848 | PREDICTED: Homo sapiens hypothetical protein |
| | | | MGC16121 (MGC16121), mRNA. |
| 7610440 | XAF1 | 54739 | Homo sapiens XIAP associated factor 1 (XAF1), |
| | | | transcript variant 2, mRNA. |
| 2810692 | KIAA1881 | 114782 | PREDICTED: Homo sapiens KIAA1881 (KIAA1881), |
| | | | mRNA. |
| 2710646 | MSRB2 | 22921 | Homo sapiens methionine sulfoxide reductase B2 |
| | | | (MSRB2), mRNA. |
| 3310341 | FYB | 2533 | Homo sapiens FYN binding protein (FYB-120/130) |
| | | | (FYB), transcript variant 1, mRNA. |
| 990470 | LOC651738 | 651738 | PREDICTED: Homo sapiens hypothetical LOC651738 |
| | | | (LOC651738), mRNA. |
| 3310376 | TIMM10 | 26519 | Homo sapiens translocase of inner mitochondrial |
| | | | membrane 10 homolog (yeast) (TIMM10), nuclear gene |
| | | | encoding mitochondrial protein, mRNA. |
| 6510707 | FER1L3 | 26509 | Homo sapiens fer-1-like 3, myoferlin (C. elegans) |
| | | | (FER1L3), transcript variant 1, mRNA. |
| 1400593 | SIGLEC14 | 100049587 | Homo sapiens sialic acid binding Ig-like lectin 14 |
| | | | (SIGLEC14), mRNA. |
| 4060088 | LOC644677 | 644677 | PREDICTED: Homo sapiens hypothetical protein |
| | | | LOC644677 (LOC644677), mRNA. |
| 3800019 | NCR3 | 259197 | Homo sapiens natural cytotoxicity triggering receptor 3 |
| | | | (NCR3), mRNA. |
| 3830433 | | | BX096301 Soares retina N2b5HR Homo sapiens cDNA |
| | | | clone IMAGp998C16444, mRNA sequence |
| 2690435 | IFIT1 | 3434 | Homo sapiens interferon-induced protein with |
| | | | tetratricopeptide repeats 1 (IFIT1), transcript variant 2, |
| | | | mRNA. |
| 1710553 | HSPA6 | 3310 | Homo sapiens heat shock 70 kDa protein 6 (HSP70B′) |
| | | | (HSPA6), mRNA. |
| 2030678 | HIST2H2AB | 317772 | Homo sapiens histone cluster 2, H2ab (HIST2H2AB), |
| | | | mRNA. |
| 1570048 | PIK3R5 | 23533 | Homo sapiens phosphoinositide-3-kinase, regulatory |
| | | | subunit 5 (PIK3R5), mRNA. |
| 4290619 | CREB5 | 9586 | Homo sapiens cAMP responsive element binding protein |
| | | | 5 (CREB5), transcript variant 4, mRNA. |
| 2640603 | CLEC7A | 64581 | Homo sapiens C-type lectin domain family 7, member A |
| | | | (CLEC7A), transcript variant 5, mRNA. |
| 3360343 | RSAD2 | 91543 | Homo sapiens radical S-adenosyl methionine domain |
| | | | containing 2 (RSAD2), mRNA. |
| 7160066 | C20orf196 | 149840 | Homo sapiens chromosome 20 open reading frame 196 |
| | | | (C20orf196), mRNA. |
| 2480327 | MYL9 | 10398 | Homo sapiens myosin, light chain 9, regulatory (MYL9), |
| | | | transcript variant 1, mRNA. |
| 1580750 | LOC100130828 | 100130828 | PREDICTED: Homo sapiens hypothetical |
| | | | LOC100130828 (LOC100130828), mRNA. |
| 1010246 | IFI6 | 2537 | Homo sapiens interferon, alpha-inducible protein 6 |
| | | | (IFI6), transcript variant 2, mRNA. |
| 7040035 | OAS1 | 4938 | Homo sapiens 2′,5′-oligoadenylate synthetase 1, |
| | | | 40/46 kDa (OAS1), transcript variant 3, mRNA. |
| 2140020 | LIMK2 | 3985 | Homo sapiens LIM domain kinase 2 (LIMK2), transcript |
| | | | variant 2b, mRNA. |
| 3390048 | LIMK2 | 3985 | Homo sapiens LIM domain kinase 2 (LIMK2), transcript |
| | | | variant 2b, mRNA. |
| 5910632 | SMARCD3 | 6604 | Homo sapiens SWI/SNF related, matrix associated, actin |
| | | | dependent regulator of chromatin, subfamily d, member 3 |
| | | | (SMARCD3), transcript variant 2, mRNA. |
| 5050537 | OPLAH | 26873 | Homo sapiens 5-oxoprolinase (ATP-hydrolysing) |
| | | | (OPLAH), mRNA. |
| 5550634 | BEX1 | 55859 | Homo sapiens brain expressed, X-linked 1 (BEX1), |
| | | | mRNA. |
| 4900747 | C11orf75 | 56935 | Homo sapiens chromosome 11 open reading frame 75 |
| | | | (C11orf75), mRNA. |
| 1240358 | RAB20 | 55647 | Homo sapiens RAB20, member RAS oncogene family |
| | | | (RAB20), mRNA. |
| 7040477 | PLOD2 | 5352 | Homo sapiens procollagen-lysine, 2-oxoglutarate 5- |
| | | | dioxygenase 2 (PLOD2), transcript variant 2, mRNA. |
| 7200338 | BMP2K | 55589 | Homo sapiens BMP2 inducible kinase (BMP2K), |
| | | | transcript variant 1, mRNA. |
| 1470685 | ITGB3 | 3690 | Homo sapiens integrin, beta 3 (platelet glycoprotein IIIa, |
| | | | antigen CD61) (ITGB3), mRNA. |
| 630315 | DHRS9 | 10170 | Homo sapiens dehydrogenase/reductase (SDR family) |
| | | | member 9 (DHRS9), transcript variant 1, mRNA. |
| 730561 | ARHGEF10L | 55160 | Homo sapiens Rho guanine nucleotide exchange factor |
| | | | (GEF) 10-like (ARHGEF10L), transcript variant 1, |
| | | | mRNA. |
| 6760286 | | | RST24587 Athersys RAGE Library Homo sapiens |
| | | | cDNA, mRNA sequence |
| 3130220 | TMEM158 | 25907 | Homo sapiens transmembrane protein 158 (TMEM158), |
| | | | mRNA. |
| 3290292 | LAP3 | 51056 | Homo sapiens leucine aminopeptidase 3 (LAP3), mRNA. |
| 3780187 | GYPB | 2994 | Homo sapiens glycophorin B (MNS blood group) |
| | | | (GYPB), mRNA. |
| 1090538 | LOC100128737 | 100128737 | PREDICTED: Homo sapiens hypothetical protein |
| | | | LOC100128737 (LOC100128737), mRNA. |
| 4850592 | P2RY14 | 9934 | Homo sapiens purinergic receptor P2Y, G-protein |
| | | | coupled, 14 (P2RY14), transcript variant 2, mRNA. |
| 4610241 | P2RY14 | 9934 | Homo sapiens purinergic receptor P2Y, G-protein |
| | | | coupled, 14 (P2RY14), transcript variant 2, mRNA. |
| 6510170 | IFIT3 | 3437 | Homo sapiens interferon-induced protein with |
| | | | tetratricopeptide repeats 3 (IFIT3), mRNA. |
| 1500280 | IFIT3 | 3437 | Homo sapiens interferon-induced protein with |
| | | | tetratricopeptide repeats 3 (IFIT3), mRNA. |
| 4180544 | ROPN1L | 83853 | Homo sapiens ropporin 1-like (ROPN1L), mRNA. |
| 1440360 | GPR141 | 353345 | Homo sapiens G protein-coupled receptor 141 (GPR141), |
| | | | mRNA. |
| 4150270 | ANKRD22 | 118932 | Homo sapiens ankyrin repeat domain 22 (ANKRD22), |
| | | | mRNA. |
| 4230328 | CALD1 | 800 | Homo sapiens caldesmon 1 (CALD1), transcript variant |
| | | | 3, mRNA. |
| 3990673 | AFF1 | 4299 | Homo sapiens AF4/FMR2 family, member 1 (AFF1), |
| | | | mRNA. |
| 2140524 | HIST1H3D | 8351 | Homo sapiens histone cluster 1, H3d (HIST1H3D), |
| | | | mRNA. |
| 7610113 | SLC26A8 | 116369 | Homo sapiens solute carrier family 26, member 8 |
| | | | (SLC26A8), transcript variant 2, mRNA. |
| 5360626 | SLC26A8 | 116369 | Homo sapiens solute carrier family 26, member 8 |
| | | | (SLC26A8), transcript variant 2, mRNA. |
| 7000368 | UBE2L6 | 9246 | Homo sapiens ubiquitin-conjugating enzyme E2L 6 |
| | | | (UBE2L6), transcript variant 1, mRNA. |
| 770088 | DTX3L | 151636 | Homo sapiens deltex 3-like (Drosophila) (DTX3L), |
| | | | mRNA. |
| 1980431 | DOK3 | 79930 | Homo sapiens docking protein 3 (DOK3), mRNA. |
| 4780068 | SULT1B1 | 27284 | Homo sapiens sulfotransferase family, cytosolic, 1B, |
| | | | member 1 (SULT1B1), mRNA. |
| 5670465 | ADM | 133 | Homo sapiens adrenomedullin (ADM), mRNA. |
| 6420646 | BZRAP1 | 9256 | Homo sapiens benzodiazapine receptor (peripheral) |
| | | | associated protein 1 (BZRAP1), mRNA. |
| 510014 | | | UI-H-BW1-anh-g-12-0-UI.s1 NCI_CGAP_Sub7 Homo |
| | | | sapiens cDNA clone IMAGE: 3082558 3, mRNA |
| | | | sequence |
| 620091 | LOC730234 | 730234 | PREDICTED: Homo sapiens misc_RNA (LOC730234), |
| | | | miscRNA. |
| 610451 | HIST2H2AA3 | 8337 | Homo sapiens histone cluster 2, H2aa3 (HIST2H2AA3), |
| | | | mRNA. |
| 5220142 | LIMK2 | 3985 | Homo sapiens LIM domain kinase 2 (LIMK2), transcript |
| | | | variant 2a, mRNA. |
| 4730746 | PADI2 | 11240 | Homo sapiens peptidyl arginine deiminase, type II |
| | | | (PADI2), mRNA. |
| 110397 | LOC652755 | 652755 | PREDICTED: Homo sapiens similar to Baculoviral IAP |
| | | | repeat-containing protein 1 (Neuronal apoptosis |
| | | | inhibitory protein) (LOC652755), mRNA. |
| 2570041 | WASF3 | 10810 | Homo sapiens WAS protein family, member 3 (WASF3), |
| | | | mRNA. |
| 450348 | GNG10 | 2790 | Homo sapiens guanine nucleotide binding protein (G |
| | | | protein), gamma 10 (GNG10), mRNA. |
| 4570725 | LOC653604 | 653604 | PREDICTED: Homo sapiens similar to H3 histone, |
| | | | family 2 isoform 2 (LOC653604), mRNA. |
| 3130246 | RNF149 | 284996 | Homo sapiens ring finger protein 149 (RNF149), mRNA. |
| 5340692 | STRBP | 55342 | Homo sapiens spermatid perinuclear RNA binding |
| | | | protein (STRBP), mRNA. |
| 4200754 | ST3GAL4 | 6484 | Homo sapiens ST3 beta-galactoside alpha-2,3- |
| | | | sialyltransferase 4 (ST3GAL4), mRNA. |
| 6980133 | | | HUMGS0004661 Human adult (K. Okubo) Homo sapiens |
| | | | cDNA 3, mRNA sequence |
| 6590646 | FAM26F | 441168 | Homo sapiens family with sequence similarity 26, |
| | | | member F (FAM26F), mRNA. |
| 4810139 | C9orf40 | 55071 | Homo sapiens chromosome 9 open reading frame 40 |
| | | | (C9orf40), mRNA. |
| 270100 | EYS | 346007 | Homo sapiens eyes shut homolog (Drosophila) (EYS), |
| | | | transcript variant 3, mRNA. |
| 3850661 | C3orf23 | 285343 | Homo sapiens chromosome 3 open reading frame 23 |
| | | | (C3orf23), transcript variant 3, mRNA. |
| 3370400 | LOC643313 | 643313 | PREDICTED: Homo sapiens similar to hypothetical |
| | | | protein LOC284701, transcript variant 1 (LOC643313), |
| | | | mRNA. |
| 4900239 | CD274 | 29126 | Homo sapiens CD274 molecule (CD274), mRNA. |
| 940274 | GK | 2710 | Homo sapiens glycerol kinase (GK), transcript variant 2, |
| | | | mRNA. |
| 6380672 | CA4 | 762 | Homo sapiens carbonic anhydrase IV (CA4), mRNA. |
| 6650242 | IFITM3 | 10410 | Homo sapiens interferon induced transmembrane protein |
| | | | 3 (1-8U) (IFITM3), mRNA. |
| 2810450 | MME | 4311 | Homo sapiens membrane metallo-endopeptidase (MME), |
| | | | transcript variant 1, mRNA. |
| 1820750 | STAT1 | 6772 | Homo sapiens signal transducer and activator of |
| | | | transcription 1, 91 kDa (STAT1), transcript variant alpha, |
| | | | mRNA. |
| 5890270 | LOC441151 | 441151 | PREDICTED: Homo sapiens similar to expressed |
| | | | sequence AW125688 (LOC441151), mRNA. |
| 1260066 | | | Homo sapiens cDNA: FLJ21199 fis, clone COL00235 |
| 5550553 | DHRS13 | 147015 | Homo sapiens dehydrogenase/reductase (SDR family) |
| | | | member 13 (DHRS13), mRNA. |
| 5870608 | PPP1R3B | 79660 | Homo sapiens protein phosphatase 1, regulatory |
| | | | (inhibitor) subunit 3B (PPP1R3B), mRNA. |
| 3870338 | IFI44L | 10964 | Homo sapiens interferon-induced protein 44-like |
| | | | (IFI44L), mRNA. |
| 6660162 | LRG1 | 116844 | Homo sapiens leucine-rich alpha-2-glycoprotein 1 |
| | | | (LRG1), mRNA. |
| 990349 | MCTP2 | 55784 | Homo sapiens multiple C2 domains, transmembrane 2 |
| | | | (MCTP2), mRNA. |
| 7400372 | WSB1 | 26118 | Homo sapiens WD repeat and SOCS box-containing 1 |
| | | | (WSB1), transcript variant 3, mRNA. |
| 3800241 | CDH6 | 1004 | Homo sapiens cadherin 6, type 2, K-cadherin (fetal |
| | | | kidney) (CDH6), mRNA. |
| 1500327 | C4orf23 | 152992 | Homo sapiens chromosome 4 open reading frame 23 |
| | | | (C4orf23), transcript variant 2, mRNA. |
| 5550474 | RAB24 | 53917 | Homo sapiens RAB24, member RAS oncogene family |
| | | | (RAB24), transcript variant 2, mRNA. |
| 830152 | FCER2 | 2208 | Homo sapiens Fc fragment of IgE, low affinity II, |
| | | | receptor for (CD23) (FCER2), mRNA. |
| 7510537 | SCO2 | 9997 | Homo sapiens SCO cytochrome oxidase deficient |
| | | | homolog 2 (yeast) (SCO2), nuclear gene encoding |
| | | | mitochondrial protein, mRNA. |
| 940519 | GPR44 | 11251 | Homo sapiens G protein-coupled receptor 44 (GPR44), |
| | | | mRNA. |
| 1410192 | TDRD9 | 122402 | Homo sapiens tudor domain containing 9 (TDRD9), |
| | | | mRNA. |
| 6180187 | TDRD9 | 122402 | Homo sapiens tudor domain containing 9 (TDRD9), |
| | | | mRNA. |
| 380082 | LOC646144 | 646144 | PREDICTED: Homo sapiens similar to tousled-like |
| | | | kinase 2, transcript variant 2 (LOC646144), mRNA. |
| 1470470 | GPR97 | 222487 | Homo sapiens G protein-coupled receptor 97 (GPR97), |
| | | | mRNA. |
| 5260424 | GPR97 | 222487 | Homo sapiens G protein-coupled receptor 97 (GPR97), |
| | | | mRNA. |
| 4230441 | GPR97 | 222487 | Homo sapiens G protein-coupled receptor 97 (GPR97), |
| | | | mRNA. |
| 2370743 | LOC100132858 | 100132858 | PREDICTED: Homo sapiens hypothetical protein |
| | | | LOC100132858 (LOC100132858), mRNA. |
| 6520180 | IL18RAP | 8807 | Homo sapiens interleukin 18 receptor accessory protein |
| | | | (IL18RAP), mRNA. |
| 4890598 | BRSK1 | 84446 | Homo sapiens BR serine/threonine kinase 1 (BRSK1), |
| | | | mRNA. |
| 6760593 | OSBPL10 | 114884 | Homo sapiens oxysterol binding protein-like 10 |
| | | | (OSBPL10), mRNA. |
| 3140487 | BMX | 660 | Homo sapiens BMX non-receptor tyrosine kinase (BMX), |
| | | | mRNA. |
| 520086 | FCGR1A | 2209 | Homo sapiens Fc fragment of IgG, high affinity Ia, |
| | | | receptor (CD64) (FCGR1A), mRNA. |
| 2570300 | IFI44 | 10561 | Homo sapiens interferon-induced protein 44 (IFI44), |
| | | | mRNA. |
| 1500180 | TLR4 | 7099 | Homo sapiens toll-like receptor 4 (TLR4), mRNA. |
| 2710400 | DSC2 | 1824 | Homo sapiens desmocollin 2 (DSC2), transcript variant |
| | | | Dsc2b, mRNA. |
| 2000068 | PICALM | 8301 | Homo sapiens phosphatidylinositol binding clathrin |
| | | | assembly protein (PICALM), transcript variant 1, mRNA. |
| 630619 | HPSE | 10855 | Homo sapiens heparanase (HPSE), mRNA. |
| 6290672 | LOC728417 | 728417 | PREDICTED: Homo sapiens hypothetical protein |
| | | | LOC728417 (LOC728417), mRNA. |
| 2570482 | LOC728417 | 728417 | PREDICTED: Homo sapiens hypothetical protein |
| | | | LOC728417 (LOC728417), mRNA. |
| 5560471 | FCAR | 2204 | Homo sapiens Fc fragment of IgA, receptor for (FCAR), |
| | | | transcript variant 10, mRNA. |
| 3420259 | MIR21 | 406991 | Homo sapiens microRNA 21 (MIR21), microRNA. |
| 5340270 | LOC100134083 | 100134083 | PREDICTED: Homo sapiens similar to hCG1647242 |
| | | | (LOC100134083), mRNA. |
| 2030170 | CARD16 | 114769 | Homo sapiens caspase recruitment domain family, |
| | | | member 16 (CARD16), transcript variant 2, mRNA. |
| 5270725 | CARD16 | 114769 | Homo sapiens caspase recruitment domain family, |
| | | | member 16 (CARD16), transcript variant 2, mRNA. |
| 870193 | SP140 | 11262 | Homo sapiens SP140 nuclear body protein (SP140), |
| | | | transcript variant 2, mRNA. |
| 5570039 | LOC728744 | 728744 | PREDICTED: Homo sapiens hypothetical LOC728744 |
| | | | (LOC728744), mRNA. |
| 3360372 | CCRL2 | 9034 | Homo sapiens chemokine (C-C motif) receptor-like 2 |
| | | | (CCRL2), transcript variant 2, mRNA. |
| 3990170 | IFI27 | 3429 | Homo sapiens interferon, alpha-inducible protein 27 |
| | | | (IFI27), transcript variant 2, mRNA. |
| 7040181 | PFKFB2 | 5208 | Homo sapiens 6-phosphofructo-2-kinase/fructose-2,6- |
| | | | biphosphatase 2 (PFKFB2), transcript variant 2, mRNA. |
| 5900543 | Sep-04 | 5414 | Homo sapiens septin 4 (SEPT4), transcript variant 3, |
| | | | mRNA. |
| 3180528 | MMP9 | 4318 | Homo sapiens matrix metallopeptidase 9 (gelatinase B, |
| | | | 92 kDa gelatinase, 92 kDa type IV collagenase) (MMP9), |
| | | | mRNA. |
| 460463 | SMARCD3 | 6604 | Homo sapiens SWI/SNF related, matrix associated, actin |
| | | | dependent regulator of chromatin, subfamily d, member 3 |
| | | | (SMARCD3), transcript variant 1, mRNA. |
| 4260204 | HIP1R | 9026 | Homo sapiens huntingtin interacting protein 1 related |
| | | | (HIP1R), mRNA. |
| 5870678 | LOC441763 | 441763 | PREDICTED: Homo sapiens hypothetical LOC441763 |
| | | | (LOC441763), mRNA. |
| 110148 | BCL10 | 8915 | Homo sapiens B-cell CLL/lymphoma 10 (BCL10), |
| | | | mRNA. |
| 4860753 | ASGR2 | 433 | Homo sapiens asialoglycoprotein receptor 2 (ASGR2), |
| | | | transcript variant 3, mRNA. |
| 3800576 | ASGR2 | 433 | Homo sapiens asialoglycoprotein receptor 2 (ASGR2), |
| | | | transcript variant 3, mRNA. |
| 2680598 | TMEM150B | 284417 | Homo sapiens transmembrane protein 150B |
| | | | (TMEM150B), mRNA. |
| 5270674 | GPR68 | 8111 | Homo sapiens G protein-coupled receptor 68 (GPR68), |
| | | | mRNA. |
| 5810689 | PRRG4 | 79056 | Homo sapiens proline rich Gla (G-carboxyglutamic acid) |
| | | | 4 (transmembrane) (PRRG4), mRNA. |
| 4210722 | LOC641693 | 641693 | PREDICTED: Homo sapiens hypothetical protein |
| | | | LOC641693 (LOC641693), mRNA. |
| 3870170 | LOC728093 | 728093 | PREDICTED: Homo sapiens misc_RNA (LOC728093), |
| | | | partial miscRNA. |
| 7100703 | LOC100132287 | 100132287 | PREDICTED: Homo sapiens misc_RNA |
| | | | (LOC100132287), miscRNA. |
| 6220739 | GRAMD1B | 57476 | Homo sapiens GRAM domain containing 1B |
| | | | (GRAMD1B), mRNA. |
| 1030376 | PLCG1 | 5335 | Homo sapiens phospholipase C, gamma 1 (PLCG1), |
| | | | transcript variant 2, mRNA. |
| 4560746 | FCAR | 2204 | Homo sapiens Fc fragment of IgA, receptor for (FCAR), |
| | | | transcript variant 3, mRNA. |
| 3440369 | CD84 | 8832 | Homo sapiens CD84 molecule (CD84), mRNA. |
| 1440341 | C1QC | 714 | Homo sapiens complement component 1, q |
| | | | subcomponent, C chain (C1QC), mRNA. |
| 270240 | SLC26A8 | 116369 | Homo sapiens solute carrier family 26, member 8 |
| | | | (SLC26A8), transcript variant 1, mRNA. |
| 4860746 | LOC642684 | 642684 | PREDICTED: Homo sapiens similar to Maltase- |
| | | | glucoamylase, intestinal (LOC642684), mRNA. |
| 610270 | ELL2 | 22936 | Homo sapiens elongation factor, RNA polymerase II, 2 |
| | | | (ELL2), mRNA. |
| 830142 | PHC2 | 1912 | Homo sapiens polyhomeotic-like 2 (Drosophila) (PHC2), |
| | | | transcript variant 2, mRNA. |
| 4880615 | LOC646309 | 646309 | PREDICTED: Homo sapiens similar to Nicotinamide |
| | | | phosphoribosyltransferase (NAmPRTase) (Nampt) (Pre-B |
| | | | cell enhancing factor) (Pre-B-cell colony-enhancing |
| | | | factor 1) (LOC646309), mRNA. |
| 1770243 | IGF2BP3 | 10643 | Homo sapiens insulin-like growth factor 2 mRNA |
| | | | binding protein 3 (IGF2BP3), mRNA. |
| 4220187 | DYSF | 8291 | Homo sapiens dysferlin, limb girdle muscular dystrophy |
| | | | 2B (autosomal recessive) (DYSF), mRNA. |
| 6180427 | G0S2 | 50486 | Homo sapiens G0/G1switch 2 (G0S2), mRNA. |
| 7330142 | TGFB1I1 | 7041 | Homo sapiens transforming growth factor beta 1 induced |
| | | | transcript 1 (TGFB1I1), transcript variant 2, mRNA. |
| 620750 | CALD1 | 800 | Homo sapiens caldesmon 1 (CALD1), transcript variant |
| | | | 5, mRNA. |
| 540161 | DNAJC25- | 552891 | Homo sapiens DNAJC25-GNG10 readthrough |
| | GNG10 | | (DNAJC25-GNG10), mRNA. |
| 5310377 | DSC2 | 1824 | Homo sapiens desmocollin 2 (DSC2), transcript variant |
| | | | Dsc2a, mRNA. |
| 5550397 | APOL6 | 80830 | Homo sapiens apolipoprotein L, 6 (APOL6), mRNA. |
| 7040592 | ITGAX | 3687 | PREDICTED: Homo sapiens integrin, alpha X |
| | | | (complement component 3 receptor 4 subunit) (ITGAX), |
| | | | mRNA. |
| 520408 | IFIT3 | 3437 | Homo sapiens interferon-induced protein with |
| | | | tetratricopeptide repeats 3 (IFIT3), mRNA. |
| 4120367 | LPCAT2 | 54947 | Homo sapiens lysophosphatidylcholine acyltransferase 2 |
| | | | (LPCAT2), mRNA. |
| 430019 | LOC391045 | 391045 | PREDICTED: Homo sapiens similar to Solute carrier |
| | | | family 2, facilitated glucose transporter member 3 |
| | | | (Glucose transporter type 3, brain) (LOC391045), |
| | | | mRNA. |
| 1230671 | LOC100134822 | 100134822 | PREDICTED: Homo sapiens misc_RNA |
| | | | (LOC100134822), miscRNA. |
| 1410221 | S100A12 | 6283 | Homo sapiens S100 calcium binding protein A12 |
| | | | (S100A12), mRNA. |
| 6270553 | CXCL10 | 3627 | Homo sapiens chemokine (C—X—C motif) ligand 10 |
| | | | (CXCL10), mRNA. |
| 6370414 | CLECL1 | 160365 | Homo sapiens C-type lectin-like 1 (CLECL1), mRNA. |
| 5890095 | LILRA5 | 353514 | Homo sapiens leukocyte immunoglobulin-like receptor, |
| | | | subfamily A (with TM domain), member 5 (LILRA5), |
| | | | transcript variant 1, mRNA. |
| 5310451 | BCL6 | 604 | Homo sapiens B-cell CLL/lymphoma 6 (zinc finger |
| | | | protein 51) (BCL6), transcript variant 2, mRNA. |
| 4150369 | GK | 2710 | Homo sapiens glycerol kinase (GK), transcript variant 1, |
| | | | mRNA. |
| 6370053 | RTBDN | 83546 | Homo sapiens retbindin (RTBDN), transcript variant 2, |
| | | | mRNA. |
| 5340279 | LOC100133565 | 100133565 | PREDICTED: Homo sapiens similar to hCG23738 |
| | | | (LOC100133565), mRNA. |
| 2480072 | SLC22A16 | 85413 | Homo sapiens solute carrier family 22 (organic |
| | | | cation/carnitine transporter), member 16 (SLC22A16), |
| | | | mRNA. |
| 5390093 | INSL3 | 3640 | Homo sapiens insulin-like 3 (Leydig cell) (INSL3), |
| | | | mRNA. |
| 450202 | ELF2 | 1998 | Homo sapiens E74-like factor 2 (ets domain transcription |
| | | | factor) (ELF2), transcript variant 1, mRNA. |
| 4670458 | Sep-04 | 5414 | Homo sapiens septin 4 (SEPT4), transcript variant 2, |
| | | | mRNA. |
| 240594 | ANXA3 | 306 | Homo sapiens annexin A3 (ANXA3), mRNA. |
| 6200184 | MEGF9 | 1955 | Homo sapiens multiple EGF-like-domains 9 (MEGF9), |
| | | | mRNA. |
| 2030551 | GPR27 | 2850 | Homo sapiens G protein-coupled receptor 27 (GPR27), |
| | | | mRNA. |
| 4920228 | FFAR2 | 2867 | Homo sapiens free fatty acid receptor 2 (FFAR2), |
| | | | mRNA. |
| 1430068 | USF1 | 7391 | Homo sapiens upstream transcription factor 1 (USF1), |
| | | | transcript variant 1, mRNA. |
| 3060435 | LOC652750 | 652750 | PREDICTED: Homo sapiens misc_RNA (LOC652750), |
| | | | partial miscRNA. |
| 3450521 | ECM1 | 1893 | Homo sapiens extracellular matrix protein 1 (ECM1), |
| | | | transcript variant 2, mRNA. |
| 1450427 | RTP4 | 64108 | Homo sapiens receptor (chemosensory) transporter |
| | | | protein 4 (RTP4), mRNA. |
| 6900528 | RHOT1 | 55288 | Homo sapiens ras homolog gene family, member T1 |
| | | | (RHOT1), transcript variant 1, mRNA. |
| 3610706 | BMX | 660 | Homo sapiens BMX non-receptor tyrosine kinase (BMX), |
| | | | mRNA. |
| 4610538 | SUMO1P1 | 391257 | Homo sapiens SUMO1 pseudogene 1 (SUMO1P1) on |
| | | | chromosome 20. |
| 7510379 | SACS | 26278 | Homo sapiens spastic ataxia of Charlevoix-Saguenay |
| | | | (sacsin) (SACS), mRNA. |
| 3990411 | C9orf72 | 203228 | Homo sapiens chromosome 9 open reading frame 72 |
| | | | (C9orf72), transcript variant 1, mRNA. |
| 3850441 | MAZ | 4150 | Homo sapiens MYC-associated zinc finger protein |
| | | | (purine-binding transcription factor) (MAZ), transcript |
| | | | variant 2, mRNA. |
| 3710253 | CAPN12 | 147968 | Homo sapiens calpain 12 (CAPN12), mRNA. |
| 840433 | RSPH9 | 221421 | Homo sapiens radial spoke head 9 homolog |
| | | | (Chlamydomonas) (RSPH9), mRNA. |
| 360360 | LOC100129343 | 100129343 | PREDICTED: Homo sapiens hypothetical protein |
| | | | LOC100129343 (LOC100129343), mRNA. |
| 7560593 | OSM | 5008 | Homo sapiens oncostatin M (OSM), mRNA. |
| 7610767 | F5 | 2153 | Homo sapiens coagulation factor V (proaccelerin, labile |
| | | | factor) (F5), mRNA. |
| 2070446 | LOC644276 | 644276 | PREDICTED: Homo sapiens similar to splicing |
| | | | coactivator subunit SRm300 (LOC644276), mRNA. |
| 3170241 | LOC651524 | 651524 | PREDICTED: Homo sapiens similar to integrin alpha X |
| | | | precursor (LOC651524), mRNA. |
| 6100379 | LOC648984 | 648984 | PREDICTED: Homo sapiens similar to Baculoviral IAP |
| | | | repeat-containing protein 1 (Neuronal apoptosis |
| | | | inhibitory protein) (LOC648984), mRNA. |
| 5420678 | LOC100132119 | 100132119 | PREDICTED: Homo sapiens misc_RNA |
| | | | (LOC100132119), miscRNA. |
| 2970019 | HIST1H4H | 8365 | Homo sapiens histone cluster 1, H4h (HIST1H4H), |
| | | | mRNA. |
| 1050050 | C9orf45 | 81571 | PREDICTED: Homo sapiens chromosome 9 open |
| | | | reading frame 45 (C9orf45), misc RNA. |
| 3780047 | GBP6 | 163351 | Homo sapiens guanylate binding protein family, member |
| | | | 6 (GBP6), mRNA. |
| 5910064 | TRIM5 | 85363 | Homo sapiens tripartite motif-containing 5 (TRIM5), |
| | | | transcript variant delta, mRNA. |
| 4230102 | SOCS3 | 9021 | Homo sapiens suppressor of cytokine signaling 3 |
| | | | (SOCS3), mRNA. |
| 5690463 | C11orf80 | 79703 | Homo sapiens chromosome 11 open reading frame 80 |
| | | | (C11orf80), mRNA. |
| 3060524 | LOC153561 | 153561 | Homo sapiens hypothetical protein LOC153561 |
| | | | (LOC153561), mRNA. |
| 5340519 | | | Homo sapiens cDNA FLJ12777 fis, clone |
| | | | NT2RP2001720 |
| 2650605 | C4orf18 | 51313 | Homo sapiens chromosome 4 open reading frame 18 |
| | | | (C4orf18), transcript variant 2, mRNA. |
| 7400102 | ASPHD2 | 57168 | Homo sapiens aspartate beta-hydroxylase domain |
| | | | containing 2 (ASPHD2), mRNA. |
| 7160468 | DHRS9 | 10170 | Homo sapiens dehydrogenase/reductase (SDR family) |
| | | | member 9 (DHRS9), transcript variant 1, mRNA. |
| 2120026 | LOC100134300 | 100134300 | PREDICTED: Homo sapiens misc_RNA |
| | | | (LOC100134300), miscRNA. |
| 2140242 | TNFAIP6 | 7130 | Homo sapiens tumor necrosis factor, alpha-induced |
| | | | protein 6 (TNFAIP6), mRNA. |

TABLE 10
List of the 1129 genes

| ProbeID | Symbol | Entrez_Gene_ID | Definition |
|---|---|---|---|
| 4730168 | SARS2 | 54938 | Homo sapiens seryl-tRNA synthetase 2, mitochondrial |
| | | | (SARS2), nuclear gene encoding mitochondrial protein, |
| | | | mRNA. |
| 2900224 | ENTPD4 | 9583 | Homo sapiens ectonucleoside triphosphate |
| | | | diphosphohydrolase 4 (ENTPD4), mRNA. |
| 5360398 | ERI1 | 90459 | Homo sapiens exoribonuclease 1 (ERI1), mRNA. |
| 4390202 | CXCR3 | 2833 | Homo sapiens chemokine (C—X—C motif) receptor 3 |
| | | | (CXCR3), transcript variant A, mRNA. |
| 7560114 | KLHDC8B | 200942 | Homo sapiens kelch domain containing 8B (KLHDC8B), |
| | | | mRNA. |
| 160612 | MGLL | 11343 | Homo sapiens monoglyceride lipase (MGLL), transcript |
| | | | variant 1, mRNA. |
| 2190349 | KCNJ15 | 3772 | Homo sapiens potassium inwardly-rectifying channel, |
| | | | subfamily J, member 15 (KCNJ15), transcript variant 3, |
| | | | mRNA. |
| 4120243 | PSG9 | 5678 | Homo sapiens pregnancy specific beta-1-glycoprotein 9 |
| | | | (PSG9), mRNA. |
| 1500309 | | | Homo sapiens cDNA: FLJ22140 fis, clone HEP20977 |
| 5860669 | LOC100170939 | 100170939 | Homo sapiens glucuronidase, beta pseudogene |
| | | | (LOC100170939), non-coding RNA. |
| 4060717 | LOC400499 | 400499 | Homo sapiens hypothetical gene supported by AK126539 |
| | | | (LOC400499), mRNA. |
| 3440025 | AGPAT4 | 56895 | Homo sapiens 1-acylglycerol-3-phosphate O- |
| | | | acyltransferase 4 (lysophosphatidic acid acyltransferase, |
| | | | delta) (AGPAT4), mRNA. |
| 5260630 | SOCS3 | 9021 | Homo sapiens suppressor of cytokine signaling 3 |
| | | | (SOCS3), mRNA. |
| 540288 | TMEM88 | 92162 | Homo sapiens transmembrane protein 88 (TMEM88), |
| | | | mRNA. |
| 5270487 | LOC643012 | 643012 | PREDICTED: Homo sapiens hypothetical protein |
| | | | LOC643012 (LOC643012), mRNA. |
| 5700725 | EPSTI1 | 94240 | Homo sapiens epithelial stromal interaction 1 (breast) |
| | | | (EPSTI1), transcript variant 2, mRNA. |
| 6550333 | CXCR6 | 10663 | Homo sapiens chemokine (C—X—C motif) receptor 6 |
| | | | (CXCR6), mRNA. |
| 670594 | C2 | 717 | Homo sapiens complement component 2 (C2), mRNA. |
| 3450114 | FAM69B | 138311 | PREDICTED: Homo sapiens family with sequence |
| | | | similarity 69, member B (FAM69B), mRNA. |
| 60706 | SDCCAG3 | 10807 | Homo sapiens serologically defined colon cancer antigen |
| | | | 3 (SDCCAG3), transcript variant 3, mRNA. |
| 6100356 | ALPL | 249 | Homo sapiens alkaline phosphatase, liver/bone/kidney |
| | | | (ALPL), transcript variant 1, mRNA. |
| 5220538 | WDFY3 | 23001 | Homo sapiens WD repeat and FYVE domain containing |
| | | | 3 (WDFY3), transcript variant 2, mRNA. |
| 1340039 | TFPI | 7035 | Homo sapiens tissue factor pathway inhibitor |
| | | | (lipoprotein-associated coagulation inhibitor) (TFPI), |
| | | | transcript variant 1, mRNA. |
| 4850731 | TFPI | 7035 | Homo sapiens tissue factor pathway inhibitor |
| | | | (lipoprotein-associated coagulation inhibitor) (TFPI), |
| | | | transcript variant 1, mRNA. |
| 2070646 | GPR84 | 53831 | Homo sapiens G protein-coupled receptor 84 (GPR84), |
| | | | mRNA. |
| 3520398 | IL28RA | 163702 | Homo sapiens interleukin 28 receptor, alpha (interferon, |
| | | | lambda receptor) (IL28RA), transcript variant 1, mRNA. |
| 6650176 | TRMT11 | 60487 | Homo sapiens tRNA methyltransferase 11 homolog (S. cerevisiae) |
| | | | (TRMT11), mRNA. |
| 520162 | HPSE | 10855 | Homo sapiens heparanase (HPSE), mRNA. |
| 3850440 | FCER1G | 2207 | Homo sapiens Fc fragment of IgE, high affinity I, |
| | | | receptor for; gamma polypeptide (FCER1G), mRNA. |
| 130181 | ANKRD22 | 118932 | Homo sapiens ankyrin repeat domain 22 (ANKRD22), |
| | | | mRNA. |
| 1010630 | NOP2 | 4839 | Homo sapiens NOP2 nucleolar protein homolog (yeast) |
| | | | (NOP2), transcript variant 1, mRNA. |
| 6960538 | NOP2 | 4839 | Homo sapiens NOP2 nucleolar protein homolog (yeast) |
| | | | (NOP2), transcript variant 1, mRNA. |
| 620136 | LILRA5 | 353514 | Homo sapiens leukocyte immunoglobulin-like receptor, |
| | | | subfamily A (with TM domain), member 5 (LILRA5), |
| | | | transcript variant 3, mRNA. |
| 5860075 | CAMP | 820 | Homo sapiens cathelicidin antimicrobial peptide |
| | | | (CAMP), mRNA. |
| 4120164 | LOC650116 | 650116 | PREDICTED: Homo sapiens hypothetical protein |
| | | | LOC650116 (LOC650116), mRNA. |
| 4290368 | PSTPIP2 | 9050 | Homo sapiens proline-serine-threonine phosphatase |
| | | | interacting protein 2 (PSTPIP2), mRNA. |
| 2360348 | CMPK2 | 129607 | Homo sapiens cytidine monophosphate (UMP-CMP) |
| | | | kinase 2, mitochondrial (CMPK2), nuclear gene encoding |
| | | | mitochondrial protein, mRNA. |
| 2850630 | C19orf48 | 84798 | Homo sapiens chromosome 19 open reading frame 48 |
| | | | (C19orf48), mRNA. |
| 2630195 | VAMP5 | 10791 | Homo sapiens vesicle-associated membrane protein 5 |
| | | | (myobrevin) (VAMP5), mRNA. |
| 3180253 | ELMO2 | 63916 | Homo sapiens engulfment and cell motility 2 (ELMO2), |
| | | | transcript variant 2, mRNA. |
| 3890709 | SMAD7 | 4092 | Homo sapiens SMAD family member 7 (SMAD7), |
| | | | mRNA. |
| 5570398 | FCGR1C | 100132417 | Homo sapiens Fc fragment of IgG, high affinity Ic, |
| | | | receptor (CD64) (FCGR1C), mRNA. |
| 770167 | CD79B | 974 | Homo sapiens CD79b molecule, immunoglobulin- |
| | | | associated beta (CD79B), transcript variant 3, mRNA. |
| 2370228 | CCDC65 | 85478 | Homo sapiens coiled-coil domain containing 65 |
| | | | (CCDC65), mRNA. |
| 510184 | LOC646434 | 646434 | PREDICTED: Homo sapiens hypothetical LOC646434 |
| | | | (LOC646434), mRNA. |
| 3390487 | RECK | 8434 | Homo sapiens reversion-inducing-cysteine-rich protein |
| | | | with kazal motifs (RECK), mRNA. |
| 6620209 | FCGR1B | 2210 | Homo sapiens Fc fragment of IgG, high affinity Ib, |
| | | | receptor (CD64) (FCGR1B), transcript variant 2, mRNA. |
| 5960156 | MOBK1B | 55233 | Homo sapiens MOB1, Mps One Binder kinase activator- |
| | | | like 1B (yeast) (MOBK1B), mRNA. |
| 4480528 | LOC100131139 | 100131139 | PREDICTED: Homo sapiens misc_RNA |
| | | | (LOC100131139), miscRNA. |
| 7560201 | NOC3L | 64318 | Homo sapiens nucleolar complex associated 3 homolog |
| | | | (S. cerevisiae) (NOC3L), mRNA. |
| 130609 | FCGBP | 8857 | Homo sapiens Fc fragment of IgG binding protein |
| | | | (FCGBP), mRNA. |
| 2230575 | LOC642267 | 642267 | PREDICTED: Homo sapiens similar to hypothetical |
| | | | protein LOC284701, transcript variant 2 (LOC642267), |
| | | | mRNA. |
| 20048 | HIST2H2BF | 440689 | Homo sapiens histone cluster 2, H2bf (HIST2H2BF), |
| | | | mRNA. |
| 3990010 | | | AGENCOURT_7914287 NIH_MGC_71 Homo sapiens |
| | | | cDNA clone IMAGE: 6156595 5, mRNA sequence |
| 3800600 | MAGED1 | 9500 | Homo sapiens melanoma antigen family D, 1 |
| | | | (MAGED1), transcript variant 2, mRNA. |
| 1510364 | GBP5 | 115362 | Homo sapiens guanylate binding protein 5 (GBP5), |
| | | | mRNA. |
| 1340241 | C5 | 727 | Homo sapiens complement component 5 (C5), mRNA. |
| 670202 | SESTD1 | 91404 | Homo sapiens SEC14 and spectrin domains 1 (SESTD1), |
| | | | mRNA. |
| 6370768 | ETV7 | 51513 | Homo sapiens ets variant 7 (ETV7), mRNA. |
| 6180497 | LOC400304 | 400304 | PREDICTED: Homo sapiens similar to Golgi |
| | | | autoantigen, golgin subfamily A member 2 (Golgi matrix |
| | | | protein GM130), transcript variant 1 (LOC400304), |
| | | | mRNA. |
| 3440095 | PSG3 | 5671 | Homo sapiens pregnancy specific beta-1-glycoprotein 3 |
| | | | (PSG3), mRNA. |
| 4890382 | ILVBL | 10994 | Homo sapiens ilvB (bacterial acetolactate synthase)-like |
| | | | (ILVBL), mRNA. |
| 50445 | LOC728262 | 728262 | PREDICTED: Homo sapiens hypothetical LOC728262 |
| | | | (LOC728262), mRNA. |
| 6580041 | GNLY | 10578 | Homo sapiens granulysin (GNLY), transcript variant |
| | | | NKG5, mRNA. |
| 1030278 | C10orf33 | 84795 | Homo sapiens chromosome 10 open reading frame 33 |
| | | | (C10orf33), mRNA. |
| 580347 | LOC388572 | 388572 | PREDICTED: Homo sapiens hypothetical LOC388572 |
| | | | (LOC388572), mRNA. |
| 6400148 | SELM | 140606 | Homo sapiens selenoprotein M (SELM), mRNA. |
| 6250064 | IRF1 | 3659 | Homo sapiens interferon regulatory factor 1 (IRF1), |
| | | | mRNA. |
| 2850437 | LOC441124 | 441124 | PREDICTED: Homo sapiens hypothetical LOC441124 |
| | | | (LOC441124), mRNA. |
| 1740382 | GCH1 | 2643 | Homo sapiens GTP cyclohydrolase 1 (GCH1), transcript |
| | | | variant 1, mRNA. |
| 4050039 | FCGR3A | 2214 | Homo sapiens Fc fragment of IgG, low affinity IIIa, |
| | | | receptor (CD16a) (FCGR3A), transcript variant 1, |
| | | | mRNA. |
| 6100474 | EVI2A | 2123 | Homo sapiens ecotropic viral integration site 2A |
| | | | (EVI2A), transcript variant 1, mRNA. |
| 150064 | ABHD14B | 84836 | Homo sapiens abhydrolase domain containing 14B |
| | | | (ABHD14B), mRNA. |
| 4280632 | GAS6 | 2621 | Homo sapiens growth arrest-specific 6 (GAS6), mRNA. |
| 4780463 | RAB6B | 51560 | Homo sapiens RAB6B, member RAS oncogene family |
| | | | (RAB6B), mRNA. |
| 1850220 | DAPP1 | 27071 | Homo sapiens dual adaptor of phosphotyrosine and 3- |
| | | | phosphoinositides (DAPP1), mRNA. |
| 6330278 | MAP2K5 | 5607 | Homo sapiens mitogen-activated protein kinase kinase 5 |
| | | | (MAP2K5), transcript variant B, mRNA. |
| 7560504 | | | ai88d03.s1 Soares_parathyroid_tumor_NbHPA Homo |
| | | | sapiens cDNA clone IMAGE: 1387877 3, mRNA |
| | | | sequence |
| 3800753 | CDCA7L | 55536 | Homo sapiens cell division cycle associated 7-like |
| | | | (CDCA7L), mRNA. |
| 1470091 | IL15 | 3600 | Homo sapiens interleukin 15 (IL15), transcript variant 1, |
| | | | mRNA. |
| 2490315 | | | Homo sapiens cDNA: FLJ23098 fis, clone LNG07440 |
| 1190367 | IER3 | 8870 | Homo sapiens immediate early response 3 (IER3), |
| | | | mRNA. |
| 5270551 | LOC100129267 | 100129267 | PREDICTED: Homo sapiens misc_RNA |
| | | | (LOC100129267), miscRNA. |
| 110639 | MAPK14 | 1432 | Homo sapiens mitogen-activated protein kinase 14 |
| | | | (MAPK14), transcript variant 2, mRNA. |
| 6420296 | MRPL2 | 51069 | Homo sapiens mitochondrial ribosomal protein L2 |
| | | | (MRPL2), nuclear gene encoding mitochondrial protein, |
| | | | mRNA. |
| 3450349 | CSF2RB | 1439 | Homo sapiens colony stimulating factor 2 receptor, beta, |
| | | | low-affinity (granulocyte-macrophage) (CSF2RB), |
| | | | mRNA. |
| 3180220 | GPR109B | 8843 | Homo sapiens G protein-coupled receptor 109B |
| | | | (GPR109B), mRNA. |
| 4860255 | ASPRV1 | 151516 | Homo sapiens aspartic peptidase, retroviral-like 1 |
| | | | (ASPRV1), mRNA. |
| 3390097 | LOC728093 | 728093 | PREDICTED: Homo sapiens misc_RNA (LOC728093), |
| | | | miscRNA. |
| 4610471 | HIST2H4B | 554313 | Homo sapiens histone cluster 2, H4b (HIST2H4B), |
| | | | mRNA. |
| 5890300 | DNAJC24 | 120526 | Homo sapiens DnaJ (Hsp40) homolog, subfamily C, |
| | | | member 24 (DNAJC24), mRNA. |
| 2970201 | ABHD2 | 11057 | Homo sapiens abhydrolase domain containing 2 |
| | | | (ABHD2), transcript variant 2, mRNA. |
| 2070184 | NTHL1 | 4913 | Homo sapiens nth endonuclease III-like 1 (E. coli) |
| | | | (NTHL1), mRNA. |
| 2470142 | ZFP82 | 284406 | Homo sapiens zinc finger protein 82 homolog (mouse) |
| | | | (ZFP82), mRNA. |
| 5340324 | FUT7 | 2529 | Homo sapiens fucosyltransferase 7 (alpha (1,3) |
| | | | fucosyltransferase) (FUT7), mRNA. |
| 6560156 | DUSP3 | 1845 | Homo sapiens dual specificity phosphatase 3 (vaccinia |
| | | | virus phosphatase VH1-related) (DUSP3), mRNA. |
| 5960398 | NT5E | 4907 | Homo sapiens 5′-nucleotidase, ecto (CD73) (NT5E), |
| | | | mRNA. |
| 6370347 | LOC100129541 | 100129541 | PREDICTED: Homo sapiens misc_RNA |
| | | | (LOC100129541), miscRNA. |
| 6040577 | SLAMF8 | 56833 | Homo sapiens SLAM family member 8 (SLAMF8), |
| | | | mRNA. |
| 270050 | ECE1 | 1889 | Homo sapiens endothelin converting enzyme 1 (ECE1), |
| | | | mRNA. |
| 7570324 | ID3 | 3399 | Homo sapiens inhibitor of DNA binding 3, dominant |
| | | | negative helix-loop-helix protein (ID3), mRNA. |
| 4200669 | CR1 | 1378 | Homo sapiens complement component (3b/4b) receptor 1 |
| | | | (Knops blood group) (CR1), transcript variant S, mRNA. |
| 3940356 | CR1 | 1378 | Homo sapiens complement component (3b/4b) receptor 1 |
| | | | (Knops blood group) (CR1), transcript variant S, mRNA. |
| 7560039 | TAPBP | 6892 | Homo sapiens TAP binding protein (tapasin) (TAPBP), |
| | | | transcript variant 3, mRNA. |
| 5080468 | MBOAT2 | 129642 | PREDICTED: Homo sapiens membrane bound O- |
| | | | acyltransferase domain containing 2 (MBOAT2), mRNA. |
| 4730368 | LOC390183 | 390183 | PREDICTED: Homo sapiens misc_RNA (LOC390183), |
| | | | miscRNA. |
| 4850079 | WDR5 | 11091 | Homo sapiens WD repeat domain 5 (WDR5), transcript |
| | | | variant 1, mRNA. |
| 4640411 | KCND1 | 3750 | Homo sapiens potassium voltage-gated channel, Shal- |
| | | | related subfamily, member 1 (KCND1), mRNA. |
| 2030309 | SERPING1 | 710 | Homo sapiens serpin peptidase inhibitor, clade G (C1 |
| | | | inhibitor), member 1 (SERPING1), transcript variant 2, |
| | | | mRNA. |
| 940369 | LOC100129637 | 100129637 | Homo sapiens hypothetical LOC100129637 |
| | | | (LOC100129637), non-coding RNA. XR_039552 |
| | | | XR_039796 |
| 5960731 | SOD2 | 6648 | Homo sapiens superoxide dismutase 2, mitochondrial |
| | | | (SOD2), nuclear gene encoding mitochondrial protein, |
| | | | transcript variant 1, mRNA. |
| 4850451 | SCARNA3 | 677679 | Homo sapiens small Cajal body-specific RNA 3 |
| | | | (SCARNA3), guide RNA. |
| 7570196 | TSPAN9 | 10867 | Homo sapiens tetraspanin 9 (TSPAN9), mRNA. |
| 3170273 | FER1L3 | 26509 | Homo sapiens fer-1-like 3, myoferlin (C. elegans) |
| | | | (FER1L3), transcript variant 2, mRNA. |
| 6940164 | LBH | 81606 | PREDICTED: Homo sapiens hypothetical protein |
| | | | DKFZp566J091 (LBH), mRNA. |
| 4730349 | PNKD | 25953 | Homo sapiens paroxysmal nonkinesiogenic dyskinesia |
| | | | (PNKD), transcript variant 2, mRNA. |
| 7400603 | C15orf54 | 400360 | Homo sapiens chromosome 15 open reading frame 54 |
| | | | (C15orf54), mRNA. |
| 4780044 | LOC389386 | 389386 | PREDICTED: Homo sapiens misc_RNA (LOC389386), |
| | | | partial miscRNA. |
| 580343 | ZNF540 | 163255 | Homo sapiens zinc finger protein 540 (ZNF540), mRNA. |
| 4860259 | OPRL1 | 4987 | Homo sapiens opiate receptor-like 1 (OPRL1), transcript |
| | | | variant 2, mRNA. |
| 5360064 | GNLY | 10578 | Homo sapiens granulysin (GNLY), transcript variant 519, |
| | | | mRNA. |
| 3450414 | GRIPAP1 | 56850 | Homo sapiens GRIP1 associated protein 1 (GRIPAP1), |
| | | | transcript variant 1, mRNA. |
| 6650100 | WDR36 | 134430 | Homo sapiens WD repeat domain 36 (WDR36), mRNA. |
| 3710746 | OXSM | 54995 | Homo sapiens 3-oxoacyl-ACP synthase, mitochondrial |
| | | | (OXSM), nuclear gene encoding mitochondrial protein, |
| | | | mRNA. |
| 4810468 | SELP | 6403 | Homo sapiens selectin P (granule membrane protein |
| | | | 140 kDa, antigen CD62) (SELP), mRNA. |
| 1980382 | MED24 | 9862 | Homo sapiens mediator complex subunit 24 (MED24), |
| | | | transcript variant 1, mRNA. |
| 610598 | CHI3L2 | 1117 | Homo sapiens chitinase 3-like 2 (CHI3L2), transcript |
| | | | variant 1, mRNA. |
| 5690494 | | | AV647906 GLC Homo sapiens cDNA clone GLCBDC04 |
| | | | 3, mRNA sequence |
| 5130025 | LOC100134703 | 100134703 | PREDICTED: Homo sapiens misc_RNA |
| | | | (LOC100134703), partial miscRNA. |
| 2060176 | | | Human mRNA for T-cell receptor alpha-chain J segment, |
| | | | partial cds, clone WADM36A |
| 6370553 | EPHA1 | 2041 | Homo sapiens EPH receptor A1 (EPHA1), mRNA. |
| 4250082 | CHD6 | 84181 | Homo sapiens chromodomain helicase DNA binding |
| | | | protein 6 (CHD6), mRNA. |
| 4120561 | LIMK2 | 3985 | Homo sapiens LIM domain kinase 2 (LIMK2), transcript |
| | | | variant 1, mRNA. |
| 110435 | LOC441550 | 441550 | PREDICTED: Homo sapiens misc_RNA (LOC441550), |
| | | | miscRNA. |
| 50543 | | | Homo sapiens cDNA FLJ41846 fis, clone |
| | | | NT2RI3003162 |
| 7050370 | ANKRD9 | 122416 | Homo sapiens ankyrin repeat domain 9 (ANKRD9), |
| | | | mRNA. |
| 940220 | NOD2 | 64127 | Homo sapiens nucleotide-binding oligomerization |
| | | | domain containing 2 (NOD2), mRNA. |
| 3360491 | MCTP1 | 79772 | Homo sapiens multiple C2 domains, transmembrane 1 |
| | | | (MCTP1), transcript variant L, mRNA. |
| 4010139 | BANK1 | 55024 | Homo sapiens B-cell scaffold protein with ankyrin |
| | | | repeats 1 (BANK1), transcript variant 2, mRNA. |
| 2230678 | ACACB | 32 | Homo sapiens acetyl-Coenzyme A carboxylase beta |
| | | | (ACACB), mRNA. |
| 2570187 | ZNF30 | 90075 | Homo sapiens zinc finger protein 30 (ZNF30), transcript |
| | | | variant 2, mRNA. |
| 2140519 | SOS1 | 6654 | Homo sapiens son of sevenless homolog 1 (Drosophila) |
| | | | (SOS1), mRNA. |
| 5890670 | LOC100131460 | 100131460 | PREDICTED: Homo sapiens misc_RNA |
| | | | (LOC100131460), miscRNA. |
| 3990162 | LOC646909 | 646909 | PREDICTED: Homo sapiens misc_RNA (LOC646909), |
| | | | miscRNA. |
| 150605 | YTHDC2 | 64848 | Homo sapiens YTH domain containing 2 (YTHDC2), |
| | | | mRNA. |
| 4540184 | SYTL4 | 94121 | Homo sapiens synaptotagmin-like 4 (granuphilin-a) |
| | | | (SYTL4), mRNA. |
| 3610719 | SAMD3 | 154075 | Homo sapiens sterile alpha motif domain containing 3 |
| | | | (SAMD3), transcript variant 1, mRNA. |
| 1030167 | SAMD3 | 154075 | Homo sapiens sterile alpha motif domain containing 3 |
| | | | (SAMD3), transcript variant 1, mRNA. |
| 4670592 | MYOF | 26509 | Homo sapiens myoferlin (MYOF), transcript variant 1, |
| | | | mRNA. |
| 2650093 | NFAT5 | 10725 | Homo sapiens nuclear factor of activated T-cells 5, |
| | | | tonicity-responsive (NFAT5), transcript variant 2, |
| | | | mRNA. |
| 2680050 | SIRPG | 55423 | Homo sapiens signal-regulatory protein gamma (SIRPG), |
| | | | transcript variant 2, mRNA. |
| 730008 | | | xv24e03.x1 Soares_NFL_T_GBC_S1 Homo sapiens |
| | | | cDNA clone IMAGE: 2814076 3, mRNA sequence |
| 1450139 | FCGR3B | 2215 | Homo sapiens Fc fragment of IgG, low affinity IIIb, |
| | | | receptor (CD16b) (FCGR3B), mRNA. |
| 7160148 | ZNF329 | 79673 | Homo sapiens zinc finger protein 329 (ZNF329), mRNA. |
| 6550382 | CAMKK2 | 10645 | Homo sapiens calcium/calmodulin-dependent protein |
| | | | kinase kinase 2, beta (CAMKK2), transcript variant 4, |
| | | | mRNA. |
| 5420646 | CETP | 1071 | Homo sapiens cholesteryl ester transfer protein, plasma |
| | | | (CETP), mRNA. |
| 450424 | CXCR7 | 57007 | Homo sapiens chemokine (C—X—C motif) receptor 7 |
| | | | (CXCR7), mRNA. |
| 3830220 | UBASH3A | 53347 | Homo sapiens ubiquitin associated and SH3 domain |
| | | | containing, A (UBASH3A), transcript variant 2, mRNA. |
| 1470639 | LOC100129905 | 100129905 | PREDICTED: Homo sapiens similar to hCG2027326 |
| | | | (LOC100129905), mRNA. |
| 1510619 | LOC100129905 | 100129905 | PREDICTED: Homo sapiens similar to hCG2027326 |
| | | | (LOC100129905), mRNA. |
| 5560246 | TPM1 | 7168 | Homo sapiens tropomyosin 1 (alpha) (TPM1), transcript |
| | | | variant 3, mRNA. |
| 3840463 | TADA1L | 117143 | Homo sapiens transcriptional adaptor 1 (HFI1 homolog, |
| | | | yeast)-like (TADA1L), mRNA. |
| 5360156 | IFITM1 | 8519 | Homo sapiens interferon induced transmembrane protein |
| | | | 1 (9-27) (IFITM1), mRNA. |
| 3830228 | GPR109A | 338442 | Homo sapiens G protein-coupled receptor 109A |
| | | | (GPR109A), mRNA. |
| 5860224 | CD244 | 51744 | Homo sapiens CD244 molecule, natural killer cell |
| | | | receptor 2B4 (CD244), mRNA. |
| 4590424 | PDCD2L | 84306 | Homo sapiens programmed cell death 2-like (PDCD2L), |
| | | | mRNA. |
| 2510220 | IFI35 | 3430 | Homo sapiens interferon-induced protein 35 (IFI35), |
| | | | mRNA. |
| 1470382 | IRF7 | 3665 | Homo sapiens interferon regulatory factor 7 (IRF7), |
| | | | transcript variant b, mRNA. |
| 6400176 | IRF7 | 3665 | Homo sapiens interferon regulatory factor 7 (IRF7), |
| | | | transcript variant b, mRNA. |
| 3400392 | FPR2 | 2358 | Homo sapiens formyl peptide receptor 2 (FPR2), |
| | | | transcript variant 1, mRNA. |
| 5310445 | KREMEN1 | 83999 | Homo sapiens kringle containing transmembrane protein |
| | | | 1 (KREMEN1), transcript variant 4, mRNA. |
| 3390301 | KREMEN1 | 83999 | Homo sapiens kringle containing transmembrane protein |
| | | | 1 (KREMEN1), transcript variant 4, mRNA. |
| 5310754 | VNN1 | 8876 | Homo sapiens vanin 1 (VNN1), mRNA. |
| 6280053 | SMARCAD1 | 56916 | Homo sapiens SWI/SNF-related, matrix-associated actin- |
| | | | dependent regulator of chromatin, subfamily a, containing |
| | | | DEAD/H box 1 (SMARCAD1), mRNA. |
| 6840035 | GBP1 | 2633 | Homo sapiens guanylate binding protein 1, interferon- |
| | | | inducible, 67 kDa (GBP1), mRNA. |
| 2190148 | GBP1 | 2633 | Homo sapiens guanylate binding protein 1, interferon- |
| | | | inducible, 67 kDa (GBP1), mRNA. |
| 4260368 | UBE2C | 11065 | Homo sapiens ubiquitin-conjugating enzyme E2C |
| | | | (UBE2C), transcript variant 3, mRNA. |
| 7320689 | LOC100133422 | 100133422 | PREDICTED: Homo sapiens hypothetical protein |
| | | | LOC100133422 (LOC100133422), mRNA. |
| 6550164 | DEFA4 | 1669 | Homo sapiens defensin, alpha 4, corticostatin (DEFA4), |
| | | | mRNA. |
| 240053 | GCH1 | 2643 | Homo sapiens GTP cyclohydrolase 1 (GCH1), transcript |
| | | | variant 4, mRNA. |
| 2510082 | GCET2 | 257144 | Homo sapiens germinal center expressed transcript 2 |
| | | | (GCET2), transcript variant 1, mRNA. |
| 5220706 | FLJ33630 | 644873 | Homo sapiens hypothetical LOC644873 (FLJ33630), |
| | | | non-coding RNA. |
| 3800398 | FBXO6 | 26270 | Homo sapiens F-box protein 6 (FBXO6), mRNA. |
| 5900524 | LACTB | 114294 | Homo sapiens lactamase, beta (LACTB), nuclear gene |
| | | | encoding mitochondrial protein, transcript variant 2, |
| | | | mRNA. |
| 1570367 | IL18R1 | 8809 | Homo sapiens interleukin 18 receptor 1 (IL18R1), |
| | | | mRNA. |
| 840037 | | | UI-H-BI2-ahe-g-01-0-UI.s1 NCI_CGAP_Sub4 Homo |
| | | | sapiens cDNA clone IMAGE: 2726592 3, mRNA |
| | | | sequence |
| 5550026 | RBM15B | 29890 | Homo sapiens RNA binding motif protein 15B |
| | | | (RBM15B), mRNA. |
| 780324 | ZNF337 | 26152 | Homo sapiens zinc finger protein 337 (ZNF337), mRNA. |
| 650753 | AFF3 | 3899 | Homo sapiens AF4/FMR2 family, member 3 (AFF3), |
| | | | transcript variant 2, mRNA. |
| 6370273 | CTPS | 1503 | Homo sapiens CTP synthase (CTPS), mRNA. |
| 1430711 | KLF12 | 11278 | Homo sapiens Kruppel-like factor 12 (KLF12), mRNA. |
| 4860280 | | | Homo sapiens cDNA FLJ31407 fis, clone |
| | | | NT2NE2000137 |
| 520553 | NCALD | 83988 | Homo sapiens neurocalcin delta (NCALD), transcript |
| | | | variant 3, mRNA. |
| 1400373 | SLA | 6503 | Homo sapiens Src-like-adaptor (SLA), transcript variant |
| | | | 1, mRNA. |
| 3310682 | DOCK5 | 80005 | Homo sapiens dedicator of cytokinesis 5 (DOCK5), |
| | | | mRNA. |
| 6980025 | LOC730546 | 730546 | PREDICTED: Homo sapiens hypothetical protein |
| | | | LOC730546 (LOC730546), mRNA. |
| 1570129 | TRAFD1 | 10906 | Homo sapiens TRAF-type zinc finger domain containing |
| | | | 1 (TRAFD1), mRNA. |
| 2480685 | LOC100131726 | 100131726 | Homo sapiens HCC-related HCC-C11_v3 |
| | | | (LOC100131726), non-coding RNA. |
| 380437 | MGC52282 | 124221 | Homo sapiens hypothetical protein MGC52282 |
| | | | (MGC52282), mRNA. |
| 1260228 | CLEC5A | 23601 | Homo sapiens C-type lectin domain family 5, member A |
| | | | (CLEC5A), mRNA. |
| 3870458 | BRDG1 | 26228 | Homo sapiens BCR downstream signaling 1 (BRDG1), |
| | | | mRNA. |
| 3440377 | PCGF5 | 84333 | Homo sapiens polycomb group ring finger 5 (PCGF5), |
| | | | mRNA. |
| 7560136 | WDR92 | 116143 | Homo sapiens WD repeat domain 92 (WDR92), mRNA. |
| 6200577 | CREB5 | 9586 | Homo sapiens cAMP responsive element binding protein |
| | | | 5 (CREB5), transcript variant 1, mRNA. |
| 2480075 | ATF7IP2 | 80063 | Homo sapiens activating transcription factor 7 interacting |
| | | | protein 2 (ATF7IP2), mRNA. |
| 5090632 | LGALS9 | 3965 | Homo sapiens lectin, galactoside-binding, soluble, 9 |
| | | | (LGALS9), transcript variant 1, mRNA. |
| 4730059 | BATF2 | 116071 | Homo sapiens basic leucine zipper transcription factor, |
| | | | ATF-like 2 (BATF2), mRNA. |
| 510100 | CBY1 | 25776 | Homo sapiens chibby homolog 1 (Drosophila) (CBY1), |
| | | | transcript variant 1, mRNA. |
| 6020709 | LOC100129674 | 100129674 | PREDICTED: Homo sapiens similar to hCG2002091 |
| | | | (LOC100129674), mRNA. |
| 3420241 | SLC2A14 | 144195 | Homo sapiens solute carrier family 2 (facilitated glucose |
| | | | transporter), member 14 (SLC2A14), mRNA. |
| 7050519 | CLEC4D | 338339 | Homo sapiens C-type lectin domain family 4, member D |
| | | | (CLEC4D), mRNA. |
| 7510647 | CACNA1E | 777 | Homo sapiens calcium channel, voltage-dependent, R |
| | | | type, alpha 1E subunit (CACNA1E), mRNA. |
| 2570328 | TRIB2 | 28951 | Homo sapiens tribbles homolog 2 (Drosophila) (TRIB2), |
| | | | mRNA. |
| 70180 | LOC100133823 | 100133823 | PREDICTED: Homo sapiens misc_RNA |
| | | | (LOC100133823), miscRNA. |
| 4640768 | VPREB3 | 29802 | Homo sapiens pre-B lymphocyte gene 3 (VPREB3), |
| | | | mRNA. |
| 4480279 | Sep-01 | 1731 | Homo sapiens septin 1 (SEPT1), mRNA. XM_944608 |
| | | | XM_944610 |
| 2450392 | LOC642780 | 642780 | PREDICTED: Homo sapiens similar to hypothetical |
| | | | protein LOC284701, transcript variant 2 (LOC642780), |
| | | | mRNA. |
| 6590220 | MEGF6 | 1953 | Homo sapiens multiple EGF-like-domains 6 (MEGF6), |
| | | | mRNA. |
| 2630224 | LOC93622 | 93622 | PREDICTED: Homo sapiens hypothetical protein |
| | | | BC006130 (LOC93622), misc RNA. |
| 3840092 | PFAS | 5198 | Homo sapiens phosphoribosylformylglycinamidine |
| | | | synthase (FGAR amidotransferase) (PFAS), mRNA. |
| 2340327 | CD320 | 51293 | Homo sapiens CD320 molecule (CD320), mRNA. |
| 7320241 | FLJ37307 | 283521 | PREDICTED: Homo sapiens hypothetical protein |
| | | | FLJ37307 (FLJ37307), misc RNA. |
| 60500 | RIOK1 | 83732 | Homo sapiens RIO kinase 1 (yeast) (RIOK1), transcript |
| | | | variant 1, mRNA. |
| 620019 | RPS26P10 | 401470 | PREDICTED: Homo sapiens ribosomal protein S26 |
| | | | pseudogene 10 (RPS26P10), mRNA. |
| 1070189 | AXIN2 | 8313 | Homo sapiens axin 2 (conductin, axil) (AXIN2), mRNA. |
| 6510735 | DDX60L | 91351 | Homo sapiens DEAD (Asp-Glu-Ala-Asp) box |
| | | | polypeptide 60-like (DDX60L), mRNA. |
| 6270500 | CARD16 | 114769 | Homo sapiens caspase recruitment domain family, |
| | | | member 16 (CARD16), transcript variant 1, mRNA. |
| 730528 | PLAUR | 5329 | Homo sapiens plasminogen activator, urokinase receptor |
| | | | (PLAUR), transcript variant 1, mRNA. |
| 5050382 | SPARC | 6678 | Homo sapiens secreted protein, acidic, cysteine-rich |
| | | | (osteonectin) (SPARC), mRNA. |
| 4150014 | CEACAM1 | 634 | Homo sapiens carcinoembryonic antigen-related cell |
| | | | adhesion molecule 1 (biliary glycoprotein) (CEACAM1), |
| | | | transcript variant 2, mRNA. |
| 5700753 | CEACAM1 | 634 | Homo sapiens carcinoembryonic antigen-related cell |
| | | | adhesion molecule 1 (biliary glycoprotein) (CEACAM1), |
| | | | transcript variant 2, mRNA. |
| 1980768 | SIAH2 | 6478 | Homo sapiens seven in absentia homolog 2 (Drosophila) |
| | | | (SIAH2), mRNA. |
| 5360041 | DDAH2 | 23564 | Homo sapiens dimethylarginine dimethylaminohydrolase |
| | | | 2 (DDAH2), mRNA. |
| 5960343 | PRIC285 | 85441 | Homo sapiens peroxisomal proliferator-activated receptor |
| | | | A interacting complex 285 (PRIC285), transcript variant |
| | | | 2, mRNA. |
| 3850373 | RHCE | 6006 | Homo sapiens Rh blood group, CcEe antigens (RHCE), |
| | | | transcript variant 4, mRNA. |
| 6560152 | PPP2R3A | 5523 | Homo sapiens protein phosphatase 2 (formerly 2A), |
| | | | regulatory subunit B″, alpha (PPP2R3A), transcript |
| | | | variant 1, mRNA. |
| 1820725 | LRRK2 | 120892 | Homo sapiens leucine-rich repeat kinase 2 (LRRK2), |
| | | | mRNA. |
| 3520291 | LRRK2 | 120892 | Homo sapiens leucine-rich repeat kinase 2 (LRRK2), |
| | | | mRNA. |
| 4390398 | LCN2 | 3934 | Homo sapiens lipocalin 2 (LCN2), mRNA. |
| 1240097 | BAZ1A | 11177 | Homo sapiens bromodomain adjacent to zinc finger |
| | | | domain, 1A (BAZ1A), transcript variant 1, mRNA. |
| 2650113 | NR1I2 | 8856 | Homo sapiens nuclear receptor subfamily 1, group I, |
| | | | member 2 (NR1I2), transcript variant 3, mRNA. |
| 3610647 | URG4 | 55665 | Homo sapiens up-regulated gene 4 (URG4), nuclear gene |
| | | | encoding mitochondrial protein, transcript variant 2, |
| | | | mRNA. |
| 460682 | CCDC132 | 55610 | Homo sapiens coiled-coil domain containing 132 |
| | | | (CCDC132), transcript variant 1, mRNA. |
| 10440 | MARS2 | 92935 | Homo sapiens methionyl-tRNA synthetase 2, |
| | | | mitochondrial (MARS2), nuclear gene encoding |
| | | | mitochondrial protein, mRNA. |
| 2750575 | | | full-length cDNA clone CS0CAP005YH21 of Thymus of |
| | | | Homo sapiens (human) |
| 6350440 | LOC100131572 | 100131572 | PREDICTED: Homo sapiens similar to hCG1783679 |
| | | | (LOC100131572), mRNA. |
| 3060408 | LOC645231 | 645231 | PREDICTED: Homo sapiens misc_RNA (LOC645231), |
| | | | miscRNA. |
| 6200193 | TncRNA | 283131 | PREDICTED: Homo sapiens trophoblast-derived |
| | | | noncoding RNA (TncRNA), misc RNA. |
| 3460053 | SIPA1L2 | 57568 | Homo sapiens signal-induced proliferation-associated 1 |
| | | | like 2 (SIPA1L2), mRNA. |
| 1240270 | LMTK2 | 22853 | Homo sapiens lemur tyrosine kinase 2 (LMTK2), mRNA. |
| 2230619 | HIST1H4E | 8367 | Homo sapiens histone cluster 1, H4e (HIST1H4E), |
| | | | mRNA. |
| 6100347 | PTPRE | 5791 | Homo sapiens protein tyrosine phosphatase, receptor |
| | | | type, E (PTPRE), transcript variant 2, mRNA. |
| 7650497 | ELANE | 1991 | Homo sapiens elastase, neutrophil expressed (ELANE), |
| | | | mRNA. |
| 6840246 | CTSA | 5476 | Homo sapiens cathepsin A (CTSA), transcript variant 1, |
| | | | mRNA. |
| 1580435 | TGM2 | 7052 | Homo sapiens transglutaminase 2 (C polypeptide, |
| | | | protein-glutamine-gamma-glutamyltransferase) (TGM2), |
| | | | transcript variant 1, mRNA. |
| 2970575 | CCL4L2 | 388372 | Homo sapiens chemokine (C-C motif) ligand 4-like 2 |
| | | | (CCL4L2), mRNA. |
| 7400487 | | | QV1-BT0630-040400-132-d11 BT0630 Homo sapiens |
| | | | cDNA, mRNA sequence |
| 3610300 | CCDC58 | 131076 | Homo sapiens coiled-coil domain containing 58 |
| | | | (CCDC58), mRNA. |
| 1510026 | FLVCR2 | 55640 | Homo sapiens feline leukemia virus subgroup C cellular |
| | | | receptor family, member 2 (FLVCR2), mRNA. |
| 7400017 | EPHB6 | 2051 | Homo sapiens EPH receptor B6 (EPHB6), mRNA. |
| 3420593 | LMNB1 | 4001 | Homo sapiens lamin B1 (LMNB1), mRNA. |
| 6660368 | MAP4K1 | 11184 | Homo sapiens mitogen-activated protein kinase kinase |
| | | | kinase kinase 1 (MAP4K1), transcript variant 1, mRNA. |
| 6450692 | PSMB9 | 5698 | Homo sapiens proteasome (prosome, macropain) subunit, |
| | | | beta type, 9 (large multifunctional peptidase 2) (PSMB9), |
| | | | transcript variant 1, mRNA. |
| 4780128 | ATF3 | 467 | Homo sapiens activating transcription factor 3 (ATF3), |
| | | | transcript variant 4, mRNA. |
| 5050347 | CD5 | 921 | Homo sapiens CD5 molecule (CD5), mRNA. |
| 5810278 | SYTL2 | 54843 | Homo sapiens synaptotagmin-like 2 (SYTL2), transcript |
| | | | variant d, mRNA. |
| 5310411 | H2AFJ | 55766 | Homo sapiens H2A histone family, member J (H2AFJ), |
| | | | transcript variant 1, mRNA. |
| 6590131 | HP | 3240 | Homo sapiens haptoglobin (HP), mRNA. |
| 430328 | LOC100130520 | 100130520 | PREDICTED: Homo sapiens similar to immune receptor |
| | | | expressed on myeloid cells 2 (LOC100130520), mRNA. |
| 3420228 | PPP1R13B | 23368 | Homo sapiens protein phosphatase 1, regulatory |
| | | | (inhibitor) subunit 13B (PPP1R13B), mRNA. |
| 6110630 | HIST1H2BK | 85236 | Homo sapiens histone cluster 1, H2bk (HIST1H2BK), |
| | | | mRNA. |
| 5050402 | HIST1H2BK | 85236 | Homo sapiens histone cluster 1, H2bk (HIST1H2BK), |
| | | | mRNA. |
| 4670414 | TMEM140 | 55281 | Homo sapiens transmembrane protein 140 (TMEM140), |
| | | | mRNA. |
| 3870301 | SORBS3 | 10174 | Homo sapiens sorbin and SH3 domain containing 3 |
| | | | (SORBS3), transcript variant 1, mRNA. |
| 4390487 | RAB13 | 5872 | Homo sapiens RAB13, member RAS oncogene family |
| | | | (RAB13), mRNA. |
| 6330471 | BLK | 640 | Homo sapiens B lymphoid tyrosine kinase (BLK), |
| | | | mRNA. |
| 2190019 | CD160 | 11126 | Homo sapiens CD160 molecule (CD160), mRNA. |
| 3360026 | PLXNA4 | 91584 | Homo sapiens plexin A4 (PLXNA4), transcript variant 2, |
| | | | mRNA. |
| 1340403 | LOC100128460 | 100128460 | PREDICTED: Homo sapiens misc_RNA |
| | | | (LOC100128460), miscRNA. |
| 7210136 | CD19 | 930 | Homo sapiens CD19 molecule (CD19), mRNA. |
| 3440360 | ZNF438 | 220929 | Homo sapiens zinc finger protein 438 (ZNF438), mRNA. |
| 6220075 | GPR177 | 79971 | Homo sapiens G protein-coupled receptor 177 (GPR177), |
| | | | transcript variant 1, mRNA. |
| 2100458 | LOC729010 | 729010 | PREDICTED: Homo sapiens hypothetical LOC729010 |
| | | | (LOC729010), mRNA. |
| 7550280 | C3orf17 | 25871 | Homo sapiens chromosome 3 open reading frame 17 |
| | | | (C3orf17), transcript variant 2, mRNA. |
| 2810470 | C6orf25 | 80739 | Homo sapiens chromosome 6 open reading frame 25 |
| | | | (C6orf25), transcript variant 4, mRNA. |
| 4850168 | SLC22A4 | 6583 | Homo sapiens solute carrier family 22 (organic |
| | | | cation/ergothioneine transporter), member 4 (SLC22A4), |
| | | | mRNA. |
| 4730209 | PHC1 | 1911 | Homo sapiens polyhomeotic homolog 1 (Drosophila) |
| | | | (PHC1), mRNA. |
| 6480059 | ACTA2 | 59 | Homo sapiens actin, alpha 2, smooth muscle, aorta |
| | | | (ACTA2), mRNA. |
| 3780709 | KIAA1632 | 57724 | Homo sapiens KIAA1632 (KIAA1632), mRNA. |
| 6100136 | CASP4 | 837 | Homo sapiens caspase 4, apoptosis-related cysteine |
| | | | peptidase (CASP4), transcript variant gamma, mRNA. |
| 6040398 | SLC22A4 | 6583 | Homo sapiens solute carrier family 22 (organic |
| | | | cation/ergothioneine transporter), member 4 (SLC22A4), |
| | | | mRNA. |
| 5910543 | GLOD4 | 51031 | Homo sapiens glyoxalase domain containing 4 (GLOD4), |
| | | | mRNA. |
| 2710452 | POU2AF1 | 5450 | Homo sapiens POU class 2 associating factor 1 |
| | | | (POU2AF1), mRNA. |
| 6420750 | TLR2 | 7097 | Homo sapiens toll-like receptor 2 (TLR2), mRNA. |
| 6590669 | RAB24 | 53917 | Homo sapiens RAB24, member RAS oncogene family |
| | | | (RAB24), transcript variant 1, mRNA. |
| 5910300 | RAB24 | 53917 | Homo sapiens RAB24, member RAS oncogene family |
| | | | (RAB24), transcript variant 1, mRNA. |
| 5270753 | ARG1 | 383 | Homo sapiens arginase, liver (ARG1), mRNA. |
| 2450364 | VNN3 | 55350 | Homo sapiens vanin 3 (VNN3), transcript variant 2, |
| | | | mRNA. |
| 5360025 | ST8SIA1 | 6489 | Homo sapiens ST8 alpha-N-acetyl-neuraminide alpha- |
| | | | 2,8-sialyltransferase 1 (ST8SIA1), mRNA. |
| 2760689 | MAGEE1 | 57692 | Homo sapiens melanoma antigen family E, 1 (MAGEE1), |
| | | | mRNA. |
| 6650743 | TSPYL2 | 64061 | Homo sapiens TSPY-like 2 (TSPYL2), mRNA. |
| 1580358 | WDFY3 | 23001 | Homo sapiens WD repeat and FYVE domain containing |
| | | | 3 (WDFY3), transcript variant 1, mRNA. |
| 3400458 | MSRB3 | 253827 | Homo sapiens methionine sulfoxide reductase B3 |
| | | | (MSRB3), transcript variant 1, mRNA. |
| 7100519 | CEP57 | 9702 | Homo sapiens centrosomal protein 57 kDa (CEP57), |
| | | | mRNA. |
| 2350189 | GBP3 | 2635 | Homo sapiens guanylate binding protein 3 (GBP3), |
| | | | mRNA. |
| 540091 | STEAP4 | 79689 | Homo sapiens STEAP family member 4 (STEAP4), |
| | | | mRNA. |
| 5910615 | TCTN3 | 26123 | Homo sapiens tectonic family member 3 (TCTN3), |
| | | | mRNA. |
| 1570075 | FBXO4 | 26272 | Homo sapiens F-box protein 4 (FBXO4), transcript |
| | | | variant 1, mRNA. |
| 5910719 | GATA3 | 2625 | Homo sapiens GATA binding protein 3 (GATA3), |
| | | | transcript variant 2, mRNA. |
| 3420373 | SOD2 | 6648 | Homo sapiens superoxide dismutase 2, mitochondrial |
| | | | (SOD2), nuclear gene encoding mitochondrial protein, |
| | | | transcript variant 3, mRNA. |
| 6280458 | BCL6 | 604 | Homo sapiens B-cell CLL/lymphoma 6 (zinc finger |
| | | | protein 51) (BCL6), transcript variant 1, mRNA. |
| 5050086 | MPL | 4352 | Homo sapiens myeloproliferative leukemia virus |
| | | | oncogene (MPL), mRNA. |
| 3460376 | RTN2 | 6253 | Homo sapiens reticulon 2 (RTN2), transcript variant 3, |
| | | | mRNA. |
| 2060040 | ADORA2B | 136 | Homo sapiens adenosine A2b receptor (ADORA2B), |
| | | | mRNA. |
| 1660681 | CCDC102A | 92922 | Homo sapiens coiled-coil domain containing 102A |
| | | | (CCDC102A), mRNA. |
| 1410278 | PF4V1 | 5197 | Homo sapiens platelet factor 4 variant 1 (PF4V1), |
| | | | mRNA. |
| 4250630 | KLRF1 | 51348 | Homo sapiens killer cell lectin-like receptor subfamily F, |
| | | | member 1 (KLRF1), mRNA. |
| 6760681 | LOC650761 | 650761 | PREDICTED: Homo sapiens similar to T-cell receptor |
| | | | alpha chain V region CTL-L17 precursor (LOC650761), |
| | | | mRNA. |
| 6650333 | LOC653867 | 653867 | PREDICTED: Homo sapiens similar to Occludin |
| | | | (LOC653867), mRNA. |
| 2690477 | LOC648710 | 648710 | PREDICTED: Homo sapiens similar to leukocyte |
| | | | immunoglobulin-like receptor, subfamily B (with TM and |
| | | | ITIM domains), member 3 (LOC648710), mRNA. |
| 5810685 | THBS1 | 7057 | Homo sapiens thrombospondin 1 (THBS1), mRNA. |
| 630255 | LOC652578 | 652578 | PREDICTED: Homo sapiens similar to Fc fragment of |
| | | | IgG, low affinity IIIa, receptor for (CD16) (LOC652578), |
| | | | mRNA. |
| 6900014 | PHAX | 51808 | Homo sapiens phosphorylated adaptor for RNA export |
| | | | (PHAX), mRNA. |
| 5890075 | LIN7A | 8825 | Homo sapiens lin-7 homolog A (C. elegans) (LIN7A), |
| | | | mRNA. |
| 3360431 | ZAK | 51776 | Homo sapiens sterile alpha motif and leucine zipper |
| | | | containing kinase AZK (ZAK), transcript variant 1, |
| | | | mRNA. |
| 7570184 | LOC283663 | 283663 | Homo sapiens hypothetical LOC283663 (LOC283663), |
| | | | non-coding RNA. |
| 5390328 | KCNJ15 | 3772 | Homo sapiens potassium inwardly-rectifying channel, |
| | | | subfamily J, member 15 (KCNJ15), transcript variant 1, |
| | | | mRNA. |
| 1050215 | KCNJ15 | 3772 | Homo sapiens potassium inwardly-rectifying channel, |
| | | | subfamily J, member 15 (KCNJ15), transcript variant 1, |
| | | | mRNA. |
| 5290523 | ARHGAP25 | 9938 | Homo sapiens Rho GTPase activating protein 25 |
| | | | (ARHGAP25), transcript variant 1, mRNA. |
| 4570164 | LOC389386 | 389386 | PREDICTED: Homo sapiens misc_RNA (LOC389386), |
| | | | partial miscRNA. |
| 4390347 | NBR1 | 4077 | Homo sapiens neighbor of BRCA1 gene 1 (NBR1), |
| | | | transcript variant 1, mRNA. |
| 4260309 | MAST4 | 375449 | Homo sapiens microtubule associated serine/threonine |
| | | | kinase family member 4 (MAST4), transcript variant 2, |
| | | | mRNA. |
| 4920338 | | | BX106903 Soares_NFL_T_GBC_S1 Homo sapiens |
| | | | cDNA clone IMAGp998J154516, mRNA sequence |
| 4640367 | SFI1 | 9814 | Homo sapiens Sfi1 homolog, spindle assembly associated |
| | | | (yeast) (SFI1), transcript variant 1, mRNA. |
| 6020097 | SERPINA1 | 5265 | Homo sapiens serpin peptidase inhibitor, clade A (alpha-1 |
| | | | antiproteinase, antitrypsin), member 1 (SERPINA1), |
| | | | transcript variant 2, mRNA. |
| 3850202 | IGSF6 | 10261 | Homo sapiens immunoglobulin superfamily, member 6 |
| | | | (IGSF6), mRNA. |
| 620403 | LOC400759 | 400759 | Homo sapiens similar to Interferon-induced guanylate- |
| | | | binding protein 1 (GTP-binding protein 1) (Guanine |
| | | | nucleotide-binding protein 1) (HuGBP-1) (LOC400759) |
| | | | on chromosome 1. |
| 6280470 | LOC728519 | 728519 | PREDICTED: Homo sapiens similar to Baculoviral IAP |
| | | | repeat-containing protein 1 (Neuronal apoptosis |
| | | | inhibitory protein) (LOC728519), mRNA. |
| 7610671 | TOP1MT | 116447 | PREDICTED: Homo sapiens topoisomerase (DNA) I, |
| | | | mitochondrial (TOP1MT), mRNA. |
| 6020706 | PLGLB1 | 5343 | Homo sapiens plasminogen-like B1 (PLGLB1), mRNA. |
| 2630577 | CAV2 | 858 | Homo sapiens caveolin 2 (CAV2), transcript variant 1, |
| | | | mRNA. |
| 5910553 | CAV2 | 858 | Homo sapiens caveolin 2 (CAV2), transcript variant 1, |
| | | | mRNA. |
| 6250010 | GPRIN3 | 285513 | Homo sapiens GPRIN family member 3 (GPRIN3), |
| | | | mRNA. |
| 160070 | C16orf30 | 79652 | Homo sapiens chromosome 16 open reading frame 30 |
| | | | (C16orf30), mRNA. |
| 5570114 | GADD45G | 10912 | Homo sapiens growth arrest and DNA-damage-inducible, |
| | | | gamma (GADD45G), mRNA. |
| 1300139 | GNG7 | 2788 | Homo sapiens guanine nucleotide binding protein (G |
| | | | protein), gamma 7 (GNG7), mRNA. |
| 4890181 | RAP1GAP | 5909 | Homo sapiens RAP1 GTPase activating protein |
| | | | (RAP1GAP), mRNA. |
| 2710091 | FCGR2C | 9103 | Homo sapiens Fc fragment of IgG, low affinity IIc, |
| | | | receptor for (CD32) (FCGR2C), transcript variant 3, |
| | | | mRNA. |
| 5570142 | CEP78 | 84131 | Homo sapiens centrosomal protein 78 kDa (CEP78), |
| | | | transcript variant 1, mRNA. |
| 5080201 | LOC642946 | 642946 | PREDICTED: Homo sapiens hypothetical protein |
| | | | LOC642945, transcript variant 2 (LOC642946), mRNA. |
| 6840020 | TNFSF13B | 10673 | Homo sapiens tumor necrosis factor (ligand) superfamily, |
| | | | member 13b (TNFSF13B), transcript variant 1, mRNA. |
| 620075 | UQCC | 55245 | Homo sapiens ubiquinol-cytochrome c reductase complex |
| | | | chaperone (UQCC), nuclear gene encoding mitochondrial |
| | | | protein, transcript variant 2, mRNA. |
| 2030746 | TAL1 | 6886 | Homo sapiens T-cell acute lymphocytic leukemia 1 |
| | | | (TAL1), mRNA. |
| 5490743 | KIF1B | 23095 | Homo sapiens kinesin family member 1B (KIF1B), |
| | | | transcript variant 2, mRNA. |
| 6110634 | ERLIN1 | 10613 | Homo sapiens ER lipid raft associated 1 (ERLIN1), |
| | | | mRNA. |
| 5810253 | POLA1 | 5422 | Homo sapiens polymerase (DNA directed), alpha 1, |
| | | | catalytic subunit (POLA1), mRNA. |
| 430050 | BAMBI | 25805 | Homo sapiens BMP and activin membrane-bound |
| | | | inhibitor homolog (Xenopus laevis) (BAMBI), mRNA. |
| 2230201 | TARBP1 | 6894 | Homo sapiens TAR (HIV-1) RNA binding protein 1 |
| | | | (TARBPI), mRNA. |
| 2360719 | IRAK3 | 11213 | Homo sapiens interleukin-1 receptor-associated kinase 3 |
| | | | (IRAK3), mRNA. |
| 4880537 | LIMA1 | 51474 | Homo sapiens LIM domain and actin binding 1 (LIMA1), |
| | | | mRNA. |
| 1050020 | NLRC4 | 58484 | Homo sapiens NLR family, CARD domain containing 4 |
| | | | (NLRC4), mRNA. |
| 450753 | HIST1H2BC | 8347 | Homo sapiens histone cluster 1, H2bc (HIST1H2BC), |
| | | | mRNA. |
| 5870192 | SKAP1 | 8631 | Homo sapiens src kinase associated phosphoprotein 1 |
| | | | (SKAP1), transcript variant 2, mRNA. |
| 780564 | LOC100132413 | 100132413 | PREDICTED: Homo sapiens similar to hCG1782458 |
| | | | (LOC100132413), mRNA. |
| 2070193 | LOC100128805 | 100128805 | PREDICTED: Homo sapiens hypothetical protein |
| | | | LOC100128805 (LOC100128805), mRNA. |
| 3390121 | CASP5 | 838 | Homo sapiens caspase 5, apoptosis-related cysteine |
| | | | peptidase (CASP5), mRNA. |
| 5890386 | LOC642103 | 642103 | PREDICTED: Homo sapiens similar to Maltase- |
| | | | glucoamylase, intestinal (LOC642103), mRNA. |
| 2900524 | CCR2 | 1231 | Homo sapiens chemokine (C-C motif) receptor 2 (CCR2), |
| | | | transcript variant B, mRNA. |
| 1010544 | GAR1 | 54433 | Homo sapiens GAR1 ribonucleoprotein homolog (yeast) |
| | | | (GAR1), transcript variant 1, mRNA. |
| 2060047 | B4GALT5 | 9334 | Homo sapiens UDP-Gal:betaGlcNAc beta 1,4- |
| | | | galactosyltransferase, polypeptide 5 (B4GALT5), mRNA. |
| 4760433 | C16orf7 | 9605 | Homo sapiens chromosome 16 open reading frame 7 |
| | | | (C16orf7), mRNA. |
| 2370255 | ALDH18A1 | 5832 | Homo sapiens aldehyde dehydrogenase 18 family, |
| | | | member A1 (ALDH18A1), nuclear gene encoding |
| | | | mitochondrial protein, transcript variant 1, mRNA. |
| 20441 | | | Homo sapiens cDNA FLJ20182 fis, clone COLF0190 |
| 4290133 | PACSIN1 | 29993 | Homo sapiens protein kinase C and casein kinase |
| | | | substrate in neurons 1 (PACSIN1), mRNA. |
| 4890722 | CD28 | 940 | Homo sapiens CD28 molecule (CD28), mRNA. |
| 3180392 | MARCO | 8685 | Homo sapiens macrophage receptor with collagenous |
| | | | structure (MARCO), mRNA. |
| 130433 | CTTN | 2017 | Homo sapiens cortactin (CTTN), transcript variant 1, |
| | | | mRNA. |
| 2320689 | LOC653610 | 653610 | PREDICTED: Homo sapiens similar to Histone H2A.o |
| | | | (H2A/o) (H2A.2) (H2a-615) (LOC653610), mRNA. |
| 1230047 | CBS | 875 | Homo sapiens cystathionine-beta-synthase (CBS), |
| | | | mRNA. |
| 580132 | LAPTM4B | 55353 | Homo sapiens lysosomal protein transmembrane 4 beta |
| | | | (LAPTM4B), mRNA. |
| 5130767 | MAPK14 | 1432 | Homo sapiens mitogen-activated protein kinase 14 |
| | | | (MAPK14), transcript variant 1, mRNA. |
| 5700615 | LOC100129552 | 100129552 | PREDICTED: Homo sapiens misc_RNA |
| | | | (LOC100129552), miscRNA. |
| 2480544 | KCNMB1 | 3779 | Homo sapiens potassium large conductance calcium- |
| | | | activated channel, subfamily M, beta member 1 |
| | | | (KCNMB1), mRNA. |
| 3370086 | P2RX7 | 5027 | Homo sapiens purinergic receptor P2X, ligand-gated ion |
| | | | channel, 7 (P2RX7), mRNA. |
| 730672 | HGD | 3081 | Homo sapiens homogentisate 1,2-dioxygenase |
| | | | (homogentisate oxidase) (HGD), mRNA. |
| 840685 | IL1B | 3553 | Homo sapiens interleukin 1, beta (IL1B), mRNA. |
| 7650504 | CTAGE5 | 4253 | Homo sapiens CTAGE family, member 5 (CTAGE5), |
| | | | transcript variant 4, mRNA. |
| 6450397 | CCNB1IP1 | 57820 | Homo sapiens cyclin B1 interacting protein 1 |
| | | | (CCNB1IP1), transcript variant 2, mRNA. |
| 1580161 | NETO2 | 81831 | Homo sapiens neuropilin (NRP) and tolloid (TLL)-like 2 |
| | | | (NETO2), mRNA. |
| 4490403 | IQCB1 | 9657 | Homo sapiens IQ motif containing B1 (IQCB1), |
| | | | transcript variant 3, mRNA. |
| 2000128 | C4BPA | 722 | Homo sapiens complement component 4 binding protein, |
| | | | alpha (C4BPA), mRNA. |
| 4230678 | HIST2H2BE | 8349 | Homo sapiens histone cluster 2, H2be (HIST2H2BE), |
| | | | mRNA. |
| 1260270 | AIM2 | 9447 | Homo sapiens absent in melanoma 2 (AIM2), mRNA. |
| 6400414 | LOC650546 | 650546 | PREDICTED: Homo sapiens similar to ubiquitin specific |
| | | | protease 32, transcript variant 1 (LOC650546), mRNA. |
| 4180148 | LOC650546 | 650546 | PREDICTED: Homo sapiens similar to ubiquitin specific |
| | | | protease 32, transcript variant 1 (LOC650546), mRNA. |
| 1980184 | P2RY10 | 27334 | Homo sapiens purinergic receptor P2Y, G-protein |
| | | | coupled, 10 (P2RY10), transcript variant 2, mRNA. |
| 5130021 | MECP2 | 4204 | Homo sapiens methyl CpG binding protein 2 (Rett |
| | | | syndrome) (MECP2), mRNA. |
| 6180543 | GK5 | 256356 | Homo sapiens glycerol kinase 5 (putative) (GK5), |
| | | | mRNA. |
| 3450044 | LOC100128485 | 100128485 | PREDICTED: Homo sapiens similar to conserved |
| | | | hypothetical protein (LOC100128485), mRNA. |
| 4860681 | GUCY1A3 | 2982 | Homo sapiens guanylate cyclase 1, soluble, alpha 3 |
| | | | (GUCY1A3), mRNA. |
| 2100333 | ZFP1 | 162239 | Homo sapiens zinc finger protein 1 homolog (mouse) |
| | | | (ZFP1), mRNA. |
| 6760148 | CLEC2D | 29121 | Homo sapiens C-type lectin domain family 2, member D |
| | | | (CLEC2D), transcript variant 1, mRNA. |
| 290730 | HIST1H2BD | 3017 | Homo sapiens histone cluster 1, H2bd (HIST1H2BD), |
| | | | transcript variant 2, mRNA. |
| 6200669 | HIST1H2BD | 3017 | Homo sapiens histone cluster 1, H2bd (HIST1H2BD), |
| | | | transcript variant 2, mRNA. |
| 5390427 | NAIP | 4671 | Homo sapiens NLR family, apoptosis inhibitory protein |
| | | | (NAIP), transcript variant 1, mRNA. |
| 5340240 | NAIP | 4671 | Homo sapiens NLR family, apoptosis inhibitory protein |
| | | | (NAIP), transcript variant 1, mRNA. |
| 7550358 | NELL2 | 4753 | Homo sapiens NEL-like 2 (chicken) (NELL2), mRNA. |
| 7000026 | SIDT1 | 54847 | Homo sapiens SID1 transmembrane family, member 1 |
| | | | (SIDT1), mRNA. |
| 5810136 | SLAMF1 | 6504 | Homo sapiens signaling lymphocytic activation molecule |
| | | | family member 1 (SLAMF1), mRNA. |
| 4860600 | MAPK14 | 1432 | Homo sapiens mitogen-activated protein kinase 14 |
| | | | (MAPK14), transcript variant 3, mRNA. |
| 6940402 | CUTL1 | 1523 | Homo sapiens cut-like 1, CCAAT displacement protein |
| | | | (Drosophila) (CUTL1), transcript variant 1, mRNA. |
| 2190671 | CCR3 | 1232 | Homo sapiens chemokine (C-C motif) receptor 3 (CCR3), |
| | | | transcript variant 2, mRNA. |
| 2750475 | D4S234E | 27065 | Homo sapiens DNA segment on chromosome 4 (unique) |
| | | | 234 expressed sequence (D4S234E), transcript variant 2, |
| | | | mRNA. |
| 4480053 | GCLM | 2730 | Homo sapiens glutamate-cysteine ligase, modifier subunit |
| | | | (GCLM), mRNA. |
| 6660112 | | | BX096603 Soares_testis_NHT Homo sapiens cDNA |
| | | | clone IMAGp998H151782, mRNA sequence |
| 6110753 | NBN | 4683 | Homo sapiens nibrin (NBN), transcript variant 2, mRNA. |
| 1050136 | LOC100133077 | 100133077 | PREDICTED: Homo sapiens hypothetical protein |
| | | | LOC100133077 (LOC100133077), mRNA. |
| 2810064 | BTLA | 151888 | Homo sapiens B and T lymphocyte associated (BTLA), |
| | | | mRNA. |
| 1010360 | GCH1 | 2643 | Homo sapiens GTP cyclohydrolase 1 (GCH1), transcript |
| | | | variant 3, mRNA. |
| 1260064 | LOC25845 | 25845 | Homo sapiens hypothetical LOC25845 (LOC25845), |
| | | | non-coding RNA. |
| 6620026 | CD83 | 9308 | Homo sapiens CD83 molecule (CD83), transcript variant |
| | | | 1, mRNA. |
| 1170653 | PDGFA | 5154 | Homo sapiens platelet-derived growth factor alpha |
| | | | polypeptide (PDGFA), transcript variant 2, mRNA. |
| 2230538 | LRRN3 | 54674 | Homo sapiens leucine rich repeat neuronal 3 (LRRN3), |
| | | | transcript variant 1, mRNA. |
| 5810598 | | | Homo sapiens cDNA clone MGC: 13035 |
| | | | IMAGE: 3613409, complete cds |
| 5270377 | LOC728790 | 728790 | PREDICTED: Homo sapiens hypothetical LOC728790 |
| | | | (LOC728790), mRNA. |
| 1510735 | NTN3 | 4917 | Homo sapiens netrin 3 (NTN3), mRNA. |
| 4880010 | ZBP1 | 81030 | Homo sapiens Z-DNA binding protein 1 (ZBP1), mRNA. |
| 4010270 | LOC440731 | 440731 | PREDICTED: Homo sapiens hypothetical LOC440731, |
| | | | transcript variant 2 (LOC440731), mRNA. |
| 1580360 | LOC651612 | 651612 | PREDICTED: Homo sapiens hypothetical protein |
| | | | LOC651612 (LOC651612), mRNA. |
| 60470 | STX11 | 8676 | Homo sapiens syntaxin 11 (STX11), mRNA. |
| 2060431 | LOC650261 | 650261 | PREDICTED: Homo sapiens similar to NADPH |
| | | | dependent diflavin oxidoreductase 1 (LOC650261), |
| | | | mRNA. |
| 5910019 | C1QB | 713 | Homo sapiens complement component 1, q |
| | | | subcomponent, B chain (C1QB), mRNA. |
| 6620630 | XK | 7504 | Homo sapiens X-linked Kx blood group (McLeod |
| | | | syndrome) (XK), mRNA. |
| 6580717 | ZMYND15 | 84225 | Homo sapiens zinc finger, MYND-type containing 15 |
| | | | (ZMYND15), transcript variant 2, mRNA. |
| 5360093 | SECTM1 | 6398 | Homo sapiens secreted and transmembrane 1 (SECTM1), |
| | | | mRNA. |
| 5290647 | CXCR5 | 643 | Homo sapiens chemokine (C—X—C motif) receptor 5 |
| | | | (CXCR5), transcript variant 2, mRNA. |
| 3890400 | CXCR5 | 643 | Homo sapiens chemokine (C—X—C motif) receptor 5 |
| | | | (CXCR5), transcript variant 2, mRNA. |
| 1430471 | LOC732134 | 732134 | PREDICTED: Homo sapiens similar to WHDC1L1 |
| | | | protein (LOC732134), mRNA. |
| 460438 | FAS | 355 | Homo sapiens Fas (TNF receptor superfamily, member 6) |
| | | | (FAS), transcript variant 3, mRNA. |
| 3390253 | TMEM156 | 80008 | Homo sapiens transmembrane protein 156 (TMEM156), |
| | | | mRNA. |
| 2710709 | FCGR1B | 2210 | Homo sapiens Fc fragment of IgG, high affinity Ib, |
| | | | receptor (CD64) (FCGR1B), transcript variant 1, mRNA. |
| 4040037 | EBI2 | 1880 | Homo sapiens Epstein-Barr virus induced gene 2 |
| | | | (lymphocyte-specific G protein-coupled receptor) (EBI2), |
| | | | mRNA. |
| 4490520 | EBI2 | 1880 | Homo sapiens Epstein-Barr virus induced gene 2 |
| | | | (lymphocyte-specific G protein-coupled receptor) (EBI2), |
| | | | mRNA. |
| 5390747 | SPTB | 6710 | Homo sapiens spectrin, beta, erythrocytic (SPTB), |
| | | | transcript variant 2, mRNA. |
| 3940056 | AIG1 | 51390 | Homo sapiens androgen-induced 1 (AIG1), mRNA. |
| 1190167 | LOC152195 | 152195 | Homo sapiens hypothetical protein LOC152195 |
| | | | (LOC152195), mRNA. |
| 3800050 | ADCY3 | 109 | Homo sapiens adenylate cyclase 3 (ADCY3), mRNA. |
| 6450154 | LSS | 4047 | Homo sapiens lanosterol synthase (2,3-oxidosqualene- |
| | | | lanosterol cyclase) (LSS), transcript variant 1, mRNA. |
| 830440 | TLR5 | 7100 | Homo sapiens toll-like receptor 5 (TLR5), mRNA. |
| 3140386 | NFATC3 | 4775 | Homo sapiens nuclear factor of activated T-cells, |
| | | | cytoplasmic, calcineurin-dependent 3 (NFATC3), |
| | | | transcript variant 3, mRNA. |
| 3060523 | NAMPT | 10135 | Homo sapiens nicotinamide phosphoribosyltransferase |
| | | | (NAMPT), mRNA. |
| 2230402 | TTC35 | 9694 | Homo sapiens tetratricopeptide repeat domain 35 |
| | | | (TTC35), mRNA. |
| 1260482 | GZMK | 3003 | Homo sapiens granzyme K (granzyme 3; tryptase II) |
| | | | (GZMK), mRNA. |
| 5560270 | CARD17 | 440068 | Homo sapiens caspase recruitment domain family, |
| | | | member 17 (CARD17), mRNA. |
| 3930368 | CARD17 | 440068 | Homo sapiens caspase recruitment domain family, |
| | | | member 17 (CARD17), mRNA. |
| 7320731 | MAP3K4 | 4216 | Homo sapiens mitogen-activated protein kinase kinase |
| | | | kinase 4 (MAP3K4), transcript variant 1, mRNA. |
| 4230735 | FAM129A | 116496 | Homo sapiens family with sequence similarity 129, |
| | | | member A (FAM129A), transcript variant 2, mRNA. |
| 2970408 | CD8A | 925 | Homo sapiens CD8a molecule (CD8A), transcript variant |
| | | | 1, mRNA. |
| 2120056 | | | oj15g10.y1 Human lacrimal gland, unamplified: of Homo |
| | | | sapiens cDNA clone oj15g10 5, mRNA sequence |
| 870349 | ACBD6 | 84320 | Homo sapiens acyl-Coenzyme A binding domain |
| | | | containing 6 (ACBD6), mRNA. |
| 7160750 | MIIP | 60672 | Homo sapiens migration and invasion inhibitory protein |
| | | | (MIIP), mRNA. |
| 6480064 | POLR1E | 64425 | Homo sapiens polymerase (RNA) I polypeptide E, 53 kDa |
| | | | (POLR1E), mRNA. |
| 5050112 | C10orf119 | 79892 | Homo sapiens chromosome 10 open reading frame 119 |
| | | | (C10orf119), mRNA. |
| 4290056 | C17orf60 | 284021 | Homo sapiens chromosome 17 open reading frame 60 |
| | | | (C17orf60), mRNA. |
| 4250722 | LOC729780 | 729780 | PREDICTED: Homo sapiens misc_RNA (LOC729780), |
| | | | miscRNA. |
| 3190053 | LOC642816 | 642816 | PREDICTED: Homo sapiens similar to hypothetical |
| | | | protein LOC284701, transcript variant 1 (LOC642816), |
| | | | mRNA. |
| 360132 | LHFPL2 | 10184 | Homo sapiens lipoma HMGIC fusion partner-like 2 |
| | | | (LHFPL2), mRNA. |
| 1050025 | CR1 | 1378 | Homo sapiens complement component (3b/4b) receptor 1 |
| | | | (Knops blood group) (CR1), transcript variant F, mRNA. |
| 3990435 | | | BX282075 NIH_MGC_120 Homo sapiens cDNA clone |
| | | | IMAGp998M2011561; IMAGE: 5223355, mRNA |
| | | | sequence |
| 4640184 | KIAA1026 | 23254 | Homo sapiens kazrin (KIAA1026), transcript variant B, |
| | | | mRNA. |
| 3840215 | FAM102A | 399665 | Homo sapiens family with sequence similarity 102, |
| | | | member A (FAM102A), transcript variant 1, mRNA. |
| 1850546 | FAM102A | 399665 | Homo sapiens family with sequence similarity 102, |
| | | | member A (FAM102A), transcript variant 1, mRNA. |
| 6350240 | SMYD4 | 114826 | Homo sapiens SET and MYND domain containing 4 |
| | | | (SMYD4), mRNA. |
| 60202 | ELAC2 | 60528 | Homo sapiens elaC homolog 2 (E. coli) (ELAC2), |
| | | | mRNA. |
| 3440164 | LOC642469 | 642469 | PREDICTED: Homo sapiens misc_RNA (LOC642469), |
| | | | miscRNA. |
| 1090280 | ZNF680 | 340252 | Homo sapiens zinc finger protein 680 (ZNF680), mRNA. |
| 4220592 | CACNA2D3 | 55799 | Homo sapiens calcium channel, voltage-dependent, alpha |
| | | | 2/delta subunit 3 (CACNA2D3), mRNA. |
| 7610494 | CDCA4 | 55038 | Homo sapiens cell division cycle associated 4 (CDCA4), |
| | | | transcript variant 13, mRNA. |
| 6290754 | LOC652234 | 652234 | PREDICTED: Homo sapiens similar to stereocilin |
| | | | (LOC652234), mRNA. |
| 4070538 | TDP1 | 55775 | Homo sapiens tyrosyl-DNA phosphodiesterase 1 (TDP1), |
| | | | transcript variant 1, mRNA. |
| 5390497 | C7orf53 | 286006 | Homo sapiens chromosome 7 open reading frame 53 |
| | | | (C7orf53), mRNA. |
| 5870148 | CHD1L | 9557 | Homo sapiens chromodomain helicase DNA binding |
| | | | protein 1-like (CHD1L), mRNA. |
| 2120537 | SPOCD1 | 90853 | Homo sapiens SPOC domain containing 1 (SPOCD1), |
| | | | mRNA. |
| 6450326 | CHMP5 | 51510 | Homo sapiens chromatin modifying protein 5 (CHMP5), |
| | | | mRNA. |
| 6280138 | PPM1B | 5495 | Homo sapiens protein phosphatase 1B (formerly 2C), |
| | | | magnesium-dependent, beta isoform (PPM1B), transcript |
| | | | variant 4, mRNA. |
| 4640427 | HIATL1 | 84641 | Homo sapiens hippocampus abundant transcript-like 1 |
| | | | (HIATL1), mRNA. |
| 6420594 | TIFA | 92610 | Homo sapiens TRAF-interacting protein with forkhead- |
| | | | associated domain (TIFA), mRNA. |
| 1300192 | ARL4C | 10123 | Homo sapiens ADP-ribosylation factor-like 4C (ARL4C), |
| | | | mRNA. |
| 70110 | TCL1A | 8115 | Homo sapiens T-cell leukemia/lymphoma 1A (TCL1A), |
| | | | transcript variant 2, mRNA. |
| 5220767 | FLNB | 2317 | Homo sapiens filamin B, beta (actin binding protein 278) |
| | | | (FLNB), mRNA. |
| 6650482 | EPHA4 | 2043 | Homo sapiens EPH receptor A4 (EPHA4), mRNA. |
| 4390070 | EIF1AY | 9086 | Homo sapiens eukaryotic translation initiation factor 1A, |
| | | | Y-linked (EIF1AY), mRNA. |
| 1340670 | KIR2DL3 | 3804 | Homo sapiens killer cell immunoglobulin-like receptor, |
| | | | two domains, long cytoplasmic tail, 3 (KIR2DL3), |
| | | | transcript variant 1, mRNA. |
| 1070367 | C19orf59 | 199675 | Homo sapiens chromosome 19 open reading frame 59 |
| | | | (C19orf59), mRNA. |
| 1990019 | PPP2R2B | 5521 | Homo sapiens protein phosphatase 2 (formerly 2A), |
| | | | regulatory subunit B, beta isoform (PPP2R2B), transcript |
| | | | variant 4, mRNA. |
| 6020523 | PPP2R2B | 5521 | Homo sapiens protein phosphatase 2 (formerly 2A), |
| | | | regulatory subunit B, beta isoform (PPP2R2B), transcript |
| | | | variant 4, mRNA. |
| 4260017 | CDK5RAP2 | 55755 | Homo sapiens CDK5 regulatory subunit associated |
| | | | protein 2 (CDK5RAP2), transcript variant 2, mRNA. |
| 3890609 | PLSCR1 | 5359 | Homo sapiens phospholipid scramblase 1 (PLSCR1), |
| | | | mRNA. |
| 1050292 | GP9 | 2815 | Homo sapiens glycoprotein IX (platelet) (GP9), mRNA. |
| 6480630 | ATP9A | 10079 | Homo sapiens ATPase, class II, type 9A (ATP9A), |
| | | | mRNA. |
| 5360326 | SIP1 | 8487 | Homo sapiens survival of motor neuron protein |
| | | | interacting protein 1 (SIP1), transcript variant beta, |
| | | | mRNA. |
| 4390349 | ZNF256 | 10172 | Homo sapiens zinc finger protein 256 (ZNF256), mRNA. |
| 870202 | TNFSF10 | 8743 | Homo sapiens tumor necrosis factor (ligand) superfamily, |
| | | | member 10 (TNFSF10), mRNA. |
| 3780382 | DKFZp761E198 | 91056 | Homo sapiens DKFZp761E198 protein |
| | | | (DKFZp761E198), mRNA. |
| 5560280 | SAMD14 | 201191 | Homo sapiens sterile alpha motif domain containing 14 |
| | | | (SAMD14), mRNA. |
| 4150692 | PARP14 | 54625 | Homo sapiens poly (ADP-ribose) polymerase family, |
| | | | member 14 (PARP14), mRNA. |
| 5220612 | ECHDC3 | 79746 | Homo sapiens enoyl Coenzyme A hydratase domain |
| | | | containing 3 (ECHDC3), mRNA. |
| 3310025 | OCIAD2 | 132299 | Homo sapiens OCIA domain containing 2 (OCIAD2), |
| | | | transcript variant 2, mRNA. |
| 4670113 | LOC90925 | 90925 | Homo sapiens hypothetical protein LOC90925 |
| | | | (LOC90925), mRNA. |
| 3180209 | ANK1 | 286 | Homo sapiens ankyrin 1, erythrocytic (ANK1), transcript |
| | | | variant 5, mRNA. |
| 5700735 | PARP9 | 83666 | Homo sapiens poly (ADP-ribose) polymerase family, |
| | | | member 9 (PARP9), mRNA. |
| 3140707 | PARP9 | 83666 | Homo sapiens poly (ADP-ribose) polymerase family, |
| | | | member 9 (PARP9), mRNA. |
| 1940132 | CD151 | 977 | Homo sapiens CD151 molecule (Raph blood group) |
| | | | (CD151), transcript variant 4, mRNA. |
| 6650064 | SIGLEC5 | 8778 | Homo sapiens sialic acid binding Ig-like lectin 5 |
| | | | (SIGLEC5), mRNA. |
| 4640270 | SIGLEC5 | 8778 | Homo sapiens sialic acid binding Ig-like lectin 5 |
| | | | (SIGLEC5), mRNA. |
| 5290292 | DHRS9 | 10170 | Homo sapiens dehydrogenase/reductase (SDR family) |
| | | | member 9 (DHRS9), transcript variant 2, mRNA. |
| 5090408 | LRIG1 | 26018 | Homo sapiens leucine-rich repeats and immunoglobulin- |
| | | | like domains 1 (LRIG1), mRNA. |
| 990239 | KCNE3 | 10008 | Homo sapiens potassium voltage-gated channel, Isk- |
| | | | related family, member 3 (KCNE3), mRNA. |
| 6100064 | CRYGS | 1427 | Homo sapiens crystallin, gamma S (CRYGS), mRNA. |
| 6940274 | PTGDR | 5729 | Homo sapiens prostaglandin D2 receptor (DP) (PTGDR), |
| | | | mRNA. |
| 2810471 | PTGDR | 5729 | Homo sapiens prostaglandin D2 receptor (DP) (PTGDR), |
| | | | mRNA. |
| 1030431 | ACSL1 | 2180 | Homo sapiens acyl-CoA synthetase long-chain family |
| | | | member 1 (ACSL1), mRNA. |
| 3360132 | SNX20 | 124460 | Homo sapiens sorting nexin 20 (SNX20), mRNA. |
| 1050068 | F2RL1 | 2150 | Homo sapiens coagulation factor II (thrombin) receptor- |
| | | | like 1 (F2RL1), mRNA. |
| 5810047 | F2RL1 | 2150 | Homo sapiens coagulation factor II (thrombin) receptor- |
| | | | like 1 (F2RL1), mRNA. |
| 5560075 | MFGE8 | 4240 | Homo sapiens milk fat globule-EGF factor 8 protein |
| | | | (MFGE8), mRNA. |
| 540025 | IL4R | 3566 | Homo sapiens interleukin 4 receptor (IL4R), transcript |
| | | | variant 2, mRNA. |
| 7570600 | FLJ33590 | 285093 | Homo sapiens hypothetical protein FLJ33590 |
| | | | (FLJ33590), mRNA. |
| 2470601 | IL1RN | 3557 | Homo sapiens interleukin 1 receptor antagonist (IL1RN), |
| | | | transcript variant 1, mRNA. |
| 3120474 | KIAA1671 | 85379 | PREDICTED: Homo sapiens KIAA1671 protein |
| | | | (KIAA1671), mRNA. |
| 990768 | OAS3 | 4940 | Homo sapiens 2′-5′-oligoadenylate synthetase 3, 100 kDa |
| | | | (OAS3), mRNA. |
| 830176 | C2orf18 | 54978 | Homo sapiens chromosome 2 open reading frame 18 |
| | | | (C2orf18), mRNA. |
| 6900521 | PHTF1 | 10745 | Homo sapiens putative homeodomain transcription factor |
| | | | 1 (PHTF1), mRNA. |
| 3190521 | MS4A1 | 931 | Homo sapiens membrane-spanning 4-domains, subfamily |
| | | | A, member 1 (MS4A1), transcript variant 1, mRNA. |
| 60719 | KCNJ2 | 3759 | Homo sapiens potassium inwardly-rectifying channel, |
| | | | subfamily J, member 2 (KCNJ2), mRNA. |
| 7400259 | IDUA | 3425 | Homo sapiens iduronidase, alpha-L-(IDUA), mRNA. |
| 5340767 | CEACAM1 | 634 | Homo sapiens carcinoembryonic antigen-related cell |
| | | | adhesion molecule 1 (biliary glycoprotein) (CEACAM1), |
| | | | transcript variant 1, mRNA. |
| 2100725 | KIR3DL3 | 115653 | Homo sapiens killer cell immunoglobulin-like receptor, |
| | | | three domains, long cytoplasmic tail, 3 (KIR3DL3), |
| | | | mRNA. |
| 3370577 | APP | 351 | Homo sapiens amyloid beta (A4) precursor protein |
| | | | (APP), transcript variant 3, mRNA. |
| 650110 | APP | 351 | Homo sapiens amyloid beta (A4) precursor protein |
| | | | (APP), transcript variant 3, mRNA. |
| 5220041 | HSZFP36 | 55552 | Homo sapiens ZFP-36 for a zinc finger protein |
| | | | (HSZFP36), mRNA. |
| 3930020 | CTSF | 8722 | Homo sapiens cathepsin F (CTSF), mRNA. |
| 450189 | XAF1 | 54739 | Homo sapiens XIAP associated factor 1 (XAF1), |
| | | | transcript variant 2, mRNA. |
| 7610440 | XAF1 | 54739 | Homo sapiens XIAP associated factor 1 (XAF1), |
| | | | transcript variant 2, mRNA. |
| 2810692 | KIAA1881 | 114782 | PREDICTED: Homo sapiens KIAA1881 (KIAA1881), |
| | | | mRNA. |
| 1940373 | ZNF831 | 128611 | Homo sapiens zinc finger protein 831 (ZNF831), mRNA. |
| 6270040 | ZNF831 | 128611 | Homo sapiens zinc finger protein 831 (ZNF831), mRNA. |
| 2710646 | MSRB2 | 22921 | Homo sapiens methionine sulfoxide reductase B2 |
| | | | (MSRB2), mRNA. |
| 110372 | CSTA | 1475 | Homo sapiens cystatin A (stefin A) (CSTA), mRNA. |
| 3440376 | FECH | 2235 | Homo sapiens ferrochelatase (protoporphyria) (FECH), |
| | | | nuclear gene encoding mitochondrial protein, transcript |
| | | | variant 1, mRNA. |
| 6380411 | IKZF3 | 22806 | Homo sapiens IKAROS family zinc finger 3 (Aiolos) |
| | | | (IKZF3), transcript variant 1, mRNA. |
| 380050 | RHBDF2 | 79651 | Homo sapiens rhomboid 5 homolog 2 (Drosophila) |
| | | | (RHBDF2), transcript variant 1, mRNA. |
| 6200019 | KLRB1 | 3820 | Homo sapiens killer cell lectin-like receptor subfamily B, |
| | | | member 1 (KLRB1), mRNA. |
| 1740022 | SPNS3 | 201305 | Homo sapiens spinster homolog 3 (Drosophila) (SPNS3), |
| | | | mRNA. |
| 4810768 | S1PR1 | 1901 | Homo sapiens sphingosine-1-phosphate receptor 1 |
| | | | (S1PR1), mRNA. |
| 3310341 | FYB | 2533 | Homo sapiens FYN binding protein (FYB-120/130) |
| | | | (FYB), transcript variant 1, mRNA. |
| 4010634 | CRYBB2 | 1415 | Homo sapiens crystallin, beta B2 (CRYBB2), mRNA. |
| 1070470 | | | Homo sapiens cDNA FLJ34018 fis, clone |
| | | | FCBBF2002801 |
| 4260725 | CD79B | 974 | Homo sapiens CD79B antigen (immunoglobulin- |
| | | | associated beta) (CD79B), transcript variant 2, mRNA. |
| 3780619 | TMEM14A | 28978 | Homo sapiens transmembrane protein 14A (TMEM14A), |
| | | | mRNA. |
| 60014 | FAM84B | 157638 | Homo sapiens family with sequence similarity 84, |
| | | | member B (FAM84B), mRNA. |
| 990470 | LOC651738 | 651738 | PREDICTED: Homo sapiens hypothetical LOC651738 |
| | | | (LOC651738), mRNA. |
| 670603 | PLAGL1 | 5325 | Homo sapiens pleiomorphic adenoma gene-like 1 |
| | | | (PLAGL1), transcript variant 3, mRNA. |
| 3310376 | TIMM10 | 26519 | Homo sapiens translocase of inner mitochondrial |
| | | | membrane 10 homolog (yeast) (TIMM10), nuclear gene |
| | | | encoding mitochondrial protein, mRNA. |
| 150626 | LOC641710 | 641710 | PREDICTED: Homo sapiens hypothetical protein |
| | | | LOC641710, transcript variant 1 (LOC641710), mRNA. |
| 7610598 | TRAF5 | 7188 | Homo sapiens TNF receptor-associated factor 5 |
| | | | (TRAF5), transcript variant 1, mRNA. |
| 7330392 | TAP1 | 6890 | Homo sapiens transporter 1, ATP-binding cassette, sub- |
| | | | family B (MDR/TAP) (TAP1), mRNA. |
| 6770070 | FCRL2 | 79368 | Homo sapiens Fc receptor-like 2 (FCRL2), transcript |
| | | | variant 2, mRNA. |
| 7000474 | RALGAPA1 | 253959 | Homo sapiens Ral GTPase activating protein, alpha |
| | | | subunit 1 (catalytic) (RALGAPA1), transcript variant 2, |
| | | | mRNA. |
| 7400025 | MTP18 | 51537 | Homo sapiens mitochondrial protein 18 kDa (MTP18), |
| | | | nuclear gene encoding mitochondrial protein, transcript |
| | | | variant 1, mRNA. |
| 3140064 | C22orf34 | 348645 | Homo sapiens chromosome 22 open reading frame 34 |
| | | | (C22orf34), mRNA. |
| 4610543 | TLE1 | 7088 | Homo sapiens transducin-like enhancer of split 1 (E(sp1) |
| | | | homolog, Drosophila) (TLE1), mRNA. |
| 1010170 | BACH1 | 571 | Homo sapiens BTB and CNC homology 1, basic leucine |
| | | | zipper transcription factor 1 (BACH1), transcript variant |
| | | | 3, mRNA. |
| 4120309 | C7orf36 | 57002 | Homo sapiens chromosome 7 open reading frame 36 |
| | | | (C7orf36), mRNA. |
| 6280301 | LOC643313 | 643313 | PREDICTED: Homo sapiens similar to hypothetical |
| | | | protein LOC284701, transcript variant 3 (LOC643313), |
| | | | mRNA. |
| 1450112 | REPS2 | 9185 | Homo sapiens RALBP1 associated Eps domain |
| | | | containing 2 (REPS2), transcript variant 2, mRNA. |
| 1570358 | APOL2 | 23780 | Homo sapiens apolipoprotein L, 2 (APOL2), transcript |
| | | | variant beta, mRNA. |
| 3370066 | APOL2 | 23780 | Homo sapiens apolipoprotein L, 2 (APOL2), transcript |
| | | | variant beta, mRNA. |
| 2140382 | PASK | 23178 | Homo sapiens PAS domain containing serine/threonine |
| | | | kinase (PASK), mRNA. |
| 4810021 | SPINT3 | 10816 | Homo sapiens serine peptidase inhibitor, Kunitz type, 3 |
| | | | (SPINT3), mRNA. |
| 6510707 | FER1L3 | 26509 | Homo sapiens fer-1-like 3, myoferlin (C. elegans) |
| | | | (FER1L3), transcript variant 1, mRNA. |
| 2710255 | HNRNPUL1 | 11100 | Homo sapiens heterogeneous nuclear ribonucleoprotein |
| | | | U-like 1 (HNRNPUL1), transcript variant 4, mRNA. |
| 4060088 | LOC644677 | 644677 | PREDICTED: Homo sapiens hypothetical protein |
| | | | LOC644677 (LOC644677), mRNA. |
| 3800019 | NCR3 | 259197 | Homo sapiens natural cytotoxicity triggering receptor 3 |
| | | | (NCR3), mRNA. |
| 4210753 | CD8B | 926 | Homo sapiens CD8b molecule (CD8B), transcript variant |
| | | | 5, mRNA. |
| 3830433 | | | BX096301 Soares retina N2b5HR Homo sapiens cDNA |
| | | | clone IMAGp998C16444, mRNA sequence |
| 1710553 | HSPA6 | 3310 | Homo sapiens heat shock 70 kDa protein 6 (HSP70B′) |
| | | | (HSPA6), mRNA. |
| 2940685 | C1QA | 712 | Homo sapiens complement component 1, q |
| | | | subcomponent, alpha polypeptide (C1QA), mRNA. |
| 3870133 | LOC100133177 | 100133177 | PREDICTED: Homo sapiens misc_RNA |
| | | | (LOC100133177), miscRNA. |
| 2230553 | USP32 | 84669 | Homo sapiens ubiquitin specific peptidase 32 (USP32), |
| | | | mRNA. |
| 5290369 | USP32 | 84669 | Homo sapiens ubiquitin specific peptidase 32 (USP32), |
| | | | mRNA. |
| 4150543 | FPR2 | 2358 | Homo sapiens formyl peptide receptor 2 (FPR2), |
| | | | transcript variant 2, mRNA. |
| 5670095 | ZNF239 | 8187 | Homo sapiens zinc finger protein 239 (ZNF239), |
| | | | transcript variant 4, mRNA. |
| 7330671 | APOBEC3A | 200315 | Homo sapiens apolipoprotein B mRNA editing enzyme, |
| | | | catalytic polypeptide-like 3A (APOBEC3A), mRNA. |
| 6580639 | ACOT7 | 11332 | Homo sapiens acyl-CoA thioesterase 7 (ACOT7), |
| | | | transcript variant hBACHa, mRNA. |
| 2030678 | HIST2H2AB | 317772 | Homo sapiens histone cluster 2, H2ab (HIST2H2AB), |
| | | | mRNA. |
| 770253 | KIAA1618 | 57714 | PREDICTED: Homo sapiens KIAA1618 (KIAA1618), |
| | | | mRNA. |
| 2000292 | SCGB3A1 | 92304 | Homo sapiens secretoglobin, family 3A, member 1 |
| | | | (SCGB3A1), mRNA. |
| 2680189 | LAG3 | 3902 | Homo sapiens lymphocyte-activation gene 3 (LAG3), |
| | | | mRNA. |
| 50136 | CMTM5 | 116173 | Homo sapiens CKLF-like MARVEL transmembrane |
| | | | domain containing 5 (CMTM5), transcript variant 3, |
| | | | mRNA. |
| 3400164 | C21orf2 | 755 | Homo sapiens chromosome 21 open reading frame 2 |
| | | | (C21orf2), mRNA. |
| 5050138 | NT5M | 56953 | Homo sapiens 5′,3′-nucleotidase, mitochondrial (NT5M), |
| | | | nuclear gene encoding mitochondrial protein, mRNA. |
| 2600739 | VRK1 | 7443 | Homo sapiens vaccinia related kinase 1 (VRK1), mRNA. |
| 4290619 | CREB5 | 9586 | Homo sapiens cAMP responsive element binding protein |
| | | | 5 (CREB5), transcript variant 4, mRNA. |
| 1010154 | LOC643008 | 643008 | PREDICTED: Homo sapiens PP12104, transcript variant |
| | | | 1 (LOC643008), mRNA. |
| 2640603 | CLEC7A | 64581 | Homo sapiens C-type lectin domain family 7, member A |
| | | | (CLEC7A), transcript variant 5, mRNA. |
| 3420026 | FAS | 355 | Homo sapiens Fas (TNF receptor superfamily, member 6) |
| | | | (FAS), transcript variant 7, mRNA. |
| 2030209 | MTF1 | 4520 | Homo sapiens metal-regulatory transcription factor 1 |
| | | | (MTF1), mRNA. |
| 1260192 | QKI | 9444 | Homo sapiens quaking homolog, KH domain RNA |
| | | | binding (mouse) (QKI), transcript variant 2, mRNA. |
| 520010 | LOC284701 | 284701 | PREDICTED: Homo sapiens hypothetical protein |
| | | | LOC284701, transcript variant 2 (LOC284701), mRNA. |
| 3360343 | RSAD2 | 91543 | Homo sapiens radical S-adenosyl methionine domain |
| | | | containing 2 (RSAD2), mRNA. |
| 7160066 | C20orf196 | 149840 | Homo sapiens chromosome 20 open reading frame 196 |
| | | | (C20orf196), mRNA. |
| 2760307 | | | DB113199 THYMU2 Homo sapiens cDNA clone |
| | | | THYMU2017679 5, mRNA sequence |
| 4570746 | TAF1 | 6872 | Homo sapiens TAF1 RNA polymerase II, TATA box |
| | | | binding protein (TBP)-associated factor, 250 kDa (TAF1), |
| | | | transcript variant 2, mRNA. |
| 7050382 | CASP1 | 834 | Homo sapiens caspase 1, apoptosis-related cysteine |
| | | | peptidase (interleukin 1, beta, convertase) (CASP1), |
| | | | transcript variant delta, mRNA. |
| 2140678 | ITGA6 | 3655 | Homo sapiens integrin, alpha 6 (ITGA6), transcript |
| | | | variant 2, mRNA. |
| 5310437 | MYL9 | 10398 | Homo sapiens myosin, light chain 9, regulatory (MYL9), |
| | | | transcript variant 1, mRNA. |
| 2480327 | MYL9 | 10398 | Homo sapiens myosin, light chain 9, regulatory (MYL9), |
| | | | transcript variant 1, mRNA. |
| 1580750 | LOC100130828 | 100130828 | PREDICTED: Homo sapiens hypothetical |
| | | | LOC100130828 (LOC100130828), mRNA. |
| 5340468 | ITGA2B | 3674 | Homo sapiens integrin, alpha 2b (platelet glycoprotein IIb |
| | | | of IIb/IIIa complex, antigen CD41B) (ITGA2B), mRNA. |
| 1010246 | IFI6 | 2537 | Homo sapiens interferon, alpha-inducible protein 6 |
| | | | (IFI6), transcript variant 2, mRNA. |
| 1090390 | OAS1 | 4938 | Homo sapiens 2′,5′-oligoadenylate synthetase 1, |
| | | | 40/46 kDa (OAS1), transcript variant 3, mRNA. |
| 7040035 | OAS1 | 4938 | Homo sapiens 2′,5′-oligoadenylate synthetase 1, |
| | | | 40/46 kDa (OAS1), transcript variant 3, mRNA. |
| 940129 | WDR21A | 26094 | Homo sapiens WD repeat domain 21A (WDR21A), |
| | | | transcript variant 1, mRNA. |
| 2140020 | LIMK2 | 3985 | Homo sapiens LIM domain kinase 2 (LIMK2), transcript |
| | | | variant 2b, mRNA. |
| 3390048 | LIMK2 | 3985 | Homo sapiens LIM domain kinase 2 (LIMK2), transcript |
| | | | variant 2b, mRNA. |
| 5910632 | SMARCD3 | 6604 | Homo sapiens SWI/SNF related, matrix associated, actin |
| | | | dependent regulator of chromatin, subfamily d, member 3 |
| | | | (SMARCD3), transcript variant 2, mRNA. |
| 3420008 | SNX6 | 58533 | Homo sapiens sorting nexin 6 (SNX6), transcript variant |
| | | | 1, mRNA. |
| 5570180 | LCK | 3932 | Homo sapiens lymphocyte-specific protein tyrosine |
| | | | kinase (LCK), mRNA. |
| 3170136 | SAMD9L | 219285 | Homo sapiens sterile alpha motif domain containing 9- |
| | | | like (SAMD9L), mRNA. |
| 5260349 | NGFRAP1 | 27018 | Homo sapiens nerve growth factor receptor (TNFRSF16) |
| | | | associated protein 1 (NGFRAP1), transcript variant 1, |
| | | | mRNA. |
| 6860220 | NGFRAP1 | 27018 | Homo sapiens nerve growth factor receptor (TNFRSF16) |
| | | | associated protein 1 (NGFRAP1), transcript variant 1, |
| | | | mRNA. |
| 3610707 | GCOM1 | 145781 | Homo sapiens GRINL1A complex locus (GCOM1), |
| | | | transcript variant 2, mRNA. |
| 7400377 | CEACAM6 | 4680 | Homo sapiens carcinoembryonic antigen-related cell |
| | | | adhesion molecule 6 (non-specific cross reacting antigen) |
| | | | (CEACAM6), mRNA. |
| 5860722 | MIR155HG | 114614 | Homo sapiens MIR155 host gene (non-protein coding) |
| | | | (MIR155HG), non-coding RNA. |
| 4760601 | LOC728014 | 728014 | PREDICTED: Homo sapiens similar to huntingtin |
| | | | interacting protein 1 related (LOC728014), mRNA. |
| 3940041 | SIRPG | 55423 | Homo sapiens signal-regulatory protein gamma (SIRPG), |
| | | | transcript variant 3, mRNA. |
| 5050537 | OPLAH | 26873 | Homo sapiens 5-oxoprolinase (ATP-hydrolysing) |
| | | | (OPLAH), mRNA. |
| 4060561 | FTHL2 | 2497 | Homo sapiens ferritin, heavy polypeptide-like 2 (FTHL2) |
| | | | on chromosome 1. |
| 4900747 | C11orf75 | 56935 | Homo sapiens chromosome 11 open reading frame 75 |
| | | | (C11orf75), mRNA. |
| 2030148 | WDR54 | 84058 | Homo sapiens WD repeat domain 54 (WDR54), mRNA. |
| 6400332 | CNIH4 | 29097 | Homo sapiens cornichon homolog 4 (Drosophila) |
| | | | (CNIH4), mRNA. |
| 3170703 | LY9 | 4063 | Homo sapiens lymphocyte antigen 9 (LY9), transcript |
| | | | variant 2, mRNA. |
| 1240358 | RAB20 | 55647 | Homo sapiens RAB20, member RAS oncogene family |
| | | | (RAB20), mRNA. |
| 450647 | LOC728743 | 728743 | PREDICTED: Homo sapiens misc_RNA (LOC728743), |
| | | | miscRNA. |
| 1990300 | SOCS1 | 8651 | Homo sapiens suppressor of cytokine signaling 1 |
| | | | (SOCS1), mRNA. |
| 5550743 | LOC730051 | 730051 | PREDICTED: Homo sapiens similar to Zinc finger |
| | | | protein 418 (LOC730051), mRNA. |
| 670288 | ZNF571 | 51276 | Homo sapiens zinc finger protein 571 (ZNF571), mRNA. |
| 460064 | DGKA | 1606 | Homo sapiens diacylglycerol kinase, alpha 80 kDa |
| | | | (DGKA), transcript variant 3, mRNA. |
| 7040477 | PLOD2 | 5352 | Homo sapiens procollagen-lysine, 2-oxoglutarate 5- |
| | | | dioxygenase 2 (PLOD2), transcript variant 2, mRNA. |
| 7200338 | BMP2K | 55589 | Homo sapiens BMP2 inducible kinase (BMP2K), |
| | | | transcript variant 1, mRNA. |
| 1470685 | ITGB3 | 3690 | Homo sapiens integrin, beta 3 (platelet glycoprotein IIIa, |
| | | | antigen CD61) (ITGB3), mRNA. |
| 4780477 | LOC100133329 | 100133329 | PREDICTED: Homo sapiens misc_RNA |
| | | | (LOC100133329), miscRNA. |
| 4050156 | ERBB2 | 2064 | Homo sapiens v-erb-b2 erythroblastic leukemia viral |
| | | | oncogene homolog 2, neuro/glioblastoma derived |
| | | | oncogene homolog (avian) (ERBB2), transcript variant 1, |
| | | | mRNA. |
| 630315 | DHRS9 | 10170 | Homo sapiens dehydrogenase/reductase (SDR family) |
| | | | member 9 (DHRS9), transcript variant 1, mRNA. |
| 3780647 | KIAA0125 | 9834 | Homo sapiens KIAA0125 (KIAA0125), mRNA. |
| 1070209 | ASAP1IT1 | 29065 | Homo sapiens ASAP1 intronic transcript 1 (non-protein |
| | | | coding) (ASAP1IT1), non-coding RNA. |
| 1660021 | RNU4ATAC | 100151683 | Homo sapiens RNA, U4atac small nuclear (U12- |
| | | | dependent splicing) (RNU4ATAC), small nuclear RNA. |
| 3800707 | XPO4 | 64328 | Homo sapiens exportin 4 (XPO4), mRNA. |
| 5090035 | PPP2R5D | 5528 | Homo sapiens protein phosphatase 2, regulatory subunit |
| | | | B′, delta isoform (PPP2R5D), transcript variant 2, mRNA. |
| 3940309 | CDAN1 | 146059 | PREDICTED: Homo sapiens congenital |
| | | | dyserythropoietic anemia, type I (CDAN1), mRNA. |
| 620167 | FCRL3 | 115352 | Homo sapiens Fc receptor-like 3 (FCRL3), mRNA. |
| 5420373 | FUT6 | 2528 | Homo sapiens fucosyltransferase 6 (alpha (1,3) |
| | | | fucosyltransferase) (FUT6), transcript variant 1, mRNA. |
| 540382 | RSAD1 | 55316 | Homo sapiens radical S-adenosyl methionine domain |
| | | | containing 1 (RSAD1), mRNA. |
| 2100196 | ISG15 | 9636 | Homo sapiens ISG15 ubiquitin-like modifier (ISG15), |
| | | | mRNA. |
| 6760286 | | | RST24587 Athersys RAGE Library Homo sapiens |
| | | | cDNA, mRNA sequence |
| 4290148 | HIST2H2AA4 | 723790 | Homo sapiens histone cluster 2, H2aa4 (HIST2H2AA4), |
| | | | mRNA. |
| 1780440 | CD79A | 973 | Homo sapiens CD79a molecule, immunoglobulin- |
| | | | associated alpha (CD79A), transcript variant 1, mRNA. |
| 3130220 | TMEM158 | 25907 | Homo sapiens transmembrane protein 158 (TMEM158), |
| | | | mRNA. |
| 2690240 | FANCD2 | 2177 | Homo sapiens Fanconi anemia, complementation group |
| | | | D2 (FANCD2), transcript variant 2, mRNA. |
| 4150278 | DUSP14 | 11072 | Homo sapiens dual specificity phosphatase 14 (DUSP14), |
| | | | mRNA. |
| 1260767 | FAM3C | 10447 | Homo sapiens family with sequence similarity 3, member |
| | | | C (FAM3C), transcript variant 2, mRNA. |
| 3290292 | LAP3 | 51056 | Homo sapiens leucine aminopeptidase 3 (LAP3), mRNA. |
| 830341 | TBRG4 | 9238 | Homo sapiens transforming growth factor beta regulator 4 |
| | | | (TBRG4), transcript variant 2, mRNA. |
| 3190608 | SLC6A9 | 6536 | Homo sapiens solute carrier family 6 (neurotransmitter |
| | | | transporter, glycine), member 9 (SLC6A9), transcript |
| | | | variant 3, mRNA. |
| 4900435 | TNFSF13B | 10673 | Homo sapiens tumor necrosis factor (ligand) superfamily, |
| | | | member 13b (TNFSF13B), transcript variant 1, mRNA. |
| 1240553 | SESN1 | 27244 | Homo sapiens sestrin 1 (SESN1), mRNA. |
| 4850592 | P2RY14 | 9934 | Homo sapiens purinergic receptor P2Y, G-protein |
| | | | coupled, 14 (P2RY14), transcript variant 2, mRNA. |
| 4610241 | P2RY14 | 9934 | Homo sapiens purinergic receptor P2Y, G-protein |
| | | | coupled, 14 (P2RY14), transcript variant 2, mRNA. |
| 1340364 | P2RY14 | 9934 | Homo sapiens purinergic receptor P2Y, G-protein |
| | | | coupled, 14 (P2RY14), transcript variant 2, mRNA. |
| 6560072 | | | EST366269 MAGE resequences, MAGC Homo sapiens |
| | | | cDNA, mRNA sequence |
| 4220152 | SIRPG | 55423 | Homo sapiens signal-regulatory protein gamma (SIRPG), |
| | | | transcript variant 1, mRNA. |
| 6510170 | IFIT3 | 3437 | Homo sapiens interferon-induced protein with |
| | | | tetratricopeptide repeats 3 (IFIT3), mRNA. |
| 1500280 | IFIT3 | 3437 | Homo sapiens interferon-induced protein with |
| | | | tetratricopeptide repeats 3 (IFIT3), mRNA. |
| 4180544 | ROPN1L | 83853 | Homo sapiens ropporin 1-like (ROPN1L), mRNA. |
| 3290044 | TTC27 | 55622 | Homo sapiens tetratricopeptide repeat domain 27 |
| | | | (TTC27), mRNA. |
| 3170220 | LOC100133019 | 100133019 | PREDICTED: Homo sapiens similar to hCG1983765 |
| | | | (LOC100133019), mRNA. |
| 1440360 | GPR141 | 353345 | Homo sapiens G protein-coupled receptor 141 (GPR141), |
| | | | mRNA. |
| 610358 | PGS1 | 9489 | Homo sapiens phosphatidylglycerophosphate synthase 1 |
| | | | (PGS1), mRNA. |
| 2190184 | PRSS23 | 11098 | Homo sapiens protease, serine, 23 (PRSS23), mRNA. |
| 1400689 | JAM3 | 83700 | Homo sapiens junctional adhesion molecule 3 (JAM3), |
| | | | mRNA. |
| 7050458 | DBP | 1628 | Homo sapiens D site of albumin promoter (albumin D- |
| | | | box) binding protein (DBP), mRNA. |
| 2190121 | GGA2 | 23062 | Homo sapiens golgi associated, gamma adaptin ear |
| | | | containing, ARF binding protein 2 (GGA2), mRNA. |
| 2360164 | KLRC2 | 3822 | Homo sapiens killer cell lectin-like receptor subfamily C, |
| | | | member 2 (KLRC2), mRNA. |
| 4260338 | LOC647307 | 647307 | PREDICTED: Homo sapiens misc_RNA (LOC647307), |
| | | | miscRNA. |
| 7570575 | LOC642788 | 642788 | PREDICTED: Homo sapiens hypothetical protein |
| | | | LOC642788, transcript variant 2 (LOC642788), mRNA. |
| 540390 | ALPK1 | 80216 | Homo sapiens alpha-kinase 1 (ALPK1), mRNA. |
| 7650524 | BCAT1 | 586 | Homo sapiens branched chain aminotransferase 1, |
| | | | cytosolic (BCAT1), mRNA. |
| 7160343 | MYLK | 4638 | Homo sapiens myosin light chain kinase (MYLK), |
| | | | transcript variant 8, mRNA. |
| 5080601 | COBLL1 | 22837 | Homo sapiens COBL-like 1 (COBLL1), mRNA. |
| 6860639 | ATPGD1 | 57571 | Homo sapiens ATP-grasp domain containing 1 |
| | | | (ATPGD1), transcript variant 2, mRNA. |
| 2070369 | TREML1 | 340205 | Homo sapiens triggering receptor expressed on myeloid |
| | | | cells-like 1 (TREML1), mRNA. |
| 6280184 | MAN1C1 | 57134 | Homo sapiens mannosidase, alpha, class 1C, member 1 |
| | | | (MAN1C1), mRNA. |
| 4890092 | TSEN54 | 283989 | Homo sapiens tRNA splicing endonuclease 54 homolog |
| | | | (S. cerevisiae) (TSEN54), mRNA. |
| 4120053 | PFKFB3 | 5209 | Homo sapiens 6-phosphofructo-2-kinase/fructose-2,6- |
| | | | biphosphatase 3 (PFKFB3), mRNA. |
| 2940484 | GNB4 | 59345 | Homo sapiens guanine nucleotide binding protein (G |
| | | | protein), beta polypeptide 4 (GNB4), mRNA. |
| 4150270 | ANKRD22 | 118932 | Homo sapiens ankyrin repeat domain 22 (ANKRD22), |
| | | | mRNA. |
| 6420008 | PROS1 | 5627 | Homo sapiens protein S (alpha) (PROS1), mRNA. |
| 4230328 | CALD1 | 800 | Homo sapiens caldesmon 1 (CALD1), transcript variant |
| | | | 3, mRNA. |
| 50706 | CD40LG | 959 | Homo sapiens CD40 ligand (CD40LG), mRNA. |
| 4780253 | POLR3B | 55703 | Homo sapiens polymerase (RNA) III (DNA directed) |
| | | | polypeptide B (POLR3B), mRNA. |
| 3990673 | AFF1 | 4299 | Homo sapiens AF4/FMR2 family, member 1 (AFF1), |
| | | | mRNA. |
| 2140524 | HIST1H3D | 8351 | Homo sapiens histone cluster 1, H3d (HIST1H3D), |
| | | | mRNA. |
| 7610113 | SLC26A8 | 116369 | Homo sapiens solute carrier family 26, member 8 |
| | | | (SLC26A8), transcript variant 2, mRNA. |
| 5360626 | SLC26A8 | 116369 | Homo sapiens solute carrier family 26, member 8 |
| | | | (SLC26A8), transcript variant 2, mRNA. |
| 240400 | PMEPA1 | 56937 | Homo sapiens prostate transmembrane protein, androgen |
| | | | induced 1 (PMEPA1), transcript variant 2, mRNA. |
| 2070170 | UBE2L6 | 9246 | Homo sapiens ubiquitin-conjugating enzyme E2L 6 |
| | | | (UBE2L6), transcript variant 1, mRNA. |
| 7000368 | UBE2L6 | 9246 | Homo sapiens ubiquitin-conjugating enzyme E2L 6 |
| | | | (UBE2L6), transcript variant 1, mRNA. |
| 3460086 | SSH1 | 54434 | Homo sapiens slingshot homolog 1 (Drosophila) (SSH1), |
| | | | mRNA. |
| 6280092 | SLC25A23 | 79085 | Homo sapiens solute carrier family 25 (mitochondrial |
| | | | carrier; phosphate carrier), member 23 (SLC25A23), |
| | | | nuclear gene encoding mitochondrial protein, mRNA. |
| 770088 | DTX3L | 151636 | Homo sapiens deltex 3-like (Drosophila) (DTX3L), |
| | | | mRNA. |
| 1980431 | DOK3 | 79930 | Homo sapiens docking protein 3 (DOK3), mRNA. |
| 5310711 | FAM160B1 | 57700 | Homo sapiens family with sequence similarity 160, |
| | | | member B1 (FAM160B1), transcript variant 2, mRNA. |
| 4780068 | SULT1B1 | 27284 | Homo sapiens sulfotransferase family, cytosolic, 1B, |
| | | | member 1 (SULT1B1), mRNA. |
| 990044 | KIAA1147 | 57189 | PREDICTED: Homo sapiens KIAA1147 (KIAA1147), |
| | | | mRNA. |
| 5670465 | ADM | 133 | Homo sapiens adrenomedullin (ADM), mRNA. |
| 6420646 | BZRAP1 | 9256 | Homo sapiens benzodiazapine receptor (peripheral) |
| | | | associated protein 1 (BZRAP1), mRNA. |
| 620091 | LOC730234 | 730234 | PREDICTED: Homo sapiens misc_RNA (LOC730234), |
| | | | miscRNA. |
| 1820592 | HIST2H2AA3 | 8337 | Homo sapiens histone cluster 2, H2aa3 (HIST2H2AA3), |
| | | | mRNA. |
| 610451 | HIST2H2AA3 | 8337 | Homo sapiens histone cluster 2, H2aa3 (HIST2H2AA3), |
| | | | mRNA. |
| 5220142 | LIMK2 | 3985 | Homo sapiens LIM domain kinase 2 (LIMK2), transcript |
| | | | variant 2a, mRNA. |
| 1050008 | MMRN1 | 22915 | Homo sapiens multimerin 1 (MMRN1), mRNA. |
| 670528 | GYG1 | 2992 | Homo sapiens glycogenin 1 (GYG1), mRNA. |
| 6250168 | | | CR740207 NCI_CGAP_GCB1 Homo sapiens cDNA |
| | | | clone IMAGp971G2055; IMAGE: 824852 5, mRNA |
| | | | sequence |
| 3420075 | SPIN4 | 139886 | Homo sapiens spindlin family, member 4 (SPIN4), |
| | | | mRNA. |
| 4540239 | DEFA1 | 1667 | Homo sapiens defensin, alpha 1 (DEFA1), mRNA. |
| 6960397 | EPHX2 | 2053 | Homo sapiens epoxide hydrolase 2, cytoplasmic |
| | | | (EPHX2), mRNA. |
| 6480609 | RRP1B | 23076 | Homo sapiens ribosomal RNA processing 1 homolog B |
| | | | (S. cerevisiae) (RRP1B), mRNA. |
| 2570041 | WASF3 | 10810 | Homo sapiens WAS protein family, member 3 (WASF3), |
| | | | mRNA. |
| 2350747 | ABLIM3 | 22885 | Homo sapiens actin binding LIM protein family, member |
| | | | 3 (ABLIM3), mRNA. |
| 7320132 | HELB | 92797 | Homo sapiens helicase (DNA) B (HELB), mRNA. |
| 7000546 | LILRA6 | 79168 | Homo sapiens leukocyte immunoglobulin-like receptor, |
| | | | subfamily A (with TM domain), member 6 (LILRA6), |
| | | | mRNA. |
| 6060053 | LILRA6 | 79168 | Homo sapiens leukocyte immunoglobulin-like receptor, |
| | | | subfamily A (with TM domain), member 6 (LILRA6), |
| | | | mRNA. |
| 2350278 | PNOC | 5368 | Homo sapiens prepronociceptin (PNOC), mRNA. |
| 4570725 | LOC653604 | 653604 | PREDICTED: Homo sapiens similar to H3 histone, |
| | | | family 2 isoform 2 (LOC653604), mRNA. |
| 3420523 | RHBDF2 | 79651 | Homo sapiens rhomboid 5 homolog 2 (Drosophila) |
| | | | (RHBDF2), transcript variant 2, mRNA. |
| 3360500 | EGF | 1950 | Homo sapiens epidermal growth factor (beta-urogastrone) |
| | | | (EGF), mRNA. |
| 4920202 | MAP1A | 4130 | Homo sapiens microtubule-associated protein 1A |
| | | | (MAP1A), mRNA. |
| 1570468 | ZNF789 | 285989 | Homo sapiens zinc finger protein 789 (ZNF789), |
| | | | transcript variant 1, mRNA. |
| 7000184 | SLC38A9 | 153129 | Homo sapiens solute carrier family 38, member 9 |
| | | | (SLC38A9), mRNA. |
| 3520370 | MSI2 | 124540 | Homo sapiens musashi homolog 2 (Drosophila) (MSI2), |
| | | | transcript variant 1, mRNA. |
| 7000270 | PYHIN1 | 149628 | Homo sapiens pyrin and HIN domain family, member 1 |
| | | | (PYHIN1), transcript variant b2, mRNA. |
| 3870193 | TMEM180 | 79847 | Homo sapiens transmembrane protein 180 (TMEM180), |
| | | | mRNA. |
| 4200754 | ST3GAL4 | 6484 | Homo sapiens ST3 beta-galactoside alpha-2,3- |
| | | | sialyltransferase 4 (ST3GAL4), mRNA. |
| 4570612 | FASLG | 356 | Homo sapiens Fas ligand (TNF superfamily, member 6) |
| | | | (FASLG), mRNA. |
| 110196 | TMEM19 | 55266 | Homo sapiens transmembrane protein 19 (TMEM19), |
| | | | mRNA. |
| 3710086 | DHRS12 | 79758 | Homo sapiens dehydrogenase/reductase (SDR family) |
| | | | member 12 (DHRS12), transcript variant 2, mRNA. |
| 6980133 | | | HUMGS0004661 Human adult (K. Okubo) Homo sapiens |
| | | | cDNA 3, mRNA sequence |
| 5700086 | CENPK | 64105 | Homo sapiens centromere protein K (CENPK), mRNA. |
| 4040131 | LOC646909 | 646909 | PREDICTED: Homo sapiens misc_RNA (LOC646909), |
| | | | miscRNA. |
| 6590646 | FAM26F | 441168 | Homo sapiens family with sequence similarity 26, |
| | | | member F (FAM26F), mRNA. |
| 3780193 | FCRLA | 84824 | Homo sapiens Fc receptor-like A (FCRLA), mRNA. |
| 3120026 | LOC100129622 | 100129622 | PREDICTED: Homo sapiens similar to hCG1640833 |
| | | | (LOC100129622), mRNA. |
| 5690411 | MAFF | 23764 | Homo sapiens v-maf musculoaponeurotic fibrosarcoma |
| | | | oncogene homolog F (avian) (MAFF), transcript variant |
| | | | 1, mRNA. |
| 1430138 | MAFF | 23764 | Homo sapiens v-maf musculoaponeurotic fibrosarcoma |
| | | | oncogene homolog F (avian) (MAFF), transcript variant |
| | | | 1, mRNA. |
| 5290465 | CASP7 | 840 | Homo sapiens caspase 7, apoptosis-related cysteine |
| | | | peptidase (CASP7), transcript variant gamma, mRNA. |
| 2230241 | F13A1 | 2162 | Homo sapiens coagulation factor XIII, A1 polypeptide |
| | | | (F13A1), mRNA. |
| 5910594 | PTP4A3 | 11156 | Homo sapiens protein tyrosine phosphatase type IVA, |
| | | | member 3 (PTP4A3), transcript variant 2, mRNA. |
| 60524 | DENND1A | 57706 | Homo sapiens DENN/MADD domain containing 1A |
| | | | (DENND1A), transcript variant 2, mRNA. |
| 6620161 | SPIB | 6689 | Homo sapiens Spi-B transcription factor (Spi-1/PU.1 |
| | | | related) (SPIB), mRNA. |
| 2490463 | MXI1 | 4601 | Homo sapiens MAX interactor 1 (MXI1), transcript |
| | | | variant 2, mRNA. |
| 1410538 | CYP2R1 | 120227 | Homo sapiens cytochrome P450, family 2, subfamily R, |
| | | | polypeptide 1 (CYP2R1), mRNA. |
| 6580468 | RHOT1 | 55288 | Homo sapiens ras homolog gene family, member T1 |
| | | | (RHOT1), transcript variant 4, mRNA. |
| 3370400 | LOC643313 | 643313 | PREDICTED: Homo sapiens similar to hypothetical |
| | | | protein LOC284701, transcript variant 1 (LOC643313), |
| | | | mRNA. |
| 1780239 | OLIG2 | 10215 | Homo sapiens oligodendrocyte lineage transcription |
| | | | factor 2 (OLIG2), mRNA. |
| 5870259 | LOC100134440 | 100134440 | PREDICTED: Homo sapiens similar to PGBD5 protein |
| | | | (LOC100134440), mRNA. |
| 4260632 | PDZD4 | 57595 | Homo sapiens PDZ domain containing 4 (PDZD4), |
| | | | mRNA. |
| 2480328 | THOC3 | 84321 | Homo sapiens THO complex 3 (THOC3), mRNA. |
| 4900239 | CD274 | 29126 | Homo sapiens CD274 molecule (CD274), mRNA. |
| 940274 | GK | 2710 | Homo sapiens glycerol kinase (GK), transcript variant 2, |
| | | | mRNA. |
| 3940189 | JAK2 | 3717 | Homo sapiens Janus kinase 2 (a protein tyrosine kinase) |
| | | | (JAK2), mRNA. |
| 3850630 | HOMER2 | 9455 | Homo sapiens homer homolog 2 (Drosophila) |
| | | | (HOMER2), transcript variant 3, mRNA. |
| 6380672 | CA4 | 762 | Homo sapiens carbonic anhydrase IV (CA4), mRNA. |
| 6650242 | IFITM3 | 10410 | Homo sapiens interferon induced transmembrane protein |
| | | | 3 (1-8U) (IFITM3), mRNA. |
| 2070044 | CLYBL | 171425 | Homo sapiens citrate lyase beta like (CLYBL), mRNA. |
| 2470017 | LILRB3 | 11025 | Homo sapiens leukocyte immunoglobulin-like receptor, |
| | | | subfamily B (with TM and ITIM domains), member 3 |
| | | | (LILRB3), transcript variant 2, mRNA. |
| 7400376 | RABL2A | 11159 | Homo sapiens RAB, member of RAS oncogene family- |
| | | | like 2A (RABL2A), transcript variant 1, mRNA. |
| 1820750 | STAT1 | 6772 | Homo sapiens signal transducer and activator of |
| | | | transcription 1, 91 kDa (STAT1), transcript variant alpha, |
| | | | mRNA. |
| 4810187 | STAT1 | 6772 | Homo sapiens signal transducer and activator of |
| | | | transcription 1, 91 kDa (STAT1), transcript variant alpha, |
| | | | mRNA. |
| 7210672 | OR2AG1 | 144125 | Homo sapiens olfactory receptor, family 2, subfamily |
| | | | AG, member 1 (OR2AG1), mRNA. |
| 1260066 | | | Homo sapiens cDNA: FLJ21199 fis, clone COL00235 |
| 3870338 | IFI44L | 10964 | Homo sapiens interferon-induced protein 44-like |
| | | | (IFI44L), mRNA. |
| 5860452 | BNIP1 | 662 | Homo sapiens BCL2/adenovirus E1B 19 kDa interacting |
| | | | protein 1 (BNIP1), transcript variant BNIP1-c, mRNA. |
| 650619 | FAM125B | 89853 | Homo sapiens family with sequence similarity 125, |
| | | | member B (FAM125B), transcript variant 1, mRNA. |
| 6660162 | LRG1 | 116844 | Homo sapiens leucine-rich alpha-2-glycoprotein 1 |
| | | | (LRG1), mRNA. |
| 1980400 | LIMK1 | 3984 | Homo sapiens LIM domain kinase 1 (LIMK1), transcript |
| | | | variant dLIMK, mRNA. |
| 3130307 | TYMP | 1890 | Homo sapiens thymidine phosphorylase (TYMP), |
| | | | transcript variant 1, mRNA. |
| 510452 | FHIT | 2272 | Homo sapiens fragile histidine triad gene (FHIT), mRNA. |
| 7150170 | DEFA1B | 728358 | Homo sapiens defensin, alpha 1B (DEFA1B), mRNA. |
| 870477 | DEFA1B | 728358 | Homo sapiens defensin, alpha 1B (DEFA1B), mRNA. |
| 4860128 | DEFA1B | 728358 | Homo sapiens defensin, alpha 1B (DEFA1B), mRNA. |
| 6040673 | REPS2 | 9185 | Homo sapiens RALBP1 associated Eps domain |
| | | | containing 2 (REPS2), transcript variant 1, mRNA. |
| 6960022 | Sep-05 | 5413 | Homo sapiens septin 5 (SEPT5), mRNA. |
| 1850364 | ZNF550 | 162972 | Homo sapiens zinc finger protein 550 (ZNF550), mRNA. |
| 1450484 | HIST1H2BE | 8344 | Homo sapiens histone cluster 1, H2be (HIST1H2BE), |
| | | | mRNA. |
| 990349 | MCTP2 | 55784 | Homo sapiens multiple C2 domains, transmembrane 2 |
| | | | (MCTP2), mRNA. |
| 2340577 | AQP10 | 89872 | Homo sapiens aquaporin 10 (AQP10), mRNA. |
| 7400372 | WSB1 | 26118 | Homo sapiens WD repeat and SOCS box-containing 1 |
| | | | (WSB1), transcript variant 3, mRNA. |
| 6220114 | FCRL6 | 343413 | Homo sapiens Fc receptor-like 6 (FCRL6), mRNA. |
| 650546 | LOC100130604 | 100130604 | PREDICTED: Homo sapiens hypothetical protein |
| | | | LOC100130604 (LOC100130604), mRNA. |
| 3800241 | CDH6 | 1004 | Homo sapiens cadherin 6, type 2, K-cadherin (fetal |
| | | | kidney) (CDH6), mRNA. |
| 7000692 | MRPL52 | 122704 | Homo sapiens mitochondrial ribosomal protein L52 |
| | | | (MRPL52), nuclear gene encoding mitochondrial protein, |
| | | | transcript variant 6, mRNA. |
| 3870504 | ARRDC2 | 27106 | Homo sapiens arrestin domain containing 2 (ARRDC2), |
| | | | transcript variant 1, mRNA. |
| 5670523 | CIAPIN1 | 57019 | Homo sapiens cytokine induced apoptosis inhibitor 1 |
| | | | (CIAPIN1), mRNA. |
| 6100022 | HIST2H2AC | 8338 | Homo sapiens histone cluster 2, H2ac (HIST2H2AC), |
| | | | mRNA. |
| 5550474 | RAB24 | 53917 | Homo sapiens RAB24, member RAS oncogene family |
| | | | (RAB24), transcript variant 2, mRNA. |
| 6380364 | SLC7A6 | 9057 | Homo sapiens solute carrier family 7 (cationic amino acid |
| | | | transporter, y+ system), member 6 (SLC7A6), transcript |
| | | | variant 2, mRNA. |
| 5270520 | FAIM3 | 9214 | Homo sapiens Fas apoptotic inhibitory molecule 3 |
| | | | (FAIM3), mRNA. |
| 830152 | FCER2 | 2208 | Homo sapiens Fc fragment of IgE, low affinity II, |
| | | | receptor for (CD23) (FCER2), mRNA. |
| 770348 | LOC649260 | 649260 | PREDICTED: Homo sapiens similar to LIM and |
| | | | senescent cell antigen-like domains 1 (LOC649260), |
| | | | mRNA. |
| 4730164 | LOC401847 | 401847 | PREDICTED: Homo sapiens similar to hCG1793095 |
| | | | (LOC401847), mRNA. |
| 4610066 | TMEM194A | 23306 | Homo sapiens transmembrane protein 194A |
| | | | (TMEM194A), transcript variant 2, mRNA. |
| 6400333 | CTLA4 | 1493 | Homo sapiens cytotoxic T-lymphocyte-associated protein |
| | | | 4 (CTLA4), transcript variant 1, mRNA. |
| 7510537 | SCO2 | 9997 | Homo sapiens SCO cytochrome oxidase deficient |
| | | | homolog 2 (yeast) (SCO2), nuclear gene encoding |
| | | | mitochondrial protein, mRNA. |
| 2690438 | LOC100129076 | 100129076 | PREDICTED: Homo sapiens similar to FP17469 |
| | | | (LOC100129076), mRNA. |
| 940519 | GPR44 | 11251 | Homo sapiens G protein-coupled receptor 44 (GPR44), |
| | | | mRNA. |
| 1410192 | TDRD9 | 122402 | Homo sapiens tudor domain containing 9 (TDRD9), |
| | | | mRNA. |
| 6180187 | TDRD9 | 122402 | Homo sapiens tudor domain containing 9 (TDRD9), |
| | | | mRNA. |
| 380082 | LOC646144 | 646144 | PREDICTED: Homo sapiens similar to tousled-like |
| | | | kinase 2, transcript variant 2 (LOC646144), mRNA. |
| 1470470 | GPR97 | 222487 | Homo sapiens G protein-coupled receptor 97 (GPR97), |
| | | | mRNA. |
| 5260424 | GPR97 | 222487 | Homo sapiens G protein-coupled receptor 97 (GPR97), |
| | | | mRNA. |
| 4230441 | GPR97 | 222487 | Homo sapiens G protein-coupled receptor 97 (GPR97), |
| | | | mRNA. |
| 1450301 | SLAMF6 | 114836 | Homo sapiens SLAM family member 6 (SLAMF6), |
| | | | mRNA. |
| 2710152 | ODF3B | 440836 | Homo sapiens outer dense fiber of sperm tails 3B |
| | | | (ODF3B), mRNA. |
| 3460451 | SH3PXD2A | 9644 | Homo sapiens SH3 and PX domains 2A (SH3PXD2A), |
| | | | mRNA. |
| 2370743 | LOC100132858 | 100132858 | PREDICTED: Homo sapiens hypothetical protein |
| | | | LOC100132858 (LOC100132858), mRNA. |
| 6550259 | CD151 | 977 | Homo sapiens CD151 molecule (Rapp blood group) |
| | | | (CD151), transcript variant 5, mRNA. |
| 5870136 | CLIC3 | 9022 | Homo sapiens chloride intracellular channel 3 (CLIC3), |
| | | | mRNA. |
| 580452 | | | BX105747 Barstead colon HPLRB7 Homo sapiens |
| | | | cDNA clone IMAGp998G155786, mRNA sequence |
| 7400152 | P2RY10 | 27334 | Homo sapiens purinergic receptor P2Y, G-protein |
| | | | coupled, 10 (P2RY10), transcript variant 1, mRNA. |
| 6520180 | IL18RAP | 8807 | Homo sapiens interleukin 18 receptor accessory protein |
| | | | (IL18RAP), mRNA. |
| 2970747 | DEFA3 | 1668 | Homo sapiens defensin, alpha 3, neutrophil-specific |
| | | | (DEFA3), mRNA. |
| 4890598 | BRSK1 | 84446 | Homo sapiens BR serine/threonine kinase 1 (BRSK1), |
| | | | mRNA. |
| 6760593 | OSBPL10 | 114884 | Homo sapiens oxysterol binding protein-like 10 |
| | | | (OSBPL10), mRNA. |
| 1440750 | S1PR5 | 53637 | Homo sapiens sphingosine-1-phosphate receptor 5 |
| | | | (S1PR5), mRNA. |
| 1850754 | LOC644642 | 644642 | PREDICTED: Homo sapiens hypothetical protein |
| | | | LOC644642 (LOC644642), mRNA. |
| 3140487 | BMX | 660 | Homo sapiens BMX non-receptor tyrosine kinase (BMX), |
| | | | mRNA. |
| 5890288 | VEGFB | 7423 | Homo sapiens vascular endothelial growth factor B |
| | | | (VEGFB), mRNA. |
| 4150047 | LOC727962 | 727962 | PREDICTED: Homo sapiens hypothetical LOC727962 |
| | | | (LOC727962), mRNA. |
| 360343 | RHOBTB1 | 9886 | Homo sapiens Rho-related BTB domain containing 1 |
| | | | (RHOBTB1), transcript variant 2, mRNA. |
| 3990224 | TNFRSF25 | 8718 | Homo sapiens tumor necrosis factor receptor superfamily, |
| | | | member 25 (TNFRSF25), transcript variant 10, mRNA. |
| 6370014 | B3GAT1 | 27087 | Homo sapiens beta-1,3-glucuronyltransferase 1 |
| | | | (glucuronosyltransferase P) (B3GAT1), transcript variant |
| | | | 1, mRNA. |
| 6400674 | LOC730387 | 730387 | PREDICTED: Homo sapiens misc_RNA (LOC730387), |
| | | | miscRNA. |
| 4890451 | GRAMD3 | 65983 | Homo sapiens GRAM domain containing 3 (GRAMD3), |
| | | | mRNA. |
| 2570079 | STAT1 | 6772 | Homo sapiens signal transducer and activator of |
| | | | transcription 1, 91 kDa (STAT1), transcript variant beta, |
| | | | mRNA. |
| 380201 | STAP1 | 26228 | Homo sapiens signal transducing adaptor family member |
| | | | 1 (STAP1), mRNA. |
| 1570424 | WNK1 | 65125 | Homo sapiens WNK lysine deficient protein kinase 1 |
| | | | (WNK1), mRNA. |
| 4010681 | LOC374443 | 374443 | Homo sapiens CLR pseudogene (LOC374443), non- |
| | | | coding RNA. XR_001315 |
| 5360243 | ITGB5 | 3693 | Homo sapiens integrin, beta 5 (ITGB5), mRNA. |
| | | | XM_944688 XM_944693 |
| 2490411 | ITGB5 | 3693 | Homo sapiens integrin, beta 5 (ITGB5), mRNA. |
| | | | XM_944688 XM_944693 |
| 2650114 | ITGB5 | 3693 | Homo sapiens integrin, beta 5 (ITGB5), mRNA. |
| | | | XM_944688 XM_944693 |
| 4900484 | RRP15 | 51018 | Homo sapiens ribosomal RNA processing 15 homolog (S. cerevisiae) |
| | | | (RRP15), mRNA. |
| 2510561 | LOC440313 | 440313 | Homo sapiens hypothetical gene supported by AK127131 |
| | | | (LOC440313), mRNA. |
| 6350706 | ARHGAP26 | 23092 | Homo sapiens Rho GTPase activating protein 26 |
| | | | (ARHGAP26), mRNA. |
| 520086 | FCGR1A | 2209 | Homo sapiens Fc fragment of IgG, high affinity Ia, |
| | | | receptor (CD64) (FCGR1A), mRNA. |
| 2570300 | IFI44 | 10561 | Homo sapiens interferon-induced protein 44 (IFI44), |
| | | | mRNA. |
| 130161 | LCK | 3932 | Homo sapiens lymphocyte-specific protein tyrosine |
| | | | kinase (LCK), transcript variant 1, mRNA. |
| 3190537 | POLR2J3 | 548644 | Homo sapiens polymerase (RNA) II (DNA directed) |
| | | | polypeptide J3 (POLR2J3), mRNA. |
| 2710400 | DSC2 | 1824 | Homo sapiens desmocollin 2 (DSC2), transcript variant |
| | | | Dsc2b, mRNA. |
| 3060471 | ENPP4 | 22875 | Homo sapiens ectonucleotide |
| | | | pyrophosphatase/phosphodiesterase 4 (putative function) |
| | | | (ENPP4), mRNA. |
| 630619 | HPSE | 10855 | Homo sapiens heparanase (HPSE), mRNA. |
| 6620689 | MTHFD2 | 10797 | Homo sapiens methylenetetrahydrofolate dehydrogenase |
| | | | (NADP+ dependent) 2, methenyltetrahydrofolate |
| | | | cyclohydrolase (MTHFD2), nuclear gene encoding |
| | | | mitochondrial protein, transcript variant 2, mRNA. |
| 6290672 | LOC728417 | 728417 | PREDICTED: Homo sapiens hypothetical protein |
| | | | LOC728417 (LOC728417), mRNA. |
| 2570482 | LOC728417 | 728417 | PREDICTED: Homo sapiens hypothetical protein |
| | | | LOC728417 (LOC728417), mRNA. |
| 5560471 | FCAR | 2204 | Homo sapiens Fc fragment of IgA, receptor for (FCAR), |
| | | | transcript variant 10, mRNA. |
| 3420259 | MIR21 | 406991 | Homo sapiens microRNA 21 (MIR21), microRNA. |
| 3850246 | HOPX | 84525 | Homo sapiens HOP homeobox (HOPX), transcript |
| | | | variant 3, mRNA. |
| 6220372 | NSUN6 | 221078 | Homo sapiens NOL1/NOP2/Sun domain family, member |
| | | | 6 (NSUN6), mRNA. |
| 6860164 | CLEC1B | 51266 | Homo sapiens C-type lectin domain family 1, member B |
| | | | (CLEC1B), transcript variant 1, mRNA. |
| 4230228 | CDK5RAP3 | 80279 | Homo sapiens CDK5 regulatory subunit associated |
| | | | protein 3 (CDK5RAP3), transcript variant 1, mRNA. |
| 430546 | HIST1H2BG | 8339 | Homo sapiens histone cluster 1, H2bg (HIST1H2BG), |
| | | | mRNA. |
| 4250224 | LOC399491 | 399491 | PREDICTED: Homo sapiens misc_RNA (LOC399491), |
| | | | miscRNA. |
| 7100008 | C14orf80 | 283643 | Homo sapiens chromosome 14 open reading frame 80 |
| | | | (C14orf80), mRNA. |
| 3610274 | PJA1 | 64219 | Homo sapiens praja ring finger 1 (PJA1), transcript |
| | | | variant 2, mRNA. |
| 2120356 | FBXL13 | 222235 | Homo sapiens F-box and leucine-rich repeat protein 13 |
| | | | (FBXL13), mRNA. |
| 870195 | TSEN15 | 116461 | Homo sapiens tRNA splicing endonuclease 15 homolog |
| | | | (S. cerevisiae) (TSEN15), transcript variant 2, mRNA. |
| 2030170 | CARD16 | 114769 | Homo sapiens caspase recruitment domain family, |
| | | | member 16 (CARD16), transcript variant 2, mRNA. |
| 5270725 | CARD16 | 114769 | Homo sapiens caspase recruitment domain family, |
| | | | member 16 (CARD16), transcript variant 2, mRNA. |
| 870193 | SP140 | 11262 | Homo sapiens SP140 nuclear body protein (SP140), |
| | | | transcript variant 2, mRNA. |
| 6840390 | FBXL16 | 146330 | Homo sapiens F-box and leucine-rich repeat protein 16 |
| | | | (FBXL16), mRNA. |
| 6110541 | ZNF260 | 339324 | Homo sapiens zinc finger protein 260 (ZNF260), mRNA. |
| 2470364 | PRSS33 | 260429 | Homo sapiens protease, serine, 33 (PRSS33), mRNA. |
| 5570039 | LOC728744 | 728744 | PREDICTED: Homo sapiens hypothetical LOC728744 |
| | | | (LOC728744), mRNA. |
| 5720180 | FZD2 | 2535 | Homo sapiens frizzled homolog 2 (Drosophila) (FZD2), |
| | | | mRNA. |
| 4780678 | ZAP70 | 7535 | Homo sapiens zeta-chain (TCR) associated protein kinase |
| | | | 70 kDa (ZAP70), transcript variant 1, mRNA. |
| 3360372 | CCRL2 | 9034 | Homo sapiens chemokine (C-C motif) receptor-like 2 |
| | | | (CCRL2), transcript variant 2, mRNA. |
| 5390204 | SCARF1 | 8578 | Homo sapiens scavenger receptor class F, member 1 |
| | | | (SCARF1), transcript variant 3, mRNA. |
| 3990170 | IFI27 | 3429 | Homo sapiens interferon, alpha-inducible protein 27 |
| | | | (IFI27), transcript variant 2, mRNA. |
| 3120475 | MAP7 | 9053 | Homo sapiens microtubule-associated protein 7 (MAP7), |
| | | | mRNA. |
| 5310634 | FASN | 2194 | Homo sapiens fatty acid synthase (FASN), mRNA. |
| 7040181 | PFKFB2 | 5208 | Homo sapiens 6-phosphofructo-2-kinase/fructose-2,6- |
| | | | biphosphatase 2 (PFKFB2), transcript variant 2, mRNA. |
| 5870138 | VWF | 7450 | Homo sapiens von Willebrand factor (VWF), mRNA. |
| 5900543 | Sep-04 | 5414 | Homo sapiens septin 4 (SEPT4), transcript variant 3, |
| | | | mRNA. |
| 3710068 | WARS | 7453 | Homo sapiens tryptophanyl-tRNA synthetase (WARS), |
| | | | transcript variant 2, mRNA. |
| 3180528 | MMP9 | 4318 | Homo sapiens matrix metallopeptidase 9 (gelatinase B, |
| | | | 92 kDa gelatinase, 92 kDa type IV collagenase) (MMP9), |
| | | | mRNA. |
| 5560110 | DLG4 | 1742 | Homo sapiens discs, large homolog 4 (Drosophila) |
| | | | (DLG4), mRNA. |
| 6130075 | LOC391769 | 391769 | PREDICTED: Homo sapiens similar to HIStone family |
| | | | member (his-72) (LOC391769), mRNA. |
| 2650156 | TAP2 | 6891 | Homo sapiens transporter 2, ATP-binding cassette, sub- |
| | | | family B (MDR/TAP) (TAP2), transcript variant 2, |
| | | | mRNA. |
| 3120138 | F3 | 2152 | Homo sapiens coagulation factor III (thromboplastin, |
| | | | tissue factor) (F3), mRNA. |
| 2350445 | GP6 | 51206 | Homo sapiens glycoprotein VI (platelet) (GP6), mRNA. |
| 6280451 | LOC728650 | 728650 | PREDICTED: Homo sapiens misc_RNA (LOC728650), |
| | | | miscRNA. |
| 6180370 | LOC653481 | 653481 | PREDICTED: Homo sapiens similar to chromosome 6 |
| | | | open reading frame 123 (LOC653481), mRNA. |
| 2230259 | GUCY1B3 | 2983 | Homo sapiens guanylate cyclase 1, soluble, beta 3 |
| | | | (GUCY1B3), mRNA. |
| 460463 | SMARCD3 | 6604 | Homo sapiens SWI/SNF related, matrix associated, actin |
| | | | dependent regulator of chromatin, subfamily d, member 3 |
| | | | (SMARCD3), transcript variant 1, mRNA. |
| 6510131 | LOC728115 | 728115 | PREDICTED: Homo sapiens misc_RNA (LOC728115), |
| | | | miscRNA. |
| 1260133 | LOC642334 | 642334 | PREDICTED: Homo sapiens similar to hypothetical |
| | | | protein LOC284701, transcript variant 1 (LOC642334), |
| | | | mRNA. |
| 2710358 | ENOSF1 | 55556 | Homo sapiens enolase superfamily member 1 (ENOSF1), |
| | | | mRNA. |
| 6200161 | CECR6 | 27439 | Homo sapiens cat eye syndrome chromosome region, |
| | | | candidate 6 (CECR6), mRNA. |
| 110148 | BCL10 | 8915 | Homo sapiens B-cell CLL/lymphoma 10 (BCL10), |
| | | | mRNA. |
| 2370041 | LRRN3 | 54674 | Homo sapiens leucine rich repeat neuronal 3 (LRRN3), |
| | | | mRNA. |
| 4860753 | ASGR2 | 433 | Homo sapiens asialoglycoprotein receptor 2 (ASGR2), |
| | | | transcript variant 3, mRNA. |
| 3800576 | ASGR2 | 433 | Homo sapiens asialoglycoprotein receptor 2 (ASGR2), |
| | | | transcript variant 3, mRNA. |
| 2360167 | FARSA | 2193 | Homo sapiens phenylalanyl-tRNA synthetase, alpha |
| | | | subunit (FARSA), mRNA. |
| 2810241 | EXOSC5 | 56915 | Homo sapiens exosome component 5 (EXOSC5), mRNA. |
| 2600747 | IFIT2 | 3433 | Homo sapiens interferon-induced protein with |
| | | | tetratricopeptide repeats 2 (IFIT2), mRNA. |
| 2480450 | GALM | 130589 | Homo sapiens galactose mutarotase (aldose 1-epimerase) |
| | | | (GALM), mRNA. |
| 1660246 | GNB4 | 59345 | Homo sapiens guanine nucleotide binding protein (G |
| | | | protein), beta polypeptide 4 (GNB4), mRNA. |
| 5820491 | MAP7 | 9053 | Homo sapiens microtubule-associated protein 7 (MAP7), |
| | | | mRNA. |
| 1980524 | GBP4 | 115361 | Homo sapiens guanylate binding protein 4 (GBP4), |
| | | | mRNA. |
| 20075 | NDUFAF3 | 25915 | Homo sapiens NADH dehydrogenase (ubiquinone) 1 |
| | | | alpha subcomplex, assembly factor 3 (NDUFAF3), |
| | | | nuclear gene encoding mitochondrial protein, transcript |
| | | | variant 4, mRNA. |
| 3290170 | PAAF1 | 80227 | Homo sapiens proteasomal ATPase-associated factor 1 |
| | | | (PAAF1), mRNA. |
| 6520433 | ZNF789 | 285989 | Homo sapiens zinc finger protein 789 (ZNF789), |
| | | | transcript variant 2, mRNA. |
| 3460270 | ZHX3 | 23051 | Homo sapiens zinc fingers and homeoboxes 3 (ZHX3), |
| | | | mRNA. |
| 5270674 | GPR68 | 8111 | Homo sapiens G protein-coupled receptor 68 (GPR68), |
| | | | mRNA. |
| 4570441 | IFIH1 | 64135 | Homo sapiens interferon induced with helicase C domain |
| | | | 1 (IFIH1), mRNA. |
| 5810689 | PRRG4 | 79056 | Homo sapiens proline rich Gla (G-carboxyglutamic acid) |
| | | | 4 (transmembrane) (PRRG4), mRNA. |
| 4210722 | LOC641693 | 641693 | PREDICTED: Homo sapiens hypothetical protein |
| | | | LOC641693 (LOC641693), mRNA. |
| 3870170 | LOC728093 | 728093 | PREDICTED: Homo sapiens misc_RNA (LOC728093), |
| | | | partial miscRNA. |
| 1260154 | DOLPP1 | 57171 | Homo sapiens dolichyl pyrophosphate phosphatase 1 |
| | | | (DOLPP1), mRNA. |
| 3420128 | AP3M2 | 10947 | Homo sapiens adaptor-related protein complex 3, mu 2 |
| | | | subunit (AP3M2), mRNA. |
| 6900634 | CD69 | 969 | Homo sapiens CD69 molecule (CD69), mRNA. |
| 7650440 | BACH2 | 60468 | Homo sapiens BTB and CNC homology 1, basic leucine |
| | | | zipper transcription factor 2 (BACH2), mRNA. |
| 70753 | C9orf123 | 90871 | Homo sapiens chromosome 9 open reading frame 123 |
| | | | (C9orf123), mRNA. |
| 7100703 | LOC100132287 | 100132287 | PREDICTED: Homo sapiens misc_RNA |
| | | | (LOC100132287), miscRNA. |
| 2190538 | C10orf2 | 56652 | Homo sapiens chromosome 10 open reading frame 2 |
| | | | (C10orf2), mRNA. |
| 6220739 | GRAMD1B | 57476 | Homo sapiens GRAM domain containing 1B |
| | | | (GRAMD1B), mRNA. |
| 2260747 | HRK | 8739 | Homo sapiens harakiri, BCL2 interacting protein |
| | | | (contains only BH3 domain) (HRK), mRNA. |
| 5890470 | CCR6 | 1235 | Homo sapiens chemokine (C-C motif) receptor 6 (CCR6), |
| | | | transcript variant 2, mRNA. |
| 540484 | MGC16384 | 114130 | PREDICTED: Homo sapiens hypothetical protein |
| | | | MGC16384 (MGC16384), mRNA. |
| 4920110 | GADD45B | 4616 | Homo sapiens growth arrest and DNA-damage-inducible, |
| | | | beta (GADD45B), mRNA. |
| 2260131 | PLCG1 | 5335 | Homo sapiens phospholipase C, gamma 1 (PLCG1), |
| | | | transcript variant 2, mRNA. |
| 3800402 | ESAM | 90952 | Homo sapiens endothelial cell adhesion molecule |
| | | | (ESAM), mRNA. |
| 4560746 | FCAR | 2204 | Homo sapiens Fc fragment of IgA, receptor for (FCAR), |
| | | | transcript variant 3, mRNA. |
| 3440369 | CD84 | 8832 | Homo sapiens CD84 molecule (CD84), mRNA. |
| 1690044 | CA5B | 11238 | Homo sapiens carbonic anhydrase VB, mitochondrial |
| | | | (CA5B), nuclear gene encoding mitochondrial protein, |
| | | | mRNA. |
| 7510731 | WBP5 | 51186 | Homo sapiens WW domain binding protein 5 (WBP5), |
| | | | transcript variant 2, mRNA. |
| 2750674 | ASAP2 | 8853 | Homo sapiens ArfGAP with SH3 domain, ankyrin repeat |
| | | | and PH domain 2 (ASAP2), transcript variant 1, mRNA. |
| 5340646 | ASAP2 | 8853 | Homo sapiens ArfGAP with SH3 domain, ankyrin repeat |
| | | | and PH domain 2 (ASAP2), transcript variant 1, mRNA. |
| 1440341 | C1QC | 714 | Homo sapiens complement component 1, q |
| | | | subcomponent, C chain (C1QC), mRNA. |
| 1780600 | CD3E | 916 | Homo sapiens CD3e molecule, epsilon (CD3-TCR |
| | | | complex) (CD3E), mRNA. |
| 5080546 | MRPL12 | 6182 | Homo sapiens mitochondrial ribosomal protein L12 |
| | | | (MRPL12), nuclear gene encoding mitochondrial protein, |
| | | | mRNA. |
| 5670437 | GPR183 | 1880 | Homo sapiens G protein-coupled receptor 183 (GPR183), |
| | | | mRNA. |
| 4060669 | CCDC76 | 54482 | Homo sapiens coiled-coil domain containing 76 |
| | | | (CCDC76), mRNA. |
| 1190519 | MS4A1 | 931 | Homo sapiens membrane-spanning 4-domains, subfamily |
| | | | A, member 1 (MS4A1), transcript variant 3, mRNA. |
| 5720129 | NFIX | 4784 | Homo sapiens nuclear factor I/X (CCAAT-binding |
| | | | transcription factor) (NFIX), mRNA. |
| 270240 | SLC26A8 | 116369 | Homo sapiens solute carrier family 26, member 8 |
| | | | (SLC26A8), transcript variant 1, mRNA. |
| 4290037 | NOV | 4856 | Homo sapiens nephroblastoma overexpressed gene |
| | | | (NOV), mRNA. |
| 7610053 | DDX60 | 55601 | Homo sapiens DEAD (Asp-Glu-Ala-Asp) box |
| | | | polypeptide 60 (DDX60), mRNA. |
| 290152 | C16orf35 | 8131 | Homo sapiens chromosome 16 open reading frame 35 |
| | | | (C16orf35), transcript variant 1, mRNA. |
| 4860746 | LOC642684 | 642684 | PREDICTED: Homo sapiens similar to Maltase- |
| | | | glucoamylase, intestinal (LOC642684), mRNA. |
| 630397 | KIAA0319L | 79932 | Homo sapiens KIAA0319-like (KIAA0319L), transcript |
| | | | variant 1, mRNA. |
| 5960747 | TRIM22 | 10346 | Homo sapiens tripartite motif-containing 22 (TRIM22), |
| | | | mRNA. |
| 3460014 | C4orf18 | 51313 | Homo sapiens chromosome 4 open reading frame 18 |
| | | | (C4orf18), transcript variant 2, mRNA. |
| 7150433 | TCTEX1D2 | 255758 | Homo sapiens Tctex1 domain containing 2 (TCTEX1D2), |
| | | | mRNA. |
| 4610139 | SMAGP | 57228 | Homo sapiens small cell adhesion glycoprotein |
| | | | (SMAGP), transcript variant 2, mRNA. |
| 60093 | NCALD | 83988 | Homo sapiens neurocalcin delta (NCALD), mRNA. |
| 4860224 | WARS | 7453 | Homo sapiens tryptophanyl-tRNA synthetase (WARS), |
| | | | transcript variant 1, mRNA. |
| 1940162 | GBP2 | 2634 | Homo sapiens guanylate binding protein 2, interferon- |
| | | | inducible (GBP2), mRNA. |
| 1770243 | IGF2BP3 | 10643 | Homo sapiens insulin-like growth factor 2 mRNA |
| | | | binding protein 3 (IGF2BP3), mRNA. |
| 4540398 | BEND5 | 79656 | Homo sapiens BEN domain containing 5 (BEND5), |
| | | | mRNA. |
| 4220187 | DYSF | 8291 | Homo sapiens dysferlin, limb girdle muscular dystrophy |
| | | | 2B (autosomal recessive) (DYSF), mRNA. |
| 2030360 | FAM159A | 348378 | Homo sapiens family with sequence similarity 159, |
| | | | member A (FAM159A), mRNA. |
| 6180427 | GOS2 | 50486 | Homo sapiens G0/G1switch 2 (G0S2), mRNA. |
| 7330142 | TGFB1I1 | 7041 | Homo sapiens transforming growth factor beta 1 induced |
| | | | transcript 1 (TGFB1I1), transcript variant 2, mRNA. |
| 1980129 | NMNAT3 | 349565 | Homo sapiens nicotinamide nucleotide |
| | | | adenylyltransferase 3 (NMNAT3), mRNA. |
| 620750 | CALD1 | 800 | Homo sapiens caldesmon 1 (CALD1), transcript variant |
| | | | 5, mRNA. |
| 7650026 | MUC1 | 4582 | Homo sapiens mucin 1, cell surface associated (MUC1), |
| | | | transcript variant 6, mRNA. |
| 3310639 | C14orf124 | 56948 | Homo sapiens chromosome 14 open reading frame 124 |
| | | | (C14orf124), mRNA. |
| 5310377 | DSC2 | 1824 | Homo sapiens desmocollin 2 (DSC2), transcript variant |
| | | | Dsc2a, mRNA. |
| 5550397 | APOL6 | 80830 | Homo sapiens apolipoprotein L, 6 (APOL6), mRNA. |
| 3930537 | | | Human T-cell receptor (V beta 4.1-variant, J beta 2.1, C |
| | | | beta 2) mRNA |
| 7040592 | ITGAX | 3687 | PREDICTED: Homo sapiens integrin, alpha X |
| | | | (complement component 3 receptor 4 subunit) (ITGAX), |
| | | | mRNA. |
| 4540543 | SUPT3H | 8464 | Homo sapiens suppressor of Ty 3 homolog (S. cerevisiae) |
| | | | (SUPT3H), transcript variant 2, mRNA. |
| 520408 | IFIT3 | 3437 | Homo sapiens interferon-induced protein with |
| | | | tetratricopeptide repeats 3 (IFIT3), mRNA. |
| 4120367 | LPCAT2 | 54947 | Homo sapiens lysophosphatidylcholine acyltransferase 2 |
| | | | (LPCAT2), mRNA. |
| 4070600 | ZNF654 | 55279 | Homo sapiens zinc finger protein 654 (ZNF654), mRNA. |
| 430019 | LOC391045 | 391045 | PREDICTED: Homo sapiens similar to Solute carrier |
| | | | family 2, facilitated glucose transporter member 3 |
| | | | (Glucose transporter type 3, brain) (LOC391045), |
| | | | mRNA. |
| 4850600 | ZNF17 | 7565 | Homo sapiens zinc finger protein 17 (ZNF17), mRNA. |
| 1170500 | LOC402112 | 402112 | PREDICTED: Homo sapiens misc_RNA (LOC402112), |
| | | | miscRNA. |
| 1230671 | LOC100134822 | 100134822 | PREDICTED: Homo sapiens misc_RNA |
| | | | (LOC100134822), miscRNA. |
| 2690670 | ACTR5 | 79913 | Homo sapiens ARP5 actin-related protein 5 homolog |
| | | | (yeast) (ACTR5), mRNA. |
| 5270154 | LOC100131967 | 100131967 | PREDICTED: Homo sapiens misc_RNA |
| | | | (LOC100131967), miscRNA. |
| 1820689 | | | aa12g07.r1 Soares_NhHMPu_S1 Homo sapiens cDNA |
| | | | clone IMAGE: 813084 5, mRNA sequence |
| 2000064 | CTPS2 | 56474 | Homo sapiens CTP synthase II (CTPS2), transcript |
| | | | variant 2, mRNA. |
| 540092 | LOC100133551 | 100133551 | PREDICTED: Homo sapiens hypothetical protein |
| | | | LOC100133551 (LOC100133551), mRNA. |
| 7650707 | LOC729915 | 729915 | PREDICTED: Homo sapiens hypothetical LOC729915 |
| | | | (LOC729915), mRNA. |
| 1410221 | S100A12 | 6283 | Homo sapiens S100 calcium binding protein A12 |
| | | | (S100A12), mRNA. |
| 3780092 | TNFRSF21 | 27242 | Homo sapiens tumor necrosis factor receptor superfamily, |
| | | | member 21 (TNFRSF21), mRNA. |
| 6270553 | CXCL10 | 3627 | Homo sapiens chemokine (C—X—C motif) ligand 10 |
| | | | (CXCL10), mRNA. |
| 6860048 | UTS2 | 10911 | Homo sapiens urotensin 2 (UTS2), transcript variant 1, |
| | | | mRNA. |
| 3940685 | SIGLEC9 | 27180 | Homo sapiens sialic acid binding Ig-like lectin 9 |
| | | | (SIGLEC9), mRNA. |
| 990014 | C22orf32 | 91689 | Homo sapiens chromosome 22 open reading frame 32 |
| | | | (C22orf32), mRNA. |
| 6370414 | CLECL1 | 160365 | Homo sapiens C-type lectin-like 1 (CLECL1), mRNA. |
| 5890095 | LILRA5 | 353514 | Homo sapiens leukocyte immunoglobulin-like receptor, |
| | | | subfamily A (with TM domain), member 5 (LILRA5), |
| | | | transcript variant 1, mRNA. |
| 1400601 | C20orf100 | 84969 | Homo sapiens chromosome 20 open reading frame 100 |
| | | | (C20orf100), mRNA. |
| 4040187 | C6orf190 | 387357 | Homo sapiens chromosome 6 open reading frame 190 |
| | | | (C6orf190), mRNA. |
| 7650128 | ECGF1 | 1890 | Homo sapiens endothelial cell growth factor 1 (platelet- |
| | | | derived) (ECGF1), mRNA. |
| 2810615 | GRB10 | 2887 | Homo sapiens growth factor receptor-bound protein 10 |
| | | | (GRB10), transcript variant 4, mRNA. |
| 6180228 | FCRL3 | 115352 | Homo sapiens Fc receptor-like 3 (FCRL3), transcript |
| | | | variant 2, mRNA. |
| 7320348 | CLEC1A | 51267 | Homo sapiens C-type lectin domain family 1, member A |
| | | | (CLEC1A), mRNA. |
| 4670719 | ZFP91 | 80829 | Homo sapiens zinc finger protein 91 homolog (mouse) |
| | | | (ZFP91), mRNA. |
| 6380575 | INTU | 27152 | Homo sapiens inturned planar cell polarity effector |
| | | | homolog (Drosophila) (INTU), mRNA. |
| 6350131 | CTRL | 1506 | Homo sapiens chymotrypsin-like (CTRL), mRNA. |
| 5310451 | BCL6 | 604 | Homo sapiens B-cell CLL/lymphoma 6 (zinc finger |
| | | | protein 51) (BCL6), transcript variant 2, mRNA. |
| 3460348 | SAMD3 | 154075 | Homo sapiens sterile alpha motif domain containing 3 |
| | | | (SAMD3), transcript variant 2, mRNA. |
| 1430475 | EID2 | 163126 | Homo sapiens EP300 interacting inhibitor of |
| | | | differentiation 2 (EID2), mRNA. |
| 7200706 | LOXL3 | 84695 | Homo sapiens lysyl oxidase-like 3 (LOXL3), mRNA. |
| 4150369 | GK | 2710 | Homo sapiens glycerol kinase (GK), transcript variant 1, |
| | | | mRNA. |
| 6370053 | RTBDN | 83546 | Homo sapiens retbindin (RTBDN), transcript variant 2, |
| | | | mRNA. |
| 5340279 | LOC100133565 | 100133565 | PREDICTED: Homo sapiens similar to hCG23738 |
| | | | (LOC100133565), mRNA. |
| 2230204 | OAS2 | 4939 | Homo sapiens 2′-5′-oligoadenylate synthetase 2, |
| | | | 69/71 kDa (OAS2), transcript variant 3, mRNA. |
| 4250136 | LTB4R | 1241 | Homo sapiens leukotriene B4 receptor (LTB4R), mRNA. |
| 4260243 | MFHAS1 | 9258 | Homo sapiens malignant fibrous histiocytoma amplified |
| | | | sequence 1 (MFHAS1), mRNA. |
| 4040343 | LOC100129673 | 100129673 | PREDICTED: Homo sapiens similar to hCG2042915 |
| | | | (LOC100129673), mRNA. |
| 4200333 | HIST1H3E | 8353 | Homo sapiens histone cluster 1, H3e (HIST1H3E), |
| | | | mRNA. |
| 3170722 | IFT74 | 80173 | Homo sapiens intraflagellar transport 74 homolog |
| | | | (Chlamydomonas) (IFT74), transcript variant 2, mRNA. |
| 6450594 | CD79B | 974 | Homo sapiens CD79B antigen (immunoglobulin- |
| | | | associated beta) (CD79B), transcript variant 1, mRNA. |
| 5960176 | SCARNA22 | 677770 | Homo sapiens small Cajal body-specific RNA 22 |
| | | | (SCARNA22), guide RNA. |
| 450202 | ELF2 | 1998 | Homo sapiens E74-like factor 2 (ets domain transcription |
| | | | factor) (ELF2), transcript variant 1, mRNA. |
| 360154 | LOC390183 | 390183 | PREDICTED: Homo sapiens misc_RNA (LOC390183), |
| | | | miscRNA. |
| 1780224 | MGC42367 | 343990 | Homo sapiens similar to 2010300C02Rik protein |
| | | | (MGC42367), mRNA. |
| 2850639 | PRKCSH | 5589 | Homo sapiens protein kinase C substrate 80K-H |
| | | | (PRKCSH), transcript variant 2, mRNA. |
| 770474 | LPHN1 | 22859 | Homo sapiens latrophilin 1 (LPHN1), transcript variant 2, |
| | | | mRNA. |
| 4480615 | PEARL | 375033 | Homo sapiens platelet endothelial aggregation receptor 1 |
| | | | (PEAR1), mRNA. |
| 6290612 | TMEM107 | 84314 | Homo sapiens transmembrane protein 107 (TMEM107), |
| | | | transcript variant 1, mRNA. |
| 2260725 | MGAM | 8972 | Homo sapiens maltase-glucoamylase (alpha-glucosidase) |
| | | | (MGAM), mRNA. |
| 4670458 | Sep-04 | 5414 | Homo sapiens septin 4 (SEPT4), transcript variant 2, |
| | | | mRNA. |
| 240594 | ANXA3 | 306 | Homo sapiens annexin A3 (ANXA3), mRNA. |
| 6200184 | MEGF9 | 1955 | Homo sapiens multiple EGF-like-domains 9 (MEGF9), |
| | | | mRNA. |
| 2030551 | GPR27 | 2850 | Homo sapiens G protein-coupled receptor 27 (GPR27), |
| | | | mRNA. |
| 4210253 | PTPRJ | 5795 | Homo sapiens protein tyrosine phosphatase, receptor |
| | | | type, J (PTPRJ), mRNA. |
| 830296 | FAM91A2 | 57234 | PREDICTED: Homo sapiens family with sequence |
| | | | similarity 91, member A2, transcript variant 6 |
| | | | (FAM91A2), mRNA. |
| 5720300 | G3BP1 | 10146 | Homo sapiens GTPase activating protein (SH3 domain) |
| | | | binding protein 1 (G3BP1), transcript variant 1, mRNA. |
| 4920228 | FFAR2 | 2867 | Homo sapiens free fatty acid receptor 2 (FFAR2), |
| | | | mRNA. |
| 10136 | PML | 5371 | PREDICTED: Homo sapiens promyelocytic leukemia, |
| | | | transcript variant 12 (PML), mRNA. |
| 1240487 | LYRM4 | 57128 | Homo sapiens LYR motif containing 4 (LYRM4), |
| | | | mRNA. |
| 4780075 | CEACAM8 | 1088 | Homo sapiens carcinoembryonic antigen-related cell |
| | | | adhesion molecule 8 (CEACAM8), mRNA. |
| 240494 | TCF7 | 6932 | Homo sapiens transcription factor 7 (T-cell specific, |
| | | | HMG-box) (TCF7), transcript variant 5, mRNA. |
| 5700068 | TRPM2 | 7226 | Homo sapiens transient receptor potential cation channel, |
| | | | subfamily M, member 2 (TRPM2), transcript variant S, |
| | | | mRNA. |
| 1580324 | PLCD1 | 5333 | Homo sapiens phospholipase C, delta 1 (PLCD1), |
| | | | mRNA. |
| 1430068 | USF1 | 7391 | Homo sapiens upstream transcription factor 1 (USF1), |
| | | | transcript variant 1, mRNA. |
| 4150600 | EIF1AY | 9086 | Homo sapiens eukaryotic translation initiation factor 1A, |
| | | | Y-linked (EIF1AY), mRNA. |
| 4640609 | GNAZ | 2781 | Homo sapiens guanine nucleotide binding protein (G |
| | | | protein), alpha z polypeptide (GNAZ), mRNA. |
| 3940504 | CD79A | 973 | Homo sapiens CD79a molecule, immunoglobulin- |
| | | | associated alpha (CD79A), transcript variant 2, mRNA. |
| 360224 | C9orf84 | 158401 | Homo sapiens chromosome 9 open reading frame 84 |
| | | | (C9orf84), transcript variant 1, mRNA. |
| 4900154 | DPRXP4 | 503645 | Homo sapiens divergent-paired related homeobox |
| | | | pseudogene 4 (DPRXP4), non-coding RNA. |
| 3060435 | LOC652750 | 652750 | PREDICTED: Homo sapiens misc_RNA (LOC652750), |
| | | | partial miscRNA. |
| 3450521 | ECM1 | 1893 | Homo sapiens extracellular matrix protein 1 (ECM1), |
| | | | transcript variant 2, mRNA. |
| 1260601 | ST6GAL1 | 6480 | Homo sapiens ST6 beta-galactosamide alpha-2,6- |
| | | | sialyltranferase 1 (ST6GAL1), transcript variant 1, |
| | | | mRNA. |
| 2810400 | KLHL3 | 26249 | Homo sapiens kelch-like 3 (Drosophila) (KLHL3), |
| | | | mRNA. |
| 1450427 | RTP4 | 64108 | Homo sapiens receptor (chemosensory) transporter |
| | | | protein 4 (RTP4), mRNA. |
| 60554 | PLXNC1 | 10154 | Homo sapiens plexin C1 (PLXNC1), mRNA. |
| 6900528 | RHOT1 | 55288 | Homo sapiens ras homolog gene family, member T1 |
| | | | (RHOT1), transcript variant 1, mRNA. |
| 3610706 | BMX | 660 | Homo sapiens BMX non-receptor tyrosine kinase (BMX), |
| | | | mRNA. |
| 1240554 | TNFRSF17 | 608 | Homo sapiens tumor necrosis factor receptor superfamily, |
| | | | member 17 (TNFRSF17), mRNA. |
| 7380626 | FLJ20273 | 54502 | Homo sapiens RNA-binding protein (FLJ20273), mRNA. |
| 7510379 | SACS | 26278 | Homo sapiens spastic ataxia of Charlevoix-Saguenay |
| | | | (sacsin) (SACS), mRNA. |
| 3850441 | MAZ | 4150 | Homo sapiens MYC-associated zinc finger protein |
| | | | (purine-binding transcription factor) (MAZ), transcript |
| | | | variant 2, mRNA. |
| 5570187 | S1PR3 | 1903 | Homo sapiens sphingosine-1-phosphate receptor 3 |
| | | | (S1PR3), mRNA. |
| 630017 | ZNF783 | 155060 | Homo sapiens zinc finger family member 783 (ZNF783), |
| | | | mRNA. |
| 5310524 | ALG10B | 144245 | Homo sapiens asparagine-linked glycosylation 10, alpha- |
| | | | 1,2-glucosyltransferase homolog B (yeast) (ALG10B), |
| | | | mRNA. |
| 4180369 | ATP2B4 | 493 | Homo sapiens ATPase, Ca++ transporting, plasma |
| | | | membrane 4 (ATP2B4), transcript variant 1, mRNA. |
| 360360 | LOC100129343 | 100129343 | PREDICTED: Homo sapiens hypothetical protein |
| | | | LOC100129343 (LOC100129343), mRNA. |
| 7560593 | OSM | 5008 | Homo sapiens oncostatin M (OSM), mRNA. |
| 50327 | ZNF589 | 51385 | Homo sapiens zinc finger protein 589 (ZNF589), mRNA. |
| 6560411 | C9orf66 | 157983 | Homo sapiens chromosome 9 open reading frame 66 |
| | | | (C9orf66), mRNA. |
| 6480754 | FAM195B | 348262 | Homo sapiens family with sequence similarity 195, |
| | | | member B (FAM195B), transcript variant 1, mRNA. |
| 270653 | CLIC4 | 25932 | Homo sapiens chloride intracellular channel 4 (CLIC4), |
| | | | nuclear gene encoding mitochondrial protein, mRNA. |
| 7510528 | BAGE3 | 85318 | Homo sapiens B melanoma antigen family, member 3 |
| | | | (BAGE3), mRNA. |
| 20452 | TYMP | 1890 | Homo sapiens thymidine phosphorylase (TYMP), |
| | | | transcript variant 3, mRNA. |
| 1090706 | POM121L4P | 266697 | Homo sapiens POM121 membrane glycoprotein-like 4 |
| | | | pseudogene (rat) (POM121L4P), non-coding RNA. |
| 2230097 | TNFRSF25 | 8718 | Homo sapiens tumor necrosis factor receptor superfamily, |
| | | | member 25 (TNFRSF25), transcript variant 12, mRNA. |
| 5050634 | VENTX | 27287 | Homo sapiens VENT homeobox homolog (Xenopus |
| | | | laevis) (VENTX), mRNA. |
| 4280192 | TARP | 445347 | Homo sapiens TCR gamma alternate reading frame |
| | | | protein (TARP), nuclear gene encoding mitochondrial |
| | | | protein, transcript variant 2, mRNA. |
| 6250082 | HECTD2 | 143279 | Homo sapiens HECT domain containing 2 (HECTD2), |
| | | | transcript variant 2, mRNA. |
| 6840767 | FRMD3 | 257019 | Homo sapiens FERM domain containing 3 (FRMD3), |
| | | | mRNA. |
| 6100379 | LOC648984 | 648984 | PREDICTED: Homo sapiens similar to Baculoviral IAP |
| | | | repeat-containing protein 1 (Neuronal apoptosis |
| | | | inhibitory protein) (LOC648984), mRNA. |
| 6220671 | PLAUR | 5329 | Homo sapiens plasminogen activator, urokinase receptor |
| | | | (PLAUR), transcript variant 2, mRNA. |
| 360475 | PLAUR | 5329 | Homo sapiens plasminogen activator, urokinase receptor |
| | | | (PLAUR), transcript variant 2, mRNA. |
| 5420678 | LOC100132119 | 100132119 | PREDICTED: Homo sapiens misc_RNA |
| | | | (LOC100132119), miscRNA. |
| 2970019 | HIST1H4H | 8365 | Homo sapiens histone cluster 1, H4h (HIST1H4H), |
| | | | mRNA. |
| 6370523 | KBTBD8 | 84541 | Homo sapiens kelch repeat and BTB (POZ) domain |
| | | | containing 8 (KBTBD8), mRNA. |
| 630725 | IFNG | 3458 | Homo sapiens interferon, gamma (IFNG), mRNA. |
| 1050050 | C9orf45 | 81571 | PREDICTED: Homo sapiens chromosome 9 open |
| | | | reading frame 45 (C9orf45), misc RNA. |
| 4200546 | CD72 | 971 | Homo sapiens CD72 molecule (CD72), mRNA. |
| 3780047 | GBP6 | 163351 | Homo sapiens guanylate binding protein family, member |
| | | | 6 (GBP6), mRNA. |
| 1740435 | HSPC159 | 29094 | Homo sapiens galectin-related protein (HSPC159), |
| | | | mRNA. |
| 5910064 | TRIM5 | 85363 | Homo sapiens tripartite motif-containing 5 (TRIM5), |
| | | | transcript variant delta, mRNA. |
| 1300286 | MYLK | 4638 | Homo sapiens myosin light chain kinase (MYLK), |
| | | | transcript variant 2, mRNA. |
| 4230102 | SOCS3 | 9021 | Homo sapiens suppressor of cytokine signaling 3 |
| | | | (SOCS3), mRNA. |
| 2030403 | OLIG1 | 116448 | Homo sapiens oligodendrocyte transcription factor 1 |
| | | | (OLIG1), mRNA. |
| 1090739 | LIMS1 | 3987 | Homo sapiens LIM and senescent cell antigen-like |
| | | | domains 1 (LIMS1), mRNA. |
| 6060142 | AFF1 | 4299 | Homo sapiens AF4/FMR2 family, member 1 (AFF1), |
| | | | mRNA. |
| 3060524 | LOC153561 | 153561 | Homo sapiens hypothetical protein LOC153561 |
| | | | (LOC153561), mRNA. |
| 4290491 | PSME1 | 5720 | Homo sapiens proteasome (prosome, macropain) |
| | | | activator subunit 1 (PA28 alpha) (PSME1), transcript |
| | | | variant 2, mRNA. |
| 4210544 | SELPLG | 6404 | Homo sapiens selectin P ligand (SELPLG), mRNA. |
| 4810292 | CR2 | 1380 | Homo sapiens complement component (3d/Epstein Barr |
| | | | virus) receptor 2 (CR2), transcript variant 2, mRNA. |
| 1430360 | ABHD14A | 25864 | Homo sapiens abhydrolase domain containing 14A |
| | | | (ABHD14A), mRNA. |
| 2640671 | LOC642342 | 642342 | PREDICTED: Homo sapiens similar to Contactin- |
| | | | associated protein-like 3 precursor (Cell recognition |
| | | | molecule Caspr3) (LOC642342), mRNA. |
| 2650605 | C4orf18 | 51313 | Homo sapiens chromosome 4 open reading frame 18 |
| | | | (C4orf18), transcript variant 2, mRNA. |
| 2570438 | KIFC3 | 3801 | Homo sapiens kinesin family member C3 (KIFC3), |
| | | | mRNA. |
| 4040176 | LAMA5 | 3911 | Homo sapiens laminin, alpha 5 (LAMA5), mRNA. |
| 7400102 | ASPHD2 | 57168 | Homo sapiens aspartate beta-hydroxylase domain |
| | | | containing 2 (ASPHD2), mRNA. |
| 7160468 | DHRS9 | 10170 | Homo sapiens dehydrogenase/reductase (SDR family) |
| | | | member 9 (DHRS9), transcript variant 1, mRNA. |
| 10451 | BEND7 | 222389 | Homo sapiens BEN domain containing 7 (BEND7), |
| | | | transcript variant 2, mRNA. |
| 2120026 | LOC100134300 | 100134300 | PREDICTED: Homo sapiens misc_RNA |
| | | | (LOC100134300), miscRNA. |
| 1500674 | HIST2H3C | 126961 | Homo sapiens histone cluster 2, H3c (HIST2H3C), |
| | | | mRNA. |
| 2140242 | TNFAIP6 | 7130 | Homo sapiens tumor necrosis factor, alpha-induced |
| | | | protein 6 (TNFAIP6), mRNA. |
| 580739 | FBLN1 | 2192 | Homo sapiens fibulin 1 (FBLN1), transcript variant C, |
| | | | mRNA. |

TABLE 11
List of the 711 genes

| ProbeID | Symbol | Entrez_Gene_ID | Definition |
|---|---|---|---|
| 2710020 | | | BX093329 Soares_parathyroid_tumor_NbHPA Homo |
| | | | sapiens cDNA clone IMAGp998A124183; |
| | | | IMAGE: 1648403, mRNA sequence |
| 4390202 | CXCR3 | 2833 | Homo sapiens chemokine (C-X-C motif) receptor 3 |
| | | | (CXCR3), mRNA. |
| 7560114 | KLHDC8B | 200942 | Homo sapiens kelch domain containing 8B |
| | | | (KLHDC8B), mRNA. |
| 3830327 | MGLL | 11343 | Homo sapiens monoglyceride lipase (MGLL), transcript |
| | | | variant 1, mRNA. |
| 2190349 | KCNJ15 | 3772 | Homo sapiens potassium inwardly-rectifying channel, |
| | | | subfamily J, member 15 (KCNJ15), transcript variant 3, |
| | | | mRNA. |
| 4120243 | PSG9 | 5678 | Homo sapiens pregnancy specific beta-1-glycoprotein 9 |
| | | | (PSG9), mRNA. |
| 5260630 | SOCS3 | 9021 | Homo sapiens suppressor of cytokine signaling 3 |
| | | | (SOCS3), mRNA. |
| 5700725 | EPSTI1 | 94240 | Homo sapiens epithelial stromal interaction 1 (breast) |
| | | | (EPSTI1), transcript variant 2, mRNA. |
| 670594 | C2 | 717 | Homo sapiens complement component 2 (C2), mRNA. |
| 3450114 | FAM69B | 138311 | PREDICTED: Homo sapiens family with sequence |
| | | | similarity 69, member B (FAM69B), mRNA. |
| 5700070 | ALPL | 249 | Homo sapiens alkaline phosphatase, liver/bone/kidney |
| | | | (ALPL), transcript variant 1, mRNA. |
| 1340039 | TFPI | 7035 | Homo sapiens tissue factor pathway inhibitor |
| | | | (lipoprotein-associated coagulation inhibitor) (TFPI), |
| | | | transcript variant 1, mRNA. |
| 4850731 | TFPI | 7035 | Homo sapiens tissue factor pathway inhibitor |
| | | | (lipoprotein-associated coagulation inhibitor) (TFPI), |
| | | | transcript variant 1, mRNA. |
| 2070646 | GPR84 | 53831 | Homo sapiens G protein-coupled receptor 84 (GPR84), |
| | | | mRNA. |
| 3520398 | IL28RA | 163702 | Homo sapiens interleukin 28 receptor, alpha (interferon, |
| | | | lambda receptor) (IL28RA), transcript variant 1, mRNA. |
| 3850440 | FCER1G | 2207 | Homo sapiens Fc fragment of IgE, high affinity I, |
| | | | receptor for; gamma polypeptide (FCER1G), mRNA. |
| 130181 | ANKRD22 | 118932 | Homo sapiens ankyrin repeat domain 22 (ANKRD22), |
| | | | mRNA. |
| 780300 | KLHL14 | 57565 | Homo sapiens kelch-like 14 (Drosophila) (KLHL14), |
| | | | mRNA. |
| 620136 | LILRA5 | 353514 | Homo sapiens leukocyte immunoglobulin-like receptor, |
| | | | subfamily A (with TM domain), member 5 (LILRA5), |
| | | | transcript variant 3, mRNA. |
| 6130382 | PTER | 9317 | Homo sapiens phosphotriesterase related (PTER), |
| | | | transcript variant 2, mRNA. |
| 5860075 | CAMP | 820 | Homo sapiens cathelicidin antimicrobial peptide |
| | | | (CAMP), mRNA. |
| 5860253 | AHIl | 54806 | Homo sapiens Abelson helper integration site 1 (AHI1), |
| | | | mRNA. |
| 4290368 | PSTPIP2 | 9050 | Homo sapiens proline-serine-threonine phosphatase |
| | | | interacting protein 2 (PSTPIP2), mRNA. |
| 3930411 | CAMTA1 | 23261 | Homo sapiens calmodulin binding transcription activator |
| | | | 1 (CAMTA1), mRNA. |
| 2360348 | CMPK2 | 129607 | Homo sapiens cytidine monophosphate (UMP-CMP) |
| | | | kinase 2, mitochondrial (CMPK2), nuclear gene |
| | | | encoding mitochondrial protein, mRNA. |
| 2630195 | VAMP5 | 10791 | Homo sapiens vesicle-associated membrane protein 5 |
| | | | (myobrevin) (VAMPS), mRNA. |
| 2750603 | CYTH3 | 9265 | Homo sapiens cytohesin 3 (CYTH3), mRNA. |
| 2370228 | CCDC65 | 85478 | Homo sapiens coiled-coil domain containing 65 |
| | | | (CCDC65), mRNA. |
| 4290731 | KREMEN1 | 83999 | Homo sapiens kringle containing transmembrane protein |
| | | | 1 (KREMEN1), transcript variant 2, mRNA. |
| 510184 | LOC646434 | 646434 | PREDICTED: Homo sapiens hypothetical LOC646434 |
| | | | (LOC646434), mRNA. |
| 7050114 | ANKRD20A1 | 84210 | Homo sapiens ankyrin repeat domain 20 family, member |
| | | | A1 (ANKRD20A1), mRNA. |
| 6620209 | FCGR1B | 2210 | Homo sapiens Fc fragment of IgG, high affinity Ib, |
| | | | receptor (CD64) (FCGR1B), transcript variant 2, |
| | | | mRNA. |
| 5720609 | DYNC2LI1 | 51626 | Homo sapiens dynein, cytoplasmic 2, light intermediate |
| | | | chain 1 (DYNC2LI1), transcript variant 1, mRNA. |
| 130609 | FCGBP | 8857 | Homo sapiens Fc fragment of IgG binding protein |
| | | | (FCGBP), mRNA. |
| 2230575 | LOC642267 | 642267 | PREDICTED: Homo sapiens similar to hypothetical |
| | | | protein LOC284701, transcript variant 2 (LOC642267), |
| | | | mRNA. |
| 3990010 | | | AGENCOURT_7914287 NIH_MGC_71 Homo sapiens |
| | | | cDNA clone IMAGE: 6156595 5, mRNA sequence |
| 1510364 | GBP5 | 115362 | Homo sapiens guanylate binding protein 5 (GBP5), |
| | | | mRNA. |
| 7040537 | TAF9 | 6880 | Homo sapiens TAF9 RNA polymerase II, TATA box |
| | | | binding protein (TBP)-associated factor, 32 kDa (TAF9), |
| | | | transcript variant 3, mRNA. |
| 6370768 | ETV7 | 51513 | Homo sapiens ets variant 7 (ETV7), mRNA. |
| 360014 | CDH23 | 64072 | Homo sapiens cadherin-like 23 (CDH23), transcript |
| | | | variant 2, mRNA. |
| 7400037 | | | Homo sapiens T-cell receptor alpha chain-like protein |
| | | | mRNA, complete cds |
| 4060014 | LOC441124 | 441124 | PREDICTED: Homo sapiens hypothetical LOC441124 |
| | | | (LOC441124), mRNA. |
| 70730 | GAS6 | 2621 | Homo sapiens growth arrest-specific 6 (GAS6), mRNA. |
| 4480647 | TMEM50B | 757 | Homo sapiens transmembrane protein 50B |
| | | | (TMEM50B), mRNA. |
| 2680201 | | | DKFZp434O1935_r1 434 (synonym: htes3) Homo |
| | | | sapiens cDNA clone DKFZp434O1935 5, mRNA |
| | | | sequence |
| 1470091 | IL15 | 3600 | Homo sapiens interleukin 15 (ILI5), transcript variant 1, |
| | | | mRNA. |
| 2490315 | | | Homo sapiens cDNA: FLJ23098 fis, clone LNG07440 |
| 110639 | MAPK14 | 1432 | Homo sapiens mitogen-activated protein kinase 14 |
| | | | (MAPK14), transcript variant 2, mRNA. |
| 3870678 | HIST1H2AE | 3012 | Homo sapiens histone cluster 1, H2ae (HIST1H2AE), |
| | | | mRNA. |
| 5050041 | GPR109B | 8843 | Homo sapiens G protein-coupled receptor 109B |
| | | | (GPR109B), mRNA. |
| 1170300 | MT1G | 4495 | Homo sapiens metallothionein 1G (MT1G), mRNA. |
| 6900475 | ABHD2 | 11057 | Homo sapiens abhydrolase domain containing 2 |
| | | | (ABHD2), transcript variant 2, mRNA. |
| 6560156 | DUSP3 | 1845 | Homo sapiens dual specificity phosphatase 3 (vaccinia |
| | | | virus phosphatase VH1-related) (DUSP3), mRNA. |
| 6040577 | SLAMF8 | 56833 | Homo sapiens SLAM family member 8 (SLAMF8), |
| | | | mRNA. |
| 7570324 | ID3 | 3399 | Homo sapiens inhibitor of DNA binding 3, dominant |
| | | | negative helix-loop-helix protein (ID3), mRNA. |
| 3940356 | CR1 | 1378 | Homo sapiens complement component (3b/4b) receptor |
| | | | 1 (Knops blood group) (CR1), transcript variant S, |
| | | | mRNA. |
| 4200669 | CR1 | 1378 | Homo sapiens complement component (3b/4b) receptor |
| | | | 1 (Knops blood group) (CR1), transcript variant S, |
| | | | mRNA. |
| 7560039 | TAPBP | 6892 | Homo sapiens TAP binding protein (tapasin) (TAPBP), |
| | | | transcript variant 3, mRNA. |
| 5080468 | MBOAT2 | 129642 | PREDICTED: Homo sapiens membrane bound O- |
| | | | acyltransferase domain containing 2 (MBOAT2), |
| | | | mRNA. |
| 2030309 | SERPING1 | 710 | Homo sapiens serpin peptidase inhibitor, clade G (C1 |
| | | | inhibitor), member 1 (SERPING1), transcript variant 2, |
| | | | mRNA. |
| 6770707 | SQRDL | 58472 | Homo sapiens sulfide quinone reductase-like (yeast) |
| | | | (SQRDL), mRNA. |
| 5960731 | SOD2 | 6648 | Homo sapiens superoxide dismutase 2, mitochondrial |
| | | | (SOD2), nuclear gene encoding mitochondrial protein, |
| | | | transcript variant 1, mRNA. |
| 50348 | ALS2CR4 | 65062 | Homo sapiens amyotrophic lateral sclerosis 2 (juvenile) |
| | | | chromosome region, candidate 4 (ALS2CR4), transcript |
| | | | variant 1, mRNA. |
| 7570196 | TSPAN9 | 10867 | Homo sapiens tetraspanin 9 (TSPAN9), mRNA. |
| 6860291 | GASB | 2622 | Homo sapiens growth arrest-specific 8 (GAS8), mRNA. |
| 3170273 | FER1L3 | 26509 | Homo sapiens fer-1-like 3, myoferlin (C. elegans) |
| | | | (FER1L3), transcript variant 2, mRNA. |
| 1710333 | C10orf132 | 401647 | Homo sapiens chromosome 10 open reading frame 132 |
| | | | (C10orf132), mRNA. |
| 4730349 | PNKD | 25953 | Homo sapiens paroxysmal nonkinesiogenic dyskinesia |
| | | | (PNKD), transcript variant 2, mRNA. |
| 3850113 | | | df27f11.w1 Morton Fetal Cochlea Homo sapiens cDNA |
| | | | clone IMAGE:2484644 3, mRNA sequence |
| 7400603 | C15orf54 | 400360 | Homo sapiens chromosome 15 open reading frame 54 |
| | | | (C15orf54), mRNA. |
| 5420301 | | | AGENCOURT_14255630 NIH_MGC_180 Homo |
| | | | sapiens cDNA clone IMAGE:30386692 5, mRNA |
| | | | sequence |
| 580343 | ZNF540 | 163255 | Homo sapiens zinc finger protein 540 (ZNF540), |
| | | | mRNA. |
| 4010181 | FOLR3 | 2352 | Homo sapiens folate receptor 3 (gamma) (FOLR3), |
| | | | mRNA. |
| 6770762 | TPPP3 | 51673 | Homo sapiens tubulin polymerization-promoting protein |
| | | | family member 3 (TPPP3), mRNA. |
| 4810468 | SELP | 6403 | Homo sapiens selectin P (granule membrane protein |
| | | | 140 kDa, antigen CD62) (SELP), mRNA. |
| 610598 | CHI3L2 | 1117 | Homo sapiens chitinase 3-like 2 (CHI3L2), transcript |
| | | | variant 1, mRNA. |
| 2640731 | HTR3A | 3359 | Homo sapiens 5-hydroxytryptamine (serotonin) receptor |
| | | | 3A (HTR3A), transcript variant 2, mRNA. |
| 6250139 | F2RL3 | 9002 | Homo sapiens coagulation factor II (thrombin) receptor- |
| | | | like 3 (F2RL3), mRNA. |
| 6370553 | EPHA1 | 2041 | Homo sapiens EPH receptor A1 (EPHA1), mRNA. |
| 4120561 | LIMK2 | 3985 | Homo sapiens LIM domain kinase 2 (LIMK2), transcript |
| | | | variant 1, mRNA. |
| 7050370 | ANKRD9 | 122416 | Homo sapiens ankyrin repeat domain 9 (ANKRD9), |
| | | | mRNA. |
| 3360491 | MCTP1 | 79772 | Homo sapiens multiple C2 domains, transmembrane 1 |
| | | | (MCTP1), transcript variant L, mRNA. |
| 1010181 | SAMD8 | 142891 | Homo sapiens sterile alpha motif domain containing 8 |
| | | | (SAMD8), mRNA. |
| 3450452 | KIF1B | 23095 | Homo sapiens kinesin family member 1B (KIF1B), |
| | | | transcript variant 1, mRNA. |
| 4010139 | BANK1 | 55024 | Homo sapiens B-cell scaffold protein with ankyrin |
| | | | repeats 1 (BANK1), transcript variant 2, mRNA. |
| 3940482 | CDR2L | 30850 | Homo sapiens cerebellar degeneration-related protein 2- |
| | | | like (CDR2L), mRNA. |
| 7320546 | LAMP3 | 27074 | Homo sapiens lysosomal-associated membrane protein 3 |
| | | | (LAMP3), mRNA. |
| 4200719 | LOC730092 | 730092 | Homo sapiens RRN3 RNA polymerase I transcription |
| | | | factor homolog (S. cerevisiae) pseudogene |
| | | | (LOC730092) on chromosome 16. |
| 7100180 | FDXI | 2230 | Homo sapiens ferredoxin 1 (FDX1), nuclear gene |
| | | | encoding mitochondrial protein, mRNA. |
| 730008 | | | xv24e03.x1 Soares_NFL_T_GBC_S1 Homo sapiens |
| | | | cDNA clone IMAGE: 2814076 3, mRNA sequence |
| 2350661 | TSTA3 | 7264 | Homo sapiens tissue specific transplantation antigen |
| | | | P35B (TSTA3), mRNA. |
| 7560706 | C9orf109 | 286333 | PREDICTED: Homo sapiens chromosome 9 open |
| | | | reading frame 109 (C9orf109), mRNA. |
| 7510079 | CETP | 1071 | Homo sapiens cholesteryl ester transfer protein, plasma |
| | | | (CETP), mRNA. |
| 5290348 | SGCE | 8910 | Homo sapiens sarcoglycan, epsilon (SGCE), transcript |
| | | | variant 3, mRNA. |
| 150647 | NTNG2 | 84628 | Homo sapiens netrin G2 (NTNG2), mRNA. |
| 5560246 | TPM1 | 7168 | Homo sapiens tropomyosin 1 (alpha) (TPM1), transcript |
| | | | variant 3, mRNA. |
| 5270403 | | | Homo sapiens cDNA FLJ20012 fis, clone ADKA03438 |
| 5360156 | IFITM1 | 8519 | Homo sapiens interferon induced transmembrane protein |
| | | | 1 (9-27) (IFITM1), mRNA. |
| 3830228 | GPR109A | 338442 | Homo sapiens G protein-coupled receptor 109A |
| | | | (GPR109A), mRNA. |
| 2510220 | IEI35 | 3430 | Homo sapiens interferon-induced protein 35 (IEI35), |
| | | | mRNA. |
| 6400176 | IRF7 | 3665 | Homo sapiens interferon regulatory factor 7 (IRF7), |
| | | | transcript variant b, mRNA. |
| 7160164 | MATK | 4145 | Homo sapiens megakaryocyte-associated tyrosine kinase |
| | | | (MATK), transcript variant 1, mRNA. |
| 5310445 | KREMEN1 | 83999 | Homo sapiens kringle containing transmembrane protein |
| | | | 1 (KREMEN1), transcript variant 4, mRNA. |
| 3390301 | KREMEN1 | 83999 | Homo sapiens kringle containing transmembrane protein |
| | | | 1 (KREMEN1), transcript variant 4, mRNA. |
| 5310754 | VNN1 | 8876 | Homo sapiens vanin 1 (VNN1), mRNA. |
| 6840035 | GBP1 | 2633 | Homo sapiens guanylate binding protein 1, interferon- |
| | | | inducible, 67 kDa (GBPI), mRNA. |
| 2190148 | GBPI | 2633 | Homo sapiens guanylate binding protein 1, interferon- |
| | | | inducible, 67 kDa (GBP1), mRNA. |
| 6280201 | PTPRK | 5796 | Homo sapiens protein tyrosine phosphatase, receptor |
| | | | type, K (PTPRK), mRNA. |
| 6550164 | DEFA4 | 1669 | Homo sapiens defensin, alpha 4, corticostatin (DEFA4), |
| | | | mRNA. |
| 2510082 | GCET2 | 257144 | Homo sapiens germinal center expressed transcript 2 |
| | | | (GCET2), transcript variant 1, mRNA. |
| 1260424 | ADAM7 | 8756 | Homo sapiens ADAM metallopeptidase domain 7 |
| | | | (ADAM7), mRNA. |
| 3800398 | FBXO6 | 26270 | Homo sapiens F-box protein 6 (FBXO6), mRNA. |
| 5130017 | LOC730953 | 730953 | PREDICTED: Homo sapiens hypothetical protein |
| | | | LOC730953 (LOC730953), mRNA. |
| 5860066 | NMT2 | 9397 | Homo sapiens N-myristoyltransferase 2 (NMT2), |
| | | | mRNA. |
| 650753 | AFF3 | 3899 | Homo sapiens AF4/FMR2 family, member 3 (AFF3), |
| | | | transcript variant 2, mRNA. |
| 6220332 | SLC6A12 | 6539 | Homo sapiens solute carrier family 6 (neurotransmitter |
| | | | transporter, betaine/GABA), member 12 (SLC6A12), |
| | | | mRNA. |
| 130131 | ACSL4 | 2182 | Homo sapiens acyl-CoA synthetase long-chain family |
| | | | member 4 (ACSL4), transcript variant 2, mRNA. |
| 1690706 | HIST1H3H | 8357 | Homo sapiens histone cluster 1, H3h (HIST1H3H), |
| | | | mRNA. |
| 380437 | MGC52282 | 124221 | Homo sapiens hypothetical protein MGC52282 |
| | | | (MGC52282), mRNA. |
| 6510026 | IL23A | 51561 | Homo sapiens interleukin 23, alpha subunit p19 |
| | | | (IL23A), mRNA. |
| 3870458 | BRDG1 | 26228 | Homo sapiens BCR downstream signaling 1 (BRDG1), |
| | | | mRNA. |
| 6200577 | CREB5 | 9586 | Homo sapiens cAMP responsive element binding protein |
| | | | 5 (CREB5), transcript variant 1, mRNA. |
| 1740291 | TMSL8 | 11013 | Homo sapiens thymosin-like 8 (TMSL8), mRNA. |
| 4730059 | BATF2 | 116071 | Homo sapiens basic leucine zipper transcription factor, |
| | | | ATF-like 2 (BATF2), mRNA. |
| 6250092 | DTX3 | 196403 | Homo sapiens deltex 3 homolog (Drosophila) (DTX3), |
| | | | mRNA. |
| 3140689 | KIAA1407 | 57577 | Homo sapiens KIAA1407 (KIAA1407), mRNA. |
| 3420241 | SLC2A14 | 144195 | Homo sapiens solute carrier family 2 (facilitated glucose |
| | | | transporter), member 14 (SLC2A14), mRNA. |
| 2070114 | CLEC4D | 338339 | Homo sapiens C-type lectin domain family 4, member D |
| | | | (CLEC4D), mRNA. |
| 7510647 | CACNA1E | 777 | Homo sapiens calcium channel, voltage-dependent, R |
| | | | type, alpha lE subunit (CACNA1E), mRNA. |
| 4640768 | VPREB3 | 29802 | Homo sapiens pre-B lymphocyte gene 3 (VPREB3), |
| | | | mRNA. |
| 1510170 | NLRP2 | 55655 | Homo sapiens NLR family, pyrin domain containing 2 |
| | | | (NLRP2), mRNA. |
| 5340189 | | | Homo sapiens, clone IMAGE: 4655360, mRNA |
| 2100452 | NUP35 | 129401 | Homo sapiens nucleoporin 35 kDa (NUP35), mRNA. |
| 2710520 | ATPBD4 | 89978 | Homo sapiens ATP binding domain 4 (ATPBD4), |
| | | | mRNA. |
| 3800706 | TM4SF19 | 116211 | Homo sapiens transmembrane 4 L six family member 19 |
| | | | (TM4SF19), mRNA. |
| 6020520 | CASD1 | 64921 | Homo sapiens CAST domain containing 1 (CASD1), |
| | | | mRNA. |
| 730528 | PLAUR | 5329 | Homo sapiens plasminogen activator, urokinase receptor |
| | | | (PLAUR), transcript variant 1, mRNA. |
| 2450504 | LONRF3 | 79836 | Homo sapiens LON peptidase N-terminal domain and |
| | | | ring finger 3 (LONRF3), transcript variant 1, mRNA. |
| 2350438 | C6orf151 | 154007 | Homo sapiens chromosome 6 open reading frame 151 |
| | | | (C6orf151), mRNA. |
| 2970286 | CEACAM1 | 634 | Homo sapiens carcinoembryonic antigen-related cell |
| | | | adhesion molecule 1 (biliary glycoprotein) |
| | | | (CEACAM1), transcript variant 2, mRNA. |
| 5700753 | CEACAM1 | 634 | Homo sapiens carcinoembryonic antigen-related cell |
| | | | adhesion molecule 1 (biliary glycoprotein) |
| | | | (CEACAM1), transcript variant 2, mRNA. |
| 1980768 | SIAH2 | 6478 | Homo sapiens seven in absentia homolog 2 (Drosophila) |
| | | | (SIAH2), mRNA. |
| 1820307 | C19orf29 | 58509 | Homo sapiens chromosome 19 open reading frame 29 |
| | | | (C19orf29), transcript variant 1, mRNA. |
| 1820725 | LRRK2 | 120892 | Homo sapiens leucine-rich repeat kinase 2 (LRRK2), |
| | | | mRNA. |
| 4390398 | LCN2 | 3934 | Homo sapiens lipocalin 2 (LCN2), mRNA. |
| 3780441 | PHLDB2 | 90102 | Homo sapiens pleckstrin homology-like domain, family |
| | | | B, member 2 (PHLDB2), mRNA. |
| 6200193 | TncRNA | 283131 | PREDICTED: Homo sapiens trophoblast-derived |
| | | | noncoding RNA (TncRNA), misc RNA. |
| 3460053 | SIPA1L2 | 57568 | Homo sapiens signal-induced proliferation-associated 1 |
| | | | like 2 (SIPA1L2), mRNA. |
| 1240270 | LMTK2 | 22853 | Homo sapiens lemur tyrosine kinase 2 (LMTK2), |
| | | | mRNA. |
| 2230619 | HIST1H4E | 8367 | Homo sapiens histone cluster 1, H4e (HIST1H4E), |
| | | | mRNA. |
| 7650497 | ELA2 | 1991 | Homo sapiens elastase 2, neutrophil (ELA2), mRNA. |
| 6840246 | CTSA | 5476 | Homo sapiens cathepsin A (CTSA), transcript variant 1, |
| | | | mRNA. |
| 1580435 | TGM2 | 7052 | Homo sapiens transglutaminase 2 (C polypeptide, |
| | | | protein-glutamine-gamma-glutamyltransferase) (TGM2), |
| | | | transcript variant 1, mRNA. |
| 1260576 | C1orf198 | 84886 | Homo sapiens chromosome 1 open reading frame 198 |
| | | | (C1orf198), mRNA. |
| 1510026 | FLVCR2 | 55640 | Homo sapiens feline leukemia virus subgroup C cellular |
| | | | receptor family, member 2 (FLVCR2), mRNA. |
| 3420593 | LMNB1 | 4001 | Homo sapiens lamin B1 (LMNB1), mRNA. |
| 1940180 | PSMB9 | 5698 | Homo sapiens proteasome (prosome, macropain) |
| | | | subunit, beta type, 9 (large multifunctional peptidase 2) |
| | | | (PSMB9), transcript variant 1, mRNA. |
| 6450692 | PSMB9 | 5698 | Homo sapiens proteasome (prosome, macropain) |
| | | | subunit, beta type, 9 (large multifunctional peptidase 2) |
| | | | (PSMB9), transcript variant 1, mRNA. |
| 4780128 | ATF3 | 467 | Homo sapiens activating transcription factor 3 (ATF3), |
| | | | transcript variant 4, mRNA. |
| 5310411 | H2AFJ | 55766 | Homo sapiens H2A histone family, member J (H2AFJ), |
| | | | mRNA. |
| 2640025 | HP | 3240 | Homo sapiens haptoglobin (HP), mRNA. |
| 160019 | SORT1 | 6272 | Homo sapiens sortilin 1 (SORT1), mRNA. |
| 5050402 | HIST1H2BK | 85236 | Homo sapiens histone cluster 1, H2bk (HIST1H2BK), |
| | | | mRNA. |
| 6770768 | SYNGR1 | 9145 | Homo sapiens synaptogyrin 1 (SYNGRI), transcript |
| | | | variant 1b, mRNA. |
| 2650753 | KIAA1324L | 222223 | Homo sapiens KIAA1324-like (KIAA1324L), mRNA. |
| 6330471 | BLK | 640 | Homo sapiens B lymphoid tyrosine kinase (BLK), |
| | | | mRNA. |
| 2190019 | CD160 | 11126 | Homo sapiens CD160 molecule (CD160), mRNA. |
| 7380408 | dJ222E13.2 | 91695 | Homo sapiens similar to CGI-96 (dJ222E13.2) on |
| | | | chromosome 22. |
| 7210136 | CD19 | 930 | Homo sapiens CD19 molecule (CD19), mRNA. |
| 3440360 | ZNF438 | 220929 | Homo sapiens zinc finger protein 438 (ZNF438), |
| | | | mRNA. |
| 630195 | ZC3H12B | 340554 | Homo sapiens zinc finger CCCH-type containing 12B |
| | | | (ZC3H12B), mRNA. |
| 870669 | LRRC56 | 115399 | Homo sapiens leucine rich repeat containing 56 |
| | | | (LRRC56), mRNA. |
| 4480390 | SLC35D3 | 340146 | Homo sapiens solute carrier family 35, member D3 |
| | | | (SLC35D3), mRNA. |
| 4810167 | C6orf25 | 80739 | Homo sapiens chromosome 6 open reading frame 25 |
| | | | (C6orf25), transcript variant 4, mRNA. |
| 6480059 | ACTA2 | 59 | Homo sapiens actin, alpha 2, smooth muscle, aorta |
| | | | (ACTA2), mRNA. |
| 5340221 | STX16 | 8675 | Homo sapiens syntaxin 16 (STX16), transcript variant 1, |
| | | | mRNA. |
| 6040653 | TRIM6 | 117854 | Homo sapiens tripartite motif-containing 6 (TRIM6), |
| | | | transcript variant 2, mRNA. |
| 6100136 | CASP4 | 837 | Homo sapiens caspase 4, apoptosis-related cysteine |
| | | | peptidase (CASP4), transcript variant gamma, mRNA. |
| 2850735 | | | HSPD29402 HM3 Homo sapiens cDNA clone |
| | | | s5000004C11, mRNA sequence |
| 7380338 | UST | 10090 | Homo sapiens uronyl-2-sulfotransferase (UST), mRNA. |
| 5810086 | PHOSPHO2 | 493911 | Homo sapiens phosphatase, orphan 2 (PHOSPHO2), |
| | | | mRNA. |
| 130274 | LCK | 3932 | Homo sapiens lymphocyte-specific protein tyrosine |
| | | | kinase (LCK), transcript variant 2, mRNA. |
| 3420373 | SOD2 | 6648 | Homo sapiens superoxide dismutase 2, mitochondrial |
| | | | (SOD2), nuclear gene encoding mitochondrial protein, |
| | | | transcript variant 3, mRNA. |
| 5050086 | MPL | 4352 | Homo sapiens myeloproliferative leukemia virus |
| | | | oncogene (MPL), mRNA. |
| 10768 | LARGE | 9215 | Homo sapiens like-glycosyltransferase (LARGE), |
| | | | transcript variant 1, mRNA. |
| 70398 | LOC642178 | 642178 | PREDICTED: Homo sapiens similar to T-cell receptor |
| | | | beta chain V region 86T1 precursor (LOC642178), |
| | | | mRNA. |
| 4570131 | SIRT4 | 23409 | Homo sapiens sirtuin (silent mating type information |
| | | | regulation 2 homolog) 4 (S. cerevisiae) (SIRT4), mRNA. |
| 1010754 | CD22 | 933 | Homo sapiens CD22 molecule (CD22), mRNA. |
| 3710020 | EDAR | 10913 | Homo sapiens ectodysplasin A receptor (EDAR), |
| | | | mRNA. |
| 2370072 | PRR11 | 55771 | Homo sapiens proline rich 11 (PRR11), mRNA. |
| 6770543 | SEC14L5 | 9717 | Homo sapiens SEC14-like 5 (S. cerevisiae) (SEC14L5), |
| | | | mRNA. |
| 6650333 | LOC653867 | 653867 | PREDICTED: Homo sapiens similar to Occludin |
| | | | (LOC653867), mRNA. |
| 2690477 | LOC648710 | 648710 | PREDICTED: Homo sapiens similar to leukocyte |
| | | | immunoglobulin-like receptor, subfamily B (with TM |
| | | | and ITIM domains), member 3 (LOC648710), mRNA. |
| 5080010 | LOC401431 | 401431 | Homo sapiens hypothetical gene LOC401431 |
| | | | (LOC401431), mRNA. |
| 630255 | LOC652578 | 652578 | PREDICTED: Homo sapiens similar to Fc fragment of |
| | | | IgG, low affinity IIIa, receptor for (CD16) |
| | | | (LOC652578), mRNA. |
| 3360431 | ZAK | 51776 | Homo sapiens sterile alpha motif and leucine zipper |
| | | | containing kinase AZK (ZAK), transcript variant 1, |
| | | | mRNA. |
| 3940064 | KLRA1 | 10748 | Homo sapiens killer cell lectin-like receptor subfamily |
| | | | A, member 1 (KLRA1), mRNA. |
| 5720373 | ANO9 | 338440 | Homo sapiens anoctamin 9 (ANO9), mRNA. |
| 5390328 | KCNJ15 | 3772 | Homo sapiens potassium inwardly-rectifying channel, |
| | | | subfamily J, member 15 (KCNJ15), transcript variant 1, |
| | | | mRNA. |
| 1050215 | KCNJ15 | 3772 | Homo sapiens potassium inwardly-rectifying channel, |
| | | | subfamily J, member 15 (KCNJ15), transcript variant 1, |
| | | | mRNA. |
| 290762 | C21orf81 | 114035 | Homo sapiens chromosome 21 open reading frame 81 |
| | | | (C21orf81), mRNA. |
| 4260309 | MAST4 | 375449 | Homo sapiens microtubule associated serine/threonine |
| | | | kinase family member 4 (MAST4), transcript variant 2, |
| | | | mRNA. |
| 4780093 | OSGEPL1 | 64172 | Homo sapiens O-sialoglycoprotein endopeptidase-like 1 |
| | | | (OSGEPL1), mRNA. |
| 6020097 | SERPINA1 | 5265 | Homo sapiens serpin peptidase inhibitor, clade A (alpha- |
| | | | 1 antiproteinase, antitrypsin), member 1 (SERPINA1), |
| | | | transcript variant 2, mRNA. |
| 1510674 | ZFP37 | 7539 | Homo sapiens zinc finger protein 37 homolog (mouse) |
| | | | (ZFP37), mRNA. |
| 1240037 | DKFZP564O0823 | 25849 | Homo sapiens DKFZP564O0823 protein |
| | | | (DKFZP564O0823), mRNA. |
| 620403 | LOC400759 | 400759 | Homo sapiens similar to Interferon-induced guanylate- |
| | | | binding protein 1 (GTP-binding protein 1) (Guanine |
| | | | nucleotide-binding protein 1) (HuGBP-1) (LOC400759) |
| | | | on chromosome 1. |
| 6280470 | LOC728519 | 728519 | PREDICTED: Homo sapiens similar to Baculoviral IAP |
| | | | repeat-containing protein 1 (Neuronal apoptosis |
| | | | inhibitory protein) (LOC728519), mRNA. |
| 6520523 | IL27 | 246778 | Homo sapiens interleukin 27 (IL27), mRNA. |
| 2630577 | CAV2 | 858 | Homo sapiens caveolin 2 (CAV2), transcript variant 1, |
| | | | mRNA. |
| 6560079 | SUCNR1 | 56670 | Homo sapiens succinate receptor 1 (SUCNR1), mRNA. |
| 5570114 | GADD45G | 10912 | Homo sapiens growth arrest and DNA-damage- |
| | | | inducible, gamma (GADD45G), mRNA. |
| 1300139 | GNG7 | 2788 | Homo sapiens guanine nucleotide binding protein (G |
| | | | protein), gamma 7 (GNG7), mRNA. |
| 4890181 | RAP1GAP | 5909 | Homo sapiens RAP1 GTPase activating protein |
| | | | (RAP1GAP), mRNA. |
| 6840020 | TNFSF13B | 10673 | Homo sapiens tumor necrosis factor (ligand) |
| | | | superfamily, member 13b (TNFSF13B), mRNA. |
| 1070767 | OAS1 | 4938 | Homo sapiens 2′,5′-oligoadenylate synthetase 1, |
| | | | 40/46 kDa (OAS1), transcript variant 1, mRNA. |
| 3930121 | LOC645232 | 645232 | PREDICTED: Homo sapiens hypothetical protein |
| | | | LOC645232 (LOC645232), mRNA. |
| 5490743 | KIF1B | 23095 | Homo sapiens kinesin family member 1B (KIF1B), |
| | | | transcript variant 2, mRNA. |
| 6110634 | ERLIN1 | 10613 | Homo sapiens ER lipid raft associated 1 (ERLIN1), |
| | | | mRNA. |
| 430050 | BAMBI | 25805 | Homo sapiens BMP and activin membrane-bound |
| | | | inhibitor homolog (Xenopus laevis) (BAMBI), mRNA. |
| 2360719 | IRAK3 | 11213 | Homo sapiens interleukin-1 receptor-associated kinase 3 |
| | | | (IRAK3), mRNA. |
| 6580711 | AEBP1 | 165 | Homo sapiens AE binding protein 1 (AEBP1), mRNA. |
| 1050020 | NLRC4 | 58484 | Homo sapiens NLR family, CARD domain containing 4 |
| | | | (NLRC4), mRNA. |
| 780131 | | | BX436458 Homo sapiens THYMUS Homo sapiens |
| | | | cDNA clone CS0CAP001YL02 5-PRIME, mRNA |
| | | | sequence |
| 2350554 | DNAH5 | 1767 | Homo sapiens dynein, axonemal, heavy chain 5 |
| | | | (DNAH5), mRNA. |
| 3390121 | CASP5 | 838 | Homo sapiens caspase 5, apoptosis-related cysteine |
| | | | peptidase (CASP5), mRNA. |
| 6290747 | NRGN | 4900 | Homo sapiens neurogranin (protein kinase C substrate, |
| | | | RC3) (NRGN), mRNA. |
| 7150270 | LRRC26 | 389816 | Homo sapiens leucine rich repeat containing 26 |
| | | | (LRRC26), mRNA. |
| 4760433 | C16orf7 | 9605 | Homo sapiens chromosome 16 open reading frame 7 |
| | | | (C16orf7), mRNA. |
| 1110592 | EBF1 | 1879 | Homo sapiens early B-cell factor 1 (EBF1), mRNA. |
| 3290458 | FEZ1 | 9638 | Homo sapiens fasciculation and elongation protein zeta 1 |
| | | | (zygin I) (FEZ1), transcript variant 1, mRNA. |
| 6650196 | E2F5 | 1875 | Homo sapiens E2F transcription factor 5, p130-binding |
| | | | (E2F5), transcript variant 1, mRNA. |
| 5050193 | RLTPR | 146206 | Homo sapiens RGD motif, leucine rich repeats, |
| | | | tropomodulin domain and proline-rich containing |
| | | | (RLTPR), mRNA. |
| 3180392 | MARCO | 8685 | Homo sapiens macrophage receptor with collagenous |
| | | | structure (MARCO), mRNA. |
| 4920095 | MKL2 | 57496 | Homo sapiens MKL/myocardin-like 2 (MKL2), mRNA. |
| 5910070 | GPR174 | 84636 | Homo sapiens G protein-coupled receptor 174 |
| | | | (GPR174), mRNA. |
| 2320689 | LOC653610 | 653610 | PREDICTED: Homo sapiens similar to Histone H2A.o |
| | | | (H2A/o) (H2A.2) (H2a-615) (LOC653610), mRNA. |
| 1230201 | CTLA4 | 1493 | Homo sapiens cytotoxic T-lymphocyte-associated |
| | | | protein 4 (CTLA4), transcript variant 1, mRNA. |
| 5130767 | MAPK14 | 1432 | Homo sapiens mitogen-activated protein kinase 14 |
| | | | (MAPK14), transcript variant 1, mRNA. |
| 840685 | IL1B | 3553 | Homo sapiens interleukin 1, beta (IL1B), mRNA. |
| 2000128 | C4BPA | 722 | Homo sapiens complement component 4 binding |
| | | | protein, alpha (C4BPA), mRNA. |
| 4230678 | HIST2H2BE | 8349 | Homo sapiens histone cluster 2, H2be (HIST2H2BE), |
| | | | mRNA. |
| 5810477 | SERINC5 | 256987 | Homo sapiens serine incorporator 5 (SERINC5), mRNA. |
| 130753 | AIM2 | 9447 | Homo sapiens absent in melanoma 2 (AIM2), mRNA. |
| 1260270 | AIM2 | 9447 | Homo sapiens absent in melanoma 2 (AIM2), mRNA. |
| 6860246 | GRAMD1C | 54762 | Homo sapiens GRAM domain containing IC |
| | | | (GRAMD1C), mRNA. |
| 1980184 | P2RY10 | 27334 | Homo sapiens purinergic receptor P2Y, G-protein |
| | | | coupled, 10 (P2RY10), transcript variant 2, mRNA. |
| 290730 | HIST1H2BD | 3017 | Homo sapiens histone cluster 1, H2bd (HIST1H2BD), |
| | | | transcript variant 2, mRNA. |
| 6200669 | HIST1H2BD | 3017 | Homo sapiens histone cluster 1, H2bd (HIST1H2BD), |
| | | | transcript variant 2, mRNA. |
| 5390427 | NAIP | 4671 | Homo sapiens NLR family, apoptosis inhibitory protein |
| | | | (NAIP), transcript variant 1, mRNA. |
| 5340240 | NAIP | 4671 | Homo sapiens NLR family, apoptosis inhibitory protein |
| | | | (NAIP), transcript variant 1, mRNA. |
| 7550358 | NELL2 | 4753 | Homo sapiens NEL-like 2 (chicken) (NELL2), mRNA. |
| 4860600 | MAPK14 | 1432 | Homo sapiens mitogen-activated protein kinase 14 |
| | | | (MAPK14), transcript variant 3, mRNA. |
| 4610136 | GPATCH4 | 54865 | Homo sapiens G patch domain containing 4 |
| | | | (GPATCH4), transcript variant 2, mRNA. |
| 6380066 | COL4A3 | 1285 | Homo sapiens collagen, type IV, alpha 3 (Goodpasture |
| | | | antigen) (COL4A3), transcript variant 5, mRNA. |
| 1010360 | GCH1 | 2643 | Homo sapiens GTP cyclohydrolase 1 (GCH1), transcript |
| | | | variant 3, mRNA. |
| 4220343 | HSF5 | 124535 | Homo sapiens heat shock transcription factor family |
| | | | member 5 (HSF5), mRNA. |
| 1170653 | PDGFA | 5154 | Homo sapiens platelet-derived growth factor alpha |
| | | | polypeptide (PDGFA), transcript variant 2, mRNA. |
| 3360575 | TUBA8 | 51807 | Homo sapiens tubulin, alpha 8 (TUBA8), mRNA. |
| 2230538 | LRRN3 | 54674 | Homo sapiens leucine rich repeat neuronal 3 (LRRN3), |
| | | | transcript variant 1, mRNA. |
| 510707 | | | EST51770 Gall bladder I Homo sapiens cDNA 5 end, |
| | | | mRNA sequence |
| 4010270 | LOC440731 | 440731 | PREDICTED: Homo sapiens hypothetical LOC440731, |
| | | | transcript variant 2 (LOC440731), mRNA. |
| 60470 | STX11 | 8676 | Homo sapiens syntaxin 11 (STX11), mRNA. |
| 5910019 | C1QB | 713 | Homo sapiens complement component 1, q |
| | | | subcomponent, B chain (C1QB), mRNA. |
| 6620630 | XK | 7504 | Homo sapiens X-linked Kx blood group (McLeod |
| | | | syndrome) (XK), mRNA. |
| 5360093 | SECTM1 | 6398 | Homo sapiens secreted and transmembrane 1 |
| | | | (SECTM1), mRNA. |
| 3890400 | CXCR5 | 643 | Homo sapiens chemokine (C-X-C motif) receptor 5 |
| | | | (CXCR5), transcript variant 2, mRNA. |
| 460438 | FAS | 355 | Homo sapiens Fas (TNF receptor superfamily, member |
| | | | 6) (FAS), transcript variant 3, mRNA. |
| 2710709 | FCGR1B | 2210 | Homo sapiens Fc fragment of IgG, high affinity Ib, |
| | | | receptor (CD64) (FCGR1B), transcript variant 1, |
| | | | mRNA. |
| 290368 | C4orf32 | 132720 | Homo sapiens chromosome 4 open reading frame 32 |
| | | | (C4orf32), mRNA. |
| 4040037 | EBI2 | 1880 | Homo sapiens Epstein-Barr virus induced gene 2 |
| | | | (lymphocyte-specific G protein-coupled receptor) |
| | | | (EBI2), mRNA. |
| 4490520 | EBI2 | 1880 | Homo sapiens Epstein-Barr virus induced gene 2 |
| | | | (lymphocyte-specific G protein-coupled receptor) |
| | | | (EBI2), mRNA. |
| 2120746 | | | EST10086 human nasopharynx Homo sapiens cDNA, |
| | | | mRNA sequence |
| 5390747 | SPTB | 6710 | Homo sapiens spectrin, beta, erythrocytic (SPTB), |
| | | | transcript variant 2, mRNA. |
| 4150132 | PLXNA3 | 55558 | Homo sapiens plexin A3 (PLXNA3), mRNA. |
| 2490142 | C19orf35 | 374872 | Homo sapiens chromosome 19 open reading frame 35 |
| | | | (C19orf35), mRNA. |
| 1190167 | LOC152195 | 152195 | Homo sapiens hypothetical protein LOC152195 |
| | | | (LOC152195), mRNA. |
| 3800050 | ADCY3 | 109 | Homo sapiens adenylate cyclase 3 (ADCY3), mRNA. |
| 830440 | TLR5 | 7100 | Homo sapiens toll-like receptor 5 (TLR5), mRNA. |
| 1260482 | GZMK | 3003 | Homo sapiens granzyme K (granzyme 3; tryptase II) |
| | | | (GZMK), mRNA. |
| 3930368 | INCA | 440068 | Homo sapiens inhibitory caspase recruitment domain |
| | | | (CARD) protein (INCA), mRNA. |
| 7570474 | FLJ12334 | 400946 | PREDICTED: Homo sapiens hypothetical gene |
| | | | supported by AK022396; AK097927, transcript variant |
| | | | 4 (FLJ12334), mRNA. |
| 7160750 | IIP45 | 60672 | Homo sapiens invasion inhibitory protein 45 (IIP45), |
| | | | mRNA. |
| 5260392 | PRDM1 | 639 | Homo sapiens PR domain containing 1, with ZNF |
| | | | domain (PRDM1), transcript variant 1, mRNA. |
| 270593 | GPER | 2852 | Homo sapiens G protein-coupled estrogen receptor 1 |
| | | | (GPER), transcript variant 3, mRNA. |
| 3190053 | LOC642816 | 642816 | PREDICTED: Homo sapiens similar to hypothetical |
| | | | protein LOC284701, transcript variant 1 (LOC642816), |
| | | | mRNA. |
| 360132 | LHFPL2 | 10184 | Homo sapiens lipoma HMGIC fusion partner-like 2 |
| | | | (LHFPL2), mRNA. |
| 1050025 | CR1 | 1378 | Homo sapiens complement component (3b/4b) receptor |
| | | | 1 (Knops blood group) (CR1), transcript variant F, |
| | | | mRNA. |
| 5360347 | NQO1 | 1728 | Homo sapiens NAD(P)H dehydrogenase, quinone 1 |
| | | | (NQO1), transcript variant 1, mRNA. |
| 1990427 | KIAA1026 | 23254 | Homo sapiens kazrin (KIAA1026), transcript variant B, |
| | | | mRNA. |
| 3990435 | | | BX282075 N1H_MGC_120 Homo sapiens cDNA clone |
| | | | IMAGp998M2011561; IMAGE: 5223355, mRNA |
| | | | sequence |
| 1850546 | FAM102A | 399665 | Homo sapiens family with sequence similarity 102, |
| | | | member A (FAM102A), transcript variant 1, mRNA. |
| 4220592 | CACNA2D3 | 55799 | Homo sapiens calcium channel, voltage-dependent, |
| | | | alpha 2/delta subunit 3 (CACNA2D3), mRNA. |
| 1780376 | ZNF567 | 163081 | Homo sapiens zinc finger protein 567 (ZNF567), |
| | | | mRNA. |
| 2120537 | SPOCD1 | 90853 | Homo sapiens SPOC domain containing 1 (SPOCD1), |
| | | | mRNA. |
| 6180709 | | | BX429982 Homo sapiens FETAL LIVER Homo sapiens |
| | | | cDNA clone CS0DM002YI05 5-PRIME, mRNA |
| | | | sequence |
| 6420594 | TIFA | 92610 | Homo sapiens TRAF-interacting protein with forkhead- |
| | | | associated domain (TIFA), mRNA. |
| 3870382 | SLAIN1 | 122060 | Homo sapiens SLAIN motif family, member 1 |
| | | | (SLAIN1), transcript variant 2, mRNA. |
| 6650482 | EPHA4 | 2043 | Homo sapiens EPH receptor A4 (EPHA4), mRNA. |
| 4390070 | EIF1AY | 9086 | Homo sapiens eukaryotic translation initiation factor 1A, |
| | | | Y-linked (EIF1AY), mRNA. |
| 2810372 | NPR2 | 4882 | Homo sapiens natriuretic peptide receptor B/guanylate |
| | | | cyclase B (atrionatriuretic peptide receptor B) (NPR2), |
| | | | mRNA. |
| 1070367 | C19orf59 | 199675 | Homo sapiens chromosome 19 open reading frame 59 |
| | | | (C19orf59), mRNA. |
| 4260017 | CDK5RAP2 | 55755 | Homo sapiens CDK5 regulatory subunit associated |
| | | | protein 2 (CDK5RAP2), transcript variant 2, mRNA. |
| 3890609 | PLSCR1 | 5359 | Homo sapiens phospholipid scramblase 1 (PLSCR1), |
| | | | mRNA. |
| 3930379 | P2RY11 | 5032 | Homo sapiens purinergic receptor P2Y, G-protein |
| | | | coupled, 11 (P2RY11), mRNA. |
| 1050292 | GP9 | 2815 | Homo sapiens glycoprotein IX (platelet) (GP9), mRNA. |
| 1010139 | LHFP | 10186 | Homo sapiens lipoma HMGIC fusion partner (LHFP), |
| | | | mRNA. |
| 870202 | TNFSF10 | 8743 | Homo sapiens tumor necrosis factor (ligand) |
| | | | superfamily, member 10 (TNFSF10), mRNA. |
| 3780382 | DKFZp761E198 | 91056 | Homo sapiens DKFZp761E198 protein |
| | | | (DKFZp761E198), mRNA. |
| 5560280 | SAMD14 | 201191 | Homo sapiens sterile alpha motif domain containing 14 |
| | | | (SAMD14), mRNA. |
| 4150692 | PARP14 | 54625 | Homo sapiens poly (ADP-ribose) polymerase family, |
| | | | member 14 (PARP14), mRNA. |
| 3180209 | ANKI | 286 | Homo sapiens ankyrin 1, erythrocytic (ANK1), |
| | | | transcript variant 5, mRNA. |
| 5700735 | PARP9 | 83666 | Homo sapiens poly (ADP-ribose) polymerase family, |
| | | | member 9 (PARP9), mRNA. |
| 3140707 | PARP9 | 83666 | Homo sapiens poly (ADP-ribose) polymerase family, |
| | | | member 9 (PARP9), mRNA. |
| 1940132 | CD151 | 977 | Homo sapiens CD151 molecule (Raph blood group) |
| | | | (CD151), transcript variant 4, mRNA. |
| 780035 | SYTL2 | 54843 | Homo sapiens synaptotagmin-like 2 (SYTL2), transcript |
| | | | variant a, mRNA. |
| 4250066 | NUPL1 | 9818 | Homo sapiens nucleoporin like 1 (NUPL1), transcript |
| | | | variant 3, mRNA. |
| 6940274 | PTGDR | 5729 | Homo sapiens prostaglandin D2 receptor (DP) |
| | | | (PTGDR), mRNA. |
| 2810471 | PTGDR | 5729 | Homo sapiens prostaglandin D2 receptor (DP) |
| | | | (PTGDR), mRNA. |
| 1030431 | ACSL1 | 2180 | Homo sapiens acyl-CoA synthetase long-chain family |
| | | | member 1 (ACSL1), mRNA. |
| 3360132 | SNX20 | 124460 | Homo sapiens sorting nexin 20 (SNX20), mRNA. |
| 2120112 | TRIM9 | 114088 | Homo sapiens tripartite motif-containing 9 (TRIM9), |
| | | | transcript variant 1, mRNA. |
| 2120612 | SGPP2 | 130367 | PREDICTED: Homo sapiens sphingosine-l-phosphate |
| | | | phosphotase 2 (SGPP2), mRNA. |
| 540025 | IL4R | 3566 | Homo sapiens interleukin 4 receptor (IL4R), transcript |
| | | | variant 2, mRNA. |
| 10360 | OAS3 | 4940 | Homo sapiens 2′-5′-oligoadenylate synthetase 3, 100 kDa |
| | | | (OAS3), mRNA. |
| 990768 | OAS3 | 4940 | Homo sapiens 2′-5′-oligoadenylate synthetase 3, 100 kDa |
| | | | (OAS3), mRNA. |
| 3190521 | MS4A1 | 931 | Homo sapiens membrane-spanning 4-domains, |
| | | | subfamily A, member 1 (MS4A1), transcript variant 1, |
| | | | mRNA. |
| 60719 | KCNJ2 | 3759 | Homo sapiens potassium inwardly-rectifying channel, |
| | | | subfamily J, member 2 (KCNJ2), mRNA. |
| 3060427 | | | xa70g06.x1 NCI_CGAP_HSC2 Homo sapiens cDNA |
| | | | clone IMAGE: 2572186 3, mRNA sequence |
| 5340767 | CEACAM1 | 634 | Homo sapiens carcinoembryonic antigen-related cell |
| | | | adhesion molecule 1 (biliary glycoprotein) |
| | | | (CEACAM1), transcript variant 1, mRNA. |
| 2360110 | LOC653308 | 653308 | PREDICTED: Homo sapiens similar to N- |
| | | | acylsphingosine amidohydrolase 2, transcript variant 1 |
| | | | (LOC653308), mRNA. |
| 520647 | ZIK1 | 284307 | Homo sapiens zinc finger protein interacting with K |
| | | | protein 1 homolog (mouse) (ZIK1), mRNA. |
| 150551 | FMNL3 | 91010 | Homo sapiens formin-like 3 (FMNL3), transcript variant |
| | | | 2, mRNA. |
| 450189 | XAF1 | 54739 | Homo sapiens XIAP associated factor 1 (XAF1), |
| | | | transcript variant 2, mRNA. |
| 7610440 | XAF1 | 54739 | Homo sapiens XIAP associated factor 1 (XAF1), |
| | | | transcript variant 2, mRNA. |
| 1940373 | ZNF831 | 128611 | Homo sapiens zinc finger protein 831 (ZNF831), |
| | | | mRNA. |
| 2710646 | MSRB2 | 22921 | Homo sapiens methionine sulfoxide reductase B2 |
| | | | (MSRB2), mRNA. |
| 6380411 | 1IKZF3 | 22806 | Homo sapiens IKAROS family zinc finger 3 (Aiolos) |
| | | | (IKZF3), transcript variant 1, mRNA. |
| 3710152 | TRIM5 | 85363 | Homo sapiens tripartite motif-containing 5 (TRIM5), |
| | | | transcript variant gamma, mRNA. |
| 4040386 | APOL1 | 8542 | Homo sapiens apolipoprotein L, 1 (APOL1), transcript |
| | | | variant 2, mRNA. |
| 380050 | RHBDF2 | 79651 | Homo sapiens rhomboid 5 homolog 2 (Drosophila) |
| | | | (RHBDF2), transcript variant 1, mRNA. |
| 2570402 | KIF5C | 3800 | Homo sapiens kinesin family member 5C (KIF5C), |
| | | | mRNA. |
| 3310341 | FYB | 2533 | Homo sapiens FYN binding protein (FYB-120/130) |
| | | | (FYB), transcript variant 1, mRNA. |
| 3780445 | ABCA5 | 23461 | Homo sapiens ATP-binding cassette, sub-family A |
| | | | (ABC1), member 5 (ABCA5), transcript variant 1, |
| | | | mRNA. |
| 1240674 | DENND1A | 57706 | Homo sapiens DENN/MADD domain containing lA |
| | | | (DENND1A), transcript variant 1, mRNA. |
| 1580630 | LOC646043 | 646043 | PREDICTED: Homo sapiens similar to CG32346-PB, |
| | | | isoform B (LOC646043), mRNA. |
| 1240762 | RMND5A | 64795 | Homo sapiens required for meiotic nuclear division 5 |
| | | | homolog A (S. cerevisiae) (RMND5A), mRNA. |
| 990470 | LOC651738 | 651738 | PREDICTED: Homo sapiens hypothetical LOC651738 |
| | | | (LOC651738), mRNA. |
| 150626 | LOC641710 | 641710 | PREDICTED: Homo sapiens hypothetical protein |
| | | | LOC641710, transcript variant 1 (LOC641710), mRNA. |
| 3780228 | TRAF5 | 7188 | Homo sapiens TNF receptor-associated factor 5 |
| | | | (TRAF5), transcript variant 1, mRNA. |
| 7330392 | TAP1 | 6890 | Homo sapiens transporter 1, ATP-binding cassette, sub- |
| | | | family B (MDR/TAP) (TAP1), mRNA. |
| 7510750 | FCRL2 | 79368 | Homo sapiens Fc receptor-like 2 (FCRL2), transcript |
| | | | variant 2, mRNA. |
| 6770070 | FCRL2 | 79368 | Homo sapiens Fc receptor-like 2 (FCRL2), transcript |
| | | | variant 2, mRNA. |
| 1410730 | VSIG4 | 11326 | Homo sapiens V-set and immunoglobulin domain |
| | | | containing 4 (VSIG4), transcript variant 1, mRNA. |
| 6290356 | SERPINF1 | 5176 | Homo sapiens serpin peptidase inhibitor, clade F (alpha- |
| | | | 2 antiplasmin, pigment epithelium derived factor), |
| | | | member 1 (SERPINF1), mRNA. |
| 770681 | | | ai83g12.s1 Soares_parathyroid_tumor_NbHPA Homo |
| | | | sapiens cDNA clone IMAGE: 1387462 3, mRNA |
| | | | sequence |
| 1070368 | C17orf69 | 147081 | Homo sapiens chromosome 17 open reading frame 69 |
| | | | (C17orf69), mRNA. |
| 20475 | TBC1D19 | 55296 | Homo sapiens TBC1 domain family, member 19 |
| | | | (TBC1D1 9), mRNA. |
| 7380221 | LOC26010 | 26010 | Homo sapiens viral DNA polymerase-transactivated |
| | | | protein 6 (LOC26010), transcript variant 2, mRNA. |
| 6280301 | LOC643313 | 643313 | PREDICTED: Homo sapiens similar to hypothetical |
| | | | protein LOC284701, transcript variant 3 (LOC643313), |
| | | | mRNA. |
| 1570358 | APOL2 | 23780 | Homo sapiens apolipoprotein L, 2 (APOL2), transcript |
| | | | variant beta, mRNA. |
| 5700754 | APOL2 | 23780 | Homo sapiens apolipoprotein L, 2 (APOL2), transcript |
| | | | variant beta, mRNA. |
| 5550341 | KLRC1 | 3821 | Homo sapiens killer cell lectin-like receptor subfamily |
| | | | C, member 1 (KLRC1), transcript variant 4, mRNA. |
| 6510707 | FER1L3 | 26509 | Homo sapiens fer-1-like 3, myoferlin (C. elegans) |
| | | | (FER1L3), transcript variant 1, mRNA. |
| 4210753 | CD8B | 926 | Homo sapiens CD8b molecule (CD8B), transcript |
| | | | variant 5, mRNA. |
| 3830433 | | | BX096301 Soares retina N2b5HR Homo sapiens cDNA |
| | | | clone IMAGp998C16444, mRNA sequence |
| 7610136 | LILRB2 | 10288 | Homo sapiens leukocyte immunoglobulin-like receptor, |
| | | | subfamily B (with TM and ITIM domains), member 2 |
| | | | (LILRB2), transcript variant 2, mRNA. |
| 2750202 | | | Homo sapiens cDNA clone IMAGE: 4811759 |
| 7150370 | CEP120 | 153241 | Homo sapiens centrosomal protein 120 kDa (CEP120), |
| | | | mRNA. |
| 2030678 | HIST2H2AB | 317772 | Homo sapiens histone cluster 2, H2ab (HIST2H2AB), |
| | | | mRNA. |
| 770253 | KIAA1618 | 57714 | PREDICTED: Homo sapiens KIAA1618 (KIAA1618), |
| | | | mRNA. |
| 2000292 | SCGB3A1 | 92304 | Homo sapiens secretoglobin, family 3A, member 1 |
| | | | (SCGB3A1), mRNA. |
| 50136 | CMTM5 | 116173 | Homo sapiens CKLF-like MARVEL transmembrane |
| | | | domain containing 5 (CMTM5), transcript variant 3, |
| | | | mRNA. |
| 5050138 | NT5M | 56953 | Homo sapiens 5′,3′-nucleotidase, mitochondrial (NT5M), |
| | | | nuclear gene encoding mitochondrial protein, mRNA. |
| 4290619 | CREB5 | 9586 | Homo sapiens cAMP responsive element binding protein |
| | | | 5 (CREB5), transcript variant 4, mRNA. |
| 6590575 | SPRY2 | 10253 | Homo sapiens sprouty homolog 2 (Drosophila) |
| | | | (SPRY2), mRNA. |
| 520010 | LOC284701 | 284701 | PREDICTED: Homo sapiens hypothetical protein |
| | | | LOC284701, transcript variant 2 (LOC284701), mRNA. |
| 3360343 | RSAD2 | 91543 | Homo sapiens radical S-adenosyl methionine domain |
| | | | containing 2 (RSAD2), mRNA. |
| 7050382 | CASP1 | 834 | Homo sapiens caspase 1, apoptosis-related cysteine |
| | | | peptidase (interleukin 1, beta, convertase) (CASP1), |
| | | | transcript variant delta, mRNA. |
| 5910133 | GTF2A1 | 2957 | Homo sapiens general transcription factor IIA, 1, |
| | | | 19/37 kDa (GTF2A1), transcript variant 2, mRNA. |
| 4900482 | C20orf74 | 57186 | Homo sapiens chromosome 20 open reading frame 74 |
| | | | (C20orf74), mRNA. XM_945392 |
| 5310437 | MYL9 | 10398 | Homo sapiens myosin, light chain 9, regulatory (MYL9), |
| | | | transcript variant 1, mRNA. |
| 2480327 | MYL9 | 10398 | Homo sapiens myosin, light chain 9, regulatory (MYL9), |
| | | | transcript variant 1, mRNA. |
| 5340468 | ITGA2B | 3674 | Homo sapiens integrin, alpha 2b (platelet glycoprotein |
| | | | IIb of IIb/IIIa complex, antigen CD41B) (ITGA2B), |
| | | | mRNA. |
| 2630411 | COX11 | 1353 | Homo sapiens COX11 homolog, cytochrome c oxidase |
| | | | assembly protein (yeast) (COX11), nuclear gene |
| | | | encoding mitochondrial protein, mRNA. |
| 4760097 | LOC401720 | 401720 | Homo sapiens similar to fidgetin (LOC401720), mRNA. |
| 5270372 | RALGPS2 | 55103 | Homo sapiens Ral GEF with PH domain and SH3 |
| | | | binding motif 2 (RALGPS2), transcript variant 1, |
| | | | mRNA. |
| 1010246 | IFI6 | 2537 | Homo sapiens interferon, alpha-inducible protein 6 |
| | | | (IFI6), transcript variant 2, mRNA. |
| 1090390 | OAS1 | 4938 | Homo sapiens 2′,5′-oligoadenylate synthetase 1, |
| | | | 40/46 kDa (OAS I), transcript variant 3, mRNA. |
| 7040035 | OAS1 | 4938 | Homo sapiens 2′,5′-oligoadenylate synthetase 1, |
| | | | 40/46 kDa (OAS1), transcript variant 3, mRNA. |
| 940129 | WDR21A | 26094 | Homo sapiens WD repeat domain 21A (WDR21A), |
| | | | transcript variant 1, mRNA. |
| 2140020 | LIMK2 | 3985 | Homo sapiens LIM domain kinase 2 (LIMK2), transcript |
| | | | variant 2b, mRNA. |
| 3390048 | LIMK2 | 3985 | Homo sapiens LIM domain kinase 2 (LIMK2), transcript |
| | | | variant 2b, mRNA. |
| 5910632 | SMARCD3 | 6604 | Homo sapiens SWI/SNF related, matrix associated, actin |
| | | | dependent regulator of chromatin, subfamily d, member |
| | | | 3 (SMARCD3), transcript variant 2, mRNA. |
| 6100484 | TNNI2 | 7136 | Homo sapiens troponin I type 2 (skeletal, fast) (TNNI2), |
| | | | mRNA. |
| 3170136 | SAMD9L | 219285 | Homo sapiens sterile alpha motif domain containing 9- |
| | | | like (SAMD9L), mRNA. |
| 5260349 | NGFRAP1 | 27018 | Homo sapiens nerve growth factor receptor |
| | | | (TNFRSF16) associated protein 1 (NGFRAP1), |
| | | | transcript variant 1, mRNA. |
| 6860220 | NGFRAP1 | 27018 | Homo sapiens nerve growth factor receptor |
| | | | (TNFRSF16) associated protein 1 (NGFRAP1), |
| | | | transcript variant 1, mRNA. |
| 7050768 | OXTR | 5021 | Homo sapiens oxytocin receptor (OXTR), mRNA. |
| 7380463 | LOC153364 | 153364 | Homo sapiens similar to metallo-beta-lactamase |
| | | | superfamily protein (LOC153364), mRNA. |
| 540647 | TMEM56 | 148534 | Homo sapiens transmembrane protein 56 (TMEM56), |
| | | | mRNA. |
| 7400377 | CEACAM6 | 4680 | Homo sapiens carcinoembryonic antigen-related cell |
| | | | adhesion molecule 6 (non-specific cross reacting |
| | | | antigen) (CEACAM6), mRNA. |
| 1690440 | XIST | 7503 | Homo sapiens X (inactive)-specific transcript (non- |
| | | | protein coding) (XIST), non-coding RNA. |
| 5050537 | OPLAH | 26873 | Homo sapiens 5-oxoprolinase (ATP-hydrolysing) |
| | | | (OPLAH), mRNA. |
| 3360138 | ARL11 | 115761 | Homo sapiens ADP-ribosylation factor-like 11 (ARL11), |
| | | | mRNA. |
| 6480468 | BEX1 | 55859 | Homo sapiens brain expressed, X-linked 1 (BEX1), |
| | | | mRNA. |
| 4900747 | C11orf75 | 56935 | Homo sapiens chromosome 11 open reading frame 75 |
| | | | (C11orf75), mRNA. |
| 6400332 | CNIH4 | 29097 | Homo sapiens cornichon homolog 4 (Drosophila) |
| | | | (CNIH4), mRNA. |
| 1240358 | RAB20 | 55647 | Homo sapiens RAB20, member RAS oncogene family |
| | | | (RAB20), mRNA. |
| 1990300 | SOCS1 | 8651 | Homo sapiens suppressor of cytokine signaling 1 |
| | | | (SOCS1), mRNA. |
| 670288 | ZNF571 | 51276 | Homo sapiens zinc finger protein 571 (ZNF571), |
| | | | mRNA. |
| 7040477 | PLOD2 | 5352 | Homo sapiens procollagen-lysine, 2-oxoglutarate 5- |
| | | | dioxygenase 2 (PLOD2), transcript variant 2, mRNA. |
| 290451 | ADAM28 | 10863 | Homo sapiens ADAM metallopeptidase domain 28 |
| | | | (ADAM28), transcript variant 3, mRNA. |
| 1470685 | ITGB3 | 3690 | Homo sapiens integrin, beta 3 (platelet glycoprotein IIIa, |
| | | | antigen CD61) (ITGB3), mRNA. |
| 2940292 | BCKDHB | 594 | Homo sapiens branched chain keto acid dehydrogenase |
| | | | E1, beta polypeptide (maple syrup urine disease) |
| | | | (BCKDHB), nuclear gene encoding mitochondrial |
| | | | protein, transcript variant 1, mRNA. |
| 4220347 | LRRC1 | 55227 | Homo sapiens leucine rich repeat containing 1 (LRRC1), |
| | | | mRNA. |
| 630315 | DHRS9 | 10170 | Homo sapiens dehydrogenase/reductase (SDR family) |
| | | | member 9 (DHRS9), transcript variant 1, mRNA. |
| 1230148 | LOC649362 | 649362 | PREDICTED: Homo sapiens hypothetical protein |
| | | | LOC649362 (LOC649362), mRNA. |
| 150309 | SLC24A3 | 57419 | Homo sapiens solute carrier family 24 |
| | | | (sodium/potassium/calcium exchanger), member 3 |
| | | | (SLC24A3), mRNA. |
| 70754 | FCRL3 | 115352 | Homo sapiens Fc receptor-like 3 (FCRL3), mRNA. |
| 620167 | FCRL3 | 115352 | Homo sapiens Fc receptor-like 3 (FCRL3), mRNA. |
| 4050497 | C1orf138 | 574406 | Homo sapiens chromosome 1 open reading frame 138 |
| | | | (C1orf138), mRNA. |
| 2900427 | P2RX5 | 5026 | Homo sapiens purinergic receptor P2X, ligand-gated ion |
| | | | channel, 5 (P2RX5), transcript variant 2, mRNA. |
| 2100196 | ISG15 | 9636 | Homo sapiens ISG15 ubiquitin-like modifier (ISG15), |
| | | | mRNA. |
| 6760286 | | | RST24587 Athersys RAGE Library Homo sapiens |
| | | | cDNA, mRNA sequence |
| 1780440 | CD79A | 973 | Homo sapiens CD79a molecule, immunoglobulin- |
| | | | associated alpha (CD79A), transcript variant 1, mRNA. |
| 3130220 | TMEM158 | 25907 | Homo sapiens transmembrane protein 158 (TMEM158), |
| | | | mRNA. |
| 780332 | FAM3C | 10447 | Homo sapiens family with sequence similarity 3, |
| | | | member C (FAM3C), transcript variant 2, mRNA. |
| 3290292 | LAP3 | 51056 | Homo sapiens leucine aminopeptidase 3 (LAP3), |
| | | | mRNA. |
| 1660465 | FCRL5 | 83416 | Homo sapiens Fc receptor-like 5 (FCRL5), mRNA. |
| 3190608 | SLC6A9 | 6536 | Homo sapiens solute carrier family 6 (neurotransmitter |
| | | | transporter, glycine), member 9 (SLC6A9), transcript |
| | | | variant 3, mRNA. |
| 4900435 | TNFSF13B | 10673 | Homo sapiens tumor necrosis factor (ligand) |
| | | | superfamily, member 13b (TNFSF13B), mRNA. |
| 4850592 | P2RY14 | 9934 | Homo sapiens purinergic receptor P2Y, G-protein |
| | | | coupled, 14 (P2RY14), transcript variant 2, mRNA. |
| 1340364 | P2RY14 | 9934 | Homo sapiens purinergic receptor P2Y, G-protein |
| | | | coupled, 14 (P2RY14), transcript variant 2, mRNA. |
| 4220152 | SIRPG | 55423 | Homo sapiens signal-regulatory protein gamma |
| | | | (SIRPG), transcript variant 1, mRNA. |
| 1660504 | ZNF181 | 339318 | Homo sapiens zinc finger protein 181 (ZNF181), |
| | | | mRNA. |
| 6510170 | IFIT3 | 3437 | Homo sapiens interferon-induced protein with |
| | | | tetratricopeptide repeats 3 (IFIT3), mRNA. |
| 1500280 | IFIT3 | 3437 | Homo sapiens interferon-induced protein with |
| | | | tetratricopeptide repeats 3 (IFIT3), mRNA. |
| 1440360 | GPR141 | 353345 | Homo sapiens G protein-coupled receptor 141 |
| | | | (GPR141), mRNA. |
| 610358 | PGS1 | 9489 | Homo sapiens phosphatidylglycerophosphate synthase 1 |
| | | | (PGS1), mRNA. |
| 2190184 | PRSS23 | 11098 | Homo sapiens protease, serine, 23 (PRSS23), mRNA. |
| 1400689 | JAM3 | 83700 | Homo sapiens junctional adhesion molecule 3 (JAM3), |
| | | | mRNA. |
| 540390 | ALPK1 | 80216 | Homo sapiens alpha-kinase 1 (ALPK1), mRNA. |
| 2070369 | TREML1 | 340205 | Homo sapiens triggering receptor expressed on myeloid |
| | | | cells-like 1 (TREML1), mRNA. |
| 4120053 | PFKFB3 | 5209 | Homo sapiens 6-phosphofructo-2-kinase/fructose-2,6- |
| | | | biphosphatase 3 (PFKFB3), mRNA. |
| 4150270 | ANKRD22 | 118932 | Homo sapiens ankyrin repeat domain 22 (ANKRD22), |
| | | | mRNA. |
| 6420008 | PROS1 | 5627 | Homo sapiens protein S (alpha) (PROS1), mRNA. |
| 6650735 | | | Homo sapiens T cell receptor alpha locus, mRNA |
| | | | (cDNA clone MGC: 23964 IMAGE: 4687209), complete |
| | | | cds |
| 4230328 | CALD1 | 800 | Homo sapiens caldesmon 1 (CALD1), transcript variant |
| | | | 3, mRNA. |
| 1110195 | ZNF416 | 55659 | Homo sapiens zinc finger protein 416 (ZNF416), |
| | | | mRNA. |
| 2140524 | HIST1H3D | 8351 | Homo sapiens histone cluster 1, H3d (HIST1H3D), |
| | | | mRNA. |
| 7610113 | SLC26A8 | 116369 | Homo sapiens solute carrier family 26, member 8 |
| | | | (SLC26A8), transcript variant 2, mRNA. |
| 5360626 | SLC26A8 | 116369 | Homo sapiens solute carrier family 26, member 8 |
| | | | (SLC26A8), transcript variant 2, mRNA. |
| 240400 | PMEPA1 | 56937 | Homo sapiens prostate transmembrane protein, androgen |
| | | | induced 1 (PMEPA1), transcript variant 2, mRNA. |
| 2070170 | UBE2L6 | 9246 | Homo sapiens ubiquitin-conjugating enzyme E2L 6 |
| | | | (UBE2L6), transcript variant 1, mRNA. |
| 7000368 | UBE2L6 | 9246 | Homo sapiens ubiquitin-conjugating enzyme E2L 6 |
| | | | (UBE2L6), transcript variant 1, mRNA. |
| 770088 | DTX3L | 151636 | Homo sapiens deltex 3-like (Drosophila) (DTX3L), |
| | | | mRNA. |
| 1110450 | LOC644615 | 644615 | PREDICTED: Homo sapiens similar to Ras-related |
| | | | protein Rab-13 (LOC644615), mRNA. |
| 5310142 | METTL7B | 196410 | Homo sapiens methyltransferase like 7B (METTL7B), |
| | | | mRNA. |
| 5670465 | ADM | 133 | Homo sapiens adrenomedullin (ADM), mRNA. |
| 620484 | TNFRSF13B | 23495 | Homo sapiens tumor necrosis factor receptor |
| | | | superfamily, member 13B (TNFRSF13B), mRNA. |
| 1580048 | CAST | 831 | Homo sapiens calpastatin (CAST), transcript variant 9, |
| | | | mRNA. |
| 1820592 | HIST2H2AA3 | 8337 | Homo sapiens histone cluster 2, H2aa3 (HIST2H2AA3), |
| | | | mRNA. |
| 610451 | HIST2H2AA3 | 8337 | Homo sapiens histone cluster 2, H2aa3 (HIST2H2AA3), |
| | | | mRNA. |
| 5220142 | LIMK2 | 3985 | Homo sapiens LIM domain kinase 2 (LIMK2), transcript |
| | | | variant 2a, mRNA. |
| 1050008 | MMRN1 | 22915 | Homo sapiens multimerin 1 (MMRN1), mRNA. |
| 670528 | GYG1 | 2992 | Homo sapiens glycogenin 1 (GYG1), mRNA. |
| 4050259 | TRIM61 | 391712 | Homo sapiens tripartite motif-containing 61 (TRIM61), |
| | | | mRNA. |
| 6020129 | GP6 | 51206 | Homo sapiens glycoprotein VI (platelet) (GP6), |
| | | | transcript variant 1, mRNA. |
| 4540239 | DEFA1 | 1667 | Homo sapiens defensin, alpha 1 (DEFA1), mRNA. |
| 2140541 | LOC389816 | 389816 | Homo sapiens cytokeratin associated protein |
| | | | (LOC389816), mRNA. |
| 2350747 | ABLIM3 | 22885 | Homo sapiens actin binding LIM protein family, |
| | | | member 3 (ABLIM3), mRNA. |
| 2350278 | PNOC | 5368 | Homo sapiens prepronociceptin (PNOC), mRNA. |
| 2140138 | BCKDHB | 594 | Homo sapiens branched chain keto acid dehydrogenase |
| | | | E1, beta polypeptide (maple syrup urine disease) |
| | | | (BCKDHB), nuclear gene encoding mitochondrial |
| | | | protein, transcript variant 2, mRNA. |
| 3520349 | LOC653980 | 653980 | PREDICTED: Homo sapiens similar to BTB and CNC |
| | | | homology 1, basic leucine zipper transcription factor 2, |
| | | | transcript variant 1 (LOC653980), mRNA. |
| 5870575 | | | BX108101 NCI_CGAP_Kid11 Homo sapiens cDNA |
| | | | clone IMAGp998J155335, mRNA sequence |
| 290739 | DNASE1L3 | 1776 | Homo sapiens deoxyribonuclease I-like 3 (DNASE1L3), |
| | | | mRNA. |
| 3420523 | RHBDF2 | 79651 | Homo sapiens rhomboid 5 homolog 2 (Drosophila) |
| | | | (RHBDF2), transcript variant 2, mRNA. |
| 650750 | RHBDF2 | 79651 | Homo sapiens rhomboid 5 homolog 2 (Drosophila) |
| | | | (RHBDF2), transcript variant 2, mRNA. |
| 2320132 | MSI2 | 124540 | Homo sapiens musashi homolog 2 (Drosophila) (MSI2), |
| | | | transcript variant 1, mRNA. |
| 840369 | LBH | 81606 | Homo sapiens limb bud and heart development homolog |
| | | | (mouse) (LBH), mRNA. |
| 5340692 | STRBP | 55342 | Homo sapiens spermatid perinuclear RNA binding |
| | | | protein (STRBP), mRNA. |
| 4200754 | ST3GAL4 | 6484 | Homo sapiens ST3 beta-galactoside alpha-2,3- |
| | | | sialyltransferase 4 (ST3GAL4), mRNA. |
| 5050274 | DHRS12 | 79758 | Homo sapiens dehydrogenase/reductase (SDR family) |
| | | | member 12 (DHRS12), transcript variant 2, mRNA. |
| 6980133 | | | HUMGS0004661 Human adult (K. Okubo) Homo |
| | | | sapiens cDNA 3, mRNA sequence |
| 7320291 | IQCK | 124152 | Homo sapiens IQ motif containing K (IQCK), mRNA. |
| 6590646 | FAM26F | 441168 | Homo sapiens family with sequence similarity 26, |
| | | | member F (FAM26F), mRNA. |
| 3780193 | FCRLA | 84824 | Homo sapiens Fc receptor-like A (FCRLA), mRNA. |
| 6480040 | PODN | 127435 | Homo sapiens podocan (PODN), mRNA. |
| 3940196 | PCSK6 | 5046 | Homo sapiens proprotein convertase subtilisin/kexin |
| | | | type 6 (PCSK6), transcript variant 6, mRNA. |
| 830615 | ZDHHC19 | 131540 | Homo sapiens zinc finger, DHHC-type containing 19 |
| | | | (ZDHHC19), mRNA. |
| 1230091 | PML | 5371 | Homo sapiens promyelocytic leukemia (PML), transcript |
| | | | variant 1, mRNA. |
| 3850021 | CASP4 | 837 | Homo sapiens caspase 4, apoptosis-related cysteine |
| | | | peptidase (CASP4), transcript variant alpha, mRNA. |
| 6620161 | SPIB | 6689 | Homo sapiens Spi-B transcription factor (Spi-1/PU.1 |
| | | | related) (SPIB), mRNA. |
| 1410201 | CD59 | 966 | Homo sapiens CD59 molecule, complement regulatory |
| | | | protein (CD59), transcript variant 2, mRNA. |
| 3370400 | LOC643313 | 643313 | PREDICTED: Homo sapiens similar to hypothetical |
| | | | protein LOC284701, transcript variant 1 (LOC643313), |
| | | | mRNA. |
| 4900239 | CD274 | 29126 | Homo sapiens CD274 molecule (CD274), mRNA. |
| 940274 | GK | 2710 | Homo sapiens glycerol kinase (GK), transcript variant 2, |
| | | | mRNA. |
| 3850630 | HOMER2 | 9455 | Homo sapiens homer homolog 2 (Drosophila) |
| | | | (HOMER2), transcript variant 3, mRNA. |
| 6650242 | IFITM3 | 10410 | Homo sapiens interferon induced transmembrane protein |
| | | | 3 (1-8U) (IFITM3), mRNA. |
| 1740661 | KBTBD3 | 143879 | Homo sapiens kelch repeat and BTB (POZ) domain |
| | | | containing 3 (KBTBD3), transcript variant 2, mRNA. |
| 1820750 | STAT1 | 6772 | Homo sapiens signal transducer and activator of |
| | | | transcription 1, 91 kDa (STAT1), transcript variant alpha, |
| | | | mRNA. |
| 4810187 | STAT1 | 6772 | Homo sapiens signal transducer and activator of |
| | | | transcription 1, 91 kDa (STAT1), transcript variant alpha, |
| | | | mRNA. |
| 380259 | INDO | 3620 | Homo sapiens indoleamine-pyrrole 2,3 dioxygenase |
| | | | (INDO), mRNA. |
| 1260066 | | | Homo sapiens cDNA: FLJ21199 fis, clone COL00235 |
| 3870338 | IF144L | 10964 | Homo sapiens interferon-induced protein 44-like |
| | | | (IFI44L), mRNA. |
| 5860452 | BNIP1 | 662 | Homo sapiens BCL2/adenovirus E1B 19 kDa interacting |
| | | | protein 1 (BNIP1), transcript variant BNIP1-c, mRNA. |
| 6660162 | LRG1 | 116844 | Homo sapiens leucine-rich alpha-2-glycoprotein 1 |
| | | | (LRG1), mRNA. |
| 1980400 | LIMK1 | 3984 | Homo sapiens LIM domain kinase 1 (LIMK1), transcript |
| | | | variant dLIMK, mRNA. |
| 7150170 | LOC728358 | 728358 | Homo sapiens defensin, alpha 1 (LOC728358), mRNA. |
| 2480600 | LOC728358 | 728358 | Homo sapiens defensin, alpha 1 (LOC728358), mRNA. |
| 870477 | LOC728358 | 728358 | Homo sapiens defensin, alpha 1 (LOC728358), mRNA. |
| 4480445 | PDGFD | 80310 | Homo sapiens platelet derived growth factor D |
| | | | (PDGFD), transcript variant 1, mRNA. |
| 6960022 | September 2005 | 5413 | Homo sapiens septin 5 (SEPT5), mRNA. |
| 1500181 | BX105942 | | NCI_CGAP_GCB1 Homo sapiens cDNA |
| | | | clone IMAGp998B223204, mRNA sequence |
| 1770689 | CHTF18 | 63922 | Homo sapiens CTF18, chromosome transmission fidelity |
| | | | factor 18 homolog (S. cerevisiae) (CHTF18), mRNA. |
| 2900600 | C2orf15 | 150590 | Homo sapiens chromosome 2 open reading frame 15 |
| | | | (C2orf15), mRNA. |
| 2340577 | AQP10 | 89872 | Homo sapiens aquaporin 10 (AQP10), mRNA. |
| 6220114 | FCRL6 | 343413 | Homo sapiens Fc receptor-like 6 (FCRL6), mRNA. |
| 6100022 | HIST2H2AC | 8338 | Homo sapiens histone cluster 2, H2ac (HIST2H2AC), |
| | | | mRNA. |
| 5270520 | FAIM3 | 9214 | Homo sapiens Fas apoptotic inhibitory molecule 3 |
| | | | (FAIM3), mRNA. |
| 830152 | FCER2 | 2208 | Homo sapiens Fc fragment of IgE, low affinity II, |
| | | | receptor for (CD23) (FCER2), mRNA. |
| 6280170 | PDCD1 | 5133 | Homo sapiens programmed cell death 1 (PDCD1), |
| | | | mRNA. |
| 7510537 | SCO2 | 9997 | Homo sapiens SCO cytochrome oxidase deficient |
| | | | homolog 2 (yeast) (SCO2), nuclear gene encoding |
| | | | mitochondrial protein, mRNA. |
| 1410192 | TDRD9 | 122402 | Homo sapiens tudor domain containing 9 (TDRD9), |
| | | | mRNA. |
| 6180187 | TDRD9 | 122402 | Homo sapiens tudor domain containing 9 (TDRD9), |
| | | | mRNA. |
| 5390504 | | | AGENCOURT_10185031 NIH_MGC_107 Homo |
| | | | sapiens cDNA clone IMAGE:6569320 5, mRNA |
| | | | sequence |
| 5260424 | GPR97 | 222487 | Homo sapiens G protein-coupled receptor 97 (GPR97), |
| | | | mRNA. |
| 1470470 | GPR97 | 222487 | Homo sapiens G protein-coupled receptor 97 (GPR97), |
| | | | mRNA. |
| 4230441 | GPR97 | 222487 | Homo sapiens G protein-coupled receptor 97 (GPR97), |
| | | | mRNA. |
| 3440685 | TTN | 7273 | Homo sapiens titin (TTN), transcript variant N2-A, |
| | | | mRNA. |
| 3930128 | | | BX110640 Soares_testis_NHT Homo sapiens cDNA |
| | | | clone IMAGp998B094156, mRNA sequence |
| 620301 | KIAA0329 | 9895 | Homo sapiens KIAA0329 (KIAA0329), mRNA. |
| 6520180 | IL18RAP | 8807 | Homo sapiens interleukin 18 receptor accessory protein |
| | | | (IL18RAP), mRNA. |
| 2970747 | DEFA3 | 1668 | Homo sapiens defensin, alpha 3, neutrophil-specific |
| | | | (DEFA3), mRNA. |
| 1580400 | TTC26 | 79989 | Homo sapiens tetratricopeptide repeat domain 26 |
| | | | (TTC26), mRNA. |
| 6760593 | OSBPL10 | 114884 | Homo sapiens oxysterol binding protein-like 10 |
| | | | (OSBPL10), mRNA. |
| 3140487 | BMX | 660 | Homo sapiens BMX non-receptor tyrosine kinase |
| | | | (BMX), mRNA. |
| 360113 | | | 17000600169456 GRN_PRENEU Homo sapiens cDNA |
| | | | 5, mRNA sequence |
| 5720482 | HERC5 | 51191 | Homo sapiens hect domain and RLD 5 (HERC5), |
| | | | mRNA. |
| 2570079 | STAT1 | 6772 | Homo sapiens signal transducer and activator of |
| | | | transcription 1, 91 kDa (STAT1), transcript variant beta, |
| | | | mRNA. |
| 5720709 | | | EST7714 human nasopharynx Homo sapiens cDNA, |
| | | | mRNA sequence |
| 160338 | | | UI-E-CL1-aeq-d-09-0-UI.r1 UI-E-CL1 Homo sapiens |
| | | | cDNA clone UI-E-CLI-aeq-d-09-0-UI 5, mRNA |
| | | | sequence |
| 2940093 | | | Homo sapiens clone DTIP1B6 mRNA, CAG repeat |
| | | | region |
| 2490411 | ITGB5 | 3693 | Homo sapiens integrin, beta 5 (ITGB5), mRNA. |
| | | | XM_944688 XM_944693 |
| 5360243 | ITGB5 | 3693 | Homo sapiens integrin, beta 5 (ITGB5), mRNA. |
| | | | XM_944688 XM_944693 |
| 5870274 | FAM129C | 199786 | Homo sapiens family with sequence similarity 129, |
| | | | member C (FAM129C), mRNA. |
| 520086 | FCGR1A | 2209 | Homo sapiens Fc fragment of IgG, high affinity Ia, |
| | | | receptor (CD64) (FCGR1A), mRNA. |
| 2570300 | 1F144 | 10561 | Homo sapiens interferon-induced protein 44 (IF144), |
| | | | mRNA. |
| 2710400 | DSC2 | 1824 | Homo sapiens desmocollin 2 (DSC2), transcript variant |
| | | | Dsc2b, mRNA. |
| 3890592 | | | RST5526 Athersys RAGE Library Homo sapiens |
| | | | cDNA, mRNA sequence |
| 6280543 | OASL | 8638 | Homo sapiens 2′-5′-oligoadenylate synthetase-like |
| | | | (OASL), transcript variant 1, mRNA. |
| 630619 | HPSE | 10855 | Homo sapiens heparanase (HPSE), mRNA. |
| 830681 | C14orf45 | 80127 | Homo sapiens chromosome 14 open reading frame 45 |
| | | | (C14orf45), mRNA. |
| 3420474 | CYP26A1 | 1592 | Homo sapiens cytochrome P450, family 26, subfamily |
| | | | A, polypeptide 1 (CYP26A1), transcript variant 1, |
| | | | mRNA. |
| 6290672 | LOC728417 | 728417 | PREDICTED: Homo sapiens hypothetical protein |
| | | | LOC728417 (LOC728417), mRNA. |
| 2570482 | LOC728417 | 728417 | PREDICTED: Homo sapiens hypothetical protein |
| | | | LOC728417 (LOC728417), mRNA. |
| 6370470 | APOL1 | 8542 | Homo sapiens apolipoprotein L, 1 (APOL1), transcript |
| | | | variant 2, mRNA. |
| 5560471 | FCAR | 2204 | Homo sapiens Fc fragment of IgA, receptor for (FCAR), |
| | | | transcript variant 10, mRNA. |
| 6860164 | CLEC1B | 51266 | Homo sapiens C-type lectin domain family 1, member B |
| | | | (CLEC1B), transcript variant 1, mRNA. |
| 5960682 | RBPMS2 | 348093 | Homo sapiens RNA binding protein with multiple |
| | | | splicing 2 (RBPMS2), mRNA. |
| 6450279 | | | Homo sapiens clone 115D87 T cell receptor beta chain |
| | | | mRNA, partial cds |
| 5270725 | COP1 | 114769 | Homo sapiens caspase-1 dominant-negative inhibitor |
| | | | pseudo-ICE (COP1), transcript variant 2, mRNA. |
| 2030170 | COP1 | 114769 | Homo sapiens caspase-1 dominant-negative inhibitor |
| | | | pseudo-ICE (COP1), transcript variant 2, mRNA. |
| 2450707 | SIGLEC16 | 400709 | Homo sapiens sialic acid binding Ig-like lectin 16 |
| | | | (gene/pseudogene) (SIGLEC16), non-coding RNA. |
| 5570039 | LOC728744 | 728744 | PREDICTED: Homo sapiens hypothetical LOC728744 |
| | | | (LOC728744), mRNA. |
| 3120475 | MAP7 | 9053 | Homo sapiens microtubule-associated protein 7 (MAP7), |
| | | | mRNA. |
| 3400097 | MUC20 | 200958 | Homo sapiens mucin 20, cell surface associated |
| | | | (MUC20), transcript variant S, mRNA. |
| 7040181 | PFKFB2 | 5208 | Homo sapiens 6-phosphofructo-2-kinase/fructose-2,6- |
| | | | biphosphatase 2 (PFKFB2), transcript variant 2, mRNA. |
| 5870138 | VWF | 7450 | Homo sapiens von Willebrand factor (VWF), mRNA. |
| 5900543 | September 2004 | 5414 | Homo sapiens septin 4 (SEPT4), transcript variant 3, |
| | | | mRNA. |
| 3710068 | WARS | 7453 | Homo sapiens tryptophanyl-tRNA synthetase (WARS), |
| | | | transcript variant 2, mRNA. |
| 3180528 | MMP9 | 4318 | Homo sapiens matrix metallopeptidase 9 (gelatinase B, |
| | | | 92 kDa gelatinase, 92 kDa type IV collagenase) (MMP9), |
| | | | mRNA. |
| 2650156 | TAP2 | 6891 | Homo sapiens transporter 2, ATP-binding cassette, sub- |
| | | | family B (MDR/TAP) (TAP2), transcript variant 2, |
| | | | mRNA. |
| 2350445 | GP6 | 51206 | Homo sapiens glycoprotein VI (platelet) (GP6), mRNA. |
| 2350128 | SLIC1 | 124460 | Homo sapiens selectin ligand interactor cytoplasmic-1 |
| | | | (SLIC1), transcript variant 1, mRNA. |
| 5960133 | NFIB | 4781 | Homo sapiens nuclear factor I/B (NFIB), mRNA. |
| 7320270 | PNMA3 | 29944 | Homo sapiens paraneoplastic antigen MA3 (PNMA3), |
| | | | mRNA. |
| 460463 | SMARCD3 | 6604 | Homo sapiens SWI/SNF related, matrix associated, actin |
| | | | dependent regulator of chromatin, subfamily d, member |
| | | | 3 (SMARCD3), transcript variant 1, mRNA. |
| 1780088 | TBXA2R | 6915 | Homo sapiens thromboxane A2 receptor (TBXA2R), |
| | | | transcript variant 2, mRNA. |
| 6330474 | CD151 | 977 | Homo sapiens CD151 molecule (Raph blood group) |
| | | | (CD151), transcript variant 2, mRNA. |
| 1260133 | LOC642334 | 642334 | PREDICTED: Homo sapiens similar to hypothetical |
| | | | protein LOC284701, transcript variant 1 (LOC642334), |
| | | | mRNA. |
| 3420044 | SYN2 | 6854 | Homo sapiens synapsin II (SYN2), transcript variant IIa, |
| | | | mRNA. |
| 4880553 | MFSD9 | 84804 | Homo sapiens major facilitator superfamily domain |
| | | | containing 9 (MFSD9), mRNA. |
| 5310475 | RAD17 | 5884 | Homo sapiens RAD17 homolog (S. pombe) (RAD17), |
| | | | transcript variant 3, mRNA. |
| 2370041 | LRRN3 | 54674 | Homo sapiens leucine rich repeat neuronal 3 (LRRN3), |
| | | | mRNA. |
| 4860753 | ASGR2 | 433 | Homo sapiens asialoglycoprotein receptor 2 (ASGR2), |
| | | | transcript variant 3, mRNA. |
| 3800576 | ASGR2 | 433 | Homo sapiens asialoglycoprotein receptor 2 (ASGR2), |
| | | | transcript variant 3, mRNA. |
| 2600747 | IFIT2 | 3433 | Homo sapiens interferon-induced protein with |
| | | | tetratricopeptide repeats 2 (IFIT2), mRNA. |
| 4280273 | GM2A | 2760 | Homo sapiens GM2 ganglioside activator (GM2A), |
| | | | mRNA. |
| 5820491 | MAP7 | 9053 | Homo sapiens microtubule-associated protein 7 (MAP7), |
| | | | mRNA. |
| 1980524 | GBP4 | 115361 | Homo sapiens guanylate binding protein 4 (GBP4), |
| | | | mRNA. |
| 20075 | C3orf60 | 25915 | Homo sapiens chromosome 3 open reading frame 60 |
| | | | (C3orf60), transcript variant 4, mRNA. |
| 5270674 | GPR68 | 8111 | Homo sapiens G protein-coupled receptor 68 (GPR68), |
| | | | mRNA. |
| 5810689 | PRRG4 | 79056 | Homo sapiens proline rich Gla (G-carboxyglutamic acid) |
| | | | 4 (transmembrane) (PRRG4), mRNA. |
| 4210722 | LOC641693 | 641693 | PREDICTED: Homo sapiens hypothetical protein |
| | | | LOC641693 (LOC641693), mRNA. |
| 2490019 | GNG8 | 94235 | Homo sapiens guanine nucleotide binding protein (G |
| | | | protein), gamma 8 (GNG8), mRNA. |
| 7650440 | BACH2 | 60468 | Homo sapiens BTB and CNC homology 1, basic leucine |
| | | | zipper transcription factor 2 (BACH2), mRNA. |
| 6940427 | ZFP30 | 22835 | Homo sapiens zinc finger protein 30 homolog (mouse) |
| | | | (ZFP30), mRNA. |
| 6110575 | FAIM | 55179 | Homo sapiens Fas apoptotic inhibitory molecule |
| | | | (FAIM), transcript variant 1, mRNA. |
| 4670291 | GAST | 2520 | Homo sapiens gastrin (GAST), mRNA. |
| 3440468 | Gcom1 | 145781 | Homo sapiens GRINL1A combined protein (Gcom1), |
| | | | transcript variant 12, mRNA. |
| 6450685 | PLXNA1 | 5361 | Homo sapiens plexin A1 (PLXNA1), mRNA. |
| 2190538 | C10orf2 | 56652 | Homo sapiens chromosome 10 open reading frame 2 |
| | | | (C10orf2), mRNA. |
| 290437 | HRK | 8739 | Homo sapiens harakiri, BCL2 interacting protein |
| | | | (contains only BH3 domain) (HRK), mRNA. |
| 5890470 | CCR6 | 1235 | Homo sapiens chemokine (C-C motif) receptor 6 |
| | | | (CCR6), transcript variant 2, mRNA. |
| 4920110 | GADD45B | 4616 | Homo sapiens growth arrest and DNA-damage- |
| | | | inducible, beta (GADD45B), mRNA. |
| 1030376 | PLCG1 | 5335 | Homo sapiens phospholipase C, gamma 1 (PLCG1), |
| | | | transcript variant 2, mRNA. |
| 5670100 | TCN2 | 6948 | Homo sapiens transcobalamin II; macrocytic anemia |
| | | | (TCN2), mRNA. |
| 2060086 | | | DB049595 TESTI2 Homo sapiens cDNA clone |
| | | | TESTI2038954 5, mRNA sequence |
| 6420746 | FGF9 | 2254 | Homo sapiens fibroblast growth factor 9 (glia-activating |
| | | | factor) (FGF9), mRNA. |
| 1440341 | C1QC | 714 | Homo sapiens complement component 1, q |
| | | | subcomponent, C chain (C1QC), mRNA. |
| 1190519 | MS4A1 | 931 | Homo sapiens membrane-spanning 4-domains, |
| | | | subfamily A, member 1 (MS4A1), transcript variant 3, |
| | | | mRNA. |
| 2000148 | IFIT1 | 3434 | Homo sapiens interferon-induced protein with |
| | | | tetratricopeptide repeats 1 (IFIT1), transcript variant 2, |
| | | | mRNA. |
| 270240 | SLC26A8 | 116369 | Homo sapiens solute carrier family 26, member 8 |
| | | | (SLC26A8), transcript variant 1, mRNA. |
| 1260445 | SLC26A8 | 116369 | Homo sapiens solute carrier family 26, member 8 |
| | | | (SLC26A8), transcript variant 1, mRNA. |
| 4290037 | NOV | 4856 | Homo sapiens nephroblastoma overexpressed gene |
| | | | (NOV), mRNA. |
| 770400 | LOC653600 | 653600 | PREDICTED: Homo sapiens similar to Neutrophil |
| | | | defensin 1 precursor (HNP-1) (HP-1) (HP1) (Defensin, |
| | | | alpha 1) (LOC653600), mRNA. |
| 2810673 | HOXA9 | 3205 | Homo sapiens homeobox A9 (HOXA9), mRNA. |
| 5960747 | TRIM22 | 10346 | Homo sapiens tripartite motif-containing 22 (TRIM22), |
| | | | mRNA. |
| 3130201 | | | UI-H-BI1-aft-d-04-0-UI.s1 NCI_CGAP_Sub3 Homo |
| | | | sapiens cDNA clone IMAGE: 2722975 3, mRNA |
| | | | sequence |
| 7330292 | FBLN2 | 2199 | Homo sapiens fibulin 2 (FBLN2), transcript variant 2, |
| | | | mRNA. |
| 6550762 | FBLN2 | 2199 | Homo sapiens fibulin 2 (FBLN2), transcript variant 2, |
| | | | mRNA. |
| 4860224 | WARS | 7453 | Homo sapiens tryptophanyl-tRNA synthetase (WARS), |
| | | | transcript variant 1, mRNA. |
| 1940162 | GBP2 | 2634 | Homo sapiens guanylate binding protein 2, interferon- |
| | | | inducible (GBP2), mRNA. |
| 3290072 | | | BX116726 NCI_CGAP_Pr28 Homo sapiens cDNA |
| | | | clone IMAGp998J065569, mRNA sequence |
| 5690609 | ZNF331 | 55422 | Homo sapiens zinc finger protein 331 (ZNF331), |
| | | | transcript variant 1, mRNA. |
| 1770243 | IGF2BP3 | 10643 | Homo sapiens insulin-like growth factor 2 mRNA |
| | | | binding protein 3 (IGF2BP3), mRNA. |
| 4220187 | DYSF | 8291 | Homo sapiens dysferlin, limb girdle muscular dystrophy |
| | | | 2B (autosomal recessive) (DYSF), mRNA. |
| 7330142 | TGFB1I1 | 7041 | Homo sapiens transforming growth factor beta 1 induced |
| | | | transcript 1 (TGFB1I1), transcript variant 2, mRNA. |
| 620750 | CALD1 | 800 | Homo sapiens caldesmon 1 (CALD1), transcript variant |
| | | | 5, mRNA. |
| 3420100 | DSC2 | 1824 | Homo sapiens desmocollin 2 (DSC2), transcript variant |
| | | | Dsc2a, mRNA. |
| 5550397 | APOL6 | 80830 | Homo sapiens apolipoprotein L, 6 (APOL6), mRNA. |
| 2630020 | SUPT3H | 8464 | Homo sapiens suppressor of Ty 3 homolog (S. |
| | | | cerevisiae) (SUPT3H), transcript variant 2, mRNA. |
| 520408 | IFIT3 | 3437 | Homo sapiens interferon-induced protein with |
| | | | tetratricopeptide repeats 3 (IFIT3), mRNA. |
| 4120367 | LPCAT2 | 54947 | Homo sapiens lysophosphatidylcholine acyltransferase 2 |
| | | | (LPCAT2), mRNA. |
| 130463 | FCGBP | 8857 | PREDICTED: Homo sapiens Fc fragment of IgG |
| | | | binding protein (FCGBP), mRNA. |
| 430019 | LOC391045 | 391045 | PREDICTED: Homo sapiens similar to Solute carrier |
| | | | family 2, facilitated glucose transporter member 3 |
| | | | (Glucose transporter type 3, brain) (LOC391045), |
| | | | mRNA. |
| 610519 | TPM1 | 7168 | Homo sapiens tropomyosin 1 (alpha) (TPM1), transcript |
| | | | variant 7, mRNA. |
| 5390168 | SH2D1B | 117157 | Homo sapiens SH2 domain containing 1B (SH2D1B), |
| | | | mRNA. |
| 1940632 | NCAPG2 | 54892 | Homo sapiens non-SMC condensin II complex, subunit |
| | | | G2 (NCAPG2), mRNA. |
| 7100035 | IL28RA | 163702 | Homo sapiens interleukin 28 receptor, alpha (interferon, |
| | | | lambda receptor) (IL28RA), transcript variant 2, mRNA. |
| 6350121 | RASSF1 | 11186 | Homo sapiens Ras association (RalGDS/AF-6) domain |
| | | | family member 1 (RASSF1), transcript variant C, |
| | | | mRNA. |
| 1410221 | S100A12 | 6283 | Homo sapiens S100 calcium binding protein A12 |
| | | | (S100A12), mRNA. |
| 3780092 | TNFRSF21 | 27242 | Homo sapiens tumor necrosis factor receptor |
| | | | superfamily, member 21 (TNFRSF21), mRNA. |
| 1660615 | CCR2 | 1231 | Homo sapiens chemokine (C-C motif) receptor 2 |
| | | | (CCR2), transcript variant A, mRNA. |
| 6270553 | CXCL10 | 3627 | Homo sapiens chemokine (C-X-C motif) ligand 10 |
| | | | (CXCL10), mRNA. |
| 6370414 | CLECL1 | 160365 | Homo sapiens C-type lectin-like 1 (CLECL1), mRNA. |
| 5890095 | LILRA5 | 353514 | Homo sapiens leukocyte immunoglobulin-like receptor, |
| | | | subfamily A (with TM domain), member 5 (LILRA5), |
| | | | transcript variant 1, mRNA. |
| 830563 | PDE9A | 5152 | Homo sapiens phosphodiesterase 9A (PDE9A), |
| | | | transcript variant 2, mRNA. |
| 4120424 | COCH | 1690 | Homo sapiens coagulation factor C homolog, cochlin |
| | | | (Limulus polyphemus) (COCH), mRNA. |
| 2350504 | ECGF1 | 1890 | Homo sapiens endothelial cell growth factor 1 (platelet- |
| | | | derived) (ECGF1), mRNA. |
| 7650128 | ECGF1 | 1890 | Homo sapiens endothelial cell growth factor 1 (platelet- |
| | | | derived) (ECGF1), mRNA. |
| 6040259 | ALOX12 | 239 | Homo sapiens arachidonate 12-lipoxygenase (ALOX12), |
| | | | mRNA. |
| 2360035 | FCRL3 | 115352 | Homo sapiens Fc receptor-like 3 (FCRL3), transcript |
| | | | variant 2, mRNA. |
| 4150369 | GK | 2710 | Homo sapiens glycerol kinase (GK), transcript variant 1, |
| | | | mRNA. |
| 1230746 | ZCCHC6 | 79670 | Homo sapiens zinc finger, CCHC domain containing 6 |
| | | | (ZCCHC6), mRNA. |
| 5390093 | INSL3 | 3640 | Homo sapiens insulin-like 3 (Leydig cell) (INSL3), |
| | | | mRNA. |
| 6450594 | CD79B | 974 | Homo sapiens CD79B antigen (immunoglobulin- |
| | | | associated beta) (CD79B), transcript variant 1, mRNA. |
| 450202 | ELF2 | 1998 | Homo sapiens E74-like factor 2 (ets domain |
| | | | transcription factor) (ELF2), transcript variant 1, mRNA. |
| 2810136 | CAPN11 | 11131 | Homo sapiens calpain 11 (CAPN11), mRNA. |
| 1090497 | CREG1 | 8804 | Homo sapiens cellular repressor of E1A-stimulated |
| | | | genes 1 (CREG1), mRNA. |
| 6420703 | LIN9 | 286826 | Homo sapiens lin-9 homolog (C. elegans) (LIN9), |
| | | | mRNA. |
| 4670458 | September 2004 | 5414 | Homo sapiens septin 4 (SEPT4), transcript variant 2, |
| | | | mRNA. |
| 240594 | ANXA3 | 306 | Homo sapiens annexin A3 (ANXA3), mRNA. |
| 10136 | PML | 5371 | PREDICTED: Homo sapiens promyelocytic leukemia, |
| | | | transcript variant 12 (PML), mRNA. |
| 4890156 | RCAN3 | 11123 | Homo sapiens RCAN family member 3 (RCAN3), |
| | | | mRNA. |
| 4780075 | CEACAM8 | 1088 | Homo sapiens carcinoembryonic antigen-related cell |
| | | | adhesion molecule 8 (CEACAM8), mRNA. |
| 2450670 | HIP1R | 9026 | PREDICTED: Homo sapiens huntingtin interacting |
| | | | protein 1 related (HIP1R), mRNA. |
| 4150600 | EIF1AY | 9086 | Homo sapiens eukaryotic translation initiation factor 1A, |
| | | | Y-linked (EIF1AY), mRNA. |
| 3940504 | CD79A | 973 | Homo sapiens CD79a molecule, immunoglobulin- |
| | | | associated alpha (CD79A), transcript variant 2, mRNA. |
| 1450427 | RTP4 | 64108 | Homo sapiens receptor (chemosensory) transporter |
| | | | protein 4 (RTP4), mRNA. |
| 6350356 | MIPEP | 4285 | Homo sapiens mitochondrial intermediate peptidase |
| | | | (MIPEP), nuclear gene encoding mitochondrial protein, |
| | | | mRNA. |
| 3610706 | BMX | 660 | Homo sapiens BMX non-receptor tyrosine kinase |
| | | | (BMX), mRNA. |
| 2900142 | TDRKH | 11022 | Homo sapiens tudor and KH domain containing |
| | | | (TDRKH), transcript variant 3, mRNA. |
| 6940605 | | | Homo sapiens cDNA clone IMAGE: 5277162 |
| 2350168 | SCN9A | 6335 | Homo sapiens sodium channel, voltage-gated, type IX, |
| | | | alpha subunit (SCN9A), mRNA. |
| 7560593 | OSM | 5008 | Homo sapiens oncostatin M (OSM), mRNA. |
| 4640521 | MAX | 4149 | Homo sapiens MYC associated factor X (MAX), |
| | | | transcript variant 5, mRNA. |
| 2680523 | SAMD4A | 23034 | Homo sapiens sterile alpha motif domain containing 4A |
| | | | (SAMD4A), mRNA. |
| 6980576 | PARP15 | 165631 | Homo sapiens poly (ADP-ribose) polymerase family, |
| | | | member 15 (PARP15), transcript variant 2, mRNA. |
| 6840767 | FRMD3 | 257019 | Homo sapiens FERM domain containing 3 (FRMD3), |
| | | | mRNA. |
| 6100379 | LOC648984 | 648984 | PREDICTED: Homo sapiens similar to Baculoviral IAP |
| | | | repeat-containing protein 1 (Neuronal apoptosis |
| | | | inhibitory protein) (LOC648984), mRNA. |
| 6220671 | PLAUR | 5329 | Homo sapiens plasminogen activator, urokinase receptor |
| | | | (PLAUR), transcript variant 2, mRNA. |
| 4070379 | PCYT2 | 5833 | Homo sapiens phosphate cytidylyltransferase 2, |
| | | | ethanolamine (PCYT2), mRNA. |
| 2100528 | HIST1H3F | 8968 | Homo sapiens histone cluster 1, H3f (HIST1H3F), |
| | | | mRNA. |
| 2350630 | CENPQ | 55166 | Homo sapiens centromere protein Q (CENPQ), mRNA. |
| 2970019 | HIST1H4H | 8365 | Homo sapiens histone cluster 1, H4h (HIST1H4H), |
| | | | mRNA. |
| 7550451 | TOX | 9760 | Homo sapiens thymocyte selection-associated high |
| | | | mobility group box (TOX), mRNA. |
| 1050050 | C9orf45 | 81571 | PREDICTED: Homo sapiens chromosome 9 open |
| | | | reading frame 45 (C9orf45), misc RNA. |
| 3780047 | GBP6 | 163351 | Homo sapiens guanylate binding protein family, member |
| | | | 6 (GBP6), mRNA. |
| 7380400 | GBP6 | 163351 | Homo sapiens guanylate binding protein family, member |
| | | | 6 (GBP6), mRNA. |
| 1740435 | HSPC159 | 29094 | Homo sapiens galectin-related protein (HSPC159), |
| | | | mRNA. |
| 4230102 | SOCS3 | 9021 | Homo sapiens suppressor of cytokine signaling 3 |
| | | | (SOCS3), mRNA. |
| 6110451 | SNX25 | 83891 | Homo sapiens sorting nexin 25 (SNX25), mRNA. |
| 5690463 | C11orf80 | 79703 | Homo sapiens chromosome 11 open reading frame 80 |
| | | | (C11orf80), mRNA. |
| 3060524 | LOC153561 | 153561 | Homo sapiens hypothetical protein LOC153561 |
| | | | (LOC153561), mRNA. |
| 2640671 | LOC642342 | 642342 | PREDICTED: Homo sapiens similar to Contactin- |
| | | | associated protein-like 3 precursor (Cell recognition |
| | | | molecule Caspr3) (LOC642342), mRNA. |
| 2630215 | EXTL2 | 2135 | Homo sapiens exostoses (multiple)-like 2 (EXTL2), |
| | | | transcript variant 1, mRNA. |
| 4040176 | LAMA5 | 3911 | Homo sapiens laminin, alpha 5 (LAMA5), mRNA. |
| 4060008 | CAMK2N1 | 55450 | Homo sapiens calcium/calmodulin-dependent protein |
| | | | kinase II inhibitor 1 (CAMK2N1), mRNA. |
| 7400102 | ASPHD2 | 57168 | Homo sapiens aspartate beta-hydroxylase domain |
| | | | containing 2 (ASPHD2), mRNA. |
| 7160468 | DHRS9 | 10170 | Homo sapiens dehydrogenase/reductase (SDR family) |
| | | | member 9 (DHRS9), transcript variant 1, mRNA. |
| 4490079 | DHRS9 | 10170 | Homo sapiens dehydrogenase/reductase (SDR family) |
| | | | member 9 (DHRS9), transcript variant 1, mRNA. |
| 4290360 | CLEC4F | 165530 | Homo sapiens C-type lectin domain family 4, member F |
| | | | (CLEC4F), mRNA. |
| 5570279 | HIST1H1C | 3006 | Homo sapiens histone cluster 1, H1c (HIST1H1C), |
| | | | mRNA. |
| 3710397 | EFNA1 | 1942 | Homo sapiens ephrin-A1 (EFNA1), transcript variant 1, |
| | | | mRNA. |
| 1500674 | HIST2H3C | 126961 | Homo sapiens histone cluster 2, H3c (HIST2H3C), |
| | | | mRNA. |
| 1300719 | C14orf149 | 112849 | Homo sapiens chromosome 14 open reading frame 149 |
| | | | (C14orf149), mRNA. |
| 2140242 | TNFAIP6 | 7130 | Homo sapiens tumor necrosis factor, alpha-induced |
| | | | protein 6 (TNFAIP6), mRNA. |
| 1030484 | SPINK2 | 6691 | Homo sapiens serine peptidase inhibitor, Kazal type 2 |
| | | | (acrosin-trypsin inhibitor) (SPINK2), mRNA. |

TABLE 12
List of the 320 genes (Treatment Specific 320-Transcript Signature)

| ProbeID | Symbol | Entrez_Gene_ID | ProbeID | Symbol | Entrez_Gene_ID |
|---|---|---|---|---|---|
| 2190349 | KCNJ15 | 3772 | 2230553 | USP32 | 84669 |
| 5860669 | LOC100170939 | 100170939 | 1230376 | ALAS2 | 212 |
| 4060717 | LOC400499 | 400499 | 2030678 | HIST2H2AB | 317772 |
| 5700725 | EPSTI1 | 94240 | 50136 | CMTM5 | 116173 |
| 6100356 | ALPL | 249 | 4290619 | CREB5 | 9586 |
| 2070646 | GPR84 | 53831 | 3360343 | RSAD2 | 91543 |
| 4060626 | ROR1 | 4919 | 5310437 | MYL9 | 10398 |
| 130181 | ANKRD22 | 118932 | 2480327 | MYL9 | 10398 |
| 620136 | LILRA5 | 353514 | 1580750 | LOC100130828 | 100130828 |
| 4860520 | LOC134466 | 134466 | 1010246 | IFI6 | 2537 |
| 5860075 | CAMP | 820 | 1090390 | OAS1 | 4938 |
| 780209 | | | 2140020 | LIMK2 | 3985 |
| 5570398 | FCGR1C | 100132417 | 3390048 | LIMK2 | 3985 |
| 6620209 | FCGR1B | 2210 | 5910632 | SMARCD3 | 6604 |
| 2230575 | LOC642267 | 642267 | 3420008 | SNX6 | 58533 |
| 20048 | HIST2H2BF | 440689 | 7400377 | CEACAM6 | 4680 |
| 3990010 | | | 5050537 | OPLAH | 26873 |
| 1510364 | GBP5 | 115362 | 4900747 | C11orf75 | 56935 |
| 1340241 | C5 | 727 | 1240358 | RAB20 | 55647 |
| 6370768 | ETV7 | 51513 | 7200338 | BMP2K | 55589 |
| 3440095 | PSG3 | 5671 | 1470685 | ITGB3 | 3690 |
| 50445 | LOC728262 | 728262 | 630315 | DHRS9 | 10170 |
| 580347 | LOC388572 | 388572 | 1660021 | RNU4ATAC | 100151683 |
| 4060014 | LOC441124 | 441124 | 6760286 | | |
| 4280632 | GAS6 | 2621 | 3130220 | TMEM158 | 25907 |
| 2490315 | | | 3290292 | LAP3 | 51056 |
| 7100161 | VNN2 | 8875 | 3780187 | GYPB | 2994 |
| 110639 | MAPK14 | 1432 | 1090538 | LOC100128737 | 100128737 |
| 3180220 | GPR109B | 8843 | 4850592 | P2RY14 | 9934 |
| 4860255 | ASPRV1 | 151516 | 4610241 | P2RY14 | 9934 |
| 3390097 | LOC728093 | 728093 | 1340364 | P2RY14 | 9934 |
| 1030463 | PROK2 | 60675 | 6510170 | IFIT3 | 3437 |
| 2970201 | ABHD2 | 11057 | 1500280 | IFIT3 | 3437 |
| 6220735 | MIR720 | 100302198 | 1440360 | GPR141 | 353345 |
| 4200669 | CR1 | 1378 | 7570575 | LOC642788 | 642788 |
| 3940356 | CR1 | 1378 | 4150270 | ANKRD22 | 118932 |
| 7560039 | TAPBP | 6892 | 4230328 | CALD1 | 800 |
| 6620725 | PPAP2C | 8612 | 2140524 | HIST1H3D | 8351 |
| 2030309 | SERPING1 | 710 | 5360626 | SLC26A8 | 116369 |
| 5960731 | SOD2 | 6648 | 770088 | DTX3L | 151636 |
| 3170273 | FER1L3 | 26509 | 1980431 | DOK3 | 79930 |
| 4780044 | LOC389386 | 389386 | 5670465 | ADM | 133 |
| 5130025 | LOC100134703 | 100134703 | 620091 | LOC730234 | 730234 |
| 4120561 | LIMK2 | 3985 | 1820592 | HIST2H2AA3 | 8337 |
| 1510255 | LOC648164 | 648164 | 610451 | HIST2H2AA3 | 8337 |
| 730008 | | | 5220142 | LIMK2 | 3985 |
| 5420646 | CETP | 1071 | 2570041 | WASF3 | 10810 |
| 450424 | CXCR7 | 57007 | 6060053 | LILRA6 | 79168 |
| 3830228 | GPR109A | 338442 | 3870193 | TMEM180 | 79847 |
| 1470382 | IRF7 | 3665 | 4200754 | ST3GAL4 | 6484 |
| 5310445 | KREMEN1 | 83999 | 3710086 | DHRS12 | 79758 |
| 3390301 | KREMEN1 | 83999 | 6980133 | | |
| 5310754 | VNN1 | 8876 | 2490463 | MXI1 | 4601 |
| 7320767 | LOC645822 | 645822 | 3370400 | LOC643313 | 643313 |
| 6840035 | GBP1 | 2633 | 4010564 | TRPM6 | 140803 |
| 2190148 | GBP1 | 2633 | 4900239 | CD274 | 29126 |
| 4260368 | UBE2C | 11065 | 940274 | GK | 2710 |
| 3800398 | FBXO6 | 26270 | 3850630 | HOMER2 | 9455 |
| 5900524 | LACTB | 114294 | 6380672 | CA4 | 762 |
| 6220332 | SLC6A12 | 6539 | 6650242 | IFITM3 | 10410 |
| 510072 | LOC100132317 | 100132317 | 1820750 | STAT1 | 6772 |
| 1400373 | SLA | 6503 | 2970482 | RNU1-5 | 26863 |
| 2360091 | RNU1G2 | 26864 | 1260066 | | |
| 6200577 | CREB5 | 9586 | 6250615 | PGLYRP1 | 8993 |
| 4730059 | BATF2 | 116071 | 5870608 | PPP1R3B | 79660 |
| 460242 | LOC391859 | 391859 | 3870338 | IFI44L | 10964 |
| 2600735 | TLR6 | 10333 | 6660162 | LRG1 | 116844 |
| 3420241 | SLC2A14 | 144195 | 990349 | MCTP2 | 55784 |
| 7050519 | CLEC4D | 338339 | 7510537 | SCO2 | 9997 |
| 7510647 | CACNA1E | 111 | 2510551 | STK3 | 6788 |
| 2450392 | LOC642780 | 642780 | 3190050 | | |
| 4150014 | CEACAM1 | 634 | 1410192 | TDRD9 | 122402 |
| 5700753 | CEACAM1 | 634 | 380082 | LOC646144 | 646144 |
| 1820725 | LRRK2 | 120892 | 1470470 | GPR97 | 222487 |
| 6200193 | TncRNA | 283131 | 5260424 | GPR97 | 222487 |
| 3460053 | SIPA1L2 | 57568 | 4230441 | GPR97 | 222487 |
| 1580435 | TGM2 | 7052 | 2370743 | LOC100132858 | 100132858 |
| 3420593 | LMNB1 | 4001 | 6550259 | CD151 | 977 |
| 4780128 | ATF3 | 467 | 3140487 | BMX | 660 |
| 1690162 | | | 520086 | FCGR1A | 2209 |
| 6590131 | HP | 3240 | 2570300 | IFI44 | 10561 |
| 6330471 | BLK | 640 | 2710400 | DSC2 | 1824 |
| 3440360 | ZNF438 | 220929 | 630619 | HPSE | 10855 |
| 2100458 | LOC729010 | 729010 | 6290672 | LOC728417 | 728417 |
| 6100136 | CASP4 | 837 | 2570482 | LOC728417 | 728417 |
| 6040398 | SLC22A4 | 6583 | 5560471 | FCAR | 2204 |
| 6420750 | TLR2 | 7097 | 3420259 | MIR21 | 406991 |
| 2450364 | VNN3 | 55350 | 2030170 | CARD16 | 114769 |
| 2690477 | LOC648710 | 648710 | 870193 | SP140 | 11262 |
| 630255 | LOC652578 | 652578 | 5570039 | LOC728744 | 728744 |
| 3360431 | ZAK | 51776 | 3360372 | CCRL2 | 9034 |
| 5390328 | KCNJ15 | 3772 | 5390204 | SCARF1 | 8578 |
| 1050215 | KCNJ15 | 3772 | 3990170 | IFI27 | 3429 |
| 6020097 | SERPINA1 | 5265 | 7040181 | PFKFB2 | 5208 |
| 620403 | LOC400759 | 400759 | 5900543 | Sep-04 | 5414 |
| 6280470 | LOC728519 | 728519 | 3180528 | MMP9 | 4318 |
| 5570114 | GADD45G | 10912 | 360377 | TGFA | 7039 |
| 2710091 | FCGR2C | 9103 | 460463 | SMARCD3 | 6604 |
| 6110634 | ERLIN1 | 10613 | 1260133 | LOC642334 | 642334 |
| 7610750 | LOC100134660 | 100134660 | 5870678 | LOC441763 | 441763 |
| 4880537 | LIMA1 | 51474 | 6420102 | GPR109B | 8843 |
| 1050020 | NLRC4 | 58484 | 1980524 | GBP4 | 115361 |
| 3390121 | CASP5 | 838 | 20075 | NDUFAF3 | 25915 |
| 2900524 | CCR2 | 1231 | 5810689 | PRRG4 | 79056 |
| 4760433 | C16orf7 | 9605 | 4210722 | LOC641693 | 641693 |
| 3290458 | FEZ1 | 9638 | 3870170 | LOC728093 | 728093 |
| 2320689 | LOC653610 | 653610 | 7100703 | LOC100132287 | 100132287 |
| 5130767 | MAPK14 | 1432 | 6220739 | GRAMD1B | 57476 |
| 840685 | IL1B | 3553 | 1030376 | PLCG1 | 5335 |
| 1090333 | CDKN2D | 1032 | 4560746 | FCAR | 2204 |
| 1260270 | AIM2 | 9447 | 1440341 | C1QC | 714 |
| 4180148 | LOC650546 | 650546 | 270240 | SLC26A8 | 116369 |
| 5390427 | NAIP | 4671 | 4860746 | LOC642684 | 642684 |
| 5340240 | NAIP | 4671 | 610270 | ELL2 | 22936 |
| 4860600 | MAPK14 | 1432 | 1770243 | IGF2BP3 | 10643 |
| 1010360 | GCH1 | 2643 | 4220187 | DYSF | 8291 |
| 5270377 | LOC728790 | 728790 | 6180427 | G0S2 | 50486 |
| 1510735 | NTN3 | 4917 | 540161 | DNAJC25-GNG10 | 552891 |
| 4010270 | LOC440731 | 440731 | 7650026 | MUC1 | 4582 |
| 1580360 | LOC651612 | 651612 | 5310377 | DSC2 | 1824 |
| 5910019 | C1QB | 713 | 5550397 | APOL6 | 80830 |
| 6620630 | XK | 7504 | 7040592 | ITGAX | 3687 |
| 6580717 | ZMYND15 | 84225 | 520408 | IFIT3 | 3437 |
| 2710709 | FCGR1B | 2210 | 1230671 | LOC100134822 | 100134822 |
| 5670152 | RNU1-3 | 26869 | 1820689 | | |
| 3800050 | ADCY3 | 109 | 1410221 | S100A12 | 6283 |
| 830440 | TLR5 | 7100 | 6270553 | CXCL10 | 3627 |
| 5560270 | CARD17 | 440068 | 5890095 | LILRA5 | 353514 |
| 3930368 | CARD17 | 440068 | 5310451 | BCL6 | 604 |
| 360132 | LHFPL2 | 10184 | 4150369 | GK | 2710 |
| 1780564 | CDC42 | 998 | 5340279 | LOC100133565 | 100133565 |
| 1050025 | CR1 | 1378 | 5390093 | INSL3 | 3640 |
| 4220592 | CACNA2D3 | 55799 | 5960176 | SCARNA22 | 677770 |
| 6290754 | LOC652234 | 652234 | 450202 | ELF2 | 1998 |
| 6420594 | TIFA | 92610 | 4670458 | Sep-04 | 5414 |
| 1070367 | C19orf59 | 199675 | 240594 | ANXA3 | 306 |
| 4260017 | CDK5RAP2 | 55755 | 2030551 | GPR27 | 2850 |
| 3890609 | PLSCR1 | 5359 | 4920228 | FFAR2 | 2867 |
| 3780382 | DKFZp761E198 | 91056 | 4780075 | CEACAM8 | 1088 |
| 3140707 | PARP9 | 83666 | 2320750 | MRVI1 | 10335 |
| 6650064 | SIGLEC5 | 8778 | 7330441 | RNF13 | 11342 |
| 4640270 | SIGLEC5 | 8778 | 1430068 | USF1 | 7391 |
| 1030431 | ACSL1 | 2180 | 3060435 | LOC652750 | 652750 |
| 3360132 | SNX20 | 124460 | 3450521 | ECM1 | 1893 |
| 540025 | IL4R | 3566 | 1450427 | RTP4 | 64108 |
| 2470601 | IL1RN | 3557 | 3610706 | BMX | 660 |
| 60719 | KCNJ2 | 3759 | 3850441 | MAZ | 4150 |
| 5340767 | CEACAM1 | 634 | 360360 | LOC100129343 | 100129343 |
| 2100725 | KIR3DL3 | 115653 | 7560593 | OSM | 5008 |
| 7610440 | XAF1 | 54739 | 3170241 | LOC651524 | 651524 |
| 2710646 | MSRB2 | 22921 | 6100379 | LOC648984 | 648984 |
| 3310341 | FYB | 2533 | 5420678 | LOC100132119 | 100132119 |
| 4640601 | | | 2970019 | HIST1H4H | 8365 |
| 3310376 | TIMM10 | 26519 | 3780047 | GBP6 | 163351 |
| 6280301 | LOC643313 | 643313 | 4230102 | SOCS3 | 9021 |
| 6510707 | FER1L3 | 26509 | 3060524 | LOC153561 | 153561 |
| 3800019 | NCR3 | 259197 | 7400102 | ASPHD2 | 57168 |
| 3830433 | | | 7160468 | DHRS9 | 10170 |
| 1710553 | HSPA6 | 3310 | 2120026 | LOC100134300 | 100134300 |
| 2940685 | C1QA | 712 | 2140242 | TNFAIP6 | 7130 |

TABLE 13A
South Africa 2011 Cohort

| | Active TB Cohort | Latent TB Cohort |
|---|---|---|
| Total | 29 | 38 |
| Gender (total males) | 19 | 17 |
| Ethnicity | 29 Black African | 38 Black African |
| Ave. Age (range, yrs) | 34 (21-65) | 22 (18-44) |
| Previous TB (total no) | 6 | 0 |
| Productive cough (total no.) | 29 | 0 |
| Smear + ve(total no.) | 28 | N/A |
| Night sweats (total no.) | 25 | 0 |
| Weight loss (total no.) | 28 | 0 |
| Chest x-ray perfrm'd (total no) | 1 (1/1 abnormal) | Not done |

Of the 29 untreated active TB patients, 16 were also included in the previous Berry et al study, and of the 38 untreated latent TB patients, 17 were also included in the previous Berry et al study [11]. For study reported in Example 2 herein, all untreated samples were processed again alongside all the other samples.

TABLE 13B
UK 2011 Cohort

| | Treated Active TB Cohort |
|---|---|
| Total | 8 |
| Gender (total males) | 3 |
| Ethnicity | 2 Black African, 3 Indian subcont., |
| | 2 SE Asian, 1 Caucasian |
| Ave. Age (range, yrs) | 343 (19-67) |
| Previous TB (total no.) | 0 |
| Productive cough (total no.) | 6 |
| Smear + ve (total no.) | 4 |
| Night sweats (total no.) | 4 |
| Weight loss (total no.) | 4 |
| Chest x-ray perfrm'd (total no) | 8 (7/8 abnormal) |

### REFERENCES

### References of Example 1

(1) Young D B, Perkins M D, Duncan K, Barry C E, 3rd. Confronting the scientific obstacles to global control of tuberculosis. J Clin Invest 2008; 118:1255-1265.
(2) WHO. Global tuberculosis control. World health organisation. 2010.
(3) Pfyffer G E, Cieslak C, Welscher H M, Kissling P, Rusch-Gerdes S. Rapid detection of mycobacteria in clinical specimens by using the automated bactec 9000 mb system and comparison with radiometric and solid-culture systems. J Clin Microbiol 1997; 35:2229-2234.
(4) CCDC. Center for communicable disease control and prevention. Reported tuberculosis in the United States, 2007. C. U.S. Department of health and human services. Atlanta, ga. 2007.
(5) Boehme C C, Nabeta P, Hillemann D, Nicol M P, Shenai S, Krapp F, Allen J, Tahirli R, Blakemore R, Rustomjee R, Milovic A, Jones M, O'Brien S M, Persing D H, Ruesch-Gerdes S, Gotuzzo E, Rodrigues C, Alland D, Perkins M D. Rapid molecular detection of tuberculosis and rifampin resistance. N Engl J Med 2010; 363:1005-1015.
(6) Mitchison D A. Assessment of new sterilizing drugs for treating pulmonary tuberculosis by culture at 2 months. Am Rev Respir Dis 1993; 147:1062-1063.
(7) Home D J, Royce S E, Gooze L, Narita M, Hopewell P C, Nahid P, Steingart K R. Sputum monitoring during tuberculosis treatment for predicting outcome: Systematic review and meta-analysis. Lancet Infect Dis 2010; 10:387-394.
(8) Walzl G, Ronacher K, Hanekom W, Scriba T J, Zumla A. Immunological biomarkers of tuberculosis. Nat Rev Immunol 2011; 11:343-354.
(9) Berry M P, Graham C M, McNab F W, Xu Z, Bloch S A, Oni T, Wilkinson K A, Banchereau R, Skinner J, Wilkinson R J, Quinn C, Blankenship D, Dhawan R, Cush J J, Mejias A, Ramilo O, Kon O M, Pascual V, Banchereau J, Chaussabel D, O'Garra A. An interferon-inducible neutrophil-driven blood transcriptional signature in human tuberculosis. Nature 2010; 466:973-977.
(10) Benjamini Y, Hochberg Y. Controlling the false discovery rate: A practical and powerful approach to multiple testing. J R Stat Soc Ser A Stat Soc 1995; 57:289-300.
(11) Bland J M, Altman D G. Multiple significance tests: The bonferroni method. BMJ 1995; 310:170.
(12) Haahr M. Random. Org. School of Computer Science and Statistics, Trinity College, Dublin 1998.
(13) Quackenbush J. Computational analysis of microarray data. Nat Rev Genet 2001; 2:418-427.
(14) Pankla R, Buddhisa S, Berry M, Blankenship D M, Bancroft G J, Banchereau J, Lertmemongkolchai G, Chaussabel D. Genomic transcriptional profiling identifies a candidate blood biomarker signature for the diagnosis of septicemic melioidosis. Genome Biol 2009; 10:R127.
(15) Mkhize-Kwitshana Z L, Taylor M, Jooste P, Mabaso M L, Walzl G. The influence of different helminth infection phenotypes on immune responses against hiv in co-infected adults in south africa. BMC Infect Dis 2011; 11:273.
(16) Luo C, Chen M, Xu H. Complement gene expression and regulation in mouse retina and retinal pigment epithelium/choroid. Mol Vis 2011; 17:1588-1597.
(17) Walker D G, Kim S U, McGeer P L. Complement and cytokine gene expression in cultured microglial derived from postmortem human brains. J Neurosci Res 1995; 40:478-493.
(18) Khoo J J, Forster S, Mansell A. Toll-like receptors as interferon-regulated genes and their role in disease. J Interferon Cytokine Res 2011; 31:13-25.
(19) Moran L B, Duke D C, Graeber M B. The microglial gene regulatory network activated by interferon-gamma. J Neuroimmunol 2007; 183:1-6.
(20) Bennett L, Palucka A K, Arce E, Cantrell V, Borvak J, Banchereau J, Pascual V. Interferon and granulopoiesis signatures in systemic lupus erythematosus blood. J Exp Med 2003; 197:711-723.
(21) Maertzdorf J, Repsilber D, Parida S K, Stanley K, Roberts T, Black G, Walzl G, Kaufmann S H. Human gene expression profiles of susceptibility and resistance in tuberculosis. Genes Immun 2011; 12:15-22.
(22) Maertzdorf J, Ota M, Repsilber D, Mollenkopf H J, Weiner J, Hill P C, Kaufmann S H. Functional correlations of pathogenesis-driven gene expression signatures in tuberculosis. PLoS One 2011; 6:e26938.
(23) Nahid P, Saukkonen J, Mac Kenzie W, Johnson J L, Phillips P J, Andersen J, Bliven E, Belisle J, Boom H, Luetkemeyer A, Campbell T, Eisenach K, Hafner R, Lennox J, Makhene M, Swindells S, Villarino E, Weiner M, Benson C, Burman W. Tuberculosis biomarker and surrogate endpoint research roadmap. Am J Respir Crit Care Med 2011; 184:972-979.
(24) Perrin F M, Lipman M C, McHugh T D, Gillespie S H. Biomarkers of treatment response in clinical trials of novel antituberculosis agents. Lancet Infect Dis 2007; 7:481-490.
(25) Wallis R S, Pai M, Menzies D, Doherty T M, Walzl G, Perkins M D, Zumla A. Biomarkers and diagnostics for tuberculosis: Progress, needs, and translation into practice. Lancet 2010; 375:1920-1937.
(26) Walzl G, Ronacher K, Djoba Siawaya J F, Dockrell H M. Biomarkers for tb treatment response: Challenges and future strategies. J Infect 2008; 57:103-109.
(27) Koth L L, Solberg O D, Peng J C, Bhakta N R, Nguyen C P, Woodruff P G. Sarcoidosis blood transcriptome reflects lung inflammation and overlaps with tuberculosis. Am J Respir Crit Care Med 2011; 184:1153-1163.
(28) Mistry R, Cliff J M, Clayton C L, Beyers N, Mohamed Y S, Wilson P A, Dockrell H M, Wallace D M, van Helden P D, Duncan K, Lukey P T. Gene-expression patterns in whole blood identify subjects at risk for recurrent tuberculosis. J Infect Dis 2007; 195:357-365.
(29) Joosten S A, Goeman J J, Sutherland J S, Opmeer L, de Boer K G, Jacobsen M, Kaufmann S H, Finos L, Magis-Escurra C, Ota M O, Ottenhoff T H, Haks M C. Identification of biomarkers for tuberculosis disease using a novel dual-color rt-mlpa assay. Genes Immun 2012; 13:71-82.

### References of Example 2

[1] Young D B, Perkins M D, Duncan K, Barry C E 3rd (2008) Confronting the scientific obstacles to global control of tuberculosis. J Clin Invest 118: 1255-1265.
[2] WHO (2010) Global tuberculosis control. World Health Organisation.
[3] Pfyffer G E, Cieslak C, Welscher H M, Kissling P, Rusch-Gerdes S (1997) Rapid detection of mycobacteria in clinical specimens by using the automated BACTEC 9000MB system and comparison with radiometric and solid-culture systems. J Clin Microbiol 35: 2229-2234.
[4] Boehme C C, Nabeta P, Hillemann D, Nicol M P, Shenai S, et al. (2010) Rapid molecular detection of tuberculosis and rifampin resistance. N Engl J Med 363: 1005-1015.
[5] CCDC (2007) Center for Communicable Disease Control and Prevention. Reported Tuberculosis in the United States, 2007. C. U.S. Department of Health and Human Services. Atlanta, Ga.
[6] Mitchison D A (1993) Assessment of new sterilizing drugs for treating pulmonary tuberculosis by culture at 2 months. Am Rev Respir Dis 147: 1062-1063.
[7] Home D J, Royce S E, Gooze L, Narita M, Hopewell P C, et al. (2010) Sputum monitoring during tuberculosis treatment for predicting outcome: systematic review and meta-analysis. Lancet Infect Dis 10: 387-394.
[8] Walzl G, Ronacher K, Hanekom W, Scriba T J, Zumla A (2011) Immunological biomarkers of tuberculosis. Nat Rev Immunol 11: 343-354.
[9] Reis-Filho J S, Pusztai L (2011) Gene expression profiling in breast cancer: classification, prognostication, and prediction. Lancet 378: 1812-1823.
[10] Pascual V, Chaussabel D, Banchereau J (2010) A genomic approach to human autoimmune diseases. Annu Rev Immunol 28: 535-571.
[11] Berry M P, Graham C M, McNab F W, Xu Z, Bloch S A, et al. (2010) An interferon-inducible neutrophil-driven blood transcriptional signature in human tuberculosis. Nature 466: 973-977.
[12] Haahr M (1998) random.org. School of Computer Science and Statistics, Trinity College, Dublin.
[13] Benjamini Y, Hochberg Y (1995) Controlling the false discovery rate: a practical and powerful approach to multiple testing. J R Stat Soc Ser A Stat Soc 57: 289-300.
[14] Bland J M, Altman D G (1995) Multiple significance tests: the Bonferroni method. BMJ 310: 170.
[15] Quackenbush J (2001) Computational analysis of microarray data. Nat Rev Genet 2: 418-427.
[16] Pankla R, Buddhisa S, Berry M, Blankenship D M, Bancroft G J, et al. (2009) Genomic transcriptional profiling identifies a candidate blood biomarker signature for the diagnosis of septicemic melioidosis. Genome Biol 10: R127.
[17] Nahid P, Saukkonen J, Mac Kenzie W, Johnson J L, Phillips P J, et al. (2011) Tuberculosis Biomarker and Surrogate Endpoint Research Roadmap. Am J Respir Crit Care Med 184: 972-979.
[18] Perrin F M, Lipman M C, McHugh T D, Gillespie S H (2007) Biomarkers of treatment response in clinical trials of novel antituberculosis agents. Lancet Infect Dis 7: 481-490.
[19] Wallis R S, Pai M, Menzies D, Doherty T M, Walzl G, et al. (2010) Biomarkers and diagnostics for tuberculosis: progress, needs, and translation into practice. Lancet 375: 1920-1937.
[20] Walzl G, Ronacher K, Djoba Siawaya J F, Dockrell H M (2008) Biomarkers for T B treatment response: challenges and future strategies. J Infect 57: 103-109.
[21] Gumbo T, Louie A, Liu W, Brown D, Ambrose P G, et al. (2007) Isoniazid bactericidal activity and resistance emergence: integrating pharmacodynamics and pharmacogenomics to predict efficacy in different ethnic populations. Antimicrob Agents Chemother 51: 2329-2336.
[22] Jindani A, Aber V R, Edwards E A, Mitchison D A (1980) The early bactericidal activity of drugs in patients with pulmonary tuberculosis. Am Rev Respir Dis 121: 939-949.
[23] de Steenwinkel J E, de Knegt G J, ten Kate M T, van Belkum A, Verbrugh H A, et al. (2010) Time-kill kinetics of anti-tuberculosis drugs, and emergence of resistance, in relation to metabolic activity of Mycobacterium tuberculosis. J Antimicrob Chemother 65: 2582-2589.
[24] Koth L L, Solberg O D, Peng J C, Bhakta N R, Nguyen C P, et al. (2011) Sarcoidosis Blood Transcriptome Reflects Lung Inflammation and Overlaps with Tuberculosis. Am J Respir Crit Care Med 184: 1153-1163.
[25] Maertzdorf J, Repsilber D, Parida S K, Stanley K, Roberts T, et al. (2011) Human gene expression profiles of susceptibility and resistance in tuberculosis. Genes Immun 12: 15-22.
[26] Maertzdorf J, Ota M, Repsilber D, Mollenkopf H J, Weiner J, et al. (2011) Functional correlations of pathogenesis-driven gene expression signatures in tuberculosis. PLoS One 6: e26938.
[27] Mistry R, Cliff J M, Clayton C L, Beyers N, Mohamed Y S, et al. (2007) Gene-expression patterns in whole blood identify subjects at risk for recurrent tuberculosis. J Infect Dis 195: 357-365.
[28] Joosten S A, Goeman J J, Sutherland J S, Opmeer L, de Boer K G, et al. (2012) Identification of biomarkers for tuberculosis disease using a novel dual-color RT-MLPA assay. Genes Immun 13: 71-82.

What is claimed is:

1. A method for treating tuberculosis in a patient suspected of having active tuberculosis comprising:
measuring expression levels of genes in a biological sample from the patient to generate a first gene expression profile;
administering a first anti-mycobacterial treatment to the patient;
measuring expression levels of the same genes measured in the first gene expression profile in a second biological sample from the patient to generate a second gene expression profile, wherein the second biological sample is obtained at two months or less after administration of the first anti-mycobacterial treatment; and
calculating a temporal molecular response percentage (% TMR), wherein:
% TMR=(x/y)*100; wherein x is equal to the number of measured genes whose expression levels are greater than two-fold different between the first and second gene expression profile measurements and y is equal to the total number of measured genes in the first gene expression profile;
continuing treatment with the same anti-mycobacterial drug(s) administered in the first anti-mycobacterial treatment when the % TMR is more than 10%; and
administering a second anti-mycobacterial treatment to the patient comprising different anti-mycobacterial drugs when the % TMR is less than 10%.

2. (canceled)

3. The method of claim 1, wherein the biological sample is blood.

4-5. (canceled)

6. The method of claim 1, wherein genes of the gene expression profile comprise genes selected from Table 1, 3, 4, 5, 6, 7, 8, 9, 10, 11, or 12.

7. The method of claim 1, wherein genes of the gene expression profile comprise 1, 2, 3, 4, 5, 6 or more genes related to IFN Signaling selected from the group consisting of IF135, IFIT1, IFIT3, IFITM1, IRF1, JAK2, SOCS1, STAT1, and TAP1.

8. The method of claim 1, wherein genes of the gene expression profile comprise 1, 2, 3, 4, 5, 6 or more genes related to T and B cell signaling selected from the group consisting of CD40LG, CD79A, CD79B, FAS, FCER1G, IL15, IL1B, IL1RN, SLAMF1, TLR2, TLR5, and TNFSF13B.

9. The method of claim 1, wherein genes of the gene expression profile comprise 1, 2, 3, 4, 5, 6 or more genes related to a complement system selected from the group consisting of C2, C1QB, C1QC, C4BPA, CD59, CR1, and SERPING1.

10. The method of claim 1, wherein genes of the gene expression profile comprise 1, 2, 3, 4, 5, 6 or more genes having a role in pattern recognition selected from the group consisting of C5, C1QB, C1QC, CASH, IFIH1, IL1B, IRF7, NLRC4, NOD2, TLR2, and TLR5.

11. The method of claim 1, wherein genes of the gene expression profile comprise one or more genes related to JAK family kinases in IL-6 type cytokine signaling selected from the group consisting of MAPK14, OSM, SOCS1, SOCS3, and STAT1.

12. The method of claim 1, wherein genes of the gene expression profile comprise 1, 2, 3, 4, 5, 6 or more genes having a role in communication between innate and adaptive immune cells selected from the group consisting of CD86, CD40LG, CXCL10, FCER1G, IL15, IL1B, IL1RN, TLR2, TLR5, TLR8, TNFRSF13B, and TNFSF13B.

13. The method of claim 1, wherein genes of the gene expression profile comprise 1, 2, 3, 4, 5, 6 or more genes related to dendritic cell maturation selected from the group consisting of CD86, CD40LG, CREB5, FCER1G, FCGR1A, FCGR1B, IL15, IL1B, IL1RN, IL23A, JAK2, MAPK14, STAT1, STAT2, and TLR2.

14. The method of claim 1, wherein genes of the gene expression profile comprise 1, 2, 3, 4, 5, 6 or more genes related to TREM signaling selected from the group consisting of CASP1, CASP5, IL1B, ITGAX, JAK2, NOD2, PLCG1, TLR2, and TLR5.

15. The method of claim 1, wherein genes of the gene expression profile comprise 1, 2, 3, 4, 5, 6 or more genes related to a role of macrophages, fibroblasts, or endothelial cells in rheumatoid arthritis selected from the group consisting of C5, CREB5, F2RL1, FCGR1A, IL15, IL18R1, IL18RAP, IL1B, IL1RN, IRAK3, JAK2, MAPK14, NFAT5, OSM, PDGFA, PLCG1, SOCS1, SOCS3, TLR2, TLR5, TNFSF13B, and TRAF5.

16. The method of claim 1, wherein genes of the gene expression profile comprise 1, 2, 3, 4, 5, 6 or more genes related to systemic lupus erythematous signaling selected from the group consisting of C5, CD3E, CD40LG, CD79A, CD79B, FCER1G, FCGR1A, FCGR1B, FCGR1C, FCGR2C, FCGR3B, IL1B, IL1RN, LCK, NFAT5, PLCG1, and TNFSF13B.

17. The method of claim 1, wherein genes of the gene expression profile comprise 1, 2, 3, 4, 5, 6 or more different genes selected from the group consisting of IF135, IFIT1, IFIT3, IFITM1, IRF1, JAK2, SOCS1, STAT1, TAP1, CD40LG, CD79A, CD79B, FAS, FCER1G, IL15, IL1B, IL1RN, SLAMF1, TLR2, TLR5, TNFSF13B, C2, C1QB, C1QC, C4BPA, CD59, CR1, SERPING1, C5, CASP1, IFIH1, IL1B, IRF7, NLRC4, NOD2, MAPK14, OSM, SOCS3, CD86, CXCL10, FCER1G, TLR8, CD86, CREB5, FCGR1A, FCGR1B, IL15, IL23A, STAT2, CASP5, ITGAX, PLCG1, F2RL1, IL18R1, IL18RAP, IRAK3, NFAT5, PDGFA, PLCG1, TRAF5, CD3E, FCGR1C, FCGR2C, FCGR3B, and LCK.

18. The method of claim 1, wherein the second time point is 14, 13, 12, 11, 10, 9, 8, 7, 6, 5, 4, 3, or 2 days or less, or 1 day or less, after commencement.

19-24. (canceled)

25. The method of claim 1, wherein the first or second anti-mycobacterial treatment comprises treatment with rifampin, pyrazinamide, isoniazid, ethambutol, rifampicin, levofloxacin, moxifloxacin, prothioniamide, ethionamide, cycloserine, amikacin, streptomycin, kanamycin, para-amino salicylic acid, capreomycin, linezolid, TMC-205, or derivatives, or combinations thereof.

26-51. (canceled)

52. The method of claim 1, wherein the second time point is between 2 weeks and less than 2 months after the commencement of treatment.

53-60. (canceled)

61. The method of claim 1, wherein treatment with the same anti-mycobacterial drug(s) administered in the first anti-mycobacterial treatment is continued when the % TMR is at least 19 percent and a second anti-mycobacterial treatment is administered to the patient comprising different anti-mycobacterial drugs when the % TMR is less than 19%.

62-63. (canceled)

64. The method of claim 1, treatment with the same anti-mycobacterial drug(s) administered in the first anti-mycobacterial treatment is continued when the % TMR is at least 40 percent and a second anti-mycobacterial treatment is administered to the patient comprising different anti-mycobacterial drugs when the % TMR is less than 40%.

65-80. (canceled)

81. The method of claim 1, wherein the first anti-mycobacterial treatment comprises one or more of isoniazid, rifampin, rifapentine, ethambutol, and pyrazinamide.

82. The method of claim 1, wherein the second anti-mycobacterial treatment comprises one or more of levofloxacin, moxifloxacin, prothioniamide, ethionamide, cycloserine, amikacin, streptomycin, kanamycin, para-amino salicylic acid, capreomycin, linezolid, and TMC-205.
